(19) United States
(12) Patent Application Publication
(10) Pub. No.: US 2024/0117353 A1
(43) Pub. Date: Apr. 11, 2024

(54) COMPOSITIONS FOR TREATMENT OF CONDITIONS AND DISEASES ASSOCIATED WITH POLYCYSTIN EXPRESSION

(71) Applicant: Stoke Therapeutics, Inc., Bedford, MA (US)

(72) Inventors: Isabel Aznarez, Jamaica Plain, MA (US); Jacob Albert Kach, Jamaica Plain, MA (US)

(21) Appl. No.: 18/364,244

(22) Filed: Aug. 2, 2023

Related U.S. Application Data
(63) Continuation of application No. PCT/US2022/015074, filed on Feb. 3, 2022.
(60) Provisional application No. 63/145,288, filed on Feb. 3, 2021.

(51) Int. Cl. C12N 15/113; C07K 14/47; C12N 15/86

(52) U.S. Cl. CPC C12N 15/113; C07K 14/47; C12N 15/86; C12N 2310/313; C12N 2310/314; C12N 2310/321; C12N 2310/322

(57) ABSTRACT

Alternative splicing events in genes can lead to non-productive mRNA transcripts which in turn can lead to aberrant protein expression, and therapeutic agents which can target the alternative splicing events in genes can modulate the expression level of functional proteins in patients and/or inhibit aberrant protein expression. Such therapeutic agents can be used to treat a condition or disease caused by protein deficiency.

### CROSS-REFERENCE

This application is a continuation of international patent application no. PCT/US2022/015074, filed Feb. 3, 2022, which claims the benefit of U.S. Provisional Application No. 63/145,288, filed Feb. 3, 2021, each of which is incorporated herein by reference in its entirety.

### SEQUENCE LISTING

The instant application contains a Sequence Listing which has been submitted electronically in XML format and is hereby incorporated by reference in its entirety. Said XML copy, created on Nov. 25, 2023, is named 47991_732_301_SL.xml and is 552,720 bytes in size.

### BACKGROUND

Alternative splicing events in genes can lead to non-productive mRNA transcripts which in turn can lead to aberrant protein expression, and therapeutic agents which can target the alternative splicing events in genes can modulate the expression level of functional proteins in patients and/or inhibit aberrant protein expression. Such therapeutic agents can be used to treat a condition or disease caused by protein deficiency.

### SUMMARY

Described herein, in certain embodiments, is a method of modulating expression of a target protein in a cell having a pre-mRNA that is transcribed from a target gene and that comprises a non-sense mediated RNA decay-inducing exon (NMD exon), the method comprising: contacting an agent or a vector encoding the agent to the cell, whereby the agent modulates splicing of the NMD exon from the pre-mRNA, thereby modulating the level of a processed mRNA that is processed from the pre-mRNA, and modulating the expression of the target protein in the cell, wherein the target protein is encoded by a PKD2 gene.

Described herein, in certain embodiments, is a method of treating or reducing the likelihood of developing a disease or condition in a subject in need thereof by modulating expression of a target protein in a cell of the subject, comprising: contacting an agent or a vector encoding the agent to the cell of the subject, whereby the agent modulates splicing of a non-sense mediated mRNA decay-inducing exon (NMD exon) from a pre-mRNA that is transcribed from a target gene and that comprises the NMD exon, thereby modulating the level of a processed mRNA that is processed from the pre-mRNA, and modulating the expression of the target protein in the cell of the subject, wherein the target protein encoded by a PKD2 gene.

In some embodiments, the target protein is polycystin 2.

In some embodiments, the disease or condition is a disease or condition associated with a deficiency in amount or activity of polycystin 2. In some embodiments, the disease or condition is a disease or condition associated with a deficiency in amount or activity of polycystin 1.

In some embodiments, the disease or condition is a disease or condition associated with a deficiency in amount or activity of a protein that polycystin 2 functionally augments, compensates for, replaces, or functionally interacts with.

In some embodiments, the agent: (a) binds to a targeted portion of the pre-mRNA; (b) modulates binding of a factor involved in splicing of the NMD exon; or (c) a combination of (a) and (b).

In some embodiments, the agent interferes with binding of the factor involved in splicing of the NMD exon to a region of the targeted portion.

In some embodiments, the targeted portion of the pre-mRNA is proximal to the NMD exon.

In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of the 5′ end of the NMD exon.

In some embodiments, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of the 5′ end of the NMD exon.

In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of the 3′ end of the NMD exon.

In some embodiments, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of the 3′ end of the NMD exon.

In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site of GRCh38/hg38: chr4:88031085.

In some embodiments, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of genomic site of GRCh38/hg38: chr4:88031085.

In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site of GRCh38/hg38: chr4:88031140.

In some embodiments, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of genomic site of GRCh38/hg38: chr4:88031140.

In some embodiments, the targeted portion of the pre-mRNA is located in an intronic region between two canonical exonic regions of the pre-mRNA, and wherein the intronic region contains the NMD exon.

In some embodiments, the targeted portion of the pre-mRNA at least partially overlaps with the NMD exon.

In some embodiments, the targeted portion of the pre-mRNA at least partially overlaps with an intron upstream or downstream of the NMD exon.

In some embodiments, the targeted portion of the pre-mRNA comprises 5′ NMD exon-intron junction or 3′ NMD exon-intron junction.

In some embodiments, the targeted portion of the pre-mRNA is within the NMD exon.

In some embodiments, the targeted portion of the pre-mRNA comprises about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon.

In some embodiments, the NMD exon comprises a sequence with at least 80%, at least 90%, or 100% sequence identity to a sequence selected from the group consisting of the sequences listed in Table 2.

In some embodiments, the NMD exon comprises a sequence selected from the group consisting of the sequences listed in Table 2.

In some embodiments, the pre-mRNA comprises a sequence with at least about 80%, 85%, 90%, 95%, 97%, or 100% sequence identity to a sequence selected from the group consisting of the sequences listed in Table 2 or Table 3.

In some embodiments, the pre-mRNA is encoded by a genetic sequence with at least about 80%, 85%, 90%, 95%, 97%, or 100% sequence identity to a sequence selected from the group consisting of the sequences listed in Table 2 or Table 3.

In some embodiments, the targeted portion of the pre-mRNA comprises a sequence with at least 80%, 85%, 90%, 95%, 97%, or 100% sequence identity to a region comprising at least 8 contiguous nucleic acids of a sequence selected from the group consisting of the sequences listed in Table 2 or Table 3.

In some embodiments, the agent is an antisense oligomer (ASO) and wherein the ASO comprises a sequence that is at least about 80%, 85%, 90%, 95%, 97%, or 100% sequence identity to at least 8, 9, 10, 11, 12, 13, 14, 15, 16, 17 or 18 contiguous nucleic acids of a sequence selected from the group consisting of the sequences listed in Table 4.

In some embodiments, the targeted portion of the pre-mRNA is within the non-sense mediated RNA decay-inducing exon GRCh38/hg38: chr4:88031085-88031140.

In some embodiments, the targeted portion of the pre-mRNA is upstream or downstream of the non-sense mediated RNA decay-inducing exon GRCh38/hg38: chr4:88031085-88031140.

In some embodiments, the targeted portion of the pre-mRNA comprises an exon-intron junction of the non-sense mediated RNA decay-inducing exon GRCh38/hg38: chr4:88031085-88031140.

In some embodiments, the polycystin 2 expressed from the processed mRNA is full-length polycystin 2 or wild-type polycystin 2.

In some embodiments, the polycystin 2 expressed from the processed mRNA is at least partially functional as compared to wild-type polycystin 2.

In some embodiments, the polycystin 2 expressed from the processed mRNA is at least partially functional as compared to full-length wild-type polycystin 2.

In some embodiments, the agent promotes exclusion of the NMD exon from the pre-mRNA, thereby modulating the level of a processed mRNA that is processed from the pre-mRNA and that lacks the NMD exon.

In some embodiments, the exclusion of the NMD exon from the pre-mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to exclusion of the NMD exon from the pre-mRNA in a control cell.

In some embodiments, the method results in an increase in the level of the processed mRNA in the cell.

In some embodiments, the level of the processed mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to a level of the processed mRNA in a control cell.

In some embodiments, the agent increases the expression of the target protein in the cell.

In some embodiments, a level of the target protein is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to a level of the target protein produced in a control cell.

In some embodiments, a processed mRNA containing the NMD exon comprises a premature termination codon (PTC). In some embodiments, the premature termination codon (PTC) is downstream of the NMD exon. In some embodiments, the NMD exon comprises a premature termination codon (PTC).

In some embodiments, the disease or condition is associated with a loss-of-function mutation in the target gene or the target protein.

In some embodiments, the disease or condition is associated with haploinsufficiency of the target gene, and wherein the subject has a first allele encoding functional polycystin 2, and a second allele from which polycystin 2 is not produced or produced at a reduced level, or a second allele encoding nonfunctional polycystin 2 or partially functional polycystin 2.

In some embodiments, one or both alleles are hypomorphs or partially functional.

In some embodiments, the disease or condition is selected from the group consisting of: polycystic kidney disease with or without polycystic liver disease, autosomal dominant polycystic kidney disease, and intracranial aneurysm.

In some embodiments, the disease or condition is associated with a mutation of a PKD1 or PKD2 gene, wherein the subject has a first allele encoding from which: (i) the target protein is not produced or produced at a reduced level compared to a wild-type allele; or (ii) the target protein produced is nonfunctional or partially functional compared to a wild-type allele, and a second allele from which: (iii) the target protein is produced at a reduced level compared to a wild-type allele and the target protein produced is at least partially functional compared to a wild-type allele; or (iv) the target protein produced is partially functional compared to a wild-type allele.

In some embodiments, the disease or condition is selected from the group consisting of: polycystic kidney disease with or without polycystic liver disease, autosomal dominant polycystic kidney disease, and intracranial aneurysm.

In some embodiments, the mutation is a hypomorphic mutation.

In some embodiments, the agent promotes exclusion of the NMD exon from the pre-mRNA, thereby modulating the level of a processed mRNA that is processed from the pre-mRNA and that lacks the NMD exon and increases the expression of the target protein in the cell.

In some embodiments, the agent is an antisense oligomer (ASO) and wherein the antisense oligomer comprises a backbone modification comprising a phosphorothioate linkage or a phosphorodiamidate linkage.

In some embodiments, the agent is an antisense oligomer (ASO) and wherein the antisense oligomer comprises a phosphorodiamidate morpholino, a locked nucleic acid, a peptide nucleic acid, a 2′-O-methyl, a 2′-Fluoro, or a 2′-O-methoxyethyl moiety.

In some embodiments, the agent is an antisense oligomer (ASO) and wherein the antisense oligomer comprises at least one modified sugar moiety.

In some embodiments, each sugar moiety is a modified sugar moiety.

In some embodiments, the agent is an antisense oligomer (ASO) and wherein the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

In some embodiments, the agent is an antisense oligomer (ASO) and wherein the antisense oligomer is at least 80%, at least 85%, at least 90%, at least 95%, at least 98%, at least 99%, or 100%, complementary to the targeted portion of the pre-mRNA.

In some embodiments, the method comprises contacting a vector encoding the agent to the cell.

In some embodiments, the agent is a polynucleotide comprising an antisense oligomer.

In some embodiments, the vector is a viral vector.

In some embodiments, the viral vector is an adenovirus-associated viral vector.

In some embodiments, the polynucleotide further comprises a modified snRNA.

In some embodiments, the modified human snRNA is a modified U1 snRNA or a modified U7 snRNA.

In some embodiments, the modified human snRNA is a modified U7 snRNA and wherein the antisense oligomer has a sequence at least 75%, at least 80%, at least 85%, at least 90%, at least 95%, at least 98%, at least 99%, or 100% identical to a sequence listed in Table 4 or Table 5.

In some embodiments, the method further comprises assessing processed mRNA level or expression level of the target protein.

In some embodiments, the subject is a human.

In some embodiments, the subject is a non-human animal.

In some embodiments, the subject is a fetus, an embryo, or a child.

In some embodiments, the cells are ex vivo.

In some embodiments, the agent is administered by intrathecal injection, intracerebroventricular injection, intraperitoneal injection, intramuscular injection, subcutaneous injection, intravitreal, or intravenous injection of the subject.

In some embodiments, the method further comprises administering a second therapeutic agent to the subject.

In some embodiments, the second therapeutic agent is a small molecule.

In some embodiments, the second therapeutic agent is an antisense oligomer.

In some embodiments, the second therapeutic agent corrects intron retention.

In some embodiments, the method treats the disease or condition.

Described herein, in certain embodiments, is a composition comprising an agent or a vector encoding the agent that modulates splicing of a non-sense mediated RNA decay-inducing exon (NMD exon) from a pre-mRNA that is transcribed from a target gene and that comprises the NMD exon, thereby modulating the level of a processed mRNA that is processed from the pre-mRNA, and modulating expression of a target protein in a cell having the pre-mRNA, wherein the target protein is encoded by a PKD2 gene.

Described herein, in certain embodiments, is a composition comprising an agent or a vector encoding the agent that modulates splicing of a non-sense mediated mRNA decay-inducing exon (NMD exon) from a pre-mRNA that is transcribed from a target gene and that comprises the NMD exon, thereby treating a disease or condition in a subject in need thereof by modulating the level of a processed mRNA that is processed from the pre-mRNA, and modulating expression of a target protein in a cell of the subject, wherein the target protein is encoded by a PKD2 gene.

Described herein, in certain embodiments, is a pharmaceutical composition comprising the composition as described herein; and a pharmaceutically acceptable excipient and/or a delivery vehicle.

Described herein, in certain embodiments, is a composition comprising a non-sense mediated RNA decay alternative splice site (NSASS) modulating agent or a viral vector encoding the agent, wherein the agent modulates expression of a target protein in a cell comprising a pre-mRNA that is transcribed from a target gene and encodes the target protein, wherein the pre-mRNA comprises an alternative 5′ splice-site downstream of a canonical 5′ splice-site, wherein a processed mRNA that is produced by splicing of the pre-mRNA at the alternative 5′ splice-site undergoes non-sense mediated RNA decay, wherein the agent modulates processing of the pre-mRNA by modulating splicing at the alternative 5′ splice-sites; and wherein the target gene is PKD2.

In some embodiments, the agent modulates processing of the pre-RNA by preventing or decreasing splicing at the alternative 5′ splice-site.

In some embodiments, the agent modulates processing of the pre-RNA by promoting or increasing splicing at the canonical 5′ splice-site.

In some embodiments, modulating the splicing of the pre-mRNA at the alternative 5′ splice-site increases the expression of the target protein in the cell.

In some embodiments, the processed mRNA that is produced by splicing of the pre-mRNA at the alternative 5′ splice-site comprises a premature termination codon (PTC).

In some embodiments, the agent is a small molecule.

In some embodiments, the agent is a polypeptide.

In some embodiments, the polypeptide is a nucleic acid binding protein.

In some embodiments, the nucleic acid binding protein contains a TAL-effector or zinc finger binding domain.

In some embodiments, the nucleic acid binding protein is a Cas family protein.

In some embodiments, the polypeptide is accompanied by or complexed with one or more nucleic acid molecules.

In some embodiments, the agent is an antisense oligomer (ASO) complementary to the targeted region of the pre-mRNA.

In some embodiments, the agent is at least 75%, at least 80%, at least 85%, at least 90%, at least 95%, at least 98%, at least 99%, or 100%, complementary to the targeted region of the pre-mRNA encoding the target protein.

In some embodiments, the agent comprises a backbone modification comprising a phosphorothioate linkage or a phosphorodiamidate linkage.

In some embodiments, the agent comprises a phosphorodiamidate morpholino.

In some embodiments, the agent comprises a locked nucleic acid.

In some embodiments, the agent comprises a peptide nucleic acid.

In some embodiments, the agent comprises a 2′-O-methyl.

In some embodiments, the agent comprises a 2′-Fluoro, or a 2′-O-methoxyethyl moiety.

In some embodiments, the agent comprises at least one modified sugar moiety.

In some embodiments, each sugar moiety is a modified sugar moiety.

In some embodiments, the agent is an antisense oligomer, and wherein the agent consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

In some embodiments, the composition comprises a vector encoding the agent.

In some embodiments, the agent is a polynucleotide comprising an antisense oligomer.

In some embodiments, the vector is a viral vector.

In some embodiments, the viral vector is an adenovirus-associated viral vector.

In some embodiments, the polynucleotide further comprises a modified snRNA.

In some embodiments, the modified human snRNA is a modified U1 snRNA or a modified U7 snRNA.

In some embodiments, the modified human snRNA is a modified U7 snRNA and wherein the antisense oligomer has a sequence at least 75%, at least 80%, at least 85%, at least 90%, at least 95%, at least 98%, at least 99%, or 100% identical to a sequence listed in Table 4 or Table 5.

Described herein, in certain embodiments, is a composition comprising a nucleic acid molecule that encodes for the agent according to the composition as described herein.

In some embodiments, the nucleic acid molecule is incorporated into a viral delivery system.

In some embodiments, the viral delivery system is an adenovirus-associated vector.

In some embodiments, the viral vector is an adenovirus-associated viral vector.

Described herein, in certain embodiments, is a method of modulating expression of a target protein in a cell comprising a pre-mRNA that is transcribed from a target gene and encodes the target protein, the method comprising: contacting a non-sense mediated RNA decay alternative splice site (NSASS) modulating agent or a viral vector encoding the agent to the cell, wherein the pre-mRNA comprises an alternative 5′ splice-site downstream of a canonical 5′ splice-site, wherein a processed mRNA that is produced by splicing of the pre-mRNA at the alternative 5′ splice-site undergoes non-sense mediated RNA decay, wherein the agent modulates processing of the pre-mRNA by modulating splicing at the alternative 5′ splice-site, thereby modulating expression of the target protein; and wherein the target gene is PKD2.

In some embodiments, the agent: (a) binds to a targeted portion of the pre-mRNA; (b) modulates binding of a factor involved in splicing at the alternative 5′ splice-site; or (c) a combination of (a) and (b).

In some embodiments, the agent interferes with binding of the factor involved in splicing at the alternative 5′ splice-site to a region of the targeted portion.

In some embodiments, the targeted portion of the pre-mRNA is proximal to the alternative 5′ splice-site.

In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of the alternative 5′ splice-site.

In some embodiments, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of the alternative 5′ splice-site.

In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of the alternative 5′ splice-site.

In some embodiments, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of the alternative 5′ splice-site.

In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site of GRCh38/hg38: chr4 88036480.

In some embodiments, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site of GRCh38/hg38: chr4:88036480.

In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site of GRCh38/hg38: chr4:88036480.

In some embodiments, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site of GRCh38/hg38: chr4:88036480.

In some embodiments, the targeted portion of the pre-mRNA is located in a region between the canonical 5′ splice-site and the alternative 5′ splice-site.

In some embodiments, the targeted portion of the pre-mRNA is located in an exon region extended by the splicing at the alternative 5′ splice-site.

In some embodiments, the targeted portion of the pre-mRNA at least partially overlaps with the alternative 5′ splice-site.

In some embodiments, the targeted portion of the pre-mRNA at least partially overlaps with a region upstream or downstream of the alternative 5′ splice-site.

In some embodiments, the targeted portion of the pre-mRNA is within an exon region extended by the splicing at the alternative 5′ splice-site.

In some embodiments, the targeted portion of the pre-mRNA comprises about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of an exon region extended by the splicing at the alternative 5′ splice-site.

In some embodiments, the targeted portion of the pre-mRNA is located in an intronic region between two canonical exons.

In some embodiments, the targeted portion of the pre-mRNA is located in one of the two canonical exons.

In some embodiments, the targeted portion of the pre-mRNA is located in a region spanning both an intron and a canonical exon.

In some embodiments, the level the target protein in the cell is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell.

In some embodiments, modulation of splicing of the pre-mRNA increases production of the processed mRNA encoding the target protein.

In some embodiments, the level of processed mRNA encoding the target protein in the cell contacted with the agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell.

In some embodiments, the target protein is the canonical isoform of the protein.

In some embodiments, the processed mRNA that is produced by splicing of the pre-mRNA at the alternative 5′ splice-site comprises a premature termination codon (PTC).

In some embodiments, the NSASS modulating agent is the composition as described herein.

Described herein, in certain embodiments, is a pharmaceutical composition comprising the composition as described herein; and a pharmaceutically acceptable excipient and/or a delivery vehicle.

Described herein, in certain embodiments, is a method of treating or reducing the likelihood of developing a disease or condition in a subject in need thereof, the method comprising: administering to the subject a pharmaceutical composition to a subject in need thereof, wherein the pharmaceutical composition comprises a composition comprising a non-sense mediated RNA decay alternative splice site (NSASS) modulating agent or a viral vector encoding the agent, wherein the agent modulates expression of a target protein in a cell comprising a pre-mRNA that is transcribed from a target gene and encodes the target protein, wherein the pre-mRNA comprises an alternative 5′ splice-site downstream of a canonical 5′ splice-site, wherein splicing of the pre-mRNA at the alternative 5′ splice-site leads to non-sense mediated RNA decay of the alternatively spliced mRNA, wherein the agent modulates processing of the pre-mRNA by modulating splicing at the alternative 5′ splice-sites; and wherein the target gene is PKD2 and a pharmaceutically acceptable excipient.

Described herein, in certain embodiments, is a method of treating or reducing the likelihood of developing a disease or condition in a subject in need thereof, the method comprising: administering to the subject the pharmaceutical composition as described herein.

In some embodiments, the disease is polycystic kidney disease with or without polycystic liver disease, autosomal dominant polycystic kidney disease, or intracranial aneurysm.

In some embodiments, the disease or condition is a disease or condition associated with a deficiency in amount or activity of polycystin 2 or polycystin 1.

In some embodiments, the disease or condition is a disease or condition associated with a deficiency in amount or activity of a protein that polycystin 2 functionally augments, compensates for, replaces or functionally interacts with.

In some embodiments, the disease or the condition is caused by a deficient amount or activity of the target protein.

In some embodiments, the agent increases the level of the processed mRNA encoding the target protein in the cell.

In some embodiments, the level of processed mRNA encoding the target protein in the cell contacted with the agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell.

In some embodiments, the agent increases the expression of the target protein in the cell.

In some embodiments, the level the target protein in the cell is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell.

In some embodiments, the method further comprises assessing mRNA levels or expression levels of the target protein.

In some embodiments, the method further comprises assessing the subject's genome for at least one genetic mutation associated with the disease.

In some embodiments, at least one genetic mutation is within a locus of a gene associated with the disease.

In some embodiments, at least one genetic mutation is within a locus associated with expression of a gene associated with the disease.

In some embodiments, at least one genetic mutation is within the PKD2 gene locus.

In some embodiments, at least one genetic mutation is within a locus associated with PKD2 gene expression.

In some embodiments, the subject is a human.

In some embodiments, the subject is a non-human animal.

In some embodiments, the subject is a fetus, an embryo, or a child.

In some embodiments, the cell or the cells is ex vivo, or in a tissue, or organ ex vivo.

In some embodiments, the agent is administered to the subject by intracerebroventricular injection, intraperitoneal injection, intramuscular injection, intrathecal injection, subcutaneous injection, oral administration, synovial injection, intravitreal administration, subretinal injection, topical application, implantation, or intravenous injection.

In some embodiments, the method treats the disease or condition.

Described herein, in certain embodiments, is a therapeutic agent for use in the method as described herein.

Described herein, in certain embodiments, is a pharmaceutical composition comprising the therapeutic agent as described herein and a pharmaceutically acceptable excipient.

Described herein, in certain embodiments, is a method of treating or reducing the likelihood of developing a disease or condition in a subject in need thereof, comprising: administering the pharmaceutical composition as described herein by intracerebroventricular injection, intraperitoneal injection, intramuscular injection, intrathecal injection, subcutaneous injection, oral administration, synovial injection, intravitreal administration, subretinal injection, topical application, implantation, or intravenous injection to the subject.

In some embodiments, the method treats the subject.

Described herein, in certain embodiments, is a method of increasing expression of a polycystin 2 protein in a cell having a processed mRNA that encodes the polycystin 2 protein and that comprises a translation regulatory element that inhibits translation of the processed mRNA, the method comprising contacting an agent or a vector encoding the agent to the cell, wherein the agent modulates a structure of the translation regulatory element, thereby increasing expression of the polycystin 2 protein in the cell.

Described herein, in certain embodiments, is a method of increasing expression of a polycystin 2 protein in a cell having a processed mRNA that encodes the polycystin 2 protein and that comprises a translation regulatory element that inhibits translation of the processed mRNA, the method comprising contacting an agent or a vector encoding the agent to the cell, wherein the agent (a) binds to a targeted portion of the processed mRNA; (b) modulates interaction of the translation regulatory element with a factor involved in translation of the processed mRNA; or (c) a combination of (a) and (b), thereby increasing expression of the polycystin 2 protein in the cell.

Described herein, in certain embodiments, is a method of modulating expression of an polycystin 2 protein in a cell, the method comprising contacting an agent or a vector encoding the agent to the cell, wherein the agent comprises an antisense oligomer with at least 80% sequence identity to a sequence selected from the group consisting of a sequence of Table 4 or Table 5.

Described herein, in certain embodiments, is a composition comprising an agent or a vector encoding the agent, wherein the agent comprises an antisense oligomer with at least 80% sequence identity to a sequence selected from the group consisting of a sequence of Table 4 or Table 5.

Described herein, in certain embodiments, is a composition comprising a vector encoding an agent, wherein the agent comprises a polynucleic acid comprising a sequence with at least 80% sequence identity to a sequence selected from the group consisting sequence of Table 4 or Table 5.

Described herein, in certain embodiments, is a composition comprising an agent, wherein the agent comprises an antisense oligomer that binds to a targeted portion of a processed mRNA that encodes polycystin 2 protein, wherein the targeted portion of the processed mRNA comprises at least one nucleotide of the main start codon of the processed mRNA or is within the 5′ UTR of the processed mRNA.

Described herein, in certain embodiments, is a composition comprising a vector encoding an agent, wherein the agent comprises a polynucleic acid comprising a sequence that binds to a targeted portion of a processed mRNA that encodes polycystin 2 protein, wherein the targeted portion of the processed mRNA comprises at least one nucleotide of the main start codon of the processed mRNA or is within the 5′ UTR of the processed mRNA.

Described herein, in certain embodiments, is a composition comprising an agent, wherein the agent modulates structure of a translation regulatory element of a processed mRNA that encodes an polycystin 2 protein, thereby increasing expression of the polycystin 2 protein, and wherein the translation regulatory element inhibits translation of the processed mRNA.

Described herein, in certain embodiments, is a composition comprising a vector encoding an agent, wherein the agent modulates structure of a translation regulatory element of a processed mRNA that encodes an polycystin 2 protein, thereby increasing expression of the polycystin 2 protein, and wherein the translation regulatory element inhibits translation of the processed mRNA.

Described herein, in certain embodiments, is a composition comprising an agent, wherein the agent increases translation of a processed mRNA in a cell, wherein the processed mRNA encodes polycystin 2 protein and comprises a translation regulatory element that inhibits the translation of the processed mRNA, wherein the agent modulates a structure of the translation regulatory element, thereby increasing translation efficiency and/or the rate of translation of the processed mRNA, wherein the agent (a) binds to a targeted portion of the processed mRNA; (b) modulates interaction of the translation regulatory element with a factor involved in translation of the processed mRNA; or (c) a combination of (a) and (b).

Described herein, in certain embodiments, is a composition comprising a vector encoding an agent, wherein the agent increases translation of a processed mRNA in a cell, wherein the processed mRNA encodes polycystin 2 protein and comprises a translation regulatory element that inhibits the translation of the processed mRNA, wherein the agent modulates a structure of the translation regulatory element, thereby increasing translation efficiency and/or the rate of translation of the processed mRNA, wherein the agent (a) binds to a targeted portion of the processed mRNA; (b) modulates interaction of the translation regulatory element with a factor involved in translation of the processed mRNA; or (c) a combination of (a) and (b).

Described herein, in certain embodiments, is a method of increasing expression of a target protein in a cell having a processed mRNA that encodes the target protein and comprises a translation regulatory element that inhibits translation of the processed mRNA, the method comprising delivering into the cell: (1) a first agent or a first nucleic acid sequence encoding the first agent, and (2) a second agent or a second nucleic acid sequence encoding the second agent, wherein the first agent modulates splicing of a pre-mRNA that is transcribed from an target gene that encodes the target protein, and wherein the second agent modulates a structure of the translation regulatory element of the processed mRNA that encodes the target protein, thereby increasing the expression of the target protein in the cell, wherein the target protein is polycystin 2.

Described herein, in certain embodiments, is a method of increasing expression of a target protein in a cell having a processed mRNA that encodes the target protein and comprises a translation regulatory element that inhibits translation of the processed mRNA, the method comprising delivering into the cell: (1) a first agent or a first nucleic acid sequence encoding the first agent, and (2) a second agent or a second nucleic acid sequence encoding the second agent, wherein the first agent modulates splicing of a pre-mRNA that is transcribed from an target gene that encodes the target protein, and wherein the second agent (a) binds to a targeted portion of the processed mRNA; (b) modulates interaction of the translation regulatory element with a factor involved in translation of the processed mRNA; or (c) a combination of (a) and (b), thereby increasing the expression of the target protein in the cell, wherein the target protein is polycystin 2. In some embodiments, the agent modulates a structure of the translation regulatory element. In some embodiments, the agent: (a) binds to a targeted portion of the processed mRNA; (b) modulates interaction of the translation regulatory element with a factor involved in translation of the processed mRNA; or (c) a combination of (a) and (b). In some embodiments, the translation regulatory element is in a 5′ untranslated region (5′ UTR) of the processed mRNA. In some embodiments, the translation regulatory element comprises at least a portion of a 5′ UTR of the processed mRNA. In some embodiments, the translation regulatory element comprises a secondary mRNA structure that involves base-pairing with at least one nucleotide of the main start codon of the processed mRNA. In some embodiments, the agent inhibits the base-pairing with the at least one nucleotide of the main start codon of the processed mRNA. In some embodiments, the mRNA secondary structure comprises a stem, a stem loop, a Guanine quadruplex, or any combination thereof. In some embodiments, the agent does not bind to the main start codon. In some embodiments, the agent binds to at least one nucleotide of the main start codon. In some embodiments, the agent inhibits or reduces formation of a secondary mRNA structure comprising the at least one nucleotide of the main start codon of the processed mRNA. In some embodiments, the agent inhibits or reduces base-pairing of the at least one nucleotide of the main start codon of the processed mRNA with another nucleotide of the processed mRNA, optionally wherein the another nucleotide is another nucleotide of the 5′ UTR of the processed mRNA. In some embodiments, the translation regulatory element comprises at least part of an upstream open reading frame (uORF). In some embodiments, the agent promotes formation of a secondary mRNA structure that involves the at least part of the uORF. In some embodiments, the translation regulatory element comprises an upstream start codon. In some embodiments, the agent promotes formation of a secondary mRNA structure that involves base-pairing with at least one nucleotide of the upstream start codon. In some embodiments, the agent does not bind to the upstream start codon. In some embodiments, the agent binds to the upstream start codon. In some embodiments, the agent promotes or increases formation of a secondary mRNA structure comprising the at least one nucleotide of the upstream start codon. In some embodiments, the agent promotes or increases base-pairing of the at least one nucleotide of the upstream start codon with another nucleotide of the processed mRNA, optionally wherein the another nucleotide is another nucleotide of the 5′ UTR of the processed mRNA. In some embodiments, the translation regulatory element comprises a Guanine quadruplex formed by a G-rich sequence of the processed mRNA. In some embodiments, the agent inhibits formation of the Guanine quadruplex. In some embodiments, the G-rich sequence comprises at least a portion of 5′ untranslated region (5′ UTR) of the processed mRNA. In some embodiments, the G-rich sequence is present in 5′ untranslated region (5′ UTR) of the processed mRNA. In some embodiments, the G-rich sequence comprises a sequence according to the formula Gx-N1-7-Gx-N1-7-Gx-N1-7-Gx (SEQ ID NO: 227), where x≥3 and N is A, C, G or U. In some embodiments, the G-rich sequence comprises a sequence GGGAGCCGGGCUGGGGCUCACACGGGGG (SEQ ID NO: 228). In some embodiments, at least one, two, three or all four of the Gx sequences are structured, present in a secondary structure, or base-paired with another nucleotide, optionally wherein the another nucleotide is a C or a U. In some embodiments, the agent relaxes, promotes deformation of, or inhibits or reduces formation of the Guanine quadruplex. In some embodiments, the agent relaxes, promotes deformation, or inhibits or reduces base-pairing or a structure of at least one, two, three or all four of the Gx sequences of the Guanine quadruplex. In some embodiments, the targeted portion of the processed mRNA is within the 5′ UTR of the processed mRNA. In some embodiments, the targeted portion of the processed mRNA has a sequence with at least 80% sequence identity to at least 8 contiguous nucleotides of a sequence selected from the group consisting of a sequence in Table 3. In some embodiments, the targeted portion of the processed mRNA comprises at least one nucleotide upstream of the codon immediately downstream from the main start codon of the processed mRNA. In some embodiments, the targeted portion of the processed mRNA is at least 20, 30, 40, 50, 60, 70, 80, 90, 100, 110, 120, 150, 160, 180, or 200 nucleotides upstream of the main start codon of the processed mRNA. In some embodiments, the targeted portion of the processed mRNA is about 20, 30, 40, 50, 60, 70, 80, 90, 100, 110, 120, 150, 160, 180, 200, or 220 nucleotides upstream of the main start codon. In some embodiments, the processed mRNA has a sequence with at least 80% sequence identity to a sequence selected from the group consisting of a sequence in Table 3. In some embodiments, the agent comprises an antisense oligomer. In some embodiments, the antisense oligomer has at least 80% sequence identity to a sequence selected from the group consisting of a sequence in Table 4 or Table 5. In some embodiments, the translation regulatory element inhibits the translation of the processed mRNA by inhibiting translation efficiency and/or rate of translation of the processed mRNA. In some embodiments, the agent increases the expression of the polycystin 2 protein in the cell by increasing the translation efficiency and/or rate of translation of the processed mRNA. In some embodiments, the antisense oligomer has about 100% sequence identity to a sequence selected from the group consisting of a sequence in Table 4 or Table 5. In some embodiments, the antisense oligomer has at least 80% sequence identity to a sequence selected from the group consisting of a sequence in Table 4 or Table 5. In some embodiments, the antisense oligomer has about 100% sequence identity to a sequence selected from the group consisting of a sequence in Table 4 or Table 5. In some embodiments, the agent modulates binding of one or more factors that regulate translation of the processed mRNA. In some embodiments, the antisense oligomer comprises a backbone modification comprising a phosphorothioate linkage or a phosphorodiamidate linkage. In some embodiments, the antisense oligomer comprises a phosphorodiamidate morpholino, a locked nucleic acid, a peptide nucleic acid, a 2′-O-methyl moiety, a 2′-Fluoro moiety, or a 2′-O-methoxyethyl moiety. In some embodiments, the antisense oligomer comprises at least one modified sugar moiety. In some embodiments, each sugar moiety is a modified sugar moiety. In some embodiments, the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases. In some embodiments, the vector comprises a viral vector encoding the agent. In some embodiments, the viral vector comprises an adenoviral vector, adeno-associated viral (AAV) vector, lentiviral vector, Herpes Simplex Virus (HSV) viral vector, or retroviral vector. In some embodiments, the agent further comprises a cell penetrating peptide. In some embodiments, the agent comprises the cell penetrating peptide conjugated to an antisense oligomer. In some embodiments, the antisense oligomer is a phosphorodiamidate morpholino oligomer. In some embodiments, the agent comprises a gene editing molecule or polynucleotide encoding a genomic editing molecule. In some embodiments, the agent comprises a polynucleic acid polymer that binds to a target motif of (i) the processed mRNA transcript, (ii) a pre-mRNA from which the processed mRNA transcript is processed, or (iii) a gene encoding the pre-mRNA. In some embodiments, the gene editing molecule comprises CRISPR-Cas9 or a functional equivalent thereof, and/or a polynucleic acid polymer that binds to a target motif of (i) the processed mRNA transcript, (ii) a pre-mRNA from which the processed mRNA transcript is processed, or (iii) a gene encoding the pre-mRNA. In some embodiments, the polynucleic acid polymer that binds to a target motif comprises a guide RNA (gRNA). In some embodiments, the agent increases expression of the polycystin 2 protein in the cell. In some embodiments, translation efficiency and/or rate of translation of a processed mRNA that encodes the polycystin 2 protein in the cell is increased. In some embodiments, the translation efficiency and/or rate of translation of the processed mRNA that encodes the polycystin 2 protein in the cell contacted with the agent or the vector encoding the agent is increased compared to the translation efficiency and/or rate of translation of the processed mRNA in a control cell not contacted with the agent or the vector encoding the agent. In some embodiments, the translation efficiency and/or rate of translation of the processed mRNA that encodes the polycystin 2 protein in the cell contacted with the agent or the vector encoding the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the translation efficiency and/or rate of translation of the processed mRNA in a control cell not contacted with the agent or the vector encoding the agent. In some embodiments, the translation efficiency and/or rate of translation of the processed mRNA that encodes the polycystin 2 protein in the cell contacted with the agent or the vector encoding the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of the agent. In some embodiments, a level of the polycystin 2 protein expressed in the cell contacted with the agent or the vector encoding the agent is increased compared to the level of the polycystin 2 protein in a control cell not contacted with the agent or the vector encoding the agent. In some embodiments, a level of the polycystin 2 protein expressed in the cell contacted with the agent or the vector encoding the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of the polycystin 2 protein in a control cell not contacted with the agent or the vector encoding the agent. In some embodiments, a level of the polycystin 2 protein expressed in the cell contacted with the agent or the vector encoding the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of the agent. In some embodiments, the polycystin 2 protein translated from the processed mRNA is a functional polycystin 2 protein. In some embodiments, the polycystin 2 protein is fully functional. In some embodiments, the polycystin 2 protein translated from the processed mRNA is a wild-type polycystin 2 protein. In some embodiments, the polycystin 2 protein translated from the processed mRNA is a full-length polycystin 2 protein. In some embodiments, the processed mRNA transcript is a mutant processed mRNA transcript. In some embodiments, the processed mRNA transcript is not a mutant processed mRNA transcript. In some embodiments, the processed mRNA is processed from a pre-mRNA that is a mutant pre-mRNA. In some embodiments, the processed mRNA is processed from a pre-mRNA that is not a mutant pre-mRNA. In some embodiments, the agent is a therapeutic agent.

In some embodiments, a level of the target protein expressed in the cell is increased by the delivery of (1) the first agent or the first nucleic acid sequence encoding the first agent, and (2) the second agent or the second nucleic acid sequence encoding the second agent. In some embodiments, the level of the target protein expressed in the cell is increased as compared to a control cell. In some embodiments, the control cell is a cell that has not been contacted with the first agent and that has not been contacted with the second agent, or wherein the control cell is a cell to which the first nucleic acid sequence encoding the first agent has not been delivered and to which the second nucleic acid sequence encoding the second agent has not been delivered. In some embodiments, the control cell is a cell that has not been contacted with the first agent and that has been contacted with the second agent, or wherein the control cell is a cell to which the first nucleic acid sequence encoding the first agent has not been delivered and to which the second nucleic acid sequence encoding the second agent has been delivered. In some embodiments, the control cell is a cell that has not been contacted with the second agent and that has been contacted with the first agent, or wherein the control cell is a cell to which the second nucleic acid sequence encoding the second agent has not been delivered and to which the first nucleic acid sequence encoding the first agent has been delivered. In some embodiments, the level of the target protein expressed in the cell is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, as compared to the control cell. In some embodiments, the level of the target protein expressed in the cell, into which (1) the first agent or the first nucleic acid sequence encoding the first agent, and (2) the second agent or the second nucleic acid sequence encoding the second agent, are delivered, is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to in the absence of the first agent or the second agent. In some embodiments, the level of the target protein expressed in the cell, into which (1) the first agent or the first nucleic acid sequence encoding the first agent, and (2) the second agent or the second nucleic acid sequence encoding the second agent, are delivered, is increased by at least about 1.5-fold compared to in the absence of the first agent or the second agent.

Also provided herein is a pharmaceutical composition comprising a therapeutic agent disclosed herein and a pharmaceutically acceptable carrier or excipient.

Also provided herein is a pharmaceutical composition comprising a vector encoding a therapeutic agent disclosed herein and a pharmaceutically acceptable carrier or excipient.

Also provided herein is a pharmaceutical composition comprising a composition disclosed herein and a pharmaceutically acceptable carrier or excipient.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A-FIG. 1B depict a schematic representation of a target pre-mRNA that contains a non-sense mediated mRNA decay-inducing exon (NMD exon) and therapeutic agent-mediated exclusion of the nonsense-mediated mRNA decay-inducing exon to increase expression of functional RNA or the full-length target protein. FIG. 1A shows a cell divided into nuclear and cytoplasmic compartments. In the nucleus, a pre-mRNA transcript of a target gene undergoes splicing to generate mRNA, and this mRNA is exported to the cytoplasm and translated into target protein. For this target gene, some fraction of the mRNA contains a nonsense-mediated mRNA decay-inducing exon (NMD exon mRNA) that is degraded in the cytoplasm, thus leading to no target protein production. FIG. 1B shows an example of the same cell divided into nuclear and cytoplasmic compartments. Treatment with a therapeutic agent, such as an antisense oligomer (ASO), promotes the exclusion of the nonsense-mediated mRNA decay-inducing exon and results in an increase in functional (productive) mRNA, which is in turn translated into higher levels of target protein.

FIG. 2 depicts PKD2 NMD (non-sense mediated mRNA decay)-inducing exon inclusion event. NMD-inducing exon inclusion event: UCSC Genome Browser snapshot of a region in the PKD2 gene (exons are rectangles and introns are lines with arrowheads) that contains an NMD-inducing exon inclusion event (chr4:88031085-88031140) depicted by the shaded area and black bar on the top. RNA sequencing traces from four representative human kidney samples and mixed renal epithelial cells treated with cycloheximide (CHX) or DMSO control are shown.

FIG. 3 depicts PKD2 NMD-inducing alt 5′ss event. NMD-inducing alt 5′ss event: UCSC Genome Browser snapshot of a region in the PKD2 gene (exons are rectangles and introns are lines with arrowheads) that contains an NMD-inducing alternative 5′ splice site event (chr4:88036355-88036480) depicted by the shaded area and black bar on the top. RNA sequencing traces from four representative human kidney samples and mixed renal epithelial cells treated with cycloheximide (CHX) or DMSO control are shown. *alt_5 ss refers to an alternative 5′ splice site.

FIG. 4A-FIG. 4C depicts validation of NMD-inducing events. FIG. 4A shows schematic representation of the NMD-inducing exon (chr4 88031085 88031140) event. FIG. 4B shows schematic representation of the alternative 5′ splice site (Alt 5′ss) (chr4 88036354 88036480) event. *alt_5 ss refers to an alternative 5′ splice site. In this example, the exon resulting from the splicing at the alternative 5′ splice-site (alt 5′ ss) contains an exon region extended by the splicing at the alternative 5′ splice-site (grey bar) in addition to the canonical exon 3 (black bar), and thus, is longer than the corresponding canonical exon 3 (black bar). Consistently, the intron resulting from the splicing at the alternative 5′ splice-site (alt 5′ ss) is shorter than the corresponding canonical intron 3. FIG. 4C shows RT-PCR using RNA from human kidney mixed epithelial cells and human kidney cortical epithelial cells treated with either DMSO (−) or cycloheximide (CHX) (+). Primers were positioned in exon 2 and 4.

FIG. 5 depicts ASO walk design for the NMD exon inclusion event. The shaded nucleotides correspond to the NMD-inducing exon 2×. FIG. 5 discloses SEQ ID NOS 263-265, respectively, in order of appearance.

FIGS. 6A-B depict the ASO walk design for the Alt 5′ss event. The shaded nucleotides correspond to portion of the extended exon 3 that results from the selection of the indicated Alt 5′ss. *alt_5 ss refers to an alternative 5′ splice site. FIG. 6 discloses SEQ ID NOS 266-270, respectively, in order of appearance.

FIG. 7A depicts the changes in productive PKD2 mRNA using primary renal mixed epithelial cells transfected for 24 hours with the indicated ASOs (see macrowalk of FIGS. 6A-B) at 80 nM.

FIG. 7B depicts the changes in nonproductive PKD2 mRNA using primary renal mixed epithelial cells transfected for 24 hours with the indicated ASOs (see macrowalk of FIGS. 6A-B) at 80 nM.

FIG. 8A depicts the changes in productive PKD2 mRNA using primary renal mixed epithelial cells transfected for 24 hours with the indicated ASOs (see macrowalk of FIG. 5) at 80 nM.

FIG. 8B depicts the changes in nonproductive PKD2 mRNA using primary renal mixed epithelial cells transfected for 24 hours with the indicated ASOs (see macrowalk of FIG. 5) at 80 nM.

FIG. 9A-FIG. 9E illustrate ASOs targeting two events to increase productive mRNA and protein production. FIG. 9A depicts the effect of combination of ASOs on non-productive exon inclusion NMD event utilization. FIG. 9B depicts the expression of EX2-EX3 productive mRNA with ASO combinations. FIG. 9C depicts the effect of combination of ASOs on non-productive alternative 5′ss NMD event utilization. FIG. 9D depicts the expression of EX3-EX4 productive mRNA with ASO combinations. FIG. 9E depicts the quantification of Polycystin 2 (PKD2) protein western blots after treatment with ASO combinations.

FIG. 10 illustrated an upstream open reading frame macrowalk of PKD2. Shaded nucleotides correspond to the upstream open reading frame and the canonical start codon, respectively. FIG. 10 discloses SEQ ID NO: 271.

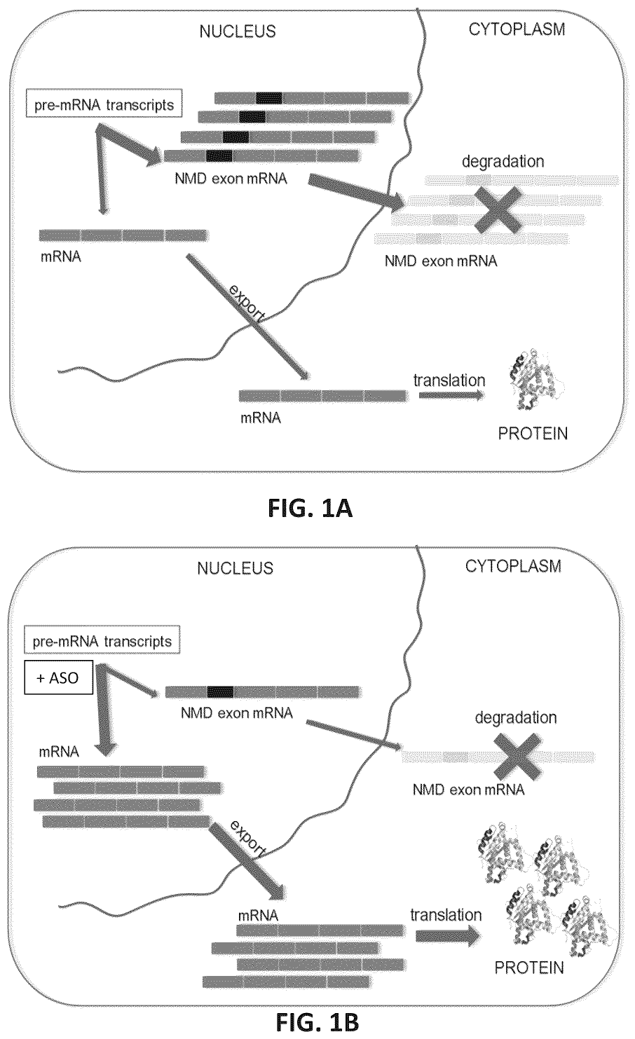

### DETAILED DESCRIPTION

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The coordinate as used herein refers to the coordinate of the genome reference assembly GRCh38 (Genome Research Consortium human build 38), also known as Hg38 (Human genome build 38).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below.

Alternative splicing events in PKD2 gene can lead to non-productive mRNA transcripts which in turn can lead to reduced protein expression, and therapeutic agents which can target the alternative splicing events in PKD2 gene can modulate (e.g., increase) the expression level of functional proteins in patients. Such therapeutic agents can be used to treat a condition caused by polycystin 2 or polycystin 1 deficiency.

One of the alternative splicing events that can lead to non-productive mRNA transcripts is the inclusion of an extra exon in the mRNA transcript that can induce non-sense mediated mRNA decay. The present disclosure also provides compositions and methods for modulating splicing of an extra exon from PKD2 pre-mRNA to increase the production of protein-coding mature mRNA, and thus, translated functional polycystin 2. For example, the compositions and methods provided herein can promote exclusion of an extra exon from PKD2 pre-mRNA, thereby modulating the level of a processed mRNA that is processed from the pre-mRNA and that lacks the extra exon.

Another alternative splicing event that can lead to non-productive mRNA transcripts is an alternative 5′ splice site event. For example, an exon resulting from the splicing at an alternative 5′ splice-site (e.g., downstream of the canonical 5′ ss) can result in a longer exon than the corresponding canonical exon. For example, the intron resulting from the splicing at an alternative 5′ splice-site may be shorter than the corresponding canonical intron. The present disclosure provides compositions and methods for modulating alternative splicing of PKD2 pre-mRNA to increase the production of protein-coding mature mRNA, and thus, translated functional polycystin 2. For example, the compositions and methods provided herein can modulate processing of PKD2 pre-mRNA by preventing or decreasing splicing at the alternative 5′ splice-site.

These compositions and methods include antisense oligomers (ASOs) that can promote constitutive splicing of PKD2 pre-mRNA. In various embodiments, functional polycystin 2 can be increased using the methods of the disclosure to treat a condition caused by polycystin 2 or polycystin 1 deficiency.

“Polycystin 2” also known as APC2, PKD4, Pc-2, TRPP2, Polycystic kidney disease 2, transient receptor potential cation channel, as referred to herein, includes any of the recombinant or naturally-occurring forms of polycystin 2 or variants or homologs thereof that have or maintain polycystin 2 activity (e.g., at least 40% 50%, 60%, 70%, 80%, 90%, 95%, 96%, 97%, 98%, 99% or 100% activity). In some aspects, the variants or homologs have at least 40%, 45%, 50%, 55%, 60%, 65%, 70%, 75%, 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring polycystin 2. In some embodiments, polycystin 2 is substantially identical to the protein identified by the UniProt reference number Q13563 or a variant or homolog having substantial identity thereto.

Autosomal dominant polycystic kidney disease (ADPKD) is a common hereditary disease that accounts for 8-10% of end-stage renal disease. ADPKD is genetically heterogeneous with loci mapped to chromosome 16p13.3 (PKD1) (1) and to chromosome 4q21-23 (PKD2). The predicted structures of polycystin 1 (encoded by PKD1) and polycystin 2 (encoded by PKD2), and their similar disease profiles, suggest their involvement in a common signaling pathway that links extracellular adhesive events to alterations in ion transport. Polycystin 1 and polycystin 2 have been demonstrated to interact through their C-terminal cytoplasmic tails. This interaction resulted in an up-regulation of polycystin 1 but not polycystin 2. The cytoplasmic tail of polycystin 2, but not polycystin 1, formed homodimers through a region distinct from the domain required for interaction with polycystin 1. These results are consistent with a mechanism whereby mutations in polycystin 2 could impede the function of polycystin 1 and thereby result in a disease presentation similar to that of polycystin 1 through distinct molecular lesions of a common signaling pathway operational in normal tubulogenesis. Data supports the idea that polycystin 1 and polycystin 2 participate in a common signaling pathway that prevents cyst formation (Tsiokas et al., PNAS, June 1997, 94 (13) 6965-6970, incorporated herein by reference in its entirety). Data also supports the idea that polycystin 1 and polycystin 2 expression participate in a common signaling pathway that regulates a transition that generally occurs during longer periods of starvation where autophagy is downregulated and cell death increases; polycystin 1 and polycystin 2 can regulate this transition from survival to death starvation in mIMCDs from survival to death (Decuypere, et al., Int. J. Mol. Sci. 2021, 22, 13511). In addition, while it is known that dosage changes in PKD1/PKD2 are important in ADPKD pathogenesis, how PKD1/PKD2 expression is regulated remains poorly understood; although there is evidence of an upstream open reading frame (uORF) in PKD2 that can repress PKD2 translation (Tang, et al., FASEB J. 2013 December; 27(12):4998-5009).

The terms “non-sense mediated RNA decay exon” (or “NSE” or “NMD exon”) and “NMD-inducing exon” (or NIE) are used interchangeably and can refer to an exon (e.g., a noncanonical exon) that can activate the NMD pathway if present in a mature RNA transcript. In constitutive splicing events, the intron containing an NIE is usually spliced out, but the intron or a portion thereof (e.g., NIE) may be retained during alternative or aberrant splicing events. Mature mRNA transcripts containing an NIE may be non-productive, for example, due to frame shifts which induce the NMD pathway. In some embodiments, an NMD exon is an exon that contains a premature stop codon (or premature termination codon (PTC)) or other sequences that facilitate degradation of a mature RNA transcript containing the NMD exon. Inclusion of a NIE in mature RNA transcripts may downregulate gene expression. In some embodiments, an NMD exon is an exon created from alternative splicing events. For example, an NMD exon can be an exon created from an alternative 5′ splice site event. For example, an NMD exon can be an exon that contains a canonical exon and at least a portion of an intron adjacent to the canonical exon. For example, an NMD exon can be an exon that contains an entire canonical exon and at least a portion of an intron immediately downstream of the canonical exon. In some embodiments the NMD exon is a region within an intron (e.g., a canonical intron).

Alternative splicing can result in inclusion of at least one NSE in the mature mRNA transcripts. The terms “mature mRNA,” and “fully-spliced mRNA,” are used interchangeably herein to describe a fully processed mRNA. A mature mRNA that contains an NMD exon can be non-productive mRNA and lead to NMD of the mature mRNA. NIE containing mature mRNA may sometimes lead to reduced protein expression compared to protein expression from a corresponding mature mRNA that does not contain the NIE.

Pseudo splice sites have the same splicing recognition sequences as genuine splice sites but are not used in splicing reactions. They outnumber genuine splice sites in the human genome by an order of a magnitude and are normally repressed by thus far poorly understood molecular mechanisms. Cryptic 5′ splice sites have the consensus NNN/GUNNNN or NNN/GCNNNN where N is any nucleotide and/is the exon-intron boundary. Cryptic 3′ splice sites have the consensus NAG/N. Their activation is positively influenced by surrounding nucleotides that make them more similar to the optimal consensus of authentic splice sites, namely MAG/GURAGU and YAG/G, respectively, where M is C or A, R is G or A, and Y is C or U.

Splice sites and their regulatory sequences can be readily identified by a skilled person using suitable algorithms publicly available, listed for example in Kralovicova, J. and Vorechovsky, I. (2007) Global control of aberrant splice site activation by auxiliary splicing sequences: evidence for a gradient in exon and intron definition. Nucleic Acids Res., 35, 6399-6413, (ncbi.nlm.nih.gov/pmc/articles/PMC2095810/pdf/gkm680.pdf)

Splicing and Nonsense-Mediated mRNA Decay

Intervening sequences or introns are removed by a large and highly dynamic RNA-protein complex termed the spliceosome, which orchestrates complex interactions between primary transcripts, small nuclear RNAs (snRNAs) and a large number of proteins. Spliceosomes assemble ad hoc on each intron in an ordered manner, starting with recognition of the 5′ splice site (5′ss) by U1 snRNA or the 3′splice site (3′ss) by the U2 pathway, which involves binding of the U2 auxiliary factor (U2AF) to the 3′ss region to facilitate U2 binding to the branch point sequence (BPS). U2AF is a stable heterodimer composed of a U2AF2-encoded 65-kD subunit (U2AF65), which binds the polypyrimidine tract (PPT), and a U2AF1-encoded 35-kD subunit (U2AF35), which interacts with highly conserved AG dinucleotides at 3′ss and stabilizes U2AF65 binding. In addition to the BPS/PPT unit and 3′ss/5′ss, accurate splicing requires auxiliary sequences or structures that activate or repress splice site recognition, known as intronic or exonic splicing enhancers or silencers. These elements allow genuine splice sites to be recognized among a vast excess of cryptic or pseudo-sites in the genome of higher eukaryotes, which have the same sequences but outnumber authentic sites by an order of magnitude. Although they often have a regulatory function, the exact mechanisms of their activation or repression are poorly understood.

The decision of whether to splice or not to splice can be typically modeled as a stochastic rather than deterministic process, such that even the most defined splicing signals can sometimes splice incorrectly. However, under normal conditions, pre-mRNA splicing proceeds at surprisingly high fidelity. This is attributed in part to the activity of adjacent cis-acting auxiliary exonic and intronic splicing regulatory elements (ESRs or ISRs). Typically, these functional elements are classified as either exonic or intronic splicing enhancers (ESEs or ISEs) or silencers (ESSs or ISSs) based on their ability to stimulate or inhibit splicing, respectively. Although there is now evidence that some auxiliary cis-acting elements may act by influencing the kinetics of spliceosome assembly, such as the arrangement of the complex between U1 snRNP and the 5′ss, it seems very likely that many elements function in concert with trans-acting RNA-binding proteins (RBPs). For example, the serine- and arginine-rich family of RBPs (SR proteins) is a conserved family of proteins that have a key role in defining exons. SR proteins promote exon recognition by recruiting components of the pre-spliceosome to adjacent splice sites or by antagonizing the effects of ESSs in the vicinity. The repressive effects of ESSs can be mediated by members of the heterogeneous nuclear ribonucleoprotein (hnRNP) family and can alter recruitment of core splicing factors to adjacent splice sites. In addition to their roles in splicing regulation, silencer elements are suggested to have a role in repression of pseudo-exons, sets of decoy intronic splice sites with the typical spacing of an exon but without a functional open reading frame. ESEs and ESSs, in cooperation with their cognate trans-acting RBPs, represent important components in a set of splicing controls that specify how, where and when mRNAs are assembled from their precursors.

The sequences marking the exon-intron boundaries are degenerate signals of varying strengths that can occur at high frequency within human genes. In multi-exon genes, different pairs of splice sites can be linked together in many different combinations, creating a diverse array of transcripts from a single gene. This is commonly referred to as alternative pre-mRNA splicing. Although most mRNA isoforms produced by alternative splicing can be exported from the nucleus and translated into functional polypeptides, different mRNA isoforms from a single gene can vary greatly in their translation efficiency. Those mRNA isoforms with premature termination codons (PTCs) or premature stop codons at least 50 bp upstream of an exon junction complex are likely to be targeted for degradation by the nonsense-mediated mRNA decay (NMD) pathway. Mutations in traditional (BPS/PPT/3′ss/5′ss) and auxiliary splicing motifs can cause aberrant splicing, such as exon skipping or cryptic (or pseudo-) exon inclusion or splice-site activation and contribute significantly to human morbidity and mortality. Both aberrant and alternative splicing patterns can be influenced by natural DNA variants in exons and introns.

Given that exon-intron boundaries can occur at any of the three positions of a codon, it is clear that only a subset of alternative splicing events can maintain the canonical open reading frame. For example, only exons that are evenly divisible by 3 can be skipped or included in the mRNA without any alteration of reading frame. Splicing events that do not have compatible phases will induce a frame-shift. Unless reversed by downstream events, frame-shifts can certainly lead to one or more PTCs, probably resulting in subsequent degradation by NMD. NMD is a translation-coupled mechanism that eliminates mRNAs containing PTCs. NMD can function as a surveillance pathway that exists in all eukaryotes. NMD can reduce errors in gene expression by eliminating mRNA transcripts that contain premature stop codons or PTCs. Translation of these aberrant mRNAs could, in some cases, lead to deleterious gain-of-function or dominant-negative activity of the resulting proteins. NMD targets not only transcripts with PTCs but also a broad array of mRNA isoforms expressed from many endogenous genes, suggesting that NMD is a master regulator that drives both fine and coarse adjustments in steady-state RNA levels in the cell.

### Target Genes

The present disclosure provides compositions and methods for modulating alternative splicing of a target to modulate the production of functional protein-coding mature mRNA, and thus, translated functional the target protein, wherein the target is PKD2. These compositions and methods include antisense oligomers (ASOs) that can promote canonical splicing of the target pre-mRNA, wherein the target is PKD2. In various embodiments, functional target protein can be increased using the methods of the disclosure to treat a condition caused by target protein deficiency, wherein the target is selected from the group consisting of PKD2.

In some embodiments, the methods of the invention are used to increase functional the target protein production to treat a condition in a subject in need thereof, wherein the target is PKD2. In some embodiments, the subject has a condition in which the target protein is not necessarily deficient relative to wild-type, but where an increase in the target protein mitigates the condition nonetheless, wherein the target is PKD2. In some embodiments, the condition is caused by sporadic mutation. In some embodiments, the methods of the invention are used to reduce functional target protein production to treat a condition in a subject in need thereof, wherein the target is PKD2. In some embodiments, the methods of the invention are used to modulate functional target protein production to treat a condition in a subject in need thereof, wherein the target is PKD2.

### Target Transcripts

In some embodiments, the methods of the present disclosure exploit the presence of NIE in the pre-mRNA transcribed from the PKD2 gene. Splicing of the identified PKD2 NIE pre-mRNA species to produce functional mature PKD2 mRNA may be induced using a therapeutic agent such as an ASO that stimulates skipping of an NIE. The resulting mature PKD2 mRNA can be translated normally without activating NMD pathway, thereby increasing the amount of polycystin 2 in the patient's cells and alleviating symptoms of a condition or disease associated with PKD2 deficiency, such as polycystic kidney disease with or without polycystic liver disease, autosomal dominant polycystic kidney disease, and intracranial aneurysm.

Canonical splicing of the identified target NSE pre-mRNA transcripts to produce the functional, mature target mRNA can be induced using a therapeutic agent, such as an ASO, that promotes constitutive splicing of the target NSE pre-mRNA at the canonical splice sites. In some embodiments, the resulting functional, mature target mRNA can be translated normally, thereby increasing the amount of the functional target protein in the patient's cells and preventing symptoms of the target associated disease.

In various embodiments, the present disclosure provides a therapeutic agent that can target PKD2 pre-mRNA to modulate splicing or protein expression level. The therapeutic agent can be a small molecule, nucleic acid oligomer, or polypeptide. In some embodiments, the therapeutic agent is an ASO. Various regions or sequences on the PKD2 pre-mRNA can be targeted by a therapeutic agent, such as an ASO. In some embodiments, the ASO targets a PKD2 NSE pre-mRNA transcribed from the PKD2 gene. In some embodiments, the ASO targets a PKD2 NSE pre-mRNA transcribed from the PKD2 gene comprising non-sense mediated RNA decay exons (NSEs). In some embodiments, the NSE comprises a portion of canonical intron 3 of a PKD2 pre-mRNA transcript (intron downstream of canonical exon 3 of the PKD2 pre-mRNA transcript). In some embodiments, the NSE comprises the entire canonical exon 3 of a PKD2 pre-mRNA transcript. In some embodiments, the NSE comprises only a portion of canonical intron 3 of a PKD2 pre-mRNA transcript and the entire canonical exon 3 of a PKD2 pre-mRNA transcript. In some embodiments, the NSE is included in a PKD2 pre-mRNA transcript due to aberrant splicing. In some embodiments, the ASO targets a sequence within a NSE of a PKD2 pre-mRNA transcript. In some embodiments, the ASO targets a sequence within exon 3 or 4 of a PKD2 pre-mRNA transcript. In some embodiments, the ASO targets an exon sequence upstream (or 5′) from the 5′ splice site of intron 3 following exon 3 of a PKD2 pre-mRNA transcript. In some embodiments, the ASO targets an exon sequence downstream (or 3′) from the 3′ splice site of intron 2 preceding exon 3 of a PKD2 pre-mRNA transcript. In some embodiments, the ASO targets a sequence within an intron flanking the 3′ end of an NSE of a PKD2 pre-mRNA transcript. In some embodiments, the ASO targets a sequence within intron 2 or 3 or 4 of a PKD2 pre-mRNA transcript. In some embodiments, the ASO targets an intron sequence upstream (or 5′) from the 3′ splice site of intron 2 or 3 or 4 of a PKD2 pre-mRNA transcript. In some embodiments, the ASO targets an intron sequence downstream (or 3′) from the 5′ splice site of intron 2 or 3 or 4 of a PKD2 pre-mRNA transcript. In some embodiments, the ASO targets a sequence within an intron flanking the 5′ end of a NSE of a PKD2 pre-mRNA transcript. In some embodiments, the ASO targets a sequence within intron 2 or 3 or 4 of a PKD2 pre-mRNA transcript. In some embodiments, the ASO targets a sequence within intron 2 or 3 or 4 of a PKD2 pre-mRNA transcript. In some embodiments, the ASO targets an intron sequence upstream (or 5′) from the 3′ splice site of intron 2 or 3 or 4 of a PKD2 pre-mRNA transcript. In some embodiments, the ASO targets an intron sequence downstream (or 3′) from the 5′ splice site of intron 2 or 3 or 4 of a PKD2 pre-mRNA transcript. In some embodiments, the ASO targets a sequence comprising an exon-intron boundary of a PKD2 pre-mRNA transcript. In some embodiments, the exon is a NSE. An exon-intron boundary can refer to the junction of an exon sequence and an intron sequence. In some embodiments, the intron sequence can flank the 5′ end of the NSE, or the 3′ end of the exon. In some embodiments, the ASO targets a sequence comprising both a portion of an intron and a portion of an exon.

In some embodiments, the diseases or conditions that can be treated or ameliorated using the method or composition disclosed herein are not directly associated with the target protein (gene) that the therapeutic agent targets. In some embodiments, a therapeutic agent provided herein can target a protein (gene) that is not directly associated with a disease or condition, but the modulation of expression of the target protein (gene) can treat or ameliorate the disease or condition. For instance, targeting genes like PKD2 by a therapeutic agent provided herein can treat or ameliorate polycystic kidney disease with or without polycystic liver disease, autosomal dominant polycystic kidney disease, or intracranial aneurysm. In some embodiments, such target genes like PKD2 are said to be indicated for Pathway (kidney). In some embodiments, such target genes like PKD2 are said to be indicated for Pathway (polycystic kidney disease with or without polycystic liver disease, or autosomal dominant polycystic kidney disease). In some embodiments, such target genes like PKD2 are said to be indicated for Pathway (intracranial aneurysm).

In various embodiments, the present disclosure provides a therapeutic agent which can target PKD2 pre-mRNA transcripts to modulate splicing or protein expression level. The therapeutic agent can be a small molecule, polynucleotide, or polypeptide. In some embodiments, the therapeutic agent is an ASO. Various regions or sequences on the PKD2 pre-mRNA can be targeted by a therapeutic agent, such as an ASO. In some embodiments, the ASO targets a PKD2 pre-mRNA transcript containing an NIE. In some embodiments, the ASO targets a sequence within an NIE of a PKD2 pre-mRNA transcript. In some embodiments, the ASO targets a sequence upstream (or 5′) from the 5′ end of an NIE of a PKD2 pre-mRNA transcript. In some embodiments, the ASO targets a sequence downstream (or 3′) from the 3′ end of an NIE of a PKD2 pre-mRNA transcript. In some embodiments, the ASO targets a sequence that is within an intron flanking the 5′ end of the NIE of a PKD2 pre-mRNA transcript. In some embodiments, the ASO targets a sequence that is within an intron flanking the 3′ end of the NIE of a PKD2 pre-mRNA transcript. In some embodiments, the ASO targets a sequence comprising an NIE-intron boundary of a PKD2 pre-mRNA transcript. An NIE-intron boundary can refer to the junction of an intron sequence and an NIE region. The intron sequence can flank the 5′ end of the NIE, or the 3′ end of the NIE. In some embodiments, the ASO targets a sequence within an exon of a PKD2 pre-mRNA transcript. In some embodiments, the ASO targets a sequence within an intron of a PKD2 pre-mRNA transcript. In some embodiments, the ASO targets a sequence comprising both a portion of an intron and a portion of an exon of a PKD2 pre-mRNA transcript.

In some embodiments, the ASO targets a sequence about 4 to about 300 nucleotides upstream (or 5′) from the 5′ end of the NIE. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides upstream (or 5′) from the 5′ end of the NIE region. In some embodiments, the ASO may target a sequence more than 300 nucleotides upstream from the 5′ end of the NIE. In some embodiments, the ASO targets a sequence about 4 to about 300 nucleotides downstream (or 3′) from the 3′ end of the NIE. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides downstream from the 3′ end of the NIE. In some embodiments, the ASO targets a sequence more than 300 nucleotides downstream from the 3′ end of the NIE.

In some embodiments, the ASOs disclosed herein target a NSE pre-mRNA transcribed from PKD2 genomic sequence. In some embodiments, the ASO targets a NSE pre-mRNA transcript from a genomic sequence comprising a NSE of PKD2 genomic sequences. In some embodiments, the ASO targets a NSE pre-mRNA transcript from a genomic sequence comprising an intron flanking the 3′ end of the NSE and an intron flanking the 5′ end of a NSE of PKD2 genomic sequences. In some embodiments, the ASO targets a NSE pre-mRNA transcript comprising a sequence selected from the group consisting of the pre-mRNA transcripts of Table 3. In some embodiments, the ASO targets a pre-mRNA sequence comprising a NSE of PKD2 pre-mRNA sequences. In some embodiments, the ASO targets a pre-mRNA sequence comprising an intron flanking the 3′ end of the NSE of PKD2 pre-mRNA sequences. In some embodiments, the ASO targets a pre-mRNA sequence comprising an intron flanking the 5′ end of the NSE of PKD2 pre-mRNA sequences. In some embodiments, the transcript is selected from the group consisting of the transcripts of Table 3.

In some embodiments, the PKD2 NIE containing pre-mRNA transcript is encoded by a genetic sequence with at least about 80%, 85%, 90%, 95%, 96%, 97%, 98%, 99% or 100% sequence identity to any one of the sequences listed in Table 2 or Table 3. In some embodiments, the PKD2 NIE pre-mRNA transcript comprises a sequence with at least about 80%, 85%, 90%, 95%, 96%, 97%, 98%, 99% or 100% sequence identity to any one of the sequences listed in Table 2 or Table 3.

In some embodiments, the PKD2 NIE containing pre-mRNA transcript comprises a sequence with at least about 80%, 85%, 90%, 95%, 97%, or 100% sequence identity to any one of the sequences listed in Table 2 or Table 3. In some embodiments, PKD2 NIE containing pre-mRNA transcript is encoded by a sequence with at least about 80%, 85%, 90%, 95%, 97%, or 100% sequence identity to any one of the sequences listed in Table 2 or Table 3. In some embodiments, the targeted portion of the pre-mRNA comprises a sequence with at least 80%, 85%, 90%, 95%, 97%, or 100% sequence identity to a region comprising at least 8 contiguous nucleic acids of any one of the sequences listed in Table 2 or Table 3.

In some embodiments, the pre-mRNA transcript comprises a sequence with at least about 80%, 85%, 90%, 95%, 96%, 97%, 98%, 99% or 100% sequence identity to a pre-mRNA transcript of PKD2 pre-mRNA transcripts or a complement thereof described herein. In some embodiments, the targeted portion of the pre-mRNA selected from the group consisting of PKD2 pre-mRNAs comprises a sequence with at least 80%, 85%, 90%, 95%, 97%, or 100% sequence identity to a region comprising at least 8 contiguous nucleic acids of a sequence of the pre-mRNA transcripts of Table 2 or Table 3 or complements thereof. In some embodiments, the targeted portion of the pre-mRNA of PKD2 pre-mRNA comprises a sequence that is complementary to at least 8, 9, 10, 11, 12, 13, 14, 15, 16, 17, 18, 19 or 20 contiguous nucleic acids of a sequence of Table 2 or Table 3 or a complement thereof.

In some embodiments, the ASOs disclosed herein target a NSE pre-mRNA transcribed from a PKD2 genomic sequence. In some embodiments, the ASO targets a NSE pre-mRNA transcript from a PKD2 genomic sequence comprising a NSE. In some embodiments the NSE comprises exon 3. In some embodiments the NSE is the third exon of a PKD2 transcript. In some embodiments, the ASO targets a NSE pre-mRNA transcript from a PKD2 genomic sequence comprising exon 3 or 4. In some embodiments, the ASO targets a NSE pre-mRNA transcript from a PKD2 genomic sequence comprising an intron flanking the 3′ end of the NSE and an intron flanking the 5′ end of a NSE. In some embodiments, the intron flanking the 3′ end of the NSE is intron 3 and the intron flanking the 5′ end of a NSE is intron 2. In some embodiments, the ASO targets a NSE pre-mRNA transcript from a PKD2 genomic sequence comprising intron 2, exon 3 and intron 3. In some embodiments, the ASO targets a NSE pre-mRNA transcript comprising exon 3 and exon 4. In some embodiments, the ASO targets a PKD2 pre-mRNA sequence comprising a NSE. In some embodiments, the ASO targets a PKD2 pre-mRNA sequence comprising exon 3. In some embodiments, the ASO targets a PKD2 pre-mRNA sequence comprising an intron flanking the 3′ end of the NSE. In some embodiments, the ASO targets a PKD2 pre-mRNA sequence comprising intron 3. In some embodiments, the ASO targets a PKD2 pre-mRNA sequence comprising an intron flanking the 5′ end of the NSE.

In some embodiments, the PKD2 pre-mRNA transcript is encoded by a genetic sequence with at least about 80%, 85%, 90%, 95%, 96%, 97%, 98%, 99% or 100% sequence identity to the Ensembl reference number ENSG00000118762.8 or a complement thereof. In some embodiments, the PKD2 pre-mRNA transcript comprises a sequence with at least about 80%, 85%, 90%, 95%, 96%, 97%, 98%, 99% or 100% sequence identity to a PKD2 pre-mRNA transcript or a complement thereof described herein.

In some embodiments, the targeted portion of the PKD2 pre-mRNA comprises a sequence with at least 80%, 85%, 90%, 95%, 97%, or 100% sequence identity to a region comprising at least 8 contiguous nucleic acids of a sequence of sequence of Table 3 or complements thereof. In some embodiments, the targeted portion of the PKD2 pre-mRNA comprises a sequence with at least 80%, 85%, 90%, 95%, 97%, or 100% sequence identity to a region comprising at least 8 contiguous nucleic acids of a sequence selected from the group consisting of sequences listed in Table 2 or Table 3 or complements thereof. In some embodiments, the ASO comprises a sequence that is at least about 80%, 85%, 90%, 95%, 97%, or 100% identical to any one the sequences of Table 4 or complements thereof.

In some embodiments, the ASOs disclosed herein target a NSE pre-mRNA transcribed from a target genomic sequence. In some embodiments, the ASO targets a NSE pre-mRNA transcript from a target genomic sequence comprising a NSE. In some embodiments, the ASO targets a NSE pre-mRNA transcript from a target genomic sequence comprising an intron flanking the 3′ end of the NSE and an intron flanking the 5′ end of a NSE. In some embodiments, the ASO targets a NSE pre-mRNA transcript comprising a sequence selected from the pre-mRNA transcript sequences of Table 3. In some embodiments, the ASO targets a NSE pre-mRNA transcript comprising a sequence selected from the pre-mRNA transcript sequences of Table 3 as represented by the Ensembl reference numbers. In some embodiments, the ASO targets a target pre-mRNA sequence comprising a NSE. In some embodiments, the ASO targets a target pre-mRNA sequence comprising an intron flanking the 3′ end of the NSE. In some embodiments, the ASO targets a target pre-mRNA sequence comprising an intron flanking the 5′ end of the NSE. In some embodiments, the transcript is selected from the group consisting of the transcript sequences of Table 3. In some embodiments, the transcript is selected from the group consisting of the transcript sequences of Table 3 as represented by the Ensembl reference numbers.

In some embodiments, the target pre-mRNA transcript is encoded by a genetic sequence with at least about 80%, 85%, 90%, 95%, 96%, 97%, 98%, 99% or 100% sequence identity to the gene sequence as represented by the Ensembl reference number or a complement thereof. In some embodiments, the target pre-mRNA transcript comprises a sequence with at least about 80%, 85%, 90%, 95%, 96%, 97%, 98%, 99% or 100% sequence identity to target pre-mRNA transcript or a complement thereof described herein.

In some embodiments, the targeted portion of the target pre-mRNA comprises a sequence with at least 80%, 85%, 90%, 95%, 97%, or 100% sequence identity to a region comprising at least 8 contiguous nucleic acids of a sequence of Table 2 or a sequence of Table 3 or complements thereof. In some embodiments, the targeted portion of the target pre-mRNA comprises a sequence with at least 80%, 85%, 90%, 95%, 97%, or 100% sequence identity to a region comprising at least 8 contiguous nucleic acids of a sequence of Table 3 as represented by the Ensembl reference numbers or a sequence of Table 2 or complements thereof. In some embodiments, the targeted portion of the target pre-mRNA comprises a sequence that is complementary to at least 8, 9, 10, 11, 12, 13, 14, 15, 16, 17, 18, 19 or 20 contiguous nucleic acids of a sequence of Table 2 or Table 3 or a complement thereof.

In some embodiments, the ASO targets exon 3 of a PKD2 pre-mRNA comprising a NSE. In some embodiments, the ASO targets a sequence about 2 nucleotides downstream (or 3′) from the 5′ splice site of intron 3 to about 4 nucleotides upstream (or 5′) from the 3′ splice site of intron 2. In some embodiments, the ASO targets a sequence about 2 nucleotides upstream (or 5′) from the 5′ splice site of intron 3 to about 4 nucleotides downstream (or 3′) from the 3′ splice site of intron 2. In some embodiments, the ASO has a sequence according to any one of the sequences listed in Table 4 or complements thereof.

In some embodiments, the ASO targets intron 3 of a PKD2 pre-mRNA comprising a NSE. In some embodiments, the ASO targets a sequence about 4 to about 300 nucleotides upstream (or 5′) from the 3′ splice site of intron 3. In some embodiments, the ASO targets a sequence about 4 to about 300 nucleotides downstream (or 3′) from the 3′ splice site of intron 3. In some embodiments, the ASO targets a sequence about 4 to about 300 nucleotides upstream (or 5′) from the 5′ splice site of intron 3. In some embodiments, the ASO targets a sequence about 4 to about 300 nucleotides downstream (or 3′) from the 5′ splice site of intron 3. In some embodiments, the ASO targets a sequence about 6 to about 100 nucleotides upstream (or 5′) from the 3′ splice site of intron 2. In some embodiments, the ASO targets a sequence about 4 to about 300 nucleotides downstream (or 3′) from the 3′ splice site of intron 2. In some embodiments, the ASO targets a sequence about 6 to about 100 nucleotides upstream (or 5′) from the 5′ splice site of intron 2. In some embodiments, the ASO targets a sequence about 4 to about 300 nucleotides downstream (or 3′) from the 5′ splice site of intron 2. In some embodiments, the ASO has a sequence according to any sequence of Table 4 or complements thereof.

In some embodiments, the targeted portion of the PKD2 pre-mRNA is in intron 2, 3, or 4. In some embodiments, the targeted portion of the PKD2 pre-mRNA is in exon 2, 3, 4, or 5. In some embodiments, hybridization of an ASO to the targeted portion of the NSE pre-mRNA results in inclusion of canonical exon 3, and subsequently increases polycystin 2 production. In some embodiments, hybridization of an ASO to the targeted portion of the NSE pre-mRNA results in exclusion of a canonical exon, and subsequently decreases polycystin 2 production. In some embodiments, hybridization of an ASO to the targeted portion of the NSE pre-mRNA results in inclusion or exclusion of a canonical exon, and subsequently modulates polycystin 2 production. In some embodiments, the targeted portion of the PKD2 pre-mRNA is in exon 3 or 4. In some embodiments, the targeted portion of the PKD2 pre-mRNA is in intron 2. In some embodiments, the targeted portion of the PKD2 pre-mRNA is in intron 3.

In some embodiments, the ASO targets exon 2× of a PKD2 NIE containing pre-mRNA comprising NIE exon 2.

In some embodiments, the ASO targets exon (GRCh38/hg38: chr4:88031085 88031140) of PKD2.

In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from the 5′ end of exon 2× of PKD2. In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from GRCh38/hg38: chr4:88031085 88031140 of PKD2.

In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from the 5′ end of exon 2× of PKD2. In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from GRCh38/hg38: chr4:88031085 88031140 of PKD2.

In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from the 3′ end of exon 2× of PKD2. In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from GRCh38/hg38: chr4:88031085 88031140 of PKD2.

In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from the 3′ end of exon 2× of PKD2. In some embodiments, the ASO targets a sequence at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from GRCh38/hg38: chr4:88031085 88031140 of PKD2.

In some embodiments, the ASO has a sequence complementary to the targeted portion of the pre-mRNA according to any one of the sequences listed in Table 2 or Table 3.

In some embodiments, the ASO targets a sequence upstream from the 5′ end of an NIE. For example, ASOs targeting a sequence upstream from the 5′ end of an NIE (e.g., exon 2× of PKD2) comprises a sequence that is at least about 80%, 85%, 90%, 95%, 97%, or 100% complimentary to at least 8 contiguous nucleic acids of any one of the sequences listed in Table 2 or Table 3. For example, ASOs targeting a sequence upstream from the 5′ end of an NIE (e.g., exon (GRCh38/hg38: chr4:88031085 88031140) of PKD2) can comprise a sequence with at least 80%, 85%, 90%, 95%, 97%, or 100% sequence identity to any one of the sequences listed in Table 2 or Table 3.

In some embodiments, the ASOs target a sequence containing an exon-intron boundary (or junction). For example, ASOs targeting a sequence containing an exon-intron boundary can comprise a sequence that is at least about 80%, 85%, 90%, 95%, 97%, or 100% complimentary to at least 8 contiguous nucleic acids of any one of the sequences listed in Table 2 or Table 3. In some embodiments, the ASOs target a sequence downstream from the 3′ end of an NIE. For example, ASOs targeting a sequence downstream from the 3′ end of an NIE (e.g., exon 2× of PKD2) can comprise a sequence with at least 80%, 85%, 90%, 95%, 97%, or 100% sequence identity to any one of the sequences listed in Table 2 or Table 3. For example, ASOs targeting a sequence downstream from the 3′ end of an NIE (e.g., exon (GRCh38/hg38: chr4:88031085 88031140) of PKD2) can comprise a sequence with at least 80%, 85%, 90%, 95%, 97%, or 100% sequence identity to any one of the sequences listed in Table 2 or Table 3. In some embodiments, ASOs target a sequence within an NIE.

In some embodiments, the ASO targets exon 2× of a PKD2 NIE containing pre-mRNA comprising NIE exon 2. In some embodiments, the ASO targets a sequence downstream (or 3′) from the 5′ end of exon 2× of PKD2 pre-mRNA. In some embodiments, the ASO targets an exon 2× sequence upstream (or 5′) from the 3′ end of exon 2× of PKD2 pre-mRNA.

In some embodiments, the targeted portion of the PKD2 NIE containing pre-mRNA is in intron 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments, hybridization of an ASO to the targeted portion of the NIE pre-mRNA results in exon skipping of at least one of NIE within intron 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, and subsequently increases polycystin 2 production. In some embodiments, the targeted portion of the PKD2 NIE containing pre-mRNA is in intron 2 of PKD2. In some embodiments, the targeted portion of the PKD2 NIE containing pre-mRNA is intron (GRCh38/hg38:chr4 88019572 88036219) of PKD2.

In some embodiments, the methods and compositions of the present disclosure are used to increase the expression of PKD2 by inducing exon skipping of a NIE of an PKD2 NIE containing pre-mRNA. In some embodiments, the NIE is a sequence within any of introns 1-50. In some embodiments, the NIE is a sequence within any of introns 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. In some embodiments, the NIE can be any PKD2 intron or a portion thereof. In some embodiments, the NIE is within intron 2 of PKD2. In some embodiments, the NIE is within intron (GRCh38/hg38:chr4 88019572 88036219) of PKD2.

### Protein Expression

In some embodiments, a mutation occurs in both alleles. In some embodiments, a mutation occurs in one of the two alleles. In some embodiments, additional mutation occurs in one of the two alleles. In some embodiments, the additional mutation occurs in the same allele as the first mutation. In other embodiments, the additional mutation occurs is a trans mutation.

In some embodiments, the methods described herein are used to increase the production of a functional polycystin 2 protein or RNA. As used herein, the term “functional” refers to the amount of activity or function of a polycystin 2 protein or RNA that is necessary to eliminate any one or more symptoms of a treated condition or disease, e.g., polycystic kidney disease with or without polycystic liver disease, autosomal dominant polycystic kidney disease, and intracranial aneurysm. In some embodiments, the methods are used to increase the production of a partially functional polycystin 2 protein or RNA. As used herein, the term “partially functional” refers to any amount of activity or function of polycystin 2 protein or RNA that is less than the amount of activity or function that is necessary to eliminate or prevent any one or more symptoms of a disease or condition. In some embodiments, a partially functional protein or RNA will have at least 10%, at least 20%, at least 30%, at least 40%, at least 50%, at least 60%, at least 70%, at least 75%, at least 80%, at least 85%, at least 90%, or at least 95% less activity relative to the fully functional protein or RNA.

In some embodiments, the method is a method of increasing the expression of polycystin 2 by cells of a subject having a NIE containing pre-mRNA encoding polycystin 2, wherein the subject has polycystic kidney disease with or without polycystic liver disease, autosomal dominant polycystic kidney disease, or intracranial aneurysm caused by a deficient amount of activity of polycystin 2, and wherein the deficient amount of polycystin 2 is caused by haploinsufficiency of polycystin 2. In such an embodiment, the subject has a first allele encoding functional polycystin 2, and a second allele from which polycystin 2 is not produced. In another such embodiment, the subject has a first allele encoding functional polycystin 2, and a second allele encoding nonfunctional polycystin 2. In another such embodiment, the subject has a first allele encoding functional polycystin 2, and a second allele encoding partially functional polycystin 2. In any of these embodiments, the antisense oligomer binds to a targeted portion of the NIE containing pre-mRNA transcribed from the second allele, thereby inducing exon skipping of the NIE from the pre-mRNA and causing an increase in the level of mature mRNA encoding functional polycystin 2, and an increase in the expression of polycystin 2 in the cells of the subject.

In some embodiments, the method is a method of decreasing the expression of the target protein by cells of a subject having a NSE pre-mRNA encoding the target protein, wherein the subject has a disease caused by an excess amount of activity of the target protein, wherein the excess amount of the target protein is caused by a mutation, and wherein the target is any one selected from the group consisting of PKD2. In some embodiments, the antisense oligomer binds to a targeted portion of the NSE pre-mRNA transcribed from the allele carrying a mutation, thereby increasing alternate splicing of NSEs into the pre-mRNA, and causing an decrease in the level of mature mRNA encoding the functional target protein, and an decrease in the expression of the target protein in the cells of the subject. In related embodiments, the method is a method of using an ASO to decrease the expression of a functional protein or functional RNA. In some embodiments, an ASO is used to decrease the expression of the target protein in cells of a subject having a NSE pre-mRNA encoding the target protein, wherein the subject has an excess in the amount or function of the target protein.

In some embodiments, the method is a method of modulating the expression of the target protein by cells of a subject having a NSE pre-mRNA encoding the target protein, wherein the subject has a disease caused by a deficient or excess amount of activity of the target protein, wherein the deficient or excess amount of the target protein is caused by a mutation, and wherein the target is any one selected from the group consisting of PKD2. In some embodiments, the antisense oligomer binds to a targeted portion of the NSE pre-mRNA transcribed from the allele carrying a mutation, thereby modulating alternate splicing of NSEs into the pre-mRNA, and causing an modulation in the level of mature mRNA encoding the functional target protein, and an modulation in the expression of the target protein in the cells of the subject. In related embodiments, the method is a method of using an ASO to modulate the expression of a functional protein or functional RNA. In some embodiments, an ASO is used to modulate the expression of the target protein in cells of a subject having a NSE pre-mRNA encoding the target protein, wherein the subject has an abnormality in the amount or function of the target protein.

In some embodiments, the method is a method of increasing the expression of polycystin 2 by cells of a subject having a NIE containing pre-mRNA encoding polycystin 2, wherein the subject has polycystic kidney disease with or without polycystic liver disease, autosomal dominant polycystic kidney disease, or intracranial aneurysm caused by a deficient amount of activity of polycystin 2, and wherein the deficient amount of polycystin 2 is caused by autosomal recessive inheritance.

In some embodiments, the method is a method of increasing the expression of polycystin 2 by cells of a subject having a NIE containing pre-mRNA encoding polycystin 2, wherein the subject has polycystic kidney disease with or without polycystic liver disease, autosomal dominant polycystic kidney disease, or intracranial aneurysm caused by a deficient amount of activity of polycystin 2, and wherein the deficient amount of polycystin 2 is caused by autosomal dominant inheritance.

In some embodiments, the method is a method of increasing the expression of polycystin 2 by cells of a subject having a NIE containing pre-mRNA encoding polycystin 2, wherein the subject has polycystic kidney disease with or without polycystic liver disease, autosomal dominant polycystic kidney disease, or intracranial aneurysm caused by a deficient amount of activity of polycystin 2, and wherein the deficient amount of polycystin 2 is caused by X-linked dominant inheritance.

In related embodiments, the method is a method of using an ASO to increase the expression of a protein or functional RNA. In some embodiments, an ASO may be used to increase the expression of polycystin 2 in cells of a subject having a NIE containing pre-mRNA encoding polycystin 2, wherein the subject has a deficiency, e.g., polycystic kidney disease with or without polycystic liver disease, autosomal dominant polycystic kidney disease, or intracranial aneurysm, in the amount or function of a polycystin 2.

In some embodiments, the NIE containing pre-mRNA transcript that encodes the protein that is causative of the disease or condition is targeted by the ASOs described herein. In some embodiments, a NIE containing pre-mRNA transcript that encodes a protein that is not causative of the disease is targeted by the ASOs. For example, a disease that is the result of a mutation or deficiency of a first protein in a particular pathway may be ameliorated by targeting a NIE containing pre-mRNA that encodes a second protein, thereby increasing production of the second protein. In some embodiments, the function of the second protein is able to compensate for the mutation or deficiency of the first protein (which is causative of the disease or condition). In some embodiments, the subject has (a) a first allele that is wild type and (b) a second allele that is a mutant allele from which (i) polycystin 2 is produced at a reduced level compared to production from a wild-type allele, (ii) polycystin 2 is produced in a form having reduced function compared to an equivalent wild-type protein, or (iii) polycystin 2 or functional RNA is not produced.

In some embodiments, the subject has:
(a) a first mutant allele from which
   (i) polycystin 2 is produced at a reduced level compared to production from a wild-type allele,
   (ii) polycystin 2 is produced in a form having reduced function compared to an equivalent wild-type protein, or
   (iii) polycystin 2 or functional RNA is not produced; and
(b) a second mutant allele from which
   (i) polycystin 2 is produced at a reduced level compared to production from a wild-type allele,
   (ii) polycystin 2 is produced in a form having reduced function compared to an equivalent wild-type protein, or
   (iii) polycystin 2 is not produced, and
      wherein the NIE containing pre-mRNA is transcribed from the first allele and/or the second allele, and wherein when the subject has the first mutant allele (a)(iii) the second mutant allele is not (b)(iii), and wherein when the subject has the second mutant allele (b)(iii) the first mutant allele is not (a)(iii). In these embodiments, the ASO binds to a targeted portion of the NIE containing pre-mRNA transcribed from the first allele or the second allele, thereby inducing exon skipping of the NIE from the NIE containing pre-mRNA and causing an increase in the level of mRNA encoding polycystin 2 and an increase in the expression of the target protein or functional RNA in the cells of the subject. In these embodiments, the target protein or functional RNA having an increase in expression level resulting from the exon skipping of the NIE from the NIE containing pre-mRNA may be either in a form having reduced function compared to the equivalent wild-type protein (partially-functional), or having full function compared to the equivalent wild-type protein (fully-functional).

In some embodiments, the level of mRNA encoding polycystin 2 is increased 1.1 to 10-fold, when compared to the amount of mRNA encoding polycystin 2 that is produced in a control cell, e.g., one that is not treated with the antisense oligomer or one that is treated with an antisense oligomer that does not bind to the targeted portion of the PKD2 NIE containing pre-mRNA.

In some embodiments, a subject treated using the methods of the present disclosure expresses a partially functional polycystin 2 from one allele, wherein the partially functional polycystin 2 may be caused by a frameshift mutation, a nonsense mutation, a missense mutation, or a partial gene deletion. In some embodiments, a subject treated using the methods of the disclosure expresses a nonfunctional polycystin 2 from one allele, wherein the nonfunctional polycystin 2 may be caused by a frameshift mutation, a nonsense mutation, a missense mutation, a partial gene deletion, in one allele. In some embodiments, a subject treated using the methods of the disclosure has a PKD2 whole gene deletion, in one allele.

### Exon Inclusion

In some embodiments, the included NIE is the most abundant NIE in a population of NIE containing pre-mRNAs transcribed from the gene encoding the target protein in a cell. In some embodiments, the included NIE is the most abundant NIE in a population of NIE containing pre-mRNAs transcribed from the gene encoding the target protein in a cell, wherein the population of NIE containing pre-mRNAs comprises two or more included NIEs. In some embodiments, an antisense oligomer targeted to the most abundant NIE in the population of NIE containing pre-mRNAs encoding the target protein induces exon skipping of one or two or more NIEs in the population, including the NIE to which the antisense oligomer is targeted or binds. In some embodiments, the targeted region is in a NIE that is the most abundant NIE in a NIE containing pre-mRNA encoding polycystin 2.

The degree of exon inclusion can be expressed as percent exon inclusion, e.g., the percentage of transcripts in which a given NIE is included. In brief, percent exon inclusion can be calculated as the percentage of the amount of RNA transcripts with the exon inclusion, over the sum of the average of the amount of RNA transcripts with exon inclusion plus the average of the amount of RNA transcripts with exon exclusion.

A NSE can be created as a result of splicing in additional base pairs.

The degree of alternative splicing can be expressed as percent alternative splicing, e.g., the percent-age of transcripts in which a given NSE is included. In brief, percent alternative splicing can be calculated as the percentage of the amount of RNA transcripts with the NSE, over the sum of the average of the amount of RNA transcripts with a NSE plus the average of the amount of RNA transcripts with only the canonical exons.

In some embodiments, an included NIE is an exon that is identified as an included NIE based on a determination of at least about 5%, at least about 10%, at least about 15%, at least about 20%, at least about 25%, at least about 30%, at least about 35%, at least about 40%, at least about 45%, or at least about 50%, inclusion. In embodiments, a included NIE is an exon that is identified as a included NIE based on a determination of about 5% to about 100%, about 5% to about 95%, about 5% to about 90%, about 5% to about 85%, about 5% to about 80%, about 5% to about 75%, about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 15%, about 10% to about 100%, about 10% to about 95%, about 10% to about 90%, about 10% to about 85%, about 10% to about 80%, about 10% to about 75%, about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 15% to about 100%, about 15% to about 95%, about 15% to about 90%, about 15% to about 85%, about 15% to about 80%, about 15% to about 75%, about 15% to about 70%, about 15% to about 65%, about 15% to about 60%, about 15% to about 55%, about 15% to about 50%, about 15% to about 45%, about 15% to about 40%, about 15% to about 35%, about 15% to about 30%, about 15% to about 25%, about 20% to about 100%, about 20% to about 95%, about 20% to about 90%, about 20% to about 85%, about 20% to about 80%, about 20% to about 75%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 25% to about 100%, about 25% to about 95%, about 25% to about 90%, about 25% to about 85%, about 25% to about 80%, about 25% to about 75%, about 25% to about 70%, about 25% to about 65%, about 25% to about 60%, about 25% to about 55%, about 25% to about 50%, about 25% to about 45%, about 25% to about 40%, or about 25% to about 35%, inclusion. ENCODE data (described by, e.g., Tilgner, et al., 2012, “Deep sequencing of subcellular RNA fractions shows splicing to be predominantly co-transcriptional in the human genome but inefficient for lncRNAs,” Genome Research 22(9):1616-25) can be used to aid in identifying exon inclusion.

In some embodiments, contacting cells with an ASO that is complementary to a targeted portion of a PKD2 pre-mRNA transcript results in an increase in the amount of polycystin 2 produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of polycystin 2 produced by the cell to which the antisense oligomer is contacted is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of target protein produced by a control compound. In some embodiments, the total amount of polycystin 2 produced by the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, contacting cells with an ASO that is complementary to a targeted portion of a PKD2 pre-mRNA transcript results in an increase in the amount of mRNA encoding PKD2, including the mature mRNA encoding the target protein. In some embodiments, the amount of mRNA encoding polycystin 2, or the mature mRNA encoding polycystin 2, is increased by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of the mRNA encoding polycystin 2, or the mature mRNA encoding polycystin 2 produced in the cell to which the antisense oligomer is contacted is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of mature RNA produced in an untreated cell, e.g., an untreated cell or a cell treated with a control compound. In some embodiments, the total amount of the mRNA encoding polycystin 2, or the mature mRNA encoding polycystin 2 produced in the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold compared to the amount of mature RNA produced in an untreated cell, e.g., an untreated cell or a cell treated with a control compound. A control compound can be, for example, an oligonucleotide that is not complementary to a targeted portion of the PKD2 NIE containing pre-mRNA.

In some embodiments, contacting cells with an ASO that is complementary to a targeted portion of a PKD2 pre-mRNA transcript results in a decrease in the amount of polycystin 2 produced by at least 10, 20, 30, 40, 50, 60, 80, 100%, compared to the amount of the protein produced by a cell in the absence of the ASO/absence of treatment. In some embodiments, the total amount of polycystin 2 produced by the cell to which the antisense oligomer is contacted is decreased about 20% to about 100%, about 50% to about 100%, about 20% to about 50%, or about 20% to about 100% compared to the amount of target protein produced by a control compound. In some embodiments, the total amount of polycystin 2 produced by the cell to which the antisense oligomer is contacted is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, the level of mRNA encoding polycystin 2 is decreased 1.1 to 10-fold, when compared to the amount of mRNA encoding polycystin 2 that is produced in a control cell, e.g., one that is not treated with the antisense oligomer or one that is treated with an antisense oligomer that does not bind to the targeted portion of the PKD2 containing pre-mRNA.

In some embodiments, the level of mRNA encoding polycystin 2 is decreased 1.1 to 10-fold, when compared to the amount of mRNA encoding polycystin 2 that is produced in a control cell, e.g., one that is not treated with the antisense oligomer or one that is treated with an antisense oligomer that does not bind to the targeted portion of the PKD2 pre-mRNA.

In some embodiments of the present invention, a subject can have a mutation in PKD2. A variety of pathogenic variants have been reported to cause PKD2 deficiency, including missense variants, nonsense variants, single- and double-nucleotide insertions and deletions, complex insertion/deletions, and splice site variants. In the presence of this pathogenic variant approximately 2%-5% of transcripts are correctly spliced, allowing for residual enzyme activity. In some embodiments, disease results from loss of function of PKD2 caused by PKD2 pathogenic variants that generate truncated proteins or proteins with altered conformations or reduced activity.

In some embodiments, a subject having any PKD2 mutation known in the art and described as above can be treated using the methods and compositions described herein. In some embodiments, the mutation is within any PKD2 intron or exon. In some embodiments, the mutation is within PKD2 exon 2, 3 or 4.

The NIE can be in any length. In some embodiments, the NIE does not comprise a full sequence of an intron. In some embodiments, the NIE comprises a full sequence of an intron and a full sequence of an exon upstream of the intron and a full sequence of an exon downstream of the intron. In some embodiments, the NIE can be a portion of the intron. In some embodiments, the NIE can comprise a 5′ end portion of a canonical intron sequence. In some embodiments, the NIE can comprise a canonical 5′ss sequence of a canonical intron. In some embodiments, the NIE can comprise a 3′ end portion of a canonical intron. In some embodiments, the NIE can comprise a canonical 3′ss sequence of a canonical intron. In some embodiments, the NIE can be a portion within an intron without inclusion of a canonical 5′ss sequence of the intron. In some embodiments, the NIE can be a portion within an intron without inclusion of a canonical 3′ss sequence of the intron. In some embodiments, the NIE can be a portion within an intron without inclusion of either a canonical 5′ss sequence or a canonical 3′ss sequence of the intron. In some embodiments, the NIE can be from 5 nucleotides to 10 nucleotides in length, from 10 nucleotides to 15 nucleotides in length, from 15 nucleotides to 20 nucleotides in length, from 20 nucleotides to 25 nucleotides in length, from 25 nucleotides to 30 nucleotides in length, from 30 nucleotides to 35 nucleotides in length, from 35 nucleotides to 40 nucleotides in length, from 40 nucleotides to 45 nucleotides in length, from 45 nucleotides to 50 nucleotides in length, from 50 nucleotides to 55 nucleotides in length, from 55 nucleotides to 60 nucleotides in length, from 60 nucleotides to 65 nucleotides in length, from 65 nucleotides to 70 nucleotides in length, from 70 nucleotides to 75 nucleotides in length, from 75 nucleotides to 80 nucleotides in length, from 80 nucleotides to 85 nucleotides in length, from 85 nucleotides to 90 nucleotides in length, from 90 nucleotides to 95 nucleotides in length, or from 95 nucleotides to 100 nucleotides in length. In some embodiments, the NIE can be at least 10 nucleotides, at least 20 nucleotides, at least 30 nucleotides, at least 40 nucleotides, at least 50 nucleotides, at least 60 nucleoids, at least 70 nucleotides, at least 80 nucleotides in length, at least 90 nucleotides, or at least 100 nucleotides in length. In some embodiments, the NIE can be from 100 to 200 nucleotides in length, from 200 to 300 nucleotides in length, from 300 to 400 nucleotides in length, from 400 to 500 nucleotides in length, from 500 to 600 nucleotides in length, from 600 to 700 nucleotides in length, from 700 to 800 nucleotides in length, from 800 to 900 nucleotides in length, from 900 to 1,000 nucleotides in length. In some embodiments, the NIE may be longer than 1,000 nucleotides in length.

Inclusion of a NIE can lead to a frameshift and the introduction of a premature termination codon (PTC) (or premature stop codon)) in the mature mRNA transcript rendering the transcript a target of NMD. Mature mRNA transcript containing NIE can be non-productive mRNA transcript which does not lead to protein expression. The PTC can be present in any position downstream of an NIE. In some embodiments, the PTC can be present in any exon downstream of an NIE. In some embodiments, the PTC can be present within the NIE. For example, inclusion of exon 2× of PKD2 pre-mRNA in an mRNA transcript encoded by the PKD2 gene can induce a PTC in the mRNA transcript. For example, inclusion of exon (GRCh38/hg38: chr4:88031085 88031140) of PKD2 in an mRNA transcript encoded by PKD2.

### Therapeutic Agents

In some embodiments, the agents as used herein refers to the therapeutic agents. In some embodiments, the therapeutic agents as used herein refers to the agents.

In various embodiments of the present disclosure, compositions and methods comprising a therapeutic agent are provided to modulate protein expression level of PKD2. In some embodiments, provided herein are compositions and methods to modulate alternative splicing of PKD2 pre-mRNA. In some embodiments, provided herein are compositions and methods to induce exon skipping in the splicing of PKD2 pre-mRNA, e.g., to induce skipping of a NMD exon during splicing of PKD2 pre-mRNA. In other embodiments, therapeutic agents may be used to induce the inclusion of an exon in order to decrease the protein expression level.

A therapeutic agent disclosed herein can be a NIE repressor agent. A therapeutic agent may comprise a polynucleic acid polymer. A therapeutic agent disclosed herein can be an alternative splicing repressor agent. In some embodiments, a therapeutic agent may comprise a polynucleic acid polymer. In other embodiments, a therapeutic agent may comprise a small molecule. In other embodiments, a therapeutic agent may comprise a polypeptide. In some embodiments, the therapeutic agent is a nucleic acid binding protein, with or without being complexed with a nucleic acid molecule. In other embodiments, the therapeutic agent is a nucleic acid molecule that encodes for another therapeutic agent. In further embodiments, the therapeutic agent is incorporated into a viral delivery system, such as an adenovirus-associated vector.

According to one aspect of the present disclosure, provided herein is a method of treatment or prevention of a condition or disease associated with a functional polycystin 2 or polycystin 1 deficiency, comprising administering a NIE repressor agent to a subject to increase levels of functional polycystin 2, wherein the agent binds to a region of the pre-mRNA transcript to decrease inclusion of the NIE in the mature transcript. For example, provided herein is a method of treatment or prevention of a condition associated with a functional polycystin 2 or polycystin 1 deficiency, comprising administering a NIE repressor agent to a subject to increase levels of functional polycystin 2, wherein the agent binds to a region of an intron containing an NIE (e.g., exon 2× of PKD2) of the pre-mRNA transcript or to a NIE-activating regulatory sequence in the same intron. For example, provided herein is a method of treatment or prevention of a condition associated with a functional polycystin 2 or polycystin 1 deficiency, comprising administering a NIE repressor agent to a subject to increase levels of functional polycystin 2, wherein the agent binds to a region of an intron containing an NIE (e.g., exon (GRCh38/hg38: chr4:88031085 88031140) of PKD2) of the pre-mRNA transcript or to a NIE-activating regulatory sequence in the same intron. For another example, provided herein is a method of treatment or prevention of a condition associated with a functional-polycystin 2 or polycystin 1 deficiency, comprising administering an alternative splicing repressor agent to a subject to increase levels of functional polycystin 2, wherein the agent binds to a region of an exon or an intron (e.g., exon 3 or 4, intron 2, 3 or 4 in human PKD2 gene) of the pre-mRNA transcript.

According to one aspect of the present disclosure, provided herein is a method of treatment or prevention of a condition associated with a functional-polycystin 2 or polycystin 1 deficiency, comprising administering an alternative splicing repressor agent to a subject to increase levels of functional polycystin 2, wherein the agent binds to a region of the pre-mRNA transcript to decrease inclusion of the NSE in the mature transcript.

Alternatively, for example, provided herein is a method of treatment or prevention of a condition associated with a functional target protein overexpression, comprising administering an alternative splicing repressor agent to a subject to decrease levels of functional target protein, wherein the agent binds to a region of an exon or an intron of the pre-mRNA transcript, wherein the target protein is polycystin 2.

Where reference is made to reducing NIE inclusion in the mature mRNA, the reduction may be complete, e.g., 100%, or may be partial. The reduction may be clinically significant. The reduction/correction may be relative to the level of NIE inclusion in the subject without treatment, or relative to the amount of NIE inclusion in a population of similar subjects. The reduction/correction may be at least 10% less NIE inclusion relative to the average subject, or the subject prior to treatment. The reduction may be at least 20% less NIE inclusion relative to an average subject, or the subject prior to treatment. The reduction may be at least 40% less NIE inclusion relative to an average subject, or the subject prior to treatment. The reduction may be at least 50% less NIE inclusion relative to an average subject, or the subject prior to treatment. The reduction may be at least 60% less NIE inclusion relative to an average subject, or the subject prior to treatment. The reduction may be at least 80% less NIE inclusion relative to an average subject, or the subject prior to treatment. The reduction may be at least 90% less NIE inclusion relative to an average subject, or the subject prior to treatment.

Where reference is made to increasing active polycystin 2 levels, the increase may be clinically significant. The increase may be relative to the level of active polycystin 2 in the subject without treatment, or relative to the amount of active polycystin 2 in a population of similar subjects. The increase may be at least 10% more active polycystin 2 relative to the average subject, or the subject prior to treatment. The increase may be at least 20% more active polycystin 2 relative to the average subject, or the subject prior to treatment. The increase may be at least 40% more active polycystin 2 relative to the average subject, or the subject prior to treatment. The increase may be at least 50% more active polycystin 2 relative to the average subject, or the subject prior to treatment. The increase may be at least 80% more active polycystin 2 relative to the average subject, or the subject prior to treatment. The increase may be at least 100% more active polycystin 2 relative to the average subject, or the subject prior to treatment. The increase may be at least 200% more active polycystin 2 relative to the average subject, or the subject prior to treatment. The increase may be at least 500% more active polycystin 2 relative to the average subject, or the subject prior to treatment.

Where reference is made to decreasing functional-polycystin 2 levels, the decrease may be clinically significant. The decrease may be relative to the level of functional-polycystin 2 in the subject without treatment, or relative to the amount of functional-polycystin 2 in a population of similar subjects. The decrease may be at least 10% less functional-polycystin 2 relative to the average subject, or the subject prior to treatment. The decrease may be at least 20% less functional-polycystin 2 relative to the average subject, or the subject prior to treatment. The decrease may be at least 40% less functional-polycystin 2 relative to the average subject, or the subject prior to treatment. The decrease may be at least 50% less functional-polycystin 2 relative to the average subject, or the subject prior to treatment. The decrease may be at least 80% less functional-polycystin 2 relative to the average subject, or the subject prior to treatment. The decrease may be at least 100% less functional-polycystin 2 relative to the average subject, or the subject prior to treatment.

In embodiments wherein the NIE repressor agent comprises a polynucleic acid polymer, the polynucleic acid polymer may be about 50 nucleotides in length. The polynucleic acid polymer may be about 45 nucleotides in length. The polynucleic acid polymer may be about 40 nucleotides in length. The polynucleic acid polymer may be about 35 nucleotides in length. The polynucleic acid polymer may be about 30 nucleotides in length. The polynucleic acid polymer may be about 24 nucleotides in length. The polynucleic acid polymer may be about 25 nucleotides in length. The polynucleic acid polymer may be about 20 nucleotides in length. The polynucleic acid polymer may be about 19 nucleotides in length. The polynucleic acid polymer may be about 18 nucleotides in length. The polynucleic acid polymer may be about 17 nucleotides in length. The polynucleic acid polymer may be about 16 nucleotides in length. The polynucleic acid polymer may be about 15 nucleotides in length. The polynucleic acid polymer may be about 14 nucleotides in length. The polynucleic acid polymer may be about 13 nucleotides in length. The polynucleic acid polymer may be about 12 nucleotides in length. The polynucleic acid polymer may be about 11 nucleotides in length. The polynucleic acid polymer may be about 10 nucleotides in length. The polynucleic acid polymer may be between about 10 and about 50 nucleotides in length. The polynucleic acid polymer may be between about 10 and about 45 nucleotides in length. The polynucleic acid polymer may be between about 10 and about 40 nucleotides in length. The polynucleic acid polymer may be between about 10 and about 35 nucleotides in length. The polynucleic acid polymer may be between about 10 and about 30 nucleotides in length. The polynucleic acid polymer may be between about 10 and about 25 nucleotides in length. The polynucleic acid polymer may be between about 10 and about 20 nucleotides in length. The polynucleic acid polymer may be between about 15 and about 25 nucleotides in length. The polynucleic acid polymer may be between about 15 and about 30 nucleotides in length. The polynucleic acid polymer may be between about 12 and about 30 nucleotides in length.

In embodiments wherein the alternative splicing repressor agent comprises a polynucleic acid polymer, the polynucleic acid polymer may be about 50 nucleotides in length. In embodiments wherein the alternative splicing modulator agent comprises a polynucleic acid polymer, the polynucleic acid polymer may be about 50 nucleotides in length. The polynucleic acid polymer may be about 45 nucleotides in length. The polynucleic acid polymer may be about 40 nucleotides in length. The polynucleic acid polymer may be about 35 nucleotides in length. The polynucleic acid polymer may be about 30 nucleotides in length. The polynucleic acid polymer may be about 24 nucleotides in length. The polynucleic acid polymer may be about 25 nucleotides in length. The polynucleic acid polymer may be about 20 nucleotides in length. The polynucleic acid polymer may be about 19 nucleotides in length. The polynucleic acid polymer may be about 18 nucleotides in length. The polynucleic acid polymer may be about 17 nucleotides in length. The polynucleic acid polymer may be about 16 nucleotides in length. The polynucleic acid polymer may be about 15 nucleotides in length. The polynucleic acid polymer may be about 14 nucleotides in length. The polynucleic acid polymer may be about 13 nucleotides in length. The polynucleic acid polymer may be about 12 nucleotides in length. The polynucleic acid polymer may be about 11 nucleotides in length. The polynucleic acid polymer may be about 10 nucleotides in length. The polynucleic acid polymer may be between about 10 and about 50 nucleotides in length. The polynucleic acid polymer may be between about 10 and about 45 nucleotides in length. The polynucleic acid polymer may be between about 10 and about 40 nucleotides in length. The polynucleic acid polymer may be between about 10 and about 35 nucleotides in length. The polynucleic acid polymer may be between about 10 and about 30 nucleotides in length. The polynucleic acid polymer may be between about 10 and about 25 nucleotides in length. The polynucleic acid polymer may be between about 10 and about 20 nucleotides in length. The polynucleic acid polymer may be between about 15 and about 25 nucleotides in length. The polynucleic acid polymer may be between about 15 and about 30 nucleotides in length. The polynucleic acid polymer may be between about 12 and about 30 nucleotides in length.

The sequence of the polynucleic acid polymer may be at least 50%, 55%, 60%, 65%, 70%, 75%, 80%, 85%, 90%, 91%, 92%, 93%, 94%, 95%, 96%, 97%, 98%, 99%, or 99.5% complementary to a target sequence of an mRNA transcript, e.g., a partially processed mRNA transcript. The sequence of the polynucleic acid polymer may be 100% complementary to a target sequence of a pre-mRNA transcript.

The sequence of the polynucleic acid polymer may have 4 or fewer mismatches to a target sequence of the pre-mRNA transcript. The sequence of the polynucleic acid polymer may have 3 or fewer mismatches to a target sequence of the pre-mRNA transcript. The sequence of the polynucleic acid polymer may have 2 or fewer mismatches to a target sequence of the pre-mRNA transcript. The sequence of the polynucleic acid polymer may have 1 or fewer mismatches to a target sequence of the pre-mRNA transcript. The sequence of the polynucleic acid polymer may have no mismatches to a target sequence of the pre-mRNA transcript.

The polynucleic acid polymer may specifically hybridize to a target sequence of the pre-mRNA transcript. For example, the polynucleic acid polymer may have 91%, 92%, 93%, 94%, 95%, 96%, 97%, 98%, 99%, 99.5% or 100% sequence complementarity to a target sequence of the pre-mRNA transcript. The hybridization may be under high stringent hybridization conditions.

The polynucleic acid polymer comprises a sequence with at least 50%, 55%, 60%, 65%, 70%, 75%, 80%, 85%, 90%, 91%, 92%, 93%, 94%, 95%, 96%, 97%, 98%, 99%, or 99.5% sequence identity to a sequence selected from the group consisting of the sequences listed in Table 4. The polynucleic acid polymer may comprise a sequence with 100% sequence identity to a sequence selected from the group consisting of the sequences listed in Table 4. The polynucleic acid polymer is a sequence with at least 50%, 55%, 60%, 65%, 70%, 75%, 80%, 85%, 90%, 91%, 92%, 93%, 94%, 95%, 96%, 97%, 98%, 99%, or 99.5% sequence identity to a sequence selected from the group consisting of the sequences listed in Table 4. The polynucleic acid polymer is a sequence with 100% sequence identity to a sequence selected from the group consisting of the sequences listed in Table 4.

Where reference is made to a polynucleic acid polymer sequence, the skilled person will understand that one or more substitutions may be tolerated, optionally two substitutions may be tolerated in the sequence, such that it maintains the ability to hybridize to the target sequence; or where the substitution is in a target sequence, the ability to be recognized as the target sequence. References to sequence identity may be determined by BLAST sequence alignment using standard/default parameters. For example, the sequence may have 99% identity and still function according to the present disclosure. In other embodiments, the sequence may have 98% identity and still function according to the present disclosure. In another embodiment, the sequence may have 95% identity and still function according to the present disclosure. In another embodiment, the sequence may have 90% identity and still function according to the present disclosure.

### Antisense Oligomers

Provided herein is a composition comprising an antisense oligomer that induces exon skipping by binding to a targeted portion of a PKD2 NIE containing pre-mRNA. As used herein, the terms “ASO” and “antisense oligomer” are used interchangeably and refer to an oligomer such as a polynucleotide, comprising nucleobases that hybridizes to a target nucleic acid (e.g., a PKD2 NIE containing pre-mRNA) sequence by Watson-Crick base pairing or wobble base pairing (G-U). The ASO may have exact sequence complementary to the target sequence or near complementarity (e.g., sufficient complementarity to bind the target sequence and enhancing splicing at a splice site). ASOs are designed so that they bind (hybridize) to a target nucleic acid (e.g., a targeted portion of a pre-mRNA transcript) and remain hybridized under physiological conditions. Typically, if they hybridize to a site other than the intended (targeted) nucleic acid sequence, they hybridize to a limited number of sequences that are not a target nucleic acid (to a few sites other than a target nucleic acid). Design of an ASO can take into consideration the occurrence of the nucleic acid sequence of the targeted portion of the pre-mRNA transcript or a sufficiently similar nucleic acid sequence in other locations in the genome or cellular pre-mRNA or transcriptome, such that the likelihood the ASO will bind other sites and cause “off-target” effects is limited. Any antisense oligomers known in the art, for example in PCT Application No. PCT/US2014/054151, published as WO 2015/035091, titled “Reducing Nonsense-Mediated mRNA Decay,” incorporated by reference herein, can be used to practice the methods described herein.

In some embodiments, ASOs “specifically hybridize” to or are “specific” to a target nucleic acid or a targeted portion of a NIE containing pre-mRNA. Typically, such hybridization occurs with a Tₘ substantially greater than 37° C., preferably at least 50° C., and typically between 60° C. to approximately 90° C. Such hybridization preferably corresponds to stringent hybridization conditions. At a given ionic strength and pH, the Tₘ is the temperature at which 50% of a target sequence hybridizes to a complementary oligonucleotide.

Oligomers, such as oligonucleotides, are “complementary” to one another when hybridization occurs in an antiparallel configuration between two single-stranded polynucleotides. A double-stranded polynucleotide can be “complementary” to another polynucleotide if hybridization can occur between one of the strands of the first polynucleotide and the second. Complementarity (the degree to which one polynucleotide is complementary with another) is quantifiable in terms of the proportion (e.g., the percentage) of bases in opposing strands that are expected to form hydrogen bonds with each other, according to generally accepted base-pairing rules. The sequence of an antisense oligomer (ASO) need not be 100% complementary to that of its target nucleic acid to hybridize. In certain embodiments, ASOs can comprise at least 70%, at least 75%, at least 80%, at least 85%, at least 90%, at least 95%, at least 96%, at least 97%, at least 98%, or at least 99% sequence complementarity to a target region within the target nucleic acid sequence to which they are targeted. For example, an ASO in which 18 of 20 nucleobases of the oligomeric compound are complementary to a target region, and would therefore specifically hybridize, would represent 90 percent complementarity. In this example, the remaining non-complementary nucleobases may be clustered together or interspersed with complementary nucleobases and need not be contiguous to each other or to complementary nucleobases. Percent complementarity of an ASO with a region of a target nucleic acid can be determined routinely using BLAST programs (basic local alignment search tools) and PowerBLAST programs known in the art (Altschul, et al., J. Mol. Biol., 1990, 215, 403-410; Zhang and Madden, Genome Res., 1997, 7, 649-656).

An ASO need not hybridize to all nucleobases in a target sequence and the nucleobases to which it does hybridize may be contiguous or noncontiguous. ASOs may hybridize over one or more segments of a pre-mRNA transcript, such that intervening or adjacent segments are not involved in the hybridization event (e.g., a loop structure or hairpin structure may be formed). In certain embodiments, an ASO hybridizes to noncontiguous nucleobases in a target pre-mRNA transcript. For example, an ASO can hybridize to nucleobases in a pre-mRNA transcript that are separated by one or more nucleobase(s) to which the ASO does not hybridize.

The ASOs described herein comprise nucleobases that are complementary to nucleobases present in a target portion of a NIE containing pre-mRNA. The term ASO embodies oligonucleotides and any other oligomeric molecule that comprises nucleobases capable of hybridizing to a complementary nucleobase on a target mRNA but does not comprise a sugar moiety, such as a peptide nucleic acid (PNA). The ASOs may comprise naturally occurring nucleotides, nucleotide analogs, modified nucleotides, or any combination of two or three of the preceding. The term “naturally occurring nucleotides” includes deoxyribonucleotides and ribonucleotides. The term “modified nucleotides” includes nucleotides with modified or substituted sugar groups and/or having a modified backbone. In some embodiments, all of the nucleotides of the ASO are modified nucleotides. Chemical modifications of ASOs or components of ASOs that are compatible with the methods and compositions described herein will be evident to one of skill in the art and can be found, for example, in U.S. Pat. No. 8,258,109 B2, U.S. Pat. No. 5,656,612, U.S. Patent Publication No. 2012/0190728, and Dias and Stein, Mol. Cancer Ther. 2002, 347-355, herein incorporated by reference in their entirety.

One or more nucleobases of an ASO may be any naturally occurring, unmodified nucleobase such as adenine, guanine, cytosine, thymine, and uracil, or any synthetic or modified nucleobase that is sufficiently similar to an unmodified nucleobase such that it is capable of hydrogen bonding with a nucleobase present on a target pre-mRNA. Examples of modified nucleobases include, without limitation, hypoxanthine, xanthine, 7-methylguanine, 5, 6-dihydrouracil, 5-methylcytosine, and 5-hydroxymethylcytosine.

The ASOs described herein also comprise a backbone structure that connects the components of an oligomer. The term “backbone structure” and “oligomer linkages” may be used interchangeably and refer to the connection between monomers of the ASO. In naturally occurring oligonucleotides, the backbone comprises a 3′-5′ phosphodiester linkage connecting sugar moieties of the oligomer. The backbone structure or oligomer linkages of the ASOs described herein may include (but are not limited to) phosphorothioate, phosphorodithioate, phosphoroselenoate, phosphorodiselenoate, phosphoroanilothioate, phosphoranilidate, phosphoramidate, and the like. See, e.g., LaPlanche, et al., Nucleic Acids Res. 14:9081 (1986); Stec, et al., J. Am. Chem. Soc. 106:6077 (1984), Stein, et al., Nucleic Acids Res. 16:3209 (1988), Zon, et al., Anti-Cancer Drug Design 6:539 (1991); Zon, et al., Oligonucleotides and Analogues: A Practical Approach, pp. 87-108 (F. Eckstein, Ed., Oxford University Press, Oxford England (1991)); Stec, et al., U.S. Pat. No. 5,151,510; Uhlmann and Peyman, Chemical Reviews 90:543 (1990). In some embodiments, the backbone structure of the ASO does not contain phosphorous but rather contains peptide bonds, for example in a peptide nucleic acid (PNA), or linking groups including carbamate, amides, and linear and cyclic hydrocarbon groups. In some embodiments, the backbone modification is a phosphorothioate linkage. In some embodiments, the backbone modification is a phosphoramidate linkage.

In some embodiments, the stereochemistry at each of the phosphorus internucleotide linkages of the ASO backbone is random. In some embodiments, the stereochemistry at each of the phosphorus internucleotide linkages of the ASO backbone is controlled and is not random. For example, U.S. Pat. App. Pub. No. 2014/0194610, “Methods for the Synthesis of Functionalized Nucleic Acids,” incorporated herein by reference, describes methods for independently selecting the handedness of chirality at each phosphorous atom in a nucleic acid oligomer. In some embodiments, an ASO used in the methods of the disclosure, including, but not limited to, any of the ASOs set forth herein in Tables 5 and 6, comprises an ASO having phosphorus internucleotide linkages that are not random. In some embodiments, a composition used in the methods of the disclosure comprises a pure diastereomeric ASO. In some embodiments, a composition used in the methods of the disclosure comprises an ASO that has diastereomeric purity of at least about 90%, at least about 91%, at least about 92%, at least about 93%, at least about 94%, at least about 95%, at least about 96%, at least about 97%, at least about 98%, at least about 99%, about 100%, about 90% to about 100%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100%.

In some embodiments, the ASO has a nonrandom mixture of Rp and Sp configurations at its phosphorus internucleotide linkages. For example, it has been suggested that a mix of Rp and Sp is required in antisense oligonucleotides to achieve a balance between good activity and nuclease stability (Wan, et al., 2014, “Synthesis, biophysical properties and biological activity of second-generation antisense oligonucleotides containing chiral phosphorothioate linkages,” Nucleic Acids Research 42(22): 13456-13468, incorporated herein by reference). In some embodiments, an ASO used in the methods of the disclosure, including, but not limited to, any of the ASOs set forth herein in SEQ ID NOs: 60-191, comprises about 5-100% Rp, at least about 5% Rp, at least about 10% Rp, at least about 15% Rp, at least about 20% Rp, at least about 25% Rp, at least about 30% Rp, at least about 35% Rp, at least about 40% Rp, at least about 45% Rp, at least about 50% Rp, at least about 55% Rp, at least about 60% Rp, at least about 65% Rp, at least about 70% Rp, at least about 75% Rp, at least about 80% Rp, at least about 85% Rp, at least about 90% Rp, or at least about 95% Rp, with the remainder Sp, or about 100% Rp. In some embodiments, an ASO used in the methods of the disclosure, including, but not limited to, any of the ASOs set forth herein comprise a sequence with at least about 80%, 85%, 90%, 95%, 97%, or 100% sequence identity to a region comprising at least 8 contiguous nucleic acids of any one of SEQ ID NOs: 60-191, comprises about 10% to about 100% Rp, about 15% to about 100% Rp, about 20% to about 100% Rp, about 25% to about 100% Rp, about 30% to about 100% Rp, about 35% to about 100% Rp, about 40% to about 100% Rp, about 45% to about 100% Rp, about 50% to about 100% Rp, about 55% to about 100% Rp, about 60% to about 100% Rp, about 65% to about 100% Rp, about 70% to about 100% Rp, about 75% to about 100% Rp, about 80% to about 100% Rp, about 85% to about 100% Rp, about 90% to about 100% Rp, or about 95% to about 100% Rp, about 20% to about 80% Rp, about 25% to about 75% Rp, about 30% to about 70% Rp, about 40% to about 60% Rp, or about 45% to about 55% Rp, with the remainder Sp.

In some embodiments, an ASO used in the methods of the disclosure, including, but not limited to, any of the ASOs set forth herein comprise a sequence with at least about 80%, 85%, 90%, 95%, 97%, or 100% sequence identity to a region comprising at least 8 contiguous nucleic acids of any one of SEQ ID NOs: 60-191, comprises about 5-100% Sp, at least about 5% Sp, at least about 10% Sp, at least about 15% Sp, at least about 20% Sp, at least about 25% Sp, at least about 30% Sp, at least about 35% Sp, at least about 40% Sp, at least about 45% Sp, at least about 50% Sp, at least about 55% Sp, at least about 60% Sp, at least about 65% Sp, at least about 70% Sp, at least about 75% Sp, at least about 80% Sp, at least about 85% Sp, at least about 90% Sp, or at least about 95% Sp, with the remainder Rp, or about 100% Sp. In embodiments, an ASO used in the methods of the disclosure, including, but not limited to, any of the ASOs set forth herein comprise a sequence with at least about 80%, 85%, 90%, 95%, 97%, or 100% sequence identity to a region comprising at least 8 contiguous nucleic acids of any one of SEQ ID NOs: 60-191, comprises about 10% to about 100% Sp, about 15% to about 100% Sp, about 20% to about 100% Sp, about 25% to about 100% Sp, about 30% to about 100% Sp, about 35% to about 100% Sp, about 40% to about 100% Sp, about 45% to about 100% Sp, about 50% to about 100% Sp, about 55% to about 100% Sp, about 60% to about 100% Sp, about 65% to about 100% Sp, about 70% to about 100% Sp, about 75% to about 100% Sp, about 80% to about 100% Sp, about 85% to about 100% Sp, about 90% to about 100% Sp, or about 95% to about 100% Sp, about 20% to about 80% Sp, about 25% to about 75% Sp, about 30% to about 70% Sp, about 40% to about 60% Sp, or about 45% to about 55% Sp, with the remainder Rp.

Any of the ASOs described herein may contain a sugar moiety that comprises ribose or deoxyribose, as present in naturally occurring nucleotides, or a modified sugar moiety or sugar analog, including a morpholine ring. Non-limiting examples of modified sugar moieties include 2′ substitutions such as 2′-O-methyl (2′-O-Me), 2′-O-methoxyethyl (2′MOE), 2′-O-aminoethyl, 2′F; N3′->P5′ phosphoramidate, 2′dimethylaminooxyethoxy, 2′dimethylaminoethoxyethoxy, 2′-guanidinium, 2′-O-guanidinium ethyl, carbamate modified sugars, and bicyclic modified sugars. In some embodiments, the sugar moiety modification is selected from 2′-O-Me, 2′F, and 2′MOE. In some embodiments, the sugar moiety modification is an extra bridge bond, such as in a locked nucleic acid (LNA). In some embodiments the sugar analog contains a morpholine ring, such as phosphorodiamidate morpholino (PMO). In some embodiments, the sugar moiety comprises a ribofuransyl or 2′deoxyribofuransyl modification. In some embodiments, the sugar moiety comprises 2′4′-constrained 2′O-methyloxyethyl (cMOE) modifications. In some embodiments, the sugar moiety comprises cEt 2′, 4′ constrained 2′-O ethyl BNA modifications. In some embodiments, the sugar moiety comprises tricycloDNA (tcDNA) modifications. In some embodiments, the sugar moiety comprises ethylene nucleic acid (ENA) modifications. In some embodiments, the sugar moiety comprises MCE modifications. Modifications are known in the art and described in the literature, e.g., by Jarver, et al., 2014, “A Chemical View of Oligonucleotides for Exon Skipping and Related Drug Applications,” Nucleic Acid Therapeutics 24(1): 37-47, incorporated by reference for this purpose herein.

In some embodiments, each monomer of the ASO is modified in the same way, for example each linkage of the backbone of the ASO comprises a phosphorothioate linkage or each ribose sugar moiety comprises a 2′O-methyl modification. Such modifications that are present on each of the monomer components of an ASO are referred to as “uniform modifications.” In some examples, a combination of different modifications may be desired, for example, an ASO may comprise a combination of phosphorodiamidate linkages and sugar moieties comprising morpholine rings (morpholinos). Combinations of different modifications to an ASO are referred to as “mixed modifications” or “mixed chemistries.”

In some embodiments, the ASO comprises one or more backbone modifications. In some embodiments, the ASO comprises one or more sugar moiety modification. In some embodiments, the ASO comprises one or more backbone modifications and one or more sugar moiety modifications. In some embodiments, the ASO comprises a 2′MOE modification and a phosphorothioate backbone. In some embodiments, the ASO comprises a phosphorodiamidate morpholino (PMO). In some embodiments, the ASO comprises a peptide nucleic acid (PNA). Any of the ASOs or any component of an ASO (e.g., a nucleobase, sugar moiety, backbone) described herein may be modified in order to achieve desired properties or activities of the ASO or reduce undesired properties or activities of the ASO. For example, an ASO or one or more components of any ASO may be modified to enhance binding affinity to a target sequence on a pre-mRNA transcript; reduce binding to any non-target sequence; reduce degradation by cellular nucleases (i.e., RNase H); improve uptake of the ASO into a cell and/or into the nucleus of a cell; alter the pharmacokinetics or pharmacodynamics of the ASO; and/or modulate the half-life of the ASO.

In some embodiments, the ASOs are comprised of 2′-O-(2-methoxyethyl) (MOE) phosphorothioate-modified nucleotides. ASOs comprised of such nucleotides are especially well-suited to the methods disclosed herein; oligomers having such modifications have been shown to have significantly enhanced resistance to nuclease degradation and increased bioavailability, making them suitable, for example, for oral delivery in some embodiments described herein. See e.g., Geary, et al., J Pharmacol Exp Ther. 2001; 296(3):890-7; Geary, et al., J Pharmacol Exp Ther. 2001; 296(3):898-904.

Methods of synthesizing ASOs will be known to one of skill in the art. Alternatively or in addition, ASOs may be obtained from a commercial source.

Unless specified otherwise, the left-hand end of single-stranded nucleic acid (e.g., pre-mRNA transcript, oligonucleotide, ASO, etc.) sequences is the 5′ end and the left-hand direction of single or double-stranded nucleic acid sequences is referred to as the 5′ direction. Similarly, the right-hand end or direction of a nucleic acid sequence (single or double stranded) is the 3′ end or direction. Generally, a region or sequence that is 5′ to a reference point in a nucleic acid is referred to as “upstream,” and a region or sequence that is 3′ to a reference point in a nucleic acid is referred to as “downstream.” Generally, the 5′ direction or end of an mRNA is where the initiation or start codon is located, while the 3′ end or direction is where the termination codon is located. In some aspects, nucleotides that are upstream of a reference point in a nucleic acid may be designated by a negative number, while nucleotides that are downstream of a reference point may be designated by a positive number. For example, a reference point (e.g., an exon-exon junction in mRNA) may be designated as the “zero” site, and a nucleotide that is directly adjacent and upstream of the reference point is designated “minus one,” e.g., “−1,” while a nucleotide that is directly adjacent and downstream of the reference point is designated “plus one,” e.g., “+1.”

In some embodiments, the ASOs are complementary to (and bind to) a targeted portion of a PKD2 NIE containing pre-mRNA that is downstream (in the 3′ direction) of the 5′ splice site of the intron following the included exon in a PKD2 NIE containing pre-mRNA (e.g., the direction designated by positive numbers relative to the 5′ splice site). In some embodiments, the ASOs are complementary to a targeted portion of the PKD2 NIE containing pre-mRNA that is within the region about +1 to about +500 relative to the 5′ splice site of the intron following the included exon. In some embodiments, the ASOs may be complementary to a targeted portion of a PKD2 NIE containing pre-mRNA that is within the region between nucleotides +6 and +40,000 relative to the 5′ splice site of the intron following the included exon. In some aspects, the ASOs are complementary to a targeted portion that is within the region about +1 to about +40,000, about +1 to about +30,000, about +1 to about +20,000, about +1 to about +15,000, about +1 to about +10,000, about +1 to about +5,000, about +1 to about +4,000, about +1 to about +3,000, about +1 to about +2,000, about +1 to about +1,000, about +1 to about +500, about +1 to about +490, about +1 to about +480, about +1 to about +470, about +1 to about +460, about +1 to about +450, about +1 to about +440, about +1 to about +430, about +1 to about +420, about +1 to about +410, about +1 to about +400, about +1 to about +390, about +1 to about +380, about +1 to about +370, about +1 to about +360, about +1 to about +350, about +1 to about +340, about +1 to about +330, about +1 to about +320, about +1 to about +310, about +1 to about +300, about +1 to about +290, about +1 to about +280, about +1 to about +270, about +1 to about +260, about +1 to about +250, about +1 to about +240, about +1 to about +230, about +1 to about +220, about +1 to about +210, about +1 to about +200, about +1 to about +190, about +1 to about +180, about +1 to about +170, about +1 to about +160, about +1 to about +150, about +1 to about +140, about +1 to about +130, about +1 to about +120, about +1 to about +110, about +1 to about +100, about +1 to about +90, about +1 to about +80, about +1 to about +70, about +1 to about +60, about +1 to about +50, about +1 to about +40, about +1 to about +30, or about +1 to about +20 relative to 5′ splice site of the intron following the included exon. In some aspects, the ASOs are complementary to a targeted portion that is within the region from about +1 to about +100, from about +100 to about +200, from about +200 to about +300, from about +300 to about +400, or from about +400 to about +500 relative to 5′ splice site of the intron following the included exon.

In some embodiments, the ASOs are complementary to (and bind to) a targeted portion of a PKD2 NIE containing pre-mRNA that is upstream (in the 5′ direction) of the 5′ splice site of the intron following the included exon in a PKD2 NIE containing pre-mRNA (e.g., the direction designated by negative numbers relative to the 5′ splice site). In some embodiments, the ASOs are complementary to a targeted portion of the PKD2 NIE containing pre-mRNA that is within the region about −4 to about −270 relative to the 5′ splice site of the intron following the included exon. In some embodiments, the ASOs may be complementary to a targeted portion of a PKD2 NIE containing pre-mRNA that is within the region between nucleotides −1 and −40,000 relative to the 5′ splice site of the intron following the included exon. In some aspects, the ASOs are complementary to a targeted portion that is within the region about −1 to about −40,000, about −1 to about −30,000, about −1 to about −20,000, about −1 to about −15,000, about −1 to about −10,000, about −1 to about −5,000, about −1 to about −4,000, about −1 to about −3,000, about −1 to about −2,000, about −1 to about −1,000, about −1 to about −500, about −1 to about −490, about −1 to about −480, about −1 to about −470, about −1 to about −460, about −1 to about −450, about −1 to about −440, about −1 to about −430, about −1 to about −420, about −1 to about −410, about −1 to about −400, about −1 to about −390, about −1 to about −380, about −1 to about −370, about −1 to about −360, about −1 to about −350, about −1 to about −340, about −1 to about −330, about −1 to about −320, about −1 to about −310, about −1 to about −300, about −1 to about −290, about −1 to about −280, about −1 to about −270, about −1 to about −260, about −1 to about −250, about −1 to about −240, about −1 to about −230, about −1 to about −220, about −1 to about −210, about −1 to about −200, about −1 to about −190, about −1 to about −180, about −1 to about −170, about −1 to about −160, about −1 to about −150, about −1 to about −140, about −1 to about −130, about −1 to about −120, about −1 to about −110, about −1 to about −100 about −1 to about −90, about −1 to about −80, about −1 to about −70, about −1 to about −60, about −1 to about −50, about −1 to about −40, about −1 to about −30, or about −1 to about −20 relative to 5′ splice site of the intron following the included exon.

In some embodiments, the ASOs are complementary to a targeted region of a PKD2 NIE containing pre-mRNA that is upstream (in the 5′ direction) of the 3′ splice site of the intron preceding the included exon in a PKD2 NIE containing pre-mRNA (e.g., in the direction designated by negative numbers). In some embodiments, the ASOs are complementary to a targeted portion of the PKD2 NIE containing pre-mRNA that is within the region about −1 to about −500 relative to the 3′ splice site of the intron preceding the included exon. In some embodiments, the ASOs are complementary to a targeted portion of the PKD2 NIE containing pre-mRNA that is within the region −1 to −40,000 relative to the 3′ splice site of the intron preceding the included exon. In some aspects, the ASOs are complementary to a targeted portion that is within the region about −1 to about −40,000, about −1 to about −30,000, −1 to about −20,000, about −1 to about −15,000, about −1 to about −10,000, about −1 to about −5,000, about −1 to about −4,000, about −1 to about −3,000, about −1 to about −2,000, about −1 to about −1,000, about −1 to about −500, about −1 to about −490, about −1 to about −480, about −1 to about −470, about −1 to about −460, about −1 to about −450, about −1 to about −440, about −1 to about −430, about −1 to about −420, about −1 to about −410, about −1 to about −400, about −1 to about −390, about −1 to about −380, about −1 to about −370, about −1 to about −360, about −1 to about −350, about −1 to about −340, about −1 to about −330, about −1 to about −320, about −1 to about −310, about −1 to about −300, about −1 to about −290, about −1 to about −280, about −1 to about −270, about −1 to about −260, about −1 to about −250, about −1 to about −240, about −1 to about −230, about −1 to about −220, about −1 to about −210, about −1 to about −200, about −1 to about −190, about −1 to about −180, about −1 to about −170, about −1 to about −160, about −1 to about −150, about −1 to about −140, about −1 to about −130, about −1 to about −120, about −1 to about −110, about −1 to about −100, about −1 to about −90, about −1 to about −80, about −1 to about −70, about −1 to about −60, about −1 to about −50, about −1 to about −40, about −1 to about −30, or about −1 to about −20 relative to 3′ splice site of the intron preceding the included exon. In some aspects, the ASOs are complementary to a targeted portion that is within the region from about −1 to about −100, from about −100 to about −200, from about −200 to about −300, from about −300 to about −400, or from about −400 to about −500 relative to 3′ splice site of the intron preceding the included exon.

In some embodiments, the ASOs are complementary to a targeted region of a PKD2 NIE containing pre-mRNA that is downstream (in the 3′ direction) of the 3′ splice site of the intron preceding the included exon in a PKD2 NIE containing pre-mRNA (e.g., in the direction designated by positive numbers). In some embodiments, the ASOs are complementary to a targeted portion of the PKD2 NIE containing pre-mRNA that is within the region of about +1 to about +40,000 relative to the 3′ splice site of the intron preceding the included exon. In some aspects, the ASOs are complementary to a targeted portion that is within the region about +1 to about +40,000, about +1 to about +30,000, about +1 to about +20,000, about +1 to about +15,000, about +1 to about +10,000, about +1 to about +5,000, about +1 to about +4,000, about +1 to about +3,000, about +1 to about +2,000, about +1 to about +1,000, about +1 to about +500, about +1 to about +490, about +1 to about +480, about +1 to about +470, about +1 to about +460, about +1 to about +450, about +1 to about +440, about +1 to about +430, about +1 to about +420, about +1 to about +410, about +1 to about +400, about +1 to about +390, about +1 to about +380, about +1 to about +370, about +1 to about +360, about +1 to about +350, about +1 to about +340, about +1 to about +330, about +1 to about +320, about +1 to about +310, about +1 to about +300, about +1 to about +290, about +1 to about +280, about +1 to about +270, about +1 to about +260, about +1 to about +250, about +1 to about +240, about +1 to about +230, about +1 to about +220, about +1 to about +210, about +1 to about +200, about +1 to about +190, about +1 to about +180, about +1 to about +170, about +1 to about +160, about +1 to about +150, about +1 to about +140, about +1 to about +130, about +1 to about +120, about +1 to about +110, about +1 to about +100, about +1 to about +90, about +1 to about +80, about +1 to about +70, about +1 to about +60, about +1 to about +50, about +1 to about +40, about +1 to about +30, or about +1 to about +20, or about +1 to about +10 relative to 3′ splice site of the intron preceding the included exon.

In some embodiments, the targeted portion of the PKD2 pre-mRNA is within the region −4e relative to the 5′ end of the NSE to +2e relative to the 3′ end of the NSE.

In some embodiments, the ASOs are complementary to a targeted region of a PKD2 NIE containing pre-mRNA that is upstream (in the 5′ direction) of the 3′ splice site of the intron preceding the included exon in a PKD2 NIE containing pre-mRNA (e.g., in the direction designated by positive numbers). In some embodiments, the ASOs are complementary to a targeted portion of the PKD2 NIE containing pre-mRNA that is within the region of about +1 to about +40,000 relative to the 3′ splice site of the intron preceding the included exon. In some aspects, the ASOs are complementary to a targeted portion that is within the region about +1 to about +40,000, about +1 to about +30,000, about +1 to about +20,000, about +1 to about +15,000, about +1 to about +10,000, about +1 to about +5,000, about +1 to about +4,000, about +1 to about +3,000, about +1 to about +2,000, about +1 to about +1,000, about +1 to about +500, about +1 to about +490, about +1 to about +480, about +1 to about +470, about +1 to about +460, about +1 to about +450, about +1 to about +440, about +1 to about +430, about +1 to about +420, about +1 to about +410, about +1 to about +400, about +1 to about +390, about +1 to about +380, about +1 to about +370, about +1 to about +360, about +1 to about +350, about +1 to about +340, about +1 to about +330, about +1 to about +320, about +1 to about +310, about +1 to about +300, about +1 to about +290, about +1 to about +280, about +1 to about +270, about +1 to about +260, about +1 to about +250, about +1 to about +240, about +1 to about +230, about +1 to about +220, about +1 to about +210, about +1 to about +200, about +1 to about +190, about +1 to about +180, about +1 to about +170, about +1 to about +160, about +1 to about +150, about +1 to about +140, about +1 to about +130, about +1 to about +120, about +1 to about +110, about +1 to about +100, about +1 to about +90, about +1 to about +80, about +1 to about +70, about +1 to about +60, about +1 to about +50, about +1 to about +40, about +1 to about +30, or about +1 to about +20, or about +1 to about +10 relative to 3′ splice site of the intron preceding the included exon.

In some embodiments, the targeted portion of the PKD2 NIE containing pre-mRNA is within the region +100 relative to the 5′ splice site of the intron following the included exon to −100 relative to the 3′ splice site of the intron preceding the included exon. In some embodiments, the targeted portion of the PKD2 NIE containing pre-mRNA is within the NIE. In some embodiments, the target portion of the PKD2 NIE containing pre-mRNA comprises a NIE and intron boundary.

The ASOs may be of any length suitable for specific binding and effective enhancement of splicing. In some embodiments, the ASOs consist of 8 to 50 nucleobases. For example, the ASO may be 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50 nucleobases in length. In some embodiments, the ASOs consist of more than 50 nucleobases. In some embodiments, the ASO is from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, 12 to 15 nucleobases, 13 to 50 nucleobases, 13 to 40 nucleobases, 13 to 35 nucleobases, 13 to 30 nucleobases, 13 to 25 nucleobases, 13 to 20 nucleobases, 14 to 50 nucleobases, 14 to 40 nucleobases, 14 to 35 nucleobases, 14 to 30 nucleobases, 14 to 25 nucleobases, 14 to 20 nucleobases, 15 to 50 nucleobases, 15 to 40 nucleobases, 15 to 35 nucleobases, 15 to 30 nucleobases, 15 to 25 nucleobases, 15 to 20 nucleobases, 20 to 50 nucleobases, 20 to 40 nucleobases, 20 to 35 nucleobases, 20 to 30 nucleobases, 20 to 25 nucleobases, 25 to 50 nucleobases, 25 to 40 nucleobases, 25 to 35 nucleobases, or 25 to 30 nucleobases in length. In some embodiments, the ASOs are 18 nucleotides in length. In some embodiments, the ASOs are 15 nucleotides in length. In some embodiments, the ASOs are 25 nucleotides in length.

In some embodiments, two or more ASOs with different chemistries but complementary to the same targeted portion of the NIE containing pre-mRNA are used. In some embodiments, two or more ASOs that are complementary to different targeted portions of the NIE containing pre-mRNA are used.

In some embodiments, the antisense oligonucleotides of the disclosure are chemically linked to one or more moieties or conjugates, e.g., a targeting moiety or other conjugate that enhances the activity or cellular uptake of the oligonucleotide. Such moieties include, but are not limited to, a lipid moiety, e.g., as a cholesterol moiety, a cholesteryl moiety, an aliphatic chain, e.g., dodecandiol or undecyl residues, a polyamine, or a polyethylene glycol chain, or adamantane acetic acid. Oligonucleotides comprising lipophilic moieties and preparation methods have been described in the published literature. In embodiments, the antisense oligonucleotide is conjugated with a moiety including, but not limited to, an abasic nucleotide, a polyether, a polyamine, a polyamide, a peptides, a carbohydrate, e.g., N-acetylgalactosamine (GalNAc), N-Ac-Glucosamine (GluNAc), or mannose (e.g., mannose-6-phosphate), a lipid, or a polyhydrocarbon compound. Conjugates can be linked to one or more of any nucleotides comprising the antisense oligonucleotide at any of several positions on the sugar, base, or phosphate group, as understood in the art and described in the literature, e.g., using a linker. Linkers can include a bivalent or trivalent branched linker. In embodiments, the conjugate is attached to the 3′ end of the antisense oligonucleotide. Methods of preparing oligonucleotide conjugates are described, e.g., in U.S. Pat. No. 8,450,467, “Carbohydrate conjugates as delivery agents for oligonucleotides,” incorporated by reference herein.

In some embodiments, the nucleic acid to be targeted by an ASO is a PKD2 NIE containing pre-mRNA expressed in a cell, such as a eukaryotic cell. In some embodiments, the term “cell” may refer to a population of cells. In some embodiments, the cell is in a subject. In some embodiments, the cell is isolated from a subject. In some embodiments, the cell is ex vivo. In some embodiments, the cell is a condition or disease-relevant cell or a cell line. In some embodiments, the cell is in vitro (e.g., in cell culture).

In some embodiments, an ASO that targets a pre-mRNA disclosed herein is selected from the group consisting of the sequences listed in Table 4.

### Pharmaceutical Compositions

Pharmaceutical compositions or formulations comprising the agent, e.g., antisense oligonucleotide, of the described compositions and for use in any of the described methods can be prepared according to conventional techniques well known in the pharmaceutical industry and described in the published literature. In embodiments, a pharmaceutical composition or formulation for treating a subject comprises an effective amount of any antisense oligomer as described herein, or a pharmaceutically acceptable salt, solvate, hydrate or ester thereof. The pharmaceutical formulation comprising an antisense oligomer may further comprise a pharmaceutically acceptable excipient, diluent, or carrier.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66: 1-19 (1977), incorporated herein by reference for this purpose. The salts can be prepared in situ during the final isolation and purification of the compounds, or separately by reacting the free base form with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In some embodiments, the compositions are formulated into any of many possible dosage forms such as, but not limited to, tablets, capsules, gel capsules, liquid syrups, soft gels, suppositories, and enemas. In embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In embodiments, a pharmaceutical formulation or composition of the present disclosure includes, but is not limited to, a solution, emulsion, microemulsion, foam or liposome-containing formulation (e.g., cationic or noncationic liposomes).

The pharmaceutical composition or formulation described herein may comprise one or more penetration enhancers, carriers, excipients or other active or inactive ingredients as appropriate and well known to those of skill in the art or described in the published literature. In embodiments, liposomes also include sterically stabilized liposomes, e.g., liposomes comprising one or more specialized lipids. These specialized lipids result in liposomes with enhanced circulation lifetimes. In embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In embodiments, the present disclosure employs a penetration enhancer to affect the efficient delivery of the antisense oligonucleotide, e.g., to aid diffusion across cell membranes and/or enhance the permeability of a lipophilic drug. In some embodiments, the penetration enhancers are a surfactant, fatty acid, bile salt, chelating agent, or non-chelating nonsurfactant.

In some embodiments, the pharmaceutical formulation comprises multiple antisense oligonucleotides. In embodiments, the antisense oligonucleotide is administered in combination with another drug or therapeutic agent.

### Combination Therapies

In some embodiments, provided herein is a composition comprising one or more NSE-modulating agents. In some embodiments, provided herein is a composition comprising two or more NSE-modulating agents. In some embodiments, provided herein is a composition comprising one or more ASO complementary to a targeted region of PKD2 pre-mRNA. In some embodiments, provided herein is a composition comprising two or more ASO complementary to a targeted region of PKD2 pre-mRNA. In some embodiments, provided herein is a composition comprising one or more ASO complementary to a same targeted region of PKD2 pre-mRNA. In some embodiments, provided herein is a composition comprising two or more ASO complementary to a same targeted region of PKD2 pre-mRNA. In some embodiments, provided herein is a composition comprising one or more ASO complementary to different targeted regions of PKD2 pre-mRNA. In some embodiments, provided herein is a composition comprising two or more ASO complementary to different targeted regions of PKD2 pre-mRNA. In some embodiments, provided herein is a composition comprising one or more ASOs of Table 4. In some embodiments, provided herein is a composition comprising two and more ASOs of in Table 4.

In some embodiments, the therapeutics (e.g., ASOs) disclosed in the present disclosure can be used in combination with one or more additional therapeutic agents. In some embodiments, the one or more additional therapeutic agents can comprise a small molecule. For example, the one or more additional therapeutic agents can comprise a small molecule described in WO2016128343A1, WO2017053982A1, WO2016196386A1, WO201428459A1, WO201524876A2, WO2013119916A2, and WO2014209841A2, which are incorporated by reference herein in their entirety. In some embodiments, the one or more additional therapeutic agents can comprise tolvaptan (Jynarque®, Samsca). In some embodiments, the one or more additional therapeutic agents comprise an ASO that can be used to correct intron retention.

### Treatment of Subjects

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In embodiments, the individual is a human. In embodiments, the individual is a fetus, an embryo, or a child. In other embodiments, the individual may be another eukaryotic organism, such as a plant. In some embodiments, the compositions provided herein are administered to a cell ex vivo.

In some embodiments, the compositions provided herein are administered to an individual as a method of treating a disease or disorder. In some embodiments, the individual has a genetic disease, such as any of the diseases described herein. In some embodiments, the individual is at risk of having a disease, such as any of the diseases described herein. In some embodiments, the individual is at increased risk of having a disease or disorder caused by insufficient amount of a protein or insufficient activity of a protein. If an individual is “at an increased risk” of having a disease or disorder caused insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In embodiments, a fetus is treated in utero, e.g., by administering the ASO composition to the fetus directly or indirectly (e.g., via the mother).

Suitable routes for administration of ASOs of the present disclosure may vary depending on cell type to which delivery of the ASOs is desired. The ASOs of the present disclosure may be administered to patients parenterally, for example, by intrathecal injection, intracerebroventricular injection, intraperitoneal injection, intramuscular injection, subcutaneous injection, or intravenous injection.

In embodiments, the antisense oligonucleotide is administered with one or more agents capable of promoting penetration of the subject antisense oligonucleotide across the blood-brain barrier by any method known in the art. For example, delivery of agents by administration of an adenovirus vector to motor neurons in muscle tissue is described in U.S. Pat. No. 6,632,427, “Adenoviral-vector-mediated gene transfer into medullary motor neurons,” incorporated herein by reference. Delivery of vectors directly to the brain, e.g., the striatum, the thalamus, the hippocampus, or the substantia nigra, is described, e.g., in U.S. Pat. No. 6,756,523, “Adenovirus vectors for the transfer of foreign genes into cells of the central nervous system particularly in brain,” incorporated herein by reference.

In some embodiments, the antisense oligonucleotides are linked or conjugated with agents that provide desirable pharmaceutical or pharmacodynamic properties. In embodiments, the antisense oligonucleotide is coupled to a substance, known in the art to promote penetration or transport across the blood-brain barrier, e.g., an antibody to the transferrin receptor. In embodiments, the antisense oligonucleotide is linked with a viral vector, e.g., to render the antisense compound more effective or increase transport across the blood-brain barrier. In embodiments, osmotic blood brain barrier disruption is assisted by infusion of sugars, e.g., meso erythritol, xylitol, D(+) galactose, D(+) lactose, D(+) xylose, dulcitol, myo-inositol, L(−) fructose, D(−) mannitol, D(+) glucose, D(+) arabinose, D(−) arabinose, cellobiose, D(+) maltose, D(+) raffinose, L(+) rhamnose, D(+) melibiose, D(−) ribose, adonitol, D(+) arabitol, L(−) arabitol, D(+) fucose, L(−) fucose, D(−) lyxose, L(+) lyxose, and L(−) lyxose, or amino acids, e.g., glutamine, lysine, arginine, asparagine, aspartic acid, cysteine, glutamic acid, glycine, histidine, leucine, methionine, phenylalanine, proline, serine, threonine, tyrosine, valine, and taurine. Methods and materials for enhancing blood brain barrier penetration are described, e.g., in U.S. Pat. No. 9,193,969, “Compositions and methods for selective delivery of oligonucleotide molecules to specific neuron types,” U.S. Pat. No. 4,866,042, “Method for the delivery of genetic material across the blood brain barrier,” U.S. Pat. No. 6,294,520, “Material for passage through the blood-brain barrier,” and U.S. Pat. No. 6,936,589, “Parenteral delivery systems,” each incorporated herein by reference.

In some embodiments, an ASO of the disclosure is coupled to a dopamine reuptake inhibitor (DRI), a selective serotonin reuptake inhibitor (SSRI), a noradrenaline reuptake inhibitor (NRI), a norepinephrine-dopamine reuptake inhibitor (NDRI), and a serotonin-norepinephrine-dopamine reuptake inhibitor (SNDRI), using methods described in, e.g., U.S. Pat. No. 9,193,969, incorporated herein by reference.

In some embodiments, subjects treated using the methods and compositions are evaluated for improvement in condition using any methods known and described in the art.

In some cases, a therapeutic agent comprises a modified snRNA, such as a modified human snRNA. In some cases, a therapeutic agent comprises a vector, such as a viral vector, that encodes a modified snRNA. In some embodiments, the modified snRNA is a modified U1 snRNA (see, e.g., Alanis et al., Human Molecular Genetics, 2012, Vol. 21, No. 11 2389-2398). In some embodiments, the modified snRNA is a modified U7 snRNA (see, e.g., Gadgil et al., J Gene Med. 2021; 23:e3321). Modified U7 snRNAs can be made by any method known in the art including the methods described in Meyer, K.; Schtimperli, Daniel (2012), Antisense Derivatives of U7 Small Nuclear RNA as Modulators of Pre-mRNA Splicing. In: Stamm, Stefan; Smith, Christopher W. J.; Lührmann, Reinhard (eds.) Alternative pre-mRNA Splicing: Theory and Protocols (pp. 481-494), Chichester: John Wiley & Sons 10.1002/9783527636778.ch45, incorporated by reference herein in its entirety. In some embodiments, a modified U7 (smOPT) does not compete with WT U7 (Stefanovic et al., 1995).

In some embodiments, the modified snRNA comprises an smOPT modification. For example, the modified snRNA can comprise a sequence AAUUUUUGGAG (SEQ ID NO: 229). For example, the sequence AAUUUUUGGAG (SEQ ID NO: 229) can replace a sequence AAUUUGUCUAG (SEQ ID NO: 230) in a wild-type U7 snRNA to generate the modified U& snRNA (smOPT). In some embodiments, a smOPT modification of a U7 snRNA renders the particle functionally inactive in histone pre-mRNA processing (Stefanovic et al., 1995). In some embodiments, a modified U7 (smOPT) is expressed stably in the nucleus and at higher levels than WT U7 (Stefanovic et al., 1995). In some embodiments, the snRNA comprises a U1 snRNP-targeted sequence. In some embodiments, the snRNA comprises a U7 snRNP-targeted sequence. In some embodiments, the snRNA comprises a modified U7 snRNP-targeted sequence and wherein the modified U7 snRNP-targeted sequence comprises smOPT. In some embodiments, the modified snRNA has been modified to comprise a single-stranded nucleotide sequence that hybridizes to a PKD2 pre-mRNA. In some embodiments, the modified snRNA has been modified to comprise a single-stranded nucleotide sequence that hybridizes to a PKD2 mRNA.

In some embodiments, the modified snRNA has been modified to comprise a single-stranded nucleotide sequence that hybridizes to a target region of a PKD2 pre-mRNA or a processed PKD2 mRNA, such as a target region of a PKD2 pre-mRNA that modulates exclusion of an NMD exon, a target region of a PKD2 pre-mRNA that modulates splicing at an alternative 5′ ss, or a target region of a processed PKD2 mRNA that modulates translation of a PKD2 mRNA, such as a 5′ UTR target region. In some embodiments, the modified snRNA has been modified to comprise a single-stranded nucleotide sequence that comprises one or two or more sequences of the ASOs disclosed herein. In some embodiments, the modified snRNA has been modified to comprise a single-stranded nucleotide sequence that hybridizes to sequence of a PKD2 pre-mRNA with a mutation, such as a PKD2 NMD exon-containing pre-mRNA with a mutation. In some embodiments, the modified snRNA has been modified to comprise a single-stranded nucleotide sequence that comprises two or more sequences that hybridize to two or more target regions of a PKD2 NMD exon-containing pre-mRNA. For example, a modified snRNA can be modified to comprise a single-stranded nucleotide sequence that hybridizes to at least 8 contiguous nucleic acids of a PKD2 NMD exon-containing pre-mRNA. In some embodiments, the modified snRNA has been modified to comprise a single-stranded nucleotide sequence that hybridizes to any of the target regions of a PKD2 NMD exon-containing pre-mRNA disclosed herein. In some embodiments, the modified snRNA has been modified to comprise a single-stranded nucleotide sequence that comprises two or more sequences that hybridize to two or more target regions of a PKD2 NMD exon-containing pre-mRNA. For example, a modified snRNA can be modified to comprise a single-stranded nucleotide sequence that hybridizes to one or two or more sequences of an intron containing an NMD exon (e.g., exon 2× of PKD2 (e.g., exon (GRCh38/hg38: chr4:88031085 88031140) of PKD2) of the pre-mRNA transcript or to a NMD exon-activating regulatory sequence in the same intron. For example, a modified snRNA can be modified to comprise a single-stranded nucleotide sequence that hybridizes to a region within an NMD exon or upstream or downstream of an NMD exon (e.g., exon 2× of PKD2 (e.g., exon (GRCh38/hg38: chr4:88031085 88031140) of PKD2). In some embodiments, the modified snRNA has a 5′ region that has been modified to comprise a single-stranded nucleotide sequence that hybridizes to a PKD2 NMD exon-containing pre-mRNA. In some embodiments, the modified snRNA has a 3′ region that has been modified to comprise a single-stranded nucleotide sequence that hybridizes to a PKD2 NMD exon-containing pre-mRNA.

In some embodiments, the modified snRNA has been modified to comprise a single-stranded nucleotide sequence that hybridizes to a target region of a PKD2 pre-mRNA that modulates exclusion of an NMD exon. For example, a modified snRNA can be modified to comprise a single-stranded nucleotide sequence that hybridizes to a region that overlaps with an NMD exon and an intron upstream of the NMD exon (e.g., exon 2× of PKD2 (e.g., exon (GRCh38/hg38: chr4:88031085 88031140) of PKD2). For example, a modified snRNA can be modified to comprise a single-stranded nucleotide sequence that hybridizes to a region that overlaps with an NMD exon and an intron downstream of the NMD exon (e.g., exon 2× of PKD2 (e.g., exon (GRCh38/hg38: chr4:88031085 88031140) of PKD2). For example, a modified snRNA can be modified to comprise a single-stranded nucleotide sequence that is complementary to an intron sequence that is downstream of an NMD exon (e.g., exon 2× of PKD2 (e.g., exon (GRCh38/hg38: chr4:88031085 88031140) of PKD2). For example, a modified snRNA can be modified to comprise a single-stranded nucleotide sequence that is complementary to a 3′ splice site of an intron sequence that is downstream of an NMD exon (e.g., exon 2× of PKD2 (e.g., exon (GRCh38/hg38: chr4:88031085 88031140) of PKD2). For example, a modified snRNA can be modified to comprise a single-stranded nucleotide sequence that is complementary to a 5′ splice site of an intron sequence that is downstream of an NMD exon (e.g., exon 2× of PKD2 (e.g., exon (GRCh38/hg38: chr4:88031085 88031140) of PKD2). For example, a modified snRNA can be modified to comprise a single-stranded nucleotide sequence that is complementary to an intron sequence that is upstream of an NMD exon (e.g., exon 2× of PKD2 (e.g., exon (GRCh38/hg38: chr4:88031085 88031140) of PKD2). For example, a modified snRNA can be modified to comprise a single-stranded nucleotide sequence that is complementary to a splice site of an intron sequence that is upstream of an NMD exon (e.g., exon 2× of PKD2 (e.g., exon (GRCh38/hg38: chr4:88031085 88031140) of PKD2). For example, a modified snRNA can be modified to comprise a single-stranded nucleotide sequence that is complementary to a 3′ splice site of an intron sequence that is upstream of an NMD exon (e.g., exon 2× of PKD2 (e.g., exon (GRCh38/hg38: chr4:88031085 88031140) of PKD2). For example, a modified snRNA can be modified to comprise a single-stranded nucleotide sequence that is complementary to a 5′ splice site of an intron sequence that is upstream of an NMD exon (e.g., exon 2× of PKD2 (e.g., exon (GRCh38/hg38: chr4:88031085 88031140) of PKD2).

In some embodiments, the modified snRNA has been modified to comprise a single-stranded nucleotide sequence that hybridizes to a target region of a PKD2 pre-mRNA that modulates splicing at an alternative 5′ ss. For example, a modified snRNA can be modified to comprise a single-stranded nucleotide sequence that hybridizes to a region that overlaps with an alternative 5′ ss of an intron of a PKD2 pre-mRNA (e.g., an alternative 5′ ss of intron 3 of PKD2 (e.g., GRCh38/hg38: chr4 88036480) of PKD2). For example, a modified snRNA can be modified to comprise a single-stranded nucleotide sequence that hybridizes to a region that overlaps with an alternative 5′ ss of an intron of a PKD2 pre-mRNA and an NMD exon upstream of the alternative 5′ ss (e.g., an alternative 5′ ss of intron 3 of PKD2 (e.g., GRCh38/hg38: chr4 88036480) of PKD2 and an NMD exon upstream of the alternative 5′ ss (e.g., exon 2)). For example, a modified snRNA can be modified to comprise a single-stranded nucleotide sequence that is complementary to an intronic sequence that is downstream of an alternative 5′ ss of an intron of a PKD2 pre-mRNA (e.g., an intronic sequence that is downstream of an alternative 5′ ss of intron 3 of PKD2 (e.g., GRCh38/hg38: chr4 88036480) of PKD2). For example, a modified snRNA can be modified to comprise a single-stranded nucleotide sequence that is complementary to a sequence of an NMD exon (e.g., an alternative exon) that is upstream of an alternative 5′ ss of an intron of a PKD2 pre-mRNA (e.g., a sequence of an NMD exon (e.g., an alternative exon) that is upstream of an alternative 5′ ss of intron 3 of PKD2 (e.g., GRCh38/hg38: chr4 88036480) of PKD2). For example, a modified snRNA can be modified to comprise a single-stranded nucleotide sequence that is complementary to an alternative 5′ splice site of an intron that is downstream of an NMD exon (e.g., an alternative exon) (e.g., an alternative 5′ splice site of intron 3 (e.g., GRCh38/hg38: chr4 88036480) of PKD2 that is downstream of an NMD exon (e.g., an alternative exon)).

In some embodiments, the modified snRNA has been modified to comprise a single-stranded nucleotide sequence that hybridizes to a target region of a PKD2 mRNA that modulates translation of a processed PKD2 mRNA, such as a 5′ UTR target region. For example, a modified snRNA can be modified to comprise a single-stranded nucleotide sequence that hybridizes to a 5′ UTR sequence of a processed PKD2 mRNA. For example, a modified snRNA can be modified to comprise a single-stranded nucleotide sequence that hybridizes to an upstream open reading frame start codon of a processed PKD2 mRNA. For example, a modified snRNA can be modified to comprise a single-stranded nucleotide sequence that hybridizes to a sequence upstream of an upstream open reading frame start codon of a processed PKD2 mRNA. For example, a modified snRNA can be modified to comprise a single-stranded nucleotide sequence that hybridizes to a sequence downstream of an upstream open reading frame start codon of a processed PKD2 mRNA. For example, a modified snRNA can be modified to comprise a single-stranded nucleotide sequence that hybridizes to a sequence upstream of a canonical open reading frame start codon of a processed PKD2 mRNA. For example, a modified snRNA can be modified to comprise a single-stranded nucleotide sequence that hybridizes to a sequence downstream of a first upstream open reading frame start codon of a processed PKD2 mRNA and upstream of a second upstream open reading frame start codon of a processed PKD2 mRNA. For example, a modified snRNA can be modified to comprise a single-stranded nucleotide sequence that hybridizes to a sequence upstream of a first upstream open reading frame start codon of a processed PKD2 mRNA and upstream of a second upstream open reading frame start codon of a processed PKD2 mRNA. For example, a modified snRNA can be modified to comprise a single-stranded nucleotide sequence that hybridizes to a sequence upstream of a first upstream open reading frame start codon of a processed PKD2 mRNA, upstream of a second upstream open reading frame start codon of a processed PKD2 mRNA and upstream of a canonical open reading frame start codon of a processed PKD2 mRNA.

Methods of Identifying Additional ASOs that Induce Exon Skipping

Also within the scope of the present disclosure are methods for identifying or determining ASOs that induce exon skipping of a PKD2 NIE containing pre-mRNA. For example, a method can comprise identifying or determining ASOs that induce NIE skipping of a PKD2 NIE containing pre-mRNA. ASOs that specifically hybridize to different nucleotides within the target region of the pre-mRNA may be screened to identify or determine ASOs that improve the rate and/or extent of splicing of the target intron. In some embodiments, the ASO may block or interfere with the binding site(s) of a splicing repressor(s)/silencer. Any method known in the art may be used to identify (determine) an ASO that when hybridized to the target region of the exon results in the desired effect (e.g., NIE skipping, protein or functional RNA production). These methods also can be used for identifying ASOs that induce exon skipping of the included exon by binding to a targeted region in an intron flanking the included exon, or in a non-included exon. An example of a method that may be used is provided below.

A round of screening, referred to as an ASO “walk” may be performed using ASOs that have been designed to hybridize to a target region of a pre-mRNA. For example, the ASOs used in the ASO walk can be tiled every 5 nucleotides from approximately 100 nucleotides upstream of the 3′ splice site of the intron preceding the included exon (e.g., a portion of sequence of the intron located upstream of the target/included exon) to approximately 100 nucleotides downstream of the 3′ splice site of the intron preceding the target/included exon and/or from approximately 100 nucleotides upstream of the 5′ splice site of the intron following the included exon to approximately 100 nucleotides downstream of the 5′ splice site of the intron following the target/included exon (e.g., a portion of sequence of the intron located downstream of the target/included exon). For example, a first ASO of 15 nucleotides in length may be designed to specifically hybridize to nucleotides +6 to +20 relative to the 3′ splice site of the intron preceding of the target/included exon. A second ASO may be designed to specifically hybridize to nucleotides +11 to +25 relative to the 3′ splice site of the intron preceding the target/included exon. ASOs are designed as such spanning the target region of the pre-mRNA. In embodiments, the ASOs can be tiled more closely, e.g., every 1, 2, 3, or 4 nucleotides. Further, the ASOs can be tiled from 100 nucleotides downstream of the 5′ splice site, to 100 nucleotides upstream of the 3′ splice site. In some embodiments, the ASOs can be tiled from about 1,160 nucleotides upstream of the 3′ splice site, to about 500 nucleotides downstream of the 5′ splice site. In some embodiments, the ASOs can be tiled from about 500 nucleotides upstream of the 3′ splice site, to about 1,920 nucleotides downstream of the 3′ splice site.

One or more ASOs, or a control ASO (an ASO with a scrambled sequence, sequence that is not expected to hybridize to the target region) are delivered, for example by transfection, into a disease-relevant cell line that expresses the target pre-mRNA (e.g., a NIE containing pre-mRNA described herein). The exon skipping effects of each of the ASOs may be assessed by any method known in the art, for example by reverse transcriptase (RT)-PCR using primers that span the splice junction, as described in Example 4. A reduction or absence of a longer RT-PCR product produced using the primers spanning the region containing the included exon (e.g., including the flanking exons of the NIE) in ASO-treated cells as compared to in control ASO-treated cells indicates that splicing of the target NIE has been enhanced. In some embodiments, the exon skipping efficiency (or the splicing efficiency to splice the intron containing the NIE), the ratio of spliced to unspliced pre-mRNA, the rate of splicing, or the extent of splicing may be improved using the ASOs described herein. The amount of protein or functional RNA that is encoded by the target pre-mRNA can also be assessed to determine whether each ASO achieved the desired effect (e.g., enhanced functional protein production). Any method known in the art for assessing and/or quantifying protein production, such as Western blotting, flow cytometry, immunofluorescence microscopy, and ELISA, can be used.

A second round of screening, referred to as an ASO “micro-walk” may be performed using ASOs that have been designed to hybridize to a target region of a pre-mRNA. The ASOs used in the ASO micro-walk are tiled every 1 nucleotide to further refine the nucleotide acid sequence of the pre-mRNA that when hybridized with an ASO results in exon skipping (or enhanced splicing of NIE).

Regions defined by ASOs that promote splicing of the target intron are explored in greater detail by means of an ASO “micro-walk”, involving ASOs spaced in 1-nt steps, as well as longer ASOs, typically 18-25 nt.

As described for the ASO walk above, the ASO micro-walk is performed by delivering one or more ASOs, or a control ASO (an ASO with a scrambled sequence, sequence that is not expected to hybridize to the target region), for example by transfection, into a disease-relevant cell line that expresses the target pre-mRNA. The splicing-inducing effects of each of the ASOs may be assessed by any method known in the art, for example by reverse transcriptase (RT)-PCR using primers that span the NIE, as described herein (see, e.g., Example 4). A reduction or absence of a longer RT-PCR product produced using the primers spanning the NIE in ASO-treated cells as compared to in control ASO-treated cells indicates that exon skipping (or splicing of the target intron containing an NIE) has been enhanced. In some embodiments, the exon skipping efficiency (or the splicing efficiency to splice the intron containing the NIE), the ratio of spliced to unspliced pre-mRNA, the rate of splicing, or the extent of splicing may be improved using the ASOs described herein. The amount of protein or functional RNA that is encoded by the target pre-mRNA can also be assessed to determine whether each ASO achieved the desired effect (e.g., enhanced functional protein production). Any method known in the art for assessing and/or quantifying protein production, such as Western blotting, flow cytometry, immunofluorescence microscopy, and ELISA, can be used.

ASOs that when hybridized to a region of a pre-mRNA result in exon skipping (or enhanced splicing of the intron containing a NIE) and increased protein production may be tested in vivo using animal models, for example transgenic mouse models in which the full-length human gene has been knocked-in or in humanized mouse models of disease. Suitable routes for administration of ASOs may vary depending on the disease and/or the cell types to which delivery of the ASOs is desired. ASOs may be administered, for example, by intrathecal injection, intracerebroventricular injection, intraperitoneal injection, intramuscular injection, subcutaneous injection, or intravenous injection. Following administration, the cells, tissues, and/or organs of the model animals may be assessed to determine the effect of the ASO treatment by for example evaluating splicing (e.g., efficiency, rate, extent) and protein production by methods known in the art and described herein. The animal models may also be any phenotypic or behavioral indication of the disease or disease severity.

Also within the scope of the present disclosure is a method to identify or validate an NMD-inducing exon in the presence of an NMD inhibitor, for example, cycloheximide. An exemplary method is provided in Example 2.

Exemplary genes are summarized in Table 1. The sequence for each intron is summarized in Table 2.

TABLE 1
List of exemplary target gene sequences

| Gene | Gene | | | |
|---|---|---|---|---|
| Symbol | ID No. | Disease | OMIM | Genetics |
| PKD2 | 5311 | polycystic kidney disease | 613095 | Haploinsuf- |
| | | with or without polycystic | | ficient, Loss- |
| | | liver disease, autosomal | | of-function |
| | | dominant polycystic kidney | | |
| | | disease, or intracranial | | |
| | | aneurysm | | |

TABLE 2
PKD2 sequences
PKD2

| | |
|---|---|
| genomic | AGGCGGCGGCGGGCGCCGGGAAGAAAGGAACATGGCTCCTGAGGCGCACAG |
| sequence | CGCCGAGCGCGGCGCCGCGCACCCGCGCGCCGGACGCCAGTGACCGCGATG |
| | GTGAACTCCAGTCGCGTGCAGCCTCAGCAGCCCGGGGACGCCAAGCGGCCG |
| | CCCGCGCCCCGCGCGCCGGACCCGGGCCGGCTGATGGCTGGCTGCGCGGCCG |
| | TGGGCGCCAGCCTCGCCGCCCCGGGCGGCCTCTGCGAGCAGCGGGGCCTGG |
| | AGATCGAGATGCAGCGCATCCGGCAGGCGGCCGCGCGGGACCCCCCGGCCG |
| | GAGCCGCGGCCTCCCCTTCTCCTCCGCTCTCGTCGTGCTCCCGGCAGGCGTGG |
| | AGCCGCGATAACCCCGGCTTCGAGGCCGAGGAGGAGGAGGAGGAGGTGGAA |
| | GGGGAAGAAGGCGGAATGGTGGTGGAGATGGACGTAGAGTGGCGCCCGGGC |
| | AGCCGGAGGTCGGCCGCCTCCTCGGCCGTGAGCTCCGTGGGCGCGCGGAGCC |
| | GGGGGCTTGGGGGCTACCACGGCGCGGGCCACCCGAGCGGGAGGCGGCGCC |
| | GGCGAGAGGACCAGGGCCCGCCGTGCCCCAGCCCAGTCGGCGGCGGGGACC |
| | CGCTGCATCGCCACCTCCCCCTGGAAGGGCAGCCGCCCCGAGTGGCCTGGGC |
| | GGAGAGGCTGGTTCGCGGGCTGCGAGGTAAGAGCGCGCGACCCGCAGCGGC |
| | AGATGCACGAACCAGAACGGCCGGCGCCGGCCGGGGCCATCGCCCGCTGCG |
| | GCAGCTCCCCGGGCTCCATCTCGCATCCCCTCTGCGTTCCGCCTCCCTTGGAA |
| | GCGCATTCCCCACCTCCGCTAGTGCTGCCCTATTTCCGGTACCCAGCGCGGA |
| | ATTCCACTGCTCTTTTGTTGGTGCATATTTATTGGATACCTCCTTCTTCAGGAT |
| | ATGTCACCATAGTCTTTTTTACTGAAAATTAGTGAAAGCCTAATTAGAGTGA |
| | AAGAGTACATCTGGGTTTTGTTTTTTTTTTTCTTGTAGAGGAAAAAATGAACA |
| | TTACTTGTGTAACTGATGGTAGTTGCAACTGCATATTTGCCAATGTCACAAAA |
| | TCTAAAGGAAAATGTTATAGTCACCCGTGGTTTCCTTCTTGCCTGGACACTCC |
| | ATTGTCCCGGGCTGAAAAGGGTAGCAGTACAGTGCATATAATGTCAAGTTGT |
| | GGGAGGAGTGTGGCAGATTGTCATTGGTGCATTTTTTTGGTGATGTGTGTGGT |
| | TGTTTTGAGGAGTGGGAGCTGTTAAGAACACCACAAATAGAATAAAATAATA |
| | TCGTGAAGTTATTTGGCCGTTTCTAATTCTAGACATTTTTCTAAAAACAGTTG |
| | CAAAGGAAAGATTACATTGTTTTAAAAAAATTTGAAGTATGTTTTTAAATAA |
| | CTAAATTAATGTTCTTTGAAATTCCACCAAAATGATGAAGTCACCAGATAGC |
| | AGCTGATAAATGTGTCTGAGCCCAGTGCGCCCAGCTCTACAAAAGGCAGAAG |
| | AGGAATTTTCAAATTTGCCAGTGCCCAGTAAGAGGACATGAATTTTCTAGTA |
| | CCAGAGGAAATTCTCTTTTTACAAATTTTGTCAGAGGTATCCTTGGGAAAGTA |
| | TTTCATTTGCTTTTACCCTCCAAATTATTTTAATCTATATTTTTAAGAGTTTCC |
| | ATTCTCAGTTGAGTTTTTCTTGTTCTTCTCCTTCTGTCAGTTTTGAAAGTCTTG |
| | CACAAAAACAATCCAGGTGTTGTTACAGCAGTGTGATTAAAACCAGGTCAGG |
| | CCTACACTGAAATCCCAGTTCCACCACTCATTAGCTGTACGACCTTGAGCAA |
| | GTTACTTATCCTCTCTGATACCCAATTTTTGGATTTTTTTTTTTTTTAAAGAGA |
| | TGATCATAACGCTTAGGACTTGACTCTTTGGGAGAATTAAGTGAGTTAAGAC |
| | ATAAAATGTGCAGCATGTATTTGTCATGTTATTAGCGCTTCAGAAATATAAAT |
| | GTAAACATTTTGGTACTTCGTTTATGGAGATTCTTATACTAGTTAATTTTATTA |
| | AAAGCATGATGGGGAACAGAAGATCCTTTTCGGATAACCTGTGTGAGTAAAT |
| | TAATAAAACACTAACACTTTTTAAGATTCAAAACTGGATTAATTATGTTATTT |
| | TACACCATTTAAAATGTGCATTTAAAAAATATTCACTGAAGTGAGAGAGAAG |
| | TTCCTTTTAAGTTAATAAATAATGGTAAGTTGCATGATCCTTTTAACTCAGTT |
| | TAAGCATTTGATAACACCCCTAACCTTGTTTGAATAAACTCAATAACAGAAT |
| | ATAGAGAAATAAAATATATATTTCATATGAGTATGTATGTAAATTTATTTCTT |
| | TTAAAGGAAAATTTCAGGAAACAAAATGAATAAGCTCATAGTCATAAAACCT |
| | CTAGCCAAAGTGTGTCAATGGTCTGATTTGTAGGAGAGCAGACTCAGGAATC |
| | GGGAATTTTTTTTTTTTTTTTAAGAGTCGGGGTTTCACTCTGTCACTGAGGCTG |
| | GAGTGCGGGGAAGCAAACGTAGCTCACTGCGGCCTCAAACTCCTGGGCTCAA |
| | GCTGCCCTCCCACCTCAGCTTCCCAAGTAGTTGGGATTACAGGTGCAAGCTG |
| | CTGCTCCTAGCTGAAAAGTTTTAATTATATAAAATGTTTAAATAAATTGTTAT |
| | TCCTTTCTTTTATGAAGAAATATAATTGATATTCTTGTCACTTATTAAATAAG |
| | CAACATTTTAAAATGTTTAGCACCTACTGTGAAGATAGCACTGTGCTAGCTG |
| | CTATGAATAAACTAGAATAGCATATCACTGTTTTCCTATGGACAAAAACCCA |
| | GAAATGTAAAATAAATAGCCATGAATGGGTTGAGATTCCCCCTGCCCCCTTT |
| | GGATACTGATGACAGAAATCTTATATGCACTATGTAAGCAGTGCCCTAGGAT |
| | CAGAACAGAAAGTAACTTGCAATTTTTAAAGGTGGGAGAAATGACTGAAGTT |
| | TGGAGTGGTCAGGAGTTTCCCTGGAAGTCTCTTGCCTTTGTGACTTTGTATAG |
| | CTCCCTCAAACTACCTGAGCCTGAGCTCCTCATTTATAAAATGGAAGAATTG |
| | AACCTAGCTTCCAAGGTCCTTTCTAGCTTCACCATGCTCTGGTCTGTTGTTTA |
| | ATGACATGAATCCAAGATGGACAACAAATGGTCGATTTTGCTCCTTCTACAG |
| | TAAATAGATCTATCTTCTTAGCAGAAGTAAAATAGTAAAGAAAGAAGACATG |
| | TTTGAGGCCTGTTGAGGCCTGTTTGCTGTATGCATTGTATCTAATCAAGGAAT |
| | TGAGAATGTAGCCCTAAATATTAGGAAGGAGTTGAAAGTTTCTGAGCAGGAA |
| | CAGAACATGCTGAAAGGAAATTGTGTCAGCAGCATGGTGTACAGGAGAGCT |
| | TGGCAGAGAGAAAGGAGGCAAGAAGACCTGTGGGAGGCAGCCAGTAAGGA |
| | GGTGAAGAGGGCCTGGACCAAAGGAAGCCAAGGATGACCGAAAGATTTAAA |
| | GATGAAGCCCAGATTTAAAAGTATCCTCAAGTATTTGTTTATTCTTTTCTTAG |
| | CAAATTCTTTTTAGTACAAAGATAAAATATGGCACTCTGAGTCATAAAATTTT |
| | CTGAATTTCAGAAGTTGAATGTTTTTCTGAATGTATCAACCTGTTAAAGTCAG |
| | TTCCTGTTTGTTATTTTAGGCGTATATTCTTGGGCTTTTTTTTTTTTTTCCTAGA |
| | GAAACTATGAAGTACTAGCTGTGCAAGTCAGGGTGGGCTAAGCTGCTAAAAC |
| | AGATACCTCCCTCCCCTTCAATACCCTGAAGAACAAGAAGTTAATTTTTTGCT |
| | CGTGTAACATTTTAGAGAGGGTATTCCAGGTTGAATACCTGGAATAATGAAT |
| | GAAATAATAAGTCGACTCTTATTTCTCCTAAAATAATTGGATCTTGACATTGA |
| | AATTGGTGTGCTGATTGTATTTAATAAGTGACCTCCAATGCAGTTATTTCATT |
| | TTGCCATACTTTATGTAATTTTTATTTTTTCTGCCTTCCCTTTCGATGTCTAAA |
| | GGGAAGCATAATTAGTTATTGGAAAGGTTATTGAGACTTAAAAAAAGATTTG |
| | AACATGGCTTTGGTTGATGCTACCACACAGAAACTGCAAACATCCAACTCAC |
| | CGCTCTTAGTTGCTGTTCATGATAGTCTATGCACAGTGACTGATGAATTTAGC |
| | CTGTCTTGAGGCTTCAGAACTTAGTCTTATGTCATCACGAGCAGAGCTTTATC |
| | CTAAATTACAGGTTTTCAATGGGGGGGGGGGGGAGGGGCAGTTTTGCCCCTC |
| | AGGGGACATTTGGCAATCTCTGGAGACATTTTTGATTGTCATACCTGGGGGG |
| | ATGCCCCTGGCATCTCCTGGGTTGAGGTTAAGGATGCTGCTAATCTACAATG |
| | CACAGTACAGCCACCCCACACACACACACTACAAAGCATTCTCCCACCCAAA |
| | TTGCCAGGAGTGAGGACTTTGAGAAACCCTGCCCTAGACTGTTAAATTCAAA |
| | AGGAAATAATGGTTTATTGCTCACAGTGTGCCAGGCACTGTGTTAAACTCCTT |
| | ATATTCATAGTTTTGTTTTTATCCTCACAACAACCTGTGAAGAAAGAACTCTC |
| | ATCCGTCACCACTTTACTGTTGAGGGCACTAAGCCTTCTAAAGGTTAAATGA |
| | CTTGCCCAGGGCCGCAACTATTAGATGTTGCAGTCAGGATTTAATTCCAGGC |
| | ACTTTGTGTTCAAAGTGTTTCTCATCCACTGTGCTATATGCCAGTAGTGCCCA |
| | AACCTAACTTTAGCCAGCAATTGTCTGCATCTCTTCAGTTTATGAACATTTAT |
| | TTATTAGGACATGCAGGATAATCATACCAACACAGTCCGTGTATCCAGAATT |
| | CTAATTGATCTAGGGAGTGGGAGAGCCTGCCTCTCTATTTTTTTTAAATTGGT |
| | GTGAAATATACATAACATAAAATTGGCCATTTTAACCATTTTAAGTATATAAT |
| | TGAGTGACATTAGGTAGATTTATTATATTATGTAAATGGACTTAACACAATTT |
| | ATTTCCAGAACTTTTTTATCATCCCAAACAGAAACTCTGTACTCATTAAACAG |
| | TAACTCCCTGTTTCCCCACTCCCCCTTGCCCCAGACGCTAGTAACCTCCATTC |
| | TACTTCCCCTTTCTGTGAGTCTCCCTGTTGTAGCTACCTGATGTAAGTGAAAT |
| | CAGACCATGTTTGATCTTTTGTGTCTGGCTTATTCACTTAGCATAGTATTTTCA |
| | AGGTTTATCCATGCGTGCAAATTCCCTTCTTTTTTATGACGAAATACTATTTA |
| | ATTGTGTGTGTACATGTGCACACACAATGAAATACTATTTCATCATAAAAAG |
| | GAAGTTTGTACACATATATACACACACACCAAATTTTGTTTATCCATTCATCA |
| | GTTCATGGGCATTTGCGGTTTTTTTCCACCTTTCGACTGTTGTGACTTTCCGTT |
| | TATTTTTAATCTGAACTTTACTCCATCACTTCCTCCCTTTCCTTTTTTATTCGCA |
| | CCATAATTTTGAACAAGCAGACTTTGTATTTTCATTACTCTGGGGATTTTTTT |
| | GAGGGAGGCTTGCTACCCTTTGGGGTTGTGGTAAGGTTGTGCCAGTAACAGA |
| | ATTCATGCAGTAAAAAACATTCATGGGACTTTCTTTTGTGATAGGTACTAGG |
| | GATGCAGAGATGAATAATACAAGGTCTCGACCTTCAAGGAGCTCAGGGTTTA |
| | GTAGGGAAAACAGAGGTATAAGTAAGTCATTGCAATGCCAGGTCCCAGATA |
| | TGGGAGGATTCCAGCAAAGAGAGGATAAGGCCAGGCACAGCCGCTCATGCC |
| | TATAGTCCCAACACTTTGGGAAGCTGAGATGGGAGGATCACTTAAGCCCAGC |
| | AGTTCAAGGCTACCCTGGGCAACATAGTGAGTGGCAAAAAATACAAAAATT |
| | AGCCAGGCAGGGTGGCACATGCCTGTGGTCCCAGTTATTTGGGAGTTTGAGG |
| | TGGGAGAATCACCTGAGCCTGGGAGATTGAGGCTGCAGTGAGCTGTGATCAC |
| | GCCACTGCACTCCAGCCTGGGTGACACAGTGAGAGGTGAGAGCCTGTATCTA |
| | AAAAAAAAAAAAAGAGAGGATCAGATTTACCTGGAGAGGTCAGAGAAGGTT |
| | TCCAGAGGGAGTAAAACTTGAGCTTCATTTTGAAGAATGAGAGGGGAATAC |
| | AAGGAGGGAAAAATAATAGGAACAAGACCTGTTCCTGTTACAGCAAACCTG |
| | TAACTCCCACCATGGAATGGGCACGTTTTCCTGTAGACATTTGGAGCCAGTG |
| | TGGGCTTCAAGCCTAGGGGGCTGATATGATCATTTGTCAATCTGAAGATGAC |
| | TCTTGGAACAACGTAGAGGATAGGGTGAGGTGGGTAGGCTGGTGGCTGACA |
| | GACTAGGTCAGGAGAGGGCTGACCAAGAGGAGGAGGGGTGACGAGGGCTG |
| | GAACTAAGGCATATGAGCTGGGATGGAAAGAAGCTAGATAGAAAGGAAACA |
| | AAAACCATCAGAACTGTGGAACCAACTCCATGGGAGGGATAAGAAGAGGGC |
| | CTATTCTAGGGTGAAACCCAAATTTCTGGCATGGTACCATTAACCCAAACAG |
| | AAAGAGGAGATTTCCAGAGAAAGAAAAGCAAATTTTGCGGGAGGTGAGTGT |
| | GAGCTGTCTAGAGAATATGCAGGTAGAACTACATGTTAACTGTAACATATAC |
| | TTGTCTGCACCTCAGGAGATGGCTGATTAAGAATTCAGGATTCAGGCCAGGC |
| | ACGGTGGCACACGCCTTTAATCCCAGCTGCTTAAGAGACTGGGGGGGGAGGA |
| | TTGCTTGAGCCTGGGAATCCGAGATCAGCCTGGGCAACATAGCAAGACACTG |
| | TCTCTAAAAAAAAAAAAAAATTCAAGATTCTGGAGTCAATATTACTTAAGGT |
| | AGCAACTGTCTATTCTATAGAAAATGGACAAATATAGATAAAAAGCCACTTC |
| | CCTTTCTAAAAGTGTCCCTGCAATTCAAGTGAATACTAGGAAGGATTGTGTTC |
| | ATTCTTCTAAACAAGACTCACATGTATCTTAGCACAAAAAGAGGATTCTTTTA |
| | TGATACAAATGCACTGAGAATTTGGTCAGGCTATCACAATGAACTGATAGTT |
| | CAGATGGATTTGAGTCTTTATACCACTCTGGAATCTGGACCAACTGGGCCTCC |
| | TAAGGCCATTTTGCAGATCTGGGCTTGTTTCTGAAGCTACAGACAGGCCTCTT |
| | CCAAGCACTCTAAGTGCTCCACAAATAATATTTCTGTTTCCCAGAACAACCCC |
| | ACAAAAAGGTACTCTTACTCCATTTTTAGATGAGGAAGTGGAGGCTCATGAT |
| | GTCAGGTAAGCTTTCTCAGCTCCCAAGTGGTTAAGCCCTCAGTTTAATGTCAT |
| | TTGACTCCAGAGCCCTATGTTGCACCATGCCTTGATAATAGGCCATATGGGTT |
| | TCATGTATTTCAGATGGGGAAGGTTAGTGTGAGGTGAAAGATACACAATTAA |
| | CCTTTTAACCATGGAACTGAAATATTTACAGATGAAGTGATACAATAGCTGG |
| | AATTAATTCCAAAATAATTGGGGTGGTACTGGTCCATGGCCTGTTAGGAACT |
| | GGGCCGCAGAGCAGGAGGTGAGCAGCAGGCTAGTGAGCATTGCTACCCCCT |
| | ATCATATCAGCAGTGGCATCAGATGGGTGAGGATGTAAATGAACTAGGATTG |
| | GCATTGAGTTGATATTGTTGGATCTAGGTGATAGATTTATCATTATAATATTC |
| | TCTCTTTGTGATATTTCTGATATTTTCTAAAATAAAAAGTTGTAGTTATTTATT |
| | TATTTAGACGGAGTTTCACTCTTTTGCCCAGGTTGGAGTGCAGTGGTGTGATC |
| | TCGGCTCACTGTAGCCTCCGCCTCCCGGGTTCAAGCGATTCTCCTGCCTCAGC |
| | CTCCCGAGTAGCTGGGACTACAGCCTCCCGAGTAGCTCACCACCACACCCAG |
| | CTAACTTTTGTATTTTTAGTAGAGAGGAGGTTTCACCATGTTAGATAGGCTGG |
| | TCTCAAACTCCTGACCTCAGGTGATCCACCCACCTCGGCCTCCCAGAGTGCT |
| | GGGATTACAGGCGTGAGCCAGTGTGCCCAGCCAATAAAAAGTTTTTGAAAGG |
| | ATTTAGATAAAATAGTTGGGGAAATGGCATTTTTGTTCAAAGCCAATTATTTA |
| | TGTTTGGAATATCTTTTGTGCTTGGAGTTCTCCATTACAGAGTTCCCCAGTGT |
| | TCCTATTAATAAGTAACATTGAGCAGAGGAATGCACTGTTTAGATCAGCAGT |
| | CCCCAACCTTTTTGGCACCAGGGACCGGCTTCATGGAAGACAATTTTTCCAC |
| | AGACCTGGGGTAGGAAGTGTTTGGGGATGAAACTGTTCCACCTCAGATCATC |
| | AGGCATTAGTGAGATTCTCATAAGGAGCAGGCAATCTAGATTCTTCACATGC |
| | GCAGTTCACAATAGGGTTCTCACTCCTGTGAGAATCTAATGCCACCCCTGATC |
| | TGACAGGAGGCGGAGCTCGGGCGGTAATGCTCACTTGCCTGCGGCTCACCTC |
| | CTGCTGTGGGGCCCGGTTCCTAACAGGCCATGGACCAGTACCCGTCTGCAGC |
| | CTGGGGACTGGGGACCCTGCTTTAGATGATGTACTCTGGCTTTGCATTCTGGC |
| | ATTAGCTAAGCACCCTCTTAAAGGAAATTGGGTCTATACTCTCAGTCCGTGTT |
| | CTCCCTAACACCTGGAAACATTGAATACCTTCAATGCTGGGAAGTTAACTCC |
| | CACCACAACTAGAACAGCTATGGGAAAGACAACAGTTGATTTTGAAGAGTGT |
| | CACCAATTTGCACATGATTCCATCCTTAACCATTCTTATCCTATCAGCTCTGC |
| | CAAACATGGAGAATAGTTGGCTGCAGGACAGCTATTTTTCCTACTTGTAGAT |
| | GCAACTATTTCTCACCCACCAGGATGTAAAAGGTCCCTGTACCCTAAGATTG |
| | GTCCTACATACACACCCAATGGGAAAATGAGATGAAAAATTTAAAGCAGTA |
| | AATATTTGAGGAAGTAGATAGAGTAATTTAGAAAAAGAAAATACACAGGGC |
| | CAAGCACAGTGGCTCACATCTGTAATCCCAGCACTTTGAGAGGCCAAGGTGA |
| | GAGGATTGCTTGAGCTCAGGAGTTTGAGGCCAGCCTAGGCAATGTAGTGAGA |
| | CCCCACCTCTACAAAAAATTAAAAACTTAGTTGGGCTTGGTAGTATGTACCT |
| | GTAGTTTAAGAAACTTGGGAGGCTGAGCTGAGGCAGGAGGATTGTTTGAGCC |
| | CGGGTGGTCAAGGCTGCAGTGGGCCATGATTGTGCCAGAGTACTCCAGCCTG |
| | GGTGATAGAGTGAGACTCTGTCTCAAACAAAAAAAAAAACAGAGACAGAAA |
| | AAAAGAAAGAAAATATATGGATGTATATCATATAAAAATATAAATAAGGGA |
| | GGCCAAGTGCAGTGGCATGCCTGTAATCCCAGCACTTTGGGAGGCTGAAGCA |
| | GGAGGATCACTTGAGGCCGAGAATTCGAGACCAGCCTGGGCAACGTATTGA |
| | GACCTCATCTCTGCAAAAAATCAAAAAATGAGGCGGAAGGATGGCTTGAGC |
| | CCAGGAGATCAAGCCTTCAGTGAGCTGTGATCGTACCACTACACTCCAGGCT |
| | GGGTAACAGAGAGAGACCCTGCCTCAAGATAAATAAATTCATACATACATAC |
| | ATACATACGTACATACATACATACATAAGAAGACTTGTTTCTTTCCATTTGCA |
| | ATGTTTCATTCAAAGGCTAGAATTAAATTGCCGTAGGCCATCACAAGTTTAG |
| | CTTGAATATTATTATTTTTTCAAGATGGAGTCTCACTCTGTCACCCAGGCTGG |
| | AGTGCAGTGGTGTGATTTTGGCTCACTGCAACCTCTGTCTCCCGGGTTCAACC |
| | GATTCTTCTGCCTCAGCCTCCCATGTAGCTGGGATTACAGGCGCCCGCCACCA |
| | CACCTGGCTAAGTTTTGTATTTTTAGTAGAGACAGGGTTTCACCATGTTGGCC |
| | AGACTGGTCTTGAACTCCTGACCTCAAGTGATCTACCCGCCTCACCCTCCCAA |
| | AGTGCTGGGATTATAGGCTTGAGCCACTGCACCCAGCCTAGCTTGAAGAAAA |
| | TTTGATAGGAGTTTGTTTTTTTCTATTTATAGGCCAAGCAATACCACGTATAA |
| | ATATTAAGAATCATGGCTGTTCCTTAGTGCCTAGTTGTTTATAAACCATGGGA |
| | AAGAATGAAATCATTGACCAAATGAGACAGGGTAACAGTCTTCCCTGGGAGT |
| | AAAGAGACTAGCAGTCTCTATTGACATATATTTTAGGCCTGGCCTCCAAAAT |
| | AAATTTACCCAAAGAAGTGATGTATTTGTGTCCAGGGCTACCGAGCCTAATC |
| | TTTGGCTGCCTCTGTGTTATTCTAAGTTGTAATTTTCCATGTCATCTAAATTTG |
| | TAATAATTCTTTAATATGATAGTGTTTCAGTGAACAAACATTCTGCTTGCTGC |
| | ATTTCTTCTGAGTGAGTAATTCCCTGACAACTACCAGATCTTGGCAGAAGCA |
| | AAGTTGGTCATAAGTTATGCTCCACTCTCAGTGCTGGTGAAACGTATGATGC |
| | GTAACACAGTGTTTTTGAATTCAGTGCTGATTTTCCTAAAGGACATTTGGAAG |
| | GAAAAAAGAAAAGGAAAGGAAATACCCAAATTCAGGAATAGAACTTACATA |
| | TTATTATAAGACTTAAAAAATACATGAACACTAATGATGAACTCATTTCTTCG |
| | AAGTAAAAGGCCTTATGCTATTTTTTCCCATTTCCCTATGTGGCTTGATTGTG |
| | GCGAAAGTGGCTGTGTGAGTTTCCATTATTGAAGGAGTTAAGGTCTGTGGAA |
| | TCAAAGCATGAGACAACATGCAAGGACCAGGTTGGTTTCCATTTAACAGCCA |
| | ACCTAATCTAACTGAAAGGATTGAGAGGTTTGTCTTTTTTGGAAAGTGTTAA |
| | GGTTCTTCCAAGTAACCAGCAATGTGACTTTACCACACTGTTCATTAACGGG |
| | GCTTTGGATGGCCACCATGTCTGCTGCTGGCTGAGTCCAAAACTGCGGTCAC |
| | TCTCTCCAGCAAGCTCATGGAGGGTTACGTCATCTTCACTCAGCCAGCCCAA |
| | CGCTTTTTCCATCTGCTAAAATGTAGAACATGGTTGTTGATTCTACTCTTTCTA |
| | CAAAGAAAGAGAGGGAACATGGATGAGTTGCTGCTTTTAAAGATTATATGTT |
| | AATTGCTGTTTTAAAAATCTGCTCAGCTAAGCACGCTTAGTGTAATCAGTCAC |
| | CATGGAGTTTTTTAATAGGACAGCTTCTGCTCTTACAGAGCAGAGTTTTTATG |
| | CCAGTAGGTTAGAAGCATAACATGTTTCTATATTGAAGTGATTCTCCAACAA |
| | GGACTTCTTTTCATCAGGGGTGACCTAAAAGTTATTACTTGAAATATAATTAC |
| | ATCATGTTCTTAACTGGGTTTAGTAGTAGGATAATTACAAGGTTGTATCCACC |
| | TCAAATAGGAAGAACTGATAAGTTTTGCACAATTATTTAACTCCTCTGTTAGG |
| | CCTTCCTTATTTCCTGTTCTCTTTTTAAAATTTGACTAAGATATTGCTAATGGG |
| | CTTGGGAGCCTATAAAATGATCAGAATTGTTGCCTTATGTTTTGCATGTTTGG |
| | GGTAACATTGGAGCCAGATGTACTCTTAAATAAAAGGTAGGCCTACAAAACC |
| | AGTTTCTCAGTTGCATTCAAAATGTGATTAAAAAAAAAAAAAGGAGAATCTC |
| | CCTTATAGGTGAACTTTTTAATTTGTGCTTTATTTTCCCTTTTGCCATTCATGA |
| | GATTTCTTAAATAAAATGATATCTTTTCTTTTCTTCATTATTATTTTAAAGGTC |
| | TCTGGGGAACAAGACTCATGGAGGAAAGCAGCACTAACCGAGAGAAATACC |
| | TTAAAAGTGTTTTACGGGAACTGGTCACATACCTCCTTTTTCTCATAGTCTTG |
| | TGCATCTGTAAGTAGAATATTTCCTTGCACTAATGGGAAAGTTTTGAAAAGA |
| | TTTGACCTATCCAAATCATAATTAAAAGGAAGTGTGTATGCACCAGAGGGGC |
| | AACTGGGAAGTTACCTTCTTACCTTTGTTTTTAATTCTAATATTTTTATTTGGG |
| | CATTTGTTTATTGACTATCTTCCTATGGTAGAATGCAAGCTTTATAAGAGAAG |
| | GGACGTGATTTGTTCTCTGCTGTACCCCCATTTCCCAAAACTGCAGATGGCAA |
| | CAGAAGGCTCTGAAAAATATATAAGAAAGAATTTTTCTAATTGTGACTAAAT |
| | TGTGACCAAATGCTAAGTGACTGTGGACTTGCGTTTAACACAGGACGGGAGA |
| | GGCAAAGAGTTCAATTCCAATTTAGAATTTGGTCAAGTTCTCTTCTGCACTCT |
| | GGTAAACATTAATTAAAAATCAGCATTATCTGACCAGCCAGTTCATCGTCAG |
| | TGGTGGTGATTTTCACTATGAGATACGCGTGGCAACTTGCCAGACACCAAGA |
| | AACCAAGTTAGAGGATTTTTGTATTAGATTCCTTAACAATGAATACAGTATC |
| | ACCATTATTACAGTATCATCATTATTGTCATACTATTATTATATCAGTTAACA |
| | TAAAGTCTGCATAAGAATTGTTTCCAGAAAAATGACTTTCCAAATTTAACTTT |
| | CAGGAAATACAAATAATGCTACTAATATTGCTTTTATTGGCGTATACATGTA |
| | ATATCCCCTTCTTTTGGATTTGGATATGTTGTGTCATTGCCTCATTTTAATTCA |
| | TTATTTCTTCTCAATCTTTAATAATTGCTGGACTTTTACTCCACAAGAAACTTG |
| | CTATAGGCCCATCTCTTTCGTCTTCTTTCCTTCTTTCAGTTCGTCTTCCCATCCT |
| | CTGGTAGGGGGAGGGGAGGGATGCCTGAGCGAGAGACTAGCTGTAGGAACC |
| | ATTTGTCTCAAAGTCCAGAAAGCCACAGGTGATGGATTTGTCCTCTGAATCA |
| | AAGGGCGTTCGATGATGGATTTCTGTCATGTCTCATCTAAAGTCTTCACGAGA |
| | ACAGATGAGGAAGCAGTTTTATGACCCCAGAGCCTCCTACCAAACTCCTCTG |
| | AGAAAAGGTTTCCTTTTTTTTTTTTTTTTTTAAATTAGAGACAGAGTCTTGATC |
| | TGTTTCACAGGCCGGAGTGCAGTGGCACAATCATAGCTCACTGCAGCCTCGA |
| | GCTCCTAGGCTTCAGTGATCCTCCCACCTCAGTCACCCACGTAGCTGGGACTA |
| | CAGCTGCACACCACCATGCCCAGCTAACTTTTAAAACATTTTTGTAGAGGTG |
| | GGGTCTCACTTTGCTGCCTATACTGGTCTCCAGCTCCTAGCTTCAAGTGATCC |
| | TCCTGCCTTGGAAGTTCTGGGATTATAGGCATGAGCCACTGCACCCAGCCTG |
| | GATGTGATATTTTTATGTTTTAAATTGTTAGAGTTTAGAAACTTGAGATTGAG |
| | TTTGCTGCCTGCATTAAAATGATGCTTAAACATTAAACTGCAGTGGCCTTAAA |
| | TATTAACAAGTTGATTAGAATTACTAAGTTCTTTTCAAGCTTTACATATACAG |
| | ACAAATTTCTTATGCAAAATAGAAGGTAACCCCTGTACGTAAGTCTAGAATT |
| | TCAGCAGTCCCCAAAACTGACTGAGCATTAGAATCACTCTGATTTTAAAATA |
| | CATATGTGGTTTTCCGAGATCTACTAACAGAGCCTCCATAATGTAGCCTAGA |
| | GAAACGAGTTTTCAGATGTAGTGATAAATTTGGAAGGTAAGTCAGAGAAGTA |
| | AGCTGAAGACAGAGTTTTAGGAAATATGCCTAAAGTCACATAATGAATTGGT |
| | TTTCTTGTTTATTTGAGAATATTGTCGCTTTTTGTTCTTTTTCATGCAAATCAC |
| | ATTTTATTTCTTATGTGAGTAGCTATATATTTAAAAATTTTGTTTTTGGAATAT |
| | TGTAGAATCTCTACTTAAGAAAGTATCTTAGCAGTCATATGGTCTGACCTCAC |
| | TGAATGCTAAATTCTCTTTAAAACATCCGTCTCAGATGGTTACTCATACTGCC |
| | TCTGATTGAACAAGAGTCCCAGGTTAAGGGACTTACTTCTTTGAAATCGTTCA |
| | TTTCATTTTTCTACAGCTATGTTAGAAAGTTCGTCCTTAGCAGTGAAGCCAGA |
| | GTCTATCTCTTATAACTTCTACCCAGTTGCACCCTCCAAGCCTACCTATACCA |
| | AGTATCTTTTTTCCACGTTGCTTTTCCAATTCAGCTCTTCGCAAGTTTGGAGAC |
| | AAATACCTAATCTCCTCTAAGCCTTCTCCAGGTTAAGCCTTTCCAGTTCATCC |
| | AGCTGTTGATTATGTGATTGGAGACACAAGTTTGAGTAACTCCCATGATGGA |
| | AAGTCCCTCTGGTGAATGTTCTGTTCATCAGAGTCCCTAAGAAAGCACATGA |
| | GCCTCACCATGGTGAGTGGGGCCATGAGATTCCTAAACCAGACACTACACTG |
| | TGGCTTGTGCAACCTACCATGGCCAGACTCATGAGGCTGTTTCATCATATAA |
| | ATAATTCCTTAGTCTCTTCACCAAAAACTGTGAAGCACTGTGTCCCCCAGCTG |
| | TATGTGAGCTAGCCTGGGACCATAGTGGAGGACTCCTCTTTCAACCCTGTTA |
| | AATTTCATCTTGTTAGGATCTGCCCATTTTTCCATTCTGTTGAAATTATCTTGG |
| | AACTGGATTCATTCATCTCACATCAGCTACTCCCCGAAGCCTCACAGTATCAG |
| | CAGAGTTATTATCTCTATCCATATCCTTGTAAATTATTAAACTAAAAAAGATT |
| | GGTCTAAGAACATCAGTCCATTAATCAAGGCTCTTTGGTTGGAGTTCACTTCA |
| | TTATATTATCATCCAGCTCAGAGTGTTCATAAACAAGATGATAGGAAAGACT |
| | TTTCCAAATGCCTGTCTGAGTAATTCCCCCATTCCTGTGATCTGTGAGCTGTT |
| | GACCAGATTAAAAAGGAAGTGAGAATAACCAGGCAAGATTTACTCCTAGCA |
| | AAACCTTACTGGCTTCTAGTGACTGAGTCCTTTCCCTATTGCTTACCAGCTAT |
| | CTTTTTAATTACTAGTTTTAAAATCTTGCCAGCAATGTCAATATCAAATGACC |
| | AAGAATATCAAACTCATTACTCTGTATGTAAATGAGATAGTGTACTTACCCCT |
| | ATGGCTTAAGTATTAGGCTGTTCTTGCATTGCCCTAAATACCTGAGACTGGGT |
| | AATTTATAAAAAAAGAGGTTTGGCCAGGCACTGTGGATCAGGCCTGTAATCT |
| | CAGCACTTTGTCAGGCTGAAGCAGGTGTAATGGTGAGCCAAGAGTTCAAACT |
| | TAGCCTGGACAACAAGGTGAAACCCCCTCTCTGCAAAAAATACAAAAATTAT |
| | CTGGGCATGGTGGCATGCACCTGTAGTCCCAGCCACCCAGGAGGCTGAGGTG |
| | GGAAAATTGTTTGAAGCTGGGAGTCAGTGATTGCAGTGAGCCATGATTGCAA |
| | CACTGCACTCCATCCAGCCTGGGCGACAGAGCAAGACCCTGTCTCAAAAAAT |
| | AAATAAATGAATAAATAAATAAAATAAATAAATAAATAGAAAAGAAAAAGA |
| | AAGAAAAGAGATTTATTTGCCTCATGGTTCTGCAGGCTGTAAGGGAAGCATA |
| | GCTCCAGCATCTGCTTCTGGGGAGGCCTCAGGAAGCTGTTACTCATGGCAGA |
| | AGGTGAAGCAGGAGCTTGCACATCATGTGGCAAAAGCAGGAGCAAGAGAGA |
| | GAGAATGGGGCAGGGAAGAAGCCCCACACTTTTAAATGACCAGATCGCATG |
| | AGAAATCACTCGTTACCTCAAGGACAGTACCAAGAGGATGGTACTAAATTCC |
| | TGAGAAATCCACCCCCATGATCTGATCACCTCGTACCAGGCCCCGCCTTCAG |
| | CATTGGGGATTATGTTTCAACATGAGATTTGGATGGGGACAACATCCAAACT |
| | ATATCACCTTGCATAGTAGGTAGGGTTTTAAAAAGCAGTTTGGCACAGTAAG |
| | AAAAGTACAGATTTTTTTTGCATCAGACAGACCTGAGTTAAAATCCCAGCTT |
| | CACTGCTAACATGCTAGGTAAATGTGGGCAAGTTAATTAACATTTCTAAGCC |
| | TTTGTTTCCTCACTGGTAAAACAAGTATTTGGAAATATCATTGTGAAGATTAG |
| | AAATAATACATGAAAAGATCCTAGGATGCTGTCTGTCATACAGTAGTAGTAG |
| | TAAGAAGTTATTCTTGCCAAAGATTGTTGAGAATGGCAGAATTATCTCAGTT |
| | CTAAGAGCTATAGTTTCTAATTATTTGAGCCTAGACTCAGATTCATTTGGAGC |
| | AGCTAACTGCTCACCAAGAGCTTATTTTCCATCTTACCAATGAGGTTATGTGC |
| | CCTGTGTTTTTAAAATCAGTCTACTTAACCAAGAGAACAGAAATGACATGAG |
| | AATTAAGTAATCTCACTTTCTCTGTTATTTAGGATTTATTCCTACTCAAAACCT |
| | GAGAGTTGCTATGAATTCACCATTAAAGCACTTATTAATATACATGGGTTACT |
| | GTTATAAATAGCAATAGTATTGCTATTGTGTGAGTTAGGTGTTGAAGTTCAA |
| | GAAAGGAATAAAGAATATTTAGAAGATCTTTGAAAACAGTGTCTGGGTACG |
| | GTGGCTCATGCCTGTAATCTCAGCACTTTGGGAGGCCGAGGCAGGCAGATCA |
| | CTTGAGGTCACGAGTTCAAGACCAGCCTGGGCAACTTGGCGAGACCTCGTCT |
| | CTACAAGATATACAAAAATTAGCCGGGTATGTTGGCATGCACCTGTAATCCC |
| | AGCTACTTAGGAGGCTGAAGCACAAGAATCACTTGAACCTGGGAAGCAGAG |
| | GTTGCAGTGAGCCAAGATTGTACCACTGCACTCCAGCCTGGGCAATAGAGCA |
| | ACACTCTGTCTCGAAAAAAAAAAAAAAAAAAAAAAAAAGAAAGAAAGAAG |
| | GAAGGAAGGAAAGAAAAAAAAAGGAAAAAATGCAAGGAAGGTATTTGGTG |
| | AATCTATAATAATAAAAATGTATTTGTCATTTCCTTTTTCTGTGCTCTCATTCT |
| | ATAAAATTGAGTAAAAAATCTATATATAGTTTAAACACATTAATAGAAATCA |
| | CAAAAGTTAGCTGAGTCAACATTGTAGAAACATAATATTTCTGTATGTCAAA |
| | GAAATAGACAACATTAAAAAGCAGAAAGCAATTACAAAGAATGATTATAAG |
| | AAACATCACAAAGGGTTAATATTTTAACACATTTGAAACTCAAAAATCACTG |
| | AGAAAAGCAGTAGACTTCCATAAATATTTTATAGAGTAGAAAAAATAGGCC |
| | AAGCACAGTGGCTCATGCCTGTAATTCCAGCACTTTGGGAGGCCGAGGAGGG |
| | TGGATCACGAGGTCAGGAGTTCAAGACCAGCCCGGCCAAGATGGTGAAACC |
| | CCATCTCTACTAAAAATACAAAAACTAGCCAGGCGTGGTGGCAGGTGCCTGT |
| | AATCCCAGCTACTTGGGAGGCTGAGGCAGGGAATTGCTTAAACCCTTAAACC |
| | CGGGAGGTGGAGGTTGCAGTGAGCCAAGTTCGCACCACTGCATTCCAGCCTG |
| | GGCGACAGAACGAGACTCTGTCTCAGAAAAAGAAAAGAAAAGAATAGAAA |
| | AAGAATCCATGGGCAGGCACAGTGGCTCATGCTTATAATCCCAGTACTCTAG |
| | GAAGCCAAGGTGAGAGGATCAATTGAGGCCAGGAGTTCAAGGCCAGCCTGG |
| | GCAACATAGCAAGACTTTGTCTCTATTAAAAATTTTAAAATTAGCCAGGCAT |
| | GGTGACGCACACCTGTAGTCCCAATTACTTGGGAGCCTGAGGCAGGAGAACT |
| | GCTTGAGGCTGCAGTGAGCTATGATTAGACCACTGCACTCCAGCCTGAGCTA |
| | CACAGTGAGACCTTGTGTCAAAAAAGTAAAAAAATAAAAATTAGCCAGGCA |
| | TGGTGGCACATGCCTGTAGTCCCAGCTACTCAGGAGGCTGAGGCAAGAGGAT |
| | GACTTGAGTCTGGAAGATGGAGACTGCAGTGAGCTGTGGTCATGCCACTGCA |
| | CTCCAGCCTGGGTGACAGAGCAAGACCCTGTCTCAAAAAAAAAAAAAAGAA |
| | AAGAAAAGAAAAATAAATAAAATTTATTCAAATACAAAAGTGATGTGGTTT |
| | GACTCTGTGTTGCCACCCAGATCTCATCTCCAATTGTAATCCCCGTGTATTGA |
| | CGGAGGTTCCTGGTAGGAGATGATTGGATCATGGGGATGGTTTCCCCTCTGC |
| | TGTTCTCATGATAGTGAGTGAGTTCTCATGAAATCTGGTTGTTTGGTAGGTGT |
| | CTGTCACTTACCCCTTCTTTTTCTCTCTCCTGCTGCCTTGTGAAGAAGGTACTT |
| | CCTTCTCCTTTGCCTTCCACCATGATTATAAGTTTCCTGAGGCCTTCCCAGCC |
| | ATTTGGAACTGTAAGTCAATTAAACCTCTTTCCTTTATAAATTACCGAGTCTC |
| | AGGCAGTTTTTTATAGAAGTGTGAAAATGGTCTAATACAGAGACTTGGTACC |
| | AGGAGTGGGGTACTGCTATAAAAAATAACCTGAAGATATGGAAGCGACTCT |
| | GGAACTGGGTAACAGGCAGCAATTGGAACAGTTTGGAGGGCTCAGAAGAAG |
| | ACAGGAAGATGTGGGAAAGTTTGGAATTTCCTAGAGACTTGTTGAATGGCTT |
| | TGACCAATACACTGATAGTGATATGGACAATGAAGTCCAGGCTGAGATGGTC |
| | TCAGGTGGAGATGAGGAACTTATTGGGAACTGGAGTAAACGTCACTCTTACA |
| | TGTTTTAGCGAAGAGACTGGCAGCATTTTTCCCCTGCCCTAGAGATCTGTGGA |
| | ACTTTGAACTTGAGAGACATGATTTAGAGTATCTGGCAGAAGATATTTCTAA |
| | GCACCAAAGCATTCGAGAGGTGACCTGGCTTTTCCTGAAAGCATACAGTTAT |
| | ATGTGCTCACAAAGAGATGGTTTGAAATTGGAACTTATGTTTAAAGGGGAAG |
| | CAGAGTGCAACAAAAGTTTAGGGAGTTTGCAGCCTGACCATGTGGTAGAAA |
| | AGAAAAACCCATTTTCTGGGGAGAAATTCAAGCTGGCTGGAGAAATTTGCAT |
| | AAGTAACGAGGAGCTGAATGTGAGTTGCCAAGACAATGGGGTAAATGTCTC |
| | CAGGGCGTTTCAGAAAATCTTCAGGGCAGACCCTCACAACACAAGCCTGGAG |
| | GCCTAGAAGGGAAAAATGGTGTGAGCCAGGCCCAGGCCCAGGCCCCAGCTG |
| | TTCTGTGCAGCCTTGGGACATGGCACCCTGTGTTCCAGCCACTCCAGCTCCAG |
| | CTGTGGTTAAAAGGAGCCAAGGTACAGCTGGACCATTGCTTCAGAGGGTACA |
| | AATCCCAAGCATTAGCAGCTTCCATGTGGTGTTGGGTCTTTGGGTGCACAGA |
| | AGACAAAAGTTGAGCTTTGGAAGCCGCTGCCTAGATTTCAGAGGATGTATGG |
| | AAACACCTCGATGTCCAGGCAGAAGTCTGCTGCAGGGGCAGAGCCTTATGGA |
| | GAACCTCTGCTAGGGCAATGCAGGGGGGAAATGTGGGGTTGGAGCTCCCAC |
| | ACAGAGTCCCCACTGGGGCACTGCCTCATGGAGCTGTGAGAAAAGGACCAC |
| | CATCCTCCAGACTCCAGAATGGTAGATCCACCAACAGATTGCACTCTGCGCT |
| | TAGAAAAGCTGCAGGCACTCAATGCCAGCCTGTGAAAGCAGCTGCAGGGGC |
| | TGTACCCTGCAGAGCCACAGAGGTGGAGCTGTCCAAGGCCATGGGAGCCCA |
| | CCCCTTGCATTAGCATGGAGACAGGGGATCAAAGGAGATTTTGGAGATCTAA |
| | GATTTAATGAATGCCCTGTCGAGTTTCAGACTTGAATGGGGCCTGTGACCCCT |
| | TTGTTTTGGCCAATTTCTCCTATTTGGAATGGGAACATATACCCAATGCCTGT |
| | ACCCCCATTGTATCTTGGAAGTAACTAACTTGCTTTTGATTTTACAGACTCAG |
| | GCAGAAGGGACTTGCCTTGTCTCAGATGAGACTTTGGACTTGAACTTTTGAG |
| | TTAATGTTGGAACGAATTAAGACATTGGGGTTCTGTTGGGAAGGCGTATTTG |
| | GTTTTGAAATGTGAGAAGGACATGAGATTTTGGAGGGGCCAGGGGTAGAAT |
| | GATATGGTTTGACTCTGTGTCTCCACCCAAATCTCATCTCCAATTGTAATCCC |
| | CATGTGTCAAGGGAGGGACCTGATGGGAGGTGACTGAATCATAGGGGCAGT |
| | TTCCCCCATGCTGTTTGCATGATAGTGAGGGAGTTCTCATGAGATCTGGTTTT |
| | TTGGTAAGTGTCTGGGCTTCCCCCTTTTCCCTCTCTCTCCTACTGCCTTGTGAA |
| | GAAGGTACTTGCTTCTCCTTTGCCTTCTGCCATGATTGTAAGTTTCCTGAGGT |
| | CTCCCCAGCCATTCAGAACTGTGAGTCAATTAAACCTCTTCCTGCCTATTCTC |
| | AGGCAGTTCTTTATAGCAGTATGAAAATGGACTACTACAGAAAGTGTGTAAC |
| | TTTAAACTCAGTAGTATCCAAAGAAGTAATGAAAATGGAGAAACGAACAAC |
| | AAAATCATAGTACAATATGGTGTATGTACTAGGACAGGAAGAGCCCTTTTAA |
| | GAAGAGATCTATGTATTTCCATTTGTTTATCTCTGAAAGAAAGCAACTTTGCC |
| | TTGTATTCTGAAAAAGAAAGGAATATTTTATTTTACTTGTAAAAATCTTACAA |
| | GGATGCTAGTCTAAATATAGTTTTCCTAATTTGCCAGAGAATCCATGAAGAT |
| | CGAGTTGATAACAAGATCAGTGAAGTAAAGGTCAGTGAGTTAATCTCACAGC |
| | AGCTGCAGGCTAATTCCATTTCCAGTGAAAAACGTCTTGATTGCTCACCACAT |
| | ATCTTTTCACCACAAACAGTTTCAGTCTTAAGATCACATGTTGCAATCCATGA |
| | GAAGTAACTATTAAGCCTTCAACTATGACTGGAGGGCTCCTCGCCCTTTCTGA |
| | TAAATTGACTGGACAAAAACTCAATTTTAAAATGACAAGAAATAGAAGATGT |
| | ATAAATGTACTTTAAATGTGACCAAAATGGGTTGTGAAAACACAAGACACAA |
| | TATCCAAAAATGCTGGCAACACAGTACACTGTAGAGTATTGGTTGTTTATTTA |
| | CCCTTGCTATTGTGTGGCTGAGCTTACTGCCACTGCCCAGCATTGCAAGGGCA |
| | TCAAACTGCCTATCACTAGCCTAGGAAAAGATCAAAATTCAAAATTCTAAGT |
| | ACAGTTTCTACTGAATGCTTATCACTTTTGCACCATTTTAAAGTAAAAAAATC |
| | AGTAAGTTGAACCATCATATATCCAAGATTGTCTGTATATAAATATTATACAT |
| | CTTTCTCTCACTTTTAAAACAAAATAATACTAGCCAATACTACCATTCTCAAA |
| | AGCACTTGTGTCAACAGCCTTTACCCCTTAAAGATTTTCCTCACAATTTTAAA |
| | ATTGTTACTTACTATTTTCTTTGAAATGTTGACCAAACCTGGATTAAAAGATT |
| | TGGGGGTTTTAGTGACTGTATTTCACAAACTCTCTTATTGATTCTGCAGCCTC |
| | ACTTCTGCCTCCTAAAAAGCCCTCACCAAGGTCACGGGGGATGGCTCTTTTC |
| | AGCCTCTTCCTGGCATTTGGTCCAGTGGCATTTGGCATTCTAGGACTTCCCTC |
| | TTTGTCTTTGATAACTCCCTCTCTTCCTGTGTTCCTCCTTGCTGTGTTCACTTG |
| | CTTCGCTTTCTTCTTTCTGAAGCATGTTTACACAGTGTTTTCTCTGATTGGGCC |
| | TGTGACGTTCTTTAGGTCATCTTTTCCACAAATAATGCTTCAACTAGTACTTG |
| | CGTGCCAGTGACTCCACGTCCCACTCATGAGCTCTGAACCTAGTACCAGCTTC |
| | TGCTGGACATCTACAATGGGATCTCTCACAGGCCTCTCTCATTGGTAACATGC |
| | CCCAGCCTGAACTCATCTCCCACCCATCTATCCAGCCATGCTCTCTAGTTCAC |
| | CTGAACACTTGGGTGTCATCCTAGATGCTTTCCCTTCCCAGTCTTCTGTGATC |
| | ATTCTGCCTCATCAGAGGCTCTCTAATCTGTCTTCTTTCCTATATCGCTCTTGT |
| | CCCTATTTTAATCCTAATCATCTATTTCCTGACTTATTCATTCCTTAAGTTGGT |
| | CAGTAATTTAATTAAAAACAGATTTAGGCCCTGACCTTAAATGTGATAAGTG |
| | ATATGAAAGGAGATGACTGGGGAAAAGGATTTCCCTCAAGGAAGGCCTCTG |
| | TGAAGCCTGAAGCAAGAATGAAAACGAGTCAGACGAAGAGAGAATTGTATG |
| | AATGAAGGCTCTGAGGCAGGAAAACACTCAGATCATTCCAGAATCACTTAGA |
| | AGCCAAGTGAAGCCAGTTCCTGGAGAGCAGATCATCAAATGAAGATGGAAA |
| | GGTGACCAGGGGCCAGACCTGTAGTTTTGGTGGGCCTTGGTGAGGGATTTAC |
| | AGTAGGACACCCCATGGTTTAAGTATGAAAGTGACAAGATTCCTTTAAGTTT |
| | TAAGAGGCCTCGAAATATGAACCACAGATTAGATGGAAGCTACTCTCCCTGT |
| | GTCTGGACTTTTTAGAATTTCCAAGAGCTGCTGTTTCTGGAACCAGATTAATA |
| | CAAGTCAGTCTTCCATTTATTTATTTATGTATTTATTTGAGACAGGGTCTCACT |
| | CTGTCACCCAGGCTGGGGTGCAGTGGCATGAACACAGCTCACTGCAGCTTGG |
| | GGGCTCAAGAGATCCTCCTGCCTCAGCCTCCCATGTAGTTGGGACCACAGGC |
| | ACCTACCACCCAGCTAATTTTATTTGTTGTAGAAATGAGGTCTCATTTTGCTG |
| | CCCAGGCTGTTCTTGAACTCTTGGGCTCAAGCGATCCTCCTGCAACATCTTCC |
| | CAAAGTGCTGGGATCACTCTTCCATTTAACATGCTATCTCAACGTCAAGATA |
| | AACTTTAAAATCTTTAGATAATAGGCTGGCATTTTACTTAAACGATCTTTACT |
| | TCTTCAGAACTGCCATTCCCTATAAATATCTGGTTCTTCAACCACATCAAACC |
| | ACTTGTGATCTCAAAAAGCCTCAGCGTACACTGTCCTTTCTGTCATTCTAATT |
| | CCTCCTCATCCTTCAAAATCAACTCAAGGACCAGATCCAGGGAGAAGCTTAG |
| | TGGTGCCCACCCGAACCGGCCCCCTCCTTCGAGTTGTGCTGCCATTCGGGCCC |
| | ACCTCTTCACACAGGGTTGTCAGACCAGACCAGCTCATGCGTTCACCGCCCTT |
| | GCAGGGATGGGATGCAGCTGTGCACCTCTCAGTGCCGACACCTGGAGAGTCT |
| | CACGAAATGTTGACAACATGGCCTGTTTCCATTTCTTGTTCACTAGGACTTCT |
| | CATTTACTAACACACAGAATTTCCTGTAGTATGTCCACTTAATCAGTTCAAGC |
| | CTAATAATTCCTTGATTTGGGTATAGTGCTTTGCATTTATATACTGATGGTCC |
| | CCAACTTACGATGGTTCGATTTATGATTTTTCAACTTCATGGTGATGTGAAAG |
| | TGATACACATTCTATAGAAACCACACTTTCAATTTTGAATTTTGGTCTTTTTCC |
| | AGGCTACCATACTCTAAAGATAGAGCCACAGATCCCAGTCAGCCATGTGATT |
| | ATGAGGGTAAGCGACCAATACTCTACAGTGTATTGTATTGCCAGATGGTTTT |
| | GCCCAACTAGCCTAATGTAAGTATTCTAAACATGTTTAAGGTAGGCCAGGCT |
| | AAGCTGTGTTGCTCATTCAGTAGGTTAGGTATATTAAATGCATTTTCAACTTA |
| | TGATATTTTCAATTTACAATGAGTTTATCAGGATGTAACTCTACTATAAGTCA |
| | AGGATCATCTTGTATAGCACTTTTAATTTATAGAGTCCCTTCAAATGTTTGTT |
| | TGTATTTTATTTCCACTGCATCCCTGTGAGGATACCATAAGTTATACAGCTAA |
| | CAAAACAGTTAGTTTTCCTGTGCAAAGTGATGGCTTCATCTTGTGGCAGATTA |
| | CCTGGAATACTGTGGCCAAGGCATCTTAGTTCTACTGTCTTTATATATCTAGT |
| | ACAGTTATATTTTTATGGCAGCTCTGATTTCTTCTTTGGCCCAAGGGTTATTA |
| | AGAGAGGGAAAAAATTTAATTTCTTAACAGATATATATATCTATGTCAAGTC |
| | ATATATTTAATTCAAACCCTTAATATTCCTAGGTAATTTTTGTCTACTTTCTCT |
| | GTCAAAGATTGAAAGATACAGGGTTTTAAGTTTCCAACTGTAATTGTAGTTTT |
| | GGTAATTGTTTTATTACTAACAGGTATTGCTCTGTATATTTTGATGTTCTGTTA |
| | TTCAATACATAAGAATTCATTACAGTTGAATCATCATGTATAGTATAATTTAC |
| | CAATGTAAAATAACCTTTTTATCAAACTGAGTATTCACCTTATAATGTCCTCT |
| | GTCAGAGGATTATAATACCACTTAAAACCTTTTAAAAAATATTTTGTTCCTGA |
| | TAATTTTAGCTTTGAGTGGTTTTCTTATAACAATTCTAGAGATGCGTTTTTATT |
| | TTTTAACCAAGATTTAAAGCTTTGTGATTTAGTAAGAGTCTAAACCATTCACA |
| | GTTCATGCTTTTTTGTCAAATTTCTATTATTTAGTATTTTCCTCTCTTTTATATT |
| | TTCCTGTTATTTTCCATTTCTATTCTTTTGTTAGACTGGAAATTTTTTCTTTGCT |
| | TTCTTTTATCCTAGTGATTTGAAATTTATGTAATATATACTATTCTACAATACC |
| | CTTTATTTGTTTTCAATATTTGAACCTATATTTTTCAACATTATTAAGAATAAA |
| | ATAGTATTTGTTGCTATTTTGAAATGATAGACCATGTTTTTAAGGCAGGTTGG |
| | TGGTTGTTAAGGCACCAGTATCGGCCAGGCACGATGGCTCACACCTGTAATC |
| | TCAGCACTTTCGGAGGCCGAGGTGGGCAGATCGTTTGAGCCCAGCACTTTGG |
| | CCGATACTGTGGTTTACTGTATTTTGTTCTAGTTTATTTTATGGAAAATGGGA |
| | ATTCAGTGGTTAAGACAAGGATTAAATAGCAGAAGAAAGATGTGTACATAT |
| | GTACAGATGTATGTGTCCTTTATATGTTTTTTAGTACTCTTGTCTCCTTTCTGG |
| | TCCTCATTTAAGGTTATCTATTTCATGCAGTAAATTTTTCTTCACAATTCATTT |
| | CATTTAGAGAGTGAATGCTACCTTCCAAGTGGGCTTTCTCCAGTTTTCCTTTC |
| | AGGGACTTAAAGGAGAAGTGATGTTAACAGTTTTATATTTCCATTGCATTTTA |
| | CAGTGTGCAGATGTCTTCACATATATTTCCCCATTTGAGCTTTACAAAAGCCC |
| | TTAGTATTATTCTCATTGTCTAGATTCCAAAATCAGGCTTAGAGGAGTTAAAT |
| | AGTTGTCCAGGATCTCAAGATGCAAGACCCACAATCATGAACAGAGGCAGA |
| | TGTTCAGGATGGAGGCAAGCTGAAACTCAAAACCAAATCATTATGACTCCAA |
| | ATTCAGGAGTCTTTTAGCTGCCACCTGCATGGGCTCTTGGTGTAGCTGACCAC |
| | CAGAGTTTGTAGAGCTGTCATTCAGGTGTGCCATGGACTTTCCTGGGACCTG |
| | GCACAGGAGAAGGACTGAGTTAATGTTTGCTGATTAAATATCTGTTACAGGC |
| | TGGGCGCGGTGGCTCACGACCGTAATCCCAGCACTTTGGGAGGCCGAGCAGG |
| | GAGGATCACTTGAGCTCACAAGTTTGAGACCAGCCTGGGCAGCATGGCGAA |
| | ACCCCGTCTCTACAAAAAATTTGAAAATTAGCTGGCCATGGTGATGCATGCC |
| | TGTAGTCCCAGGTACTCAGGAAGCTGAGGTGGGAGGATCACATGAGCCCATG |
| | AGATTGAAGCTGCAGTGAGCTGAGATGGTGCCACTGCACTCCAGCCTTGGCC |
| | ATAGAGCCAGACCTTATCTCAAAAAAAAAAAAAAAAAGTTACAATAATCTTC |
| | CCTTCAAAGCTGGAAGGCATTATTTACCTGTCTGTCCAGCAGATGGTGCTAC |
| | ATAACCAAGGGAATCTGTTGCTTGCCCTTGGTGAAGCTATTAAAGCCAATAC |
| | AGATCTTGAGAATTTCAAAAGCAAAAATCAATACTGGATTATGAGTGCTCTA |
| | GGAAAATAAAGAGATAAATTTTCAATTTACATACTTATATATAGTTATACCA |
| | TATTTGTAAATAAAAATATATAAATAATTTATCAAAATTCCTTTTTAACAGCA |
| | ACAACCACAGTAAACCCACAGGTTAAAAACTCCACAACAGTCTATATTAATC |
| | AGTCAATGCAAAGTACATTCCAATTCCAAGTTAACTGAAAATAATCAACTTA |
| | ATCATTTGGTTGGCTCTGAGCAGCCTTCACTGCTTGCTCTTGTGTCATGTTTCT |
| | TTCTGTCCTCGATTGGCTATAGACTTACAGACGGCTTTTGCAGAGGACAGTGT |
| | ACTCATGTCCATCCTTTGCATCCTTTGTGGGAGTTGGCTAGGCAGCACTCTCC |
| | CTGGGGACACTATGAACTCCTCTTCTTGAGTGCAGAGATCACGTCTTGTTCAT |
| | CTTCATGCCCAATACCTTGTTCCATAAATATGAATGGATTAGAATTCTAAACT |
| | CTTAACTCTGCCCCAAGACAGTTCTGAGAGGTAGTAAGTCATATAACACCTG |
| | AAGAGGACTGTTCTTGTCCTAATTACATTAGGTTATAAGATGACAGGTGAGG |
| | GAGCCAAACCAGGGGGCCTGGAAATTATTCATACATCTCTAGATACAGTATA |
| | CAAGTTGTGTGTATTATGTGTATTTACTCTGTAATTGATTGCTTGAGATGAAC |
| | CCCCAAACACACTCGTGTTTGGATCATTATTATCTACCCTTCTCCTTAAATAA |
| | TCTTAATTTCCTATGATGCTTGAAAGGGAAAGAGGGGCCAGGTGTGGTGGTT |
| | CACACCTGTAATCCCAGCACTTTGGGAGGCTGAGGTGGGAGCATCACCTGAG |
| | GTCTGGAGTTCAAGACCAACCTGACCAACATGGTGAAACCCTGTCTCTACTA |
| | AAAATAAAAAATCAGCTGGGCATGGTAGCACATGCCTGTAATCCCAGCTACT |
| | TGGGAGGCTGAAGTGGGAGAATCGCTTGAACCTGGGAGGGGGAGGTTGCAG |
| | TGAGCCGAGATCACTCCATTGCACTCCAGACTGGGCAACAACAGTGAAACTC |
| | CGTCTCAAAAAAAAAAAAAAAAAAAAGGCAGAGTGGGGAAGAGAGCTGCA |
| | TGAAGGAGAGATTTACTAAATAGTACTTAATCCCAAAATAATTTCTATAGGT |
| | TTGAATATGATCCCTGAAATTTATTATAGGTTCAGGTAAGTATTAATCACGGG |
| | TATTCAGAACTGTGGTTTAAAAAATGTATAGAACATGTTTCCTTCCCCTTGAA |
| | ACTTTTTATCAGCTAATTATAGGAATTATATTATACCTGCAATCATTAAAGTC |
| | CAGAATGAGACAGTACTTGGTAAAGTGCTGAAATTTATAATAAATGCATTAT |
| | AGCAATCCAGTTAAGGAGGAAGAGCCACCATTATTGAACATTTGTTAAGTGT |
| | CAGCCATTGTACTAGATAAATTTTAGTTATTATTTTTATTTAGGCACCAAAAA |
| | ATCCATGGGATAGTTGGTTATCCCCATCTTACTGAAGAGGAAACTGAAGCTC |
| | AGAAAGTTTAAGCAACTTGCACAGGTCACATAGCAAGTAAGGAGCATGGCC |
| | AGGAATCAGACCCTGATCTCCTTTGGTCTACTAAGCTTGCAAAGGATCTTCCC |
| | GCCTCCTTCCAAGACCATTCAATATTATCAGTAAATGTCCATGGCAAGGATG |
| | TAGTTCGAGTTATAGGGTTCCATTCAAGATATGATTGGTAGGTGGGAAGCAG |
| | ATATGTCTGTGTCAATCAGTATCCTGGAAGAAGGAGATGATGAACTCAAGTG |
| | GTGATTAAGGGAAGTTTAATGAAGGGACTATTTACAGAGATGTGGTGGGGTT |
| | AAGAGAACCAACAAGGGGAAGTGATGCACTCAAAAAGTTACTACCTCCAGG |
| | CTTTAGGGGATTGGGGGAGGGAGTGGCACAGTGTGAACCCAGTGTGGTTGTG |
| | AGAAAAGGGATTCCCTCAGCAGCCATGGCCAAGGTTAGAGTCTCCACTGCCA |
| | AACTGCATCCAGGTGGTGAGGGAATGGAGAATAGGGGTGGGGTAACAAACT |
| | CTGACCTCGGTATCCCCAAAGGGCAAAGGATTCCAGGTGGTACAGTTCGTAA |
| | AGATTAGCCTCAGGGCACAGAACAGGGCAGAGAAGAATGGAGAATTGATCT |
| | GGAGGAAACAAACAATGGCTTGCCCATGTTATTGCAGGGAGAGTAGGCTGG |
| | TGTGCACAGCAGGAGGGTGGGGAGCCCAGCATATAGCTGTGTTGGGGCCTGT |
| | GCAGATCAGCCTCACTGGCAGGGAGGATCTGAGCCGAGAGGTGGTGGAAGA |
| | TGAAATCGAGTAGGCATGTTGGTAGTCCTAAATATCAAGTAAACGTTCCTGA |
| | TCTTACATTGATACTCAATAGTAAGCCAATTTTGTTTCCCATAAGCCAATATT |
| | AATATTACGTATTTCTTTTATAAGCCAGAGATATAGAGAGATACCCTAGAAG |
| | AATGATAGGGGAAAGGAAGGCAAGGGTGAGAGAAGACCTTGTGTGAATTTG |
| | TCCAAAATGTTTATCCACAGGAACAATCCCTTTGTGAAGGCTGCTGGTATGT |
| | GAATGTGTGCCGGTTCCCTTGGGGCGTTCATTTGGATCTTTCTGTGTTCCAGT |
| | GACCTACGGCATGATGAGCTCCAATGTGTACTACTACACCCGGATGATGTCA |
| | CAGCTCTTCCTAGACACCCCCGTGTCCAAAACGGAGAAAACTAACTTTAAAA |
| | CTCTGTCTTCCATGGAAGACTTCTGGAAGGTATTTGCAAATAACTTTGAAAGT |
| | ACCTCTCTATCACAGAAAATTGTTCATTTGGCTTCATCATTTCAATGCATGAG |
| | TATCGACAGGACCTGCTTTGCATTTAACACTGTGTGAGACGTAAGTTATGGT |
| | GAGTTGTTAGAAGTTACTGTTCCTACTCTCAAAGGGGGTAAACTAACATTGA |
| | GAACTTTGCCTGTGCCTTGCACTGTGCTGAGTGTTTCATATCTTACCTTATTTA |
| | ATTTCTATAGTCTAACTCTATAAGGTAAGTACTAAGACTATGCCCTAGTTTGT |
| | TAATGAGGAAAATGAGATTCAGGATGTTTAAATGCGTATGGTCACATGGCTA |
| | GGGAACAAGAAAAATTGATTTTTTTCTAGCCTGACAGCTACTTCATCCTAGTT |
| | TGTAATTCATTCCATGAGTCAAGATTCAATAAATATTTATTGAGAATCTCCTA |
| | GAATGTAAGGCCAATGAAGGGCAGTGTGGTTCTTCTGTCTTGCTTCGCCTTTT |
| | GTGTTTTGTCTCTTTGTTGATGATGGCATGTATCCCCAGCTCTTAGAACAGTG |
| | CTTGATTCAAAGTAAGCACATTCTTTCAAAGGTCTGCTGTTGGTGGGGCTTGG |
| | TGGCTCACGCCTGTAATCCCAGCACTTTGGGAGGCCAAGGCAGGAGGATTGC |
| | TTTAGCCCAGGATTTTGAAACCAGCTGGGCACAACATAGTATGACTTTGTCTC |
| | TCCAAAAAAGTTAAAGAATTAGCAGGGTGTGGTGGTACACACCTGCAGTCCC |
| | AGCTACTCAGGAGGCTGAGGTGGGAGAATCACTTGAGCGATTGCTTGAGGTC |
| | AAGGCTGCAGTGAGCCATGGCCATGCTACTGCATTCCAGCTGGGGCAACAGA |
| | GTGAGACCCTTTCTCAAAAAAAATCCCCCCCAAAAAAAAACCCAAAAACAA |
| | ACAAAAAAGGTCTGCTGTTGTGAAGTTCAACCCAATCCAGCCCCTTCCCAAG |
| | TTGTCACAAATTCCAACGTAGTTAACAGTATACCAATGAGTGATACCACAGG |
| | AAAAATATTAAACTGATCTGAGGGATATGGGGCTTGGAATCTAAGAAAATTG |
| | GAAGGGAAATTGAAAAGGAAATTATTATTTCTCCTTGGGGAGATAGTTTCTA |
| | AAATTCTTACTACACCCTGGGGTCAGAGCTGTTGATTTTAAGGATAGAGACA |
| | ACTGAGTCACAGGAAACTATTCATATATAAAAGTACCTGGCATCCAAAACCA |
| | CACTTGTATAATATGAATCTTTCACCATCTGAGTAGGGCAAATCAGTCTATCT |
| | CTGTTGATCATCTGACAAGGATAGCACACTGAGAAATAGATCTGTCTTCCCT |
| | ACAGGCATAGCTAGTTGTACAAACTAACAAGAGACTTTTGTATACACATTCC |
| | ATGATGATAAATGCCAATCACTAAAGGGACGAGGAGGGATTGGAGAGTTCA |
| | CCATACAGCAAAATAGTCCAGACAGGTGAAAGGTCTATCAAATGCCAGGCT |
| | GGTAATCAAAACTGTAGCCTTTTCTCTAAACAAAGTTTAGAACCATGATTGT |
| | GTGGGACATTATTTTAATAAGGGAAAGTGCAGTTAATCATGACCCCACCTTT |
| | AGTCCAAGAACAAAAATCAGAGCTGCCACGTATTAAGTACCCACTCTGTGCC |
| | AGGTGCAGTAACTATGCAAAAGATGGGTTTTCCAGATGCAAGAACCTTGGTT |
| | CAGAGGACCCTGCTCAAGGCCTCATAGCTAACAAATGATGGGGCAAGATGCT |
| | ATCCCAAATCTCTCTGACAACAAAACTCATTCTTATCACTCTACTATTTTCAT |
| | AGAGTTGCCAAATGCTTGGTTATGCAAACGATGCAGGCAGGGGCAAGACAG |
| | CGGCTGAGCTTGGAACTTTTTCAGAGATGTTTCCTTTGCTTTTAGTTCACAGA |
| | AGGCTCCTTATTGGATGGGCTGTACTGGAAGATGCAGCCCAGCAACCAGACT |
| | GAAGCTGACAACCGAAGTTTCATCTTCTATGAGAACCTGCTGTTAGGGGTTC |
| | CACGAATACGGCAACTCCGAGTCAGAAATGGATCCTGCTCTATCCCCCAGGA |
| | CTTGAGAGATGAAATTAAAGAGTGCTATGATGTCTACTCTGTCAGTAGTGAA |
| | GATAGGGCTCCCTTTGGGCCCCGAAATGGAACCGCGTAAGTGTCTGTGACTC |
| | ATTGCCACTCGGTGATATTCATTCATTTATTCTCTGAACTCCCACCATTCATTC |
| | ATTCATTCCCTGACACCTTCACCAAGGCAAAAATAAGTTCAGTGACTCTTCA |
| | GTGCTTATATTTAAACCTTGGCCAACTTGACCTTTGACTTCTTAAGTTTTCACT |
| | ACTTCTTAGCCTTCTTTTAGTTTCTACATGCATATTTTTCAGAAGACTAAATCG |
| | TTGACCATATAACCCCTCAAAAATTAATTATCTGAGCGTTTGAAAATTTCATT |
| | TAAGATGCCCTGGGCCCTGTTTTTACAGGTGCAGTAACATCATCCACTAAGTT |
| | ATTTAACACAAGTTTTCTGGTTCAGGAACTCTTTTTATAGGTCTTGCAAACAG |
| | GTTTTTGTTCAGAATGGAGTTATTTAATGTGTAAGCTTGTGAGGCAATTTTTT |
| | GTTAGGTTTAAAGCCCATTTTGTTCAAATGTTTGAGATTTTAGGTATATATTT |
| | GTACACGTGCATATTTACAGGGGCTTTTTGTACACTTTGGTACTCCTACTTCA |
| | AACATCTTGTGTATTAAGGGAGGTCACTTACTATTTTAGAAGTATTGTAGTTA |
| | TTATAAAGAAACAAGAAGACCTCCAAGGCCGTTTCAGGGTGGGCCTTTGCGG |
| | TTGCTGTCCCTGGGTACGTCACTGGTCGGAGTCATCTTCTAAGCTTTGCTCAG |
| | CTAATTCTGTCGGTTCATCTAGGTTCTTTTCTTGGAAACTGAGTTGCCCAGAA |
| | TCCACATTTGTTACTATACAATGGGCAATCACCTTTTCAATTAGTATATTCTT |
| | CTTGTACCTTCCAGTATACACTCTATTTAATACCAGAACCCATAAGAAACAA |
| | ATTTAGTAAAAATCCAGGTTGGGCACAGTTTCTCATGCCTGTAATCCCAGCA |
| | CTTTGGAAAGCCAAGGCGGACAGATCACTTGAGGTCAGGAGTTCAAGACCA |
| | GCCTAGCCAACATGGTGAAACCCTGACTCTACTAAAAATACAAAAATTAGCT |
| | GGGTGTGGTGACATGTGCCTATAGTCACAGCTATTCGGGAGGCTGAGGCAGG |
| | AGAATTGCTTGAGCCCGAGTGGTGGGGGTTGCAGTGAGCTGAGGTCTCATCA |
| | TTGCACTCCAGCCTGGGCAACAGAGCAAGACTCCCCCTCAAAAAAAAAAAA |
| | AAACAAATTTAGTGAAAATCCAGAGCTTTAGAACAAAGGAACTAAATAGTCT |
| | CAAAGGACATTATCATCCAAGTTATGATAGTGATTTCGCTTTCTTTAAAAAAA |
| | AAATTATTACAGATAGAGTTTCTTGATGTTGCCCAGGCTGGCCTCAAACTCCT |
| | GGGCTCAAGCAGTCCTCCAGCCTCAGCCTCCCAAGTAGCTGGGACTATGAGA |
| | ATATGCCACCATGCCCAGCTTTATTTTGCTTTCTAATGTGCCTTTTTGTAGTTC |
| | CTGCAAAGCATAAGCATGCCTTCATCTGTGGTACCCTTTCCAATATTTTATTT |
| | ATCTCACATCACTAATAAGATAAATTTATACAGCCACTGCTCTGTGCCAGAC |
| | ATTATTTAAGAAGTTATTTCACGCATTATCTCATCTGCCTTCACAAAACAACT |
| | CTAAAATAGGTATCACCTCCATTTTATAGATGAAAAAACTGAGGCTCACTTG |
| | CCCAAAGTGTCACAGCTAACAAATTGGACTGAACCAAGATTTAAGCAGCCTG |
| | ACTCCAAAACCCATGTTTCGCCTACTAAACCTCTTCCATATTAATTCCTCCTC |
| | CATATTAATTGCGTCGTTAGGGTGGCTTGTCGACGCTCTCAGCTCCCCATCAG |
| | TACTCAAGCTTCCTGAGGGCAGGGATTCTATTTTGTTAACTGCTGTATTCTCA |
| | AAGCCTTGAACAATGCCTCATATGTAAAGATACTAATA (SEQ ID NO: 231) |

Alternative splice site event

| | |
|---|---|
| Canonical | TGACCTACGGCATGATGAGCTCCAATGTGTACTACTACACCCGGATGATGTC |
| exon | ACAGCTCTTCCTAGACACCCCCGTGTCCAAAACGGAGAAAACTAACTTTAAA |
| chr4 | ACTCTGTCTTCCATGGAAGACTTCTGGAAG (SEQ ID NO: 232) |
| 88036220 | |
| 88036353 | |
| Canonical | gtatttgcaaataactttgaaagtacctctctatcacagaaaattgttcatttgg |
| intron | cttcatcatttcaatgcatgagtatcgacaggacctgctttgcatttaacactgt |
| chr4 | gtgagacgtaagttatggtgagttgttagaagttactgttcctactctcaaaggg |
| 88036354 | ggtaaactaacattgagaactttgcctgtgccttgcactgtgctgagtgtttcat |
| 88038250 | atcttaccttatttaatttctatagtctaactctataaggtaagtactaagacta |
| | tgccctagtttgttaatgaggaaaatgagattcaggatgtttaaatgcgtatggt |
| | cacatggctagggaacaagaaaaattgatttttttctagcctgacagctacttca |
| | tcctagtttgtaattcattccatgagtcaagattcaataaatatttattgagaat |
| | ctcctagaatgtaaggccaatgaagggcagtgtggttcttctgtcttgcttcgcc |
| | ttttgtgttttgtctctttgttgatgatggcatgtatccccagctcttagaacag |
| | tgcttgattcaaagtaagcacattctttcaaaggtctgctgttggtggggcttgg |
| | tggctcacgcctgtaatcccagcactttgggaggccaaggcaggaggattgcttt |
| | agcccaggattttgaaaccagctgggcacaacatagtatgactttgtctctccaa |
| | aaaagttaaagaattagcagggtgtggtggtacacacctgcagtcccagctactc |
| | aggaggctgaggtgggagaatcacttgagcgattgcttgaggtcaaggctgcagt |
| | gagccatggccatgctactgcattccagctggggcaacagagtgagaccctttct |
| | caaaaaaaatcccccccaaaaaaaaacccaaaaacaaacaaaaaaggtctgctgt |
| | tgtgaagttcaacccaatccagccccttcccaagttgtcacaaattccaacgtag |
| | ttaacagtataccaatgagtgataccacaggaaaaatattaaactgatctgaggg |
| | atatggggcttggaatctaagaaaattggaagggaaattgaaaaggaaattatta |
| | tttctccttggggagatagtttctaaaattcttactacaccctggggtcagagct |
| | gttgattttaaggatagagacaactgagtcacaggaaactattcatatataaaag |
| | tacctggcatccaaaaccacacttgtataatatgaatctttcaccatctgagtag |
| | ggcaaatcagtctatctctgttgatcatctgacaaggatagcacactgagaaata |
| | gatctgtcttccctacaggcatagctagttgtacaaactaacaagagacttttgt |
| | atacacattccatgatgataaatgccaatcactaaagggacgaggagggattgga |
| | gagttcaccatacagcaaaatagtccagacaggtgaaaggtctatcaaatgccag |
| | gctggtaatcaaaactgtagccttttctctaaacaaagtttagaaccatgattgt |
| | gtgggacattattttaataagggaaagtgcagttaatcatgaccccacctttagt |
| | ccaagaacaaaaatcagagctgccacgtattaagtacccactctgtgccaggtgc |
| | agtaactatgcaaaagatgggttttccagatgcaagaaccttggttcagaggacc |
| | ctgctcaaggcctcatagctaacaaatgatggggcaagatgctatcccaaatctc |
| | tctgacaacaaaactcattcttatcactctactattttcatagagttgccaaatg |
| | cttggttatgcaaacgatgcaggcaggggcaagacagcggctgagcttggaactt |
| | tttcagagatgtttcctttgcttttag (SEQ ID NO: 233) |
| Exon | TGACCTACGGCATGATGAGCTCCAATGTGTACTACTACACCCGGATGATGTC |
| resulting | ACAGCTCTTCCTAGACACCCCCGTGTCCAAAACGGAGAAAACTAACTTTAAA |
| from | ACTCTGTCTTCCATGGAAGACTTCTGGAAGGTATTTGCAAATAACTTTGAAA |
| splicing at | GTACCTCTCTATCACAGAAAATTGTTCATTTGGCTTCATCATTTCAATGCATG |
| the alt 5′ ss | AGTATCGACAGGACCTGCTTTGCATTTAACACTGTGTGAGACGTAAGTTATG |
| chr4 | (SEQ ID NO: 234) |
| 88036220 | |
| 88036480 | |
| Intron | gtgagttgttagaagttactgttcctactctcaaagggggtaaactaacattgag |
| resulting | aactttgcctgtgccttgcactgtgctgagtgtttcatatcttaccttatttaat |
| from | ttctatagtctaactctataaggtaagtactaagactatgccctagtttgttaat |
| splicing at | gaggaaaatgagattcaggatgtttaaatgcgtatggtcacatggctagggaaca |
| the alt 5′ ss | agaaaaattgatttttttctagcctgacagctacttcatcctagtttgtaattca |
| chr4 | ttccatgagtcaagattcaataaatatttattgagaatctcctagaatgtaaggc |
| 88036481 | caatgaagggcagtgtggttcttctgtcttgcttcgccttttgtgttttgtctct |
| 88038250 | ttgttgatgatggcatgtatccccagctcttagaacagtgcttgattcaaagtaa |
| | gcacattctttcaaaggtctgctgttggtggggcttggtggctcacgcctgtaat |
| | cccagcactttgggaggccaaggcaggaggattgctttagcccaggattttgaaa |
| | ccagctgggcacaacatagtatgactttgtctctccaaaaaagttaaagaattag |
| | cagggtgtggtggtacacacctgcagtcccagctactcaggaggctgaggtggga |
| | gaatcacttgagcgattgcttgaggtcaaggctgcagtgagccatggccatgcta |
| | ctgcattccagctggggcaacagagtgagaccctttctcaaaaaaaatccccccc |
| | aaaaaaaaacccaaaaacaaacaaaaaaggtctgctgttgtgaagttcaacccaa |
| | tccagccccttcccaagttgtcacaaattccaacgtagttaacagtataccaatg |
| | agtgataccacaggaaaaatattaaactgatctgagggatatggggcttggaatc |
| | taagaaaattggaagggaaattgaaaaggaaattattatttctccttggggagat |
| | agtttctaaaattcttactacaccctggggtcagagctgttgattttaaggatag |
| | agacaactgagtcacaggaaactattcatatataaaagtacctggcatccaaaac |
| | cacacttgtataatatgaatctttcaccatctgagtagggcaaatcagtctatct |
| | ctgttgatcatctgacaaggatagcacactgagaaatagatctgtcttccctaca |
| | ggcatagctagttgtacaaactaacaagagacttttgtatacacattccatgatg |
| | ataaatgccaatcactaaagggacgaggagggattggagagttcaccatacagca |
| | aaatagtccagacaggtgaaaggtctatcaaatgccaggctggtaatcaaaactg |
| | tagccttttctctaaacaaagtttagaaccatgattgtgtgggacattattttaa |
| | taagggaaagtgcagttaatcatgaccccacctttagtccaagaacaaaaatcag |
| | agctgccacgtattaagtacccactctgtgccaggtgcagtaactatgcaaaaga |
| | tgggttttccagatgcaagaaccttggttcagaggaccctgctcaaggcctcata |
| | gctaacaaatgatggggcaagatgctatcccaaatctctctgacaacaaaactca |
| | ttcttatcactctactattttcatagagttgccaaatgcttggttatgcaaacga |
| | tgcaggcaggggcaagacagcggctgagcttggaactttttcagagatgtttcct |
| | ttgcttttag (SEQ ID NO: 235) |

NMD exon event

| | |
|---|---|
| NMD exon | GCTACCATACTCTAAAGATAGAGCCACAGATCCCAGTCAGCCATGTGATTAT |
| chr4 | GAGG (SEQ ID NO: 236) |
| 88031085 | |
| 88031140 | |
| Canonical | gtaagtagaatatttccttgcactaatgggaaagttttgaaaagatttgacctat |
| intron | ccaaatcataattaaaaggaagtgtgtatgcaccagaggggcaactgggaagtta |
| containing | ccttcttacctttgtttttaattctaatatttttatttgggcatttgtttattga |
| NMD exon | ctatcttcctatggtagaatgcaagctttataagagaagggacgtgatttgttct |
| chr4 | ctgctgtacccccatttcccaaaactgcagatggcaacagaaggctctgaaaaat |
| 88019572 | atataagaaagaatttttctaattgtgactaaattgtgaccaaatgctaagtgac |
| 88036219 | tgtggacttgcgtttaacacaggacgggagaggcaaagagttcaattccaattta |
| | gaatttggtcaagttctcttctgcactctggtaaacattaattaaaaatcagcat |
| | tatctgaccagccagttcatcgtcagtggtggtgattttcactatgagatacgcg |
| | tggcaacttgccagacaccaagaaaccaagttagaggatttttgtattagattcc |
| | ttaacaatgaatacagtatcaccattattacagtatcatcattattgtcatacta |
| | ttattatatcagttaacataaagtctgcataagaattgtttccagaaaaatgact |
| | ttccaaatttaactttcaggaaatacaaataatgctactaatattgcttttattg |
| | gcgtatacatgtaatatccccttcttttggatttggatatgttgtgtcattgcct |
| | cattttaattcattatttcttctcaatctttaataattgctggacttttactcca |
| | caagaaacttgctataggcccatctctttcgtcttctttccttctttcagttcgt |
| | cttcccatcctctggtagggggaggggagggatgcctgagcgagagactagctgt |
| | aggaaccatttgtctcaaagtccagaaagccacaggtgatggatttgtcctctga |
| | atcaaagggcgttcgatgatggatttctgtcatgtctcatctaaagtcttcacga |
| | gaacagatgaggaagcagttttatgaccccagagcctcctaccaaactcctctga |
| | gaaaaggtttccttttttttttttttttttaaattagagacagagtcttgatctg |
| | tttcacaggccggagtgcagtggcacaatcatagctcactgcagcctcgagctcc |
| | taggcttcagtgatcctcccacctcagtcacccacgtagctgggactacagctgc |
| | acaccaccatgcccagctaacttttaaaacatttttgtagaggtggggtctcact |
| | ttgctgcctatactggtctccagctcctagcttcaagtgatcctcctgccttgga |
| | agttctgggattataggcatgagccactgcacccagcctggatgtgatattttta |
| | tgttttaaattgttagagtttagaaacttgagattgagtttgctgcctgcattaa |
| | aatgatgcttaaacattaaactgcagtggccttaaatattaacaagttgattaga |
| | attactaagttcttttcaagctttacatatacagacaaatttcttatgcaaaata |
| | gaaggtaacccctgtacgtaagtctagaatttcagcagtccccaaaactgactga |
| | gcattagaatcactctgattttaaaatacatatgtggttttccgagatctactaa |
| | cagagcctccataatgtagcctagagaaacgagttttcagatgtagtgataaatt |
| | tggaaggtaagtcagagaagtaagctgaagacagagttttaggaaatatgcctaa |
| | agtcacataatgaattggttttcttgtttatttgagaatattgtcgctttttgtt |
| | ctttttcatgcaaatcacattttatttcttatgtgagtagctatatatttaaaaa |
| | ttttgtttttggaatattgtagaatctctacttaagaaagtatcttagcagtcat |
| | atggtctgacctcactgaatgctaaattctctttaaaacatccgtctcagatggt |
| | tactcatactgcctctgattgaacaagagtcccaggttaagggacttacttcttt |
| | gaaatcgttcatttcatttttctacagctatgttagaaagttcgtccttagcagt |
| | gaagccagagtctatctcttataacttctacccagttgcaccctccaagcctacc |
| | tataccaagtatcttttttccacgttgcttttccaattcagctcttcgcaagttt |
| | ggagacaaatacctaatctcctctaagccttctccaggttaagcctttccagttc |
| | atccagctgttgattatgtgattggagacacaagtttgagtaactcccatgatgg |
| | aaagtccctctggtgaatgttctgttcatcagagtccctaagaaagcacatgagc |
| | ctcaccatggtgagtggggccatgagattcctaaaccagacactacactgtggct |
| | tgtgcaacctaccatggccagactcatgaggctgtttcatcatataaataattcc |
| | ttagtctcttcaccaaaaactgtgaagcactgtgtcccccagctgtatgtgagct |
| | agcctgggaccatagtggaggactcctctttcaaccctgttaaatttcatcttgt |
| | taggatctgcccatttttccattctgttgaaattatcttggaactggattcattc |
| | atctcacatcagctactccccgaagcctcacagtatcagcagagttattatctct |
| | atccatatccttgtaaattattaaactaaaaaagattggtctaagaacatcagtc |
| | cattaatcaaggctctttggttggagttcacttcattatattatcatccagctca |
| | gagtgttcataaacaagatgataggaaagacttttccaaatgcctgtctgagtaa |
| | ttcccccattcctgtgatctgtgagctgttgaccagattaaaaaggaagtgagaa |
| | taaccaggcaagatttactcctagcaaaaccttactggcttctagtgactgagtc |
| | ctttccctattgcttaccagctatctttttaattactagttttaaaatcttgcca |
| | gcaatgtcaatatcaaatgaccaagaatatcaaactcattactctgtatgtaaat |
| | gagatagtgtacttacccctatggcttaagtattaggctgttcttgcattgccct |
| | aaatacctgagactgggtaatttataaaaaaagaggtttggccaggcactgtgga |
| | tcaggcctgtaatctcagcactttgtcaggctgaagcaggtgtaatggtgagcca |
| | agagttcaaacttagcctggacaacaaggtgaaaccccctctctgcaaaaaatac |
| | aaaaattatctgggcatggtggcatgcacctgtagtcccagccacccaggaggct |
| | gagggggaaaattgtttgaagctgggagtcagtgattgcagtgagccatgattgc |
| | aacactgcactccatccagcctgggcgacagagcaagaccctgtctcaaaaaata |
| | aataaatgaataaataaataaaataaataaataaatagaaaagaaaaagaaagaa |
| | aagagatttatttgcctcatggttctgcaggctgtaagggaagcatagctccagc |
| | atctgcttctggggaggcctcaggaagctgttactcatggcagaaggtgaagcag |
| | gagcttgcacatcatgtggcaaaagcaggagcaagagagagagaatggggcaggg |
| | aagaagccccacacttttaaatgaccagatcgcatgagaaatcactcgttacctc |
| | aaggacagtaccaagaggatggtactaaattcctgagaaatccacccccatgatc |
| | tgatcacctcgtaccaggccccgccttcagcattggggattatgtttcaacatga |
| | gatttggatggggacaacatccaaactatatcaccttgcatagtaggtagggttt |
| | taaaaagcagtttggcacagtaagaaaagtacagattttttttgcatcagacaga |
| | cctgagttaaaatcccagcttcactgctaacatgctaggtaaatgtgggcaagtt |
| | aattaacatttctaagcctttgtttcctcactggtaaaacaagtatttggaaata |
| | tcattgtgaagattagaaataatacatgaaaagatcctaggatgctgtctgtcat |
| | acagtagtagtagtaagaagttattcttgccaaagattgttgagaatggcagaat |
| | tatctcagttctaagagctatagtttctaattatttgagcctagactcagattca |
| | tttggagcagctaactgctcaccaagagcttattttccatcttaccaatgaggtt |
| | atgtgccctgtgtttttaaaatcagtctacttaaccaagagaacagaaatgacat |
| | gagaattaagtaatctcactttctctgttatttaggatttattcctactcaaaac |
| | ctgagagttgctatgaattcaccattaaagcacttattaatatacatgggttact |
| | gttataaatagcaatagtattgctattgtgtgagttaggtgttgaagttcaagaa |
| | aggaataaagaatatttagaagatctttgaaaacagtgtctgggtacggtggctc |
| | atgcctgtaatctcagcactttgggaggccgaggcaggcagatcacttgaggtca |
| | cgagttcaagaccagcctgggcaacttggcgagacctcgtctctacaagatatac |
| | aaaaattagccgggtatgttggcatgcacctgtaatcccagctacttaggaggct |
| | gaagcacaagaatcacttgaacctgggaagcagaggttgcagtgagccaagattg |
| | taccactgcactccagcctgggcaatagagcaacactctgtctcgaaaaaaaaaa |
| | aaaaaaaaaaaaaaagaaagaaagaaggaaggaaggaaagaaaaaaaaaggaaaa |
| | caatgaaggaaggtatttggtgaatctataataataaaaatgtatttgtcatttc |
| | ctttttctgtgctctcattctataaaattgagtaaaaaatctatatatagtttaa |
| | acacattaatagaaatcacaaaagttagctgagtcaacattgtagaaacataata |
| | tttctgtatgtcaaagaaatagacaacattaaaaagcagaaagcaattacaaaga |
| | atgattataagaaacatcacaaagggttaatattttaacacatttgaaactcaaa |
| | aatcactgagaaaagcagtagacttccataaatattttatagagtagaaaaaata |
| | ggccaagcacagtggctcatgcctgtaattccagcactttgggaggccgaggagg |
| | gtggatcacgaggtcaggagttcaagaccagcccggccaagatggtgaaacccca |
| | tctctactaaaaatacaaaaactagccaggcgtggtggcaggtgcctgtaatccc |
| | agctacttgggaggctgaggcagggaattgcttaaacccttaaacccgggaggtg |
| | gaggttgcagtgagccaagttcgcaccactgcattccagcctgggcgacagaacg |
| | agactctgtctcagaaaaagaaaagaaaagaatagaaaaagaatccatgggcagg |
| | cacagtggctcatgcttataatcccagtactctaggaagccaaggtgagaggatc |
| | aattgaggccaggagttcaaggccagcctgggcaacatagcaagactttgtctct |
| | attaaaaattttaaaattagccaggcatggtgacgcacacctgtagtcccaatta |
| | cttgggagcctgaggcaggagaactgcttgaggctgcagtgagctatgattagac |
| | cactgcactccagcctgagctacacagtgagaccttgtgtcaaaaaagtaaaaaa |
| | ataaaaattagccaggcatggtggcacatgcctgtagtcccagctactcaggagg |
| | ctgaggcaagaggatgacttgagtctggaagatggagactgcagtgagctgtggt |
| | catgccactgcactccagcctgggtgacagagcaagaccctgtctcaaaaaaaaa |
| | aaaaagaaaagaaaagaaaaataaataaaatttattcaaatacaaaagtgatgtg |
| | gtttgactctgtgttgccacccagatctcatctccaattgtaatccccgtgtatt |
| | gacggaggttcctggtaggagatgattggatcatggggatggtttcccctctgct |
| | gttctcatgatagtgagtgagttctcatgaaatctggttgtttggtaggtgtctg |
| | tcacttaccccttctttttctctctcctgctgccttgtgaagaaggtacttcctt |
| | ctcctttgccttccaccatgattataagtttcctgaggccttcccagccatttgg |
| | aactgtaagtcaattaaacctctttcctttataaattaccgagtctcaggcagtt |
| | ttttatagaagtgtgaaaatggtctaatacagagacttggtaccaggagtggggt |
| | actgctataaaaaataacctgaagatatggaagcgactctggaactgggtaacag |
| | gcagcaattggaacagtttggagggctcagaagaagacaggaagatgtgggaaag |
| | tttggaatttcctagagacttgttgaatggctttgaccaatacactgatagtgat |
| | atggacaatgaagtccaggctgagatggtctcaggtggagatgaggaacttattg |
| | ggaactggagtaaacgtcactcttacatgttttagcgaagagactggcagcattt |
| | ttcccctgccctagagatctgtggaactttgaacttgagagacatgatttagagt |
| | atctggcagaagatatttctaagcaccaaagcattcgagaggtgacctggctttt |
| | cctgaaagcatacagttatatgtgctcacaaagagatggtttgaaattggaactt |
| | atgtttaaaggggaagcagagtgcaacaaaagtttagggagtttgcagcctgacc |
| | atgtggtagaaaagaaaaacccattttctggggagaaattcaagctggctggaga |
| | aatttgcataagtaacgaggagctgaatgtgagttgccaagacaatggggtaaat |
| | gtctccagggcgtttcagaaaatcttcagggcagaccctcacaacacaagcctgg |
| | aggcctagaagggaaaaatggtgtgagccaggcccaggcccaggccccagctgtt |
| | ctgtgcagccttgggacatggcaccctgtgttccagccactccagctccagctgt |
| | ggttaaaaggagccaaggtacagctggaccattgcttcagagggtacaaatccca |
| | agcattagcagcttccatgtggtgttgggtctttgggtgcacagaagacaaaagt |
| | tgagctttggaagccgctgcctagatttcagaggatgtatggaaacacctcgatg |
| | tccaggcagaagtctgctgcaggggcagagccttatggagaacctctgctagggc |
| | aatgcaggggggaaatgtggggttggagctcccacacagagtccccactggggca |
| | ctgcctcatggagctgtgagaaaaggaccaccatcctccagactccagaatggta |
| | gatccaccaacagattgcactctgcgcttagaaaagctgcaggcactcaatgcca |
| | gcctgtgaaagcagctgcaggggctgtaccctgcagagccacagaggtggagctg |
| | tccaaggccatgggagcccaccccttgcattagcatggagacaggggatcaaagg |
| | agattttggagatctaagatttaatgaatgccctgtcgagtttcagacttgaatg |
| | gggcctgtgacccctttgttttggccaatttctcctatttggaatgggaacatat |
| | acccaatgcctgtacccccattgtatcttggaagtaactaacttgcttttgattt |
| | tacagactcaggcagaagggacttgccttgtctcagatgagactttggacttgaa |
| | cttttgagttaatgttggaacgaattaagacattggggttctgttgggaaggcgt |
| | atttggttttgaaatgtgagaaggacatgagattttggaggggccaggggtagaa |
| | tgatatggtttgactctgtgtctccacccaaatctcatctccaattgtaatcccc |
| | atgtgtcaagggagggacctgatgggaggtgactgaatcataggggcagtttccc |
| | ccatgctgtttgcatgatagtgagggagttctcatgagatctggttttttggtaa |
| | gtgtctgggcttcccccttttccctctctctcctactgccttgtgaagaaggtac |
| | ttgcttctcctttgccttctgccatgattgtaagtttcctgaggtctccccagcc |
| | attcagaactgtgagtcaattaaacctcttcctgcctattctcaggcagttcttt |
| | atagcagtatgaaaatggactactacagaaagtgtgtaactttaaactcagtagt |
| | atccaaagaagtaatgaaaatggagaaacgaacaacaaaatcatagtacaatatg |
| | gtgtatgtactaggacaggaagagcccttttaagaagagatctatgtatttccat |
| | ttgtttatctctgaaagaaagcaactttgccttgtattctgaaaaagaaaggaat |
| | attttattttacttgtaaaaatcttacaaggatgctagtctaaatatagttttcc |
| | taatttgccagagaatccatgaagatcgagttgataacaagatcagtgaagtaaa |
| | ggtcagtgagttaatctcacagcagctgcaggctaattccatttccagtgaaaaa |
| | cgtcttgattgctcaccacatatcttttcaccacaaacagtttcagtcttaagat |
| | cacatgttgcaatccatgagaagtaactattaagccttcaactatgactggaggg |
| | ctcctcgccctttctgataaattgactggacaaaaactcaattttaaaatgacaa |
| | gaaatagaagatgtataaatgtactttaaatgtgaccaaaatgggttgtgaaaac |
| | acaagacacaatatccaaaaatgctggcaacacagtacactgtagagtattggtt |
| | gtttatttacccttgctattgtgtggctgagcttactgccactgcccagcattgc |
| | aagggcatcaaactgcctatcactagcctaggaaaagatcaaaattcaaaattct |
| | aagtacagtttctactgaatgcttatcacttttgcaccattttaaagtaaaaaaa |
| | tcagtaagttgaaccatcatatatccaagattgtctgtatataaatattatacat |
| | ctttctctcacttttaaaacaaaataatactagccaatactaccattctcaaaag |
| | cacttgtgtcaacagcctttaccccttaaagattttcctcacaattttaaaattg |
| | ttacttactattttctttgaaatgttgaccaaacctggattaaaagatttggggg |
| | ttttagtgactgtatttcacaaactctcttattgattctgcagcctcacttctgc |
| | ctcctaaaaagccctcaccaaggtcacgggggatggctcttttcagcctcttcct |
| | ggcatttggtccagtggcatttggcattctaggacttccctctttgtctttgata |
| | actccctctcttcctgtgttcctccttgctgtgttcacttgcttcgctttcttct |
| | ttctgaagcatgtttacacagtgttttctctgattgggcctgtgacgttctttag |
| | gtcatcttttccacaaataatgcttcaactagtacttgcgtgccagtgactccac |
| | gtcccactcatgagctctgaacctagtaccagcttctgctggacatctacaatgg |
| | gatctctcacaggcctctctcattggtaacatgccccagcctgaactcatctccc |
| | acccatctatccagccatgctctctagttcacctgaacacttgggtgtcatccta |
| | gatgctttcccttcccagtcttctgtgatcattctgcctcatcagaggctctcta |
| | atctgtcttctttcctatatcgctcttgtccctattttaatcctaatcatctatt |
| | tcctgacttattcattccttaagttggtcagtaatttaattaaaaacagatttag |
| | gccctgaccttaaatgtgataagtgatatgaaaggagatgactggggaaaaggat |
| | ttccctcaaggaaggcctctgtgaagcctgaagcaagaatgaaaacgagtcagac |
| | gaagagagaattgtatgaatgaaggctctgaggcaggaaaacactcagatcattc |
| | cagaatcacttagaagccaagtgaagccagttcctggagagcagatcatcaaatg |
| | aagatggaaaggtgaccaggggccagacctgtagttttggtgggccttggtgagg |
| | gatttacagtaggacaccccatggtttaagtatgaaagtgacaagattcctttaa |
| | gttttaagaggcctcgaaatatgaaccacagattagatggaagctactctccctg |
| | tgtctggactttttagaatttccaagagctgctgtttctggaaccagattaatac |
| | aagtcagtcttccatttatttatttatgtatttatttgagacagggtctcactct |
| | gtcacccaggctggggtgcagtggcatgaacacagctcactgcagcttgggggct |
| | caagagatcctcctgcctcagcctcccatgtagttgggaccacaggcacctacca |
| | cccagctaattttatttgttgtagaaatgaggtctcattttgctgcccaggctgt |
| | tcttgaactcttgggctcaagcgatcctcctgcaacatcttcccaaagtgctggg |
| | atcactcttccatttaacatgctatctcaacgtcaagataaactttaaaatcttt |
| | agataataggctggcattttacttaaacgatctttacttcttcagaactgccatt |
| | ccctataaatatctggttcttcaaccacatcaaaccacttgtgatctcaaaaagc |
| | ctcagcgtacactgtcctttctgtcattctaattcctcctcatccttcaaaatca |
| | actcaaggaccagatccagggagaagcttagtggtgcccacccgaaccggccccc |
| | tccttcgagttgtgctgccattcgggcccacctcttcacacagggttgtcagacc |
| | agaccagctcatgcgttcaccgcccttgcagggatgggatgcagctgtgcacctc |
| | tcagtgccgacacctggagagtctcacgaaatgttgacaacatggcctgtttcca |
| | tttcttgttcactaggacttctcatttactaacacacagaatttcctgtagtatg |
| | tccacttaatcagttcaagcctaataattccttgatttgggtatagtgctttgca |
| | tttatatactgatggtccccaacttacgatggttcgatttatgatttttcaactt |
| | catggtgatgtgaaagtgatacacattctatagaaaccacactttcaattttgaa |
| | ttttggtctttttccaggctaccatactctaaagatagagccacagatcccagtc |
| | agccatgtgattatgagggtaagcgaccaatactctacagtgtattgtattgcca |
| | gatggttttgcccaactagcctaatgtaagtattctaaacatgtttaaggtaggc |
| | caggctaagctgtgttgctcattcagtaggttaggtatattaaatgcattttcaa |
| | cttatgatattttcaatttacaatgagtttatcaggatgtaactctactataagt |
| | caaggatcatcttgtatagcacttttaatttatagagtcccttcaaatgtttgtt |
| | tgtattttatttccactgcatccctgtgaggataccataagttatacagctaaca |
| | aaacagttagttttcctgtgcaaagtgatggcttcatcttgtggcagattacctg |
| | gaatactgtggccaaggcatcttagttctactgtctttatatatctagtacagtt |
| | atatttttatggcagctctgatttcttctttggcccaagggttattaagagaggg |
| | aaaaaatttaatttcttaacagatatatatatctatgtcaagtcatatatttaat |
| | tcaaacccttaatattcctaggtaatttttgtctactttctctgtcaaagattga |
| | aagatacagggttttaagtttccaactgtaattgtagttttggtaattgttttat |
| | tactaacaggtattgctctgtatattttgatgttctgttattcaatacataagaa |
| | ttcattacagttgaatcatcatgtatagtataatttaccaatgtaaaataacctt |
| | tttatcaaactgagtattcaccttataatgtcctctgtcagaggattataatacc |
| | acttaaaaccttttaaaaaatattttgttcctgataattttagctttgagtggtt |
| | ttcttataacaattctagagatgcgtttttattttttaaccaagatttaaagctt |
| | tgtgatttagtaagagtctaaaccattcacagttcatgcttttttgtcaaatttc |
| | tattatttagtattttcctctcttttatattttcctgttattttccatttctatt |
| | cttttgttagactggaaattttttctttgctttcttttatcctagtgatttgaaa |
| | tttatgtaatatatactattctacaataccctttatttgttttcaatatttgaac |
| | ctatatttttcaacattattaagaataaaatagtatttgttgctattttgaaatg |
| | atagaccatgtttttaaggcaggttggtggttgttaaggcaccagtatcggccag |
| | gcacgatggctcacacctgtaatctcagcactttcggaggccgaggtgggcagat |
| | cgtttgagcccagcactttggccgatactgtggtttactgtattttgttctagtt |
| | tattttatggaaaatgggaattcagtggttaagacaaggattaaatagcagaaga |
| | aagatgtgtacatatgtacagatgtatgtgtcctttatatgttttttagtactct |
| | tgtctcctttctggtcctcatttaaggttatctatttcatgcagtaaatttttct |
| | tcacaattcatttcatttagagagtgaatgctaccttccaagtgggctttctcca |
| | gttttcctttcagggacttaaaggagaagtgatgttaacagttttatatttccat |
| | tgcattttacagtgtgcagatgtcttcacatatatttccccatttgagctttaca |
| | aaagcccttagtattattctcattgtctagattccaaaatcaggcttagaggagt |
| | taaatagttgtccaggatctcaagatgcaagacccacaatcatgaacagaggcag |
| | atgttcaggatggaggcaagctgaaactcaaaaccaaatcattatgactccaaat |
| | tcaggagtcttttagctgccacctgcatgggctcttggtgtagctgaccaccaga |
| | gtttgtagagctgtcattcaggtgtgccatggactttcctgggacctggcacagg |
| | agaaggactgagttaatgtttgctgattaaatatctgttacaggctgggcgcggt |
| | ggctcacgaccgtaatcccagcactttgggaggccgagcagggaggatcacttga |
| | gctcacaagtttgagaccagcctgggcagcatggcgaaaccccgtctctacaaaa |
| | aatttgaaaattagctggccatggtgatgcatgcctgtagtcccaggtactcagg |
| | aagctgaggtgggaggatcacatgagcccatgagattgaagctgcagtgagctga |
| | gatggtgccactgcactccagccttggccatagagccagaccttatctcaaaaaa |
| | aaaaaaaaaaagttacaataatcttcccttcaaagctggaaggcattatttacct |
| | gtctgtccagcagatggtgctacataaccaagggaatctgttgcttgcccttggt |
| | gaagctattaaagccaatacagatcttgagaatttcaaaagcaaaaatcaatact |
| | ggattatgagtgctctaggaaaataaagagataaattttcaatttacatacttat |
| | atatagttataccatatttgtaaataaaaatatataaataatttatcaaaattcc |
| | tttttaacagcaacaaccacagtaaacccacaggttaaaaactccacaacagtct |
| | atattaatcagtcaatgcaaagtacattccaattccaagttaactgaaaataatc |
| | aacttaatcatttggttggctctgagcagccttcactgcttgctcttgtgtcatg |
| | tttctttctgtcctcgattggctatagacttacagacggcttttgcagaggacag |
| | tgtactcatgtccatcctttgcatcctttgtgggagttggctaggcagcactctc |
| | cctggggacactatgaactcctcttcttgagtgcagagatcacgtcttgttcatc |
| | ttcatgcccaataccttgttccataaatatgaatggattagaattctaaactctt |
| | aactctgccccaagacagttctgagaggtagtaagtcatataacacctgaagagg |
| | actgttcttgtcctaattacattaggttataagatgacaggtgagggagccaaac |
| | cagggggcctggaaattattcatacatctctagatacagtatacaagttgtgtgt |
| | attatgtgtatttactctgtaattgattgcttgagatgaacccccaaacacactc |
| | gtgtttggatcattattatctacccttctccttaaataatcttaatttcctatga |
| | tgcttgaaagggaaagaggggccaggtgtggtggttcacacctgtaatcccagca |
| | ctttgggaggctgaggtgggagcatcacctgaggtctggagttcaagaccaacct |
| | gaccaacatggtgaaaccctgtctctactaaaaataaaaaatcagctgggcatgg |
| | tagcacatgcctgtaatcccagctacttgggaggctgaagtgggagaatcgcttg |
| | aacctgggagggggaggttgcagtgagccgagatcactccattgcactccagact |
| | gggcaacaacagtgaaactccgtctcaaaaaaaaaaaaaaaaaaaaggcagagtg |
| | gggaagagagctgcatgaaggagagatttactaaatagtacttaatcccaaaata |
| | atttctataggtttgaatatgatccctgaaatttattataggttcaggtaagtat |
| | taatcacgggtattcagaactgtggtttaaaaaatgtatagaacatgtttccttc |
| | cccttgaaactttttatcagctaattataggaattatattatacctgcaatcatt |
| | aaagtccagaatgagacagtacttggtaaagtgctgaaatttataataaatgcat |
| | tatagcaatccagttaaggaggaagagccaccattattgaacatttgttaagtgt |
| | cagccattgtactagataaattttagttattatttttatttaggcaccaaaaaat |
| | ccatgggatagttggttatccccatcttactgaagaggaaactgaagctcagaaa |
| | gtttaagcaacttgcacaggtcacatagcaagtaaggagcatggccaggaatcag |
| | accctgatctcctttggtctactaagcttgcaaaggatcttcccgcctccttcca |
| | agaccattcaatattatcagtaaatgtccatggcaaggatgtagttcgagttata |
| | gggttccattcaagatatgattggtaggtgggaagcagatatgtctgtgtcaatc |
| | agtatcctggaagaaggagatgatgaactcaagtggtgattaagggaagtttaat |
| | gaagggactatttacagagatgtggtggggttaagagaaccaacaaggggaagtg |
| | atgcactcaaaaagttactacctccaggctttaggggattgggggagggagtggc |
| | acagtgtgaacccagtgtggttgtgagaaaagggattccctcagcagccatggcc |
| | aaggttagagtctccactgccaaactgcatccaggtggtgagggaatggagaata |
| | ggggggggtaacaaactctgacctcggtatccccaaagggcaaaggattccaggt |
| | ggtacagttcgtaaagattagcctcagggcacagaacagggcagagaagaatgga |
| | gaattgatctggaggaaacaaacaatggcttgcccatgttattgcagggagagta |
| | ggctggtgtgcacagcaggagggtggggagcccagcatatagctgtgttggggcc |
| | tgtgcagatcagcctcactggcagggaggatctgagccgagaggtggtggaagat |
| | gaaatcgagtaggcatgttggtagtcctaaatatcaagtaaacgttcctgatctt |
| | acattgatactcaatagtaagccaattttgtttcccataagccaatattaatatt |
| | acgtatttcttttataagccagagatatagagagataccctagaagaatgatagg |
| | ggaaaggaaggcaagggtgagagaagaccttgtgtgaatttgtccaaaatgttta |
| | tccacaggaacaatccctttgtgaaggctgctggtatgtgaatgtgtgccggttc |
| | ccttggggcgttcatttggatctttctgtgttccag |
| | (SEQ ID NO: 237) |

TABLE 3
Sequences of exemplary PKD2 pre-mRNA transcripts and mRNA
transcript sequences
PKD2

| | | |
|---|---|---|
| pre- | premrna | AGGCGGCGGCGGGCGCCGGGAAGAAAGGAACATGGCTCCTGAG |
| mRNA | ENST0000 | GCGCACAGCGCCGAGCGCGGCGCCGCGCACCCGCGCGCCGGAC |
| transcript | 0237596.7 | GCCAGTGACCGCGATGGTGAACTCCAGTCGCGTGCAGCCTCAGC |
| | | AGCCCGGGGACGCCAAGCGGCCGCCCGCGCCCCGCGCGCCGGA |
| | | CCCGGGCCGGCTGATGGCTGGCTGCGCGGCCGTGGGCGCCAGCC |
| | | TCGCCGCCCCGGGCGGCCTCTGCGAGCAGCGGGGCCTGGAGATC |
| | | GAGATGCAGCGCATCCGGCAGGCGGCCGCGCGGGACCCCCCGG |
| | | CCGGAGCCGCGGCCTCCCCTTCTCCTCCGCTCTCGTCGTGCTCCC |
| | | GGCAGGCGTGGAGCCGCGATAACCCCGGCTTCGAGGCCGAGGA |
| | | GGAGGAGGAGGAGGTGGAAGGGGAAGAAGGCGGAATGGTGGT |
| | | GGAGATGGACGTAGAGTGGCGCCCGGGCAGCCGGAGGTCGGCC |
| | | GCCTCCTCGGCCGTGAGCTCCGTGGGCGCGCGGAGCCGGGGGCT |
| | | TGGGGGCTACCACGGCGCGGGCCACCCGAGCGGGAGGCGGCGC |
| | | CGGCGAGAGGACCAGGGCCCGCCGTGCCCCAGCCCAGTCGGCG |
| | | GCGGGGACCCGCTGCATCGCCACCTCCCCCTGGAAGGGCAGCCG |
| | | CCCCGAGTGGCCTGGGCGGAGAGGCTGGTTCGCGGGCTGCGAGgt |
| | | aagagcgcgcgacccgcagcggcagatgcacgaaccagaacggcc |
| | | ggcgccggccggggccatcgcccgctgcggcagctccccgggctc |
| | | catctcgcatcccctctgcgttccgcctcccttggaagcgcattc |
| | | cccacctccgctagtgctgccctatttccggtacccagcgcggaa |
| | | ttccactgctcttttgttggtgcatatttattggatacctccttc |
| | | ttcaggatatgtcaccatagtcttttttactgaaaattagtgaaa |
| | | gcctaattagagtgaaagagtacatctgggttttgtttttttttt |
| | | tcttgtagaggaaaaaatgaacattacttgtgtaactgatggtag |
| | | ttgcaactgcatatttgccaatgtcacaaaatctaaaggaaaatg |
| | | ttatagtcacccgtggtttccttcttgcctggacactccattgtc |
| | | ccgggctgaaaagggtagcagtacagtgcatataatgtcaagttg |
| | | tgggaggagtgtggcagattgtcattggtgcatttttttggtgat |
| | | gtgtgtggttgttttgaggagtgggagctgttaagaacaccacaa |
| | | atagaataaaataatatcgtgaagttatttggccgtttctaattc |
| | | tagacatttttctaaaaacagttgcaaaggaaagattacattgtt |
| | | ttaaaaaaatttgaagtatgtttttaaataactaaattaatgttc |
| | | tttgaaattccaccaaaatgatgaagtcaccagatagcagctgat |
| | | aaatgtgtctgagcccagtgcgcccagctctacaaaaggcagaag |
| | | aggaattttcaaatttgccagtgcccagtaagaggacatgaattt |
| | | tctagtaccagaggaaattctctttttacaaattttgtcagaggt |
| | | atccttgggaaagtatttcatttgcttttaccctccaaattattt |
| | | taatctatatttttaagagtttccattctcagttgagtttttctt |
| | | gttcttctccttctgtcagttttgaaagtcttgcacaaaaacaat |
| | | ccaggtgttgttacagcagtgtgattaaaaccaggtcaggcctac |
| | | actgaaatcccagttccaccactcattagctgtacgaccttgagc |
| | | aagttacttatcctctctgatacccaatttttggatttttttttt |
| | | ttttaaagagatgatcataacgcttaggacttgactctttgggag |
| | | aattaagtgagttaagacataaaatgtgcagcatgtatttgtcat |
| | | gttattagcgcttcagaaatataaatgtaaacattttggtacttc |
| | | gtttatggagattcttatactagttaattttattaaaagcatgat |
| | | ggggaacagaagatccttttcggataacctgtgtgagtaaattaa |
| | | taaaacactaacactttttaagattcaaaactggattaattatgt |
| | | tattttacaccatttaaaatgtgcatttaaaaaatattcactgaa |
| | | gtgagagagaagttccttttaagttaataaataatggtaagttgc |
| | | atgatccttttaactcagtttaagcatttgataacacccctaacc |
| | | ttgtttgaataaactcaataacagaatatagagaaataaaatata |
| | | tatttcatatgagtatgtatgtaaatttatttcttttaaaggaaa |
| | | atttcaggaaacaaaatgaataagctcatagtcataaaacctcta |
| | | gccaaagtgtgtcaatggtctgatttgtaggagagcagactcagg |
| | | aatcgggaattttttttttttttttaagagtcggggtttcactct |
| | | gtcactgaggctggagtgcggggaagcaaacgtagctcactgcgg |
| | | cctcaaactcctgggctcaagctgccctcccacctcagcttccca |
| | | agtagttgggattacaggtgcaagctgctgctcctagctgaaaag |
| | | ttttaattatataaaatgtttaaataaattgttattcctttcttt |
| | | tatgaagaaatataattgatattcttgtcacttattaaataagca |
| | | acattttaaaatgtttagcacctactgtgaagatagcactgtgct |
| | | agctgctatgaataaactagaatagcatatcactgttttcctatg |
| | | gacaaaaacccagaaatgtaaaataaatagccatgaatgggttga |
| | | gattccccctgccccctttggatactgatgacagaaatcttatat |
| | | gcactatgtaagcagtgccctaggatcagaacagaaagtaacttg |
| | | caatttttaaaggtgggagaaatgactgaagtttggagtggtcag |
| | | aggtttccctggaagtctcttgcctttgtgactttgtatagctcc |
| | | ctcaaactacctgagcctgagctcctcatttataaaatggaagaa |
| | | ttgaacctagcttccaaggtcctttctagcttcaccatgctctgg |
| | | tctgttgtttaatgacatgaatccaagatggacaacaaatggtcg |
| | | attttgctccttctacagtaaatagatctatcttcttagcagaag |
| | | taaaatagtaaagaaagaagacatgtttgaggcctgttgaggcct |
| | | gtttgctgtatgcattgtatctaatcaaggaattgagaatgtagc |
| | | cctaaatattaggaaggagttgaaagtttctgagcaggaacagaa |
| | | catgctgaaaggaaattgtgtcagcagcatggtgtacaggagagc |
| | | ttggcagagagaaaggaggcaagaagacctgtgggaggcagccag |
| | | taaggaggtgaagagggcctggaccaaaggaagccaaggatgacc |
| | | gaaagatttaaagatgaagcccagatttaaaagtatcctcaagta |
| | | tttgtttattcttttcttagcaaattctttttagtacaaagataa |
| | | aatatggcactctgagtcataaaattttctgaatttcagaagttg |
| | | aatgtttttctgaatgtatcaacctgttaaagtcagttcctgttt |
| | | gttattttaggcgtatattcttgggcttttttttttttttcctag |
| | | agaaactatgaagtactagctgtgcaagtcaggggggctaagctg |
| | | ctaaaacagatacctccctccccttcaataccctgaagaacaaga |
| | | agttaattttttgctcgtgtaacattttagagagggtattccagg |
| | | ttgaatacctggaataatgaatgaaataataagtcgactcttatt |
| | | tctcctaaaataattggatcttgacattgaaattggtgtgctgat |
| | | tgtatttaataagtgacctccaatgcagttatttcattttgccat |
| | | actttatgtaatttttattttttctgccttccctttcgatgtcta |
| | | aagggaagcataattagttattggaaaggttattgagacttaaaa |
| | | aaagatttgaacatggctttggttgatgctaccacacagaaactg |
| | | caaacatccaactcaccgctcttagttgctgttcatgatagtcta |
| | | tgcacagtgactgatgaatttagcctgtcttgaggcttcagaact |
| | | tagtcttatgtcatcacgagcagagctttatcctaaattacaggt |
| | | tttcaatgggggggggggggaggggcagttttgcccctcagggga |
| | | catttggcaatctctggagacatttttgattgtcatacctggggg |
| | | gatgcccctggcatctcctgggttgaggttaaggatgctgctaat |
| | | ctacaatgcacagtacagccaccccacacacacacactacaaagc |
| | | attctcccacccaaattgccaggagtgaggactttgagaaaccct |
| | | gccctagactgttaaattcaaaaggaaataatggtttattgctca |
| | | cagtgtgccaggcactgtgttaaactccttatattcatagttttg |
| | | tttttatcctcacaacaacctgtgaagaaagaactctcatccgtc |
| | | accactttactgttgagggcactaagccttctaaaggttaaatga |
| | | cttgcccagggccgcaactattagatgttgcagtcaggatttaat |
| | | tccaggcactttgtgttcaaagtgtttctcatccactgtgctata |
| | | tgccagtagtgcccaaacctaactttagccagcaattgtctgcat |
| | | ctcttcagtttatgaacatttatttattaggacatgcaggataat |
| | | cataccaacacagtccgtgtatccagaattctaattgatctaggg |
| | | agtgggagagcctgcctctctattttttttaaattggtgtgaaat |
| | | atacataacataaaattggccattttaaccattttaagtatataa |
| | | ttgagtgacattaggtagatttattatattatgtaaatggactta |
| | | acacaatttatttccagaacttttttatcatcccaaacagaaact |
| | | ctgtactcattaaacagtaactccctgtttccccactcccccttg |
| | | ccccagacgctagtaacctccattctacttcccctttctgtgagt |
| | | ctccctgttgtagctacctgatgtaagtgaaatcagaccatgttt |
| | | gatcttttgtgtctggcttattcacttagcatagtattttcaagg |
| | | tttatccatgcgtgcaaattcccttcttttttatgacgaaatact |
| | | atttaattgtgtgtgtacatgtgcacacacaatgaaatactattt |
| | | catcataaaaaggaagtttgtacacatatatacacacacaccaaa |
| | | ttttgtttatccattcatcagttcatgggcatttgcggttttttt |
| | | ccacctttcgactgttgtgactttccgtttatttttaatctgaac |
| | | tttactccatcacttcctccctttccttttttattcgcaccataa |
| | | ttttgaacaagcagactttgtattttcattactctggggattttt |
| | | ttgagggaggcttgctaccctttggggttgtggtaaggttgtgcc |
| | | agtaacagaattcatgcagtaaaaaacattcatgggactttcttt |
| | | tgtgataggtactagggatgcagagatgaataatacaaggtctcg |
| | | accttcaaggagctcagggtttagtagggaaaacagaggtataag |
| | | taagtcattgcaatgccaggtcccagatatgggaggattccagca |
| | | aagagaggataaggccaggcacagccgctcatgcctatagtccca |
| | | acactttgggaagctgagatgggaggatcacttaagcccagcagt |
| | | tcaaggctaccctgggcaacatagtgagtggcaaaaaatacaaaa |
| | | attagccaggcagggtggcacatgcctgtggtcccagttatttgg |
| | | gagtttgaggtgggagaatcacctgagcctgggagattgaggctg |
| | | cagtgagctgtgatcacgccactgcactccagcctgggtgacaca |
| | | gtgagaggtgagagcctgtatctaaaaaaaaaaaaaagagaggat |
| | | cagatttacctggagaggtcagagaaggtttccagagggagtaaa |
| | | acttgagcttcattttgaagaatgagaggggaatacaaggaggga |
| | | aaaataataggaacaagacctgttcctgttacagcaaacctgtaa |
| | | ctcccaccatggaatgggcacgttttcctgtagacatttggagcc |
| | | agtgtgggcttcaagcctagggggctgatatgatcatttgtcaat |
| | | ctgaagatgactcttggaacaacgtagaggatagggtgaggtggg |
| | | taggctggtggctgacagactaggtcaggagagggctgaccaaga |
| | | ggaggaggggtgacgagggctggaactaaggcatatgagctggga |
| | | tggaaagaagctagatagaaaggaaacaaaaaccatcagaactgt |
| | | ggaaccaactccatgggagggataagaagagggcctattctaggg |
| | | tgaaacccaaatttctggcatggtaccattaacccaaacagaaag |
| | | aggagatttccagagaaagaaaagcaaattttgcgggaggtgagt |
| | | gtgagctgtctagagaatatgcaggtagaactacatgttaactgt |
| | | aacatatacttgtctgcacctcaggagatggctgattaagaattc |
| | | aggattcaggccaggcacggtggcacacgcctttaatcccagctg |
| | | cttaagagactgggggggaggattgcttgagcctgggaatccgag |
| | | atcagcctgggcaacatagcaagacactgtctctaaaaaaaaaaa |
| | | aaaattcaagattctggagtcaatattacttaaggtagcaactgt |
| | | ctattctatagaaaatggacaaatatagataaaaagccacttccc |
| | | tttctaaaagtgtccctgcaattcaagtgaatactaggaaggatt |
| | | gtgttcattcttctaaacaagactcacatgtatcttagcacaaaa |
| | | agaggattcttttatgatacaaatgcactgagaatttggtcaggc |
| | | tatcacaatgaactgatagttcagatggatttgagtctttatacc |
| | | actctggaatctggaccaactgggcctcctaaggccattttgcag |
| | | atctgggcttgtttctgaagctacagacaggcctcttccaagcac |
| | | tctaagtgctccacaaataatatttctgtttcccagaacaacccc |
| | | acaaaaaggtactcttactccatttttagatgaggaagtggaggc |
| | | tcatgatgtcaggtaagctttctcagctcccaagtggttaagccc |
| | | tcagtttaatgtcatttgactccagagccctatgttgcaccatgc |
| | | cttgataataggccatatgggtttcatgtatttcagatggggaag |
| | | gttagtgtgaggtgaaagatacacaattaaccttttaaccatgga |
| | | actgaaatatttacagatgaagtgatacaatagctggaattaatt |
| | | ccaaaataattggggggtactggtccatggcctgttaggaactgg |
| | | gccgcagagcaggaggtgagcagcaggctagtgagcattgctacc |
| | | ccctatcatatcagcagtggcatcagatgggtgaggatgtaaatg |
| | | aactaggattggcattgagttgatattgttggatctaggtgatag |
| | | atttatcattataatattctctctttgtgatatttctgatatttt |
| | | ctaaaataaaaagttgtagttatttatttatttagacggagtttc |
| | | actcttttgcccaggttggagtgcagtggtgtgatctcggctcac |
| | | tgtagcctccgcctcccgggttcaagcgattctcctgcctcagcc |
| | | tcccgagtagctgggactacagcctcccgagtagctcaccaccac |
| | | acccagctaacttttgtatttttagtagagaggaggtttcaccat |
| | | gttagataggctggtctcaaactcctgacctcaggtgatccaccc |
| | | acctcggcctcccagagtgctgggattacaggcgtgagccagtgt |
| | | gcccagccaataaaaagtttttgaaaggatttagataaaatagtt |
| | | ggggaaatggcatttttgttcaaagccaattatttatgtttggaa |
| | | tatcttttgtgcttggagttctccattacagagttccccagtgtt |
| | | cctattaataagtaacattgagcagaggaatgcactgtttagatc |
| | | agcagtccccaacctttttggcaccagggaccggcttcatggaag |
| | | acaatttttccacagacctggggtaggaagtgtttggggatgaaa |
| | | ctgttccacctcagatcatcaggcattagtgagattctcataagg |
| | | agcaggcaatctagattcttcacatgcgcagttcacaatagggtt |
| | | ctcactcctgtgagaatctaatgccacccctgatctgacaggagg |
| | | cggagctcgggcggtaatgctcacttgcctgcggctcacctcctg |
| | | ctgtggggcccggttcctaacaggccatggaccagtacccgtctg |
| | | cagcctggggactggggaccctgctttagatgatgtactctggct |
| | | ttgcattctggcattagctaagcaccctcttaaaggaaattgggt |
| | | ctatactctcagtccgtgttctccctaacacctggaaacattgaa |
| | | taccttcaatgctgggaagttaactcccaccacaactagaacagc |
| | | tatgggaaagacaacagttgattttgaagagtgtcaccaatttgc |
| | | acatgattccatccttaaccattcttatcctatcagctctgccaa |
| | | acatggagaatagttggctgcaggacagctatttttcctacttgt |
| | | agatgcaactatttctcacccaccaggatgtaaaaggtccctgta |
| | | ccctaagattggtcctacatacacacccaatgggaaaatgagatg |
| | | aaaaatttaaagcagtaaatatttgaggaagtagatagagtaatt |
| | | tagaaaaagaaaatacacagggccaagcacagtggctcacatctg |
| | | taatcccagcactttgagaggccaaggtgagaggattgcttgagc |
| | | tcaggagtttgaggccagcctaggcaatgtagtgagaccccacct |
| | | ctacaaaaaattaaaaacttagttgggcttggtagtatgtacctg |
| | | tagtttaagaaacttgggaggctgagctgaggcaggaggattgtt |
| | | tgagcccgggtggtcaaggctgcagtgggccatgattgtgccaga |
| | | gtactccagcctgggtgatagagtgagactctgtctcaaacaaaa |
| | | aaaaaaacagagacagaaaaaaagaaagaaaatatatggatgtat |
| | | atcatataaaaatataaataagggaggccaagtgcagtggcatgc |
| | | ctgtaatcccagcactttgggaggctgaagcaggaggatcacttg |
| | | aggccgagaattcgagaccagcctgggcaacgtattgagacctca |
| | | tctctgcaaaaaatcaaaaaatgaggcggaaggatggcttgagcc |
| | | caggagatcaagccttcagtgagctgtgatcgtaccactacactc |
| | | caggctgggtaacagagagagaccctgcctcaagataaataaatt |
| | | catacatacatacatacatacgtacatacatacatacataagaag |
| | | acttgtttctttccatttgcaatgtttcattcaaaggctagaatt |
| | | aaattgccgtaggccatcacaagtttagcttgaatattattattt |
| | | tttcaagatggagtctcactctgtcacccaggctggagtgcagtg |
| | | gtgtgattttggctcactgcaacctctgtctcccgggttcaaccg |
| | | attcttctgcctcagcctcccatgtagctgggattacaggcgccc |
| | | gccaccacacctggctaagttttgtatttttagtagagacagggt |
| | | ttcaccatgttggccagactggtcttgaactcctgacctcaagtg |
| | | atctacccgcctcaccctcccaaagtgctgggattataggcttga |
| | | gccactgcacccagcctagcttgaagaaaatttgataggagtttg |
| | | tttttttctatttataggccaagcaataccacgtataaatattaa |
| | | gaatcatggctgttccttagtgcctagttgtttataaaccatggg |
| | | aaagaatgaaatcattgaccaaatgagacagggtaacagtcttcc |
| | | ctgggagtaaagagactagcagtctctattgacatatattttagg |
| | | cctggcctccaaaataaatttacccaaagaagtgatgtatttgtg |
| | | tccagggctaccgagcctaatctttggctgcctctgtgttattct |
| | | aagttgtaattttccatgtcatctaaatttgtaataattctttaa |
| | | tatgatagtgtttcagtgaacaaacattctgcttgctgcatttct |
| | | tctgagtgagtaattccctgacaactaccagatcttggcagaagc |
| | | aaagttggtcataagttatgctccactctcagtgctggtgaaacg |
| | | tatgatgcgtaacacagtgtttttgaattcagtgctgattttcct |
| | | aaaggacatttggaaggaaaaaagaaaaggaaaggaaatacccaa |
| | | attcaggaatagaacttacatattattataagacttaaaaaatac |
| | | atgaacactaatgatgaactcatttcttcgaagtaaaaggcctta |
| | | tgctattttttcccatttccctatgtggcttgattgtggcgaaag |
| | | tggctgtgtgagtttccattattgaaggagttaaggtctgtggaa |
| | | tcaaagcatgagacaacatgcaaggaccaggttggtttccattta |
| | | acagccaacctaatctaactgaaaggattgagaggtttgtctttt |
| | | ttggaaagtgttaaggttcttccaagtaaccagcaatgtgacttt |
| | | accacactgttcattaacggggctttggatggccaccatgtctgc |
| | | tgctggctgagtccaaaactgcggtcactctctccagcaagctca |
| | | tggagggttacgtcatcttcactcagccagcccaacgctttttcc |
| | | atctgctaaaatgtagaacatggttgttgattctactctttctac |
| | | aaagaaagagagggaacatggatgagttgctgcttttaaagatta |
| | | tatgttaattgctgttttaaaaatctgctcagctaagcacgctta |
| | | gtgtaatcagtcaccatggagttttttaataggacagcttctgct |
| | | cttacagagcagagtttttatgccagtaggttagaagcataacat |
| | | gtttctatattgaagtgattctccaacaaggacttcttttcatca |
| | | ggggtgacctaaaagttattacttgaaatataattacatcatgtt |
| | | cttaactgggtttagtagtaggataattacaaggttgtatccacc |
| | | tcaaataggaagaactgataagttttgcacaattatttaactcct |
| | | ctgttaggccttccttatttcctgttctctttttaaaatttgact |
| | | aagatattgctaatgggcttgggagcctataaaatgatcagaatt |
| | | gttgccttatgttttgcatgtttggggtaacattggagccagatg |
| | | tactcttaaataaaaggtaggcctacaaaaccagtttctcagttg |
| | | cattcaaaatgtgattaaaaaaaaaaaaaggagaatctcccttat |
| | | aggtgaactttttaatttgtgctttattttcccttttgccattca |
| | | tgagatttcttaaataaaatgatatcttttcttttcttcattatt |
| | | attttaaagGTCTCTGGGGAACAAGACTCATGGAGGAAAGCAGCA |
| | | CTAACCGAGAGAAATACCTTAAAAGTGTTTTACGGGAACTGGTCA |
| | | CATACCTCCTTTTTCTCATAGTCTTGTGCATCTgtaagtagaata |
| | | tttccttgcactaatgggaaagttttgaaaagatttgacctatcc |
| | | aaatcataattaaaaggaagtgtgtatgcaccagaggggcaactg |
| | | ggaagttaccttcttacctttgtttttaattctaatatttttatt |
| | | tgggcatttgtttattgactatcttcctatggtagaatgcaagct |
| | | ttataagagaagggacgtgatttgttctctgctgtacccccattt |
| | | cccaaaactgcagatggcaacagaaggctctgaaaaatatataag |
| | | aaagaatttttctaattgtgactaaattgtgaccaaatgctaagt |
| | | gactgtggacttgcgtttaacacaggacgggagaggcaaagagtt |
| | | caattccaatttagaatttggtcaagttctcttctgcactctggt |
| | | aaacattaattaaaaatcagcattatctgaccagccagttcatcg |
| | | tcagtggtggtgattttcactatgagatacgcgtggcaacttgcc |
| | | agacaccaagaaaccaagttagaggatttttgtattagattcctt |
| | | aacaatgaatacagtatcaccattattacagtatcatcattattg |
| | | tcatactattattatatcagttaacataaagtctgcataagaatt |
| | | gtttccagaaaaatgactttccaaatttaactttcaggaaataca |
| | | aataatgctactaatattgcttttattggcgtatacatgtaatat |
| | | ccccttcttttggatttggatatgttgtgtcattgcctcatttta |
| | | attcattatttcttctcaatctttaataattgctggacttttact |
| | | ccacaagaaacttgctataggcccatctctttcgtcttctttcct |
| | | tctttcagttcgtcttcccatcctctggtagggggaggggaggga |
| | | tgcctgagcgagagactagctgtaggaaccatttgtctcaaagtc |
| | | cagaaagccacaggtgatggatttgtcctctgaatcaaagggcgt |
| | | tcgatgatggatttctgtcatgtctcatctaaagtcttcacgaga |
| | | acagatgaggaagcagttttatgaccccagagcctcctaccaaac |
| | | tcctctgagaaaaggtttccttttttttttttttttttaaattag |
| | | agacagagtcttgatctgtttcacaggccggagtgcagtggcaca |
| | | atcatagctcactgcagcctcgagctcctaggcttcagtgatcct |
| | | cccacctcagtcacccacgtagctgggactacagctgcacaccac |
| | | catgcccagctaacttttaaaacatttttgtagaggtggggtctc |
| | | actttgctgcctatactggtctccagctcctagcttcaagtgatc |
| | | ctcctgccttggaagttctgggattataggcatgagccactgcac |
| | | ccagcctggatgtgatatttttatgttttaaattgttagagttta |
| | | gaaacttgagattgagtttgctgcctgcattaaaatgatgcttaa |
| | | acattaaactgcagtggccttaaatattaacaagttgattagaat |
| | | tactaagttcttttcaagctttacatatacagacaaatttcttat |
| | | gcaaaatagaaggtaacccctgtacgtaagtctagaatttcagca |
| | | gtccccaaaactgactgagcattagaatcactctgattttaaaat |
| | | acatatgtggttttccgagatctactaacagagcctccataatgt |
| | | agcctagagaaacgagttttcagatgtagtgataaatttggaagg |
| | | taagtcagagaagtaagctgaagacagagttttaggaaatatgcc |
| | | taaagtcacataatgaattggttttcttgtttatttgagaatatt |
| | | gtcgctttttgttctttttcatgcaaatcacattttatttcttat |
| | | gtgagtagctatatatttaaaaattttgtttttggaatattgtag |
| | | aatctctacttaagaaagtatcttagcagtcatatggtctgacct |
| | | cactgaatgctaaattctctttaaaacatccgtctcagatggtta |
| | | ctcatactgcctctgattgaacaagagtcccaggttaagggactt |
| | | acttctttgaaatcgttcatttcatttttctacagctatgttaga |
| | | aagttcgtccttagcagtgaagccagagtctatctcttataactt |
| | | ctacccagttgcaccctccaagcctacctataccaagtatctttt |
| | | ttccacgttgcttttccaattcagctcttcgcaagtttggagaca |
| | | aatacctaatctcctctaagccttctccaggttaagcctttccag |
| | | ttcatccagctgttgattatgtgattggagacacaagtttgagta |
| | | actcccatgatggaaagtccctctggtgaatgttctgttcatcag |
| | | agtccctaagaaagcacatgagcctcaccatggtgagtggggcca |
| | | tgagattcctaaaccagacactacactgtggcttgtgcaacctac |
| | | catggccagactcatgaggctgtttcatcatataaataattcctt |
| | | agtctcttcaccaaaaactgtgaagcactgtgtcccccagctgta |
| | | tgtgagctagcctgggaccatagtggaggactcctctttcaaccc |
| | | tgttaaatttcatcttgttaggatctgcccatttttccattctgt |
| | | tgaaattatcttggaactggattcattcatctcacatcagctact |
| | | ccccgaagcctcacagtatcagcagagttattatctctatccata |
| | | tccttgtaaattattaaactaaaaaagattggtctaagaacatca |
| | | gtccattaatcaaggctctttggttggagttcacttcattatatt |
| | | atcatccagctcagagtgttcataaacaagatgataggaaagact |
| | | tttccaaatgcctgtctgagtaattcccccattcctgtgatctgt |
| | | gagctgttgaccagattaaaaaggaagtgagaataaccaggcaag |
| | | atttactcctagcaaaaccttactggcttctagtgactgagtcct |
| | | ttccctattgcttaccagctatctttttaattactagttttaaaa |
| | | tcttgccagcaatgtcaatatcaaatgaccaagaatatcaaactc |
| | | attactctgtatgtaaatgagatagtgtacttacccctatggctt |
| | | aagtattaggctgttcttgcattgccctaaatacctgagactggg |
| | | taatttataaaaaaagaggtttggccaggcactgtggatcaggcc |
| | | tgtaatctcagcactttgtcaggctgaagcaggtgtaatggtgag |
| | | ccaagagttcaaacttagcctggacaacaaggtgaaaccccctct |
| | | ctgcaaaaaatacaaaaattatctgggcatggtggcatgcacctg |
| | | tagtcccagccacccaggaggctgaggtgggaaaattgtttgaag |
| | | ctgggagtcagtgattgcagtgagccatgattgcaacactgcact |
| | | ccatccagcctgggcgacagagcaagaccctgtctcaaaaaataa |
| | | ataaatgaataaataaataaaataaataaataaatagaaaagaaa |
| | | aagaaagaaaagagatttatttgcctcatggttctgcaggctgta |
| | | agggaagcatagctccagcatctgcttctggggaggcctcaggaa |
| | | gctgttactcatggcagaaggtgaagcaggagcttgcacatcatg |
| | | tggcaaaagcaggagcaagagagagagaatggggcagggaagaag |
| | | ccccacacttttaaatgaccagatcgcatgagaaatcactcgtta |
| | | cctcaaggacagtaccaagaggatggtactaaattcctgagaaat |
| | | ccacccccatgatctgatcacctcgtaccaggccccgccttcagc |
| | | attggggattatgtttcaacatgagatttggatggggacaacatc |
| | | caaactatatcaccttgcatagtaggtagggttttaaaaagcagt |
| | | ttggcacagtaagaaaagtacagattttttttgcatcagacagac |
| | | ctgagttaaaatcccagcttcactgctaacatgctaggtaaatgt |
| | | gggcaagttaattaacatttctaagcctttgtttcctcactggta |
| | | aaacaagtatttggaaatatcattgtgaagattagaaataataca |
| | | tgaaaagatcctaggatgctgtctgtcatacagtagtagtagtaa |
| | | gaagttattcttgccaaagattgttgagaatggcagaattatctc |
| | | agttctaagagctatagtttctaattatttgagcctagactcaga |
| | | ttcatttggagcagctaactgctcaccaagagcttattttccatc |
| | | ttaccaatgaggttatgtgccctgtgtttttaaaatcagtctact |
| | | taaccaagagaacagaaatgacatgagaattaagtaatctcactt |
| | | tctctgttatttaggatttattcctactcaaaacctgagagttgc |
| | | tatgaattcaccattaaagcacttattaatatacatgggttactg |
| | | ttataaatagcaatagtattgctattgtgtgagttaggtgttgaa |
| | | gttcaagaaaggaataaagaatatttagaagatctttgaaaacag |
| | | tgtctgggtacggtggctcatgcctgtaatctcagcactttggga |
| | | ggccgaggcaggcagatcacttgaggtcacgagttcaagaccagc |
| | | ctgggcaacttggcgagacctcgtctctacaagatatacaaaaat |
| | | tagccgggtatgttggcatgcacctgtaatcccagctacttagga |
| | | ggctgaagcacaagaatcacttgaacctgggaagcagaggttgca |
| | | gtgagccaagattgtaccactgcactccagcctgggcaatagagc |
| | | aacactctgtctcgaaaaaaaaaaaaaaaaaaaaaaaaagaaaga |
| | | aagaaggaaggaaggaaagaaaaaaaaaggaaaaaatgcaaggaa |
| | | ggtatttggtgaatctataataataaaaatgtatttgtcatttcc |
| | | tttttctgtgctctcattctataaaattgagtaaaaaatctatat |
| | | atagtttaaacacattaatagaaatcacaaaagttagctgagtca |
| | | acattgtagaaacataatatttctgtatgtcaaagaaatagacaa |
| | | cattaaaaagcagaaagcaattacaaagaatgattataagaaaca |
| | | tcacaaagggttaatattttaacacatttgaaactcaaaaatcac |
| | | tgagaaaagcagtagacttccataaatattttatagagtagaaaa |
| | | aataggccaagcacagtggctcatgcctgtaattccagcactttg |
| | | ggaggccgaggagggtggatcacgaggtcaggagttcaagaccag |
| | | cccggccaagatggtgaaaccccatctctactaaaaatacaaaaa |
| | | ctagccaggcgtggtggcaggtgcctgtaatcccagctacttggg |
| | | aggctgaggcagggaattgcttaaacccttaaacccgggaggtgg |
| | | aggttgcagtgagccaagttcgcaccactgcattccagcctgggc |
| | | gacagaacgagactctgtctcagaaaaagaaaagaaaagaataga |
| | | aaaagaatccatgggcaggcacagtggctcatgcttataatccca |
| | | gtactctaggaagccaaggtgagaggatcaattgaggccaggagt |
| | | tcaaggccagcctgggcaacatagcaagactttgtctctattaaa |
| | | aattttaaaattagccaggcatggtgacgcacacctgtagtccca |
| | | attacttgggagcctgaggcaggagaactgcttgaggctgcagtg |
| | | agctatgattagaccactgcactccagcctgagctacacagtgag |
| | | accttgtgtcaaaaaagtaaaaaaataaaaattagccaggcatgg |
| | | tggcacatgcctgtagtcccagctactcaggaggctgaggcaaga |
| | | ggatgacttgagtctggaagatggagactgcagtgagctgtggtc |
| | | atgccactgcactccagcctgggtgacagagcaagaccctgtctc |
| | | aaaaaaaaaaaaaagaaaagaaaagaaaaataaataaaatttatt |
| | | caaatacaaaagtgatgtggtttgactctgtgttgccacccagat |
| | | ctcatctccaattgtaatccccgtgtattgacggaggttcctggt |
| | | aggagatgattggatcatggggatggtttcccctctgctgttctc |
| | | atgatagtgagtgagttctcatgaaatctggttgtttggtaggtg |
| | | tctgtcacttaccccttctttttctctctcctgctgccttgtgaa |
| | | gaaggtacttccttctcctttgccttccaccatgattataagttt |
| | | cctgaggccttcccagccatttggaactgtaagtcaattaaacct |
| | | ctttcctttataaattaccgagtctcaggcagttttttatagaag |
| | | tgtgaaaatggtctaatacagagacttggtaccaggagtggggta |
| | | ctgctataaaaaataacctgaagatatggaagcgactctggaact |
| | | gggtaacaggcagcaattggaacagtttggagggctcagaagaag |
| | | acaggaagatgtgggaaagtttggaatttcctagagacttgttga |
| | | atggctttgaccaatacactgatagtgatatggacaatgaagtcc |
| | | aggctgagatggtctcaggtggagatgaggaacttattgggaact |
| | | ggagtaaacgtcactcttacatgttttagcgaagagactggcagc |
| | | atttttcccctgccctagagatctgtggaactttgaacttgagag |
| | | acatgatttagagtatctggcagaagatatttctaagcaccaaag |
| | | cattcgagaggtgacctggcttttcctgaaagcatacagttatat |
| | | gtgctcacaaagagatggtttgaaattggaacttatgtttaaagg |
| | | ggaagcagagtgcaacaaaagtttagggagtttgcagcctgacca |
| | | tgtggtagaaaagaaaaacccattttctggggagaaattcaagct |
| | | ggctggagaaatttgcataagtaacgaggagctgaatgtgagttg |
| | | ccaagacaatggggtaaatgtctccagggcgtttcagaaaatctt |
| | | cagggcagaccctcacaacacaagcctggaggcctagaagggaaa |
| | | aatggtgtgagccaggcccaggcccaggccccagctgttctgtgc |
| | | agccttgggacatggcaccctgtgttccagccactccagctccag |
| | | ctgtggttaaaaggagccaaggtacagctggaccattgcttcaga |
| | | gggtacaaatcccaagcattagcagcttccatgtggtgttgggtc |
| | | tttgggtgcacagaagacaaaagttgagctttggaagccgctgcc |
| | | tagatttcagaggatgtatggaaacacctcgatgtccaggcagaa |
| | | gtctgctgcaggggcagagccttatggagaacctctgctagggca |
| | | atgcaggggggaaatgtggggttggagctcccacacagagtcccc |
| | | actggggcactgcctcatggagctgtgagaaaaggaccaccatcc |
| | | tccagactccagaatggtagatccaccaacagattgcactctgcg |
| | | cttagaaaagctgcaggcactcaatgccagcctgtgaaagcagct |
| | | gcaggggctgtaccctgcagagccacagaggtggagctgtccaag |
| | | gccatgggagcccaccccttgcattagcatggagacaggggatca |
| | | aaggagattttggagatctaagatttaatgaatgccctgtcgagt |
| | | ttcagacttgaatggggcctgtgacccctttgttttggccaattt |
| | | ctcctatttggaatgggaacatatacccaatgcctgtacccccat |
| | | tgtatcttggaagtaactaacttgcttttgattttacagactcag |
| | | gcagaagggacttgccttgtctcagatgagactttggacttgaac |
| | | ttttgagttaatgttggaacgaattaagacattggggttctgttg |
| | | ggaaggcgtatttggttttgaaatgtgagaaggacatgagatttt |
| | | ggaggggccaggggtagaatgatatggtttgactctgtgtctcca |
| | | cccaaatctcatctccaattgtaatccccatgtgtcaagggaggg |
| | | acctgatgggaggtgactgaatcataggggcagtttcccccatgc |
| | | tgtttgcatgatagtgagggagttctcatgagatctggttttttg |
| | | gtaagtgtctgggcttcccccttttccctctctctcctactgcct |
| | | tgtgaagaaggtacttgcttctcctttgccttctgccatgattgt |
| | | aagtttcctgaggtctccccagccattcagaactgtgagtcaatt |
| | | aaacctcttcctgcctattctcaggcagttctttatagcagtatg |
| | | aaaatggactactacagaaagtgtgtaactttaaactcagtagta |
| | | tccaaagaagtaatgaaaatggagaaacgaacaacaaaatcatag |
| | | tacaatatggtgtatgtactaggacaggaagagcccttttaagaa |
| | | gagatctatgtatttccatttgtttatctctgaaagaaagcaact |
| | | ttgccttgtattctgaaaaagaaaggaatattttattttacttgt |
| | | aaaaatcttacaaggatgctagtctaaatatagttttcctaattt |
| | | gccagagaatccatgaagatcgagttgataacaagatcagtgaag |
| | | taaaggtcagtgagttaatctcacagcagctgcaggctaattcca |
| | | tttccagtgaaaaacgtcttgattgctcaccacatatcttttcac |
| | | cacaaacagtttcagtcttaagatcacatgttgcaatccatgaga |
| | | agtaactattaagccttcaactatgactggagggctcctcgccct |
| | | ttctgataaattgactggacaaaaactcaattttaaaatgacaag |
| | | aaatagaagatgtataaatgtactttaaatgtgaccaaaatgggt |
| | | tgtgaaaacacaagacacaatatccaaaaatgctggcaacacagt |
| | | acactgtagagtattggttgtttatttacccttgctattgtgtgg |
| | | ctgagcttactgccactgcccagcattgcaagggcatcaaactgc |
| | | ctatcactagcctaggaaaagatcaaaattcaaaattctaagtac |
| | | agtttctactgaatgcttatcacttttgcaccattttaaagtaaa |
| | | aaaatcagtaagttgaaccatcatatatccaagattgtctgtata |
| | | taaatattatacatctttctctcacttttaaaacaaaataatact |
| | | agccaatactaccattctcaaaagcacttgtgtcaacagccttta |
| | | ccccttaaagattttcctcacaattttaaaattgttacttactat |
| | | tttctttgaaatgttgaccaaacctggattaaaagatttgggggt |
| | | tttagtgactgtatttcacaaactctcttattgattctgcagcct |
| | | cacttctgcctcctaaaaagccctcaccaaggtcacgggggatgg |
| | | ctcttttcagcctcttcctggcatttggtccagtggcatttggca |
| | | ttctaggacttccctctttgtctttgataactccctctcttcctg |
| | | tgttcctccttgctgtgttcacttgcttcgctttcttctttctga |
| | | agcatgtttacacagtgttttctctgattgggcctgtgacgttct |
| | | ttaggtcatcttttccacaaataatgcttcaactagtacttgcgt |
| | | gccagtgactccacgtcccactcatgagctctgaacctagtacca |
| | | gcttctgctggacatctacaatgggatctctcacaggcctctctc |
| | | attggtaacatgccccagcctgaactcatctcccacccatctatc |
| | | cagccatgctctctagttcacctgaacacttgggtgtcatcctag |
| | | atgctttcccttcccagtcttctgtgatcattctgcctcatcaga |
| | | ggctctctaatctgtcttctttcctatatcgctcttgtccctatt |
| | | ttaatcctaatcatctatttcctgacttattcattccttaagttg |
| | | gtcagtaatttaattaaaaacagatttaggccctgaccttaaatg |
| | | tgataagtgatatgaaaggagatgactggggaaaaggatttccct |
| | | caaggaaggcctctgtgaagcctgaagcaagaatgaaaacgagtc |
| | | agacgaagagagaattgtatgaatgaaggctctgaggcaggaaaa |
| | | cactcagatcattccagaatcacttagaagccaagtgaagccagt |
| | | tcctggagagcagatcatcaaatgaagatggaaaggtgaccaggg |
| | | gccagacctgtagttttggtgggccttggtgagggatttacagta |
| | | ggacaccccatggtttaagtatgaaagtgacaagattcctttaag |
| | | ttttaagaggcctcgaaatatgaaccacagattagatggaagcta |
| | | ctctccctgtgtctggactttttagaatttccaagagctgctgtt |
| | | tctggaaccagattaatacaagtcagtcttccatttatttattta |
| | | tgtatttatttgagacagggtctcactctgtcacccaggctgggg |
| | | tgcagtggcatgaacacagctcactgcagcttgggggctcaagag |
| | | atcctcctgcctcagcctcccatgtagttgggaccacaggcacct |
| | | accacccagctaattttatttgttgtagaaatgaggtctcatttt |
| | | gctgcccaggctgttcttgaactcttgggctcaagcgatcctcct |
| | | gcaacatcttcccaaagtgctgggatcactcttccatttaacatg |
| | | ctatctcaacgtcaagataaactttaaaatctttagataataggc |
| | | tggcattttacttaaacgatctttacttcttcagaactgccattc |
| | | cctataaatatctggttcttcaaccacatcaaaccacttgtgatc |
| | | tcaaaaagcctcagcgtacactgtcctttctgtcattctaattcc |
| | | tcctcatccttcaaaatcaactcaaggaccagatccagggagaag |
| | | cttagtggtgcccacccgaaccggccccctccttcgagttgtgct |
| | | gccattcgggcccacctcttcacacagggttgtcagaccagacca |
| | | gctcatgcgttcaccgcccttgcagggatgggatgcagctgtgca |
| | | cctctcagtgccgacacctggagagtctcacgaaatgttgacaac |
| | | atggcctgtttccatttcttgttcactaggacttctcatttacta |
| | | acacacagaatttcctgtagtatgtccacttaatcagttcaagcc |
| | | taataattccttgatttgggtatagtgctttgcatttatatactg |
| | | atggtccccaacttacgatggttcgatttatgatttttcaacttc |
| | | atggtgatgtgaaagtgatacacattctatagaaaccacactttc |
| | | aattttgaattttggtctttttccaggctaccatactctaaagat |
| | | agagccacagatcccagtcagccatgtgattatgagggtaagcga |
| | | ccaatactctacagtgtattgtattgccagatggttttgcccaac |
| | | tagcctaatgtaagtattctaaacatgtttaaggtaggccaggct |
| | | aagctgtgttgctcattcagtaggttaggtatattaaatgcattt |
| | | tcaacttatgatattttcaatttacaatgagtttatcaggatgta |
| | | actctactataagtcaaggatcatcttgtatagcacttttaattt |
| | | atagagtcccttcaaatgtttgtttgtattttatttccactgcat |
| | | ccctgtgaggataccataagttatacagctaacaaaacagttagt |
| | | tttcctgtgcaaagtgatggcttcatcttgtggcagattacctgg |
| | | aatactgtggccaaggcatcttagttctactgtctttatatatct |
| | | agtacagttatatttttatggcagctctgatttcttctttggccc |
| | | aagggttattaagagagggaaaaaatttaatttcttaacagatat |
| | | atatatctatgtcaagtcatatatttaattcaaacccttaatatt |
| | | cctaggtaatttttgtctactttctctgtcaaagattgaaagata |
| | | cagggttttaagtttccaactgtaattgtagttttggtaattgtt |
| | | ttattactaacaggtattgctctgtatattttgatgttctgttat |
| | | tcaatacataagaattcattacagttgaatcatcatgtatagtat |
| | | aatttaccaatgtaaaataacctttttatcaaactgagtattcac |
| | | cttataatgtcctctgtcagaggattataataccacttaaaacct |
| | | tttaaaaaatattttgttcctgataattttagctttgagtggttt |
| | | tcttataacaattctagagatgcgtttttattttttaaccaagat |
| | | ttaaagctttgtgatttagtaagagtctaaaccattcacagttca |
| | | tgcttttttgtcaaatttctattatttagtattttcctctctttt |
| | | atattttcctgttattttccatttctattcttttgttagactgga |
| | | aattttttctttgctttcttttatcctagtgatttgaaatttatg |
| | | taatatatactattctacaataccctttatttgttttcaatattt |
| | | gaacctatatttttcaacattattaagaataaaatagtatttgtt |
| | | gctattttgaaatgatagaccatgtttttaaggcaggttggtggt |
| | | tgttaaggcaccagtatcggccaggcacgatggctcacacctgta |
| | | atctcagcactttcggaggccgaggtgggcagatcgtttgagccc |
| | | agcactttggccgatactgtggtttactgtattttgttctagttt |
| | | attttatggaaaatgggaattcagtggttaagacaaggattaaat |
| | | agcagaagaaagatgtgtacatatgtacagatgtatgtgtccttt |
| | | atatgttttttagtactcttgtctcctttctggtcctcatttaag |
| | | gttatctatttcatgcagtaaatttttcttcacaattcatttcat |
| | | ttagagagtgaatgctaccttccaagtgggctttctccagttttc |
| | | ctttcagggacttaaaggagaagtgatgttaacagttttatattt |
| | | ccattgcattttacagtgtgcagatgtcttcacatatatttcccc |
| | | atttgagctttacaaaagcccttagtattattctcattgtctaga |
| | | ttccaaaatcaggcttagaggagttaaatagttgtccaggatctc |
| | | aagatgcaagacccacaatcatgaacagaggcagatgttcaggat |
| | | ggaggcaagctgaaactcaaaaccaaatcattatgactccaaatt |
| | | caggagtcttttagctgccacctgcatgggctcttggtgtagctg |
| | | accaccagagtttgtagagctgtcattcaggtgtgccatggactt |
| | | tcctgggacctggcacaggagaaggactgagttaatgtttgctga |
| | | ttaaatatctgttacaggctgggcgcggtggctcacgaccgtaat |
| | | cccagcactttgggaggccgagcagggaggatcacttgagctcac |
| | | aagtttgagaccagcctgggcagcatggcgaaaccccgtctctac |
| | | aaaaaatttgaaaattagctggccatggtgatgcatgcctgtagt |
| | | cccaggtactcaggaagctgaggtgggaggatcacatgagcccat |
| | | gagattgaagctgcagtgagctgagatggtgccactgcactccag |
| | | ccttggccatagagccagaccttatctcaaaaaaaaaaaaaaaaa |
| | | gttacaataatcttcccttcaaagctggaaggcattatttacctg |
| | | tctgtccagcagatggtgctacataaccaagggaatctgttgctt |
| | | gcccttggtgaagctattaaagccaatacagatcttgagaatttc |
| | | aaaagcaaaaatcaatactggattatgagtgctctaggaaaataa |
| | | agagataaattttcaatttacatacttatatatagttataccata |
| | | tttgtaaataaaaatatataaataatttatcaaaattccttttta |
| | | acagcaacaaccacagtaaacccacaggttaaaaactccacaaca |
| | | gtctatattaatcagtcaatgcaaagtacattccaattccaagtt |
| | | aactgaaaataatcaacttaatcatttggttggctctgagcagcc |
| | | ttcactgcttgctcttgtgtcatgtttctttctgtcctcgattgg |
| | | ctatagacttacagacggcttttgcagaggacagtgtactcatgt |
| | | ccatcctttgcatcctttgtgggagttggctaggcagcactctcc |
| | | ctggggacactatgaactcctcttcttgagtgcagagatcacgtc |
| | | ttgttcatcttcatgcccaataccttgttccataaatatgaatgg |
| | | attagaattctaaactcttaactctgccccaagacagttctgaga |
| | | ggtagtaagtcatataacacctgaagaggactgttcttgtcctaa |
| | | ttacattaggttataagatgacaggtgagggagccaaaccagggg |
| | | gcctggaaattattcatacatctctagatacagtatacaagttgt |
| | | gtgtattatgtgtatttactctgtaattgattgcttgagatgaac |
| | | ccccaaacacactcgtgtttggatcattattatctacccttctcc |
| | | ttaaataatcttaatttcctatgatgcttgaaagggaaagagggg |
| | | ccaggtgtggtggttcacacctgtaatcccagcactttgggaggc |
| | | tgaggtgggagcatcacctgaggtctggagttcaagaccaacctg |
| | | accaacatggtgaaaccctgtctctactaaaaataaaaaatcagc |
| | | tgggcatggtagcacatgcctgtaatcccagctacttgggaggct |
| | | gaagtgggagaatcgcttgaacctgggagggggaggttgcagtga |
| | | gccgagatcactccattgcactccagactgggcaacaacagtgaa |
| | | actccgtctcaaaaaaaaaaaaaaaaaaaaggcagagtggggaag |
| | | agagctgcatgaaggagagatttactaaatagtacttaatcccaa |
| | | aataatttctataggtttgaatatgatccctgaaatttattatag |
| | | gttcaggtaagtattaatcacgggtattcagaactgtggtttaaa |
| | | aaatgtatagaacatgtttccttccccttgaaactttttatcagc |
| | | taattataggaattatattatacctgcaatcattaaagtccagaa |
| | | tgagacagtacttggtaaagtgctgaaatttataataaatgcatt |
| | | atagcaatccagttaaggaggaagagccaccattattgaacattt |
| | | gttaagtgtcagccattgtactagataaattttagttattatttt |
| | | tatttaggcaccaaaaaatccatgggatagttggttatccccatc |
| | | ttactgaagaggaaactgaagctcagaaagtttaagcaacttgca |
| | | caggtcacatagcaagtaaggagcatggccaggaatcagaccctg |
| | | atctcctttggtctactaagcttgcaaaggatcttcccgcctcct |
| | | tccaagaccattcaatattatcagtaaatgtccatggcaaggatg |
| | | tagttcgagttatagggttccattcaagatatgattggtaggtgg |
| | | gaagcagatatgtctgtgtcaatcagtatcctggaagaaggagat |
| | | gatgaactcaagtggtgattaagggaagtttaatgaagggactat |
| | | ttacagagatgtggtggggttaagagaaccaacaaggggaagtga |
| | | tgcactcaaaaagttactacctccaggctttaggggattggggga |
| | | gggagtggcacagtgtgaacccagtgtggttgtgagaaaagggat |
| | | tccctcagcagccatggccaaggttagagtctccactgccaaact |
| | | gcatccaggtggtgagggaatggagaataggggggggtaacaaac |
| | | tctgacctcggtatccccaaagggcaaaggattccaggtggtaca |
| | | gttcgtaaagattagcctcagggcacagaacagggcagagaagaa |
| | | tggagaattgatctggaggaaacaaacaatggcttgcccatgtta |
| | | ttgcagggagagtaggctggtgtgcacagcaggagggtggggagc |
| | | ccagcatatagctgtgttggggcctgtgcagatcagcctcactgg |
| | | cagggaggatctgagccgagaggtggtggaagatgaaatcgagta |
| | | ggcatgttggtagtcctaaatatcaagtaaacgttcctgatctta |
| | | cattgatactcaatagtaagccaattttgtttcccataagccaat |
| | | attaatattacgtatttcttttataagccagagatatagagagat |
| | | accctagaagaatgataggggaaaggaaggcaagggtgagagaag |
| | | accttgtgtgaatttgtccaaaatgtttatccacaggaacaatcc |
| | | ctttgtgaaggctgctggtatgtgaatgtgtgccggttcccttgg |
| | | ggcgttcatttggatctttctgtgttccagTGACCTACGGCATGA |
| | | TGAGCTCCAATGTGTACTACTACACCCGGATGATGTCACAGCTCT |
| | | TCCTAGACACCCCCGTGTCCAAAACGGAGAAAACTAACTTTAAAA |
| | | CTCTGTCTTCCATGGAAGACTTCTGGAAGgtatttgcaaataact |
| | | ttgaaagtacctctctatcacagaaaattgttcatttggcttcat |
| | | catttcaatgcatgagtatcgacaggacctgctttgcatttaaca |
| | | ctgtgtgagacgtaagttatggtgagttgttagaagttactgttc |
| | | ctactctcaaagggggtaaactaacattgagaactttgcctgtgc |
| | | cttgcactgtgctgagtgtttcatatcttaccttatttaatttct |
| | | atagtctaactctataaggtaagtactaagactatgccctagttt |
| | | gttaatgaggaaaatgagattcaggatgtttaaatgcgtatggtc |
| | | acatggctagggaacaagaaaaattgatttttttctagcctgaca |
| | | gctacttcatcctagtttgtaattcattccatgagtcaagattca |
| | | ataaatatttattgagaatctcctagaatgtaaggccaatgaagg |
| | | gcagtgtggttcttctgtcttgcttcgccttttgtgttttgtctc |
| | | tttgttgatgatggcatgtatccccagctcttagaacagtgcttg |
| | | attcaaagtaagcacattctttcaaaggtctgctgttggtggggc |
| | | ttggtggctcacgcctgtaatcccagcactttgggaggccaaggc |
| | | aggaggattgctttagcccaggattttgaaaccagctgggcacaa |
| | | catagtatgactttgtctctccaaaaaagttaaagaattagcagg |
| | | gtgtggtggtacacacctgcagtcccagctactcaggaggctgag |
| | | gtgggagaatcacttgagcgattgcttgaggtcaaggctgcagtg |
| | | agccatggccatgctactgcattccagctggggcaacagagtgag |
| | | accctttctcaaaaaaaatcccccccaaaaaaaaacccaaaaaca |
| | | aacaaaaaaggtctgctgttgtgaagttcaacccaatccagcccc |
| | | ttcccaagttgtcacaaattccaacgtagttaacagtataccaat |
| | | gagtgataccacaggaaaaatattaaactgatctgagggatatgg |
| | | ggcttggaatctaagaaaattggaagggaaattgaaaaggaaatt |
| | | attatttctccttggggagatagtttctaaaattcttactacacc |
| | | ctggggtcagagctgttgattttaaggatagagacaactgagtca |
| | | caggaaactattcatatataaaagtacctggcatccaaaaccaca |
| | | cttgtataatatgaatctttcaccatctgagtagggcaaatcagt |
| | | ctatctctgttgatcatctgacaaggatagcacactgagaaatag |
| | | atctgtcttccctacaggcatagctagttgtacaaactaacaaga |
| | | gacttttgtatacacattccatgatgataaatgccaatcactaaa |
| | | ggggacgagagggattggagagttcaccatacagcaaaatagtcc |
| | | agacaggtgaaaggtctatcaaatgccaggctggtaatcaaaact |
| | | gtagccttttctctaaacaaagtttagaaccatgattgtgtggga |
| | | cattattttaataagggaaagtgcagttaatcatgaccccacctt |
| | | tagtccaagaacaaaaatcagagctgccacgtattaagtacccac |
| | | tctgtgccaggtgcagtaactatgcaaaagatgggttttccagat |
| | | gcaagaaccttggttcagaggaccctgctcaaggcctcatagcta |
| | | acaaatgatggggcaagatgctatcccaaatctctctgacaacaa |
| | | aactcattcttatcactctactattttcatagagttgccaaatgc |
| | | ttggttatgcaaacgatgcaggcaggggcaagacagcggctgagc |
| | | ttggaactttttcagagatgtttcctttgcttttagTTCACAGAA |
| | | GGCTCCTTATTGGATGGGCTGTACTGGAAGATGCAGCCCAGCAAC |
| | | CAGACTGAAGCTGACAACCGAAGTTTCATCTTCTATGAGAACCTG |
| | | CTGTTAGGGGTTCCACGAATACGGCAACTCCGAGTCAGAAATGGA |
| | | TCCTGCTCTATCCCCCAGGACTTGAGAGATGAAATTAAAGAGTGC |
| | | TATGATGTCTACTCTGTCAGTAGTGAAGATAGGGCTCCCTTTGGG |
| | | CCCCGAAATGGAACCGCgtaagtgtctgtgactcattgccactcg |
| | | gtgatattcattcatttattctctgaactcccaccattcattcat |
| | | tcattccctgacaccttcaccaaggcaaaaataagttcagtgact |
| | | cttcagtgcttatatttaaaccttggccaacttgacctttgactt |
| | | cttaagttttcactacttcttagccttcttttagtttctacatgc |
| | | atatttttcagaagactaaatcgttgaccatataacccctcaaaa |
| | | attaattatctgagcgtttgaaaatttcatttaagatgccctggg |
| | | ccctgtttttacaggtgcagtaacatcatccactaagttatttaa |
| | | cacaagttttctggttcaggaactctttttataggtcttgcaaac |
| | | aggtttttgttcagaatggagttatttaatgtgtaagcttgtgag |
| | | gcaattttttgttaggtttaaagcccattttgttcaaatgtttga |
| | | gattttaggtatatatttgtacacgtgcatatttacaggggcttt |
| | | ttgtacactttggtactcctacttcaaacatcttgtgtattaagg |
| | | gaggtcacttactattttagaagtattgtagttattataaagaaa |
| | | caagaagacctccaaggccgtttcagggtgggcctttgcggttgc |
| | | tgtccctgggtacgtcactggtcggagtcatcttctaagctttgc |
| | | tcagctaattctgtcggttcatctaggttcttttcttggaaactg |
| | | agttgcccagaatccacatttgttactatacaatgggcaatcacc |
| | | ttttcaattagtatattcttcttgtaccttccagtatacactcta |
| | | tttaataccagaacccataagaaacaaatttagtaaaaatccagg |
| | | ttgggcacagtttctcatgcctgtaatcccagcactttggaaagc |
| | | caaggcggacagatcacttgaggtcaggagttcaagaccagccta |
| | | gccaacatggtgaaaccctgactctactaaaaatacaaaaattag |
| | | ctgggtgtggtgacatgtgcctatagtcacagctattcgggaggc |
| | | tgaggcaggagaattgcttgagcccgagtggtgggggttgcagtg |
| | | agctgaggtctcatcattgcactccagcctgggcaacagagcaag |
| | | actccccctcaaaaaaaaaaaaaaacaaatttagtgaaaatccag |
| | | agctttagaacaaaggaactaaatagtctcaaaggacattatcat |
| | | ccaagttatgatagtgatttcgctttctttaaaaaaaaaattatt |
| | | acagatagagtttcttgatgttgcccaggctggcctcaaactcct |
| | | gggctcaagcagtcctccagcctcagcctcccaagtagctgggac |
| | | tatgagaatatgccaccatgcccagctttattttgctttctaatg |
| | | tgcctttttgtagttcctgcaaagcataagcatgccttcatctgt |
| | | ggtaccctttccaatattttatttatctcacatcactaataagat |
| | | aaatttatacagccactgctctgtgccagacattatttaagaagt |
| | | tatttcacgcattatctcatctgccttcacaaaacaactctaaaa |
| | | taggtatcacctccattttatagatgaaaaaactgaggctcactt |
| | | gcccaaagtgtcacagctaacaaattggactgaaccaagatttaa |
| | | gcagcctgactccaaaacccatgtttcgcctactaaacctcttcc |
| | | atattaattcctcctccatattaattgcgtcgttagggtggcttg |
| | | tcgacgctctcagctccccatcagtactcaagcttcctgagggca |
| | | gggattctattttgttaactgctgtattctcaaagccttgaacaa |
| | | tgcctcatatgtaaagatactaata (SEQ ID NO: 231) |
| | premrna | ATGTCTTCTCTCTCTTACAGCTCTTCAAATTCATCAATTTTAACA |
| | ENST0000 | GGACCATGAGCCAGCTCTCGACAACCATGTCTCGATGTGCCAAAG |
| | 0502363.1 | ACCTGTTTGGCTTTGCTATTATGTTCTTCATTATTTTCCTAGCGT |
| | | TGCTCAGTTGGCATACCTTGTCTTTGGCACTCAGGTCGATGACTT |
| | | ACAGTACTTTCCAAGAGTGTATgtaagtatatatgaaattaagaa |
| | | gaaaaatttaatcagagttgtcactgcttctcaagaataaatctt |
| | | catatgaggttgctatatgaccaccaattatttaaaaccagttat |
| | | tttaagtaagaattaattaccttttcccaaaacattgatctaccc |
| | | atgcaaagaagacaatgcatcctgaaatgctgatgcttaagatag |
| | | cagcccaaagtagtaaaatacagttaacagacataggaaaccaac |
| | | actgttctgaagactgagtttttctttgcaccaaatgcagatggt |
| | | agcttctagaaggctgtttgcctatattcttactcctgttgaata |
| | | ttgttgccatatatttagaacttcaagttattttctaaggaaaaa |
| | | aacaagatatttctaatattctaaggtaaactcagaccagtacaa |
| | | gaattttcagtttttttttccaaagatcccaaatgtgaaataaaa |
| | | caacaaaaagcagccagtgtcagatttctatgccatttagaaagg |
| | | agttagtttaaaaaggaatggaagtaatagggttttgtgcataga |
| | | tatctcgaattaatattgctgttgataaaagtgattttgctaaga |
| | | cccagcactgacaacacttggccactttgatcccattttaagtac |
| | | ttgtcagaatattggatctttgaactcaaaccattttgggttttt |
| | | ggggtttttttgttttgtttttttttgttttgttttgtttttgag |
| | | gcacggtcttgctctgttgcccaggctggagtgcagtggtgcaat |
| | | catagctcactgcagccttgaactcctaggctcaagcaatcctgc |
| | | tgcctcagcctgctgagtagctgggactacaagtgtatgccacca |
| | | tgcctggccaatttttaactttttttatgagaagggatctcactg |
| | | tgtagcccagggtggtattgaactccagggcctcacactgtcctc |
| | | tcacctcagcttccaaaagtactgggattacaggcatgagccacc |
| | | acaccaggccctgttgttttttttttaaagaaatttttaacttta |
| | | gaccgagggtgactgttgtcaaggtttagggttaagatgttttac |
| | | ctagattatgtgttgaaatgttatagccaattgctttataagtta |
| | | ttgaataataattgtattttctttttttttttttttttgagatgg |
| | | agtctcgttccatcgcccaagctagagtgcagcggtgtaatctca |
| | | gctcactgcaacctctgcctcccgggttcaagcgattctcctgcc |
| | | tcagcctcccgaatagctgagattatgggcgcacgccaccaagcc |
| | | cagctaatttttgtatttttagtagagacggggtttcactatatt |
| | | ggccaggctgttctcgaactcctgacctcgtgatccgcccgcctc |
| | | ggcctcccaaagtgctgggattacagacgtgatccaccgtgccca |
| | | gcttgtgttttctttttaaccaaatggaaataacctctgtagcat |
| | | gaaagcattttattattattgcagaaggctttaattgctgataca |
| | | agtagcaagactttgtaaatgggattgacaattttctgttattcg |
| | | gcagctacctatactgctaaaaggtccaaaaataatgaaatcatc |
| | | tttaagaaatgttgcatcaactagtggacattctttgtttttgta |
| | | ttgtggtgttttgttttatttttatagCTTCACTCAATTCCGTAT |
| | | CATTTTGGGCGATATCAACTTTGCAGAGATTGAGGAAGCTAATCG |
| | | AGTTTTGGGACCAATTTATTTCACTACATTTGTGTTCTTTATGTT |
| | | CTTCATTCTTTTGgtatgtacatttttatttatagtgaggttcaa |
| | | tttaaacttcgtaaatccttgtcttctcttttctctcacacttta |
| | | tgtcctatcaattttaaataaagacccaggaagtagaaaaaagtg |
| | | tggatgttggaaaacttattttccttttattaattcacagttttg |
| | | agactcatatcaaatgtcttttctgtggtctttcattgatccatg |
| | | tatatgtgtctattcaatgcaaaaaaaattagatctcttccatgg |
| | | tctttcatttctctctctatatatgtatctattccatgcaaaaaa |
| | | gaaattagatcaagtacaaatttataaagatacctaaaatagtgc |
| | | tttgcctaaaaagtagaatatgcttacatgctttttaaactcata |
| | | tgtcagcactttcgtagtcacttgctagcatgacttttctctctt |
| | | tcttcttttctttttaaaaaataagaacggaaaagcaagctagat |
| | | ctaagatgtcgagtaatagttgagtgaatcattgcatgtcaaaat |
| | | taggatattctgttttaaattatttatatcccattcatctagaga |
| | | ctgcctacagagaatattcaaataattaagtttaaaactaaatgt |
| | | aacaatgaatggaaattgcattaaaattattttcaaaaataattt |
| | | ttttattctcttgatttggtacaaatgaacatttttaatgttttt |
| | | gccctaagtcaattaagtttttttaaggtgttttgttctttttct |
| | | taacatttatatattcaattgtctactgagaaggtgttaagccag |
| | | cttaatttaggcaatatttttcatctaaacactaacagtcatctt |
| | | aagaacaattttcttaagaaaataacattttttccatttcagtaa |
| | | attgtgtaaagatcccttgaggaaggttaagtgatcacattttca |
| | | gtaattcagtgtaataactctaaagtcagtccaggtattactggt |
| | | taagtatatggtatttattgattgggtattagatgtactgtatta |
| | | atttcctgtttaaaaaaaattttttttccggggagacacagcctc |
| | | tggtgtaaaacaaaggtgtgttccctagctgtactttaacaggac |
| | | tgaaaaggtcaggaatatcattcaagttcatatgtatcttgctgt |
| | | atgcatggtttatggctcatttttaaacttacacctcttaagctt |
| | | cttcttcctatcatatattaaaacaatggagagaagaataagcct |
| | | ctgttactctaccattgatagtacttcggattctagagtacctga |
| | | atctctactaagaaggcaaaaaccaggaattgagagtcctgcacc |
| | | tgacccttcagttgatctcaggccacctagttttctccgtttatc |
| | | aatctgccaaacaaggatggatagagtcgtggcaactggaaaggc |
| | | tcaaatgtggaattgtttgaatgtggtcctttagtaggcagccat |
| | | cttaccagatctagagtattcagtcatcttaccagatcagtcacc |
| | | agaacatgaaaagaagctcttagtttctatctttatactaaaatt |
| | | gtttttttgtacgactgcacaaaaaagaattgctctccttgcacc |
| | | tcccagagatataggtggatagatacatacatacgtacatacata |
| | | catacatacatacatacatacatacatagacacatacatagatag |
| | | aagtctactttcaatacaaacctgtcttttaaggaaatgacaagc |
| | | tgagcatagggttggccacctttctgagccgattgcctggtatta |
| | | gtttattgcccctgtttagcaagaaggcacagtgttaagaagtgg |
| | | ctcagctgaaccaggataaccccactcttcccccacatcaacagg |
| | | aaagacatcctggtgcagatgtccatctgataattcagggaacct |
| | | cgggagacaggatggagaggagggtgagctagcttcctcttccca |
| | | caccttcaagagcctttctcaagcactttctattttttgaaatct |
| | | ctttagaggtcccagactttgatctgtttcaattaaggtattggc |
| | | aggcattagttaacagccacttggaagcaaaaatagaacattaga |
| | | tccctgagttggaagagagaaggtagaaggtgttacttggactgc |
| | | aattatctgcacttggaattgagcatttagtcaaaaacttatatg |
| | | tattctatattctattctcatttctgctacagaattgtaaacaat |
| | | attcttccttaatacagaaattcatagcccactaaaataagagcg |
| | | ttctcatttgttcatttctcaatcatttaataagtatttactaag |
| | | ccactatatccatatatatatatatatcatatatactgtataata |
| | | cacactgtagtgttttttgtggattgtgtactatgaggtagtatg |
| | | ttagatactgccagtactggggtaaggaaaacagcctgattaggc |
| | | ccttacgaagattcctcagacttgtggggaaaacagacattatca |
| | | aatagaaatacttgcaaaccacagttatgtgttaaaaaggaaaaa |
| | | caaagtaaaaaaaaagttggtgggggggaacctgatctcctggat |
| | | acagtgcttcgagaaagtttgttgttggaaatgcaaaccattact |
| | | actgtggaagggaaaggtcagaaaaatgaactcaccattactgaa |
| | | tagtaatagtagctatcaattaggtggcacttacctgcatcagga |
| | | cctgtcctgagcactttacatagattgtctcactaaccagcccaa |
| | | caaatatgtaagggagatactattatttttcccattttattaatg |
| | | taaaacaattaaataattctttaaaattagacttagaaaagtgga |
| | | gcaacaatcttagcagtgctaggactgaaatccaagtttgcttga |
| | | ctccaaagtctatctctcttccagaaacttttttttactatctgc |
| | | ctagtaggcctgctgtattcctatttgcaacagccttttaaactc |
| | | tttaaaaatgtgtcctgtaaatttcatatatgattatacaaaaaa |
| | | acttggaataagcatacaattctacttatctgtgttaactgttga |
| | | aatttgaagagctttttggaattctatacccttcagtagtgtatg |
| | | taaaagtttctaaatatagagaacatagataagcaaaaataatat |
| | | taaataaaataatcgcaccattagtaggtaaatatactaatattt |
| | | tgttgtattttattcttgtatgttttcacaaagtatatcataaaa |
| | | tttttcctgtggcatgacttaacggagaaaataatcttcccaaaa |
| | | catgtggcagcaaaactgttaatttattacatcaggctgggcaca |
| | | gtggctcacgcttgcaatcccagcactttgggaagccgaggcggg |
| | | cagatcacttgaggccaggagttcgagaccagcctggccaacgtg |
| | | gtgaaacactgtctctactaaaaatacaacagttagccaggtgta |
| | | gtggcacatgcctgtaatcccagctactcaggaggctgagactca |
| | | agaattgcttgaacccaggaggcagaggttgcagtaagctgaggt |
| | | cgcgcccctgcactccagcctgggcaacacagtgagactctgtct |
| | | caaaaaaaaaaatttttttttaaataaataaataataaatttatg |
| | | tcttcataaagcactcagattaggaaaaaaaggataaacaaaaag |
| | | gcatgtgtcatttttttgattgataattccaaattatgtttcttc |
| | | ctttaatttttgccctcctttcatttacaaacagAATATGTTTTT |
| | | GGCTATCATCAATGATACTTACTCTGAAGTGAAATCTGACTTGGC |
| | | ACAGCAGAAAGCTGAAATGGAACTCTCAGATCTTATCAGAAAGgt |
| | | aggaaaaaccttaattctcagaattcttctgtttctgacataaaa |
| | | tgagcattgtttcacccagattttcaaatcaacattgatccattg |
| | | aaattgtttgaaataaagaatacattgctatatttcaggaataat |
| | | ttaaatgttccctatcttggagtcttgatggatatactgctatct |
| | | tgaattttaattctgggaatccttttatgccctggaattaaattc |
| | | tcaacaatcttttgacactttaagagctgagctgaaggttcatca |
| | | ccttcattattttgacatctcctgtagctggctctcacttcagga |
| | | tcctgagttgagaataaactagaagggaagattatataaagggat |
| | | ttccacctcttctgtctcaattaccattttaaaaaaataaaaagt |
| | | tttagaggaaaacacttagtagttcaccctttacccttgaccttc |
| | | cacggcagttttaaaataagcaaaggaaaagattcatgaattcag |
| | | gccatagcctggggcctgagaacttttacttatgcaccttctcag |
| | | gaagggtttcattgttaaatagaagggcaggacaggaaagttggg |
| | | cctctttgttcttctcaatgtaacttctttatttggtttaaagta |
| | | taaaatgtatacaacaacaaataaccacatttaaaatacacagtt |
| | | tgtttcccaacatcattttgctaagtcatagtggctccttaactg |
| | | taatttttttttttattagtccaagccttaggattatgttatctg |
| | | tgatatatgttataatagaaaacttaagcctcttaaaacaaagtc |
| | | cttgggatgggacctaagattcacattatcttgattccgcataac |
| | | agttgcttacattttagcaaatctccagtgtgtatgcaagcactc |
| | | ctcacttggcacaattctgatacacacaaactttggttaccccag |
| | | ttttgttatgtaacaccaccttcaacaacacagttcaaatttcag |
| | | ttatcatagtatattaactctgagtaacagcacaaagtacaaact |
| | | ccactgctagctcttcagtgtatagatcagttacctgagtaacag |
| | | atgtgcaggctgagcaggctcactggtcagtcatgacactatttt |
| | | cagtctgttactaattggtcactgagcatctgctatccaattcac |
| | | aaacaaagaaagcatgtagtgttgcttccttgtgttccagtaata |
| | | agcccatgtgacattttacaaaaatggataattgaaaaagagaat |
| | | gggtgcagtggctcacgcatgttgggaggccaaggcggatggatc |
| | | acctgaggtcaggagttcgaaaccagcctggccaacatggtgaaa |
| | | ccccatctctactaaaaatacaaaattagctgagtgtggcgacag |
| | | atgcctgtaatcccagctactggggaggctgaggcaggagaatcg |
| | | cttgcacctaggaggcggaggttgcagtgagccgagatcgtgcca |
| | | ttgcactccagcctgggtaacaagagcgaaactccgtctcaaaaa |
| | | aaaaaaagaaaagaaaagaaaagaaaaagagaatgggctagcaaa |
| | | gaaatgaaaaatggtaacactggaagtgaaaatcaaaacagagta |
| | | atggatttatagaagaaatagctgagtgaagaagaaataggagtg |
| | | ttgacactgcgatcattcaagagatccagatatggagccagaaga |
| | | acttagggcaggtctatcaacttaaatgaggaaaatagctgtgat |
| | | aaaacagatgaagatgtcttgaggaaatgatgcctgcaaaaaact |
| | | tcacattaagggaagtcttattagagatatttcacaataatgaaa |
| | | gtacaaaagaaaaaatgttggggctggacatggtggcttactcct |
| | | gtaatcccagcactttgagaggccaaggtgggtggatcacttgag |
| | | gccaggaagtcgagacaagcctgatcaacatgatgaaaccccgtc |
| | | tctattaaaaatacaaaaattagccagacatgatggtgcacacct |
| | | gtaattccagctactcaagtggctgaggcacgagaattgcttgaa |
| | | ccagggaggcggaggttgcagtaagctgagattgcaccactgcaa |
| | | tccagccaggtgacagggtaagactgtgtctcaaaaataaataaa |
| | | agaaaaatatgttggaagctcatccacatttaagaaggaatatga |
| | | caattcactaatgcatagaaaagaagttcactccacattgtaaag |
| | | tgtacagtgtaatattatacaatgaaaacaaggcaagtgctgttt |
| | | aaactactctggatacattttttacaaagaaataaaacactttag |
| | | tttttaatgtttctaatgttttacattttagtgtattaaatcaat |
| | | attagttttcttcttttttaagctccctatacatttataactgac |
| | | actaagggagtgtttaatgttttgattaaaagttgtaaagatcac |
| | | agaacaattgtaattcttcccactgattattcagatcattttgca |
| | | caatttcagcttgcatggtcacttacagtgccgcactatgtgcaa |
| | | agcaaggtcaggtctaaagttcgctaatgaaaaatcctcggccag |
| | | gggcagtggctcacccctgtaatcctagcactttggaaggcgagg |
| | | caggcagatcgcttgagctcaggagttcaacaccagcctgggcaa |
| | | catggtgagaccctgtctctacaaaaaaaaaaaaatagcaaggcg |
| | | tggtgactcacacctgtagtcccagctacttgttgggggctgagt |
| | | tgggaaaatcacttgagctcaggaggtcgaggctgcagtgagcca |
| | | gaatcacgccactgcccttctgcctgggtgacagagtaagatcct |
| | | gtctcaaaaaaagggaaaatcctcatctacatttcactgggtttt |
| | | ttgtttgtttgtttgtttgtttatacacacttaaggaaattactg |
| | | tctagaagatagataatataaaaaataaaaatgcaattcatgatt |
| | | cgggtttcttggtattcctaagaactgttgcacagtactttatgc |
| | | tctgaggcagacagctatagcatatatagtaatttttgtttctat |
| | | cacataaacttgaatacacatatgagtaaaagacctttagttctt |
| | | catgacttactgaaagaccctgactttttccatgtaactgttcca |
| | | caagtgttttatggaaaactggatacattaattcttcattcatcc |
| | | agcacgtacttgttgaatggccaatgtacaccaggtttgtagtag |
| | | ttactactgtgaatggaaagtaaaacagatgcaaaaggagaatac |
| | | actaaaccaagtcttttattttttctctctctgatagGGCTACCA |
| | | TAAAGCTTTGGTCAAACTAAAACTGAAAAAAAATACCGTGGATGA |
| | | CATTTCAGAGAGTCTGCGGCAAGGAGGAGGCAAGTTAAACTTTGA |
| | | CGAACTTCGACAAGATCTCAAAGGgtgagaatcatgcttcctgag |
| | | gttctgaaaaattcctgcttctaaagataaattcctggtgataag |
| | | agtatttctagcccaagggctcatacagatacttttttttttttt |
| | | ttccagaggcaggtatctttctggaacatgttataagaggaaaac |
| | | ttgcccccatttggtgatttctcctttcctcctgcattttgatgt |
| | | ctctgtgttgagggtgaactgggtacaaggaatgatttttatctg |
| | | tatcctctctctaatttcagGAAGGGCCATACTGATGCAGAGATT |
| | | GAGGCAATATTCACAAAGTACGACCAAGATGGAGACCAAGAACTG |
| | | ACCGAACATGAACATCAGCAGATGAGAGACGACTTGGAGAAAGAG |
| | | AGGgtgggtctggtttaggagaaccggatttgatttggtacctac |
| | | aacaccacagatgtatcaaacactatagaagtagtgggttattga |
| | | gtctcttgcccattccccaccacactctctctctctctcagtcgg |
| | | tttatgtgttagtaccctgtttattccagaaagaatatataacac |
| | | aattatgtataaaaatgggtggttagcatgatataaaaacgtcaa |
| | | aatgaaaagcaagcaaaacaaaagtaaaaataatggattattaat |
| | | gaagcttaaaaatgcattcataaaaacacatatgcttattaagat |
| | | tgggctacaaattgggccctaagcttgctggtaatcagcttgaaa |
| | | agagaagcctgattagctgcagagtccacaatgtccgtgagagtg |
| | | aagaaaacaaaaaatgacttaccaagagatgtgaaattattctgg |
| | | ttagttagtggctatttaaattgttaacttttttttctttttttt |
| | | ttttttttgagatggagtcttgctctgcctcccaggctggagtgc |
| | | agtggcacaatcgcgactcactgcaacctccacctcccgggttca |
| | | agcgattctcttgcctcagcctcccaagtagctaggactacaggc |
| | | acatgccatcatgcccggctaatttttgtatttgtagtagatatg |
| | | gggtttcaccatgttggtctcaaactcctgactgcaagcaatctg |
| | | cccaccttggcctcccaaagtggtgggattacaggcagtagccac |
| | | cgtgcctttcctaaattattaacatttataataaaattaacagcc |
| | | gccttccatttgaatactttttacaaaatagttaaaaataaacat |
| | | aagtgggcttttatagtcagaaaaaaaaattcaaagctttaccat |
| | | taactttcaaaaataaatggttagacagcaacaacaaaaatctgt |
| | | ggtaactgaggtacagagaacacagatgaatgttattacaaaagc |
| | | cactttcctatgagaagtctaggacagtggtttctaaatgccact |
| | | ccacagacagtgctagtaggtgacagacttctccagtcacagtga |
| | | aatttaagcataaagaaaatgaggaaaatttttacaaggctctat |
| | | ttagacaaagttcttattctgacattacatctttcctactttgga |
| | | gctgttgaatgtattatcttttatgaaaagaaggcgatccaggtt |
| | | gagcatccctaacccaaatatgtgagtctgaaatgctccaaaacc |
| | | taaaacttcttgagcacaaacatgatagtcaaaggtcatgcttaa |
| | | aggaaatgctgtcattggagcagtttggattttgggttttcagat |
| | | tagggatgctgaaccagtaagtataatgcaaacattccaaaatat |
| | | ttttgaaaatcccaaatccaaaacacttctgatcccaagtatttc |
| | | aaataagggatactcaacctgtaatatatttcttcatttctttat |
| | | ttattttattattattttaagatggctcatggcccactgcagcct |
| | | caaactcctaggctcaagtgatcttccgacctcaacctcccaggt |
| | | agctcaggtagctgggactgcaggcatgcatcaccatgcctggct |
| | | aattttttaaaaaattttttgtggaggcagagtctcaccttgctg |
| | | cccaggccagtctcaaactcctggcttcaagcagtactcctgcct |
| | | cagcctcccaaagtattaggattacaggtgtgaccactatgcctg |
| | | gcccatatttcttcatttagttttttctttgcctgctgtgttttt |
| | | aatgttctttcttgttcaaacaaaaagttggctattccttgctgt |
| | | tagttaaatttgccaatctatgaaactgaaaaatgcaggagtccc |
| | | agcctggtgttaaatacaaagaaatcccaggtaaatggcatgcac |
| | | ccagttcctgcttgcccaagtccttggtgaggcttctgtggggtc |
| | | tcagtgttctgctcctcactcagtgaccccttgttcttcagGAGG |
| | | ACCTGGATTTGGATCACAGTTCTTTACCACGTCCCATGAGCAGCC |
| | | GAAGTTTCCCTCGAAGCCTGGATGACTCTGAGGAGGATGACGATG |
| | | AAGATAGCGGACATAGCTCCAGAAGGAGGGGAAGCATTTCTAGTG |
| | | GCGTTTCTTACGAAGAGTTTCAAGTgtaagtataaaggaattggc |
| | | agaatttgcgttgacaagagtccacatgagaccaggcagttccct |
| | | catctctctgaattcactcctttccattactaatcatccagcttt |
| | | taaaaataacttatactggccagacgcagtggctcatgcctgtaa |
| | | tcccaccactttgggaggccaaagcaggcaggtcatgaggtcaga |
| | | agttcgagacgagcctggccaacatagtgaaaccccatctctatt |
| | | aaaaatacaaaaaattagctgggcatggtggtgggcacctgtaat |
| | | cctagctacttgggaggctgaggcaagagaattgcttgaacccgg |
| | | gaggcggaggttacagagagccgagatggctccactgcacaccag |
| | | cctgggcgacagtgcaagactctgtctcaaaaaaaaaaaaacttg |
| | | tcaattggtgttttgtttcttacataatatgtttactataaaaat |
| | | tagcaaataagagcaaaagaaaacattaacatttcacatatttct |
| | | accaatgaaaaatgtttattaatatatcagtgtttgtgtctctat |
| | | ttgcatgtgtttatcaatgtttctatatatttttatggcctaaca |
| | | tatggttcgtcctgaagaatgttccttgtgcatttgagaagaatg |
| | | aatattctgctgttcaagtgttctgtagatgtttgttaggtctag |
| | | tttgtttacagttttattcaggtctcccatttcctggttgatctt |
| | | agatgtgcctagatgtggtattcacgtttgaaagtggggtattaa |
| | | agtctccaagtattattagttggagttcatcccttcaattctgta |
| | | aggttttgctttgtgtattttggaactctgttgttgggtgcatac |
| | | atatttataactagtatattttcctaatatattgaccctattttc |
| | | tctctcaacttaatgaggctaaagaaaaaaaagaattgaccctgt |
| | | tttcattacaagatgttatccactttatctctagtaaaattcttt |
| | | gttttaagtattttttgtttgatattactgtaaccactccagctt |
| | | tcttttggttgctgtttgcatgataaatctttttccatcctttta |
| | | ctttaaacttatttatatctttcagtctgaagtatgtctctcctg |
| | | tagacagcatataattggatcttatctttttatccagtttgacaa |
| | | tttctgtttttgattagattgcttaatccattcatttaatgttat |
| | | cattgatgtagttgatttctgtctgctattttattttttgttttc |
| | | tagcttacttttttttgttcctctttcactgctttcttgtacatt |
| | | aagtgaatattttcaagtataacatttaaatttttttaatgattt |
| | | ttcattttttttagtcaggagttgctctaagacttagtttataca |
| | | attaagttatgaaaaattacttcagatatatattaactgaatcca |
| | | gtgagatatagaaatcatttctatttagcttttttcctcttccct |
| | | ctttttgtgctatatattcatctatctatatgtatatatagtcat |
| | | ctacatatgttgcaaatcacattgttagaacaatgttacattttt |
| | | ataacacactgtgtaatatatagtatataattttatatctcttaa |
| | | tgaagctgagagaagaggaagtatatatttataaatgtgtatatt |
| | | aacctacttttttaccatttctaattctcttcttttgctcctctg |
| | | gattcaagttatcatctgtcgtcattctctttctccgatacagct |
| | | ttgctactgcctacctcctattattgtcaaatatattacatttct |
| | | attatagaccttcagatccaattatgtacatatttttacacaact |
| | | gctttttaaatcagttaaggaacaaaaggagaaatgtacatatat |
| | | actatattttatacctacacagttatctttaccagtgttctttgc |
| | | cttttcatgtggattctgattactatctggagtcacttgctttca |
| | | gcataaagaatttcctttagtattttttgtaaagcaggtttgcta |
| | | gcaatgaattctttcattttttgtttatctgagaatgtttttctt |
| | | tctccttcatttcctctggcttgtattgtttctgatgagaagaca |
| | | ggtgctaattttactgtggtccccttgtacatgatgactcaattt |
| | | tctctcaccactttcaagatttttttgcttttgtctttcattatt |
| | | tttactgtgatatgtctgggtataaatctctgagttcatcctact |
| | | tagaaagtgttttttctgcttctttcactttctcttctcctttgg |
| | | gacccgcattatgcatatgcttaggggtatcacatatttctctta |
| | | ggctctgttcgtcgtcattttttttccctctctgttcctcagagt |
| | | gcatagtctgtattgatgtatcttcaagttcactgacttgttctt |
| | | ctgtcagcttgcttaaatctctgttgagctcctctagttatttat |
| | | ttatttatttatttattttatatatatatatatatatatatatat |
| | | agagagagagagagagagagagagagaaagagagagagagagaga |
| | | cagggagacagggtctcactccatcacccaggctggagtacagtg |
| | | gtgtaatcatggctctctacagcctgaacacctgggctcaaatga |
| | | tcctcctgcctcaacctcccaagtagctaggactatgggcacatg |
| | | ctgccatgcctggctaatttttaaaaaaaaatttgtagagatggc |
| | | atcttgttatgttgcccaggttggtctcaaactcccggcctcaag |
| | | tgatccttccgcctcggcctcccaaagtgctggggtcacaggtat |
| | | gagccaccgcacttagcctgaattttttatttattatacttttca |
| | | actccagaatttctatttggttctttttagcaatctctgtctctt |
| | | tattctgtatttgatgatatcctgtatttgatgagacaatgtcat |
| | | cataacttccttttttttttaagagatagggtctctctctgtcac |
| | | ccaggctagagtgcagtggcatgatcctagctcactgcagcctcg |
| | | aactcctgaactcaagcaatcctcccacctcagcctcctaagtac |
| | | ctgacactacaggcatgagccactgtacccagctaatttttattt |
| | | tttgtagagatggggtctaagttgcccaggctggtctcaaactcc |
| | | tgggctcaagtgatcctccctgctcaggctcccaaagtgctggga |
| | | ttacaggcatgaaccactggacccagcctcctttatttctttaac |
| | | catggtggggttttttattatttgtttgtttgttttaacctgttt |
| | | gaatatatttataataggtaccttcaggtctttgtctgctaagtc |
| | | tgacatctgggccctctcaaagacagtttcagttgtctttttttt |
| | | tttcttcttgtgtatgggaacattttcctgtttttttgttttgtt |
| | | ttttgttgttttgttttgttgttgttgttgttgttaaagattgga |
| | | catgttagataatatattgtaggaactctggctagtgattccctt |
| | | tcctccccaggacttgtttttatttctctttgcttgtttactttt |
| | | tcatacagtctgtttccccacagtgtctgcctctgactttattcc |
| | | ttagagggcgcagctgtggccatgtacgtagtcactctgggaaga |
| | | cagtggttttagcagcgtgctcattaactttctctgacctctttg |
| | | ttatacctcctgcctctgtggatattagacccagttattacattt |
| | | cattgtttactgattggtctattgttttccagaatgccttgggat |
| | | gtaatttgctccacagtctgatccacttaaattcaggcctctttg |
| | | cagggctagttttagaggccagtcttttttttttttttttttttt |
| | | ttttgagacagagtctcactctgttgcccaggccggaatgtagtg |
| | | gtgcagtctcagctcactgcagcctcaatctctcaggctcaagca |
| | | atcttcccacctcagcttctcaagtagctaggactacaggtgcac |
| | | gccaccatgcctggttaatttttgcattttttatagagatggggt |
| | | tttaccatgttgccccggctggtctcaaactcctgagttcaagca |
| | | atccacccacctcagcctcacaaaaggccgggattacagatatgc |
| | | accaccacgcctggcccctgaggccagtctttaactgtcttctta |
| | | gctgtctctttccctggttctctctggtgaactagctggtaattt |
| | | gtttatctcataaggctaccagattccttgtaaatgcttatcccc |
| | | acaatctccattgttttcaagagcatcgttagtctttaatttcct |
| | | cacgcttcattccaaataaagtccattcacttgagaagagttcta |
| | | tattcctatgacctgtgtctccccatgagcaaaactgctactgct |
| | | ttacagagccagggacagtggcccacctctctgtggcatcctgct |
| | | ttatgaacaagtcactgggctcagatggcagtctctgattttctc |
| | | accttgcttcttctggcatggaaactccaccctacaagtgggaac |
| | | tgagtggaagaagagagccccattccccttagccactcttaacag |
| | | gattagaacctctgcaacatgcatttaagaatgggaacaggctgg |
| | | gcacagtggctcacgcctgtaatcctagcactttgggaggtcaag |
| | | gcaggaggattgcttgagccaggagttcaggaccagcctgtgcaa |
| | | catggtgagaccctcatctctacaagaaatagaaaaattaactgg |
| | | tgggttgtgtatacctgtaatcccagctactcgggagcctgaagt |
| | | gggaggattgcttgaacctggaggcagaggttgcagtgagtcaag |
| | | attacaccattgcacgccagcctgggcaacagagtgagactctgt |
| | | ctcaaaaaaaaaaaaaaaaaaagagtaggaacattgaggctgggc |
| | | atggtggctcatgcctgtaatcctagcactatgggaggccaaggc |
| | | aggagtatcacttgacgctaggagttcaagaccaacctgggtaac |
| | | atagcgagactttgtctctattaaaaaaaaaaaaaaaaaaggtgt |
| | | aaaaaacttttgtaacaagagtgggaaagccgggcacagtggctc |
| | | acacctgtaatcccagcactttggaaggccaaggcaggcaggcgg |
| | | atcacctgaggtcaggagtttgagaccagcctggccaacgtggtg |
| | | aaaccccatctctactaaaaaatagaaaaattatctgggcatggt |
| | | ggtgcacacctgtagtcccagctactcgggaggctgaggcaggag |
| | | aatcacttgaacctacgaggcagaagttgcagtaagccaagatca |
| | | cgccactgcactccagcctgggcgacagagcaagactctgtctca |
| | | aaaaaaaaaaaagagtggaaatgttaggatgagaaatgctggcag |
| | | cctgcccctccctgggagatactgtagccctagactggaagttgg |
| | | gggaggagggagccctgtgctcttagctgcacccatgtgaagttg |
| | | tgcttctatcacatgagctggggacaggagagaaggctcagatta |
| | | tggcttcagtgccacagaatctcttcatactaaaatttagtagat |
| | | tttcttgaataaatgctttttcatttgctgtacacccttaaaagt |
| | | ttctagaaatttttaatatttgagttttaaaaaataattttcaac |
| | | agttacagttatttcactaaagagagagtctacagaaccctcttg |
| | | ccaccattgcagaggttgtctttggcttacgagtttttaaagtat |
| | | ttgtatacattttttaagttcaaaataatagaattgtaagtgaac |
| | | atgctgttttcatactgtttttcaagctttatttaatatattgta |
| | | aatctaattctattttattaaatagtctgccacagtataatgtct |
| | | gatgtctccttagaattttattgtatggatgaacaatgattattt |
| | | aatttcctaccaattgttgggtgttttttgtttgtttgtttgttt |
| | | ttgagactgggtctcactctgtcacccaggctggagtgcaggagt |
| | | gcggtggaatgatcacggctcactgcagcctcaacatcccaaggc |
| | | tcaggtgatccttccacctcagcctcgcaagtagctgggagtaca |
| | | ggcacatgccaccatgcccacctattttttagagatgaagttttg |
| | | ccatcctgcccaggctggctcgaactcctggcctcaagcgatctg |
| | | cacacttccgcctcccaaaatgccaggattacaggcgtgagccat |
| | | catgccctacccccccatcaattgtttgatgtagccatttttcaa |
| | | tgatccgcgattaagaagcagcactcttttatagccaaaaattac |
| | | acatatataaaattttcctttagaaaatgttctaaaaatggaatg |
| | | tctaactaaagggttaggcatacattcttaagacttctgatacgc |
| | | gctgacttgcaggaaagttgtttcagttaacactcctaccagcgg |
| | | catccgagagttaatctgtaaagcttgagacaacttagaaagtgt |
| | | ttcaaatgattgtgttgcttaagaaaaaaatcttagcacttcctt |
| | | ttgaaaagccagtggggctgaaaagacaatgacaagcactttgtc |
| | | cctctgtactgtgttttccttgcagCCTGGTGAGACGAGTGGACC |
| | | GGATGGAGCATTCCATCGGCAGCATAGTGTCCAAGATTGACGCCG |
| | | TGATCGTGAAGCTAGAGATTATGGAGCGAGCCAAACTGAAGAGGA |
| | | GGGAGGTGCTGGGAAGGCTGTTGGATGGGGTGGCCGAGgtcagta |
| | | gtcatgagctgaaaacaccgctgctgagcatggtgttattaatga |
| | | aaatatatgttgctgacagttgtatttgaagtattgaagaagagt |
| | | aaaaaaaatttacgtttatagaaattcacaatgatgtttccattt |
| | | actctcattttcagatttttttctctgaaacagaaacactctttc |
| | | tataaaatctcttgctataaaacatcaatgtagtcatattgtcta |
| | | acccttaggctgagatgtttatctttctccataactacagataaa |
| | | attataatctggaggtgttactttcttaatactccatatgctaat |
| | | ggtcctgccttcactgcagggtagaattaagtgaaaaattactcc |
| | | agcaactctgagatttgctattatatgctgtaaatctccagcctt |
| | | accaaactacagattatttggtccctggacttcctaaggcatttc |
| | | cttctactgcccccaacaccagtttctttttccctttttagGATG |
| | | AAAGGCTGGGTCGTGACAGTGAAATCCATAGGGAACAGATGGAAC |
| | | GGCTAGTACGTGAAGAGTTGGAACGCTGGGAATCCGATGATGCAG |
| | | CTTCCCAGATCAGTCATGGTTTAGGCACGCCAGTGGGACTAAATG |
| | | GTCAACCTCGCCCCAGAAGCTCCCGCCCATCTTCCTCCCAATCTA |
| | | CAGAAGGCATGGAAGGTGCAGGTGGAAATGGGAGTTCTAATGTCC |
| | | ACGTATGATATGTGTGTTT (SEQ ID NO: 238) |
| | premrna | GAATGATAGGGGAAAGGAAGGCAAGGGTGAGAGAAGACCTTGT |
| | ENST0000 | GTGAATTTGTCCAAAATGTTTATCCACAGGAACAATCCCTTTGTG |
| | 0506367.1 | AAGGCTGCTGGTATGTGAATGTGTGCCGGTTCCCTTGGGGCGTTC |
| | | ATTTGGATCTTTCTGTGTTCCAGTGACCTACGGCATGATGAGCTC |
| | | CAATGTGTACTACTACACCCGGATGATGTCACAGCTCTTCCTAGA |
| | | CACCCCCGTGTCCAAAACGGAGAAAACTAACTTTAAAACTCTGT |
| | | CTTCCATGGAAGACTTCTGGAAGgtatttgcaaataactttgaaa |
| | | gtacctctctatcacagaaaattgttcatttggcttcatcatttc |
| | | aatgcatgagtatcgacaggacctgctttgcatttaacactgtgt |
| | | gagacgtaagttatggtgagttgttagaagttactgttcctactc |
| | | tcaaagggggtaaactaacattgagaactttgcctgtgccttgca |
| | | ctgtgctgagtgtttcatatcttaccttatttaatttctatagtc |
| | | taactctataaggtaagtactaagactatgccctagtttgttaat |
| | | gaggaaaatgagattcaggatgtttaaatgcgtatggtcacatgg |
| | | ctagggaacaagaaaaattgatttttttctagcctgacagctact |
| | | tcatcctagtttgtaattcattccatgagtcaagattcaataaat |
| | | atttattgagaatctcctagaatgtaaggccaatgaagggcagtg |
| | | tggttcttctgtcttgcttcgccttttgtgttttgtctctttgtt |
| | | gatgatggcatgtatccccagctcttagaacagtgcttgattcaa |
| | | agtaagcacattctttcaaaggtctgctgttggtggggcttggtg |
| | | gctcacgcctgtaatcccagcactttgggaggccaaggcaggagg |
| | | attgctttagcccaggattttgaaaccagctgggcacaacatagt |
| | | atgactttgtctctccaaaaaagttaaagaattagcagggtgtgg |
| | | tggtacacacctgcagtcccagctactcaggaggctgaggtggga |
| | | gaatcacttgagcgattgcttgaggtcaaggctgcagtgagccat |
| | | ggccatgctactgcattccagctggggcaacagagtgagaccctt |
| | | tctcaaaaaaaatcccccccaaaaaaaaacccaaaaacaaacaaa |
| | | aaaggtctgctgttgtgaagttcaacccaatccagccccttccca |
| | | agttgtcacaaattccaacgtagttaacagtataccaatgagtga |
| | | taccacaggaaaaatattaaactgatctgagggatatggggcttg |
| | | gaatctaagaaaattggaagggaaattgaaaaggaaattattatt |
| | | tctccttggggagatagtttctaaaattcttactacaccctgggg |
| | | tcagagctgttgattttaaggatagagacaactgagtcacaggaa |
| | | actattcatatataaaagtacctggcatccaaaaccacacttgta |
| | | taatatgaatctttcaccatctgagtagggcaaatcagtctatct |
| | | ctgttgatcatctgacaaggatagcacactgagaaatagatctgt |
| | | cttccctacaggcatagctagttgtacaaactaacaagagacttt |
| | | tgtatacacattccatgatgataaatgccaatcactaaagggacg |
| | | aggagggattggagagttcaccatacagcaaaatagtccagacag |
| | | gtgaaaggtctatcaaatgccaggctggtaatcaaaactgtagcc |
| | | ttttctctaaacaaagtttagaaccatgattgtgtgggacattat |
| | | tttaataagggaaagtgcagttaatcatgaccccacctttagtcc |
| | | aagaacaaaaatcagagctgccacgtattaagtacccactctgtg |
| | | ccaggtgcagtaactatgcaaaagatgggttttccagatgcaaga |
| | | accttggttcagaggaccctgctcaaggcctcatagctaacaaat |
| | | gatggggcaagatgctatcccaaatctctctgacaacaaaactca |
| | | ttcttatcactctactattttcatagagttgccaaatgcttggtt |
| | | atgcaaacgatgcaggcaggggcaagacagcggctgagcttggaa |
| | | ctttttcagagatgtttcctttgcttttagTTCACAGAAGGCTCC |
| | | TTATTGGATGGGCTGTACTGGAAGATGCAGCCCAGCAACCAGACT |
| | | GAAGCTGACAACCGAAGTTTCATCTTCTATGAGAACCTGCTGTTA |
| | | GGGGTTCCACGAATACGGCAACTCCGAGTCAGAAATGGATCCTGC |
| | | TCTATCCCCCAGGACTTGAGAGATGAAATTAAAGAGTGCTATGAT |
| | | GTCTACTCTGTCAGTAGTGAAGATAGGGCTCCCTTTGGGCCCCGA |
| | | AATGGAACCGCgtaagtgtctgtgactcattgccactcggtgata |
| | | ttcattcatttattctctgaactcccaccattcattcattcattc |
| | | cctgacaccttcaccaaggcaaaaataagttcagtgactcttcag |
| | | tgcttatatttaaaccttggccaacttgacctttgacttcttaag |
| | | ttttcactacttcttagccttcttttagtttctacatgcatattt |
| | | ttcagaagactaaatcgttgaccatataacccctcaaaaattaat |
| | | tatctgagcgtttgaaaatttcatttaagatgccctgggccctgt |
| | | ttttacaggtgcagtaacatcatccactaagttatttaacacaag |
| | | ttttctggttcaggaactctttttataggtcttgcaaacaggttt |
| | | ttgttcagaatggagttatttaatgtgtaagcttgtgaggcaatt |
| | | ttttgttaggtttaaagcccattttgttcaaatgtttgagatttt |
| | | aggtatatatttgtacacgtgcatatttacaggggctttttgtac |
| | | actttggtactcctacttcaaacatcttgtgtattaagggaggtc |
| | | acttactattttagaagtattgtagttattataaagaaacaagaa |
| | | gacctccaaggccgtttcagggtgggcctttgcggttgctgtccc |
| | | tgggtacgtcactggtcggagtcatcttctaagctttgctcagct |
| | | aattctgtcggttcatctaggttcttttcttggaaactgagttgc |
| | | ccagaatccacatttgttactatacaatgggcaatcaccttttca |
| | | attagtatattcttcttgtaccttccagtatacactctatttaat |
| | | accagaacccataagaaacaaatttagtaaaaatccaggttgggc |
| | | acagtttctcatgcctgtaatcccagcactttggaaagccaaggc |
| | | ggacagatcacttgaggtcaggagttcaagaccagcctagccaac |
| | | atggtgaaaccctgactctactaaaaatacaaaaattagctgggt |
| | | gtggtgacatgtgcctatagtcacagctattcgggaggctgaggc |
| | | aggagaattgcttgagcccgagtggtgggggttgcagtgagctga |
| | | ggtctcatcattgcactccagcctgggcaacagagcaagactccc |
| | | cctcaaaaaaaaaaaaaaacaaatttagtgaaaatccagagcttt |
| | | agaacaaaggaactaaatagtctcaaaggacattatcatccaagt |
| | | tatgatagtgatttcgctttctttaaaaaaaaaattattacagat |
| | | agagtttcttgatgttgcccaggctggcctcaaactcctgggctc |
| | | aagcagtcctccagcctcagcctcccaagtagctgggactatgag |
| | | aatatgccaccatgcccagctttattttgctttctaatgtgcctt |
| | | tttgtagttcctgcaaagcataagcatgccttcatctgtggtacc |
| | | ctttccaatattttatttatctcacatcactaataagataaattt |
| | | atacagccactgctctgtgccagacattatttaagaagttatttc |
| | | acgcattatctcatctgccttcacaaaacaactctaaaataggta |
| | | tcacctccattttatagatgaaaaaactgaggctcacttgcccaa |
| | | agtgtcacagctaacaaattggactgaaccaagatttaagcagcc |
| | | tgactccaaaacccatgtttcgcctactaaacctcttccatatta |
| | | attcctcctccatattaattgcgtcgttagggtggcttgtcgacg |
| | | ctctcagctccccatcagtactcaagcttcctgagggcagggatt |
| | | ctattttgttaactgctgtattctcaaagccttgaacaatgcctc |
| | | atatgtaaagatactaataaatatgtgctggatgcattcgagtga |
| | | tttctactccagggtggattgccaaaggggaccttccctgtcata |
| | | tctaaactatccttttattcttctacattctcaagtcactcccat |
| | | ttcttcttcccttcaactccaaacttcttggaagaatagtcttca |
| | | tttgccacttccatttccttaccatcaattcacgtcttaaagctt |
| | | aggttcttgcttttgacatctcaccaggaagctactagggacctc |
| | | tagttagcaaatccactgaacaaatctcagtttttatccccttca |
| | | tactgtctgcagaaagtggcagtgttgaccgctcattcttttgac |
| | | ttccatgaagaactctacactttgggttcttttaactctgaaacc |
| | | cctttctctcacttctcttattcccaggttctatattcatatctc |
| | | cgtcttttctttcttttctctctttcccatgacattcagatggct |
| | | tccactgacatctttatttggtgactcatcatttcttcaacaatg |
| | | tgccaggcatgttcccattgctagaagtcccccctcttctagtag |
| | | tgcccttgatgctggagaaacaaagcagcaggaaacagaagaaac |
| | | gtcctttcttcacagagcttgcattctagtgatacaagacacatc |
| | | tctattccacctggatgtcccacacctcttgtgatttaatatgcc |
| | | ccaaactaaactcatcgttttcttatgagatctgctctttgttct |
| | | gttaatggcactgccgtcttcctattgccctgaaagagactccag |
| | | agtcatttttgagccatgtttcctccttgccccaacttccaaatc |
| | | agttaccaagttctgctggcagtcactgtggttacgacactctct |
| | | cactccttcctttcccttcccatcttcactgagtgacttcaggcc |
| | | tcattacctcttggttattgcagactttgctaagggagctgtgtc |
| | | aggggtccctaagaccacccccaggttcagtgattcactagaagg |
| | | actcagcatagaggcatccttacagctaaggtttattatggtgaa |
| | | aggatacaaaagaaaaggcacatggcaatatctggggaaaacctg |
| | | gtgtaggcttccaggagccctctcccagtgaggtcacataggatg |
| | | tgctgaatttctccaggaacgagttgtagcaacatgtgtgaaatg |
| | | tctatcagagagattaattagagactcagtgcctggggtgttcac |
| | | tgggtactggtactggtttatgactagcatgtgccaaaattcccg |
| | | actctcagaaggaaagcaggtgttgagtgtaagccacattgctta |
| | | tacaaatagcacaggcacagcaagcctctcttaccggtcagggaa |
| | | agttgaatactggtgcagggagctgtttatcagtcaagttcccag |
| | | atgccagccaaagtccaaccttgcaaacaggccttcctaaagaaa |
| | | ggccatctcaggcctgctgtgttaactcttttctgcacatgagcc |
| | | ctctgttcctccccactccagtcttccctgctgccaccagaccag |
| | | ccctgttgtgtgcctcttagaactttcccttcacagactccttca |
| | | tgtttgcccttcacaagctgaccacagaccacctttcaatctgcc |
| | | attctttatcttatgggagccaaactgaactacttcttctcttga |
| | | ccaatggctcacggttgaaaatgcccttcccatttgtattagtct |
| | | ggtctcatgctgctgataaagacatacctgagactgggcaattta |
| | | ccaaagaaagaggtttaatggacttacagttccacatggctgggg |
| | | aggcctcacaatcatggtggaaggcgaggagcaagtcacgtctta |
| | | cgtggatggcagcaggcaaagagagagagcttgtgcagggaaaat |
| | | cccacttataaaaccatcagatctcgtgagacttactcactatca |
| | | tgagaccagcatgggaaagacctgcccccatgattcaattacctc |
| | | ctaccgggtttctccaacaaaacatgggaattgtgggagttacaa |
| | | ttcaagatgagtttggttggggacacagccaacccatatgaccat |
| | | tcacctgtgcaagtccagatcctactcacctttcatggttcaggt |
| | | ccaacacagcttcttcctaaagccttttctaatccatccctccac |
| | | tgccagatccccaaataaggacagctttcctctgatctgccagcc |
| | | ttatttcttttctcttttgcttcttattattatccaccttgtatt |
| | | ttacttgtttaattataactcttatttccctacttgttataaatt |
| | | tgggagggaaaggacctttatatccccattagtgctgaagaagca |
| | | tctgttttctaagagatcctctgaatatttgtggatgaaattccc |
| | | aaatgctgacaatgccacttgatgaacttgtccctcttttggctg |
| | | taattttctctggggacctcaagtcgtctagtccctgagcacagt |
| | | tacccagagaaggcagccgtataaactgagcagaggctctagagc |
| | | agagcagaggctctagggacaaacagccctggcattggttcccga |
| | | gtctgccctcaaaagctttgggactacattgacctcactaagcct |
| | | cagggccctcattcctaaagaggctaataacagtatgagaattaa |
| | | ccaagaaaacatgaactgccaggtcaggcacagtacccagcttga |
| | | taggccttaatacatactttattttacaaggaaccagctgtcctt |
| | | gtaattgcctcaagtgttccactgattgtaactgtttgttttttg |
| | | gttttgtttttaatcagTTGGATCTACACAAGTGAAAAAGACTTG |
| | | AATGGTAGTAGCCACTGGGGAATCATTGCAACTTATAGTGGAGCT |
| | | GGCTATTATCTGGATTTGTCAAGAACAAGAGAGGAAACAGCTGCA |
| | | CAAGTTGCTAGCCTCAAGAAAAATGTCTGGCTGG |
| | | (SEQ ID NO: 239) |
| | premrna | CCAGTCGGCGGCGGGGACCCGCTGCATCGCCACCTCCCCCTGGAA |
| | ENST0000 | GGGCAGCCGCCCCGAGTGGCCTGGGCGGAGAGGCTGGTTCGCGGG |
| | 0506727.1 | CTGCGAGgtaagagcgcgcgacccgcagcggcagatgcacgaacc |
| | | agaacggccggcgccggccggggccatcgcccgctgcggcagctc |
| | | cccgggctccatctcgcatcccctctgcgttccgcctcccttgga |
| | | agcgcattccccacctccgctagtgctgccctatttccggtaccc |
| | | agcgcggaattccactgctcttttgttggtgcatatttattggat |
| | | acctccttcttcaggatatgtcaccatagtcttttttactgaaaa |
| | | ttagtgaaagcctaattagagtgaaagagtacatctgggttttgt |
| | | ttttttttttcttgtagaggaaaaaatgaacattacttgtgtaac |
| | | tgatggtagttgcaactgcatatttgccaatgtcacaaaatctaa |
| | | aggaaaatgttatagtcacccgtggtttccttcttgcctggacac |
| | | tccattgtcccgggctgaaaagggtagcagtacagtgcatataat |
| | | gtcaagttgtgggaggagtgtggcagattgtcattggtgcatttt |
| | | tttggtgatgtgtgtggttgttttgaggagtgggagctgttaaga |
| | | acaccacaaatagaataaaataatatcgtgaagttatttggccgt |
| | | ttctaattctagacatttttctaaaaacagttgcaaaggaaagat |
| | | tacattgttttaaaaaaatttgaagtatgtttttaaataactaaa |
| | | ttaatgttctttgaaattccaccaaaatgatgaagtcaccagata |
| | | gcagctgataaatgtgtctgagcccagtgcgcccagctctacaaa |
| | | aggcagaagaggaattttcaaatttgccagtgcccagtaagagga |
| | | catgaattttctagtaccagaggaaattctctttttacaaatttt |
| | | gtcagaggtatccttgggaaagtatttcatttgcttttaccctcc |
| | | aaattattttaatctatatttttaagagtttccattctcagttga |
| | | gtttttcttgttcttctccttctgtcagttttgaaagtcttgcac |
| | | aaaaacaatccaggtgttgttacagcagtgtgattaaaaccaggt |
| | | caggcctacactgaaatcccagttccaccactcattagctgtacg |
| | | accttgagcaagttacttatcctctctgatacccaatttttggat |
| | | tttttttttttttaaagagatgatcataacgcttaggacttgact |
| | | ctttgggagaattaagtgagttaagacataaaatgtgcagcatgt |
| | | atttgtcatgttattagcgcttcagaaatataaatgtaaacattt |
| | | tggtacttcgtttatggagattcttatactagttaattttattaa |
| | | aagcatgatggggaacagaagatccttttcggataacctgtgtga |
| | | gtaaattaataaaacactaacactttttaagattcaaaactggat |
| | | taattatgttattttacaccatttaaaatgtgcatttaaaaaata |
| | | ttcactgaagtgagagagaagttccttttaagttaataaataatg |
| | | gtaagttgcatgatccttttaactcagtttaagcatttgataaca |
| | | cccctaaccttgtttgaataaactcaataacagaatatagagaaa |
| | | taaaatatatatttcatatgagtatgtatgtaaatttatttcttt |
| | | taaaggaaaatttcaggaaacaaaatgaataagctcatagtcata |
| | | aaacctctagccaaagtgtgtcaatggtctgatttgtaggagagc |
| | | agactcaggaatcgggaattttttttttttttttaagagtcgggg |
| | | tttcactctgtcactgaggctggagtgcggggaagcaaacgtagc |
| | | tcactgcggcctcaaactcctgggctcaagctgccctcccacctc |
| | | agcttcccaagtagttgggattacaggtgcaagctgctgctccta |
| | | gctgaaaagttttaattatataaaatgtttaaataaattgttatt |
| | | cctttcttttatgaagaaatataattgatattcttgtcacttatt |
| | | aaataagcaacattttaaaatgtttagcacctactgtgaagatag |
| | | cactgtgctagctgctatgaataaactagaatagcatatcactgt |
| | | tttcctatggacaaaaacccagaaatgtaaaataaatagccatga |
| | | atgggttgagattccccctgccccctttggatactgatgacagaa |
| | | atcttatatgcactatgtaagcagtgccctaggatcagaacagaa |
| | | agtaacttgcaatttttaaaggtgggagaaatgactgaagtttgg |
| | | agtggtcaggagtttccctggaagtctcttgcctttgtgactttg |
| | | tatagctccctcaaactacctgagcctgagctcctcatttataaa |
| | | atggaagaattgaacctagcttccaaggtcctttctagcttcacc |
| | | atgctctggtctgttgtttaatgacatgaatccaagatggacaac |
| | | aaatggtcgattttgctccttctacagtaaatagatctatcttct |
| | | tagcagaagtaaaatagtaaagaaagaagacatgtttgaggcctg |
| | | ttgaggcctgtttgctgtatgcattgtatctaatcaaggaattga |
| | | gaatgtagccctaaatattaggaaggagttgaaagtttctgagca |
| | | ggaacagaacatgctgaaaggaaattgtgtcagcagcatggtgta |
| | | caggagagcttggcagagagaaaggaggcaagaagacctgtggga |
| | | ggcagccagtaaggaggtgaagagggcctggaccaaaggaagcca |
| | | aggatgaccgaaagatttaaagatgaagcccagatttaaaagtat |
| | | cctcaagtatttgtttattcttttcttagcaaattctttttagta |
| | | caaagataaaatatggcactctgagtcataaaattttctgaattt |
| | | cagaagttgaatgtttttctgaatgtatcaacctgttaaagtcag |
| | | ttcctgtttgttattttaggcgtatattcttgggctttttttttt |
| | | ttttcctagagaaactatgaagtactagctgtgcaagtcagggtg |
| | | ggctaagctgctaaaacagatacctccctccccttcaataccctg |
| | | aagaacaagaagttaattttttgctcgtgtaacattttagagagg |
| | | gtattccaggttgaatacctggaataatgaatgaaataataagtc |
| | | gactcttatttctcctaaaataattggatcttgacattgaaattg |
| | | gtgtgctgattgtatttaataagtgacctccaatgcagttatttc |
| | | attttgccatactttatgtaatttttattttttctgccttccctt |
| | | tcgatgtctaaagggaagcataattagttattggaaaggttattg |
| | | agacttaaaaaaagatttgaacatggctttggttgatgctaccac |
| | | acagaaactgcaaacatccaactcaccgctcttagttgctgttca |
| | | tgatagtctatgcacagtgactgatgaatttagcctgtcttgagg |
| | | cttcagaacttagtcttatgtcatcacgagcagagctttatccta |
| | | aattacaggttttcaatgggggggggggggaggggcagttttgcc |
| | | cctcaggggacatttggcaatctctggagacatttttgattgtca |
| | | tacctggggggatgcccctggcatctcctgggttgaggttaagga |
| | | tgctgctaatctacaatgcacagtacagccaccccacacacacac |
| | | actacaaagcattctcccacccaaattgccaggagtgaggacttt |
| | | gagaaaccctgccctagactgttaaattcaaaaggaaataatggt |
| | | ttattgctcacagtgtgccaggcactgtgttaaactccttatatt |
| | | catagttttgtttttatcctcacaacaacctgtgaagaaagaact |
| | | ctcatccgtcaccactttactgttgagggcactaagccttctaaa |
| | | ggttaaatgacttgcccagggccgcaactattagatgttgcagtc |
| | | aggatttaattccaggcactttgtgttcaaagtgtttctcatcca |
| | | ctgtgctatatgccagtagtgcccaaacctaactttagccagcaa |
| | | ttgtctgcatctcttcagtttatgaacatttatttattaggacat |
| | | gcaggataatcataccaacacagtccgtgtatccagaattctaat |
| | | tgatctagggagtgggagagcctgcctctctattttttttaaatt |
| | | ggtgtgaaatatacataacataaaattggccattttaaccatttt |
| | | aagtatataattgagtgacattaggtagatttattatattatgta |
| | | aatggacttaacacaatttatttccagaacttttttatcatccca |
| | | aacagaaactctgtactcattaaacagtaactccctgtttcccca |
| | | ctcccccttgccccagacgctagtaacctccattctacttcccct |
| | | ttctgtgagtctccctgttgtagctacctgatgtaagtgaaatca |
| | | gaccatgtttgatcttttgtgtctggcttattcacttagcatagt |
| | | attttcaaggtttatccatgcgtgcaaattcccttcttttttatg |
| | | acgaaatactatttaattgtgtgtgtacatgtgcacacacaatga |
| | | aatactatttcatcataaaaaggaagtttgtacacatatatacac |
| | | acacaccaaattttgtttatccattcatcagttcatgggcatttg |
| | | cggtttttttccacctttcgactgttgtgactttccgtttatttt |
| | | taatctgaactttactccatcacttcctccctttccttttttatt |
| | | cgcaccataattttgaacaagcagactttgtattttcattactct |
| | | ggggatttttttgagggaggcttgctaccctttggggttgtggta |
| | | aggttgtgccagtaacagaattcatgcagtaaaaaacattcatgg |
| | | gactttcttttgtgataggtactagggatgcagagatgaataata |
| | | caaggtctcgaccttcaaggagctcagggtttagtagggaaaaca |
| | | gaggtataagtaagtcattgcaatgccaggtcccagatatgggag |
| | | gattccagcaaagagaggataaggccaggcacagccgctcatgcc |
| | | tatagtcccaacactttgggaagctgagatgggaggatcacttaa |
| | | gcccagcagttcaaggctaccctgggcaacatagtgagtggcaaa |
| | | aaatacaaaaattagccaggcagggtggcacatgcctgtggtccc |
| | | agttatttgggagtttgaggtgggagaatcacctgagcctgggag |
| | | attgaggctgcagtgagctgtgatcacgccactgcactccagcct |
| | | gggtgacacagtgagaggtgagagcctgtatctaaaaaaaaaaaa |
| | | aagagaggatcagatttacctggagaggtcagagaaggtttccag |
| | | agggagtaaaacttgagcttcattttgaagaatgagaggggaata |
| | | caaggagggaaaaataataggaacaagacctgttcctgttacagc |
| | | aaacctgtactcccaccatggaatgggcacgttttcctgtaagac |
| | | atttggagccagtgtgggcttcaagcctagggggctgatatgatc |
| | | atttgtcaatctgaagatgactcttggaacaacgtagaggatagg |
| | | gtgaggtgggtaggctggtggctgacagactaggtcaggagaggg |
| | | ctgaccaagaggaggaggggtgacgagggctggaactaaggcata |
| | | tgagctgggatggaaagaagctagatagaaaggaaacaaaaacca |
| | | tcagaactgtggaaccaactccatgggagggataagaagagggcc |
| | | tattctagggtgaaacccaaatttctggcatggtaccattaaccc |
| | | aaacagaaagaggagatttccagagaaagaaaagcaaattttgcg |
| | | ggaggtgagtgtgagctgtctagagaatatgcaggtagaactaca |
| | | tgttaactgtaacatatacttgtctgcacctcaggagatggctga |
| | | ttaagaattcaggattcaggccaggcacggtggcacacgccttta |
| | | atcccagctgcttaagagactgggggggaggattgcttgagcctg |
| | | ggaatccgagatcagcctgggcaacatagcaagacactgtctcta |
| | | aaaaaaaaaaaaaattcaagattctggagtcaatattacttaagg |
| | | tagcaactgtctattctatagaaaatggacaaatatagataaaaa |
| | | gccacttccctttctaaaagtgtccctgcaattcaagtgaatact |
| | | aggaaggattgtgttcattcttctaaacaagactcacatgtatct |
| | | tagcacaaaaagaggattcttttatgatacaaatgcactgagaat |
| | | ttggtcaggctatcacaatgaactgatagttcagatggatttgag |
| | | tctttataccactctggaatctggaccaactgggcctcctaaggc |
| | | cattttgcagatctgggcttgtttctgaagctacagacaggcctc |
| | | ttccaagcactctaagtgctccacaaataatatttctgtttccca |
| | | gaacaaccccacaaaaaggtactcttactccatttttagatgagg |
| | | aagtggaggctcatgatgtcaggtaagctttctcagctcccaagt |
| | | ggttaagccctcagtttaatgtcatttgactccagagccctatgt |
| | | tgcaccatgccttgataataggccatatgggtttcatgtatttca |
| | | gatggggaaggttagtgtgaggtgaaagatacacaattaaccttt |
| | | taaccatggaactgaaatatttacagatgaagtgatacaatagct |
| | | ggaattaattccaaaataattggggtggtactggtccatggcctg |
| | | ttaggaactgggccgcagagcaggaggtgagcagcaggctagtga |
| | | gcattgctaccccctatcatatcagcagtggcatcagatgggtga |
| | | ggatgtaaatgaactaggattggcattgagttgatattgttggat |
| | | ctaggtgatagatttatcattataatattctctctttgtgatatt |
| | | tctgatattttctaaaataaaaagttgtagttatttatttattta |
| | | gacggagtttcactcttttgcccaggttggagtgcagtggtgtga |
| | | tctcggctcactgtagcctccgcctcccgggttcaagcgattctc |
| | | ctgcctcagcctcccgagtagctgggactacagcctcccgagtag |
| | | ctcaccaccacacccagctaacttttgtatttttagtagagagga |
| | | ggtttcaccatgttagataggctggtctcaaactcctgacctcag |
| | | gtgatccacccacctcggcctcccagagtgctgggattacaggcg |
| | | tgagccagtgtgcccagccaataaaaagtttttgaaaggatttag |
| | | ataaaatagttggggaaatggcatttttgttcaaagccaattatt |
| | | tatgtttggaatatcttttgtgcttggagttctccattacagagt |
| | | tccccagtgttcctattaataagtaacattgagcagaggaatgca |
| | | ctgtttagatcagcagtccccaacctttttggcaccagggaccgg |
| | | cttcatggaagacaatttttccacagacctggggtaggaagtgtt |
| | | tggggatgaaactgttccacctcagatcatcaggcattagtgaga |
| | | ttctcataaggagcaggcaatctagattcttcacatgcgcagttc |
| | | acaatagggttctcactcctgtgagaatctaatgccacccctgat |
| | | ctgacaggaggcggagctcgggcggtaatgctcacttgcctgcgg |
| | | ctcacctcctgctgtggggcccggttcctaacaggccatggacca |
| | | gtacccgtctgcagcctggggactggggaccctgctttagatgat |
| | | gtactctggctttgcattctggcattagctaagcaccctcttaaa |
| | | ggaaattgggtctatactctcagtccgtgttctccctaacacctg |
| | | gaaacattgaataccttcaatgctgggaagttaactcccaccaca |
| | | actagaacagctatgggaaagacaacagttgattttgaagagtgt |
| | | caccaatttgcacatgattccatccttaaccattcttatcctatc |
| | | agctctgccaaacatggagaatagttggctgcaggacagctattt |
| | | ttcctacttgtagatgcaactatttctcacccaccaggatgtaaa |
| | | aggtccctgtaccctaagattggtcctacatacacacccaatggg |
| | | aaaatgagatgaaaaatttaaagcagtaaatatttgaggaagtag |
| | | atagagtaatttagaaaaagaaaatacacagggccaagcacagtg |
| | | gctcacatctgtaatcccagcactttgagaggccaaggtgagagg |
| | | attgcttgagctcaggagtttgaggccagcctaggcaatgtagtg |
| | | agaccccacctctacaaaaaattaaaaacttagttgggcttggta |
| | | gtatgtacctgtagtttaagaaacttgggaggctgagctgaggca |
| | | ggaggattgtttgagcccgggtggtcaaggctgcagtgggccatg |
| | | attgtgccagagtactccagcctgggtgatagagtgagactctgt |
| | | ctcaaacaaaaaaaaaaacagagacagaaaaaaagaaagaaaata |
| | | tatggatgtatatcatataaaaatataaataagggaggccaagtg |
| | | cagtggcatgcctgtaatcccagcactttgggaggctgaagcagg |
| | | aggatcacttgaggccgagaattcgagaccagcctgggcaacgta |
| | | ttgagacctcatctctgcaaaaaatcaaaaaatgaggcggaagga |
| | | tggcttgagcccaggagatcaagccttcagtgagctgtgatcgta |
| | | ccactacactccaggctgggtaacagagagagaccctgcctcaag |
| | | ataaataaattcatacatacatacatacatacgtacatacataca |
| | | tacataagaagacttgtttctttccatttgcaatgtttcattcaa |
| | | aggctagaattaaattgccgtaggccatcacaagtttagcttgaa |
| | | tattattattttttcaagatggagtctcactctgtcacccaggct |
| | | ggagtgcagtggtgtgattttggctcactgcaacctctgtctccc |
| | | gggttcaaccgattcttctgcctcagcctcccatgtagctgggat |
| | | tacaggcgcccgccaccacacctggctaagttttgtatttttagt |
| | | agagacagggtttcaccatgttggccagactggtcttgaactcct |
| | | gacctcaagtgatctacccgcctcaccctcccaaagtgctgggat |
| | | tataggcttgagccactgcacccagcctagcttgaagaaaatttg |
| | | ataggagtttgtttttttctatttataggccaagcaataccacgt |
| | | ataaatattaagaatcatggctgttccttagtgcctagttgttta |
| | | taaaccatgggaaagaatgaaatcattgaccaaatgagacagggt |
| | | aacagtcttccctgggagtaaagagactagcagtctctattgaca |
| | | tatattttaggcctggcctccaaaataaatttacccaaagaagtg |
| | | atgtatttgtgtccagggctaccgagcctaatctttggctgcctc |
| | | tgtgttattctaagttgtaattttccatgtcatctaaatttgtaa |
| | | taattctttaatatgatagtgtttcagtgaacaaacattctgctt |
| | | gctgcatttcttctgagtgagtaattccctgacaactaccagatc |
| | | ttggcagaagcaaagttggtcataagttatgctccactctcagtg |
| | | ctggtgaaacgtatgatgcgtaacacagtgtttttgaattcagtg |
| | | ctgattttcctaaaggacatttggaaggaaaaaagaaaaggaaag |
| | | gaaatacccaaattcaggaatagaacttacatattattataagac |
| | | ttaaaaaatacatgaacactaatgatgaactcatttcttcgaagt |
| | | aaaaggccttatgctattttttcccatttccctatgtggcttgat |
| | | tgtggcgaaagtggctgtgtgagtttccattattgaaggagttaa |
| | | ggtctgtggaatcaaagcatgagacaacatgcaaggaccaggttg |
| | | gtttccatttaacagccaacctaatctaactgaaaggattgagag |
| | | gtttgtcttttttggaaagtgttaaggttcttccaagtaaccagc |
| | | aatgtgactttaccacactgttcattaacggggctttggatggcc |
| | | accatgtctgctgctggctgagtccaaaactgcggtcactctctc |
| | | cagcaagctcatggagggttacgtcatcttcactcagccagccca |
| | | acgctttttccatctgctaaaatgtagaacatggttgttgattct |
| | | actctttctacaaagaaagagagggaacatggatgagttgctgct |
| | | tttaaagattatatgttaattgctgttttaaaaatctgctcagct |
| | | aagcacgcttagtgtaatcagtcaccatggagttttttaatagga |
| | | cagcttctgctcttacagagcagagtttttatgccagtaggttag |
| | | aagcataacatgtttctatattgaagtgattctccaacaaggact |
| | | tcttttcatcaggggtgacctaaaagttattacttgaaatataat |
| | | tacatcatgttcttaactgggtttagtagtaggataattacaagg |
| | | ttgtatccacctcaaataggaagaactgataagttttgcacaatt |
| | | atttaactcctctgttaggccttccttatttcctgttctcttttt |
| | | aaaatttgactaagatattgctaatgggcttgggagcctataaaa |
| | | tgatcagaattgttgccttatgttttgcatgtttggggtaacatt |
| | | ggagccagatgtactcttaaataaaaggtaggcctacaaaaccag |
| | | tttctcagttgcattcaaaatgtgattaaaaaaaaaaaaaggaga |
| | | atctcccttataggtgaactttttaatttgtgctttattttccct |
| | | tttgccattcatgagatttcttaaataaaatgatatcttttcttt |
| | | tcttcattattattttaaagGTCTCTGGGGAACAAGACTCATGGA |
| | | GGAAAGCAGCACTAACCGAGAGAAATACCTTAAAAGTGTTTTACG |
| | | GGAACTGGTCACATACCTCCTTTTTCTCATAGTCTTGTGCATCTg |
| | | taagtagaatatttccttgcactaatgggaaagttttgaaaagat |
| | | ttgacctatccaaatcataattaaaaggaagtgtgtatgcaccag |
| | | aggggcaactgggaagttaccttcttacctttgtttttaattcta |
| | | atatttttatttgggcatttgtttattgactatcttcctatggta |
| | | gaatgcaagctttataagagaagggacgtgatttgttctctgctg |
| | | tacccccatttcccaaaactgcagatggcaacagaaggctctgaa |
| | | aaatatataagaaagaatttttctaattgtgactaaattgtgacc |
| | | aaatgctaagtgactgtggacttgcgtttaacacaggacgggaga |
| | | ggcaaagagttcaattccaatttagaatttggtcaagttctcttc |
| | | tgcactctggtaaacattaattaaaaatcagcattatctgaccag |
| | | ccagttcatcgtcagtggtggtgattttcactatgagatacgcgt |
| | | ggcaacttgccagacaccaagaaaccaagttagaggatttttgta |
| | | ttagattccttaacaatgaatacagtatcaccattattacagtat |
| | | catcattattgtcatactattattatatcagttaacataaagtct |
| | | gcataagaattgtttccagaaaaatgactttccaaatttaacttt |
| | | caggaaatacaaataatgctactaatattgcttttattggcgtat |
| | | acatgtaatatccccttcttttggatttggatatgttgtgtcatt |
| | | gcctcattttaattcattatttcttctcaatctttaataattgct |
| | | ggacttttactccacaagaaacttgctataggcccatctctttcg |
| | | tcttctttccttctttcagttcgtcttcccatcctctggtagggg |
| | | gaggggagggatgcctgagcgagagactagctgtaggaaccattt |
| | | gtctcaaagtccagaaagccacaggtgatggatttgtcctctgaa |
| | | tcaaagggcgttcgatgatggatttctgtcatgtctcatctaaag |
| | | tcttcacgagaacagatgaggaagcagttttatgaccccagagcc |
| | | tcctaccaaactcctctgagaaaaggtttcctttttttttttttt |
| | | ttttaaattagagacagagtcttgatctgtttcacaggccggagt |
| | | gcagtggcacaatcatagctcactgcagcctcgagctcctaggct |
| | | tcagtgatcctcccacctcagtcacccacgtagctgggactacag |
| | | ctgcacaccaccatgcccagctaacttttaaaacatttttgtaga |
| | | ggtggggtctcactttgctgcctatactggtctccagctcctagc |
| | | ttcaagtgatcctcctgccttggaagttctgggattataggcatg |
| | | agccactgcacccagcctggatgtgatatttttatgttttaaatt |
| | | gttagagtttagaaacttgagattgagtttgctgcctgcattaaa |
| | | atgatgcttaaacattaaactgcagtggccttaaatattaacaag |
| | | ttgattagaattactaagttcttttcaagctttacatatacagac |
| | | aaatttcttatgcaaaatagaaggtaacccctgtacgtaagtcta |
| | | gaatttcagcagtccccaaaactgactgagcattagaatcactct |
| | | gattttaaaatacatatgtggttttccgagatctactaacagagc |
| | | ctccataatgtagcctagagaaacgagttttcagatgtagtgata |
| | | aatttggaaggtaagtcagagaagtaagctgaagacagagtttta |
| | | ggaaatatgcctaaagtcacataatgaattggttttcttgtttat |
| | | ttgagaatattgtcgctttttgttctttttcatgcaaatcacatt |
| | | ttatttcttatgtgagtagctatatatttaaaaattttgtttttg |
| | | gaatattgtagaatctctacttaagaaagtatcttagcagtcata |
| | | tggtctgacctcactgaatgctaaattctctttaaaacatccgtc |
| | | tcagatggttactcatactgcctctgattgaacaagagtcccagg |
| | | ttaagggacttacttctttgaaatcgttcatttcatttttctaca |
| | | gctatgttagaaagttcgtccttagcagtgaagccagagtctatc |
| | | tcttataacttctacccagttgcaccctccaagcctacctatacc |
| | | aagtatcttttttccacgttgcttttccaattcagctcttcgcaa |
| | | gtttggagacaaatacctaatctcctctaagccttctccaggtta |
| | | agcctttccagttcatccagctgttgattatgtgattggagacac |
| | | aagtttgagtaactcccatgatggaaagtccctctggtgaatgtt |
| | | ctgttcatcagagtccctaagaaagcacatgagcctcaccatggt |
| | | gagtggggccatgagattcctaaaccagacactacactgtggctt |
| | | gtgcaacctaccatggccagactcatgaggctgtttcatcatata |
| | | aataattccttagtctcttcaccaaaaactgtgaagcactgtgtc |
| | | ccccagctgtatgtgagctagcctgggaccatagtggaggactcc |
| | | tctttcaaccctgttaaatttcatcttgttaggatctgcccattt |
| | | ttccattctgttgaaattatcttggaactggattcattcatctca |
| | | catcagctactccccgaagcctcacagtatcagcagagttattat |
| | | ctctatccatatccttgtaaattattaaactaaaaaagattggtc |
| | | taagaacatcagtccattaatcaaggctctttggttggagttcac |
| | | ttcattatattatcatccagctcagagtgttcataaacaagatga |
| | | taggaaagacttttccaaatgcctgtctgagtaattcccccattc |
| | | ctgtgatctgtgagctgttgaccagattaaaaaggaagtgagaat |
| | | aaccaggcaagatttactcctagcaaaaccttactggcttctagt |
| | | gactgagtcctttccctattgcttaccagctatctttttaattac |
| | | tagttttaaaatcttgccagcaatgtcaatatcaaatgaccaaga |
| | | atatcaaactcattactctgtatgtaaatgagatagtgtacttac |
| | | ccctatggcttaagtattaggctgttcttgcattgccctaaatac |
| | | ctgagactgggtaatttataaaaaaagaggtttggccaggcactg |
| | | tggatcaggcctgtaatctcagcactttgtcaggctgaagcaggt |
| | | gtaatggtgagccaagagttcaaacttagcctggacaacaaggtg |
| | | aaaccccctctctgcaaaaaatacaaaaattatctgggcatggtg |
| | | gcatgcacctgtagtcccagccacccaggaggctgaggtgggaaa |
| | | attgtttgaagctgggagtcagtgattgcagtgagccatgattgc |
| | | aacactgcactccatccagcctgggcgacagagcaagaccctgtc |
| | | tcaaaaaataaataaatgaataaataaataaaataaataaataaa |
| | | tagaaaagaaaaagaaagaaaagagatttatttgcctcatggttc |
| | | tgcaggctgtaagggaagcatagctccagcatctgcttctgggga |
| | | ggcctcaggaagctgttactcatggcagaaggtgaagcaggagct |
| | | tgcacatcatgtggcaaaagcaggagcaagagagagagaatgggg |
| | | cagggaagaagccccacacttttaaatgaccagatcgcatgagaa |
| | | atcactcgttacctcaaggacagtaccaagaggatggtactaaat |
| | | tcctgagaaatccacccccatgatctgatcacctcgtaccaggcc |
| | | ccgccttcagcattggggattatgtttcaacatgagatttggatg |
| | | gggacaacatccaaactatatcaccttgcatagtaggtagggttt |
| | | taaaaagcagtttggcacagtaagaaaagtacagattttttttgc |
| | | atcagacagacctgagttaaaatcccagcttcactgctaacatgc |
| | | taggtaaatgtgggcaagttaattaacatttctaagcctttgttt |
| | | cctcactggtaaaacaagtatttggaaatatcattgtgaagatta |
| | | gaaataatacatgaaaagatcctaggatgctgtctgtcatacagt |
| | | agtagtagtaagaagttattcttgccaaagattgttgagaatggc |
| | | agaattatctcagttctaagagctatagtttctaattatttgagc |
| | | ctagactcagattcatttggagcagctaactgctcaccaagagct |
| | | tattttccatcttaccaatgaggttatgtgccctgtgtttttaaa |
| | | atcagtctacttaaccaagagaacagaaatgacatgagaattaag |
| | | taatctcactttctctgttatttaggatttattcctactcaaaac |
| | | ctgagagttgctatgaattcaccattaaagcacttattaatatac |
| | | atgggttactgttataaatagcaatagtattgctattgtgtgagt |
| | | taggtgttgaagttcaagaaaggaataaagaatatttagaagatc |
| | | tttgaaaacagtgtctgggtacggtggctcatgcctgtaatctca |
| | | gcactttgggaggccgaggcaggcagatcacttgaggtcacgagt |
| | | tcaagaccagcctgggcaacttggcgagacctcgtctctacaaga |
| | | tatacaaaaattagccgggtatgttggcatgcacctgtaatccca |
| | | gctacttaggaggctgaagcacaagaatcacttgaacctgggaag |
| | | cagaggttgcagtgagccaagattgtaccactgcactccagcctg |
| | | ggcaatagagcaacactctgtctcgaaaaaaaaaaaaaaaaaaaa |
| | | aaaaagaaagaaagaaggaaggaaggaaagaaaaaaaaaggaaaa |
| | | aatgcaaggaaggtatttggtgaatctataataataaaaatgtat |
| | | ttgtcatttcctttttctgtgctctcattctataaaattgagtaa |
| | | aaaatctatatatagtttaaacacattaatagaaatcacaaaagt |
| | | tagctgagtcaacattgtagaaacataatatttctgtatgtcaaa |
| | | gaaatagacaacattaaaaagcagaaagcaattacaaagaatgat |
| | | tataagaaacatcacaaagggttaatattttaacacatttgaaac |
| | | tcaaaaatcactgagaaaagcagtagacttccataaatattttat |
| | | agagtagaaaaaataggccaagcacagtggctcatgcctgtaatt |
| | | ccagcactttgggaggccgaggagggtggatcacgaggtcaggag |
| | | ttcaagaccagcccggccaagatggtgaaaccccatctctactaa |
| | | aaatacaaaaactagccaggcgtggtggcaggtgcctgtaatccc |
| | | agctacttgggaggctgaggcagggaattgcttaaacccttaaac |
| | | ccgggaggtggaggttgcagtgagccaagttcgcaccactgcatt |
| | | ccagcctgggcgacagaacgagactctgtctcagaaaaagaaaag |
| | | aaaagaatagaaaaagaatccatgggcaggcacagtggctcatgc |
| | | ttataatcccagtactctaggaagccaaggtgagaggatcaattg |
| | | aggccaggagttcaaggccagcctgggcaacatagcaagactttg |
| | | tctctattaaaaattttaaaattagccaggcatggtgacgcacac |
| | | ctgtagtcccaattacttgggagcctgaggcaggagaactgcttg |
| | | aggctgcagtgagctatgattagaccactgcactccagcctgagc |
| | | tacacagtgagaccttgtgtcaaaaaagtaaaaaaataaaaatta |
| | | gccaggcatggtggcacatgcctgtagtcccagctactcaggagg |
| | | ctgaggcaagaggatgacttgagtctggaagatggagactgcagt |
| | | gagctgtggtcatgccactgcactccagcctgggtgacagagcaa |
| | | gaccctgtctcaaaaaaaaaaaaaagaaaagaaaagaaaaataaa |
| | | taaaatttattcaaatacaaaagtgatgtggtttgactctgtgtt |
| | | gccacccagatctcatctccaattgtaatccccgtgtattgacgg |
| | | aggttcctggtaggagatgattggatcatggggatggtttcccct |
| | | ctgctgttctcatgatagtgagtgagttctcatgaaatctggttg |
| | | tttggtaggtgtctgtcacttaccccttctttttctctctcctgc |
| | | tgccttgtgaagaaggtacttccttctcctttgccttccaccatg |
| | | attataagtttcctgaggccttcccagccatttggaactgtaagt |
| | | caattaaacctctttcctttataaattaccgagtctcaggcagtt |
| | | ttttatagaagtgtgaaaatggtctaatacagagacttggtacca |
| | | ggagtggggtactgctataaaaaataacctgaagatatggaagcg |
| | | actctggaactgggtaacaggcagcaattggaacagtttggaggg |
| | | ctcagaagaagacaggaagatgtgggaaagtttggaatttcctag |
| | | agacttgttgaatggctttgaccaatacactgatagtgatatgga |
| | | caatgaagtccaggctgagatggtctcaggtggagatgaggaact |
| | | tattgggaactggagtaaacgtcactcttacatgttttagcgaag |
| | | agactggcagcatttttcccctgccctagagatctgtggaacttt |
| | | gaacttgagagacatgatttagagtatctggcagaagatatttct |
| | | aagcaccaaagcattcgagaggtgacctggcttttcctgaaagca |
| | | tacagttatatgtgctcacaaagagatggtttgaaattggaactt |
| | | atgtttaaaggggaagcagagtgcaacaaaagtttagggagtttg |
| | | cagcctgaccatgtggtagaaaagaaaaacccattttctggggag |
| | | aaattcaagctggctggagaaatttgcataagtaacgaggagctg |
| | | aatgtgagttgccaagacaatggggtaaatgtctccagggcgttt |
| | | cagaaaatcttcagggcagaccctcacaacacaagcctggaggcc |
| | | tagaagggaaaaatggtgtgagccaggcccaggcccaggccccag |
| | | ctgttctgtgcagccttgggacatggcaccctgtgttccagccac |
| | | tccagctccagctgtggttaaaaggagccaaggtacagctggacc |
| | | attgcttcagagggtacaaatcccaagcattagcagcttccatgt |
| | | ggtgttgggtctttgggtgcacagaagacaaaagttgagctttgg |
| | | aagccgctgcctagatttcagaggatgtatggaaacacctcgatg |
| | | tccaggcagaagtctgctgcaggggcagagccttatggagaacct |
| | | ctgctagggcaatgcaggggggaaatgtggggttggagctcccac |
| | | acagagtccccactggggcactgcctcatggagctgtgagaaaag |
| | | acgcaccatcctccagactccagaatggtagatccaccaacagat |
| | | tgcactctgcgcttagaaaagctgcaggcactcaatgccagcctg |
| | | tgaaagcagctgcaggggctgtaccctgcagagccacagaggtgg |
| | | agctgtccaaggccatgggagcccaccccttgcattagcatggag |
| | | acaggggatcaaaggagattttggagatctaagatttaatgaatg |
| | | ccctgtcgagtttcagacttgaatggggcctgtgacccctttgtt |
| | | ttggccaatttctcctatttggaatgggaacatatacccaatgcc |
| | | tgtacccccattgtatcttggaagtaactaacttgcttttgattt |
| | | tacagactcaggcagaagggacttgccttgtctcagatgagactt |
| | | tggacttgaacttttgagttaatgttggaacgaattaagacattg |
| | | gggttctgttgggaaggcgtatttggttttgaaatgtgagaagga |
| | | catgagattttggaggggccaggggtagaatgatatggtttgact |
| | | ctgtgtctccacccaaatctcatctccaattgtaatccccatgtg |
| | | tcaagggagggacctgatgggaggtgactgaatcataggggcagt |
| | | ttcccccatgctgtttgcatgatagtgagggagttctcatgagat |
| | | ctggttttttggtaagtgtctgggcttcccccttttccctctctc |
| | | tcctactgccttgtgaagaaggtacttgcttctcctttgccttct |
| | | gccatgattgtaagtttcctgaggtctccccagccattcagaact |
| | | gtgagtcaattaaacctcttcctgcctattctcaggcagttcttt |
| | | atagcagtatgaaaatggactactacagaaagtgtgtaactttaa |
| | | actcagtagtatccaaagaagtaatgaaaatggagaaacgaacaa |
| | | caaaatcatagtacaatatggtgtatgtactaggacaggaagagc |
| | | ccttttaagaagagatctatgtatttccatttgtttatctctgaa |
| | | agaaagcaactttgccttgtattctgaaaaagaaaggaatatttt |
| | | attttacttgtaaaaatcttacaaggatgctagtctaaatatagt |
| | | tttcctaatttgccagagaatccatgaagatcgagttgataacaa |
| | | gatcagtgaagtaaaggtcagtgagttaatctcacagcagctgca |
| | | ggctaattccatttccagtgaaaaacgtcttgattgctcaccaca |
| | | tatcttttcaccacaaacagtttcagtcttaagatcacatgttgc |
| | | aatccatgagaagtaactattaagccttcaactatgactggaggg |
| | | ctcctcgccctttctgataaattgactggacaaaaactcaatttt |
| | | aaaatgacaagaaatagaagatgtataaatgtactttaaatgtga |
| | | ccaaaatgggttgtgaaaacacaagacacaatatccaaaaatgct |
| | | ggcaacacagtacactgtagagtattggttgtttatttacccttg |
| | | ctattgtgtggctgagcttactgccactgcccagcattgcaaggg |
| | | catcaaactgcctatcactagcctaggaaaagatcaaaattcaaa |
| | | attctaagtacagtttctactgaatgcttatcacttttgcaccat |
| | | tttaaagtaaaaaaatcagtaagttgaaccatcatatatccaaga |
| | | ttgtctgtatataaatattatacatctttctctcacttttaaaac |
| | | aaaataatactagccaatactaccattctcaaaagcacttgtgtc |
| | | aacagcctttaccccttaaagattttcctcacaattttaaaattg |
| | | ttacttactattttctttgaaatgttgaccaaacctggattaaaa |
| | | gatttgggggttttagtgactgtatttcacaaactctcttattga |
| | | ttctgcagcctcacttctgcctcctaaaaagccctcaccaaggtc |
| | | acgggggatggctcttttcagcctcttcctggcatttggtccagt |
| | | ggcatttggcattctaggacttccctctttgtctttgataactcc |
| | | ctctcttcctgtgttcctccttgctgtgttcacttgcttcgcttt |
| | | cttctttctgaagcatgtttacacagtgttttctctgattgggcc |
| | | tgtgacgttctttaggtcatcttttccacaaataatgcttcaact |
| | | agtacttgcgtgccagtgactccacgtcccactcatgagctctga |
| | | acctagtaccagcttctgctggacatctacaatgggatctctcac |
| | | aggcctctctcattggtaacatgccccagcctgaactcatctccc |
| | | acccatctatccagccatgctctctagttcacctgaacacttggg |
| | | tgtcatcctagatgctttcccttcccagtcttctgtgatcattct |
| | | gcctcatcagaggctctctaatctgtcttctttcctatatcgctc |
| | | ttgtccctattttaatcctaatcatctatttcctgacttattcat |
| | | tccttaagttggtcagtaatttaattaaaaacagatttaggccct |
| | | gaccttaaatgtgataagtgatatgaaaggagatgactggggaaa |
| | | aggatttccctcaaggaaggcctctgtgaagcctgaagcaagaat |
| | | gaaaacgagtcagacgaagagagaattgtatgaatgaaggctctg |
| | | aggcaggaaaacactcagatcattccagaatcacttagaagccaa |
| | | gtgaagccagttcctggagagcagatcatcaaatgaagatggaaa |
| | | ggtgaccaggggccagacctgtagttttggtgggccttggtgagg |
| | | gatttacagtaggacaccccatggtttaagtatgaaagtgacaag |
| | | attcctttaagttttaagaggcctcgaaatatgaaccacagatta |
| | | gatggaagctactctccctgtgtctggactttttagaatttccaa |
| | | gagctgctgtttctggaaccagattaatacaagtcagtcttccat |
| | | ttatttatttatgtatttatttgagacagggtctcactctgtcac |
| | | ccaggctggggtgcagtggcatgaacacagctcactgcagcttgg |
| | | gggctcaagagatcctcctgcctcagcctcccatgtagttgggac |
| | | cacaggcacctaccacccagctaattttatttgttgtagaaatga |
| | | ggtctcattttgctgcccaggctgttcttgaactcttgggctcaa |
| | | gcgatcctcctgcaacatcttcccaaagtgctgggatcactcttc |
| | | catttaacatgctatctcaacgtcaagataaactttaaaatcttt |
| | | agataataggctggcattttacttaaacgatctttacttcttcag |
| | | aactgccattccctataaatatctggttcttcaaccacatcaaac |
| | | cacttgtgatctcaaaaagcctcagcgtacactgtcctttctgtc |
| | | attctaattcctcctcatccttcaaaatcaactcaaggaccagat |
| | | ccagggagaagcttagtggtgcccacccgaaccggccccctcctt |
| | | cgagttgtgctgccattcgggcccacctcttcacacagggttgtc |
| | | agaccagaccagctcatgcgttcaccgcccttgcagggatgggat |
| | | gcagctgtgcacctctcagtgccgacacctggagagtctcacgaa |
| | | atgttgacaacatggcctgtttccatttcttgttcactaggactt |
| | | ctcatttactaacacacagaatttcctgtagtatgtccacttaat |
| | | cagttcaagcctaataattccttgatttgggtatagtgctttgca |
| | | tttatatactgatggtccccaacttacgatggttcgatttatgat |
| | | ttttcaacttcatggtgatgtgaaagtgatacacattctatagaa |
| | | accacactttcaattttgaattttggtctttttccaggctaccat |
| | | actctaaagatagagccacagatcccagtcagccatgtgattatg |
| | | agggtaagcgaccaatactctacagtgtattgtattgccagatgg |
| | | ttttgcccaactagcctaatgtaagtattctaaacatgtttaagg |
| | | taggccaggctaagctgtgttgctcattcagtaggttaggtatat |
| | | taaatgcattttcaacttatgatattttcaatttacaatgagttt |
| | | atcaggatgtaactctactataagtcaaggatcatcttgtatagc |
| | | acttttaatttatagagtcccttcaaatgtttgtttgtattttat |
| | | ttccactgcatccctgtgaggataccataagttatacagctaaca |
| | | aaacagttagttttcctgtgcaaagtgatggcttcatcttgtggc |
| | | agattacctggaatactgtggccaaggcatcttagttctactgtc |
| | | tttatatatctagtacagttatatttttatggcagctctgatttc |
| | | ttctttggcccaagggttattaagagagggaaaaaatttaatttc |
| | | ttaacagatatatatatctatgtcaagtcatatatttaattcaaa |
| | | cccttaatattcctaggtaatttttgtctactttctctgtcaaag |
| | | attgaaagatacagggttttaagtttccaactgtaattgtagttt |
| | | tggtaattgttttattactaacaggtattgctctgtatattttga |
| | | tgttctgttattcaatacataagaattcattacagttgaatcatc |
| | | atgtatagtataatttaccaatgtaaaataacctttttatcaaac |
| | | tgagtattcaccttataatgtcctctgtcagaggattataatacc |
| | | acttaaaaccttttaaaaaatattttgttcctgataattttagct |
| | | ttgagtggttttcttataacaattctagagatgcgtttttatttt |
| | | ttaaccaagatttaaagctttgtgatttagtaagagtctaaacca |
| | | ttcacagttcatgcttttttgtcaaatttctattatttagtattt |
| | | tcctctcttttatattttcctgttattttccatttctattctttt |
| | | gttagactggaaattttttctttgctttcttttatcctagtgatt |
| | | tgaaatttatgtaatatatactattctacaataccctttatttgt |
| | | tttcaatatttgaacctatatttttcaacattattaagaataaaa |
| | | tagtatttgttgctattttgaaatgatagaccatgtttttaaggc |
| | | aggttggtggttgttaaggcaccagtatcggccaggcacgatggc |
| | | tcacacctgtaatctcagcactttcggaggccgaggtgggcagat |
| | | cgtttgagcccagcactttggccgatactgtggtttactgtattt |
| | | tgttctagtttattttatggaaaatgggaattcagtggttaagac |
| | | aaggattaaatagcagaagaaagatgtgtacatatgtacagatgt |
| | | atgtgtcctttatatgttttttagtactcttgtctcctttctggt |
| | | cctcatttaaggttatctatttcatgcagtaaatttttcttcaca |
| | | attcatttcatttagagagtgaatgctaccttccaagtgggcttt |
| | | ctccagttttcctttcagggacttaaaggagaagtgatgttaaca |
| | | gttttatatttccattgcattttacagtgtgcagatgtcttcaca |
| | | tatatttccccatttgagctttacaaaagcccttagtattattct |
| | | cattgtctagattccaaaatcaggcttagaggagttaaatagttg |
| | | tccaggatctcaagatgcaagacccacaatcatgaacagaggcag |
| | | atgttcaggatggaggcaagctgaaactcaaaaccaaatcattat |
| | | gactccaaattcaggagtcttttagctgccacctgcatgggctct |
| | | tggtgtagctgaccaccagagtttgtagagctgtcattcaggtgt |
| | | gccatggactttcctgggacctggcacaggagaaggactgagtta |
| | | atgtttgctgattaaatatctgttacaggctgggcgcggtggctc |
| | | acgaccgtaatcccagcactttgggaggccgagcagggaggatca |
| | | cttgagctcacaagtttgagaccagcctgggcagcatggcgaaac |
| | | cccgtctctacaaaaaatttgaaaattagctggccatggtgatgc |
| | | atgcctgtagtcccaggtactcaggaagctgaggtgggaggatca |
| | | catgagcccatgagattgaagctgcagtgagctgagatggtgcca |
| | | ctgcactccagccttggccatagagccagaccttatctcaaaaaa |
| | | aaaaaaaaaaagttacaataatcttcccttcaaagctggaaggca |
| | | ttatttacctgtctgtccagcagatggtgctacataaccaaggga |
| | | atctgttgcttgcccttggtgaagctattaaagccaatacagatc |
| | | ttgagaatttcaaaagcaaaaatcaatactggattatgagtgctc |
| | | taggaaaataaagagataaattttcaatttacatacttatatata |
| | | gttataccatatttgtaaataaaaatatataaataatttatcaaa |
| | | attcctttttaacagcaacaaccacagtaaacccacaggttaaaa |
| | | actccacaacagtctatattaatcagtcaatgcaaagtacattcc |
| | | aattccaagttaactgaaaataatcaacttaatcatttggttggc |
| | | tctgagcagccttcactgcttgctcttgtgtcatgtttctttctg |
| | | tcctcgattggctatagacttacagacggcttttgcagaggacag |
| | | tgtactcatgtccatcctttgcatcctttgtgggagttggctagg |
| | | cagcactctccctggggacactatgaactcctcttcttgagtgca |
| | | gagatcacgtcttgttcatcttcatgcccaataccttgttccata |
| | | aatatgaatggattagaattctaaactcttaactctgccccaaga |
| | | cagttctgagaggtagtaagtcatataacacctgaagaggactgt |
| | | tcttgtcctaattacattaggttataagatgacaggtgagggagc |
| | | caaaccagggggcctggaaattattcatacatctctagatacagt |
| | | atacaagttgtgtgtattatgtgtatttactctgtaattgattgc |
| | | ttgagatgaacccccaaacacactcgtgtttggatcattattatc |
| | | tacccttctccttaaataatcttaatttcctatgatgcttgaaag |
| | | ggaaagaggggccaggtgtggtggttcacacctgtaatcccagca |
| | | ctttgggaggctgaggtgggagcatcacctgaggtctggagttca |
| | | agaccaacctgaccaacatggtgaaaccctgtctctactaaaaat |
| | | aaaaaatcagctgggcatggtagcacatgcctgtaatcccagcta |
| | | cttgggaggctgaagtgggagaatcgcttgaacctgggaggggga |
| | | ggttgcagtgagccgagatcactccattgcactccagactgggca |
| | | acaacagtgaaactccgtctcaaaaaaaaaaaaaaaaaaaaggca |
| | | gagtggggaagagagctgcatgaaggagagatttactaaatagta |
| | | cttaatcccaaaataatttctataggtttgaatatgatccctgaa |
| | | atttattataggttcaggtaagtattaatcacgggtattcagaac |
| | | tgtggtttaaaaaatgtatagaacatgtttccttccccttgaaac |
| | | tttttatcagctaattataggaattatattatacctgcaatcatt |
| | | aaagtccagaatgagacagtacttggtaaagtgctgaaatttata |
| | | ataaatgcattatagcaatccagttaaggaggaagagccaccatt |
| | | attgaacatttgttaagtgtcagccattgtactagataaatttta |
| | | gttattatttttatttaggcaccaaaaaatccatgggatagttgg |
| | | ttatccccatcttactgaagaggaaactgaagctcagaaagttta |
| | | agcaacttgcacaggtcacatagcaagtaaggagcatggccagga |
| | | atcagaccctgatctcctttggtctactaagcttgcaaaggatct |
| | | tcccgcctccttccaagaccattcaatattatcagtaaatgtcca |
| | | tggcaaggatgtagttcgagttatagggttccattcaagatatga |
| | | ttggtaggtgggaagcagatatgtctgtgtcaatcagtatcctgg |
| | | aagaaggagatgatgaactcaagtggtgattaagggaagtttaat |
| | | gaagggactatttacagagatgtggtggggttaagagaaccaaca |
| | | aggggaagtgatgcactcaaaaagttactacctccaggctttagg |
| | | ggattgggggagggagtggcacagtgtgaacccagtgtggttgtg |
| | | agaaaagggattccctcagcagccatggccaaggttagagtctcc |
| | | actgccaaactgcatccaggtggtgagggaatggagaataggggg |
| | | gggtaacaaactctgacctcggtatccccaaagggcaaaggattc |
| | | caggtggtacagttcgtaaagattagcctcagggcacagaacagg |
| | | gcagagaagaatggagaattgatctggaggaaacaaacaatggct |
| | | tgcccatgttattgcagggagagtaggctggtgtgcacagcagga |
| | | gggtggggagcccagcatatagctgtgttggggcctgtgcagatc |
| | | agcctcactggcagggaggatctgagccgagaggtggtggaagat |
| | | gaaatcgagtaggcatgttggtagtcctaaatatcaagtaaacgt |
| | | tcctgatcttacattgatactcaatagtaagccaattttgtttcc |
| | | cataagccaatattaatattacgtatttcttttataagccagaga |
| | | tatagagagataccctagaagaatgataggggaaaggaaggcaag |
| | | ggtgagagaagaccttgtgtgaatttgtccaaaatgtttatccac |
| | | agGAACAATCCCTTTGTGAAGGCTGCTGGTATGTGAATGTGTGCC |
| | | GGTTCCCTTGGGGCGTTCATTTGGATCTTTCTGTGTTCCAGTGAC |
| | | CTACGGCATGATGAGCTCCAATGTGTACTACTACACCCGGATGAT |
| | | GTCACAGCTCTTCCTAGACACCCCCGTGTCCAAAACGGAGAAAAC |
| | | TAACTTTAAAACTCTGTCTTCCATGGAAGACTTCTGGAAGgtatt |
| | | tgcaaataactttgaaagtacctctctatcacagaaaattgttca |
| | | tttggcttcatcatttcaatgcatgagtatcgacaggacctgctt |
| | | tgcatttaacactgtgtgagacgtaagttatggtgagttgttaga |
| | | agttactgttcctactctcaaagggggtaaactaacattgagaac |
| | | tttgcctgtgccttgcactgtgctgagtgtttcatatcttacctt |
| | | atttaatttctatagtctaactctataaggtaagtactaagacta |
| | | tgccctagtttgttaatgaggaaaatgagattcaggatgtttaaa |
| | | tgcgtatggtcacatggctagggaacaagaaaaattgattttttt |
| | | ctagcctgacagctacttcatcctagtttgtaattcattccatga |
| | | gtcaagattcaataaatatttattgagaatctcctagaatgtaag |
| | | gccaatgaagggcagtgtggttcttctgtcttgcttcgccttttg |
| | | tgttttgtctctttgttgatgatggcatgtatccccagctcttag |
| | | aacagtgcttgattcaaagtaagcacattctttcaaaggtctgct |
| | | gttggtggggcttggtggctcacgcctgtaatcccagcactttgg |
| | | gaggccaaggcaggaggattgctttagcccaggattttgaaacca |
| | | gctgggcacaacatagtatgactttgtctctccaaaaaagttaaa |
| | | gaattagcagggtgtggtggtacacacctgcagtcccagctactc |
| | | aggaggctgaggtgggagaatcacttgagcgattgcttgaggtca |
| | | aggctgcagtgagccatggccatgctactgcattccagctggggc |
| | | aacagagtgagaccctttctcaaaaaaaatcccccccaaaaaaaa |
| | | acccaaaaacaaacaaaaaaggtctgctgttgtgaagttcaaccc |
| | | aatccagccccttcccaagttgtcacaaattccaacgtagttaac |
| | | agtataccaatgagtgataccacaggaaaaatattaaactgatct |
| | | gagggatatggggcttggaatctaagaaaattggaagggaaattg |
| | | aaaaggaaattattatttctccttggggagatagtttctaaaatt |
| | | cttactacaccctggggtcagagctgttgattttaaggatagaga |
| | | caactgagtcacaggaaactattcatatataaaagtacctggcat |
| | | ccaaaaccacacttgtataatatgaatctttcaccatctgagtag |
| | | ggcaaatcagtctatctctgttgatcatctgacaaggatagcaca |
| | | ctgagaaatagatctgtcttccctacaggcatagctagttgtaca |
| | | aactaacaagagacttttgtatacacattccatgatgataaatgc |
| | | caatcactaaagggacgaggagggattggagagttcaccatacag |
| | | caaaatagtccagacaggtgaaaggtctatcaaatgccaggctgg |
| | | taatcaaaactgtagccttttctctaaacaaagtttagaaccatg |
| | | attgtgtgggacattattttaataagggaaagtgcagttaatcat |
| | | gaccccacctttagtccaagaacaaaaatcagagctgccacgtat |
| | | taagtacccactctgtgccaggtgcagtaactatgcaaaagatgg |
| | | gttttccagatgcaagaaccttggttcagaggaccctgctcaagg |
| | | cctcatagctaacaaatgatggggcaagatgctatcccaaatctc |
| | | tctgacaacaaaactcattcttatcactctactattttcatagag |
| | | ttgccaaatgcttggttatgcaaacgatgcaggcaggggcaagac |
| | | agcggctgagcttggaactttttcagagatgtttcctttgctttt |
| | | agTTCACAGAAGGCTCCTTATTGGATGGGCTGTACTGGAAGATGC |
| | | AGCCCAGCAACCAGACTGAAGCTGACAACCGAAGTTTCATCTTCT |
| | | ATGAGAACCTGCTGTTAGGGGTTCCACGAATAC |
| | | (SEQ ID NO: 240) |
| | premrna | AATGGTAGTAGCCACTGGGGAATCATTGCAACTTATAGTGGAGC |
| | ENST0000 | TGGCTATTATCTGGATTTGTCAAGAACAAGAGAGGAAACAGCTG |
| | 0508588.5 | CACAAGTTGCTAGCCTCAAGAAAAATGTCTGGCTGGACCGAGGA |
| | | ACCAGGGCAACTTTTATTGACTTCTCAGTGTACAACGCCAACATT |
| | | AACCTGTTCTGTGTGGTCAGgtgtgtactgaggacatgcatccct |
| | | cctatttctgtgtggttgtacatacatcctattctggggttagcc |
| | | agaaaaacctttgcctgcagttagctacatgaggatgccaaggac |
| | | ccagacggatagcaagggaggggtaaaaactgaaggcttaccgaa |
| | | ataaaggatatttgaggaagggagttgggatcctagaatattacg |
| | | agttggaaagaaccataactctggtccaagttcatctcaatgctg |
| | | gaacctttccagaaaaagtattgtgtttttctaacatctgtcttt |
| | | acccattataaggatggttagtgccacatgttccatcaccaagtc |
| | | ccccggccatcaaatcttgactcatttcctggagtttctcactct |
| | | cagatgagcctctgctattagcacacaagcacagtaaccggagtg |
| | | cttgtaggatgctcagtaggatacccaggttacctgctcgtgctc |
| | | agggctaccaaaggcacgtaaagttccttccacagatcctgggat |
| | | gttgccatgatgacccctctgtgagatagtaacaaaaatgacaaa |
| | | gattccactggcttgtctgggactcttcttcattcatttattcag |
| | | caaacattcattggacacttaatatgggctaggcattgttctcgg |
| | | ctcttgggacatgtcagcaaacaaaataaagatccgcaccttggc |
| | | agagcttgcatccaaccaggaggagactagagaataaacattaaa |
| | | caatacaaataaatagtatagtatattagaaggtaataggtacta |
| | | ttaaaaagaaagaaaaagcagagcaagaggaagtcagagttcacc |
| | | cactttaatcttcctggtgagcatgtcagcaacacccaaacatca |
| | | ctaacatggattattgcatgtatatttacacataagataagaagt |
| | | gtttattctcataatagtctttgtcatcattcttgaggttaagtt |
| | | caattctgctttatgtggcttgttggattgtcccagtccttgtat |
| | | ttaacaacatttgcagaaaatagtaccacattaaatcaattatag |
| | | attatcccttatccaaaatgcttaagaccagaatgtttgagattt |
| | | aagaaatttttcaggttttggaatgtctgcatatatataatgaga |
| | | tatcttggggatgggacccaagtctaaacacaaaattcatttatg |
| | | tttcataaatgaaacttaatgcacatagcctgaaggaaattttat |
| | | ttttcccttagggatgttgaatcaactgttgtgtgccagcatgtt |
| | | gacaatgacctgtcacatgaagtcgggtgtggaattttccacttg |
| | | tgcgttcatggcggtgctcagaaagtttgggattttgaagcattt |
| | | tatatttcagattttcacattagaaatactcaagctgtccttgct |
| | | cacagtggccaaaaaaaaagaaagaaagaaaagaaatactcaacc |
| | | agtagtccagtagtagttatcactagaaatgaatgaaaatctatt |
| | | gcagtattattgagtttttcctaattattccagtgcagataaaaa |
| | | gaaaagaataaaaaggaagagaataaaaacagagaggcaactctg |
| | | atattttagtaaattctatttatagaaggtcttgagtatttcttc |
| | | tgcttcctcccttactttaaggatgaacattgttaagacaactgc |
| | | ttcatttctctatactgtttttctaagtttctggaagtggttgac |
| | | tactgcagggccagaatgggccagagaaatgacttgacacttgaa |
| | | ggccacttccttcccttttgagttcccaatgaagctgtcacatac |
| | | aaggctcttggcttcagagttgctctcctgagtttttgattctca |
| | | cccctactctctaacacatcaaataggaaagaaagaacaggagaa |
| | | ctgacaatgaaaaggaaggaaaattttcacacttctctgaccagt |
| | | tctaatttaccatagtcctgtttttacttgattattgctcatgca |
| | | tgtgtcctgtatgctcaggttccaggtgcggctacctgtcctgta |
| | | atggcagagatagtgatggctagtagctgactaaagggcttttaa |
| | | atgtctcaaaatgaagcagctagagattctatttctagttagaaa |
| | | agaagtctgtatcattaactgaatcacccagctttctcagtgtga |
| | | cacctacaaaatgggcatttgacaagaaaaaaaccctcagtccag |
| | | ttatggtaaagcagtaaagatcagagccatcagtatggatgtaaa |
| | | atagtgtatgttttagacaatcagacatctattgagtacccacct |
| | | attaactataaggctctgggaagaagagagaaaactaccctggag |
| | | gacaaactatttgatgctatttaggtgttacataatgaatgaatg |
| | | actcagttcctatctttatatatgtacaaaatatatcctacttct |
| | | caaccagattacacatgttttgagtggatggtttatatttcttta |
| | | tattcttcatgttgcctagtagaaggacttgaatttaatagaagt |
| | | cctagggccaggcatggtggctccttcctgtaattccagcactct |
| | | gaaaggcccaggcaggaggatcatttgagcccaggagtttgagac |
| | | caacctgggcaaaagggcaagactcagtctctgccaaaaaaaaaa |
| | | aaattagttgggcatggtgctgcacacttacattcccagctactc |
| | | aggaggctaaggcaggagaatcccttgagccctggaatttgaggc |
| | | agcagtgagctatgattgcaacactgcactccagcctgggcaaca |
| | | aagcgagtccctgtctcttaaaaaaataataacagaagtcctaga |
| | | aaagtttgtgtgttgatttacttttacattaaaagtatatggcat |
| | | gttgagcagcgtaaatatagaaaagtgtagggaagactgagcagg |
| | | aagtactcctttgggactgaaagacctcaggaagtcttattcctt |
| | | tgatggcacaaaattctccaagtatggaattattagctatgataa |
| | | aaatgttttgccgctagtttggggggactcatggtagcagtttca |
| | | ttaccttgtaatgcatgaacagaacagatggacatccattcctgg |
| | | ctgtattcatgtgttgttgttgttattgttttaattgttcttatt |
| | | tacatgcaggttattggttgaattcccagcaacaggtggtgtgat |
| | | tccatcttggcaatttcagcctttaaagctgatccgatatgtcac |
| | | aacttttgatttcttcctggcagcctgtgagattatcttttgttt |
| | | ctttatcttttactatgtggtggaagagatattggaaattcgcat |
| | | tcacaaactacactatttcaggagtttctggaattgtctggatgt |
| | | tgtgatcgttgtggtaggtttgagaacaacaccaaatttcctatt |
| | | ctattctacaagcatgttaactagagtctttgatctcctcagcat |
| | | tgtggatcttgatattcccaaaaaagaatctaaaagtccccctca |
| | | attatatcaacttctgttactaattattttctcattttgcatgag |
| | | taactttgctgagtatgaagtggagaggtatttacagtatgctct |
| | | cagccacgctaataacaagagtatctcagtaattcatatttggct |
| | | ttagtatgccgtatgagatgtggaggagaaaacagttttttttct |
| | | ttgtttttttttccactaatgatatttttcttcaactgctggtaa |
| | | aaatcaatttatattttcctgcacatgtgtgaagttacagcaata |
| | | aaaaaacttgtcggccaggtgtggtagctcatgcctgtaatccca |
| | | gcacttggggaggccaaggcaggaggatcacttaagccccagagt |
| | | tcaagcccagccagggtaacatagtgagaccctgtctttacaaaa |
| | | aaaaaaaaatttaattagtcaggcatggtggcacacacctgtagt |
| | | ctcagctattccagaggccgaagtgggaggatcatttgagcccag |
| | | gaggctgaggctgcagtgagctataaatgcaccactgcactgcag |
| | | cctgggtgacagagtgagaccttgcctcaaaaagaaaagaaaaaa |
| | | gaaaaatcatcctgaaaatattttgtgcggcagagaaaactttct |
| | | gcagtttaaaattttctgcaaatagtctgcagagtacaaatgtaa |
| | | gttatattcatcaaagttttctgtatgaggtataagaaatcaaag |
| | | gcaggccatgcacagtggctcatacctataatccctgcactttag |
| | | ggaactgaggtgggaggatcacttgagatcaggagtttgatacca |
| | | gcctgggcaacatagtgagaccccatctctaaaaaataaaaataa |
| | | aaaataaatcaaaggcagagtcataatcaagaccatgacaccatg |
| | | taaattctgtgtctgctcttgactctattataacttctaagattt |
| | | ttttcaagatgttttcccttcatccttatcacttaattaagcatc |
| | | cgtcacttccttcctgtggtttcagtgtataaaagaatttttaca |
| | | agcttttctcccttcagcaataacaggtaacatttcgctaagtcc |
| | | agttgtacatttaagcatataacaacatgcttaattattagatgc |
| | | ttacaagctttgcttggcataggtgtaccatgtattattctatgt |
| | | ctttccttcccactgtcctatgatagccattaccttctgaaatct |
| | | cagtaaatgatgcactaccctattagcattctctcttctgttagc |
| | | cctccttatgagagttattctttccctcatcccactcctaaaaat |
| | | tcatttggcctttgtggagtatttagatcaagtcattattaaact |
| | | attccccactagaattattaatagttgataaaatatggaaaatat |
| | | attattcatatgtgagtgaaagaacacttaaagcataaaaaagaa |
| | | ctacatggccatatattgcatggcaataactatattagcagcaat |
| | | aataattgtaatagtaataataataatggctaacactttagaagc |
| | | ttctgtgttaggcatttccgggtgcttttcatggtacagaagctc |
| | | atttgttcctcatactaaccctcttcactactctgccactgcctc |
| | | tcaagccatggatatgtgcactttagagtttttacttaaagtaaa |
| | | aattcctgtagaatagatggtgttgaccccatcagaccgtgatgc |
| | | agttagaagtgcatcccgtcttttacgatggccatacaacatcac |
| | | acaaatcaaggtaaacccttgatccccaaattctcaatgtattaa |
| | | taatgaacagagttacacaagaattttagcatttaaggaaacagg |
| | | agagacgataatactggaaataatttttcagaatatttctgttcg |
| | | gattgatggcagagtgcaggccatatacattgacaattattccag |
| | | aacacaattattgtttggagctaaaaggatgcaaaccctgcctct |
| | | tggcttatatggatttatttatgtttaggccattgaaataatggt |
| | | agaaaggtaagtatgatatgctaattaagaacagacttccttttt |
| | | atattttaaccaaagaactcaatatcaacaaaagactagtcagtg |
| | | gtattcaccctttttgatcatacacacctatcagtgaaagctttg |
| | | actactacacaccctaatttattatctttatttgtaaattgtgtg |
| | | cctactttggtgtaacaggtacatccgtaaaacatacacatacat |
| | | agaattttaaagattggaataaaagttaatacaaatagaattcca |
| | | aaactttcttaccacctacctagaagttgtaacattttcttccca |
| | | tagcccagtgaattgccttgcacctgctttggcgaccaataggaa |
| | | gaaaggcaatgatatcatggaaattgtctctttggctcaacagcc |
| | | acagcattttgcaagtgtttctttgaaaacttgcttcactgtgtt |
| | | tttcaattttttttttaaacaacacttaatactgccagacaacaa |
| | | ttcagacagtgtgctttttgtttataaagaacaaggaagggctac |
| | | tggaatctcacttgtcctttgaaacttttgctgaccaagtgtaga |
| | | agtgagggcatgccttctgctcacccacaaaacacaccctgtgct |
| | | ccactggaccttcaaaccagtggaaagacccaacgctttttgttt |
| | | tatctagccaaaatttgcttatgcttacccaaaacctgaaaagaa |
| | | attatattcttaatataaatacaatcacatcttgaaatcactttg |
| | | aaattttctttattttccttttttctttccccaacatatgttctg |
| | | aagtacacaggctgcatcagtcagccatttgtcctgagcaatagt |
| | | ctttcaaaactagaagaattacttgaaaaaaagaagactattaag |
| | | gaatttaaactcaaataatttattgaccacttgctaggttgtaca |
| | | gggtaaatttttttttttttttttttttttgagatggagtcttgc |
| | | tctgtcacccagactggagtacagtggcaccatctcggctcactg |
| | | caagctctgcctcccgggttcacgccattctccctcctcagcctc |
| | | ctgagtagctgggactacaggtgcctgccaccatgcccagctaat |
| | | tttttgtatttttagtagagacagggtttcaccgagttagccagg |
| | | atggtctcgatctcctgacctcgtgatccgcccacctcagcctcc |
| | | caaagtgctgggattacaggcgtgaaccaccgcgcctggcccgac |
| | | taattcatttattcagcgagtgttttctgagcacctactatgtac |
| | | ttgccactgttctaggcactggggatacaggaatgaacaaaatca |
| | | cccaaatctctgccctcccaattttctggtatggagagaaagttc |
| | | ttttgtagatagggatgagaatcccacagaaaactcaggagtgat |
| | | gtaacaatgcaaatgctcacaaaatctctttcctcatctttcatt |
| | | ccctatttggaaggaaaggtttcaaagacattgatgttgtataaa |
| | | tgaccatcttcttcattattttataaacatttgtcctgtgcagag |
| | | taaaaacaactggactgcataacaaattatacctattgagagttg |
| | | ggtaaagagttaccattggatccagtccaccaacccaaatgtttt |
| | | tctacatgtattagctgagatgagctcctcatctcaggagaagcc |
| | | tattcccactgcaggccttcttgagtctgctgtgttcatcattcc |
| | | caccaccaacacaaaaatacaagattggctctggaaatccttcac |
| | | agagaaggaaggaaaggaagatggtgaggttggctttttagctgt |
| | | gatcagcaaccaagctggtctttgctatgagaatcagtgggacca |
| | | tgatctctatggtcatctcaggaagggagggctaatgaagtggtc |
| | | tctggccatgattcctaagaagagagaatggacagcaaagatcgc |
| | | agcacctgccacagcccacctgctgcagattcagaccccctggtg |
| | | gggccaggttgttatgactaacccctagcactgtctacatttagt |
| | | ggtgatggatgccaaggagggggcagtgtccccttggatctgatt |
| | | gtaaagcttagaaccaaagcatatgtggaaagttgtagggtcatg |
| | | agttaagggacagaaatgagcaagagagaagcccttggctcatat |
| | | attcccactgcaggccttcttgagccctctgtattcatcattccc |
| | | actgtctttcaagcccaagctcatgctttgttctcatgggcactc |
| | | atttttaggatatcttttgtccctcttgactttatgttgtgtgat |
| | | accaactctttagtagttttttgtattatttaacttcatgtgttt |
| | | atctgctcttgagtctcgagggcaggatttatgcagtttacttct |
| | | gtttattcctcttagcccttgcacagtgctgtatacatagacatc |
| | | tactatatttttgttaaacagagcaaagaatggctggcaattgga |
| | | gaatgcagagaaaccgaaaaattttaaatttaaaaatcacaaata |
| | | aaaacagcaggatgaaggcaagaaagcaaaagggtggaaagtgat |
| | | taaaatgaaggtggcagaaaaaacagaaagcattcctctttgagt |
| | | ttgagtctgttatagtgtgatctcttctgtgtatgcatgtatgtg |
| | | tgtgtaatgtatatgtgcacacacatgcatgcatgccttcgttga |
| | | gtttctattccgaactaaggaaatgcaagcaatatactgttttac |
| | | ttattttatggcagggcttaacactttccatttgagtgagtgact |
| | | tttaagaatgacatcgggtaagtataatggtgagcccttataatt |
| | | aatacattggtgaagaaaaatatactagtcatattaaggtaagtt |
| | | tcatatttctaaaacactgtaataaaatataaatattttgctttt |
| | | cagCTGTCAGTGGTAGCTATAGGAATTAACATATACAGAACATCA |
| | | AATGTGGAGGTGCTACTACAGTTTCTGGAAGATCAAAATACTTTC |
| | | CCCAACTTTGAGCATCTGGCATATTGGCAGATACAGTTCAACAAT |
| | | ATAGCTGCTGTCACAGTATTTTTTGTCTGGATTAAGgtaatttat |
| | | aaatttcatgttctacattttaaataatattttctttaaaaaaaa |
| | | tgagttccacaaaatcatggaatacttgaatttgaaattcaagtg |
| | | accagccaaagctgctcaatatttactttgagacagggtctcact |
| | | ctgtcacccaggctggagtgcagtggtatgattacagctcattgc |
| | | agcctcgacttcccaggcccaagcgatcctcccaccttatcctcc |
| | | gaagtcactgggactacaggcatgtgccaccatacccggctaatt |
| | | tttaaattttttcgtagagacaaggtctcattatgttgcccaggc |
| | | tggttttgaactcctgggttcaagcaatcctcccacctcagcctc |
| | | ccaaagtgctgggattacaggcatgagccaccgtgccaggcctca |
| | | tattttacatataaagtaaactattgagactcatgtgatcattcc |
| | | tctcactgtcaatgacatacttctgctatctgaattagtgcaaga |
| | | tcagtccctataggttttgtttaacaaatgcagtaagaggccttt |
| | | cagtgtgttagctgggcctggggccccaggctgctaacagatgag |
| | | atgaacaggtgaaggaaaaggaacttagagaaagagagggaagga |
| | | gcaggtggagggaaggggagagttgctgcacttggaaatgcttgc |
| | | tagaagggatcgcctcttttccaggtagaggctgtaagggaagct |
| | | ttacctagaattaaggttggaacagacactgcttccaaatagttc |
| | | cttgctcactattttccttattgtcccaagatataatgtgcattt |
| | | ccatgtgtgtgaaaggttatgacatttcatatacaacaagcctca |
| | | attctggagatgcaggaaatttcaataattctcaggcagcagctg |
| | | ccattcggtcaccagcacaggctctgattgtgctgtccagacagt |
| | | aagtactagccacatgtgcctatttaaattcaaatttaaattagt |
| | | taagcttaaatacaattaaaaacgcagttccttggtcctactggc |
| | | cacacattaagtgttcaatggctactgtctaggacagtggaaatg |
| | | tagaacatttccatcatcacagaacgttctcttgaaaagcactgt |
| | | tctggaaggtacttacccgttatgtacttttctgagttggtattc |
| | | atacctagaagacctgaggtttatcacaagacatagacttggacc |
| | | aggcgcagtggctcatgcctgtaattccagcattttgggaggccg |
| | | agataggtcccctgagcccaggagtctgataccagcctgggcaac |
| | | atggcaaaacctcatctctactaaaaatacaaaaattagctgggg |
| | | gtggtggcacgtgcctgtagtcctagctacttaggaggcttaggc |
| | | gggaggattgcttgaatccagaaggcggagggtgcagtgagccaa |
| | | gatcgcaccgctgcactccagcctgggcaacagagtgagaccctg |
| | | tctcaaaaaaaaaaaaaaaatgcatagactttatcctgtatttct |
| | | catgctatttatttattgacatgcttgttcaagagaaaccatcac |
| | | taaagcacaaaaccttgatcataacatagtaataataatcaaaca |
| | | gcaaaaataataatagtaataagaatgttctgtggtgatggaaat |
| | | gttctatattttcattgtcctagacagtagccactaaccatgtat |
| | | aggcatggaacacttaacatgtggctagtaggaccaagggactga |
| | | atttttaattgtatttaatcttacttaatttaaatttgaatttag |
| | | atatccacacatgtttggatagcacagtcagagcctgtgctggtg |
| | | aaaggatggcaggtgctgcctgagaattactgaagtttccttgat |
| | | tattattagtttaataataataatcaagatagtaataataatcaa |
| | | gatagtaataataatcaagatctcagctgggcacagtggctcacg |
| | | cactttgggaggctgaggcgggcagatcacctgacgtcgggtttg |
| | | agaccagcctggccaacatggtgaaaccctgtccctactaaaaat |
| | | acaaaaaaaaaaaattagctgggtgtggtggcacgtgcctatgat |
| | | cccagctacttgggaatctgaggcaggagaattgcttgaacccag |
| | | gaagcagaggttgcggtgagctgaatcatgccactgcactccagc |
| | | ctgggcaacagagcagcacttcgtctcaaaaaaaaaaaaaaaaga |
| | | tctcaaatgaattgggattgtattaagtaatgattaagtaatgtg |
| | | attacagcaatcctcaagaaatatttcactgtggccagtaacaat |
| | | gtgtaacagacctttaaacttctagagattttcctacaacatgtg |
| | | tctcaggctgatgtgttttatttagtgcttctcttggaaatgtct |
| | | tgcccctcgatactttatcattaaggtctttaaggcagggatcat |
| | | gactctacttttttttttttttttttttttgggacggagtcttgc |
| | | tctgtcgcccaggctggagtgcagtggcacaatcttagctcactg |
| | | caacctccgtctcctgggttcacgccattctcctgcctcagcctc |
| | | ccgagtagctgggactacaggcggctgccaccacgcccggctaat |
| | | tttttatatttttagtagagacggggtttcaccgtgttagccagg |
| | | atggtctcgatctcctgacctcgtgatccacccacctcggcctcc |
| | | caaagtgctgggattacaggcttgagccaccacgcccggcctcat |
| | | gactctacttctaatatctcatcatgtgctcttccactgaggctt |
| | | ctacttagagctacacaatctgggcagccatcctcagtgccttat |
| | | ctaccaacatgctcaatatggctttgcagggttcactgtctacca |
| | | gcagggttcactatctaccaacatgctcaatatttctttgcagtc |
| | | aggcagagcaggctttgcagttcaggcagggcagctggctgcagg |
| | | ccccagctgactcctggggatagaatgccaatatttcagacattg |
| | | cagagatttgaggcaatgtacataaagccctccacatataactga |
| | | tgcacaataaatgacagttaatattatgcaacaagaatttcctgg |
| | | ggggttttataattaatttttatttgtgtgaagttttttccctcc |
| | | cttttactttaatcctttttggggggaaagcatcactagtcacag |
| | | ttcacggcagcctcgacctcccaggctcaagcaaccctcccacct |
| | | cagcctcctgagtagctgaaaccacaggtgtgtgccaccacacct |
| | | gactaatttatttttattttctaatgaaacagaatcttgccatat |
| | | tgcccaggctgatcttaaactcatgggctcaagcgatcctcctgc |
| | | ctcagtcttccaaagtgccgggattatagatgtgagccactgcac |
| | | tcagcctttttttttttttttttaattgtagatagcataaaacct |
| | | actgttttaaccatgcttaagtgtacaattcagtggcattaagta |
| | | cattcacagtgttgtgcagccatcgccattatgctgcattatttt |
| | | cagaactttttcattattctaaactgaaactttgtatccattgaa |
| | | cactaactcccaattcccccagtccctggtaacctccattctact |
| | | ttctgtcactgtgagtttgactattctaagtacctaatttaagtg |
| | | gaatcatacagtatttgtccttttgtgtcaggcttatttcacttt |
| | | gcatgatgttttcaaggttcatccatgttgtaacctgtcagaatt |
| | | taatttcttttcaggatgaaataatgttttattatatacagtcac |
| | | accattttgtttatccattcatctattgatgtcttctctctctta |
| | | cagCTCTTCAAATTCATCAATTTTAACAGGACCATGAGCCAGCTC |
| | | TCGACAACCATGTCTCGATGTGCCAAAGACCTGTTTGGCTTTGCT |
| | | ATTATGTTCTTCATTATTTTCCTAGCGTATGCTCAGTTGGCATAC |
| | | CTTGTCTTTGGCACTCAGGTCGATGACTTCAGTACTTTCCAAGAG |
| | | TGTATgtaagtatatatgaaattaagaagaaaaatttaatcagag |
| | | ttgtcactgcttctcaagaataaatcttcatatgaggttgctata |
| | | tgaccaccaattatttaaaaccagttattttaagtaagaattaat |
| | | taccttttcccaaaacattgatctacccatgcaaagaagacaatg |
| | | catcctgaaatgctgatgcttaagatagcagcccaaagtagtaaa |
| | | atacagttaacagacataggaaaccaacactgttctgaagactga |
| | | gtttttctttgcaccaaatgcagatggtagcttctagaaggctgt |
| | | ttgcctatattcttactcctgttgaatattgttgccatatattta |
| | | gaacttcaagttattttctaaggaaaaaaacaagatatttctaat |
| | | attctaaggtaaactcagaccagtacaagaattttcagttttttt |
| | | ttccaaagatcccaaatgtgaaataaaacaacaaaaagcagccag |
| | | tgtcagatttctatgccatttagaaaggagttagtttaaaaagga |
| | | atggaagtaatagggttttgtgcatagatatctcgaattaatatt |
| | | gctgttgataaaagtgattttgctaagacccagcactgacaacac |
| | | ttggccactttgatcccattttaagtacttgtcagaatattggat |
| | | ctttgaactcaaaccattttgggtttttggggtttttttgttttg |
| | | tttttttttgttttgttttgtttttgaggcacggtcttgctctgt |
| | | tgcccaggctggagtgcagtggtgcaatcatagctcactgcagcc |
| | | ttgaactcctaggctcaagcaatcctgctgcctcagcctgctgag |
| | | tagctgggactacaagtgtatgccaccatgcctggccaattttta |
| | | actttttttatgagaagggatctcactgtgtagcccagggtggta |
| | | ttgaactccagggcctcacactgtcctctcacctcagcttccaaa |
| | | agtactgggattacaggcatgagccaccacaccaggccctgttgt |
| | | tttttttttaaagaaatttttaactttagaccgagggtgactgtt |
| | | gtcaaggtttagggttaagatgttttacctagattatgtgttgaa |
| | | atgttatagccaattgctttataagttattgaataataattgtat |
| | | tttctttttttttttttttttgagatggagtctcgttccatcgcc |
| | | caagctagagtgcagcggtgtaatctcagctcactgcaacctctg |
| | | cctcccgggttcaagcgattctcctgcctcagcctcccgaatagc |
| | | tgagattatgggcgcacgccaccaagcccagctaatttttgtatt |
| | | tttagtagagacggggtttcactatattggccaggctgttctcga |
| | | actcctgacctcgtgatccgcccgcctcggcctcccaaagtgctg |
| | | ggattacagacgtgatccaccgtgcccagcttgtgttttcttttt |
| | | aaccaaatggaaataacctctgtagcatgaaagcattttattatt |
| | | attgcagaaggctttaattgctgatacaagtagcaagactttgta |
| | | aatgggattgacaattttctgttattcggcagctacctatactgc |
| | | taaaaggtccaaaaataatgaaatcatctttaagaaatgttgcat |
| | | caactagtggacattctttgtttttgtattgtggtgttttgtttt |
| | | atttttatagCTTCACTCAATTCCGTATCATTTTGGGCGATATCA |
| | | ACTTTGCAGAGATTGAGGAAGCTAATCGAGTTTTGGGACCAATTT |
| | | ATTTCACTACATTTGTGTTCTTTATGTTCTTCATTCTTTTGgtat |
| | | gtacatttttatttatagtgaggttcaatttaaacttcgtaaatc |
| | | cttgtcttctcttttctctcacactttatgtcctatcaattttaa |
| | | ataaagacccaggaagtagaaaaaagtgtggatgttggaaaactt |
| | | attttccttttattaattcacagttttgagactcatatcaaatgt |
| | | cttttctgtggtctttcattgatccatgtatatgtgtctattcaa |
| | | tgcaaaaaaaattagatctcttccatggtctttcatttctctctc |
| | | tatatatgtatctattccatgcaaaaaagaaattagatcaagtac |
| | | aaatttataaagatacctaaaatagtgctttgcctaaaaagtaga |
| | | atatgcttacatgctttttaaactcatatgtcagcactttcgtag |
| | | tcacttgctagcatgacttttctctctttcttcttttctttttaa |
| | | aaaataagaacggaaaagcaagctagatctaagatgtcgagtaat |
| | | agttgagtgaatcattgcatgtcaaaattaggatattctgtttta |
| | | aattatttatatcccattcatctagagactgcctacagagaatat |
| | | tcaaataattaagtttaaaactaaatgtaacaatgaatggaaatt |
| | | gcattaaaattattttcaaaaataatttttttattctcttgattt |
| | | ggtacaaatgaacatttttaatgtttttgccctaagtcaattaag |
| | | tttttttaaggtgttttgttctttttcttaacatttatatattca |
| | | attgtctactgagaaggtgttaagccagcttaatttaggcaatat |
| | | ttttcatctaaacactaacagtcatcttaagaacaattttcttaa |
| | | gaaaataacattttttccatttcagtaaattgtgtaaagatccct |
| | | tgaggaaggttaagtgatcacattttcagtaattcagtgtaataa |
| | | ctctaaagtcagtccaggtattactggttaagtatatggtattta |
| | | ttgattgggtattagatgtactgtattaatttcctgtttaaaaaa |
| | | aattttttttccggggagacacagcctctggtgtaaaacaaaggt |
| | | gtgttccctagctgtactttaacaggactgaaaaggtcaggaata |
| | | tcattcaagttcatatgtatcttgctgtatgcatggtttatggct |
| | | catttttaaacttacacctcttaagcttcttcttcctatcatata |
| | | ttaaaacaatggagagaagaataagcctctgttactctaccattg |
| | | atagtacttcggattctagagtacctgaatctctactaagaaggc |
| | | aaaaaccaggaattgagagtcctgcacctgacccttcagttgatc |
| | | tcaggccacctagttttctccgtttatcaatctgccaaacaagga |
| | | tggatagagtcgtggcaactggaaaggctcaaatgtggaattgtt |
| | | tgaatgtggtcctttagtaggcagccatcttaccagatctagagt |
| | | attcagtcatcttaccagatcagtcaccagaacatgaaaagaagc |
| | | tcttagtttctatctttatactaaaattgtttttttgtacgactg |
| | | cacaaaaaagaattgctctccttgcacctcccagagatataggtg |
| | | gatagatacatacatacgtacatacatacatacatacatacatac |
| | | atacatacatagacacatacatagatagaagtctactttcaatac |
| | | aaacctgtcttttaaggaaatgacaagctgagcatagggttggcc |
| | | acctttctgagccgattgcctggtattagtttattgcccctgttt |
| | | agcaagaaggcacagtgttaagaagtggctcagctgaaccaggat |
| | | aaccccactcttcccccacatcaacaggaaagacatcctggtgca |
| | | gatgtccatctgataattcagggaacctcgggagacaggatggag |
| | | aggagggtgagctagcttcctcttcccacaccttcaagagccttt |
| | | ctcaagcactttctattttttgaaatctctttagaggtcccagac |
| | | tttgatctgtttcaattaaggtattggcaggcattagttaacagc |
| | | cacttggaagcaaaaatagaacattagatccctgagttggaagag |
| | | agaaggtagaaggtgttacttggactcaattatctgcacttggaa |
| | | ttgaggcatttagtcaaaaacttatatgtattctatattctattc |
| | | tcatttctgctacagaattgtaaacaatattcttccttaatacag |
| | | aaattcatagcccactaaaataagagcgttctcatttgttcattt |
| | | ctcaatcatttaataagtatttactaagccactatatccatatat |
| | | atatatatatcatatatactgtataatacacactgtagtgttttt |
| | | tgtggattgtgtactatgaggtagtatgttagatactgccagtac |
| | | tggggtaaggaaaacagcctgattaggcccttacgaagattcctc |
| | | agacttgtggggaaaacagacattatcaaatagaaatacttgcaa |
| | | accacagttatgtgttaaaaaggaaaaacaaagtaaaaaaaaagt |
| | | tggtgggggggaacctgatctcctggatacagtgcttcgagaaag |
| | | tttgttgttggaaatgcaaaccattactactgtggaagggaaagg |
| | | tcagaaaaatgaactcaccattactgaatagtaatagtagctatc |
| | | aattaggtggcacttacctgcatcaggacctgtcctgagcacttt |
| | | acatagattgtctcactaaccagcccaacaaatatgtaagggaga |
| | | tactattatttttcccattttattaatgtaaaacaattaaataat |
| | | tctttaaaattagacttagaaaagtggagcaacaatcttagcagt |
| | | gctaggactgaaatccaagtttgcttgactccaaagtctatctct |
| | | cttccagaaactttttctttactatctgcctagtaggcctgctgt |
| | | attcctatttgcaacagccttttaaactctttaaaaatgtgtcct |
| | | gtaaatttcatatatgattatacaaaaaaacttggaataagcata |
| | | caattctacttatctgtgttaactgttgaaatttgaagagctttt |
| | | tggaattctatacccttcagtagtgtatgtaaaagtttctaaata |
| | | tagagaacatagataagcaaaaataatattaaataaaataatcgc |
| | | accattagtaggtaaatatactaatattttgttgtattttattct |
| | | tgtatgttttcacaaagtatatcataaaatttttcctgtggcatg |
| | | acttaacggagaaaataatcttcccaaaacatgtggcagcaaaac |
| | | tgttaatttattacatcaggctgggcacagtggctcacgcttgca |
| | | atcccagcactttgggaagccgaggcgggcagatcacttgaggcc |
| | | aggagttcgagaccagcctggccaacgtggtgaaacactgtctct |
| | | actaaaaatacaacagttagccaggtgtagtggcacatgcctgta |
| | | atcccagctactcaggaggctgagactcaagaattgcttgaaccc |
| | | aggaggcagaggttgcagtaagctgaggtcgcgcccctgcactcc |
| | | agcctgggcaacacagtgagactctgtctcaaaaaaaaaaatttt |
| | | tttttaaataaataaataataaatttatgtcttcataaagcactc |
| | | agattaggaaaaaaaggataaacaaaaaggcatgtgtcatttttt |
| | | tgattgataattccaaattatgtttcttcctttaatttttgccct |
| | | cctttcatttacaaacagAATATGTTTTTGGCTATCATCAATGAT |
| | | ACTTACTCTGAAGTGAAATCTGACTTGGCACAGCAGAAAGCTGAA |
| | | ATGGAACTCTCAGATCTTATCAGAAAGgtaggaaaaaccttaatt |
| | | ctcagaattcttctgtttctgacataaaatgagcattgtttcacc |
| | | cagattttcaaatcaacattgatccattgaaattgtttgaaataa |
| | | agaatacattgctatatttcaggaataatttaaatgttccctatc |
| | | ttggagtcttgatggatatactgctatcttgaattttaattctgg |
| | | gaatccttttatgccctggaattaaattctcaacaatcttttgac |
| | | actttaagagctgagctgaaggttcatcaccttcattattttgac |
| | | atctcctgtagctggctctcacttcaggatcctgagttgagaata |
| | | aactagaagggaagattatataaagggatttccacctcttctgtc |
| | | tcaattaccattttaaaaaaataaaaagttttagaggaaaacact |
| | | tagtagttcaccctttacccttgaccttccacggcagttttaaaa |
| | | taagcaaaggaaaagattcatgaattcaggccatagcctggggcc |
| | | tgagaacttttacttatgcaccttctcaggaagggtttcattgtt |
| | | aaatagaagggcaggacaggaaagttgggcctctttgttcttctc |
| | | aatgtaacttctttatttggtttaaagtataaaatgtatacaaca |
| | | acaaataaccacatttaaaatacacagtttgtttcccaacatcat |
| | | tttgctaagtcatagtggctccttaactgtaatttttttttttat |
| | | tagtccaagccttaggattatgttatctgtgatatatgttataat |
| | | agaaaacttaagcctcttaaaacaaagtccttgggatgggaccta |
| | | agattcacattatcttgattccgcataacagttgcttacatttta |
| | | gcaaatctccagtgtgtatgcaagcactcctcacttggcacaatt |
| | | ctgatacacacaaactttggttaccccagttttgttatgtaacac |
| | | caccttcaacaacacagttcaaatttcagttatcatagtatatta |
| | | actctgagtaacagcacaaagtacaaactccactgctagctcttc |
| | | agtgtatagatcagttacctgagtaacagatgtgcaggctgagca |
| | | ggctcactggtcagtcatgacactattttcagtctgttactaatt |
| | | ggtcactgagcatctgctatccaattcacaaacaaagaaagcatg |
| | | tagtgttgcttccttgtgttccagtaataagcccatgtgacattt |
| | | tacaaaaatggataattgaaaaagagaatgggtgcagtggctcac |
| | | gcatgttgggaggccaaggcggatggatcacctgaggtcaggagt |
| | | tcgaaaccagcctggccaacatggtgaaaccccatctctactaaa |
| | | aatacaaaattagctgagtgtggcgacagatgcctgtaatcccag |
| | | ctactggggaggctgaggcaggagaatcgcttgcacctaggaggc |
| | | ggaggttgcagtgagccgagatcgtgccattgcactccagcctgg |
| | | gtaacaagagcgaaactccgtctcaaaaaaaaaaaagaaaagaaa |
| | | agaaaagaaaaagagaatgggctagcaaagaaatgaaaaatggta |
| | | acactggaagtgaaaatcaaaacagagtaatggatttatagaaga |
| | | aatagctgagtgaagaagaaataggagtgttgacactgcgatcat |
| | | tcaagagatccagatatggagccagaagaacttagggcaggtcta |
| | | tcaacttaaatgaggaaaatagctgtgataaaacagatgaagatg |
| | | tcttgaggaaatgatgcctgcaaaaaacttcacattaagggaagt |
| | | cttattagagatatttcacaataatgaaagtacaaaagaaaaaat |
| | | gttggggctggacatggtggcttactcctgtaatcccagcacttt |
| | | gagaggccaaggtgggtggatcacttgaggccaggaagtcgagac |
| | | aagcctgatcaacatgatgaaaccccgtctctattaaaaatacaa |
| | | aaattagccagacatgatggtgcacacctgtaattccagctactc |
| | | aagtggctgaggcacgagaattgcttgaaccagggaggcggaggt |
| | | tgcagtaagctgagattgcaccactgcaatccagccaggtgacag |
| | | ggtaagactgtgtctcaaaaataaataaaagaaaaatatgttgga |
| | | agctcatccacatttaagaaggaatatgacaattcactaatgcat |
| | | agaaaagaagttcactccacattgtaaagtgtacagtgtaatatt |
| | | atacaatgaaaacaaggcaagtgctgtttaaactactctggatac |
| | | attttttacaaagaaataaaacactttagtttttaatgtttctaa |
| | | tgttttacattttagtgtattaaatcaatattagttttcttcttt |
| | | tttaagctccctatacatttataactgacactaagggagtgttta |
| | | atgttttgattaaaagttgtaaagatcacagaacaattgtaattc |
| | | ttcccactgattattcagatcattttgcacaatttcagcttgcat |
| | | ggtcacttacagtgccgcactatgtgcaaagcaaggtcaggtcta |
| | | aagttcgctaatgaaaaatcctcggccaggggcagtggctcaccc |
| | | ctgtaatcctagcactttggaaggcgaggcaggcagatcgcttga |
| | | gctcaggagttcaacaccagcctgggcaacatggtgagaccctgt |
| | | ctctacaaaaaaaaaaaaatagcaaggcgtggtgactcacacctg |
| | | tagtcccagctacttgttgggggctgagttgggaaaatcacttga |
| | | gctcaggaggtcgaggctgcagtgagccagaatcacgccactgcc |
| | | cttctgcctgggtgacagagtaagatcctgtctcaaaaaaaggga |
| | | aaatcctcatctacatttcactgggttttttgtttgtttgtttgt |
| | | ttgtttatacacacttaaggaaattactgtctagaagatagataa |
| | | tataaaaaataaaaatgcaattcatgattcgggtttcttggtatt |
| | | cctaagaactgttgcacagtactttatgctctgaggcagacagct |
| | | atagcatatatagtaatttttgtttctatcacataaacttgaata |
| | | cacatatgagtaaaagacctttagttcttcatgacttactgaaag |
| | | accctgactttttccatgtaactgttccacaagtgttttatggaa |
| | | aactggatacattaattcttcattcatccagcacgtacttgttga |
| | | atggccaatgtacaccaggtttgtagtagttactactgtgaatgg |
| | | aaagtaaaacagatgcaaaaggagaatacactaaaccaagtcttt |
| | | tattttttctctctctgatagGGCTACCATAAAGCTTTGGTCAAA |
| | | CTAAAACTGAAAAAAAATACCGTGGATGACATTTCAGAGAGTCTG |
| | | CGGCAAGGAGGAGGCAAGTTAAACTTTGACGAACTTCGACAAGAT |
| | | CTCAAAGGgtgagaatcatgcttcctgaggttctgaaaaattcct |
| | | gcttctaaagataaattcctggtgataagagtatttctagcccaa |
| | | gggctcatacagatactttttttttttttttccagaggcaggtat |
| | | ctttctggaacatgttataagaggaaaacttgcccccatttggtg |
| | | atttctcctttcctcctgcattttgatgtctctgtgttgagggtg |
| | | aactgggtacaaggaatgatttttatctgtatcctctctctaatt |
| | | tcagGAAGGGCCATACTGATGCAGAGATTGAGGCAATATTCACAA |
| | | AGTACGACCAAGATGGAGACCAAGAACTGACCGAACATGAACATC |
| | | AGCAGATGAGAGACGACTTGGAGAAAGAGAGGgtgggtctggttt |
| | | aggagaaccggatttgatttggtacctacaacaccacagatgtat |
| | | caaacactatagaagtagtgggttattgagtctcttgcccattcc |
| | | ccaccacactctctctctctctcagtcggtttatgtgttagtacc |
| | | ctgtttattccagaaagaatatataacacaattatgtataaaaat |
| | | gggtggttagcatgatataaaaacgtcaaaatgaaaagcaagcaa |
| | | aacaaaagtaaaaataatggattattaatgaagcttaaaaatgca |
| | | ttcataaaaacacatatgcttattaagattgggctacaaattggg |
| | | ccctaagcttgctggtaatcagcttgaaaagagaagcctgattag |
| | | ctgcagagtccacaatgtccgtgagagtgaagaaaacaaaaaatg |
| | | acttaccaagagatgtgaaattattctggttagttagtggctatt |
| | | taaattgttaacttttttttcttttttttttttttttgagatgga |
| | | gtcttgctctgcctcccaggctggagtgcagtggcacaatcgcga |
| | | ctcactgcaacctccacctcccgggttcaagcgattctcttgcct |
| | | cagcctcccaagtagctaggactacaggcacatgccatcatgccc |
| | | ggctaatttttgtatttgtagtagatatggggtttcaccatgttg |
| | | gtctcaaactcctgactgcaagcaatctgcccaccttggcctccc |
| | | aaagtggtgggattacaggcagtagccaccgtgcctttcctaaat |
| | | tattaacatttataataaaattaacagccgccttccatttgaata |
| | | ctttttacaaaatagttaaaaataaacataagtgggcttttatag |
| | | tcagaaaaaaaaattcaaagctttaccattaactttcaaaaataa |
| | | atggttagacagcaacaacaaaaatctgtggtaactgaggtacag |
| | | agaacacagatgaatgttattacaaaagccactttcctatgagaa |
| | | gtctaggacagtggtttctaaatgccactccacagacagtgctag |
| | | taggtgacagacttctccagtcacagtgaaatttaagcataaaga |
| | | aaatgaggaaaatttttacaaggctctatttagacaaagttctta |
| | | ttctgacattacatctttcctactttggagctgttgaatgtatta |
| | | tcttttatgaaaagaaggcgatccaggttgagcatccctaaccca |
| | | aatatgtgagtctgaaatgctccaaaacctaaaacttcttgagca |
| | | caaacatgatagtcaaaggtcatgcttaaaggaaatgctgtcatt |
| | | ggagcagtttggattttgggttttcagattagggatgctgaacca |
| | | gtaagtataatgcaaacattccaaaatatttttgaaaatcccaaa |
| | | tccaaaacacttctgatcccaagtatttcaaataagggatactca |
| | | acctgtaatatatttcttcatttctttatttattttattattatt |
| | | ttaagatggctcatggcccactgcagcctcaaactcctaggctca |
| | | agtgatcttccgacctcaacctcccaggtagctcaggtagctggg |
| | | actgcaggcatgcatcaccatgcctggctaattttttaaaaaatt |
| | | ttttgtggaggcagagtctcaccttgctgcccaggccagtctcaa |
| | | actcctggcttcaagcagtactcctgcctcagcctcccaaagtat |
| | | taggattacaggtgtgaccactatgcctggcccatatttcttcat |
| | | ttagttttttctttgcctgctgtgtttttaatgttctttcttgtt |
| | | caaacaaaaagttggctattccttgctgttagttaaatttgccaa |
| | | tctatgaaactgaaaaatgcaggagtcccagcctggtgttaaata |
| | | caaagaaatcccaggtaaatggcatgcacccagttcctgcttgcc |
| | | caagtccttggtgaggcttctgtggggtctcagtgttctgctcct |
| | | cactcagtgaccccttgttcttcagGAGGACCTGGATTTGGATCA |
| | | CAGTTCTTTACCACGTCCCATGAGCAGCCGAAGTTTCCCTCGAAG |
| | | CCTGGATGACTCTGAGGAGGATGACGATGAAGATAGCGGACATAG |
| | | CTCCAGAAGGAGGGGAAGCATTTCTAGTGGCGTTTCTTACGAAGA |
| | | GTTTCAAGTgtaagtataaaggaattggcagaatttgcgttgaca |
| | | agagtccacatgagaccaggcagttccctcatctctctgaattca |
| | | ctcctttccattactaatcatccagcttttaaaaataacttatac |
| | | tggccagacgcagtggctcatgcctgtaatcccaccactttggga |
| | | ggccaaagcaggcaggtcatgaggtcagaagttcgagacgagcct |
| | | ggccaacatagtgaaaccccatctctattaaaaatacaaaaaatt |
| | | agctgggcatggtggtgggcacctgtaatcctagctacttgggag |
| | | gctgaggcaagagaattgcttgaacccgggaggcggaggttacag |
| | | agagccgagatggctccactgcacaccagcctgggcgacagtgca |
| | | agactctgtctcaaaaaaaaaaaaacttgtcaattggtgttttgt |
| | | ttcttacataatatgtttactataaaaattagcaaataagagcaa |
| | | aagaaaacattaacatttcacatatttctaccaatgaaaaatgtt |
| | | tattaatatatcagtgtttgtgtctctatttgcatgtgtttatca |
| | | atgtttctatatatttttatggcctaacatatggttcgtcctgaa |
| | | gaatgttccttgtgcatttgagaagaatgaatattctgctgttca |
| | | agtgttctgtagatgtttgttaggtctagtttgtttacagtttta |
| | | ttcaggtctcccatttcctggttgatcttagatgtgcctagatgt |
| | | ggtattcacgtttgaaagtggggtattaaagtctccaagtattat |
| | | tagttggagttcatcccttcaattctgtaaggttttgctttgtgt |
| | | attttggaactctgttgttgggtgcatacatatttataactagta |
| | | tattttcctaatatattgaccctattttctctctcaacttaatga |
| | | ggctaaagaaaaaaaagaattgaccctgttttcattacaagatgt |
| | | tatccactttatctctagtaaaattctttgttttaagtatttttt |
| | | gtttgatattactgtaaccactccagctttcttttggttgctgtt |
| | | tgcatgataaatctttttccatccttttactttaaacttatttat |
| | | atctttcagtctgaagtatgtctctcctgtagacagcatataatt |
| | | ggatcttatctttttatccagtttgacaatttctgtttttgatta |
| | | gattgcttaatccattcatttaatgttatcattgatgtagttgat |
| | | ttctgtctgctattttattttttgttttctagcttactttttttt |
| | | gttcctctttcactgctttcttgtacattaagtgaatattttcaa |
| | | gtataacatttaaatttttttaatgatttttcattttttttagtc |
| | | aggagttgctctaagacttagtttatacaattaagttatgaaaaa |
| | | ttacttcagatatatattaactgaatccagtgagatatagaaatc |
| | | atttctatttagcttttttcctcttccctctttttgtgctatata |
| | | ttcatctatctatatgtatatatagtcatctacatatgttgcaaa |
| | | tcacattgttagaacaatgttacatttttataacacactgtgtaa |
| | | tatatagtatataattttatatctcttaatgaagctgagagaaga |
| | | ggaagtatatatttataaatgtgtatattaacctacttttttacc |
| | | atttctaattctcttcttttgctcctctggattcaagttatcatc |
| | | tgtcgtcattctctttctccgatacagctttgctactgcctacct |
| | | cctattattgtcaaatatattacatttctattatagaccttcaga |
| | | tccaattatgtacatatttttacacaactgctttttaaatcagtt |
| | | aaggaacaaaaggagaaatgtacatatatactatattttatacct |
| | | acacagttatctttaccagtgttctttgccttttcatgtggattc |
| | | tgattactatctggagtcacttgctttcagcataaagaatttcct |
| | | ttagtattttttgtaaagcaggtttgctagcaatgaattctttca |
| | | ttttttgtttatctgagaatgtttttctttctccttcatttcctc |
| | | tggcttgtattgtttctgatgagaagacaggtgctaattttactg |
| | | tggtccccttgtacatgatgactcaattttctctcaccactttca |
| | | agatttttttgcttttgtctttcattatttttactgtgatatgtc |
| | | tgggtataaatctctgagttcatcctacttagaaagtgttttttc |
| | | tgcttctttcactttctcttctcctttgggacccgcattatgcat |
| | | atgcttaggggtatcacatatttctcttaggctctgttcgtcgtc |
| | | attttttttccctctctgttcctcagagtgcatagtctgtattga |
| | | tgtatcttcaagttcactgacttgttcttctgtcagcttgcttaa |
| | | atctctgttgagctcctctagttatttatttatttatttatttat |
| | | tttatatatatatatatatatatatatatagagagagagagagag |
| | | agagagagagaaagagagagagagagagacagggagacagggtct |
| | | cactccatcacccaggctggagtacagtggtgtaatcatggctct |
| | | ctacagcctgaacacctgggctcaaatgatcctcctgcctcaacc |
| | | tcccaagtagctaggactatgggcacatgctgccatgcctggcta |
| | | atttttaaaaaaaaatttgtagagatggcatcttgttatgttgcc |
| | | caggttggtctcaaactcccggcctcaagtgatccttccgcctcg |
| | | gcctcccaaagtgctggggtcacaggtatgagccaccgcacttag |
| | | cctgaattttttatttattatacttttcaactccagaatttctat |
| | | ttggttctttttagcaatctctgtctctttattctgtatttgatg |
| | | atatcctgtatttgatgagacaatgtcatcataacttcctttttt |
| | | ttttaagagatagggtctctctctgtcacccaggctagagtgcag |
| | | tggcatgatcctagctcactgcagcctcgaactcctgaactcaag |
| | | caatcctcccacctcagcctcctaagtacctgacactacaggcat |
| | | gagccactgtacccagctaatttttattttttgtagagatggggt |
| | | ctaagttgcccaggctggtctcaaactcctgggctcaagtgatcc |
| | | tccctgctcaggctcccaaagtgctgggattacaggcatgaacca |
| | | ctggacccagcctcctttatttctttaaccatggtggggtttttt |
| | | attatttgtttgtttgttttaacctgtttgaatatatttataata |
| | | ggtaccttcaggtctttgtctgctaagtctgacatctgggccctc |
| | | tcaaagacagtttcagttgtctttttttttttcttcttgtgtatg |
| | | ggaacattttcctgtttttttgttttgttttttgttgttttgttt |
| | | tgttgttgttgttgttgttaaagattggacatgttagataatata |
| | | ttgtaggaactctggctagtgattccctttcctccccaggacttg |
| | | tttttatttctctttgcttgtttactttttcatacagtctgtttc |
| | | cccacagtgtctgcctctgactttattccttagagggcgcagctg |
| | | tggccatgtacgtagtcactctgggaagacagtggttttagcagc |
| | | gtgctcattaactttctctgacctctttgttatacctcctgcctc |
| | | tgtggatattagacccagttattacatttcattgtttactgattg |
| | | gtctattgttttccagaatgccttgggatgtaatttgctccacag |
| | | tctgatccacttaaattcaggcctctttgcagggctagttttaga |
| | | ggccagtctttttttttttttttttttttttttgagacagagtct |
| | | cactctgttgcccaggccggaatgtagtggtgcagtctcagctca |
| | | ctgcagcctcaatctctcaggctcaagcaatcttcccacctcagc |
| | | ttctcaagtagctaggactacaggtgcacgccaccatgcctggtt |
| | | aatttttgcattttttatagagatggggttttaccatgttgcccc |
| | | ggctggtctcaaactcctgagttcaagcaatccacccacctcagc |
| | | ctcacaaaaggccgggattacagatatgcaccaccacgcctggcc |
| | | cctgaggccagtctttaactgtcttcttagctgtctctttccctg |
| | | gttctctctggtgaactagctggtaatttgtttatctcataaggc |
| | | taccagattccttgtaaatgcttatccccacaatctccattgttt |
| | | tcaagagcatcgttagtctttaatttcctcacgcttcattccaaa |
| | | taaagtccattcacttgagaagagttctatattcctatgacctgt |
| | | gtctccccatgagcaaaactgctactgctttacagagccagggac |
| | | agtggcccacctctctgtggcatcctgctttatgaacaagtcact |
| | | gggctcagatggcagtctctgattttctcaccttgcttcttctgg |
| | | catggaaactccaccctacaagtgggaactgagtggaagaagaga |
| | | gccccattccccttagccactcttaacaggattagaacctctgca |
| | | acatgcatttaagaatgggaacaggctgggcacagtggctcacgc |
| | | ctgtaatcctagcactttgggaggtcaaggcaggaggattgcttg |
| | | agccaggagttcaggaccagcctgtgcaacatggtgagaccctca |
| | | tctctacaagaaatagaaaaattaactggtgggttgtgtatacct |
| | | gtaatcccagctactcgggagcctgaagtgggaggattgcttgaa |
| | | cctggaggcagaggttgcagtgagtcaagattacaccattgcacg |
| | | ccagcctgggcaacagagtgagactctgtctcaaaaaaaaaaaaa |
| | | aaaaaagagtaggaacattgaggctgggcatggtggctcatgcct |
| | | gtaatcctagcactatgggaggccaaggcaggagtatcacttgac |
| | | gctaggagttcaagaccaacctgggtaacatagcgagactttgtc |
| | | tctattaaaaaaaaaaaaaaaaaaggtgtaaaaaacttttgtaac |
| | | aagagtgggaaagccgggcacagtggctcacacctgtaatcccag |
| | | cactttggaaggccaaggcaggcaggcggatcacctgaggtcagg |
| | | agtttgagaccagcctggccaacgtggtgaaaccccatctctact |
| | | aaaaaatagaaaaattatctgggcatggtggtgcacacctgtagt |
| | | cccagctactcgggaggctgaggcaggagaatcacttgaacctac |
| | | gaggcagaagttgcagtaagccaagatcacgccactgcactccag |
| | | cctgggcgacagagcaagactctgtctcaaaaaaaaaaaaagagt |
| | | ggaaatgttaggatgagaaatgctggcagcctgcccctccctggg |
| | | agatactgtagccctagactggaagttgggggaggagggagccct |
| | | gtgctcttagctgcacccatgtgaagttgtgcttctatcacatga |
| | | gctggggacaggagagaaggctcagattatggcttcagtgccaca |
| | | gaatctcttcatactaaaatttagtagattttcttgaataaatgc |
| | | tttttcatttgctgtacacccttaaaagtttctagaaatttttaa |
| | | tatttgagttttaaaaaataattttcaacagttacagttatttca |
| | | ctaaagagagagtctacagaaccctcttgccaccattgcagaggt |
| | | tgtctttggcttacgagtttttaaagtatttgtatacatttttta |
| | | agttcaaaataatagaattgtaagtgaacatgctgttttcatact |
| | | gtttttcaagctttatttaatatattgtaaatctaattctatttt |
| | | attaaatagtctgccacagtataatgtctgatgtctccttagaat |
| | | tttattgtatggatgaacaatgattatttaatttcctaccaattg |
| | | ttgggtgttttttgtttgtttgtttgtttttgagactgggtctca |
| | | ctctgtcacccaggctggagtgcaggagtgcggtggaatgatcac |
| | | ggctcactgcagcctcaacatcccaaggctcaggtgatccttcca |
| | | cctcagcctcgcaagtagctgggagtacaggcacatgccaccatg |
| | | cccacctattttttagagatgaagttttgccatcctgcccaggct |
| | | ggctcgaactcctggcctcaagcgatctgcacacttccgcctccc |
| | | aaaatgccaggattacaggcgtgagccatcatgccctaccccccc |
| | | atcaattgtttgatgtagccatttttcaatgatccgcgattaaga |
| | | agcagcactcttttatagccaaaaattacacatatataaaatttt |
| | | cctttagaaaatgttctaaaaatggaatgtctaactaaagggtta |
| | | ggcatacattcttaagacttctgatacgcgctgacttgcaggaaa |
| | | gttgtttcagttaacactcctaccagcggcatccgagagttaatc |
| | | tgtaaagcttgagacaacttagaaagtgtttcaaatgattgtgtt |
| | | gcttaagaaaaaaatcttagcacttccttttgaaaagccagtggg |
| | | gctgaaaagacaatgacaagcactttgtccctctgtactgtgttt |
| | | tccttgcagCCTGGTGAGACGAGTGGACCGGATGGAGCATTCCAT |
| | | CGGCAGCATAGTGTCCAAGATTGACGCCGTGATCGTGAAGCTAGA |
| | | GATTATGGAGCGAGCCAAACTGAAGAGGAGGGAGGTGCTGGGAAG |
| | | GCTGTTGGATGGGGTGGCCGAGgtcagtagtcatgagctgaaaac |
| | | accgctgctgagcatggtgttattaatgaaaatatatgttgctga |
| | | cagttgtatttgaagtattgaagaagagtaaaaaaaatttacgtt |
| | | tatagaaattcacaatgatgtttccatttactctcattttcagat |
| | | ttttttctctgaaacagaaacactctttctataaaatctcttgct |
| | | ataaaacatcaatgtagtcatattgtctaacccttaggctgagat |
| | | gtttatctttctccataactacagataaaattataatctggaggt |
| | | gttactttcttaatactccatatgctaatggtcctgccttcactg |
| | | cagggtagaattaagtgaaaaattactccagcaactctgagattt |
| | | gctattatatgctgtaaatctccagccttaccaaactacagatta |
| | | tttggtccctggacttcctaaggcatttccttctactgcccccaa |
| | | caccagtttctttttccctttttagGATGAAAGGCTGGGTCGTGA |
| | | CAGTGAAATCCATAGGGAACAGATGGAACGGCTAGTACGTGAAGA |
| | | GTTGGAACGCTGGGAATCCGATGATGCAGCTTCCCAGATCAGTCA |
| | | TGGTTTAGGCACGCCAGTGGGACTAAATGGTCAACCTCGCCCCAG |
| | | AAGCTCCCGCCCATCTTCCTCCCAATCTACAGAAGGCATGGAAGG |
| | | TGCAGGTGGAAATGGGAGTTCTAATGTCCACGTATGATATGTGTG |
| | | TTTCAGTATGTGTGTTTCTAATAAGTGAGGAAGTGGCTGTCCTGA |
| | | ATTGCTGTAACAAGCACACTATTTATATGCCCTGACCACCATAGG |
| | | ATGCTAGTCTTTGTGACCGATTGCTAATCTTCTGCACTTTAATTT |
| | | ATTTTATATAAACTTTACCCA (SEQ ID NO: 241) |
| | premrna | ATACTTTCCCCAACTTTGAGCATCTGGCATATTGGCAGATACAGT |
| | ENST0000 | TCAACAATATAGCTGCTGTCACAGTATTTTTTGTCTGGATTAAGg |
| | 0511337.5 | taatttataaatttcatgttctacattttaaataatattttcttt |
| | | aaaaaaaatgagttccacaaaatcatggaatacttgaatttgaaa |
| | | ttcaagtgaccagccaaagctgctcaatatttactttgagacagg |
| | | gtctcactctgtcacccaggctggagtgcagtggtatgattacag |
| | | ctcattgcagcctcgacttcccaggcccaagcgatcctcccacct |
| | | tatcctccgaagtcactgggactacaggcatgtgccaccataccc |
| | | ggctaatttttaaattttttcgtagagacaaggtctcattatgtt |
| | | gcccaggctggttttgaactcctgggttcaagcaatcctcccacc |
| | | tcagcctcccaaagtgctgggattacaggcatgagccaccgtgcc |
| | | aggcctcatattttacatataaagtaaactattgagactcatgtg |
| | | atcattcctctcactgtcaatgacatacttctgctatctgaatta |
| | | gtgcaagatcagtccctataggttttgtttaacaaatgcagtaag |
| | | aggcctttcagtgtgttagctgggcctggggccccaggctgctaa |
| | | cagatgagatgaacaggtgaaggaaaaggaacttagagaaagaga |
| | | gggaaggagcaggtggagggaaggggagagttgctgcacttggaa |
| | | atgcttgctagaagggatcgcctcttttccaggtagaggctgtaa |
| | | gggaagctttacctagaattaaggttggaacagacactgcttcca |
| | | aatagttccttgctcactattttccttattgtcccaagatataat |
| | | gtgcatttccatgtgtgtgaaaggttatgacatttcatatacaac |
| | | aagcctcaattctggagatgcaggaaatttcaataattctcaggc |
| | | agcagctgccattcggtcaccagcacaggctctgattgtgctgtc |
| | | cagacagtaagtactagccacatgtgcctatttaaattcaaattt |
| | | aaattagttaagcttaaatacaattaaaaacgcagttccttggtc |
| | | ctactggccacacattaagtgttcaatggctactgtctaggacag |
| | | tggaaatgtagaacatttccatcatcacagaacgttctcttgaaa |
| | | agcactgttctggaaggtacttacccgttatgtacttttctgagt |
| | | tggtattcatacctagaagacctgaggtttatcacaagacataga |
| | | cttggaccaggcgcagtggctcatgcctgtaattccagcattttg |
| | | ggaggccgagataggtcccctgagcccaggagtctgataccagcc |
| | | tgggcaacatggcaaaacctcatctctactaaaaatacaaaaatt |
| | | agctgggggtggtggcacgtgcctgtagtcctagctacttaggag |
| | | gcttaggcgggaggattgcttgaatccagaaggcggagggtgcag |
| | | tgagccaagatcgcaccgctgcactccagcctgggcaacagagtg |
| | | agaccctgtctcaaaaaaaaaaaaaaaatgcatagactttatcct |
| | | gtatttctcatgctatttatttattgacatgcttgttcaagagaa |
| | | accatcactaaagcacaaaaccttgatcataacatagtaataata |
| | | atcaaacagcaaaaataataatagtaataagaatgttctgtggtg |
| | | atggaaatgttctatattttcattgtcctagacagtagccactaa |
| | | ccatgtataggcatggaacacttaacatgtggctagtaggaccaa |
| | | gggactgaatttttaattgtatttaatcttacttaatttaaattt |
| | | gaatttagatatccacacatgtttggatagcacagtcagagcctg |
| | | tgctggtgaaaggatggcaggtgctgcctgagaattactgaagtt |
| | | tccttgattattattagtttaataataataatcaagatagtaata |
| | | ataatcaagatagtaataataatcaagatctcagctgggcacagt |
| | | ggctcacgcactttgggaggctgaggcgggcagatcacctgacgt |
| | | cgggtttgagaccagcctggccaacatggtgaaaccctgtcccta |
| | | ctaaaaatacaaaaaaaaaaaattagctgggtgtggtggcacgtg |
| | | cctatgatcccagctacttgggaatctgaggcaggagaattgctt |
| | | gaacccaggaagcagaggttgcggtgagctgaatcatgccactgc |
| | | actccagcctgggcaacagagcagcacttcgtctcaaaaaaaaaa |
| | | aaaaaagatctcaaatgaattgggattgtattaagtaatgattaa |
| | | gtaatgtgattacagcaatcctcaagaaatatttcactgtggcca |
| | | gtaacaatgtgtaacagacctttaaacttctagagattttcctac |
| | | aacatgtgtctcaggctgatgtgttttatttagtgcttctcttgg |
| | | aaatgtcttgcccctcgatactttatcattaaggtctttaaggca |
| | | gggatcatgactctacttttttttttttttttttttttgggacgg |
| | | agtcttgctctgtcgcccaggctggagtgcagtggcacaatctta |
| | | gctcactgcaacctccgtctcctgggttcacgccattctcctgcc |
| | | tcagcctcccgagtagctgggactacaggcggctgccaccacgcc |
| | | cggctaattttttatatttttagtagagacggggtttcaccgtgt |
| | | tagccaggatggtctcgatctcctgacctcgtgatccacccacct |
| | | cggcctcccaaagtgctgggattacaggcttgagccaccacgccc |
| | | ggcctcatgactctacttctaatatctcatcatgtgctcttccac |
| | | tgaggcttctacttagagctacacaatctgggcagccatcctcag |
| | | tgccttatctaccaacatgctcaatatggctttgcagggttcact |
| | | gtctaccagcagggttcactatctaccaacatgctcaatatttct |
| | | ttgcagtcaggcagagcaggctttgcagttcaggcagggcagctg |
| | | gctgcaggccccagctgactcctggggatagaatgccaatatttc |
| | | agacattgcagagatttgaggcaatgtacataaagccctccacat |
| | | ataactgatgcacaataaatgacagttaatattatgcaacaagaa |
| | | tttcctggggggttttataattaatttttatttgtgtgaagtttt |
| | | ttccctcccttttactttaatcctttttggggggaaagcatcact |
| | | agtcacagttcacggcagcctcgacctcccaggctcaagcaaccc |
| | | tcccacctcagcctcctgagtagctgaaaccacaggtgtgtgcca |
| | | ccacacctgactaatttatttttattttctaatgaaacagaatct |
| | | tgccatattgcccaggctgatcttaaactcatgggctcaagcgat |
| | | cctcctgcctcagtcttccaaagtgccgggattatagatgtgagc |
| | | cactgcactcagcctttttttttttttttttaattgtagatagca |
| | | taaaacctactgttttaaccatgcttaagtgtacaattcagtggc |
| | | attaagtacattcacagtgttgtgcagccatcgccattatgctgc |
| | | attattttcagaactttttcattattctaaactgaaactttgtat |
| | | ccattgaacactaactcccaattcccccagtccctggtaacctcc |
| | | attctactttctgtcactgtgagtttgactattctaagtacctaa |
| | | tttaagtggaatcatacagtatttgtccttttgtgtcaggcttat |
| | | ttcactttgcatgatgttttcaaggttcatccatgttgtaacctg |
| | | tcagaatttaatttcttttcaggatgaaataatgttttattatat |
| | | acagtcacaccattttgtttatccattcatctattgatgtcttct |
| | | ctctcttacagCTCTTCAAATTCATCAATTTTAACAGGACCATGA |
| | | GCCAGCTCTCGACAACCATGTCTCGATGTGCCAAAGACCTGTTTG |
| | | GCTTTGCTATTATGTTCTTCATTATTTTCCTAGCGTATGCTCAGT |
| | | TGGCATACCTTGTCTTTGGCACTCAGGTCGATGACTTCAGTACTT |
| | | TCCAAGAGTGTATgtaagtatatatgaaattaagaagaaaaattt |
| | | aatcagagttgtcactgcttctcaagaataaatcttcatatgagg |
| | | ttgctatatgaccaccaattatttaaaaccagttattttaagtaa |
| | | gaattaattaccttttcccaaaacattgatctacccatgcaaaga |
| | | agacaatgcatcctgaaatgctgatgcttaagatagcagcccaaa |
| | | gtagtaaaatacagttaacagacataggaaaccaacactgttctg |
| | | aagactgagtttttctttgcaccaaatgcagatggtagcttctag |
| | | aaggctgtttgcctatattcttactcctgttgaatattgttgcca |
| | | tatatttagaacttcaagttattttctaaggaaaaaaacaagata |
| | | tttctaatattctaaggtaaactcagaccagtacaagaattttca |
| | | gtttttttttccaaagatcccaaatgtgaaataaaacaacaaaaa |
| | | gcagccagtgtcagatttctatgccatttagaaaggagttagttt |
| | | aaaaaggaatggaagtaatagggttttgtgcatagatatctcgaa |
| | | ttaatattgctgttgataaaagtgattttgctaagacccagcact |
| | | gacaacacttggccactttgatcccattttaagtacttgtcagaa |
| | | tattggatctttgaactcaaaccattttgggtttttggggttttt |
| | | ttgttttgtttttttttgttttgttttgtttttgaggcacggtct |
| | | tgctctgttgcccaggctggagtgcagtggtgcaatcatagctca |
| | | ctgcagccttgaactcctaggctcaagcaatcctgctgcctcagc |
| | | ctgctgagtagctgggactacaagtgtatgccaccatgcctggcc |
| | | aatttttaactttttttatgagaagggatctcactgtgtagccca |
| | | gggtggtattgaactccagggcctcacactgtcctctcacctcag |
| | | cttccaaaagtactgggattacaggcatgagccaccacaccaggc |
| | | cctgttgttttttttttaaagaaatttttaactttagaccgaggg |
| | | tgactgttgtcaaggtttagggttaagatgttttacctagattat |
| | | gtgttgaaatgttatagccaattgctttataagttattgaataat |
| | | aattgtattttctttttttttttttttttgagatggagtctcgtt |
| | | ccatcgcccaagctagagtgcagcggtgtaatctcagctcactgc |
| | | aacctctgcctcccgggttcaagcgattctcctgcctcagcctcc |
| | | cgaatagctgagattatgggcgcacgccaccaagcccagctaatt |
| | | tttgtatttttagtagagacggggtttcactatattggccaggct |
| | | gttctcgaactcctgacctcgtgatccgcccgcctcggcctccca |
| | | aagtgctgggattacagacgtgatccaccgtgcccagcttgtgtt |
| | | ttctttttaaccaaatggaaataacctctgtagcatgaaagcatt |
| | | ttattattattgcagaaggctttaattgctgatacaagtagcaag |
| | | actttgtaaatgggattgacaattttctgttattcggcagctacc |
| | | tatactgctaaaaggtccaaaaataatgaaatcatctttaagaaa |
| | | tgttgcatcaactagtggacattctttgtttttgtattgtggtgt |
| | | tttgttttatttttatagcttcactcaattccgtatcattttggg |
| | | cgatatcaactttgcagagattgaggaagctaatcgagttttggg |
| | | accaatttatttcactacatttgtgttctttatgttcttcattct |
| | | tttggtatgtacatttttatttatagtgaggttcaatttaaactt |
| | | cgtaaatccttgtcttctcttttctctcacactttatgtcctatc |
| | | aattttaaataaagacccaggaagtagaaaaaagtgtggatgttg |
| | | gaaaacttattttccttttattaattcacagttttgagactcata |
| | | tcaaatgtcttttctgtggtctttcattgatccatgtatatgtgt |
| | | ctattcaatgcaaaaaaaattagatctcttccatggtctttcatt |
| | | tctctctctatatatgtatctattccatgcaaaaaagaaattaga |
| | | tcaagtacaaatttataaagatacctaaaatagtgctttgcctaa |
| | | aaagtagaatatgcttacatgctttttaaactcatatgtcagcac |
| | | tttcgtagtcacttgctagcatgacttttctctctttcttctttt |
| | | ctttttaaaaaataagaacggaaaagcaagctagatctaagatgt |
| | | cgagtaatagttgagtgaatcattgcatgtcaaaattaggatatt |
| | | ctgttttaaattatttatatcccattcatctagagactgcctaca |
| | | gagaatattcaaataattaagtttaaaactaaatgtaacaatgaa |
| | | tggaaattgcattaaaattattttcaaaaataatttttttattct |
| | | cttgatttggtacaaatgaacatttttaatgtttttgccctaagt |
| | | caattaagtttttttaaggtgttttgttctttttcttaacattta |
| | | tatattcaattgtctactgagaaggtgttaagccagcttaattta |
| | | ggcaatatttttcatctaaacactaacagtcatcttaagaacaat |
| | | tttcttaagaaaataacattttttccatttcagtaaattgtgtaa |
| | | agatcccttgaggaaggttaagtgatcacattttcagtaattcag |
| | | tgtaataactctaaagtcagtccaggtattactggttaagtatat |
| | | ggtatttattgattgggtattagatgtactgtattaatttcctgt |
| | | ttaaaaaaaattttttttccggggagacacagcctctggtgtaaa |
| | | acaaaggtgtgttccctagctgtactttaacaggactgaaaaggt |
| | | caggaatatcattcaagttcatatgtatcttgctgtatgcatggt |
| | | ttatggctcatttttaaacttacacctcttaagcttcttcttcct |
| | | atcatatattaaaacaatggagagaagaataagcctctgttactc |
| | | taccattgatagtacttcggattctagagtacctgaatctctact |
| | | aagaaggcaaaaaccaggaattgagagtcctgcacctgacccttc |
| | | agttgatctcaggccacctagttttctccgtttatcaatctgcca |
| | | aacaaggatggatagagtcgtggcaactggaaaggctcaaatgtg |
| | | gaattgtttgaatgtggtcctttagtaggcagccatcttaccaga |
| | | tctagagtattcagtcatcttaccagatcagtcaccagaacatga |
| | | aaagaagctcttagtttctatctttatactaaaattgtttttttg |
| | | tacgactgcacaaaaaagaattgctctccttgcacctcccagaga |
| | | tataggtggatagatacatacatacgtacatacatacatacatac |
| | | atacatacatacatacatagacacatacatagatagaagtctact |
| | | ttcaatacaaacctgtcttttaaggaaatgacaagctgagcatag |
| | | ggttggccacctttctgagccgattgcctggtattagtttattgc |
| | | ccctgtttagcaagaaggcacagtgttaagaagtggctcagctga |
| | | accaggataaccccactcttcccccacatcaacaggaaagacatc |
| | | ctggtgcagatgtccatctgataattcagggaacctcgggagaca |
| | | ggatggagaggagggtgagctagcttcctcttcccacaccttcaa |
| | | gagcctttctcaagcactttctattttttgaaatctctttagagg |
| | | tcccagactttgatctgtttcaattaaggtattggcaggcattag |
| | | ttaacagccacttggaagcaaaaatagaacattagatccctgagt |
| | | tggaagagagaaggtagaaggtgttacttggactgcaattatctg |
| | | cacttggaattgagcatttagtcaaaaacttatatgtattctata |
| | | ttctattctcatttctgctacagaattgtaaacaatattcttcct |
| | | taatacagaaattcatagcccactaaaataagagcgttctcattt |
| | | gttcatttctcaatcatttaataagtatttactaagccactatat |
| | | ccatatatatatatatatcatatatactgtataatacacactgta |
| | | gtgttttttgtggattgtgtactatgaggtagtatgttagatact |
| | | gccagtactggggtaaggaaaacagcctgattaggcccttacgaa |
| | | gattcctcagacttgtggggaaaacagacattatcaaatagaaat |
| | | acttgcaaaccacagttatgtgttaaaaaggaaaaacaaagtaaa |
| | | aaaaaagttggtgggggggaacctgatctcctggatacagtgctt |
| | | cgagaaagtttgttgttggaaatgcaaaccattactactgtggaa |
| | | gggaaaggtcagaaaaatgaactcaccattactgaatagtaatag |
| | | tagctatcaattaggtggcacttacctgcatcaggacctgtcctg |
| | | agcactttacatagattgtctcactaaccagcccaacaaatatgt |
| | | aagggagatactattatttttcccattttattaatgtaaaacaat |
| | | taaataattctttaaaattagacttagaaaagtggagcaacaatc |
| | | ttagcagtgctaggactgaaatccaagtttgcttgactccaaagt |
| | | ctatctctcttccagaaactttttctttactatctgcctagtagg |
| | | cctgctgtattcctatttgcaacagccttttaaactctttaaaaa |
| | | tgtgtcctgtaaatttcatatatgattatacaaaaaaacttggaa |
| | | taagcatacaattctacttatctgtgttaactgttgaaatttgaa |
| | | gagctttttggaattctatacccttcagtagtgtatgtaaaagtt |
| | | tctaaatatagagaacatagataagcaaaaataatattaaataaa |
| | | ataatcgcaccattagtaggtaaatatactaatattttgttgtat |
| | | tttattcttgtatgttttcacaaagtatatcataaaatttttcct |
| | | gtggcatgacttaacggagaaaataatcttcccaaaacatgtggc |
| | | agcaaaactgttaatttattacatcaggctgggcacagtggctca |
| | | cgcttgcaatcccagcactttgggaagccgagggggcagatcact |
| | | tgaggccaggagttcgagaccagcctggccaacgtggtgaaacac |
| | | ctgttctactaaaaatacaacagttagccaggtgtagtggcacat |
| | | gcctgtaatcccagctactcaggaggctgagactcaagaattgct |
| | | tgaacccaggaggcagaggttgcagtaagctgaggtcgcgcccct |
| | | gcactccagcctgggcaacacagtgagactctgtctcaaaaaaaa |
| | | aaatttttttttaaataaataaataataaatttatgtcttcataa |
| | | agcactcagattaggaaaaaaaggataaacaaaaaggcatgtgtc |
| | | atttttttgattgataattccaaattatgtttcttcctttaattt |
| | | ttgccctcctttcatttacaaacagAATATGTTTTTGGCTATCAT |
| | | CAATGATACTTACTCTGAAGTGAAATCTGACTTGGCACAGCAGAA |
| | | AGCTGAAATGGAACTCTCAGATCTTATCAGAAAGgtaggaaaaac |
| | | cttaattctcagaattcttctgtttctgacataaaatgagcattg |
| | | tttcacccagattttcaaatcaacattgatccattgaaattgttt |
| | | gaaataaagaatacattgctatatttcaggaataatttaaatgtt |
| | | ccctatcttggagtcttgatggatatactgctatcttgaatttta |
| | | attctgggaatccttttatgccctggaattaaattctcaacaatc |
| | | ttttgacactttaagagctgagctgaaggttcatcaccttcatta |
| | | ttttgacatctcctgtagctggctctcacttcaggatcctgagtt |
| | | gagaataaactagaagggaagattatataaagggatttccacctc |
| | | ttctgtctcaattaccattttaaaaaaataaaaagttttagagga |
| | | aaacacttagtagttcaccctttacccttgaccttccacggcagt |
| | | tttaaaataagcaaaggaaaagattcatgaattcaggccatagcc |
| | | tggggcctgagaacttttacttatgcaccttctcaggaagggttt |
| | | cattgttaaatagaagggcaggacaggaaagttgggcctctttgt |
| | | tcttctcaatgtaacttctttatttggtttaaagtataaaatgta |
| | | tacaacaacaaataaccacatttaaaatacacagtttgtttccca |
| | | acatcattttgctaagtcatagtggctccttaactgtaatttttt |
| | | tttttattagtccaagccttaggattatgttatctgtgatatatg |
| | | ttataatagaaaacttaagcctcttaaaacaaagtccttgggatg |
| | | ggacctaagattcacattatcttgattccgcataacagttgctta |
| | | cattttagcaaatctccagtgtgtatgcaagcactcctcacttgg |
| | | cacaattctgatacacacaaactttggttaccccagttttgttat |
| | | gtaacaccaccttcaacaacacagttcaaatttcagttatcatag |
| | | tatattaactctgagtaacagcacaaagtacaaactccactgcta |
| | | gctcttcagtgtatagatcagttacctgagtaacagatgtgcagg |
| | | ctgagcaggctcactggtcagtcatgacactattttcagtctgtt |
| | | actaattggtcactgagcatctgctatccaattcacaaacaaaga |
| | | aagcatgtagtgttgcttccttgtgttccagtaataagcccatgt |
| | | gacattttacaaaaatggataattgaaaaagagaatgggtgcagt |
| | | ggctcacgcatgttgggaggccaaggcggatggatcacctgaggt |
| | | caggagttcgaaaccagcctggccaacatggtgaaaccccatctc |
| | | tactaaaaatacaaaattagctgagtgtggcgacagatgcctgta |
| | | atcccagctactggggaggctgaggcaggagaatcgcttgcacct |
| | | aggaggcggaggttgcagtgagccgagatcgtgccattgcactcc |
| | | agcctgggtaacaagagcgaaactccgtctcaaaaaaaaaaaaga |
| | | aaagaaaagaaaagaaaaagagaatgggctagcaaagaaatgaaa |
| | | aatggtaacactggaagtgaaaatcaaaacagagtaatggattta |
| | | tagaagaaatagctgagtgaagaagaaataggagtgttgacactg |
| | | cgatcattcaagagatccagatatggagccagaagaacttagggc |
| | | aggtctatcaacttaaatgaggaaaatagctgtgataaaacagat |
| | | gaagatgtcttgaggaaatgatgcctgcaaaaaacttcacattaa |
| | | gggaagtcttattagagatatttcacaataatgaaagtacaaaag |
| | | aaaaaatgttggggctggacatggtggcttactcctgtaatccca |
| | | gcactttgagaggccaaggtgggtggatcacttgaggccaggaag |
| | | tcgagacaagcctgatcaacatgatgaaaccccgtctctattaaa |
| | | aatacaaaaattagccagacatgatggtgcacacctgtaattcca |
| | | gctactcaagtggctgaggcacgagaattgcttgaaccagggagg |
| | | cggaggttgcagtaagctgagattgcaccactgcaatccagccag |
| | | gtgacagggtaagactgtgtctcaaaaataaataaaagaaaaata |
| | | tgttggaagctcatccacatttaagaaggaatatgacaattcact |
| | | aatgcatagaaaagaagttcactccacattgtaaagtgtacagtg |
| | | taatattatacaatgaaaacaaggcaagtgctgtttaaactactc |
| | | tggatacattttttacaaagaaataaaacactttagtttttaatg |
| | | tttctaatgttttacattttagtgtattaaatcaatattagtttt |
| | | cttcttttttaagctccctatacatttataactgacactaaggga |
| | | gtgtttaatgttttgattaaaagttgtaaagatcacagaacaatt |
| | | gtaattcttcccactgattattcagatcattttgcacaatttcag |
| | | cttgcatggtcacttacagtgccgcactatgtgcaaagcaaggtc |
| | | aggtctaaagttcgctaatgaaaaatcctcggccaggggcagtgg |
| | | ctcacccctgtaatcctagcactttggaaggcgaggcaggcagat |
| | | cgcttgagctcaggagttcaacaccagcctgggcaacatggtgag |
| | | accctgtctctacaaaaaaaaaaaaatagcaaggcgtggtgactc |
| | | acacctgtagtcccagctacttgttgggggctgagttgggaaaat |
| | | cacttgagctcaggaggtcgaggctgcagtgagccagaatcacgc |
| | | cactgcccttctgcctgggtgacagagtaagatcctgtctcaaaa |
| | | aaagggaaaatcctcatctacatttcactgggttttttgtttgtt |
| | | tgtttgtttgtttatacacacttaaggaaattactgtctagaaga |
| | | tagataatataaaaaataaaaatgcaattcatgattcgggtttct |
| | | tggtattcctaagaactgttgcacagtactttatgctctgaggca |
| | | gacagctatagcatatatagtaatttttgtttctatcacataaac |
| | | ttgaatacacatatgagtaaaagacctttagttcttcatgactta |
| | | ctgaaagaccctgactttttccatgtaactgttccacaagtgttt |
| | | tatggaaaactggatacattaattcttcattcatccagcacgtac |
| | | ttgttgaatggccaatgtacaccaggtttgtagtagttactactg |
| | | tgaatggaaagtaaaacagatgcaaaaggagaatacactaaacca |
| | | agtcttttattttttctctctctgatagGGCTACCATAAAGCTTT |
| | | GGTCAAACTAAAACTGAAAAAAAATACCGTGGATGACATTTCAGA |
| | | GAGTCTGCGGCAAGGAGGAGGCAAGTTAAACTTTGACGAACTTCG |
| | | ACAAGATCTCAAAGGgtgagaatcatgcttcctgaggttctgaaa |
| | | aattcctgcttctaaagataaattcctggtgataagagtatttct |
| | | agcccaagggctcatacagatactttttttttttttttccagagg |
| | | caggtatctttctggaacatgttataagaggaaaacttgccccca |
| | | tttggtgatttctcctttcctcctgcattttgatgtctctgtgtt |
| | | gagggtgaactgggtacaaggaatgatttttatctgtatcctctc |
| | | tctaatttcagGAAGGGCCATACTGATGCAGAGATTGAGGCAATA |
| | | TTCACAAAGTACGACCAAGATGGAGACCAAGAACTGACCGAACAT |
| | | GAACATCAGCAGATGAGAGACGACTTGGAGAAAGAGAGGgtgggt |
| | | ctggtttaggagaaccggatttgatttggtacctacaacaccaca |
| | | gatgtatcaaacactatagaagtagtgggttattgagtctcttgc |
| | | ccattccccaccacactctctctctctctcagtcggtttatgtgt |
| | | tagtaccctgtttattccagaaagaatatataacacaattatgta |
| | | taaaaatgggtggttagcatgatataaaaacgtcaaaatgaaaag |
| | | caagcaaaacaaaagtaaaaataatggattattaatgaagcttaa |
| | | aaatgcattcataaaaacacatatgcttattaagattgggctaca |
| | | aattgggccctaagcttgctggtaatcagcttgaaaagagaagcc |
| | | tgattagctgcagagtccacaatgtccgtgagagtgaagaaaaca |
| | | aaaaatgacttaccaagagatgtgaaattattctggttagttagt |
| | | ggctatttaaattgttaacttttttttcttttttttttttttttg |
| | | agatggagtcttgctctgcctcccaggctggagtgcagtggcaca |
| | | atcgcgactcactgcaacctccacctcccgggttcaagcgattct |
| | | cttgcctcagcctcccaagtagctaggactacaggcacatgccat |
| | | catgcccggctaatttttgtatttgtagtagatatggggtttcac |
| | | catgttggtctcaaactcctgactgcaagcaatctgcccaccttg |
| | | gcctcccaaagtggtgggattacaggcagtagccaccgtgccttt |
| | | cctaaattattaacatttataataaaattaacagccgccttccat |
| | | ttgaatactttttacaaaatagttaaaaataaacataagtgggct |
| | | tttatagtcagaaaaaaaaattcaaagctttaccattaactttca |
| | | aaaataaatggttagacagcaacaacaaaaatctgtggtaactga |
| | | ggtacagagaacacagatgaatgttattacaaaagccactttcct |
| | | atgagaagtctaggacagtggtttctaaatgccactccacagaca |
| | | gtgctagtaggtgacagacttctccagtcacagtgaaatttaagc |
| | | ataaagaaaatgaggaaaatttttacaaggctctatttagacaaa |
| | | gttcttattctgacattacatctttcctactttggagctgttgaa |
| | | tgtattatcttttatgaaaagaaggcgatccaggttgagcatccc |
| | | taacccaaatatgtgagtctgaaatgctccaaaacctaaaacttc |
| | | ttgagcacaaacatgatagtcaaaggtcatgcttaaaggaaatgc |
| | | tgtcattggagcagtttggattttgggttttcagattagggatgc |
| | | tgaaccagtaagtataatgcaaacattccaaaatatttttgaaaa |
| | | tcccaaatccaaaacacttctgatcccaagtatttcaaataaggg |
| | | atactcaacctgtaatatatttcttcatttctttatttattttat |
| | | tattattttaagatggctcatggcccactgcagcctcaaactcct |
| | | aggctcaagtgatcttccgacctcaacctcccaggtagctcaggt |
| | | agctgggactgcaggcatgcatcaccatgcctggctaatttttta |
| | | aaaaattttttgtggaggcagagtctcaccttgctgcccaggcca |
| | | gtctcaaactcctggcttcaagcagtactcctgcctcagcctccc |
| | | aaagtattaggattacaggtgtgaccactatgcctggcccatatt |
| | | tcttcatttagttttttctttgcctgctgtgtttttaatgttctt |
| | | tcttgttcaaacaaaaagttggctattccttgctgttagttaaat |
| | | ttgccaatctatgaaactgaaaaatgcaggagtcccagcctggtg |
| | | ttaaatacaaagaaatcccaggtaaatggcatgcacccagttcct |
| | | gcttgcccaagtccttggtgaggcttctgtggggtctcagtgttc |
| | | tgctcctcactcagtgaccccttgttcttcagGAGGACCTGGATT |
| | | TGGATCACAGTTCTTTACCACGTCCCATGAGCAGCCGAAGTTTCC |
| | | CTCGAAGCCTGGATGACTCTGAGGAGGATGACGATGAAGATAGCG |
| | | GACATAGCTCCAGAAGGAGGGGAAGCATTTCTAGTGGCGTTTCTT |
| | | ACGAAGAGTTTCAAGTgtaagtataaaggaattggcagaatttgc |
| | | gttgacaagagtccacatgagaccaggcagttccctcatctctct |
| | | gaattcactcctttccattactaatcatccagcttttaaaaataa |
| | | cttatactggccagacgcagtggctcatgcctgtaatcccaccac |
| | | tttgggaggccaaagcaggcaggtcatgaggtcagaagttcgaga |
| | | cgagcctggccaacatagtgaaaccccatctctattaaaaataca |
| | | aaaaattagctgggcatggtggtgggcacctgtaatcctagctac |
| | | ttgggaggctgaggcaagagaattgcttgaacccgggaggcggag |
| | | gttacagagagccgagatggctccactgcacaccagcctgggcga |
| | | cagtgcaagactctgtctcaaaaaaaaaaaaacttgtcaattggt |
| | | gttttgtttcttacataatatgtttactataaaaattagcaaata |
| | | agagcaaaagaaaacattaacatttcacatatttctaccaatgaa |
| | | aaatgtttattaatatatcagtgtttgtgtctctatttgcatgtg |
| | | tttatcaatgtttctatatatttttatggcctaacatatggttcg |
| | | tcctgaagaatgttccttgtgcatttgagaagaatgaatattctg |
| | | ctgttcaagtgttctgtagatgtttgttaggtctagtttgtttac |
| | | agttttattcaggtctcccatttcctggttgatcttagatgtgcc |
| | | tagatgtggtattcacgtttgaaagtggggtattaaagtctccaa |
| | | gtattattagttggagttcatcccttcaattctgtaaggttttgc |
| | | tttgtgtattttggaactctgttgttgggtgcatacatatttata |
| | | actagtatattttcctaatatattgaccctattttctctctcaac |
| | | ttaatgaggctaaagaaaaaaaagaattgaccctgttttcattac |
| | | aagatgttatccactttatctctagtaaaattctttgttttaagt |
| | | attttttgtttgatattactgtaaccactccagctttcttttggt |
| | | tgctgtttgcatgataaatctttttccatccttttactttaaact |
| | | tatttatatctttcagtctgaagtatgtctctcctgtagacagca |
| | | tataattggatcttatctttttatccagtttgacaatttctgttt |
| | | ttgattagattgcttaatccattcatttaatgttatcattgatgt |
| | | agttgatttctgtctgctattttattttttgttttctagcttact |
| | | tttttttgttcctctttcactgctttcttgtacattaagtgaata |
| | | ttttcaagtataacatttaaatttttttaatgatttttcattttt |
| | | tttagtcaggagttgctctaagacttagtttatacaattaagtta |
| | | tgaaaaattacttcagatatatattaactgaatccagtgagatat |
| | | agaaatcatttctatttagcttttttcctcttccctctttttgtg |
| | | ctatatattcatctatctatatgtatatatagtcatctacatatg |
| | | ttgcaaatcacattgttagaacaatgttacatttttataacacac |
| | | tgtgtaatatatagtatataattttatatctcttaatgaagctga |
| | | gagaagaggaagtatatatttataaatgtgtatattaacctactt |
| | | ttttaccatttctaattctcttcttttgctcctctggattcaagt |
| | | tatcatctgtcgtcattctctttctccgatacagctttgctactg |
| | | cctacctcctattattgtcaaatatattacatttctattatagac |
| | | cttcagatccaattatgtacatatttttacacaactgctttttaa |
| | | atcagttaaggaacaaaaggagaaatgtacatatatactatattt |
| | | tatacctacacagttatctttaccagtgttctttgccttttcatg |
| | | tggattctgattactatctggagtcacttgctttcagcataaaga |
| | | atttcctttagtattttttgtaaagcaggtttgctagcaatgaat |
| | | tctttcattttttgtttatctgagaatgtttttctttctccttca |
| | | tttcctctggcttgtattgtttctgatgagaagacaggtgctaat |
| | | tttactgtggtccccttgtacatgatgactcaattttctctcacc |
| | | actttcaagatttttttgcttttgtctttcattatttttactgtg |
| | | atatgtctgggtataaatctctgagttcatcctacttagaaagtg |
| | | ttttttctgcttctttcactttctcttctcctttgggacccgcat |
| | | tatgcatatgcttaggggtatcacatatttctcttaggctctgtt |
| | | cgtcgtcattttttttccctctctgttcctcagagtgcatagtct |
| | | gtattgatgtatcttcaagttcactgacttgttcttctgtcagct |
| | | tgcttaaatctctgttgagctcctctagttatttatttatttatt |
| | | tatttattttatatatatatatatatatatatatatagagagaga |
| | | gagagagagagagagagaaagagagagagagagagacagggagac |
| | | agggtctcactccatcacccaggctggagtacagtggtgtaatca |
| | | tggctctctacagcctgaacacctgggctcaaatgatcctcctgc |
| | | ctcaacctcccaagtagctaggactatgggcacatgctgccatgc |
| | | ctggctaatttttaaaaaaaaatttgtagagatggcatcttgtta |
| | | tgttgcccaggttggtctcaaactcccggcctcaagtgatccttc |
| | | cgcctcggcctcccaaagtgctggggtcacaggtatgagccaccg |
| | | cacttagcctgaattttttatttattatacttttcaactccagaa |
| | | tttctatttggttctttttagcaatctctgtctctttattctgta |
| | | tttgatgatatcctgtatttgatgagacaatgtcatcataacttc |
| | | cttttttttttaagagatagggtctctctctgtcacccaggctag |
| | | agtgcagtggcatgatcctagctcactgcagcctcgaactcctga |
| | | actcaagcaatcctcccacctcagcctcctaagtacctgacacta |
| | | caggcatgagccactgtacccagctaatttttattttttgtagag |
| | | atggggtctaagttgcccaggctggtctcaaactcctgggctcaa |
| | | gtgatcctccctgctcaggctcccaaagtgctgggattacaggca |
| | | tgaaccactggacccagcctcctttatttctttaaccatggtggg |
| | | gttttttattatttgtttgtttgttttaacctgtttgaatatatt |
| | | tataataggtaccttcaggtctttgtctgctaagtctgacatctg |
| | | ggccctctcaaagacagtttcagttgtctttttttttttcttctt |
| | | gtgtatgggaacattttcctgtttttttgttttgttttttgttgt |
| | | tttgttttgttgttgttgttgttgttaaagattggacatgttaga |
| | | taatatattgtaggaactctggctagtgattccctttcctcccca |
| | | ggacttgtttttatttctctttgcttgtttactttttcatacagt |
| | | ctgtttccccacagtgtctgcctctgactttattccttagagggc |
| | | gcagctgtggccatgtacgtagtcactctgggaagacagtggttt |
| | | tagcagcgtgctcattaactttctctgacctctttgttatacctc |
| | | ctgcctctgtggatattagacccagttattacatttcattgttta |
| | | ctgattggtctattgttttccagaatgccttgggatgtaatttgc |
| | | tccacagtctgatccacttaaattcaggcctctttgcagggctag |
| | | ttttagaggccagtctttttttttttttttttttttttttgagac |
| | | agagtctcactctgttgcccaggccggaatgtagtggtgcagtct |
| | | cagctcactgcagcctcaatctctcaggctcaagcaatcttccca |
| | | cctcagcttctcaagtagctaggactacaggtgcacgccaccatg |
| | | cctggttaatttttgcattttttatagagatggggttttaccatg |
| | | ttgccccggctggtctcaaactcctgagttcaagcaatccaccca |
| | | cctcagcctcacaaaaggccgggattacagatatgcaccaccacg |
| | | cctggcccctgaggccagtctttaactgtcttcttagctgtctct |
| | | ttccctggttctctctggtgaactagctggtaatttgtttatctc |
| | | ataaggctaccagattccttgtaaatgcttatccccacaatctcc |
| | | attgttttcaagagcatcgttagtctttaatttcctcacgcttca |
| | | ttccaaataaagtccattcacttgagaagagttctatattcctat |
| | | gacctgtgtctccccatgagcaaaactgctactgctttacagagc |
| | | cagggacagtggcccacctctctgtggcatcctgctttatgaaca |
| | | agtcactgggctcagatggcagtctctgattttctcaccttgctt |
| | | cttctggcatggaaactccaccctacaagtgggaactgagtggaa |
| | | gaagagagccccattccccttagccactcttaacaggattagaac |
| | | ctctgcaacatgcatttaagaatgggaacaggctgggcacagtgg |
| | | ctcacgcctgtaatcctagcactttgggaggtcaaggcaggagga |
| | | ttgcttgagccaggagttcaggaccagcctgtgcaacatggtgag |
| | | accctcatctctacaagaaatagaaaaattaactggtgggttgtg |
| | | tatacctgtaatcccagctactcgggagcctgaagtgggaggatt |
| | | gcttgaacctggaggcagaggttgcagtgagtcaagattacacca |
| | | ttgcacgccagcctgggcaacagagtgagactctgtctcaaaaaa |
| | | aaaaaaaaaaaaagagtaggaacattgaggctgggcatggtggct |
| | | catgcctgtaatcctagcactatgggaggccaaggcaggagtatc |
| | | acttgacgctaggagttcaagaccaacctgggtaacatagcgaga |
| | | ctttgtctctattaaaaaaaaaaaaaaaaaaggtgtaaaaaactt |
| | | ttgtaacaagagtgggaaagccgggcacagtggctcacacctgta |
| | | atcccagcactttggaaggccaaggcaggcaggcggatcacctga |
| | | ggtcaggagtttgagaccagcctggccaacgtggtgaaaccccat |
| | | ctctactaaaaaatagaaaaattatctgggcatggtggtgcacac |
| | | ctgtagtcccagctactcgggaggctgaggcaggagaatcacttg |
| | | aacctacgaggcagaagttgcagtaagccaagatcacgccactgc |
| | | actccagcctgggcgacagagcaagactctgtctcaaaaaaaaaa |
| | | aaagagtggaaatgttaggatgagaaatgctggcagcctgcccct |
| | | ccctgggagatactgtagccctagactggaagttgggggaggagg |
| | | gagccctgtgctcttagctgcacccatgtgaagttgtgcttctat |
| | | cacatgagctggggacaggagagaaggctcagattatggcttcag |
| | | tgccacagaatctcttcatactaaaatttagtagattttcttgaa |
| | | taaatgctttttcatttgctgtacacccttaaaagtttctagaaa |
| | | tttttaatatttgagttttaaaaaataattttcaacagttacagt |
| | | tatttcactaaagagagagtctacagaaccctcttgccaccattg |
| | | cagaggttgtctttggcttacgagtttttaaagtatttgtataca |
| | | ttttttaagttcaaaataatagaattgtaagtgaacatgctgttt |
| | | tcatactgtttttcaagctttatttaatatattgtaaatctaatt |
| | | ctattttattaaatagtctgccacagtataatgtctgatgtctcc |
| | | ttagaattttattgtatggatgaacaatgattatttaatttccta |
| | | ccaattgttgggtgttttttgtttgtttgtttgtttttgagactg |
| | | ggtctcactctgtcacccaggctggagtgcaggagtgcggtggaa |
| | | tgatcacggctcactgcagcctcaacatcccaaggctcaggtgat |
| | | ccttccacctcagcctcgcaagtagctgggagtacaggcacatgc |
| | | caccatgcccacctattttttagagatgaagttttgccatcctgc |
| | | ccaggctggctcgaactcctggcctcaagcgatctgcacacttcc |
| | | gcctcccaaaatgccaggattacaggcgtgagccatcatgcccta |
| | | cccccccatcaattgtttgatgtagccatttttcaatgatccgcg |
| | | attaagaagcagcactcttttatagccaaaaattacacatatata |
| | | aaattttcctttagaaaatgttctaaaaatggaatgtctaactaa |
| | | agggttaggcatacattcttaagacttctgatacgcgctgacttg |
| | | caggaaagttgtttcagttaacactcctaccagcggcatccgaga |
| | | gttaatctgtaaagcttgagacaacttagaaagtgtttcaaatga |
| | | ttgtgttgcttaagaaaaaaatcttagcacttccttttgaaaagc |
| | | cagtggggctgaaaagacaatgacaagcactttgtccctctgtac |
| | | tgtgttttccttgcagCCTGGTGAGACGAGTGGACCGGATGGAGC |
| | | ATTCCATCGGCAGCATAGTGTCCAAGATTGACGCCGTGATCGTGA |
| | | AGCTAGAGATTATGGAGCGAGCCAAACTGAAGAGGAGGGAGGTGC |
| | | TGGGAAGGCTGTTGGATGGGGTGGCCGAGgtcagtagtcatgagc |
| | | tgaaaacaccgctgctgagcatggtgttattaatgaaaatatatg |
| | | ttgctgacagttgtatttgaagtattgaagaagagtaaaaaaaat |
| | | ttacgtttatagaaattcacaatgatgtttccatttactctcatt |
| | | ttcagatttttttctctgaaacagaaacactctttctataaaatc |
| | | tcttgctataaaacatcaatgtagtcatattgtctaacccttagg |
| | | ctgagatgtttatctttctccataactacagataaaattataatc |
| | | tggaggtgttactttcttaatactccatatgctaatggtcctgcc |
| | | ttcactgcagggtagaattaagtgaaaaattactccagcaactct |
| | | gagatttgctattatatgctgtaaatctccagccttaccaaacta |
| | | cagattatttggtccctggacttcctaaggcatttccttctactg |
| | | cccccaacaccagtttctttttccctttttagGATGAAAGGCTGG |
| | | GTCGTGACAGTGAAATCCATAGGGAACAGATGGAACGGCTAGTAC |
| | | GTGAAGAGTTGGAACGCTGGGAATCCGATGATGCAGCTTCCCAGA |
| | | TCAGTCATGGTTTAGGCACGCCAGTGGGACTAAATGGTCAACCTC |
| | | GCCCCAGAAGCTCCCGCCCATCTTCCTCCCAATCTACAGAAGGCA |
| | | TGGAAGGTGCAGGTGGAAATGGGAGTTCTAATGTCCACGTATGAT |
| | | ATGTGTGTTTCAGTATGTGTGTTTCTAATAAGTGAGGAAGTGGCT |
| | | GTCCTGAATTGCTGTAACAAGCACACTATTTATATGCCCTGACCA |
| | | CCATAGGATGCTAGTCTTTGTGACCGATTGCTAATCTTCTGCACT |
| | | TTAATTTATTTTATATAAACTTTACCCATGGTTCAAAGATTTTTT |
| | | TTTCTTTTTCTCATATAAGAAA (SEQ ID NO: 242) |
| | premrna | ACAGTTCAACAATATAGCTGCTGTCACAGTATTTTTTGTCTGGAT |
| | ENST0000 | TAAGgtaatttataaatttcatgttctacattttaaataatattt |
| | 0512858.1 | tctttaaaaaaaatgagttccacaaaatcatggaatacttgaatt |
| | | tgaaattcaagtgaccagccaaagctgctcaatatttactttgag |
| | | acagggtctcactctgtcacccaggctggagtgcagtggtatgat |
| | | tacagctcattgcagcctcgacttcccaggcccaagcgatcctcc |
| | | caccttatcctccgaagtcactgggactacaggcatgtgccacca |
| | | tacccggctaatttttaaattttttcgtagagacaaggtctcatt |
| | | atgttgcccaggctggttttgaactcctgggttcaagcaatcctc |
| | | ccacctcagcctcccaaagtgctgggattacaggcatgagccacc |
| | | gtgccaggcctcatattttacatataaagtaaactattgagactc |
| | | atgtgatcattcctctcactgtcaatgacatacttctgctatctg |
| | | aattagtgcaagatcagtccctataggttttgtttaacaaatgca |
| | | gtaagaggcctttcagtgtgttagctgggcctggggccccaggct |
| | | gctaacagatgagatgaacaggtgaaggaaaaggaacttagagaa |
| | | agagagggaaggagcaggtggagggaaggggagagttgctgcact |
| | | tggaaatgcttgctagaagggatcgcctcttttccaggtagaggc |
| | | tgtaagggaagctttacctagaattaaggttggaacagacactgc |
| | | ttccaaatagttccttgctcactattttccttattgtcccaagat |
| | | ataatgtgcatttccatgtgtgtgaaaggttatgacatttcatat |
| | | acaacaagcctcaattctggagatgcaggaaatttcaataattct |
| | | caggcagcagctgccattcggtcaccagcacaggctctgattgtg |
| | | ctgtccagacagtaagtactagccacatgtgcctatttaaattca |
| | | aatttaaattagttaagcttaaatacaattaaaaacgcagttcct |
| | | tggtcctactggccacacattaagtgttcaatggctactgtctag |
| | | gacagtggaaatgtagaacatttccatcatcacagaacgttctct |
| | | tgaaaagcactgttctggaaggtacttacccgttatgtacttttc |
| | | tgagttggtattcatacctagaagacctgaggtttatcacaagac |
| | | atagacttggaccaggcgcagtggctcatgcctgtaattccagca |
| | | ttttgggaggccgagataggtcccctgagcccaggagtctgatac |
| | | cagcctgggcaacatggcaaaacctcatctctactaaaaatacaa |
| | | aaattagctgggggtggtggcacgtgcctgtagtcctagctactt |
| | | aggaggcttaggcgggaggattgcttgaatccagaaggcggaggg |
| | | tgcagtgagccaagatcgcaccgctgcactccagcctgggcaaca |
| | | gagtgagaccctgtctcaaaaaaaaaaaaaaaatgcatagacttt |
| | | atcctgtatttctcatgctatttatttattgacatgcttgttcaa |
| | | gagaaaccatcactaaagcacaaaaccttgatcataacatagtaa |
| | | taataatcaaacagcaaaaataataatagtaataagaatgttctg |
| | | tggtgatggaaatgttctatattttcattgtcctagacagtagcc |
| | | actaaccatgtataggcatggaacacttaacatgtggctagtagg |
| | | accaagggactgaatttttaattgtatttaatcttacttaattta |
| | | aatttgaatttagatatccacacatgtttggatagcacagtcaga |
| | | gcctgtgctggtgaaaggatggcaggtgctgcctgagaattactg |
| | | aagtttccttgattattattagtttaataataataatcaagatag |
| | | taataataatcaagatagtaataataatcaagatctcagctgggc |
| | | acagtggctcacgcactttgggaggctgaggcgggcagatcacct |
| | | gacgtcgggtttgagaccagcctggccaacatggtgaaaccctgt |
| | | ccctactaaaaatacaaaaaaaaaaaattagctgggtgtggtggc |
| | | acgtgcctatgatcccagctacttgggaatctgaggcaggagaat |
| | | tgcttgaacccaggaagcagaggttgcggtgagctgaatcatgcc |
| | | actgcactccagcctgggcaacagagcagcacttcgtctcaaaaa |
| | | aaaaaaaaaaagatctcaaatgaattgggattgtattaagtaatg |
| | | attaagtaatgtgattacagcaatcctcaagaaatatttcactgt |
| | | ggccagtaacaatgtgtaacagacctttaaacttctagagatttt |
| | | cctacaacatgtgtctcaggctgatgtgttttatttagtgcttct |
| | | cttggaaatgtcttgcccctcgatactttatcattaaggtcttta |
| | | aggcagggatcatgactctacttttttttttttttttttttttgg |
| | | gacggagtcttgctctgtcgcccaggctggagtgcagtggcacaa |
| | | tcttagctcactgcaacctccgtctcctgggttcacgccattctc |
| | | ctgcctcagcctcccgagtagctgggactacaggcggctgccacc |
| | | acgcccggctaattttttatatttttagtagagacggggtttcac |
| | | cgtgttagccaggatggtctcgatctcctgacctcgtgatccacc |
| | | cacctcggcctcccaaagtgctgggattacaggcttgagccacca |
| | | cgcccggcctcatgactctacttctaatatctcatcatgtgctct |
| | | tccactgaggcttctacttagagctacacaatctgggcagccatc |
| | | ctcagtgccttatctaccaacatgctcaatatggctttgcagggt |
| | | tcactgtctaccagcagggttcactatctaccaacatgctcaata |
| | | tttctttgcagtcaggcagagcaggctttgcagttcaggcagggc |
| | | agctggctgcaggccccagctgactcctggggatagaatgccaat |
| | | atttcagacattgcagagatttgaggcaatgtacataaagccctc |
| | | cacatataactgatgcacaataaatgacagttaatattatgcaac |
| | | aagaatttcctggggggttttataattaatttttatttgtgtgaa |
| | | gttttttccctcccttttactttaatcctttttggggggaaagca |
| | | tcactagtcacagttcacggcagcctcgacctcccaggctcaagc |
| | | aaccctcccacctcagcctcctgagtagctgaaaccacaggtgtg |
| | | tgccaccacacctgactaatttatttttattttctaatgaaacag |
| | | aatcttgccatattgcccaggctgatcttaaactcatgggctcaa |
| | | gcgatcctcctgcctcagtcttccaaagtgccgggattatagatg |
| | | tgagccactgcactcagcctttttttttttttttttaattgtaga |
| | | tagcataaaacctactgttttaaccatgcttaagtgtacaattca |
| | | gtggcattaagtacattcacagtgttgtgcagccatcgccattat |
| | | gctgcattattttcagaactttttcattattctaaactgaaactt |
| | | tgtatccattgaacactaactcccaattcccccagtccctggtaa |
| | | cctccattctactttctgtcactgtgagtttgactattctaagta |
| | | cctaatttaagtggaatcatacagtatttgtccttttgtgtcagg |
| | | cttatttcactttgcatgatgttttcaaggttcatccatgttgta |
| | | acctgtcagaatttaatttcttttcaggatgaaataatgttttat |
| | | tatatacagtcacaccattttgtttatccattcatctattgatgt |
| | | cttctctctcttacagCTCTTCAAATTCATCAATTTTAACAGGAC |
| | | CATGAGCCAGCTCTCGACAACCATGTCTCGATGTGCCAAAGACCT |
| | | GTTTGGCTTTGCTATTATGTTCTTCATTATTTTCCTAGCGTATGC |
| | | TCAGTTGGCATACCTTGTCTTTGGCACTCAGGTCGATGACTTCAG |
| | | TACTTTCCAAGAGTGTATgtaagtatatatgaaattaagaagaaa |
| | | aatttaatcagagttgtcactgcttctcaagaataaatcttcata |
| | | tgaggttgctatatgaccaccaattatttaaaaccagttatttta |
| | | agtaagaattaattaccttttcccaaaacattgatctacccatgc |
| | | aaagaagacaatgcatcctgaaatgctgatgcttaagatagcagc |
| | | ccaaagtagtaaaatacagttaacagacataggaaaccaacactg |
| | | ttctgaagactgagtttttctttgcaccaaatgcagatggtagct |
| | | tctagaaggctgtttgcctatattcttactcctgttgaatattgt |
| | | tgccatatatttagaacttcaagttattttctaaggaaaaaaaca |
| | | agatatttctaatattctaaggtaaactcagaccagtacaagaat |
| | | tttcagtttttttttccaaagatcccaaatgtgaaataaaacaac |
| | | aaaaagcagccagtgtcagatttctatgccatttagaaaggagtt |
| | | agtttaaaaaggaatggaagtaatagggttttgtgcatagatatc |
| | | tcgaattaatattgctgttgataaaagtgattttgctaagaccca |
| | | gcactgacaacacttggccactttgatcccattttaagtacttgt |
| | | cagaatattggatctttgaactcaaaccattttgggtttttgggg |
| | | tttttttgttttgtttttttttgttttgttttgtttttgaggcac |
| | | ggtcttgctctgttgcccaggctggagtgcagtggtgcaatcata |
| | | gctcactgcagccttgaactcctaggctcaagcaatcctgctgcc |
| | | tcagcctgctgagtagctgggactacaagtgtatgccaccatgcc |
| | | tggccaatttttaactttttttatgagaagggatctcactgtgta |
| | | gcccagggtggtattgaactccagggcctcacactgtcctctcac |
| | | ctcagcttccaaaagtactgggattacaggcatgagccaccacac |
| | | caggccctgttgttttttttttaaagaaatttttaactttagacc |
| | | gagggtgactgttgtcaaggtttagggttaagatgttttacctag |
| | | attatgtgttgaaatgttatagccaattgctttataagttattga |
| | | ataataattgtattttctttttttttttttttttgagatggagtc |
| | | tcgttccatcgcccaagctagagtgcagcggtgtaatctcagctc |
| | | actgcaacctctgcctcccgggttcaagcgattctcctgcctcag |
| | | cctcccgaatagctgagattatgggcgcacgccaccaagcccagc |
| | | taatttttgtatttttagtagagacggggtttcactatattggcc |
| | | aggctgttctcgaactcctgacctcgtgatccgcccgcctcggcc |
| | | tcccaaagtgctgggattacagacgtgatccaccgtgcccagctt |
| | | gtgttttctttttaaccaaatggaaataacctctgtagcatgaaa |
| | | gcattttattattattgcagaaggctttaattgctgatacaagta |
| | | gcaagactttgtaaatgggattgacaattttctgttattcggcag |
| | | ctacctatactgctaaaaggtccaaaaataatgaaatcatcttta |
| | | agaaatgttgcatcaactagtggacattctttgtttttgtattgt |
| | | ggtgttttgttttatttttatagcttcactcaattccgtatcatt |
| | | ttgggcgatatcaactttgcagagattgaggaagctaatcgagtt |
| | | ttgggaccaatttatttcactacatttgtgttctttatgttcttc |
| | | attcttttggtatgtacatttttatttatagtgaggttcaattta |
| | | aacttcgtaaatccttgtcttctcttttctctcacactttatgtc |
| | | ctatcaattttaaataaagacccaggaagtagaaaaaagtgtgga |
| | | tgttggaaaacttattttccttttattaattcacagttttgagac |
| | | tcatatcaaatgtcttttctgtggtctttcattgatccatgtata |
| | | tgtgtctattcaatgcaaaaaaaattagatctcttccatggtctt |
| | | tcatttctctctctatatatgtatctattccatgcaaaaaagaaa |
| | | ttagatcaagtacaaatttataaagatacctaaaatagtgctttg |
| | | cctaaaaagtagaatatgcttacatgctttttaaactcatatgtc |
| | | agcactttcgtagtcacttgctagcatgacttttctctctttctt |
| | | cttttctttttaaaaaataagaacggaaaagcaagctagatctaa |
| | | gatgtcgagtaatagttgagtgaatcattgcatgtcaaaattagg |
| | | atattctgttttaaattatttatatcccattcatctagagactgc |
| | | ctacagagaatattcaaataattaagtttaaaactaaatgtaaca |
| | | atgaatggaaattgcattaaaattattttcaaaaataattttttt |
| | | attctcttgatttggtacaaatgaacatttttaatgtttttgccc |
| | | taagtcaattaagtttttttaaggtgttttgttctttttcttaac |
| | | atttatatattcaattgtctactgagaaggtgttaagccagctta |
| | | atttaggcaatatttttcatctaaacactaacagtcatcttaaga |
| | | acaattttcttaagaaaataacattttttccatttcagtaaattg |
| | | tgtaaagatcccttgaggaaggttaagtgatcacattttcagtaa |
| | | ttcagtgtaataactctaaagtcagtccaggtattactggttaag |
| | | tatatggtatttattgattgggtattagatgtactgtattaattt |
| | | cctgtttaaaaaaaattttttttccggggagacacagcctctggt |
| | | gtaaaacaaaggtgtgttccctagctgtactttaacaggactgaa |
| | | aaggtcaggaatatcattcaagttcatatgtatcttgctgtatgc |
| | | atggtttatggctcatttttaaacttacacctcttaagcttcttc |
| | | ttcctatcatatattaaaacaatggagagaagaataagcctctgt |
| | | tactctaccattgatagtacttcggattctagagtacctgaatct |
| | | ctactaagaaggcaaaaaccaggaattgagagtcctgcacctgac |
| | | ccttcagttgatctcaggccacctagttttctccgtttatcaatc |
| | | tgccaaacaaggatggatagagtcgtggcaactggaaaggctcaa |
| | | atgtggaattgtttgaatgtggtcctttagtaggcagccatctta |
| | | ccagatctagagtattcagtcatcttaccagatcagtcaccagaa |
| | | catgaaaagaagctcttagtttctatctttatactaaaattgttt |
| | | ttttgtacgactgcacaaaaaagaattgctctccttgcacctccc |
| | | agagatataggtggatagatacatacatacgtacatacatacata |
| | | catacatacatacatacatacatagacacatacatagatagaagt |
| | | ctactttcaatacaaacctgtcttttaaggaaatgacaagctgag |
| | | catagggttggccacctttctgagccgattgcctggtattagttt |
| | | attgcccctgtttagcaagaaggcacagtgttaagaagtggctca |
| | | gctgaaccaggataaccccactcttcccccacatcaacaggaaag |
| | | acatcctggtgcagatgtccatctgataattcagggaacctcggg |
| | | agacaggatggagaggagggtgagctagcttcctcttcccacacc |
| | | ttcaagagcctttctcaagcactttctattttttgaaatctcttt |
| | | agaggtcccagactttgatctgtttcaattaaggtattggcaggc |
| | | attagttaacagccacttggaagcaaaaatagaacattagatccc |
| | | tgagttggaagagagaaggtagaaggtgttacttggactgcaatt |
| | | atctgcacttggaattgagcatttagtcaaaaacttatatgtatt |
| | | ctatattctattctcatttctgctacagaattgtaaacaatattc |
| | | ttccttaatacagaaattcatagcccactaaaataagagcgttct |
| | | catttgttcatttctcaatcatttaataagtatttactaagccac |
| | | tatatccatatatatatatatatcatatatactgtataatacaca |
| | | ctgtagtgttttttgtggattgtgtactatgaggtagtatgttag |
| | | atactgccagtactggggtaaggaaaacagcctgattaggccctt |
| | | acgaagattcctcagacttgtggggaaaacagacattatcaaata |
| | | gaaatacttgcaaaccacagttatgtgttaaaaaggaaaaacaaa |
| | | gtaaaaaaaaagttggtgggggggaacctgatctcctggatacag |
| | | tgcttcgagaaagtttgttgttggaaatgcaaaccattactactg |
| | | tggaagggaaaggtcagaaaaatgaactcaccattactgaatagt |
| | | aatagtagctatcaattaggtggcacttacctgcatcaggacctg |
| | | tcctgagcactttacatagattgtctcactaaccagcccaacaaa |
| | | tatgtaagggagatactattatttttcccattttattaatgtaaa |
| | | acaattaaataattctttaaaattagacttagaaaagtggagcaa |
| | | caatcttagcagtgctaggactgaaatccaagtttgcttgactcc |
| | | aaagtctatctctcttccagaaactttttctttactatctgccta |
| | | gtaggcctgctgtattcctatttgcaacagccttttaaactcttt |
| | | aaaaatgtgtcctgtaaatttcatatatgattatacaaaaaaact |
| | | tggaataagcatacaattctacttatctgtgttaactgttgaaat |
| | | ttgaagagctttttggaattctatacccttcagtagtgtatgtaa |
| | | aagtttctaaatatagagaacatagataagcaaaaataatattaa |
| | | ataaaataatcgcaccattagtaggtaaatatactaatattttgt |
| | | tgtattttattcttgtatgttttcacaaagtatatcataaaattt |
| | | ttcctgtggcatgacttaacggagaaaataatcttcccaaaacat |
| | | gtggcagcaaaactgttaatttattacatcaggctgggcacagtg |
| | | gctcacgcttgcaatcccagcactttgggaagccgaggcgggcag |
| | | atcacttgaggccaggagttcgagaccagcctggccaacgtggtg |
| | | aaacactgtctctactaaaaatacaacagttagccaggtgtagtg |
| | | gcacatgcctgtaatcccagctactcaggaggctgagactcaaga |
| | | attgcttgaacccaggaggcagaggttgcagtaagctgaggtcgc |
| | | gcccctgcactccagcctgggcaacacagtgagactctgtctcaa |
| | | aaaaaaaaatttttttttaaataaataaataataaatttatgtct |
| | | tcataaagcactcagattaggaaaaaaaggataaacaaaaaggca |
| | | tgtgtcatttttttgattgataattccaaattatgtttcttcctt |
| | | taatttttgccctcctttcatttacaaacagAATATGTTTTTGGC |
| | | TATCATCAATGATACTTACTCTGAAGTGAAATCTGACTTGGCACA |
| | | GCAGAAAGCTGAAATGGAACTCTCAGATCTTATCAGAAAGgtagg |
| | | aaaaaccttaattctcagaattcttctgtttctgacataaaatga |
| | | gcattgtttcacccagattttcaaatcaacattgatccattgaaa |
| | | ttgtttgaaataaagaatacattgctatatttcaggaataattta |
| | | aatgttccctatcttggagtcttgatggatatactgctatcttga |
| | | attttaattctgggaatccttttatgccctggaattaaattctca |
| | | acaatcttttgacactttaagagctgagctgaaggttcatcacct |
| | | tcattattttgacatctcctgtagctggctctcacttcaggatcc |
| | | tgagttgagaataaactagaagggaagattatataaagggatttc |
| | | cacctcttctgtctcaattaccattttaaaaaaataaaaagtttt |
| | | agaggaaaacacttagtagttcaccctttacccttgaccttccac |
| | | ggcagttttaaaataagcaaaggaaaagattcatgaattcaggcc |
| | | atagcctggggcctgagaacttttacttatgcaccttctcaggaa |
| | | gggtttcattgttaaatagaagggcaggacaggaaagttgggcct |
| | | ctttgttcttctcaatgtaacttctttatttggtttaaagtataa |
| | | aatgtatacaacaacaaataaccacatttaaaatacacagtttgt |
| | | ttcccaacatcattttgctaagtcatagtggctccttaactgtaa |
| | | tttttttttttattagtccaagccttaggattatgttatctgtga |
| | | tatatgttataatagaaaacttaagcctcttaaaacaaagtcctt |
| | | gggatgggacctaagattcacattatcttgattccgcataacagt |
| | | tgcttacattttagcaaatctccagtgtgtatgcaagcactcctc |
| | | acttggcacaattctgatacacacaaactttggttaccccagttt |
| | | tgttatgtaacaccaccttcaacaacacagttcaaatttcagtta |
| | | tcatagtatattaactctgagtaacagcacaaagtacaaactcca |
| | | ctgctagctcttcagtgtatagatcagttacctgagtaacagatg |
| | | tgcaggctgagcaggctcactggtcagtcatgacactattttcag |
| | | tctgttactaattggtcactgagcatctgctatccaattcacaaa |
| | | caaagaaagcatgtagtgttgcttccttgtgttccagtaataagc |
| | | ccatgtgacattttacaaaaatggataattgaaaaagagaatggg |
| | | tgcagtggctcacgcatgttgggaggccaaggcggatggatcacc |
| | | tgaggtcaggagttcgaaaccagcctggccaacatggtgaaaccc |
| | | catctctactaaaaatacaaaattagctgagtgtggcgacagatg |
| | | cctgtaatcccagctactggggaggctgaggcaggagaatcgctt |
| | | gcacctaggaggcggaggttgcagtgagccgagatcgtgccattg |
| | | cactccagcctgggtaacaagagcgaaactccgtctcaaaaaaaa |
| | | aaaagaaaagaaaagaaaagaaaaagagaatgggctagcaaagaa |
| | | atgaaaaatggtaacactggaagtgaaaatcaaaacagagtaatg |
| | | gatttatagaagaaatagctgagtgaagaagaaataggagtgttg |
| | | acactgcgatcattcaagagatccagatatggagccagaagaact |
| | | tagggcaggtctatcaacttaaatgaggaaaatagctgtgataaa |
| | | acagatgaagatgtcttgaggaaatgatgcctgcaaaaaacttca |
| | | cattaagggaagtcttattagagatatttcacaataatgaaagta |
| | | caaaagaaaaaatgttggggctggacatggtggcttactcctgta |
| | | atcccagcactttgagaggccaaggtgggtggatcacttgaggcc |
| | | aggaagtcgagacaagcctgatcaacatgatgaaaccccgtctct |
| | | attaaaaatacaaaaattagccagacatgatggtgcacacctgta |
| | | attccagctactcaagtggctgaggcacgagaattgcttgaacca |
| | | gggaggcggaggttgcagtaagctgagattgcaccactgcaatcc |
| | | agccaggtgacagggtaagactgtgtctcaaaaataaataaaaga |
| | | aaaatatgttggaagctcatccacatttaagaaggaatatgacaa |
| | | ttcactaatgcatagaaaagaagttcactccacattgtaaagtgt |
| | | acagtgtaatattatacaatgaaaacaaggcaagtgctgtttaaa |
| | | ctactctggatacattttttacaaagaaataaaacactttagttt |
| | | ttaatgtttctaatgttttacattttagtgtattaaatcaatatt |
| | | agttttcttcttttttaagctccctatacatttataactgacact |
| | | aagggagtgtttaatgttttgattaaaagttgtaaagatcacaga |
| | | acaattgtaattcttcccactgattattcagatcattttgcacaa |
| | | tttcagcttgcatggtcacttacagtgccgcactatgtgcaaagc |
| | | aaggtcaggtctaaagttcgctaatgaaaaatcctcggccagggg |
| | | cagtggctcacccctgtaatcctagcactttggaaggcgaggcag |
| | | gcagatcgcttgagctcaggagttcaacaccagcctgggcaacat |
| | | ggtgagaccctgtctctacaaaaaaaaaaaaatagcaaggcgtgg |
| | | tgactcacacctgtagtcccagctacttgttgggggctgagttgg |
| | | gaaaatcacttgagctcaggaggtcgaggctgcagtgagccagaa |
| | | tcacgccactgcccttctgcctgggtgacagagtaagatcctgtc |
| | | tcaaaaaaagggaaaatcctcatctacatttcactgggttttttg |
| | | tttgtttgtttgtttgtttatacacacttaaggaaattactgtct |
| | | agaagatagataatataaaaaataaaaatgcaattcatgattcgg |
| | | gtttcttggtattcctaagaactgttgcacagtactttatgctct |
| | | gaggcagacagctatagcatatatagtaatttttgtttctatcac |
| | | ataaacttgaatacacatatgagtaaaagacctttagttcttcat |
| | | gacttactgaaagaccctgactttttccatgtaactgttccacaa |
| | | gtgttttatggaaaactggatacattaattcttcattcatccagc |
| | | acgtacttgttgaatggccaatgtacaccaggtttgtagtagtta |
| | | ctactgtgaatggaaagtaaaacagatgcaaaaggagaatacact |
| | | aaaccaagtcttttattttttctctctctgatagGGCTACCATAA |
| | | AGCTTTGGTCAAACTAAAACTGAAAAAAAATACCGTGGATGACAT |
| | | TTCAGAGAGTCTGCGGCAAGGAGGAGGCAAGTTAAACTTTGACGA |
| | | ACTTCGACAAGATCTCAAAGGGTGAGAATCATGCTTCCTGAGGTT |
| | | ACTGAAAATTCCTGCTTCTAAAGATAAATTCCTGGTGATAAGAGT |
| | | ATTTCTAGCCCAAGGGCTCATACAGATACTTTTTTTTTTTTTTTC |
| | | CAGAGGCAGGTATCTTTCTGGAACATGTTATAAGAGGAAAACTTG |
| | | CCCCCATTTGGTGATTTCTCCTTTCCTCCTGCATTTTGATGTCTC |
| | | TGTGTTGAGGGTGAACTGGGTACAAGGAATGATTTTTATCTGTAT |
| | | CCTCTCTCTAATTTCAGGAAGGGCCATACTGATGCAGAGATTGAG |
| | | GCAATATTCACAAAGTACGACCAAGATGGAGACCAAGAACTGACC |
| | | GAACATGAACATCAGCAGATGAGAGACGACTTGGAGAAAGAGAGG |
| | | gtgggtctggtttaggagaaccggatttgatttggtacctacaac |
| | | accacagatgtatcaaacactatagaagtagtgggttattgagtc |
| | | tcttgcccattccccaccacactctctctctctctcagtcggttt |
| | | atgtgttagtaccctgtttattccagaaagaatatataacacaat |
| | | tatgtataaaaatgggtggttagcatgatataaaaacgtcaaaat |
| | | gaaaagcaagcaaaacaaaagtaaaaataatggattattaatgaa |
| | | gcttaaaaatgcattcataaaaacacatatgcttattaagattgg |
| | | gctacaaattgggccctaagcttgctggtaatcagcttgaaaaga |
| | | gaagcctgattagctgcagagtccacaatgtccgtgagagtgaag |
| | | aaaacaaaaaatgacttaccaagagatgtgaaattattctggtta |
| | | gttagtggctatttaaattgttaacttttttttcttttttttttt |
| | | tttttgagatggagtcttgctctgcctcccaggctggagtgcagt |
| | | ggcacaatcgcgactcactgcaacctccacctcccgggttcaagc |
| | | gattctcttgcctcagcctcccaagtagctaggactacaggcaca |
| | | tgccatcatgcccggctaatttttgtatttgtagtagatatgggg |
| | | tttcaccatgttggtctcaaactcctgactgcaagcaatctgccc |
| | | accttggcctcccaaagtggtgggattacaggcagtagccaccgt |
| | | gcctttcctaaattattaacatttataataaaattaacagccgcc |
| | | ttccatttgaatactttttacaaaatagttaaaaataaacataag |
| | | tgggcttttatagtcagaaaaaaaaattcaaagctttaccattaa |
| | | ctttcaaaaataaatggttagacagcaacaacaaaaatctgtggt |
| | | aactgaggtacagagaacacagatgaatgttattacaaaagccac |
| | | tttcctatgagaagtctaggacagtggtttctaaatgccactcca |
| | | cagacagtgctagtaggtgacagacttctccagtcacagtgaaat |
| | | ttaagcataaagaaaatgaggaaaatttttacaaggctctattta |
| | | gacaaagttcttattctgacattacatctttcctactttggagct |
| | | gttgaatgtattatcttttatgaaaagaaggcgatccaggttgag |
| | | catccctaacccaaatatgtgagtctgaaatgctccaaaacctaa |
| | | aacttcttgagcacaaacatgatagtcaaaggtcatgcttaaagg |
| | | aaatgctgtcattggagcagtttggattttgggttttcagattag |
| | | ggatgctgaaccagtaagtataatgcaaacattccaaaatatttt |
| | | tgaaaatcccaaatccaaaacacttctgatcccaagtatttcaaa |
| | | taagggatactcaacctgtaatatatttcttcatttctttattta |
| | | ttttattattattttaagatggctcatggcccactgcagcctcaa |
| | | actcctaggctcaagtgatcttccgacctcaacctcccaggtagc |
| | | tcaggtagctgggactgcaggcatgcatcaccatgcctggctaat |
| | | tttttaaaaaattttttgtggaggcagagtctcaccttgctgccc |
| | | aggccagtctcaaactcctggcttcaagcagtactcctgcctcag |
| | | cctcccaaagtattaggattacaggtgtgaccactatgcctggcc |
| | | catatttcttcatttagttttttctttgcctgctgtgtttttaat |
| | | gttctttcttgttcaaacaaaaagttggctattccttgctgttag |
| | | ttaaatttgccaatctatgaaactgaaaaatgcaggagtcccagc |
| | | ctggtgttaaatacaaagaaatcccaggtaaatggcatgcaccca |
| | | gttcctgcttgcccaagtccttggtgaggcttctgtggggtctca |
| | | gtgttctgctcctcactcagtgaccccttgttcttcagGAGGACC |
| | | TGGATTTGGATCACAGTTCTTTACCACGTCCCATGAGCAGCCGAA |
| | | GTTTCCCTCGAAGCCTGGATGACTCTGAGGAGGATGACGATGAAG |
| | | ATAGCGGACATAGCTCCAGAAGGAGGGGAAGCATTTCTAGTGGCG |
| | | TTTCTTACGAAGAGTTTCAAGTgtaagtataaaggaattggcaga |
| | | atttgcgttgacaagagtccacatgagaccaggcagttccctcat |
| | | ctctctgaattcactcctttccattactaatcatccagcttttaa |
| | | aaataacttatactggccagacgcagtggctcatgcctgtaatcc |
| | | caccactttgggaggccaaagcaggcaggtcatgaggtcagaagt |
| | | tcgagacgagcctggccaacatagtgaaaccccatctctattaaa |
| | | aatacaaaaaattagctgggcatggtggtgggcacctgtaatcct |
| | | agctacttgggaggctgaggcaagagaattgcttgaacccgggag |
| | | gcggaggttacagagagccgagatggctccactgcacaccagcct |
| | | gggcgacagtgcaagactctgtctcaaaaaaaaaaaaacttgtca |
| | | attggtgttttgtttcttacataatatgtttactataaaaattag |
| | | caaataagagcaaaagaaaacattaacatttcacatatttctacc |
| | | aatgaaaaatgtttattaatatatcagtgtttgtgtctctatttg |
| | | catgtgtttatcaatgtttctatatatttttatggcctaacatat |
| | | ggttcgtcctgaagaatgttccttgtgcatttgagaagaatgaat |
| | | attctgctgttcaagtgttctgtagatgtttgttaggtctagttt |
| | | gtttacagttttattcaggtctcccatttcctggttgatcttaga |
| | | tgtgcctagatgtggtattcacgtttgaaagtggggtattaaagt |
| | | ctccaagtattattagttggagttcatcccttcaattctgtaagg |
| | | ttttgctttgtgtattttggaactctgttgttgggtgcatacata |
| | | tttataactagtatattttcctaatatattgaccctattttctct |
| | | ctcaacttaatgaggctaaagaaaaaaaagaattgaccctgtttt |
| | | cattacaagatgttatccactttatctctagtaaaattctttgtt |
| | | ttaagtattttttgtttgatattactgtaaccactccagctttct |
| | | tttggttgctgtttgcatgataaatctttttccatccttttactt |
| | | taaacttatttatatctttcagtctgaagtatgtctctcctgtag |
| | | acagcatataattggatcttatctttttatccagtttgacaattt |
| | | ctgtttttgattagattgcttaatccattcatttaatgttatcat |
| | | tgatgtagttgatttctgtctgctattttattttttgttttctag |
| | | cttacttttttttgttcctctttcactgctttcttgtacattaag |
| | | tgaatattttcaagtataacatttaaatttttttaatgatttttc |
| | | attttttttagtcaggagttgctctaagacttagtttatacaatt |
| | | aagttatgaaaaattacttcagatatatattaactgaatccagtg |
| | | agatatagaaatcatttctatttagcttttttcctcttccctctt |
| | | tttgtgctatatattcatctatctatatgtatatatagtcatcta |
| | | catatgttgcaaatcacattgttagaacaatgttacatttttata |
| | | acacactgtgtaatatatagtatataattttatatctcttaatga |
| | | agctgagagaagaggaagtatatatttataaatgtgtatattaac |
| | | ctacttttttaccatttctaattctcttcttttgctcctctggat |
| | | tcaagttatcatctgtcgtcattctctttctccgatacagctttg |
| | | ctactgcctacctcctattattgtcaaatatattacatttctatt |
| | | atagaccttcagatccaattatgtacatatttttacacaactgct |
| | | ttttaaatcagttaaggaacaaaaggagaaatgtacatatatact |
| | | atattttatacctacacagttatctttaccagtgttctttgcctt |
| | | ttcatgtggattctgattactatctggagtcacttgctttcagca |
| | | taaagaatttcctttagtattttttgtaaagcaggtttgctagca |
| | | atgaattctttcattttttgtttatctgagaatgtttttctttct |
| | | ccttcatttcctctggcttgtattgtttctgatgagaagacaggt |
| | | gctaattttactgtggtccccttgtacatgatgactcaattttct |
| | | ctcaccactttcaagatttttttgcttttgtctttcattattttt |
| | | actgtgatatgtctgggtataaatctctgagttcatcctacttag |
| | | aaagtgttttttctgcttctttcactttctcttctcctttgggac |
| | | ccgcattatgcatatgcttaggggtatcacatatttctcttaggc |
| | | tctgttcgtcgtcattttttttccctctctgttcctcagagtgca |
| | | tagtctgtattgatgtatcttcaagttcactgacttgttcttctg |
| | | tcagcttgcttaaatctctgttgagctcctctagttatttattta |
| | | tttatttatttattttatatatatatatatatatatatatataga |
| | | gagagagagagagagagagagagaaagagagagagagagagacag |
| | | ggagacagggtctcactccatcacccaggctggagtacagtggtg |
| | | taatcatggctctctacagcctgaacacctgggctcaaatgatcc |
| | | tcctgcctcaacctcccaagtagctaggactatgggcacatgctg |
| | | ccatgcctggctaatttttaaaaaaaaatttgtagagatggcatc |
| | | ttgttatgttgcccaggttggtctcaaactcccggcctcaagtga |
| | | tccttccgcctcggcctcccaaagtgctggggtcacaggtatgag |
| | | ccaccgcacttagcctgaattttttatttattatacttttcaact |
| | | ccagaatttctatttggttctttttagcaatctctgtctctttat |
| | | tctgtatttgatgatatcctgtatttgatgagacaatgtcatcat |
| | | aacttccttttttttttaagagatagggtctctctctgtcaccca |
| | | ggctagagtgcagtggcatgatcctagctcactgcagcctcgaac |
| | | tcctgaactcaagcaatcctcccacctcagcctcctaagtacctg |
| | | acactacaggcatgagccactgtacccagctaatttttatttttt |
| | | gtagagatggggtctaagttgcccaggctggtctcaaactcctgg |
| | | gctcaagtgatcctccctgctcaggctcccaaagtgctgggatta |
| | | caggcatgaaccactggacccagcctcctttatttctttaaccat |
| | | ggtggggttttttattatttgtttgtttgttttaacctgtttgaa |
| | | tatatttataataggtaccttcaggtctttgtctgctaagtctga |
| | | catctgggccctctcaaagacagtttcagttgtcttttttttttt |
| | | cttcttgtgtatgggaacattttcctgtttttttgttttgttttt |
| | | tgttgttttgttttgttgttgttgttgttgttaaagattggacat |
| | | gttagataatatattgtaggaactctggctagtgattccctttcc |
| | | tccccaggacttgtttttatttctctttgcttgtttactttttca |
| | | tacagtctgtttccccacagtgtctgcctctgactttattcctta |
| | | gagggcgcagctgtggccatgtacgtagtcactctgggaagacag |
| | | tggttttagcagcgtgctcattaactttctctgacctctttgtta |
| | | tacctcctgcctctgtggatattagacccagttattacatttcat |
| | | tgtttactgattggtctattgttttccagaatgccttgggatgta |
| | | atttgctccacagtctgatccacttaaattcaggcctctttgcag |
| | | ggctagttttagaggccagtctttttttttttttttttttttttt |
| | | tgagacagagtctcactctgttgcccaggccggaatgtagtggtg |
| | | cagtctcagctcactgcagcctcaatctctcaggctcaagcaatc |
| | | ttcccacctcagcttctcaagtagctaggactacaggtgcacgcc |
| | | accatgcctggttaatttttgcattttttatagagatggggtttt |
| | | accatgttgccccggctggtctcaaactcctgagttcaagcaatc |
| | | cacccacctcagcctcacaaaaggccgggattacagatatgcacc |
| | | accacgcctggcccctgaggccagtctttaactgtcttcttagct |
| | | gtctctttccctggttctctctggtgaactagctggtaatttgtt |
| | | tatctcataaggctaccagattccttgtaaatgcttatccccaca |
| | | atctccattgttttcaagagcatcgttagtctttaatttcctcac |
| | | gcttcattccaaataaagtccattcacttgagaagagttctatat |
| | | tcctatgacctgtgtctccccatgagcaaaactgctactgcttta |
| | | cagagccagggacagtggcccacctctctgtggcatcctgcttta |
| | | tgaacaagtcactgggctcagatggcagtctctgattttctcacc |
| | | ttgcttcttctggcatggaaactccaccctacaagtgggaactga |
| | | gtggaagaagagagccccattccccttagccactcttaacaggat |
| | | tagaacctctgcaacatgcatttaagaatgggaacaggctgggca |
| | | cagtggctcacgcctgtaatcctagcactttgggaggtcaaggca |
| | | ggaggattgcttgagccaggagttcaggaccagcctgtgcaacat |
| | | ggtgagaccctcatctctacaagaaatagaaaaattaactggtgg |
| | | gttgtgtatacctgtaatcccagctactcgggagcctgaagggga |
| | | ggattgcttgaacctggaggcagaggttgcagtgagtcaagatta |
| | | caccattgcacgccagcctgggcaacagagtgagactctgtctca |
| | | aaaaaaaaaaaaaaaaaagagtaggaacattgaggctgggcatgg |
| | | tggctcatgcctgtaatcctagcactatgggaggccaaggcagga |
| | | gtatcacttgacgctaggagttcaagaccaacctgggtaacatag |
| | | cgagactttgtctctattaaaaaaaaaaaaaaaaaaggtgtaaaa |
| | | aacttttgtaacaagagtgggaaagccgggcacagtggctcacac |
| | | ctgtaatcccagcactttggaaggccaaggcaggcaggcggatca |
| | | cctgaggtcaggagtttgagaccagcctggccaacgtggtgaaac |
| | | cccatctctactaaaaaatagaaaaattatctgggcatggtggtg |
| | | cacacctgtagtcccagctactcgggaggctgaggcaggagaatc |
| | | acttgaacctacgaggcagaagttgcagtaagccaagatcacgcc |
| | | actgcactccagcctgggcgacagagcaagactctgtctcaaaaa |
| | | aaaaaaaagagtggaaatgttaggatgagaaatgctggcagcctg |
| | | cccctccctgggagatactgtagccctagactggaagttggggga |
| | | ggagggagccctgtgctcttagctgcacccatgtgaagttgtgct |
| | | tctatcacatgagctggggacaggagagaaggctcagattatggc |
| | | ttcagtgccacagaatctcttcatactaaaatttagtagattttc |
| | | ttgaataaatgctttttcatttgctgtacacccttaaaagtttct |
| | | agaaatttttaatatttgagttttaaaaaataattttcaacagtt |
| | | acagttatttcactaaagagagagtctacagaaccctcttgccac |
| | | cattgcagaggttgtctttggcttacgagtttttaaagtatttgt |
| | | atacattttttaagttcaaaataatagaattgtaagtgaacatgc |
| | | tgttttcatactgtttttcaagctttatttaatatattgtaaatc |
| | | taattctattttattaaatagtctgccacagtataatgtctgatg |
| | | tctccttagaattttattgtatggatgaacaatgattatttaatt |
| | | tcctaccaattgttgggtgttttttgtttgtttgtttgtttttga |
| | | gactgggtctcactctgtcacccaggctggagtgcaggagtgcgg |
| | | tggaatgatcacggctcactgcagcctcaacatcccaaggctcag |
| | | gtgatccttccacctcagcctcgcaagtagctgggagtacaggca |
| | | catgccaccatgcccacctattttttagagatgaagttttgccat |
| | | cctgcccaggctggctcgaactcctggcctcaagcgatctgcaca |
| | | cttccgcctcccaaaatgccaggattacaggcgtgagccatcatg |
| | | ccctacccccccatcaattgtttgatgtagccatttttcaatgat |
| | | ccgcgattaagaagcagcactcttttatagccaaaaattacacat |
| | | atataaaattttcctttagaaaatgttctaaaaatggaatgtcta |
| | | actaaagggttaggcatacattcttaagacttctgatacgcgctg |
| | | acttgcaggaaagttgtttcagttaacactcctaccagcggcatc |
| | | cgagagttaatctgtaaagcttgagacaacttagaaagtgtttca |
| | | aatgattgtgttgcttaagaaaaaaatcttagcacttccttttga |
| | | aaagccagtggggctgaaaagacaatgacaagcactttgtccctc |
| | | tgtactgtgttttccttgcagCCTGGTGAGACGAGTGGACCGGAT |
| | | GGAGCATTCCATCGGCAGCATAGTGTCCAAGATTGACGCCGTGAT |
| | | CGTGAAGCTAGAGATTATGGAGCGAGCCAAACTGAAGAGGAGGGA |
| | | GGTGCTGGGAAGGCTGTTGGATGGGGTGGCCGAGgtcagtagtca |
| | | tgagctgaaaacaccgctgctgagcatggtgttattaatgaaaat |
| | | atatgttgctgacagttgtatttgaagtattgaagaagagtaaaa |
| | | aaaatttacgtttatagaaattcacaatgatgtttccatttactc |
| | | tcattttcagatttttttctctgaaacagaaacactctttctata |
| | | aaatctcttgctataaaacatcaatgtagtcatattgtctaaccc |
| | | ttaggctgagatgtttatctttctccataactacagataaaatta |
| | | taatctggaggtgttactttcttaatactccatatgctaatggtc |
| | | ctgccttcactgcagggtagaattaagtgaaaaattactccagca |
| | | actctgagatttgctattatatgctgtaaatctccagccttacca |
| | | aactacagattatttggtccctggacttcctaaggcatttcctta |
| | | ctgcccccaacaccagtttctttttcccttttttcagGATGAAAG |
| | | GCTGGGTCGTGACAGTGAAATCCATAGGGAACAGATGGAACGGCT |
| | | AGTACGTGAAGAGTTGGAACGCTGGGAATCCGATGATGCAGCTTC |
| | | CCAGATCAGTCATGGTTTAGGCACGCCAGTGGGACTAAATGGTCA |
| | | ACCTCGCCCCAGAAGCTCCCGCCCATCTTCCTCCCAATCTACAGA |
| | | AGGCATGGAAGGTGCAGGTGGAAATGGGAGTTCTAATGTCCACGT |
| | | ATGATATGTGTGTTTCAGTATGTGTGTTTCTAATAAGTGAGGAAG |
| | | TGGCTGTCCTGAATTGCTGTAACAAGCACACTATTTATATGCCCT |
| | | GACCACCATAGGATGCTAGTCTTTGTGACCGATTGCTAATCTTCT |
| | | GCACTTTAATTTATTTTATATAAACTTTACCCATGGTT |
| | | (SEQ ID NO: 243) |
| transcript | transcript | AGGCGGCGGCGGGCGCCGGGAAGAAAGGAACATGGCTCCTGAG |
| | ENST0000 | GCGCACAGCGCCGAGCGCGGCGCCGCGCACCCGCGCGCCGGAC |
| | 0237596.7 | GCCAGTGACCGCGATGGTGAACTCCAGTCGCGTGCAGCCTCAGC |
| | | AGCCCGGGGACGCCAAGCGGCCGCCCGCGCCCCGCGCGCCGGA |
| | | CCCGGGCCGGCTGATGGCTGGCTGCGCGGCCGTGGGCGCCAGCC |
| | | TCGCCGCCCCGGGCGGCCTCTGCGAGCAGCGGGGCCTGGAGATC |
| | | GAGATGCAGCGCATCCGGCAGGCGGCCGCGCGGGACCCCCCGG |
| | | CCGGAGCCGCGGCCTCCCCTTCTCCTCCGCTCTCGTCGTGCTCC |
| | | CGGCAGGCGTGGAGCCGCGATAACCCCGGCTTCGAGGCCGAGGA |
| | | GGAGGAGGAGGAGGTGGAAGGGGAAGAAGGCGGAATGGTGGT |
| | | GGAGATGGACGTAGAGTGGCGCCCGGGCAGCCGGAGGTCGGCC |
| | | GCCTCCTCGGCCGTGAGCTCCGTGGGCGCGCGGAGCCGGGGGCT |
| | | TGGGGGCTACCACGGCGCGGGCCACCCGAGCGGGAGGCGGCGC |
| | | CGGCGAGAGGACCAGGGCCCGCCGTGCCCCAGCCCAGTCGGCG |
| | | GCGGGGACCCGCTGCATCGCCACCTCCCCCTGGAAGGGCAGCCG |
| | | CCCCGAGTGGCCTGGGCGGAGAGGCTGGTTCGCGGGCTGCGAGG |
| | | TCTCTGGGGAACAAGACTCATGGAGGAAAGCAGCACTAACCGA |
| | | GAGAAATACCTTAAAAGTGTTTTACGGGAACTGGTCACATACCT |
| | | CCTTTTTCTCATAGTCTTGTGCATCTTGACCTACGGCATGATGA |
| | | GCTCCAATGTGTACTACTACACCCGGATGATGTCACAGCTCTTC |
| | | CTAGACACCCCCGTGTCCAAAACGGAGAAAACTAACTTTAAAAC |
| | | TCTGTCTTCCATGGAAGACTTCTGGAAGTTCACAGAAGGCTCCT |
| | | TATTGGATGGGCTGTACTGGAAGATGCAGCCCAGCAACCAGACT |
| | | GAAGCTGACAACCGAAGTTTCATCTTCTATGAGAACCTGCTGTT |
| | | AGGGGTTCCACGAATACGGCAACTCCGAGTCAGAAATGGATCCT |
| | | GCTCTATCCCCCAGGACTTGAGAGATGAAATTAAAGAGTGCTAT |
| | | CCGAAGATGTCTACTCTGTCAGTAGTGAAGATAGGGCTCCCTTT |
| | | GGGCCATGGAACCGCTTGGATCTACACAAGTGAAAAAGACTTGA |
| | | ATGGTAGTAGCCACTGGGGAATCATTGCAACTTATAGTGGAGCT |
| | | GGCTATTATCTGGATTTGTCAAGAACAAGAGAGGAAACAGCTGC |
| | | ACAAGTTGCTAGCCTCAAGAAAAATGTCTGGCTGGACCGAGGAA |
| | | CCAGGGCAACTTTTATTGACTTCTCAGTGTACAACGCCAACATT |
| | | AACCTGTTCTGTGTGGTCAGGTTATTGGTTGAATTCCCAGCAAC |
| | | AGGTGGTGTGATTCCATCTTGGCAATTTCAGCCTTTAAAGCTGA |
| | | TCCGATATGTCACAACTTTTGATTTCTTCCTGGCAGCCTGTGAG |
| | | ATTATCTTTTGTTTCTTTATCTTTTACTATGTGGTGGAAGAGAT |
| | | ATTGGAAATTCGCATTCACAAACTACACTATTTCAGGAGTTTCT |
| | | GGAATTGTCTGGATGTTGTGATCGTTGTGCTGTCAGTGGTAGCT |
| | | ATAGGAATTAACATATACAGAACATCAAATGTGGAGGTGCTACT |
| | | ACAGTTTCTGGAAGATCAAAATACTTTCCCCAACTTTGAGCATC |
| | | TGGCATATTGGCAGATACAGTTCAACAATATAGCTGCTGTCACA |
| | | GTATTTTTTGTCTGGATTAAGCTCTTCAAATTCATCAATTTTAA |
| | | CAGGACCATGAGCCAGCTCTCGACAACCATGTCTCGATGTGCCA |
| | | AAGACCTGTTTGGCTTTGCTATTATGTTCTTCATTATTTTCCTA |
| | | GCGTATGCTCAGTTGGCATACCTTGTCTTTGGCACTCAGGTCGA |
| | | TGACTTCAGTACTTTCCAAGAGTGTATCTTCACTCAATTCCGTA |
| | | TCATTTTGGGCGATATCAACTTTGCAGAGATTGAGGAAGCTAAT |
| | | CGAGTTTTGGGACCAATTTATTTCACTACATTTGTGTTCTTTAT |
| | | GTTCTTCATTCTTTTGAATATGTTTTTGGCTATCATCAATGATA |
| | | CTTACTCTGAAGTGAAATCTGACTTGGCACAGCAGAAAGCTGAA |
| | | ATGGAACTCTCAGATCTTATCAGAAAGGGCTACCATAAAGCTTT |
| | | GGTCAAACTAAAACTGAAAAAAAATACCGTGGATGACATTTCAG |
| | | AGAGTCTGCGGCAAGGAGGAGGCAAGTTAAACTTTGACGAACTT |
| | | CGACAAGATCTCAAAGGGAAGGGCCATACTGATGCAGAGATTGA |
| | | GGCAATATTCACAAAGTACGACCAAGATGGAGACCAAGAACTGA |
| | | CCGAACATGAACATCAGCAGATGAGAGACGACTTGGAGAAAGAG |
| | | AGGGAGGACCTGGATTTGGATCACAGTTCTTTACCACGTCCCAT |
| | | GAGCAGCCGAAGTTTCCCTCGAAGCCTGGATGACTCTGAGGAGG |
| | | ATGACGATGAAGATAGCGGACATAGCTCCAGAAGGAGGGGAAGC |
| | | ATTTCTAGTGGCGTTTCTTACGAAGAGTTTCAAGTCCTGGTGAG |
| | | ACGAGTGGACCGGATGGAGCATTCCATCGGCAGCATAGTGTCCA |
| | | AGATTGACGCCGTGATCGTGAAGCTAGAGATTATGGAGCGAGCC |
| | | AAACTGAAGAGGAGGGAGGTGCTGGGAAGGCTGTTGGATGGGGT |
| | | GGCCGAGGATGAAAGGCTGGGTCGTGACAGTGAAATCCATAGGG |
| | | AACAGATGGAACGGCTAGTACGTGAAGAGTTGGAACGCTGGGAA |
| | | TCCGATGATGCAGCTTCCCAGATCAGTCATGGTTTAGGCACGCC |
| | | AGTGGGACTAAATGGTCAACCTCGCCCCAGAAGCTCCCGCCCAT |
| | | CTTCCTCCCAATCTACAGAAGGCATGGAAGGTGCAGGTGGAAAT |
| | | GGGAGTTCTAATGTCCACGTATGATATGTGTGTTTCAGTATGTG |
| | | TGTTTCTAATAAGTGAGGAAGTGGCTGTCCTGAATTGCTGTAAC |
| | | AAGCACACTATTTATATGCCCTGACCACCATAGGATGCTAGTCT |
| | | TTGTGACCGATTGCTAATCTTCTGCACTTTAATTTATTTTATAT |
| | | AAACTTTACCCATGGTTCAAAGATTTTTTTTTCTTTTTCTCATA |
| | | TAAGAAATCTAGGTGTAAATATTGAGTACAGAAAAAAAATCTTC |
| | | ATGATGTGTATTGAGCGGTACGCCCAGTTGCCACCATGACTGAG |
| | | TCTTCTCAGTTGACAATGAAGTAGCCTTTTAAAGCTAGAAAACT |
| | | GTCAAAGGGCTTCTGAGTTTCATTTCCAGTCACAAAAATCAGTA |
| | | TTGTTATTTTTTTCCAAGAGTGTGAAGGAAAATGGGGCATTCCT |
| | | TTCCACTCTGGCATAGTTCATGAGCTTAATACATAGCTTTCTTT |
| | | TAAGAAAGGAGCCTTTTTTTTCAACTAGCTTCCTGGGGTAAACT |
| | | TTTCTAAAAGATAAAATGGAAAGGAACTCCAAACTATGATAGAA |
| | | TCTGTGTGAATGGTTAAGATGAATGTTAAATACTATGCTTTTTT |
| | | GTAAGTTGATCGTATCTGATGTCTGTGGGACTAACTGTATCACT |
| | | TAATTTTTACCTTATTTTGGCTCTAATTTGAATAAGCTGAGTAA |
| | | AACCACCAAAGATCAGTTATAGGATAAAATGGCATCTCTAACCA |
| | | TAACACAGGAGAATTGGAAGGAGCCCTAAGTTGTCACTCAGTTT |
| | | AATTTCTTTTAATGGTTAGTTTAGCCTAAAGATTTATCTGCATA |
| | | TTCTTTTTCCCATGTGGCTCTACTCATTTGCAACTGAATTTAAT |
| | | GTTATAACTCATCTAGTGAGACCAACTTACTAAATTTTTAGTAT |
| | | GCACTGAAAGTTTTTATCCAACAATTATGTTCATTTTAAGCAAA |
| | | ATTTTAAGAAAGTTTTGAAATTCATAAAGCATTTGGTTTTAAAC |
| | | TATTTTAAGAATATAGTACTCGGTCAGGTATGACGGCTCACGCC |
| | | TGTAATCCCAGCACTTTGGGAGGCCGAAACAGGCGAATCACTTG |
| | | AGCCCAGGAGTTCAAGACCAACATGGGCAATGTGGCGAAACTCC |
| | | ATCTCTACAAAAAATGCAAAAATAAAAAATATAGTACTCAAGTA |
| | | TTCTTGATCCTGTGTTTCAAAACTAGAATTTGTAATGCAAATGG |
| | | AGCTCAGTCTAATAAAAAAGAGGTTTTGGTATTAAAAGTTCATA |
| | | CATTAGACAGTATCAGCCAAAATTTGAGTTAGCAACACTGTTTT |
| | | CTTTACGAGAGGGTCTCACCCAAATTTATGGGGAGAAATCTATT |
| | | TCTCAAAAAAAAAAATCTTCTTTTACAGAAATGTTGAGTAAGGT |
| | | GACATTTTGAGCGCTAATAAGCAAAAGAGCATGCAGTGCTGTTG |
| | | AATAACCCTCACTTGGAGAACCAAGAGAATCCTGTCGTTTAATG |
| | | CTATATTTTAATTTCACAAGTTGTTCATTTAACTGGTAGAATGT |
| | | CAGTCCAATCTCCAATGAGAACATGAGCAAATAGACCTTTCCAG |
| | | GTTGAAAGTGAAACATACTGGGTTTCTGTAAGTTTTTCCTCATG |
| | | GCTTCATCTCTATCTTTACTTTCTCTTGAATATGCTACACAAAG |
| | | TTCTTTATTACTACATACTAAAGTTTGCATTCCAGGGATATTGA |
| | | CTGTACATATTTATGTATATGTACCATGTTGTTACATGTAAACA |
| | | AACTTCAATTTGAAGTGCAGCTATTATGTGGTATCCATGTGTAT |
| | | CGACCATGTGCCATATATCAATTATGGTCACTAGAAAGTCTCTT |
| | | TATGATACTTTTTATTGTACTGTTTTTCATTTCACTTGCAAAAT |
| | | TTTGCAGAATTCCTCCTTTCTACCCATAAATTACATATATTTTT |
| | | CTTCTTTAGTCATGGAGAACTCCCCCCCTCATCTCTTCCCTATT |
| | | ATCTTTCCCTGTGTACTGGTATTATTAAAAAGACATTACATACG |
| | | CAAGTCTTTCTCGACAATCAAGAATGTTATTAATGTGTAATACT |
| | | GAGCACTTTACTTCTTAATAAAAACTTGATATAGTAGCA |
| | | (SEQ ID NO: 244) |
| | transcript | ATGTCTTCTCTCTCTTACAGCTCTTCAAATTCATCAATTTTAAC |
| | ENST0000 | AGGACCATGAGCCAGCTCTCGACAACCATGTCTCGATGTGCCAA |
| | 0502363.1 | AGACCTGTTTGGCTTTGCTATTATGTTCTTCATTATTTTCCTAG |
| | | CGTATGCTCAGTTGGCATACCTTGTCTTTGGCACTCAGGTCGAT |
| | | GACTTCAGTACTTTCCAAGAGTGTATCTTCACTCAATTCCGTAT |
| | | CATTTTGGGCGATATCAACTTTGCAGAGATTGAGGAAGCTAATC |
| | | GAGTTTTGGGACCAATTTATTTCACTACATTTGTGTTCTTTATG |
| | | TTCTTCATTCTTTTGAATATGTTTTTGGCTATCATCAATGATAC |
| | | TTACTCTGAAGTGAAATCTGACTTGGCACAGCAGAAAGCTGAAA |
| | | TGGAACTCTCAGATCTTATCAGAAAGGGCTACCATAAAGCTTTG |
| | | GTCAAACTAAAACTGAAAAAAAATACCGTGGATGACATTTCAGA |
| | | GAGTCTGCGGCAAGGAGGAGGCAAGTTAAACTTTGACGAACTTC |
| | | GACAAGATCTCAAAGGGAAGGGCCATACTGATGCAGAGATTGAG |
| | | GCAATATTCACAAAGTACGACCAAGATGGAGACCAAGAACTGAC |
| | | CGAACATGAACATCAGCAGATGAGAGACGACTTGGAGAAAGAGA |
| | | GGGAGGACCTGGATTTGGATCACAGTTCTTTACCACGTCCCATG |
| | | AGCAGCCGAAGTTTCCCTCGAAGCCTGGATGACTCTGAGGAGGA |
| | | TGACGATGAAGATAGCGGACATAGCTCCAGAAGGAGGGGAAGCA |
| | | TTTCTAGTGGCGTTTCTTACGAAGAGTTTCAAGTCCTGGTGAGA |
| | | CGAGTGGACCGGATGGAGCATTCCATCGGCAGCATAGTGTCCAA |
| | | GATTGACGCCGTGATCGTGAAGCTAGAGATTATGGAGCGAGCCA |
| | | AACTGAAGAGGAGGGAGGTGCTGGGAAGGCTGTTGGATGGGGTG |
| | | GCCGAGGATGAAAGGCTGGGTCGTGACAGTGAAATCCATAGGGA |
| | | ACAGATGGAACGGCTAGTACGTGAAGAGTTGGAACGCTGGGAAT |
| | | CCGATGATGCAGCTTCCCAGATCAGTCATGGTTTAGGCACGCCA |
| | | GTGGGACTAAATGGTCAACCTCGCCCCAGAAGCTCCCGCCCATC |
| | | TTCCTCCCAATCTACAGAAGGCATGGAAGGTGCAGGTGGAAATG |
| | | GGAGTTCTAATGTCCACGTATGATATGTGTGTTT |
| | | (SEQ ID NO: 245) |
| | transcript | GAATGATAGGGGAAAGGAAGGCAAGGGTGAGAGAAGACCTTGTG |
| | ENST0000 | TGAATTTGTCCAAAATGTTTATCCACAGGAACAATCCCTTTGTG |
| | 0506367.1 | AAGGCTGCTGGTATGTGAATGTGTGCCGGTTCCCTTGGGGCGTT |
| | | CATTTGGATCTTTCTGTGTTCCAGTGACCTACGGCATGATGAGC |
| | | TCCAATGTGTACTACTACACCCGGATGATGTCACAGCTCTTCCT |
| | | AGACACCCCCGTGTCCAAAACGGAGAAAACTAACTTTAAAACTC |
| | | TGTCTTCCATGGAAGACTTCTGGAAGTTCACAGAAGGCTCCTTA |
| | | TTGGATGGGCTGTACTGGAAGATGCAGCCCAGCAACCAGACTGA |
| | | AGCTGACAACCGAAGTTTCATCTTCTATGAGAACCTGCTGTTAG |
| | | GGGTTCCACGAATACGGCAACTCCGAGTCAGAAATGGATCCTGC |
| | | TCTATCCCCCAGGACTTGAGAGATGAAATTAAAGAGTGCTATGA |
| | | TGTCTACTCTGTCAGTAGTGAAGATAGGGCTCCCTTTGGGCCCC |
| | | GAAATGGAACCGCTTGGATCTACACAAGTGAAAAAGACTTGAAT |
| | | GGTAGTAGCCACTGGGGAATCATTGCAACTTATAGTGGAGCTGG |
| | | CTATTATCTGGATTTGTCAAGAACAAGAGAGGAAACAGCTGCAC |
| | | AAGTTGCTAGCCTCAAGAAAAATGTCTGGCTGG |
| | | (SEQ ID NO: 246) |
| | transcript | CCAGTCGGCGGCGGGGACCCGCTGCATCGCCACCTCCCCCTGGA |
| | ENST0000 | AGGGCAGCCGCCCCGAGTGGCCTGGGCGGAGAGGCTGGTTCGCG |
| | 0506727.1 | GGCTGCGAGGTCTCTGGGGAACAAGACTCATGGAGGAAAGCAGC |
| | | ACTAACCGAGAGAAATACCTTAAAAGTGTTTTACGGGAACTGGT |
| | | CACATACCTCCTTTTTCTCATAGTCTTGTGCATCTGAACAATCC |
| | | CTTTGTGAAGGCTGCTGGTATGTGAATGTGTGCCGGTTCCCTTG |
| | | GGGCGTTCATTTGGATCTTTCTGTGTTCCAGTGACCTACGGCAT |
| | | GATGAGCTCCAATGTGTACTACTACACCCGGATGATGTCACAGC |
| | | TCTTCCTAGACACCCCCGTGTCCAAAACGGAGAAAACTAACTTT |
| | | AAAACTCTGTCTTCCATGGAAGACTTCTGGAAGTTCACAGAAGG |
| | | CTCCTTATTGGATGGGCTGTACTGGAAGATGCAGCCCAGCAACC |
| | | AGACTGAAGCTGACAACCGAAGTTTCATCTTCTATGAGAACCTG |
| | | CTGTTAGGGGTTCCACGAATAC |
| | | (SEQ ID NO: 247) |
| | transcript | AATGGTAGTAGCCACTGGGGAATCATTGCAACTTATAGTGGAGC |
| | ENST0000 | TGGCTATTATCTGGATTTGTCAAGAACAAGAGAGGAAACAGCTG |
| | 0508588.5 | CACAAGTTGCTAGCCTCAAGAAAAATGTCTGGCTGGACCGAGGA |
| | | ACCAGGGCAACTTTTATTGACTTCTCAGTGTACAACGCCAACAT |
| | | TAACCTGTTCTGTGTGGTCAGCTGTCAGTGGTAGCTATAGGAAT |
| | | TAACATATACAGAACATCAAATGTGGAGGTGCTACTACAGTTTC |
| | | TGGAAGATCAAAATACTTTCCCCAACTTTGAGCATCTGGCATAT |
| | | TGGCAGATACAGTTCAACAATATAGCTGCTGTCACAGTATTTTT |
| | | TGTCTGGATTAAGCTCTTCAAATTCATCAATTTTAACAGGACCA |
| | | TGAGCCAGCTCTCGACAACCATGTCTCGATGTGCCAAAGACCTG |
| | | TTTGGCTTTGCTATTATGTTCTTCATTATTTTCCTAGCGTATGC |
| | | TCAGTTGGCATACCTTGTCTTTGGCACTCAGGTCGATGACTTCA |
| | | GTACTTTCCAAGAGTGTATCTTCACTCAATTCCGTATCATTTTG |
| | | GGCGATATCAACTTTGCAGAGATTGAGGAAGCTAATCGAGTTTT |
| | | GGGACCAATTTATTTCACTACATTTGTGTTCTTTATGTTCTTCA |
| | | TTCTTTTGAATATGTTTTTGGCTATCATCAATGATACTTACTCT |
| | | GAAGTGAAATCTGACTTGGCACAGCAGAAAGCTGAAATGGAACT |
| | | CTCAGATCTTATCAGAAAGGGCTACCATAAAGCTTTGGTCAAAC |
| | | TAAAACTGAAAAAAAATACCGTGGATGACATTTCAGAGAGTCTG |
| | | CGGCAAGGAGGAGGCAAGTTAAACTTTGACGAACTTCGACAAGA |
| | | TCTCAAAGGGAAGGGCCATACTGATGCAGAGATTGAGGCAATAT |
| | | TCACAAAGTACGACCAAGATGGAGACCAAGAACTGACCGAACAT |
| | | GAACATCAGCAGATGAGAGACGACTTGGAGAAAGAGAGGGAGGA |
| | | CCTGGATTTGGATCACAGTTCTTTACCACGTCCCATGAGCAGCC |
| | | GAAGTTTCCCTCGAAGCCTGGATGACTCTGAGGAGGATGACGAT |
| | | GAAGATAGCGGACATAGCTCCAGAAGGAGGGGAAGCATTTCTAG |
| | | TGGCGTTTCTTACGAAGAGTTTCAAGTCCTGGTGAGACGAGTGG |
| | | ACCGGATGGAGCATTCCATCGGCAGCATAGTGTCCAAGATTGAC |
| | | GCCGTGATCGTGAAGCTAGAGATTATGGAGCGAGCCAAACTGAA |
| | | GAGGAGGGAGGTGCTGGGAAGGCTGTTGGATGGGGTGGCCGAGG |
| | | ATGAAAGGCTGGGTCGTGACAGTGAAATCCATAGGGAACAGATG |
| | | GAACGGCTAGTACGTGAAGAGTTGGAACGCTGGGAATCCGATGA |
| | | TGCAGCTTCCCAGATCAGTCATGGTTTAGGCACGCCAGTGGGAC |
| | | TAAATGGTCAACCTCGCCCCAGAAGCTCCCGCCCATCTTCCTCC |
| | | CAATCTACAGAAGGCATGGAAGGTGCAGGTGGAAATGGGAGTTC |
| | | TAATGTCCACGTATGATATGTGTGTTTCAGTATGTGTGTTTCTA |
| | | ATAAGTGAGGAAGTGGCTGTCCTGAATTGCTGTAACAAGCACAC |
| | | TATTTATATGCCCTGACCACCATAGGATGCTAGTCTTTGTGACC |
| | | GATTGCTAATCTTCTGCACTTTAATTTATTTTATATAAACTTTA |
| | | CCCA (SEQ ID NO: 248) |
| | transcript | ATACTTTCCCCAACTTTGAGCATCTGGCATATTGGCAGATACAG |
| | ENST0000 | TTCAACAATATAGCTGCTGTCACAGTATTTTTTGTCTGGATTAA |
| | 0511337.5 | GCTCTTCAAATTCATCAATTTTAACAGGACCATGAGCCAGCTCT |
| | | CGACAACCATGTCTCGATGTGCCAAAGACCTGTTTGGCTTTGCT |
| | | ATTATGTTCTTCATTATTTTCCTAGCGTATGCTCAGTTGGCATA |
| | | CCTTGTCTTTGGCACTCAGGTCGATGACTTCAGTACTTTCCAAG |
| | | AGTGTATAATATGTTTTTGGCTATCATCAATGATACTTACTCTG |
| | | AAGTGAAATCTGACTTGGCACAGCAGAAAGCTGAAATGGAACTC |
| | | TCAGATCTTATCAGAAAGGGCTACCATAAAGCTTTGGTCAAACT |
| | | AAAACTGAAAAAAAATACCGTGGATGACATTTCAGAGAGTCTGC |
| | | GGCAAGGAGGAGGCAAGTTAAACTTTGACGAACTTCGACAAGAT |
| | | CTCAAAGGGAAGGGCCATACTGATGCAGAGATTGAGGCAATATT |
| | | CACAAAGTACGACCAAGATGGAGACCAAGAACTGACCGAACATG |
| | | AACATCAGCAGATGAGAGACGACTTGGAGAAAGAGAGGGAGGAC |
| | | CTGGATTTGGATCACAGTTCTTTACCACGTCCCATGAGCAGCCG |
| | | AAGTTTCCCTCGAAGCCTGGATGACTCTGAGGAGGATGACGATG |
| | | AAGATAGCGGACATAGCTCCAGAAGGAGGGGAAGCATTTCTAGT |
| | | GGCGTTTCTTACGAAGAGTTTCAAGTCCTGGTGAGACGAGTGGA |
| | | CCGGATGGAGCATTCCATCGGCAGCATAGTGTCCAAGATTGACG |
| | | CCGTGATCGTGAAGCTAGAGATTATGGAGCGAGCCAAACTGAAG |
| | | AGGAGGGAGGTGCTGGGAAGGCTGTTGGATGGGGTGGCCGAGGA |
| | | TGAAAGGCTGGGTCGTGACAGTGAAATCCATAGGGAACAGATGG |
| | | AACGGCTAGTACGTGAAGAGTTGGAACGCTGGGAATCCGATGAT |
| | | GCAGCTTCCCAGATCAGTCATGGTTTAGGCACGCCAGTGGGACT |
| | | AAATGGTCAACCTCGCCCCAGAAGCTCCCGCCCATCTTCCTCCC |
| | | AATCTACAGAAGGCATGGAAGGTGCAGGTGGAAATGGGAGTTCT |
| | | AATGTCCACGTATGATATGTGTGTTTCAGTATGTGTGTTTCTAA |
| | | TAAGTGAGGAAGTGGCTGTCCTGAATTGCTGTAACAAGCACACT |
| | | ATTTATATGCCCTGACCACCATAGGATGCTAGTCTTTGTGACCG |
| | | ATTGCTAATCTTCTGCACTTTAATTTATTTTATATAAACTTTAC |
| | | CCATGGTTCAAAGATTTTTTTTTCTTTTTCTCATATAAGAAA |
| | | (SEQ ID NO: 249) |
| | transcript | ACAGTTCAACAATATAGCTGCTGTCACAGTATTTTTTGTCTGGAT |
| | ENST0000 | TAAGCTCTTCAAATTCATCAATTTTAACAGGACCATGAGCCAGCT |
| | 0512858.1 | CTCGACAACCATGTCTCGATGTGCCAAAGACCTGTTTGGCTTTGC |
| | | TATTATGTTCTTCATTATTTTCCTAGCGTATGCTCAGTTGGCATA |
| | | CCTTGTCTTTGGCACTCAGGTCGATGACTTCAGTACTTTCCAAGA |
| | | GTGTATAATATGTTTTTGGCTATCATCAATGATACTTACTCTGAA |
| | | GTGAAATCTGACTTGGCACAGCAGAAAGCTGAAATGGAACTCTCA |
| | | GATCTTATCAGAAAGGGCTACCATAAAGCTTTGGTCAAACTAAAA |
| | | CTGAAAAAAAATACCGTGGATGACATTTCAGAGAGTCTGCGGCAA |
| | | GGAGGAGGCAAGTTAAACTTTGACGAACTTCGACAAGATCTCAAA |
| | | GGGTGAGAATCATGCTTCCTGAGGTTCTGAAAAATTCCTGCTTCT |
| | | AAAGATAAATTCCTGGTGATAAGAGTATTTCTAGCCCAAGGGCTC |
| | | ATACAGATACTTTTTTTTTTTTTTTCCAGAGGCAGGTATCTTTCT |
| | | GGAACATGTTATAAGAGGAAAACTTGCCCCCATTTGGTGATTTCT |
| | | CCTTTCCTCCTGCATTTTGATGTCTCTGTGTTGAGGGTGAACTGG |
| | | GTACAAGGAATGATTTTTATCTGTATCCTCTCTCTAATTTCAGGA |
| | | AGGGCCATACTGATGCAGAGATTGAGGCAATATTCACAAAGTACG |
| | | ACCAAGATGGAGACCAAGAACTGACCGAACATGAACATCAGCAGA |
| | | TGAGAGACGACTTGGAGAAAGAGAGGGAGGACCTGGATTTGGATC |
| | | ACAGTTCTTTACCACGTCCCATGAGCAGCCGAAGTTTCCCTCGAA |
| | | GCCTGGATGACTCTGAGGAGGATGACGATGAAGATAGCGGACATA |
| | | GCTCCAGAAGGAGGGGAAGCATTTCTAGTGGCGTTTCTTACGAAG |
| | | AGTTTCAAGTCCTGGTGAGACGAGTGGACCGGATGGAGCATTCCA |
| | | TCGGCAGCATAGTGTCCAAGATTGACGCCGTGATCGTGAAGCTAG |
| | | AGATTATGGAGCGAGCCAAACTGAAGAGGAGGGAGGTGCTGGGAA |
| | | GGCTGTTGGATGGGGTGGCCGAGGATGAAAGGCTGGGTCGTGACA |
| | | GTGAAATCCATAGGGAACAGATGGAACGGCTAGTACGTGAAGAGT |
| | | TGGAACGCTGGGAATCCGATGATGCAGCTTCCCAGATCAGTCATG |
| | | GTTTAGGCACGCCAGTGGGACTAAATGGTCAACCTCGCCCCAGAA |
| | | GCTCCCGCCCATCTTCCTCCCAATCTACAGAAGGCATGGAAGGTG |
| | | CAGGTGGAAATGGGAGTTCTAATGTCCACGTATGATATGTGTGTT |
| | | TCAGTATGTGTGTTTCTAATAAGTGAGGAAGTGGCTGTCCTGAAT |
| | | TGCTGTAACAAGCACACTATTTATATGCCCTGACCACCATAGGAT |
| | | GCTAGTCTTTGTGACCGATTGCTAATCTTCTGCACTTTAATTTAT |
| | | TTTATATAAACTTTACCCATGGTT |
| | | (SEQ ID NO: 250) |

Translation modulation mechanism

| | |
|---|---|
| PKD2 5′ UTR sequence | AGGCGGCGGCGGGCGCCGGGAAGAAAGGAACATGGCTCCTGAGGC |
| (ENST00000237596.7 | GCACAGCGCCGAGCGCGGCGCCGCGCACCCGCGCGCCGGACGCCA |
| | GTGACCGCG (SEQ ID NO: 251) |

TABLE 4
PKD2 ASO sequences

| SEQ | | | | Oligo Start (chr | Oligo End (chr |
|---|---|---|---|---|---|
| ID | Event | Sequence | Chr | coordinate) | coordinate) |
| 1 | Alt 5′ss | GTACTTTCAAAGTTATTT | chr4 | 88036361 | 88036379 |
| 2 | Alt 5′ss | GAGAGGTACTTTCAAAGT | chr4 | 88036366 | 88036384 |
| 3 | Alt 5′ss | TGATAGAGAGGTACTTTC | chr4 | 88036371 | 88036389 |
| 4 | Alt 5′ss | TTCTGTGATAGAGAGGTA | chr4 | 88036376 | 88036394 |
| 5 | Alt 5′ss | CAATTTTCTGTGATAGAG | chr4 | 88036381 | 88036399 |
| 6 | Alt 5′ss | ATGAACAATTTTCTGTGA | chr4 | 88036386 | 88036404 |
| 7 | Alt 5′ss | GCCAAATGAACAATTTTC | chr4 | 88036391 | 88036409 |
| 8 | Alt 5′ss | ATGAAGCCAAATGAACAA | chr4 | 88036396 | 88036414 |
| 9 | Alt 5′ss | AAATGATGAAGCCAAATG | chr4 | 88036401 | 88036419 |
| 10 | Alt 5′ss | CATTGAAATGATGAAGCC | chr4 | 88036406 | 88036424 |
| 11 | Alt 5′ss | TCATGCATTGAAATGATG | chr4 | 88036411 | 88036429 |
| 12 | Alt 5′ss | GATACTCATGCATTGAAA | chr4 | 88036416 | 88036434 |
| 13 | Alt 5′ss | CTGTCGATACTCATGCAT | chr4 | 88036421 | 88036439 |
| 14 | Alt 5′ss | AGGTCCTGTCGATACTCA | chr4 | 88036426 | 88036444 |
| 15 | Alt 5′ss | AAAGCAGGTCCTGTCGAT | chr4 | 88036431 | 88036449 |
| 16 | Alt 5′ss | AATGCAAAGCAGGTCCTG | chr4 | 88036436 | 88036454 |
| 17 | Alt 5′ss | TGTTAAATGCAAAGCAGG | chr4 | 88036441 | 88036459 |
| 18 | Alt 5′ss | CACAGTGTTAAATGCAAA | chr4 | 88036446 | 88036464 |
| 19 | Alt 5′ss | TCTCACACAGTGTTAAAT | chr4 | 88036451 | 88036469 |
| 20 | Alt 5′ss | TTACGTCTCACACAGTGT | chr4 | 88036456 | 88036474 |
| 21 | Alt 5′ss | ATAACTTACGTCTCACAC | chr4 | 88036461 | 88036479 |
| 22 | Alt 5′ss | TCACCATAACTTACGTCT | chr4 | 88036466 | 88036484 |
| 23 | Alt 5′ss | ACAACTCACCATAACTTA | chr4 | 88036471 | 88036489 |
| 24 | Alt 5′ss | TTCTAACAACTCACCATA | chr4 | 88036476 | 88036494 |
| 25 | Alt 5′ss | GTAACTTCTAACAACTCA | chr4 | 88036481 | 88036499 |
| 26 | Alt 5′ss | GAACAGTAACTTCTAACA | chr4 | 88036486 | 88036504 |
| 27 | Alt 5′ss | AGTAGGAACAGTAACTTC | chr4 | 88036491 | 88036509 |
| 28 | Alt 5′ss | TTGAGAGTAGGAACAGTA | chr4 | 88036496 | 88036514 |
| 29 | Alt 5′ss | ACCCCCTTTGAGAGTAGG | chr4 | 88036503 | 88036521 |
| 30 | Alt 5′ss | AGTTTACCCCCTTTGAGA | chr4 | 88036508 | 88036526 |
| 31 | Alt 5′ss | ATGTTAGTTTACCCCCTT | chr4 | 88036513 | 88036531 |
| 32 | Alt 5′ss | TCTCAATGTTAGTTTACC | chr4 | 88036518 | 88036536 |
| 33 | Alt 5′ss | AAAGTTCTCAATGTTAGT | chr4 | 88036523 | 88036541 |
| 34 | Alt 5′ss | CAGGCAAAGTTCTCAATG | chr4 | 88036528 | 88036546 |
| 35 | Alt 5′ss | AGGCACAGGCAAAGTTCT | chr4 | 88036533 | 88036551 |
| 36 | Alt 5′ss | GTGCAAGGCACAGGCAAA | chr4 | 88036538 | 88036556 |
| 37 | Alt 5′ss | GCACAGTGCAAGGCACAG | chr4 | 88036543 | 88036561 |
| 38 | Alt 5′ss | ACTCAGCACAGTGCAAGG | chr4 | 88036548 | 88036566 |
| 39 | Alt 5′ss | GAAACACTCAGCACAGTG | chr4 | 88036553 | 88036571 |
| 40 | Alt 5′ss | GATATGAAACACTCAGCA | chr4 | 88036558 | 88036576 |
| 41 | Alt 5′ss | GGTAAGATATGAAACACT | chr4 | 88036563 | 88036581 |
| 42 | Alt 5′ss | AATAAGGTAAGATATGAA | chr4 | 88036568 | 88036586 |
| 43 | Alt 5′ss | AATTAAATAAGGTAAGAT | chr4 | 88036573 | 88036591 |
| 44 | Alt 5′ss | ATAGAAATTAAATAAGGT | chr4 | 88036578 | 88036596 |
| 45 | Alt 5′ss | AGACTATAGAAATTAAAT | chr4 | 88036583 | 88036601 |
| 46 | Alt 5′ss | GAGTTAGACTATAGAAAT | chr4 | 88036588 | 88036606 |
| 47 | Alt 5′ss | TTATAGAGTTAGACTATA | chr4 | 88036593 | 88036611 |
| 48 | Alt 5′ss | TTACCTTATAGAGTTAGA | chr4 | 88036598 | 88036616 |
| 49 | Alt 5′ss | AGTACTTACCTTATAGAG | chr4 | 88036603 | 88036621 |
| 50 | NMD exon | ATAAATCGAACCATCGTA | chr4 | 88030981 | 88030999 |
| 51 | NMD exon | AAATCATAAATCGAACCA | chr4 | 88030986 | 88031004 |
| 52 | NMD exon | TTGAAAAATCATAAATCG | chr4 | 88030991 | 88031009 |
| 53 | NMD exon | TGAAGTTGAAAAATCATA | chr4 | 88030996 | 88031014 |
| 54 | NMD exon | CACCATGAAGTTGAAAAA | chr4 | 88031001 | 88031019 |
| 55 | NMD exon | CACATCACCATGAAGTTG | chr4 | 88031006 | 88031024 |
| 56 | NMD exon | ACTTTCACATCACCATGA | chr4 | 88031011 | 88031029 |
| 57 | NMD exon | GTATCACTTTCACATCAC | chr4 | 88031016 | 88031034 |
| 58 | NMD exon | AATGTGTATCACTTTCAC | chr4 | 88031021 | 88031039 |
| 59 | NMD exon | TATAGAATGTGTATCACT | chr4 | 88031026 | 88031044 |
| 60 | NMD exon | GTTTCTATAGAATGTGTA | chr4 | 88031031 | 88031049 |
| 61 | NMD exon | GTGTGGTTTCTATAGAAT | chr4 | 88031036 | 88031054 |
| 62 | NMD exon | TGAAAGTGTGGTTTCTAT | chr4 | 88031041 | 88031059 |
| 63 | NMD exon | AAAATTGAAAGTGTGGTT | chr4 | 88031046 | 88031064 |
| 64 | NMD exon | AATTCAAAATTGAAAGTG | chr4 | 88031051 | 88031069 |
| 65 | NMD exon | ACCAAAATTCAAAATTGA | chr4 | 88031056 | 88031074 |
| 66 | NMD exon | AAAAGACCAAAATTCAAA | chr4 | 88031061 | 88031079 |
| 67 | NMD exon | CTGGAAAAAGACCAAAAT | chr4 | 88031066 | 88031084 |
| 68 | NMD exon | GTAGCCTGGAAAAAGACC | chr4 | 88031071 | 88031089 |
| 69 | NMD exon | GTATGGTAGCCTGGAAAA | chr4 | 88031076 | 88031094 |
| 70 | NMD exon | TTAGAGTATGGTAGCCTG | chr4 | 88031081 | 88031099 |
| 71 | NMD exon | TCTTTAGAGTATGGTAGC | chr4 | 88031084 | 88031102 |
| 72 | NMD exon | CTCTATCTTTAGAGTATG | chr4 | 88031089 | 88031107 |
| 73 | NMD exon | TGTGGCTCTATCTTTAGA | chr4 | 88031094 | 88031112 |
| 74 | NMD exon | GGATCTGTGGCTCTATCT | chr4 | 88031099 | 88031117 |
| 75 | NMD exon | GACTGGGATCTGTGGCTC | chr4 | 88031104 | 88031122 |
| 76 | NMD exon | ACATGGCTGACTGGGATC | chr4 | 88031112 | 88031130 |
| 77 | NMD exon | TAATCACATGGCTGACTG | chr4 | 88031117 | 88031135 |
| 78 | NMD exon | CCTCATAATCACATGGCT | chr4 | 88031122 | 88031140 |
| 79 | NMD exon | TACCCTCATAATCACATG | chr4 | 88031125 | 88031143 |
| 80 | NMD exon | TCGCTTACCCTCATAATC | chr4 | 88031130 | 88031148 |
| 81 | NMD exon | ATTGGTCGCTTACCCTCA | chr4 | 88031135 | 88031153 |
| 82 | NMD exon | AGAGTATTGGTCGCTTAC | chr4 | 88031140 | 88031158 |
| 83 | NMD exon | ACTGTAGAGTATTGGTCG | chr4 | 88031145 | 88031163 |
| 84 | NMD exon | AATACACTGTAGAGTATT | chr4 | 88031150 | 88031168 |
| 85 | NMD exon | AATACAATACACTGTAGA | chr4 | 88031155 | 88031173 |
| 86 | NMD exon | CTGGCAATACAATACACT | chr4 | 88031160 | 88031178 |
| 87 | NMD exon | ACCATCTGGCAATACAAT | chr4 | 88031165 | 88031183 |
| 88 | NMD exon | GCAAAACCATCTGGCAAT | chr4 | 88031170 | 88031188 |
| 89 | NMD exon | GTTGGGCAAAACCATCTG | chr4 | 88031175 | 88031193 |
| 90 | NMD exon | GGCTAGTTGGGCAAAACC | chr4 | 88031180 | 88031198 |
| 91 | NMD exon | CATTAGGCTAGTTGGGCA | chr4 | 88031185 | 88031203 |
| 92 | NMD exon | ACTTACATTAGGCTAGTT | chr4 | 88031190 | 88031208 |
| 93 | NMD exon | AGAATACTTACATTAGGC | chr4 | 88031195 | 88031213 |
| 94 | NMD exon | TGTTTAGAATACTTACAT | chr4 | 88031200 | 88031218 |
| 95 | NMD exon | AAACATGTTTAGAATACT | chr4 | 88031205 | 88031223 |
| 96 | NMD exon | ACCTTAAACATGTTTAGA | chr4 | 88031210 | 88031228 |
| 97 | NMD exon | GGCCTACCTTAAACATGT | chr4 | 88031215 | 88031233 |
| 98 | NMD exon | AGCCTGGCCTACCTTAAA | chr4 | 88031220 | 88031238 |
| 99 | NMD exon | AGCTTAGCCTGGCCTACC | chr4 | 88031225 | 88031243 |
| 100 | uORF | TTTCTTCCCGGCGCCCGC | chr4 | 88007642 | 88007660 |
| 101 | uORF | CCTTTCTTCCCGGCGCCC | chr4 | 88007644 | 88007662 |
| 102 | uORF | TTCCTTTCTTCCCGGCGC | chr4 | 88007646 | 88007664 |
| 103 | uORF | TGTTCCTTTCTTCCCGGC | chr4 | 88007648 | 88007666 |
| 104 | uORF | CATGTTCCTTTCTTCCCG | chr4 | 88007650 | 88007668 |
| 105 | uORF | GCCATGTTCCTTTCTTCC | chr4 | 88007652 | 88007670 |
| 106 | uORF | GAGCCATGTTCCTTTCTT | chr4 | 88007654 | 88007672 |
| 107 | uORF | AGGAGCCATGTTCCTTTC | chr4 | 88007656 | 88007674 |
| 108 | uORF | TCAGGAGCCATGTTCCTT | chr4 | 88007658 | 88007676 |
| 109 | uORF | CCTCAGGAGCCATGTTCC | chr4 | 88007660 | 88007678 |
| 110 | uORF | CGCCTCAGGAGCCATGTT | chr4 | 88007662 | 88007680 |
| 111 | uORF | TGCGCCTCAGGAGCCATG | chr4 | 88007664 | 88007682 |
| 112 | uORF | TGTGCGCCTCAGGAGCCA | chr4 | 88007666 | 88007684 |
| 113 | uORF | CTGTGCGCCTCAGGAGCC | chr4 | 88007667 | 88007685 |
| 114 | Alt 5′ss | GUACUUUCAAAGUUAUUU | chr4 | 88036361 | 88036379 |
| 115 | Alt 5′ss | GAGAGGUACUUUCAAAGU | chr4 | 88036366 | 88036384 |
| 116 | Alt 5′ss | UGAUAGAGAGGUACUUUC | chr4 | 88036371 | 88036389 |
| 117 | Alt 5′ss | UUCUGUGAUAGAGAGGUA | chr4 | 88036376 | 88036394 |
| 118 | Alt 5′ss | CAAUUUUCUGUGAUAGAG | chr4 | 88036381 | 88036399 |
| 119 | Alt 5′ss | AUGAACAAUUUUCUGUGA | chr4 | 88036386 | 88036404 |
| 120 | Alt 5′ss | GCCAAAUGAACAAUUUUC | chr4 | 88036391 | 88036409 |
| 121 | Alt 5′ss | AUGAAGCCAAAUGAACAA | chr4 | 88036396 | 88036414 |
| 122 | Alt 5′ss | AAAUGAUGAAGCCAAAUG | chr4 | 88036401 | 88036419 |
| 123 | Alt 5′ss | CAUUGAAAUGAUGAAGCC | chr4 | 88036406 | 88036424 |
| 124 | Alt 5′ss | UCAUGCAUUGAAAUGAUG | chr4 | 88036411 | 88036429 |
| 125 | Alt 5′ss | GAUACUCAUGCAUUGAAA | chr4 | 88036416 | 88036434 |
| 126 | Alt 5′ss | CUGUCGAUACUCAUGCAU | chr4 | 88036421 | 88036439 |
| 127 | Alt 5′ss | AGGUCCUGUCGAUACUCA | chr4 | 88036426 | 88036444 |
| 128 | Alt 5′ss | AAAGCAGGUCCUGUCGAU | chr4 | 88036431 | 88036449 |
| 129 | Alt 5′ss | AAUGCAAAGCAGGUCCUG | chr4 | 88036436 | 88036454 |
| 130 | Alt 5′ss | UGUUAAAUGCAAAGCAGG | chr4 | 88036441 | 88036459 |
| 131 | Alt 5′ss | CACAGUGUUAAAUGCAAA | chr4 | 88036446 | 88036464 |
| 132 | Alt 5′ss | UCUCACACAGUGUUAAAU | chr4 | 88036451 | 88036469 |
| 133 | Alt 5′ss | UUACGUCUCACACAGUGU | chr4 | 88036456 | 88036474 |
| 134 | Alt 5′ss | AUAACUUACGUCUCACAC | chr4 | 88036461 | 88036479 |
| 135 | Alt 5′ss | UCACCAUAACUUACGUCU | chr4 | 88036466 | 88036484 |
| 136 | Alt 5′ss | ACAACUCACCAUAACUUA | chr4 | 88036471 | 88036489 |
| 137 | Alt 5′ss | UUCUAACAACUCACCAUA | chr4 | 88036476 | 88036494 |
| 138 | Alt 5′ss | GUAACUUCUAACAACUCA | chr4 | 88036481 | 88036499 |
| 139 | Alt 5′ss | GAACAGUAACUUCUAACA | chr4 | 88036486 | 88036504 |
| 140 | Alt 5′ss | AGUAGGAACAGUAACUUC | chr4 | 88036491 | 88036509 |
| 141 | Alt 5′ss | UUGAGAGUAGGAACAGUA | chr4 | 88036496 | 88036514 |
| 142 | Alt 5′ss | ACCCCCUUUGAGAGUAGG | chr4 | 88036503 | 88036521 |
| 143 | Alt 5′ss | AGUUUACCCCCUUUGAGA | chr4 | 88036508 | 88036526 |
| 144 | Alt 5′ss | AUGUUAGUUUACCCCCUU | chr4 | 88036513 | 88036531 |
| 145 | Alt 5′ss | UCUCAAUGUUAGUUUACC | chr4 | 88036518 | 88036536 |
| 146 | Alt 5′ss | AAAGUUCUCAAUGUUAGU | chr4 | 88036523 | 88036541 |
| 147 | Alt 5′ss | CAGGCAAAGUUCUCAAUG | chr4 | 88036528 | 88036546 |
| 148 | Alt 5′ss | AGGCACAGGCAAAGUUCU | chr4 | 88036533 | 88036551 |
| 149 | Alt 5′ss | GUGCAAGGCACAGGCAAA | chr4 | 88036538 | 88036556 |
| 150 | Alt 5′ss | GCACAGUGCAAGGCACAG | chr4 | 88036543 | 88036561 |
| 151 | Alt 5′ss | ACUCAGCACAGUGCAAGG | chr4 | 88036548 | 88036566 |
| 152 | Alt 5′ss | GAAACACUCAGCACAGUG | chr4 | 88036553 | 88036571 |
| 153 | Alt 5′ss | GAUAUGAAACACUCAGCA | chr4 | 88036558 | 88036576 |
| 154 | Alt 5′ss | GGUAAGAUAUGAAACACU | chr4 | 88036563 | 88036581 |
| 155 | Alt 5′ss | AAUAAGGUAAGAUAUGAA | chr4 | 88036568 | 88036586 |
| 156 | Alt 5′ss | AAUUAAAUAAGGUAAGAU | chr4 | 88036573 | 88036591 |
| 157 | Alt 5′ss | AUAGAAAUUAAAUAAGGU | chr4 | 88036578 | 88036596 |
| 158 | Alt 5′ss | AGACUAUAGAAAUUAAAU | chr4 | 88036583 | 88036601 |
| 159 | Alt 5′ss | GAGUUAGACUAUAGAAAU | chr4 | 88036588 | 88036606 |
| 160 | Alt 5′ss | UUAUAGAGUUAGACUAUA | chr4 | 88036593 | 88036611 |
| 161 | Alt 5′ss | UUACCUUAUAGAGUUAGA | chr4 | 88036598 | 88036616 |
| 162 | Alt 5′ss | AGUACUUACCUUAUAGAG | chr4 | 88036603 | 88036621 |
| 163 | NMD exon | AUAAAUCGAACCAUCGUA | chr4 | 88030981 | 88030999 |
| 164 | NMD exon | AAAUCAUAAAUCGAACCA | chr4 | 88030986 | 88031004 |
| 165 | NMD exon | UUGAAAAAUCAUAAAUCG | chr4 | 88030991 | 88031009 |
| 166 | NMD exon | UGAAGUUGAAAAAUCAUA | chr4 | 88030996 | 88031014 |
| 167 | NMD exon | CACCAUGAAGUUGAAAAA | chr4 | 88031001 | 88031019 |
| 168 | NMD exon | CACAUCACCAUGAAGUUG | chr4 | 88031006 | 88031024 |
| 169 | NMD exon | ACUUUCACAUCACCAUGA | chr4 | 88031011 | 88031029 |
| 170 | NMD exon | GUAUCACUUUCACAUCAC | chr4 | 88031016 | 88031034 |
| 171 | NMD exon | AAUGUGUAUCACUUUCAC | chr4 | 88031021 | 88031039 |
| 172 | NMD exon | UAUAGAAUGUGUAUCACU | chr4 | 88031026 | 88031044 |
| 173 | NMD exon | GUUUCUAUAGAAUGUGUA | chr4 | 88031031 | 88031049 |
| 174 | NMD exon | GUGUGGUUUCUAUAGAAU | chr4 | 88031036 | 88031054 |
| 175 | NMD exon | UGAAAGUGUGGUUUCUAU | chr4 | 88031041 | 88031059 |
| 176 | NMD exon | AAAAUUGAAAGUGUGGUU | chr4 | 88031046 | 88031064 |
| 177 | NMD exon | AAUUCAAAAUUGAAAGUG | chr4 | 88031051 | 88031069 |
| 178 | NMD exon | ACCAAAAUUCAAAAUUGA | chr4 | 88031056 | 88031074 |
| 179 | NMD exon | AAAAGACCAAAAUUCAAA | chr4 | 88031061 | 88031079 |
| 180 | NMD exon | CUGGAAAAAGACCAAAAU | chr4 | 88031066 | 88031084 |
| 181 | NMD exon | GUAGCCUGGAAAAAGACC | chr4 | 88031071 | 88031089 |
| 182 | NMD exon | GUAUGGUAGCCUGGAAAA | chr4 | 88031076 | 88031094 |
| 183 | NMD exon | UUAGAGUAUGGUAGCCUG | chr4 | 88031081 | 88031099 |
| 184 | NMD exon | UCUUUAGAGUAUGGUAGC | chr4 | 88031084 | 88031102 |
| 185 | NMD exon | CUCUAUCUUUAGAGUAUG | chr4 | 88031089 | 88031107 |
| 186 | NMD exon | UGUGGCUCUAUCUUUAGA | chr4 | 88031094 | 88031112 |
| 187 | NMD exon | GGAUCUGUGGCUCUAUCU | chr4 | 88031099 | 88031117 |
| 188 | NMD exon | GACUGGGAUCUGUGGCUC | chr4 | 88031104 | 88031122 |
| 189 | NMD exon | ACAUGGCUGACUGGGAUC | chr4 | 88031112 | 88031130 |
| 190 | NMD exon | UAAUCACAUGGCUGACUG | chr4 | 88031117 | 88031135 |
| 191 | NMD exon | CCUCAUAAUCACAUGGCU | chr4 | 88031122 | 88031140 |
| 192 | NMD exon | UACCCUCAUAAUCACAUG | chr4 | 88031125 | 88031143 |
| 193 | NMD exon | UCGCUUACCCUCAUAAUC | chr4 | 88031130 | 88031148 |
| 194 | NMD exon | AUUGGUCGCUUACCCUCA | chr4 | 88031135 | 88031153 |
| 195 | NMD exon | AGAGUAUUGGUCGCUUAC | chr4 | 88031140 | 88031158 |
| 196 | NMD exon | ACUGUAGAGUAUUGGUCG | chr4 | 88031145 | 88031163 |
| 197 | NMD exon | AAUACACUGUAGAGUAUU | chr4 | 88031150 | 88031168 |
| 198 | NMD exon | AAUACAAUACACUGUAGA | chr4 | 88031155 | 88031173 |
| 199 | NMD exon | CUGGCAAUACAAUACACU | chr4 | 88031160 | 88031178 |
| 200 | NMD exon | ACCAUCUGGCAAUACAAU | chr4 | 88031165 | 88031183 |
| 201 | NMD exon | GCAAAACCAUCUGGCAAU | chr4 | 88031170 | 88031188 |
| 202 | NMD exon | GUUGGGCAAAACCAUCUG | chr4 | 88031175 | 88031193 |
| 203 | NMD exon | GGCUAGUUGGGCAAAACC | chr4 | 88031180 | 88031198 |
| 204 | NMD exon | CAUUAGGCUAGUUGGGCA | chr4 | 88031185 | 88031203 |
| 205 | NMD exon | ACUUACAUUAGGCUAGUU | chr4 | 88031190 | 88031208 |
| 206 | NMD exon | AGAAUACUUACAUUAGGC | chr4 | 88031195 | 88031213 |
| 207 | NMD exon | UGUUUAGAAUACUUACAU | chr4 | 88031200 | 88031218 |
| 208 | NMD exon | AAACAUGUUUAGAAUACU | chr4 | 88031205 | 88031223 |
| 209 | NMD exon | ACCUUAAACAUGUUUAGA | chr4 | 88031210 | 88031228 |
| 210 | NMD exon | GGCCUACCUUAAACAUGU | chr4 | 88031215 | 88031233 |
| 211 | NMD exon | AGCCUGGCCUACCUUAAA | chr4 | 88031220 | 88031238 |
| 212 | NMD exon | AGCUUAGCCUGGCCUACC | chr4 | 88031225 | 88031243 |
| 213 | uORF | UUUCUUCCCGGCGCCCGC | chr4 | 88007642 | 88007660 |
| 214 | uORF | CCUUUCUUCCCGGCGCCC | chr4 | 88007644 | 88007662 |
| 215 | uORF | UUCCUUUCUUCCCGGCGC | chr4 | 88007646 | 88007664 |
| 216 | uORF | UGUUCCUUUCUUCCCGGC | chr4 | 88007648 | 88007666 |
| 217 | uORF | CAUGUUCCUUUCUUCCCG | chr4 | 88007650 | 88007668 |
| 218 | uORF | GCCAUGUUCCUUUCUUCC | chr4 | 88007652 | 88007670 |
| 219 | uORF | GAGCCAUGUUCCUUUCUU | chr4 | 88007654 | 88007672 |
| 220 | uORF | AGGAGCCAUGUUCCUUUC | chr4 | 88007656 | 88007674 |
| 221 | uORF | UCAGGAGCCAUGUUCCUU | chr4 | 88007658 | 88007676 |
| 222 | uORF | CCUCAGGAGCCAUGUUCC | chr4 | 88007660 | 88007678 |
| 223 | uORF | CGCCUCAGGAGCCAUGUU | chr4 | 88007662 | 88007680 |
| 224 | uORF | UGCGCCUCAGGAGCCAUG | chr4 | 88007664 | 88007682 |
| 225 | uORF | UGUGCGCCUCAGGAGCCA | chr4 | 88007666 | 88007684 |
| 226 | uORF | CUGUGCGCCUCAGGAGCC | chr4 | 88007667 | 88007685 |

*alt 5 ss refers to an alternative 5′ splice site of an intron.

TABLE 5
Exemplary PKD2 Vectorized ASO Sequences

| SEQ | | | | Oligo Start (chr | Oligo End (chr |
|---|---|---|---|---|---|
| ID | Event | Sequence | Chr | coordinate) | coordinate) |
| 1 | Alt 5′ss | GTACTTTCAAAGTTATTT | chr4 | 88036361 | 88036379 |
| 2 | Alt 5′ss | GAGAGGTACTTTCAAAGT | chr4 | 88036366 | 88036384 |
| 3 | Alt 5′ss | TGATAGAGAGGTACTTTC | chr4 | 88036371 | 88036389 |
| 4 | Alt 5′ss | TTCTGTGATAGAGAGGTA | chr4 | 88036376 | 88036394 |
| 5 | Alt 5′ss | CAATTTTCTGTGATAGAG | chr4 | 88036381 | 88036399 |
| 6 | Alt 5′ss | ATGAACAATTTTCTGTGA | chr4 | 88036386 | 88036404 |
| 7 | Alt 5′ss | GCCAAATGAACAATTTTC | chr4 | 88036391 | 88036409 |
| 8 | Alt 5′ss | ATGAAGCCAAATGAACAA | chr4 | 88036396 | 88036414 |
| 9 | Alt 5′ss | AAATGATGAAGCCAAATG | chr4 | 88036401 | 88036419 |
| 10 | Alt 5′ss | CATTGAAATGATGAAGCC | chr4 | 88036406 | 88036424 |
| 11 | Alt 5′ss | TCATGCATTGAAATGATG | chr4 | 88036411 | 88036429 |
| 12 | Alt 5′ss | GATACTCATGCATTGAAA | chr4 | 88036416 | 88036434 |
| 13 | Alt 5′ss | CTGTCGATACTCATGCAT | chr4 | 88036421 | 88036439 |
| 14 | Alt 5′ss | AGGTCCTGTCGATACTCA | chr4 | 88036426 | 88036444 |
| 15 | Alt 5′ss | AAAGCAGGTCCTGTCGAT | chr4 | 88036431 | 88036449 |
| 16 | Alt 5′ss | AATGCAAAGCAGGTCCTG | chr4 | 88036436 | 88036454 |
| 17 | Alt 5′ss | TGTTAAATGCAAAGCAGG | chr4 | 88036441 | 88036459 |
| 18 | Alt 5′ss | CACAGTGTTAAATGCAAA | chr4 | 88036446 | 88036464 |
| 19 | Alt 5′ss | TCTCACACAGTGTTAAAT | chr4 | 88036451 | 88036469 |
| 20 | Alt 5′ss | TTACGTCTCACACAGTGT | chr4 | 88036456 | 88036474 |
| 21 | Alt 5′ss | ATAACTTACGTCTCACAC | chr4 | 88036461 | 88036479 |
| 22 | Alt 5′ss | TCACCATAACTTACGTCT | chr4 | 88036466 | 88036484 |
| 23 | Alt 5′ss | ACAACTCACCATAACTTA | chr4 | 88036471 | 88036489 |
| 24 | Alt 5′ss | TTCTAACAACTCACCATA | chr4 | 88036476 | 88036494 |
| 25 | Alt 5′ss | GTAACTTCTAACAACTCA | chr4 | 88036481 | 88036499 |
| 26 | Alt 5′ss | GAACAGTAACTTCTAACA | chr4 | 88036486 | 88036504 |
| 27 | Alt 5′ss | AGTAGGAACAGTAACTTC | chr4 | 88036491 | 88036509 |
| 28 | Alt 5′ss | TTGAGAGTAGGAACAGTA | chr4 | 88036496 | 88036514 |
| 29 | Alt 5′ss | ACCCCCTTTGAGAGTAGG | chr4 | 88036503 | 88036521 |
| 30 | Alt 5′ss | AGTTTACCCCCTTTGAGA | chr4 | 88036508 | 88036526 |
| 31 | Alt 5′ss | ATGTTAGTTTACCCCCTT | chr4 | 88036513 | 88036531 |
| 32 | Alt 5′ss | TCTCAATGTTAGTTTACC | chr4 | 88036518 | 88036536 |
| 33 | Alt 5′ss | AAAGTTCTCAATGTTAGT | chr4 | 88036523 | 88036541 |
| 34 | Alt 5′ss | CAGGCAAAGTTCTCAATG | chr4 | 88036528 | 88036546 |
| 35 | Alt 5′ss | AGGCACAGGCAAAGTTCT | chr4 | 88036533 | 88036551 |
| 36 | Alt 5′ss | GTGCAAGGCACAGGCAAA | chr4 | 88036538 | 88036556 |
| 37 | Alt 5′ss | GCACAGTGCAAGGCACAG | chr4 | 88036543 | 88036561 |
| 38 | Alt 5′ss | ACTCAGCACAGTGCAAGG | chr4 | 88036548 | 88036566 |
| 39 | Alt 5′ss | GAAACACTCAGCACAGTG | chr4 | 88036553 | 88036571 |
| 40 | Alt 5′ss | GATATGAAACACTCAGCA | chr4 | 88036558 | 88036576 |
| 41 | Alt 5′ss | GGTAAGATATGAAACACT | chr4 | 88036563 | 88036581 |
| 42 | Alt 5′ss | AATAAGGTAAGATATGAA | chr4 | 88036568 | 88036586 |
| 43 | Alt 5′ss | AATTAAATAAGGTAAGAT | chr4 | 88036573 | 88036591 |
| 44 | Alt 5′ss | ATAGAAATTAAATAAGGT | chr4 | 88036578 | 88036596 |
| 45 | Alt 5′ss | AGACTATAGAAATTAAAT | chr4 | 88036583 | 88036601 |
| 46 | Alt 5′ss | GAGTTAGACTATAGAAAT | chr4 | 88036588 | 88036606 |
| 47 | Alt 5′ss | TTATAGAGTTAGACTATA | chr4 | 88036593 | 88036611 |
| 48 | Alt 5′ss | TTACCTTATAGAGTTAGA | chr4 | 88036598 | 88036616 |
| 49 | Alt 5′ss | AGTACTTACCTTATAGAG | chr4 | 88036603 | 88036621 |
| 50 | NMD exon | ATAAATCGAACCATCGTA | chr4 | 88030981 | 88030999 |
| 51 | NMD exon | AAATCATAAATCGAACCA | chr4 | 88030986 | 88031004 |
| 52 | NMD exon | TTGAAAAATCATAAATCG | chr4 | 88030991 | 88031009 |
| 53 | NMD exon | TGAAGTTGAAAAATCATA | chr4 | 88030996 | 88031014 |
| 54 | NMD exon | CACCATGAAGTTGAAAAA | chr4 | 88031001 | 88031019 |
| 55 | NMD exon | CACATCACCATGAAGTTG | chr4 | 88031006 | 88031024 |
| 56 | NMD exon | ACTTTCACATCACCATGA | chr4 | 88031011 | 88031029 |
| 57 | NMD exon | GTATCACTTTCACATCAC | chr4 | 88031016 | 88031034 |
| 58 | NMD exon | AATGTGTATCACTTTCAC | chr4 | 88031021 | 88031039 |
| 59 | NMD exon | TATAGAATGTGTATCACT | chr4 | 88031026 | 88031044 |
| 60 | NMD exon | GTTTCTATAGAATGTGTA | chr4 | 88031031 | 88031049 |
| 61 | NMD exon | GTGTGGTTTCTATAGAAT | chr4 | 88031036 | 88031054 |
| 62 | NMD exon | TGAAAGTGTGGTTTCTAT | chr4 | 88031041 | 88031059 |
| 63 | NMD exon | AAAATTGAAAGTGTGGTT | chr4 | 88031046 | 88031064 |
| 64 | NMD exon | AATTCAAAATTGAAAGTG | chr4 | 88031051 | 88031069 |
| 65 | NMD exon | ACCAAAATTCAAAATTGA | chr4 | 88031056 | 88031074 |
| 66 | NMD exon | AAAAGACCAAAATTCAAA | chr4 | 88031061 | 88031079 |
| 67 | NMD exon | CTGGAAAAAGACCAAAAT | chr4 | 88031066 | 88031084 |
| 68 | NMD exon | GTAGCCTGGAAAAAGACC | chr4 | 88031071 | 88031089 |
| 69 | NMD exon | GTATGGTAGCCTGGAAAA | chr4 | 88031076 | 88031094 |
| 70 | NMD exon | TTAGAGTATGGTAGCCTG | chr4 | 88031081 | 88031099 |
| 71 | NMD exon | TCTTTAGAGTATGGTAGC | chr4 | 88031084 | 88031102 |
| 72 | NMD exon | CTCTATCTTTAGAGTATG | chr4 | 88031089 | 88031107 |
| 73 | NMD exon | TGTGGCTCTATCTTTAGA | chr4 | 88031094 | 88031112 |
| 74 | NMD exon | GGATCTGTGGCTCTATCT | chr4 | 88031099 | 88031117 |
| 75 | NMD exon | GACTGGGATCTGTGGCTC | chr4 | 88031104 | 88031122 |
| 76 | NMD exon | ACATGGCTGACTGGGATC | chr4 | 88031112 | 88031130 |
| 77 | NMD exon | TAATCACATGGCTGACTG | chr4 | 88031117 | 88031135 |
| 78 | NMD exon | CCTCATAATCACATGGCT | chr4 | 88031122 | 88031140 |
| 79 | NMD exon | TACCCTCATAATCACATG | chr4 | 88031125 | 88031143 |
| 80 | NMD exon | TCGCTTACCCTCATAATC | chr4 | 88031130 | 88031148 |
| 81 | NMD exon | ATTGGTCGCTTACCCTCA | chr4 | 88031135 | 88031153 |
| 82 | NMD exon | AGAGTATTGGTCGCTTAC | chr4 | 88031140 | 88031158 |
| 83 | NMD exon | ACTGTAGAGTATTGGTCG | chr4 | 88031145 | 88031163 |
| 84 | NMD exon | AATACACTGTAGAGTATT | chr4 | 88031150 | 88031168 |
| 85 | NMD exon | AATACAATACACTGTAGA | chr4 | 88031155 | 88031173 |
| 86 | NMD exon | CTGGCAATACAATACACT | chr4 | 88031160 | 88031178 |
| 87 | NMD exon | ACCATCTGGCAATACAAT | chr4 | 88031165 | 88031183 |
| 88 | NMD exon | GCAAAACCATCTGGCAAT | chr4 | 88031170 | 88031188 |
| 89 | NMD exon | GTTGGGCAAAACCATCTG | chr4 | 88031175 | 88031193 |
| 90 | NMD exon | GGCTAGTTGGGCAAAACC | chr4 | 88031180 | 88031198 |
| 91 | NMD exon | CATTAGGCTAGTTGGGCA | chr4 | 88031185 | 88031203 |
| 92 | NMD exon | ACTTACATTAGGCTAGTT | chr4 | 88031190 | 88031208 |
| 93 | NMD exon | AGAATACTTACATTAGGC | chr4 | 88031195 | 88031213 |
| 94 | NMD exon | TGTTTAGAATACTTACAT | chr4 | 88031200 | 88031218 |
| 95 | NMD exon | AAACATGTTTAGAATACT | chr4 | 88031205 | 88031223 |
| 96 | NMD exon | ACCTTAAACATGTTTAGA | chr4 | 88031210 | 88031228 |
| 97 | NMD exon | GGCCTACCTTAAACATGT | chr4 | 88031215 | 88031233 |
| 98 | NMD exon | AGCCTGGCCTACCTTAAA | chr4 | 88031220 | 88031238 |
| 99 | NMD exon | AGCTTAGCCTGGCCTACC | chr4 | 88031225 | 88031243 |
| 100 | uORF | TTTCTTCCCGGCGCCCGC | chr4 | 88007642 | 88007660 |
| 101 | uORF | CCTTTCTTCCCGGCGCCC | chr4 | 88007644 | 88007662 |
| 102 | uORF | TTCCTTTCTTCCCGGCGC | chr4 | 88007646 | 88007664 |
| 103 | uORF | TGTTCCTTTCTTCCCGGC | chr4 | 88007648 | 88007666 |
| 104 | uORF | CATGTTCCTTTCTTCCCG | chr4 | 88007650 | 88007668 |
| 105 | uORF | GCCATGTTCCTTTCTTCC | chr4 | 88007652 | 88007670 |
| 106 | uORF | GAGCCATGTTCCTTTCTT | chr4 | 88007654 | 88007672 |
| 107 | uORF | AGGAGCCATGTTCCTTTC | chr4 | 88007656 | 88007674 |
| 108 | uORF | TCAGGAGCCATGTTCCTT | chr4 | 88007658 | 88007676 |
| 109 | uORF | CCTCAGGAGCCATGTTCC | chr4 | 88007660 | 88007678 |
| 110 | uORF | CGCCTCAGGAGCCATGTT | chr4 | 88007662 | 88007680 |
| 111 | uORF | TGCGCCTCAGGAGCCATG | chr4 | 88007664 | 88007682 |
| 112 | uORF | TGTGCGCCTCAGGAGCCA | chr4 | 88007666 | 88007684 |
| 113 | uORF | CTGTGCGCCTCAGGAGCC | chr4 | 88007667 | 88007685 |
| 114 | Alt 5′ss | GUACUUUCAAAGUUAUUU | chr4 | 88036361 | 88036379 |
| 115 | Alt 5′ss | GAGAGGUACUUUCAAAGU | chr4 | 88036366 | 88036384 |
| 116 | Alt 5′ss | UGAUAGAGAGGUACUUUC | chr4 | 88036371 | 88036389 |
| 117 | Alt 5′ss | UUCUGUGAUAGAGAGGUA | chr4 | 88036376 | 88036394 |
| 118 | Alt 5′ss | CAAUUUUCUGUGAUAGAG | chr4 | 88036381 | 88036399 |
| 119 | Alt 5′ss | AUGAACAAUUUUCUGUGA | chr4 | 88036386 | 88036404 |
| 120 | Alt 5′ss | GCCAAAUGAACAAUUUUC | chr4 | 88036391 | 88036409 |
| 121 | Alt 5′ss | AUGAAGCCAAAUGAACAA | chr4 | 88036396 | 88036414 |
| 122 | Alt 5′ss | AAAUGAUGAAGCCAAAUG | chr4 | 88036401 | 88036419 |
| 123 | Alt 5′ss | CAUUGAAAUGAUGAAGCC | chr4 | 88036406 | 88036424 |
| 124 | Alt 5′ss | UCAUGCAUUGAAAUGAUG | chr4 | 88036411 | 88036429 |
| 125 | Alt 5′ss | GAUACUCAUGCAUUGAAA | chr4 | 88036416 | 88036434 |
| 126 | Alt 5′ss | CUGUCGAUACUCAUGCAU | chr4 | 88036421 | 88036439 |
| 127 | Alt 5′ss | AGGUCCUGUCGAUACUCA | chr4 | 88036426 | 88036444 |
| 128 | Alt 5′ss | AAAGCAGGUCCUGUCGAU | chr4 | 88036431 | 88036449 |
| 129 | Alt 5′ss | AAUGCAAAGCAGGUCCUG | chr4 | 88036436 | 88036454 |
| 130 | Alt 5′ss | UGUUAAAUGCAAAGCAGG | chr4 | 88036441 | 88036459 |
| 131 | Alt 5′ss | CACAGUGUUAAAUGCAAA | chr4 | 88036446 | 88036464 |
| 132 | Alt 5′ss | UCUCACACAGUGUUAAAU | chr4 | 88036451 | 88036469 |
| 133 | Alt 5′ss | UUACGUCUCACACAGUGU | chr4 | 88036456 | 88036474 |
| 134 | Alt 5′ss | AUAACUUACGUCUCACAC | chr4 | 88036461 | 88036479 |
| 135 | Alt 5′ss | UCACCAUAACUUACGUCU | chr4 | 88036466 | 88036484 |
| 136 | Alt 5′ss | ACAACUCACCAUAACUUA | chr4 | 88036471 | 88036489 |
| 137 | Alt 5′ss | UUCUAACAACUCACCAUA | chr4 | 88036476 | 88036494 |
| 138 | Alt 5′ss | GUAACUUCUAACAACUCA | chr4 | 88036481 | 88036499 |
| 139 | Alt 5′ss | GAACAGUAACUUCUAACA | chr4 | 88036486 | 88036504 |
| 140 | Alt 5′ss | AGUAGGAACAGUAACUUC | chr4 | 88036491 | 88036509 |
| 141 | Alt 5′ss | UUGAGAGUAGGAACAGUA | chr4 | 88036496 | 88036514 |
| 142 | Alt 5′ss | ACCCCCUUUGAGAGUAGG | chr4 | 88036503 | 88036521 |
| 143 | Alt 5′ss | AGUUUACCCCCUUUGAGA | chr4 | 88036508 | 88036526 |
| 144 | Alt 5′ss | AUGUUAGUUUACCCCCUU | chr4 | 88036513 | 88036531 |
| 145 | Alt 5′ss | UCUCAAUGUUAGUUUACC | chr4 | 88036518 | 88036536 |
| 146 | Alt 5′ss | AAAGUUCUCAAUGUUAGU | chr4 | 88036523 | 88036541 |
| 147 | Alt 5′ss | CAGGCAAAGUUCUCAAUG | chr4 | 88036528 | 88036546 |
| 148 | Alt 5′ss | AGGCACAGGCAAAGUUCU | chr4 | 88036533 | 88036551 |
| 149 | Alt 5′ss | GUGCAAGGCACAGGCAAA | chr4 | 88036538 | 88036556 |
| 150 | Alt 5′ss | GCACAGUGCAAGGCACAG | chr4 | 88036543 | 88036561 |
| 151 | Alt 5′ss | ACUCAGCACAGUGCAAGG | chr4 | 88036548 | 88036566 |
| 152 | Alt 5′ss | GAAACACUCAGCACAGUG | chr4 | 88036553 | 88036571 |
| 153 | Alt 5′ss | GAUAUGAAACACUCAGCA | chr4 | 88036558 | 88036576 |
| 154 | Alt 5′ss | GGUAAGAUAUGAAACACU | chr4 | 88036563 | 88036581 |
| 155 | Alt 5′ss | AAUAAGGUAAGAUAUGAA | chr4 | 88036568 | 88036586 |
| 156 | Alt 5′ss | AAUUAAAUAAGGUAAGAU | chr4 | 88036573 | 88036591 |
| 157 | Alt 5′ss | AUAGAAAUUAAAUAAGGU | chr4 | 88036578 | 88036596 |
| 158 | Alt 5′ss | AGACUAUAGAAAUUAAAU | chr4 | 88036583 | 88036601 |
| 159 | Alt 5′ss | GAGUUAGACUAUAGAAAU | chr4 | 88036588 | 88036606 |
| 160 | Alt 5′ss | UUAUAGAGUUAGACUAUA | chr4 | 88036593 | 88036611 |
| 161 | Alt 5′ss | UUACCUUAUAGAGUUAGA | chr4 | 88036598 | 88036616 |
| 162 | Alt 5′ss | AGUACUUACCUUAUAGAG | chr4 | 88036603 | 88036621 |
| 163 | NMD exon | AUAAAUCGAACCAUCGUA | chr4 | 88030981 | 88030999 |
| 164 | NMD exon | AAAUCAUAAAUCGAACCA | chr4 | 88030986 | 88031004 |
| 165 | NMD exon | UUGAAAAAUCAUAAAUCG | chr4 | 88030991 | 88031009 |
| 166 | NMD exon | UGAAGUUGAAAAAUCAUA | chr4 | 88030996 | 88031014 |
| 167 | NMD exon | CACCAUGAAGUUGAAAAA | chr4 | 88031001 | 88031019 |
| 168 | NMD exon | CACAUCACCAUGAAGUUG | chr4 | 88031006 | 88031024 |
| 169 | NMD exon | ACUUUCACAUCACCAUGA | chr4 | 88031011 | 88031029 |
| 170 | NMD exon | GUAUCACUUUCACAUCAC | chr4 | 88031016 | 88031034 |
| 171 | NMD exon | AAUGUGUAUCACUUUCAC | chr4 | 88031021 | 88031039 |
| 172 | NMD exon | UAUAGAAUGUGUAUCACU | chr4 | 88031026 | 88031044 |
| 173 | NMD exon | GUUUCUAUAGAAUGUGUA | chr4 | 88031031 | 88031049 |
| 174 | NMD exon | GUGUGGUUUCUAUAGAAU | chr4 | 88031036 | 88031054 |
| 175 | NMD exon | UGAAAGUGUGGUUUCUAU | chr4 | 88031041 | 88031059 |
| 176 | NMD exon | AAAAUUGAAAGUGUGGUU | chr4 | 88031046 | 88031064 |
| 177 | NMD exon | AAUUCAAAAUUGAAAGUG | chr4 | 88031051 | 88031069 |
| 178 | NMD exon | ACCAAAAUUCAAAAUUGA | chr4 | 88031056 | 88031074 |
| 179 | NMD exon | AAAAGACCAAAAUUCAAA | chr4 | 88031061 | 88031079 |
| 180 | NMD exon | CUGGAAAAAGACCAAAAU | chr4 | 88031066 | 88031084 |
| 181 | NMD exon | GUAGCCUGGAAAAAGACC | chr4 | 88031071 | 88031089 |
| 182 | NMD exon | GUAUGGUAGCCUGGAAAA | chr4 | 88031076 | 88031094 |
| 183 | NMD exon | UUAGAGUAUGGUAGCCUG | chr4 | 88031081 | 88031099 |
| 184 | NMD exon | UCUUUAGAGUAUGGUAGC | chr4 | 88031084 | 88031102 |
| 185 | NMD exon | CUCUAUCUUUAGAGUAUG | chr4 | 88031089 | 88031107 |
| 186 | NMD exon | UGUGGCUCUAUCUUUAGA | chr4 | 88031094 | 88031112 |
| 187 | NMD exon | GGAUCUGUGGCUCUAUCU | chr4 | 88031099 | 88031117 |
| 188 | NMD exon | GACUGGGAUCUGUGGCUC | chr4 | 88031104 | 88031122 |
| 189 | NMD exon | ACAUGGCUGACUGGGAUC | chr4 | 88031112 | 88031130 |
| 190 | NMD exon | UAAUCACAUGGCUGACUG | chr4 | 88031117 | 88031135 |
| 191 | NMD exon | CCUCAUAAUCACAUGGCU | chr4 | 88031122 | 88031140 |
| 192 | NMD exon | UACCCUCAUAAUCACAUG | chr4 | 88031125 | 88031143 |
| 193 | NMD exon | UCGCUUACCCUCAUAAUC | chr4 | 88031130 | 88031148 |
| 194 | NMD exon | AUUGGUCGCUUACCCUCA | chr4 | 88031135 | 88031153 |
| 195 | NMD exon | AGAGUAUUGGUCGCUUAC | chr4 | 88031140 | 88031158 |
| 196 | NMD exon | ACUGUAGAGUAUUGGUCG | chr4 | 88031145 | 88031163 |
| 197 | NMD exon | AAUACACUGUAGAGUAUU | chr4 | 88031150 | 88031168 |
| 198 | NMD exon | AAUACAAUACACUGUAGA | chr4 | 88031155 | 88031173 |
| 199 | NMD exon | CUGGCAAUACAAUACACU | chr4 | 88031160 | 88031178 |
| 200 | NMD exon | ACCAUCUGGCAAUACAAU | chr4 | 88031165 | 88031183 |
| 201 | NMD exon | GCAAAACCAUCUGGCAAU | chr4 | 88031170 | 88031188 |
| 202 | NMD exon | GUUGGGCAAAACCAUCUG | chr4 | 88031175 | 88031193 |
| 203 | NMD exon | GGCUAGUUGGGCAAAACC | chr4 | 88031180 | 88031198 |
| 204 | NMD exon | CAUUAGGCUAGUUGGGCA | chr4 | 88031185 | 88031203 |
| 205 | NMD exon | ACUUACAUUAGGCUAGUU | chr4 | 88031190 | 88031208 |
| 206 | NMD exon | AGAAUACUUACAUUAGGC | chr4 | 88031195 | 88031213 |
| 207 | NMD exon | UGUUUAGAAUACUUACAU | chr4 | 88031200 | 88031218 |
| 208 | NMD exon | AAACAUGUUUAGAAUACU | chr4 | 88031205 | 88031223 |
| 209 | NMD exon | ACCUUAAACAUGUUUAGA | chr4 | 88031210 | 88031228 |
| 210 | NMD exon | GGCCUACCUUAAACAUGU | chr4 | 88031215 | 88031233 |
| 211 | NMD exon | AGCCUGGCCUACCUUAAA | chr4 | 88031220 | 88031238 |
| 212 | NMD exon | AGCUUAGCCUGGCCUACC | chr4 | 88031225 | 88031243 |
| 213 | uORF | UUUCUUCCCGGCGCCCGC | chr4 | 88007642 | 88007660 |
| 214 | uORF | CCUUUCUUCCCGGCGCCC | chr4 | 88007644 | 88007662 |
| 215 | uORF | UUCCUUUCUUCCCGGCGC | chr4 | 88007646 | 88007664 |
| 216 | uORF | UGUUCCUUUCUUCCCGGC | chr4 | 88007648 | 88007666 |
| 217 | uORF | CAUGUUCCUUUCUUCCCG | chr4 | 88007650 | 88007668 |
| 218 | uORF | GCCAUGUUCCUUUCUUCC | chr4 | 88007652 | 88007670 |
| 219 | uORF | GAGCCAUGUUCCUUUCUU | chr4 | 88007654 | 88007672 |
| 220 | uORF | AGGAGCCAUGUUCCUUUC | chr4 | 88007656 | 88007674 |
| 221 | uORF | UCAGGAGCCAUGUUCCUU | chr4 | 88007658 | 88007676 |
| 222 | uORF | CCUCAGGAGCCAUGUUCC | chr4 | 88007660 | 88007678 |
| 223 | uORF | CGCCUCAGGAGCCAUGUU | chr4 | 88007662 | 88007680 |
| 224 | uORF | UGCGCCUCAGGAGCCAUG | chr4 | 88007664 | 88007682 |
| 225 | uORF | UGUGCGCCUCAGGAGCCA | chr4 | 88007666 | 88007684 |
| 226 | uORF | CUGUGCGCCUCAGGAGCC | chr4 | 88007667 | 88007685 |

TABLE 6
Exemplary Mouse U7 vector sequence

| Region | Sequence |
|---|---|
| Promoter | cccacaucgccugccacuacuuaaguccgauucacuucggcuuuagcuccaagccuuuaaucucgcgaag |
| sequence (Mouse | cucuuuuuuuuuuuuuaacaacauaggagcugugauuggcguuuucagccaaucagcacugacucau |
| U7 promoter) | uugcauagccuuuacaagoggucacaaacucaagaaacgagcgguuuuaauagucuuuuagaauauugu |
| | uuaucgaaccgaauaaggaacugugcuuugugauucacauaucaguggagggguguggaaauggcacc |
| | uugaucucacccucaucgaaaguggaguugauguccuucccuggcucguacagageacuuccgc |
| | (SEQ ID NO: 252) |
| Wild-type U7 | AAGUGUUACAGCUCUUUUAG (SEQ ID NO: 253) |
| Antisense | |
| sequence | |
| Wild-type U7 | AAUUUGUCUAGCAGGUUUUCUGACUUCGGUCGGAAAACCCCU (SEQ |
| non-coding RNA | ID NO: 254) |
| sequence | |
| ASO sequences | GTACTTTCAAAGTTATTT (SEQ ID NO: 1) |
| replacing the | ATAAATCGAACCATCGTA (SEQ ID NO: 50) |
| Wild-type U7 | TTTCTTCCCGGCGCCCGC (SEQ ID NO: 100) |
| Antisense | |
| sequence | |
| Modified U7 | AAUUUUUGGAGCAGGUUUUCUGACUUCGGUCGGAAAACCCCU (SEQ |
| snRNA (smOPT) | ID NO: 255) |
| non-coding RNA | |
| sequence | |
| smOPT sequence | AAUUUUUGGAG (SEQ ID NO: 229) |
| 3′ regulatory | cccaauuucacuggucuacaaugaaagcaaaacaguucucuuccccgcuccccggugugugagaggggc |
| sequence | uuugauccuucucugguuuccuaggaaacgcguaugugcuagagccacgcucugagacuuccgccucgu |
| | gcggucccgcuuccuuucugccuccucugg (SEQ ID NO: 256) |
| Exemplary Full | cccacaucgccugccacuacuuaaguccgauucacuucggcuuuagcuccaagccuuuaaucucgcgaag |
| sequence of wild- | cucuuuuuuuuuuuuuaacaacauaggagcugugauuggcuguuuucagccaaucagcacugacucau |
| type U7 snRNA | uugcauagccuuuacaagoggucacaaacucaagaaacgagogguuuuaauagucuuuuagaauauugu |
| | uuaucgaaccgaauaaggaacugugcuuugugauucacauaucaguggagggguguggaaauggcacc |
| | uugaucucacccucaucgaaaguggaguugauguccuucccuggcucgcuacagacgcacuuccgcAA |
| | GUGUUACAGCUCUUUUAGAAUUUGUCUAGCAGGUUUUCUGACUUC |
| | GGUCGGAAAACCCCUcccaauuucacuggucuacaaugaaagcaaaacaguucucuuccccg |
| | cuccccggugugugagaggggcuuugauccuucucugguuuccuaggaaagcguauguguagagcc |
| | acgcucugagacuuccgccucgugcggucccgcuucuuucugccuccucugg (SEQ ID NO: |
| | 257) |
| Exemplary Full | cccacaucgccugccacuacuuaaguccgauucacuucggcuuuagcuccaagccuuuaaucucgcgaag |
| sequence of | cucuuuuuuuuuuuuuaacaacauaggagugugauuggcuguuuucacaucagacugacucau |
| modified U7 | uugcauagccuuuacaagoggucacaaacucaagaaacgagcgguuuuaauagucuuuuagaauauugu |
| snRNA (smOPT) | uuaucgaaccgaauaaggaacugugcuuugugauucacauaucaguggagggguguggaaauggcacc |
| | uugaucucacccucaucgaaaguggaguugauguccuucccuggcucgcuacagacgcacuuccgcAA |
| | GUGUUACAGCUCUUUUAGAAUUUUUGGAGCAGGUUUUCUGACUUC |
| | GGUCGGAAAACCCCUcccaauuucacuggucuacaaugaaagcaaaacaguucucuuccccg |
| | cuccccggugugugagaggggcuuugauccuucucugguuuccuaggaaacgcguaugugcuagagcc |
| | acgcucugagacuuccgccucgugcggucccgcuuccuuucugccuccucugg (SEQ ID NO: |
| | 258) |
| Full sequence of | cccacaucgccugccacuacuuaaguccgauucacuucggcuuuagcuccaagccuuuaaucucgcgaag |
| modified U7 | cucuuuuuuuuuuuuuaacaacauaggagcugugauuggcuguuuucagccaaucagcacugacucau |
| snRNA (smOPT) | uugcauagccuuuacaagoggucacaaacucaagaaacgagcgguuuuaauagucuuuuagaauauugu |
| containing an | uuaucgaaccgaauaaggaacugugcuuugugauucacauaucaguggagggguguggaaauggcacc |
| ASO sequence | uugaucucacccucaucgaaaguggaguugauguccuucccuggcucgcuacagacgcacuuccgc[ASO |
| replacing the | sequence]AAUUUUUGGAGCAGGU |
| antisense | UUUCUGACUUCGGUCGGAAAACCCCUcccaauuucacuggucuacaaugaaagcaaa |
| sequence of U7 | acaguucucuuccccgcuccccggugugugagaggggcuuugauccuucucugguuuccuaggaaacg |
| snRNA | cguaugugcuagagccacgcucugagacuuccgccucgugcggucccgcuuccuuucugccuccucugg |
| (Exemplary | (SEQ ID NOS 252 and 259, respectively) |
| sequences of [ASO | |
| sequence] include | |
| those in Table 4 | |
| and Table 5) | |
| Exemplary full | cccacaucgccugccacuacuuaaguccgauucacuucggcuuuagcuccaagccuuuaaucucgcgaag |
| sequence of | cucuuuuuuuuuuuuuaacaacauaggagugugauuggcuguuuucagccaaucagcacugacucau |
| modified U7 | uugcauagccuuuacaagcggucacaaacucaagaaacgagcgguuuuaauagucuuuuagaauauugu |
| snRNA (smOPT) | uuaucgaaccgaauaaggaacugugcuuugugauucacauaucaguggagggguguggaaauggcacc |
| containing an | uugaucucacccucaucgaaaguggaguugauguccuucccuggcucgcuacagacgcacuuccgcGU |
| ASO sequence | ACUUUCAAAGUUAUUUAAUUUUUGGAGCAGGUUUUCUGACUUCGG |
| replacing the | UCGGAAAACCCCUcccaauuucacuggucuacaaugaaagcaaaacaguucucuuccccgcuc |
| antisense | cccggugugugagaggggcuuugauccuucucugguuuccuaggaaacgcguaugugcuagagccacg |
| sequence of U7 | cucugagacuuccgccucgugcggucccgcuuccuuucugccuccucugg (SEQ ID NO: 260) |
| snRNA | |
| Exemplary full | cccacaucgccugccacuacuuaaguccgauucacuucggcuuuagcuccaagccuuuaaucucgcgaag |
| sequence of | cucuuuuuuuuuuuuuaacaacauaggagcugugauuggcuguuuucagccaaucagcacugacucau |
| modified U7 | uugcauagccuuuacaagoggucacaaacucaagaaacgagcgguuuuaauagucuuuuagaauauugu |
| snRNA (smOPT) | uuaucgaaccgaauaaggaacugugcuuugugauucacauaucaguggagggguguggaaauggcacc |
| containing an | uugaucucacccucaucgaaaguggaguugauguccuucccuggcucgcuacagacgcacuuccgcAU |
| ASO sequence | AAAUCGAACCAUCGUAAAUUUUUGGAGCAGGUUUUCUGACUUCGG |
| replacing the | UCGGAAAACCCCUcccaauuucacuggucuacaaugaaagcaaaacaguucucuuccccgcuc |
| antisense | cccggugugugagaggggcuuugauccuucucugguuuccuaggaaacgcguaugugcuagagccacg |
| sequence of U7 | cucugagacuuccgccucgugcggucccgcuuccuuucugccuccucugg |
| snRNA | (SEQ ID NO: 261) |
| Exemplary full | cccacaucgccugccacuacuuaaguccgauucacuucggcuuuagcuccaagccuuuaaucucgcgaag |
| sequence of | cucuuuuuuuuuuuuuaacaacauaggagugugauuggcuguuuucagcaucagacugacucau |
| modified U7 | uugcauagccuuuacaagoggucacaaacucaagaaacgagcgguuuuaauagucuuuuagaauauugu |
| snRNA (smOPT) | uuaucgaaccgaauaaggaacugugcuuugugauucacauaucaguggagggguguggaaauggcacc |
| containing an | uugaucucacccucaucgaaaguggaguugauguccuucccuggcucguacagacgcacuuccgcUU |
| ASO sequence | UCUUCCCGGCGCCCGCAAUUUUUGGAGCAGGUUUUCUGACUUCGG |
| replacing the | UCGGAAAACCCCUcccaauuucacuggucuacaaugaaagcaaaacaguucucuuccccgcuc |
| antisense | cccggugugugagaggggcuuugauccuucucugguuuccuaggaaacgcguaugugcuagagccacg |
| sequence of U7 | cucugagacuuccgccucgugcggucccgcuuccuuucugccuccucugg (SEQ ID NO: 262) |
| snRNA | |

### EXAMPLES

The present disclosure will be more specifically illustrated by the following Examples. However, it should be understood that the present disclosure is not limited by these examples in any manner.

### Example 1: Identification of NMD-Inducing Exon Inclusion Events in Transcripts by RNAseq Using Next Generation Sequencing

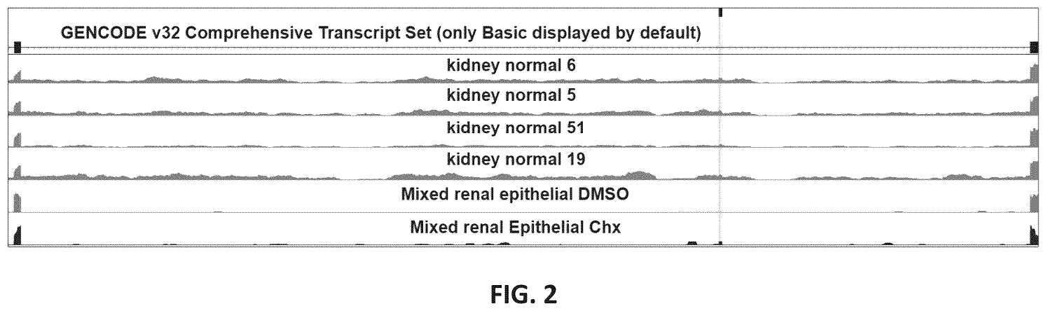

Whole transcriptome shotgun sequencing was carried out using next generation sequencing to reveal a snapshot of transcripts produced by the genes described herein to identify NIE inclusion events. For this purpose, polyA+ RNA from nuclear and cytoplasmic fractions of human cells was isolated and cDNA libraries were constructed using Illumina's TruSeq Stranded mRNA library Prep Kit. The libraries are pair-end sequenced resulting in 100-nucleotide reads that are mapped to the human genome (Grch38/hg38 assembly). FIG. 2 depicts identification of different exemplary nonsense-mediated mRNA decay (NMD)-inducing exons in PKD2 gene.

### Example 2: Confirmation of NIE Via Cycloheximide Treatment

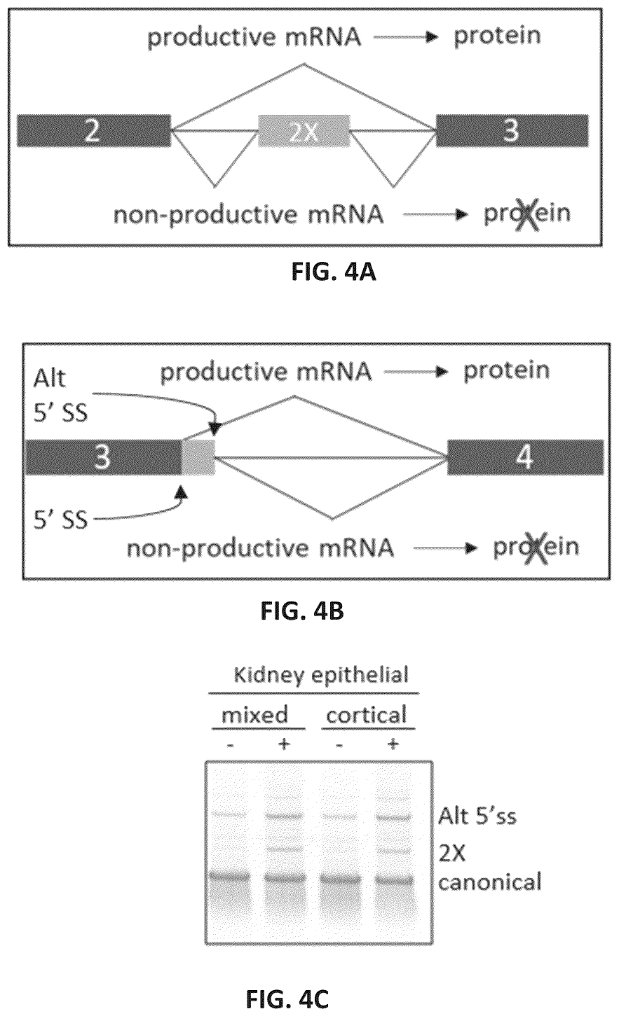

RT-PCR analysis using cytoplasmic RNA from DMSO-treated or cycloheximide-treated human kidney mixed epithelial cells and human kidney cortical epithelial cells and primers in exons can confirm the presence of a band corresponding to an NMD-inducing exon. The identity of the product was confirmed by sequencing. Densitometry analysis of the bands was performed to calculate percent NMD exon inclusion of total transcript. Treatment of cells with cycloheximide to inhibit NMD can lead to an increase of the product corresponding to the NMD-inducing exon in the cytoplasmic fraction. FIG. 4B depicts confirmation of exemplary NIE exons in various gene transcripts using cycloheximide treatment, respectively.

### Example 3: NMD Exon Region ASO Walk

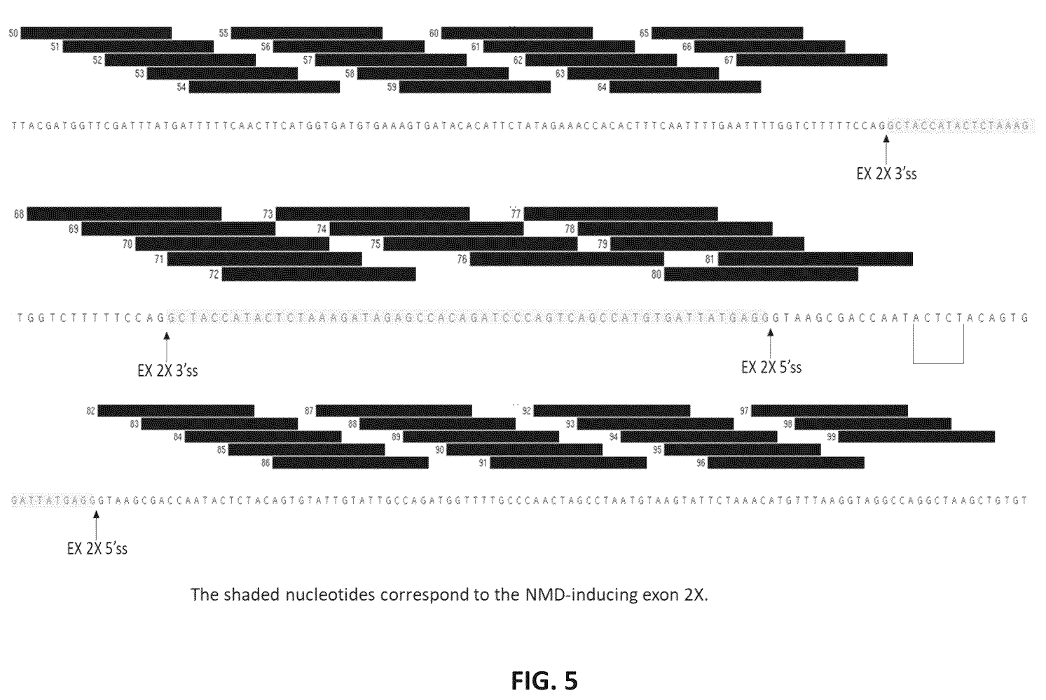

An ASO walk was performed for NMD exon region targeting sequences immediately upstream of the 3′ splice site, across the 3′splice site, the NMD exon, across the 5′ splice site, and downstream of the 5′ splice site using ASOs. ASOs are designed to cover these regions by shifting 5 nucleotides at a time. FIG. 5 depicts ASO walk for an exemplary NIE exon region.

### Example 4: NMD Exon Region ASO Walk Evaluated by RT-PCR

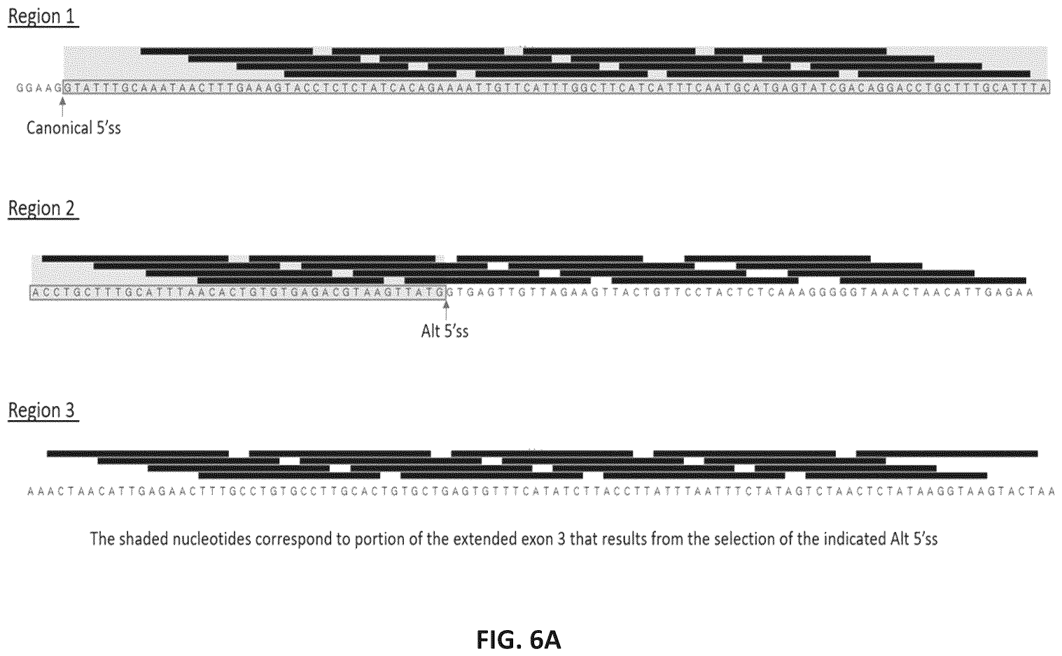

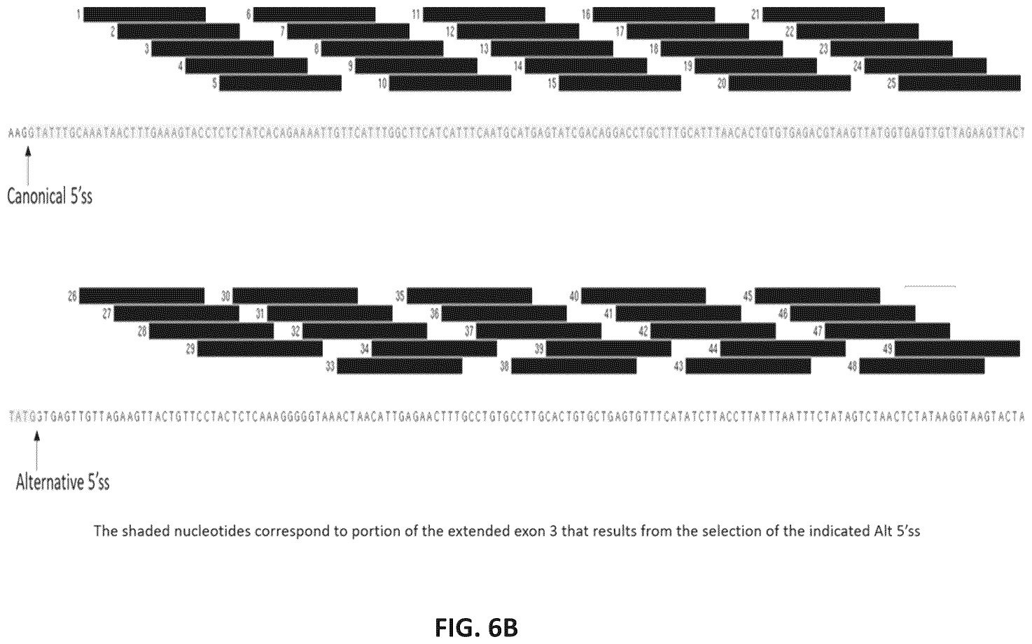

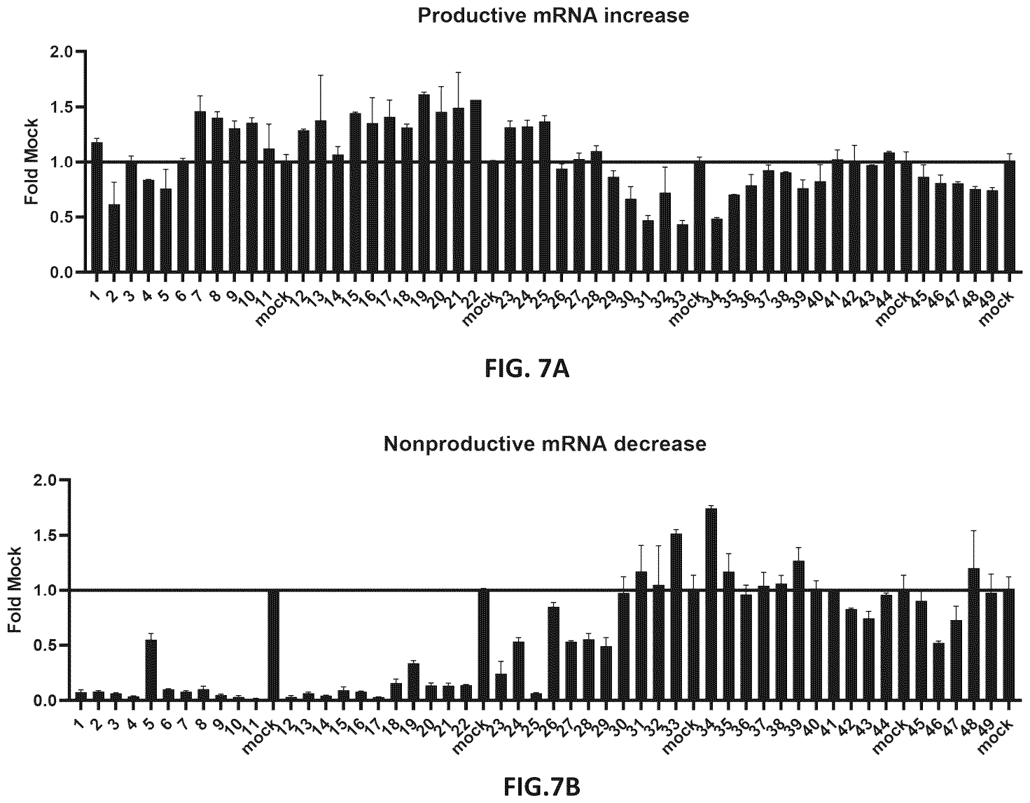

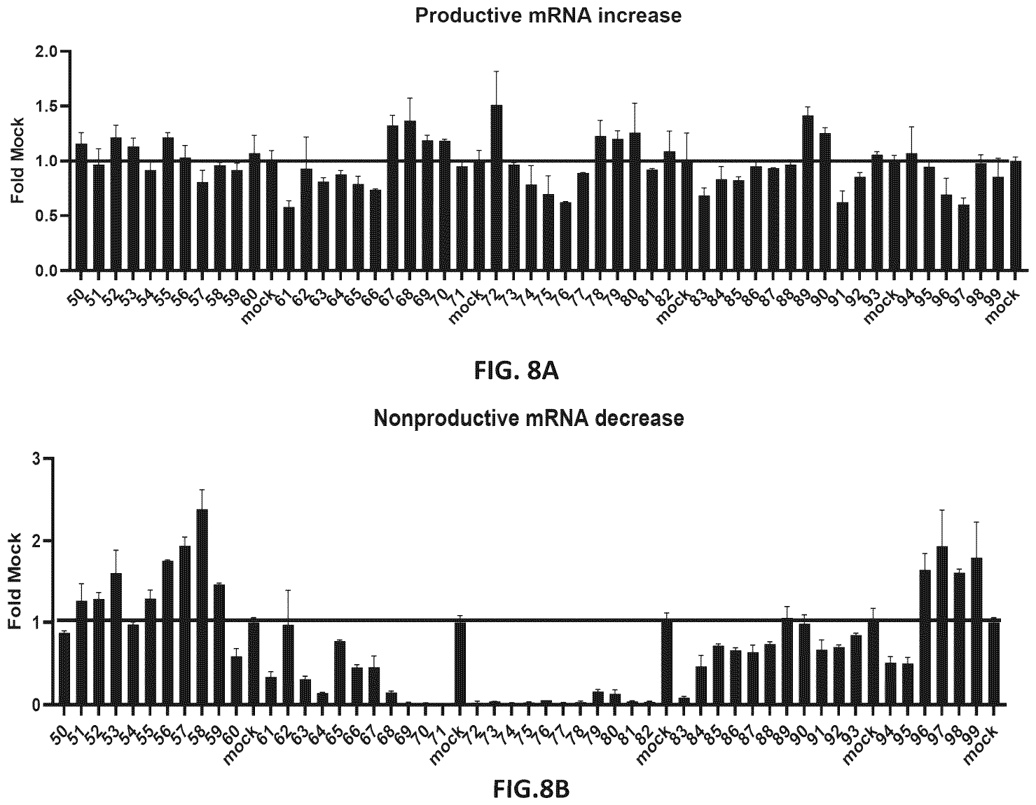

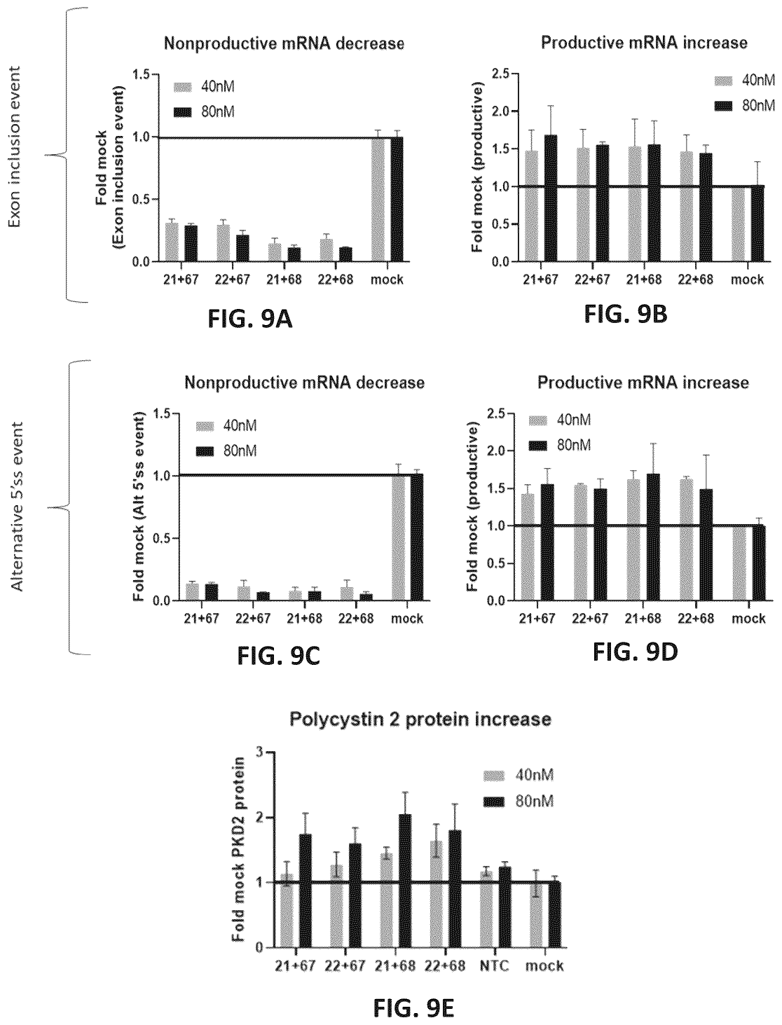

ASO walk sequences can be evaluated by for example RT-PCR. PAGE can be used to show SYBR-safe-stained RT-PCR products of human cells treated with a ASO targeting the NMD exon regions as described herein in human cells by gymnotic uptake, transfection or nucleofection. Products corresponding to NMD exon inclusion and full-length are quantified and percent NMD exon inclusion is plotted. Full-length products can be normalized to internal mRNA controls and fold-change relative to control can be plotted. FIG. 7A depicts the changes in productive PKD2 mRNA as evaluated by probe-based RT-qPCR (see Example 5, normalized to RPL32) using primary renal mixed epithelial cells transfected for 24 hours with the indicated ASOs (see macrowalk of FIGS. 6A-B) at 80 nM. FIG. 7B depicts the changes in nonproductive PKD2 mRNA as evaluated by probe-based RT-qPCR (see Example 5, normalized to RPL32) using primary renal mixed epithelial cells transfected for 24 hours with the indicated ASOs (see macrowalk of FIGS. 6A-B) at 80 nM. FIG. 8A depicts the changes in productive PKD2 mRNA as evaluated by probe-based RT-qPCR (see Example 5, normalized to RPL32) using primary renal mixed epithelial cells transfected for 24 hours with the indicated ASOs (see macrowalk of FIG. 5) at 80 nM. FIG. 8B. depicts the changes in nonproductive PKD2 mRNA as evaluated by probe-based RT-qPCR (see Example 5, normalized to RPL32) using primary renal mixed epithelial cells transfected for 24 hours with the indicated ASOs (see macrowalk of FIG. 5) at 80 nM.

### Example 5: NMD Exon Region ASO Walk Evaluated by RT-qPCR

SYBR-green or any probe-based RT-qPCR amplification results normalized to the internal mRNA control can be obtained using the same ASO uptake experiment that can be evaluated by RT-qPCR and can be plotted as fold change relative to Sham to confirm RT-qPCR results.

### Example 6: Dose-Dependent Effect of Selected ASO in CXH-Treated Cells

PAGE can be used to show SYBR-safe-stained RT-PCR products of mock-treated (Sham, RNAiMAX alone), or treated with ASOs targeting NMD exons at 30 nM, 80 nM, and 200 nM concentrations in mouse or human cells by RNAiMAX transfection. Products corresponding to NMD exon inclusion and full-length are quantified and percent NMD exon inclusion can be plotted. The full-length products can also be normalized to HPRT internal control and fold-change relative to Sham can be plotted.

### Example 7: Injection of Selected ASOs

PAGEs of SYBR-safe-stained RT-PCR products of mice from PBS-injected (1 μL) (−) or ASOs or Cep290 (negative control ASO; Gerard et al, Mol. Ther. Nuc. Ac., 2015) 2′-ASO-injected (1 μL) (+) at various concentration. Products corresponding to NMD exon inclusion and full-length are quantified and percent NMD exon inclusion can be plotted. Full-length products can be normalized to GAPDH internal control and fold-change of ASO-injected relative to PBS-injected can plotted.

### Example 8: Identification of NMD-Inducing Alternative Splicing Events in Transcripts by RNAseq Using Next Generation Sequencing

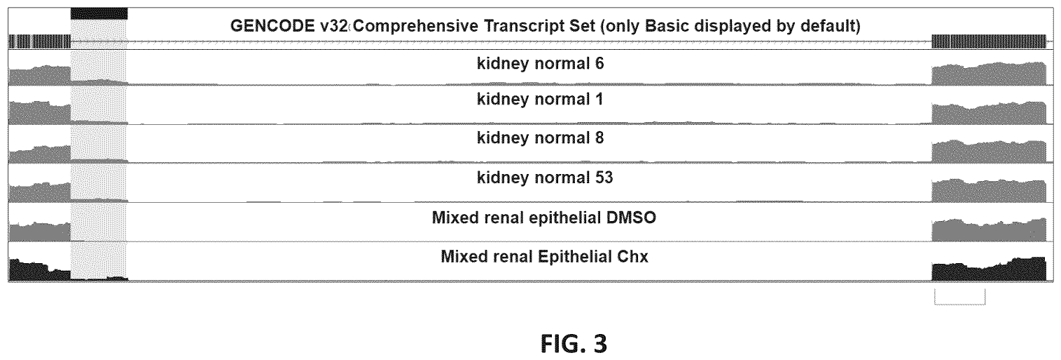

Non-productive AS events in organs known to be accessible by ASOs were identified by analyzing 83 publicly available RNA-sequencing (RNA-seq) datasets from human liver, kidney, central nervous system (CNS), and eye tissues. Computational analysis discovered 7,819 unique genes containing a total of 13,121 non-productive AS events of various types. By cross-referencing these genes with genetic disease databases such as Orphanet (www.orpha.net/), 1,265 disease-associated genes with non-productive AS events were identified. As many NMD-sensitive transcripts are efficiently degraded in the analyzed tissues and are not detectable by RNAseq, there are many more genes with non-productive AS events than have been identified to date. FIG. 3 depicts identification of different exemplary nonsense-mediated mRNA decay (NMD)-inducing alternative splicing events in PKD2 gene.

### Example 9: Confirmation of Alternative Splicing Via Cycloheximide Treatment

To validate the in-silico predictions and to quantify the abundance of potentially targetable non-productive AS events, cells were treated with cycloheximide (CHX), a translation inhibitor known to inhibit NMD. Expectedly, reverse transcriptase (RT)-PCR analysis showed a consistent increase in the predicted non-productive PKD2 splicing events in various cell lines upon CHX treatment compared to DMSO-treated cells. The increase indicates that these non-productive AS events lead to transcript degradation by NMD. FIG. 4B depicts confirmation of exemplary NIE exons in various gene transcripts using cycloheximide treatment, respectively Example 10: PKD2 Exon, alternative 5′ ss and 5′UTR Region ASO Walk

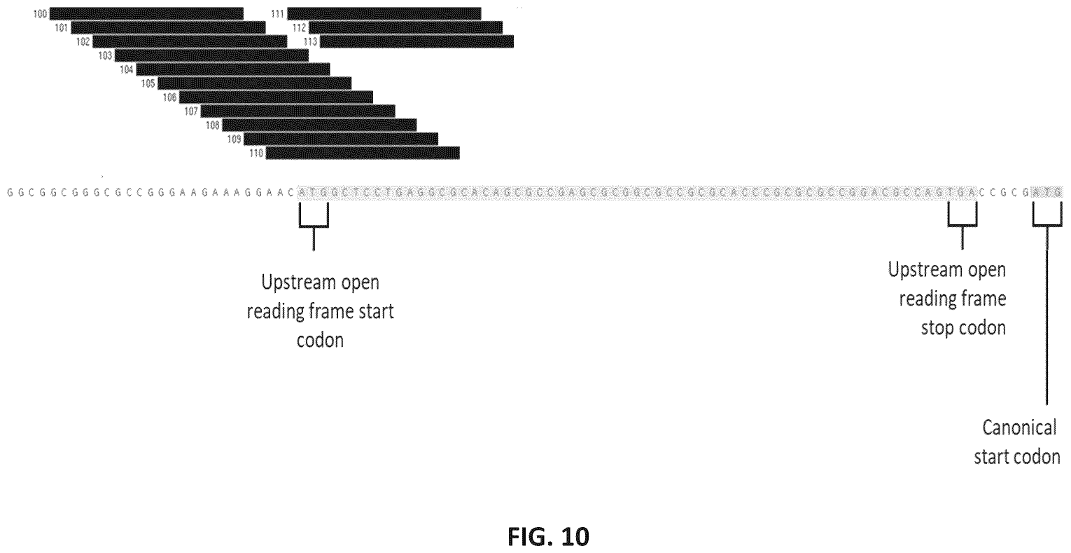

To identify ASOs that can prevent the non-productive AS events and/or modulate translation of a PKD2 mRNA transcript, an initial systematic ASO walk was performed in 5-nt steps along the AS event of interest or the 5′ UTR of PKD2. FIG. 5 shows a systematic ASO walk along an NMD exon AS event of PKD2 pre-mRNA. FIGS. 6A and 6B show a systematic ASO walk along an alternative 5′ ss AS event of PKD2 pre-mRNA. FIG. 10 shows a systematic ASO walk along a 5′ UTR of PKD2 mRNA. These ASOs may have a uniform phosphorothioate backbone and methoxyethyl at the 2′ ribose position (2′MOE-PS). These modifications were previously shown to allow binding to RNA with high affinity and to confer resistance to both nucleases and RNase H cleavage of the target RNA-ASO complex. RT-PCR analysis from transfected cells identifies several ASOs that reduce AS in the PKD2 mRNA and increase productive mRNA or promote translation of PKD2. The observed increase in PKD2 productive mRNA is confirmed by TaqMan qPCR. The observed increase in PKD2 translation is confirmed by Western blotting. The fold change of AS may be plotted vs the increase in productive mRNA (qPCR) to demonstrate that the ASOs are functioning on mechanism. The observed increase in PKD2 translation is confirmed by Western blotting. The fold change of translation may be plotted vs the increase in polycystin 2 protein (Western blotting) to demonstrate that the ASOs are functioning on mechanism. These results are expected to strongly suggest that gene expression upregulation can be achieved by preventing non-productive AS with ASOs.

### Example 11: Use of ASO's to Increase Cellular Protein Expression in a Dose-Dependent Manner

As the desirable upregulation level varies among target genes and diseases, selected positive ASO hits from the initial walks are used to determine whether the increase in productive mRNA can be titrated across non-productive AS events. An exemplary PKD2 ASO is transfected in cells at increasing concentrations to demonstrate dose-dependent upregulation. The concentration is selected based on the potency of the ASO. RT-PCR results show a dose-dependent decrease of the non-productive alternative 3′ss selection in PKD2 compared to a non-targeting ASO control transfected at the same respective doses. Conversely, a dose-dependent increase in productive mRNA is observed as measured by TaqMan qPCR compared to a non-targeting ASO control. To determine whether the observed upregulation in productive mRNAs translates to a dose-dependent increase in protein levels, polycystin 2 is measured in extracts from transfected cells with increasing concentrations of targeting ASOs. First, antibodies against PKD2 are validated by short interfering (si)RNA-mediated knockdown of protein expression and western blot analysis. Immunoblotting results of extracts from cells transfected with the selected ASOs are expected to show a dose-dependent increase in polycystin 2. A non-targeting ASO control is expected no significant effect on protein levels. Altogether, the data are expected to indicate that ASOs targeting various types of non-productive AS events lead to a titratable increase in productive mRNA resulting in an increase in protein expression. The titratable nature of TANGO ASO-mediated protein upregulation is expected to suggest that one could tightly control protein levels and reduce the risk of overexpression. This aspect of the TANGO technology is expected to make it especially suited to address autosomal dominant haploinsufficient diseases.

### Example 12: Validation of TANGO Approach In Vivo

To prove the applicability of the TANGO mechanism in vivo, one may select a positive hit from an ASO walk targeting a non-productive exon inclusion event in PKD2. The non-productive AS event in the human PKD2 gene also occurs in mice and is highly conserved at the sequence level (data not shown), allowing for testing the human targeting ASO in mice. Similar to other ASOs presented here, gymnotic (free) uptake of increasing concentrations of PKD2 ASO leads to a dose-dependent decrease of AS and an inversely correlated increase in productive mRNA in cells compared to a non-targeting ASO control. To ascertain whether the observed effect of the ASO can be recapitulated in vivo, administer ASO to mice or PBS to mice via injection. RNA and protein can be extracted from the treated mice 5 days post-injection. RT-PCR analysis can be done to show clear target engagement and a consistent reduction of non-productive exon inclusion in ASO-treated mice compared to the control PBS cohort. This reduction can be effectively translated to a roughly 4-fold increase in productive mRNA measured by TaqMan qPCR. Concomitantly, increases in protein by western blot can be detected using a validated antibody. These data provide in vivo proof of concept of the TANGO approach to upregulate protein expression by leveraging non-productive AS events.

### Example 13: TANGO (Targeted Augmentation of Nuclear Gene Output) for the Treatment of Genetic Diseases

TANGO (Targeted Augmentation of Nuclear Gene Output), a novel technology which exploits antisense-mediated modulation of pre-mRNA splicing was developed to increase protein expression. TANGO prevents naturally occurring non-productive splicing events that lead to either transcript degradation by nonsense-mediated mRNA decay (NMD) or nuclear retention. By doing so, TANGO increases the generation of productive mRNA, resulting in an increase of full-length, fully-functional protein. Bioinformatic analyses of RNA sequencing (RNAseq) datasets were undertaken to identify non-productive events. Non-productive events were found in more than 50% of protein-coding genes, of which approximately 2,900 are disease-associated. To validate the in-silico predictions, targets (e.g., PKD2) representing various types of NMD-inducing, non-productive alternative splicing (AS) events (cassette exons, alternative splice sites, and alternative introns) were selected and quantified their abundance by treating cells with cycloheximide (CHX), a translation inhibitor that is known to inhibit NMD. RT-PCR analyses of the selected targets was performed and an increase of the non-productive mRNA upon CHX treatment was observed compared to DMSO-treated cells. Antisense oligonucleotides (ASOs) are designed to target the three types of NMD-inducing, non-productive AS events and TANGO ASOs are expected to be able to modulate splicing to increase productive mRNA and protein in a dose-dependent manner in vitro. Consistent with the TANGO mechanism, the level of ASO-mediated upregulation is expected to be observed to be directly proportional to the abundance of the targeted NMD-inducing event. Moreover, injection in wild-type mice of a TANGO ASO targeting a non-productive AS event in PKD2 is expected to lead to an increase in productive mRNA and polycystin 2. As TANGO exploits naturally occurring non-productive AS, this novel approach can be employed to upregulate gene expression from wild-type or hypomorphic alleles, providing a potentially unique strategy to treat genetic diseases. TANGO is being applied to develop treatment for autosomal dominant haploinsufficiency diseases such as genetic epilepsies. TANGO ASOs that increase expression from the wild-type alleles can be used to restore physiological levels of the deficient proteins.

### Example 14: Transcript Database and Labeling of NMD Junctions

Annotated transcripts are downloaded from GENCODE (v. 28) and REFSEQ (via UCSC). Each annotated exon-exon junction is labeled as “coding” or “NMD”. Junctions are labeled “NMD” if and only if that junction is exclusively found in transcripts labeled “nonsense_mediated_decay” (GENCODE) or “NR” (REFSEQ).

### RNA-Seq Library Processing:

All RNA-seq samples are aligned to the hg38 genome and a combined transcript database using STAR¹ v2.6.1b to generate splice junction counts.

### Identification and Quantification of Putative NMD-Inducing Splicing Events:

All samples are run through SUPPA2² to define annotated alternative splicing events. Different approaches are then used to label and quantify each type of alternative splicing as follows:

Exon inclusion (EI) and exon skipping (ES): The “skipped exon” events are parsed from SUPPA to obtain the inclusion and skipping junctions for each event. If the skipping junction is labeled “NMD”, the event is labeled “ES_NMD”. If either of the inclusion junctions are labeled as “NMD”, the event is labeled “EI_NMD”. Otherwise, the event is labeled “cassette exon.” Inclusion and skipping junction counts are retrieved from the STAR output, and these counts are summed across all events sharing the same alternatively spliced exon.

The final PSI for the inclusion is calculated as:

$$Ψ = \frac{{sj}_{inc}}{{sj}_{inc} + 2 \cdot {sj}_{skip}} .$$

For inclusion events, Ψ_{EI_NMD}=Ψ.

For skipping events, Ψ_{ES_NMD}=1−Ψ.

Alternative 3′ and 5′ splice sites (A3 and A5): The A3 and A5 events are parsed from SUPPA to obtain the junctions corresponding to each alternative event. If either the long or short junction is labeled as “NMD”, an NMD event is reported. If both junctions report NMD it is not reported because there is likely complex splicing in that region. Splice junction counts are retrieved from the STAR output. The PSI is reported as

$${Ψ}_{A \{ 3 , 5 \} \_ NMD} = \frac{{sj}_{NMD}}{{sj}_{coding}} .$$

Alternative intron events (AI): The retained intron events are parsed from SUPPA to obtain the list of alternative intron events. The AI event is labeled NMD if the event junction is labeled NMD. To calculate PSI, the expression level of the exon within which the AI is located is estimated by summing all junctions using its 3′ and 5′ splice sites (and all other parent exons containing the same AI event). Usage of the AI junction will then fall within the range

$$[ 0 , ∼ \frac{{sj}_{{3}^{′}} + {sj}_{5 ′}}{2} ] ,$$

because full use of the AI junction (which results in no intron retention) is achieved with similar counts at the exon junctions and the AI junction (full intron retention has 0 reads for the junction). To calculate Ψ, the junction counts are normalized, such that this range would now be [0,1]

$${Ψ}_{AI} ∼ \frac{min ( {sj}_{AI ′} \frac{{sj}_{{3}^{′}} + {sj}_{5 ′}}{2} )}{\frac{{sj}_{{3}^{′}} + {sj}_{5 ′}}{2}} .$$

### Annotation of Disease Relevance:

Gene-disease association data from Orphadata (http://www.orphadata.org), the publicly available data repository of Orphanet is downloaded. The annotations were extended to cover all gene symbol aliases.

### Example 15: Treatment with Cycloheximide (CHX), Cell Culture, and Transfections

To determine the abundance of the non-productive mRNAs, cells are incubated with 50 μg/ml of CHX (Cell Signaling Technology) dissolved in DMSO for 3 hours.

For example, cells are grown in EMEM with 10% FBS and 1×10⁵ cells are seeded in 24-well plate and reverse-transfected with 80 nM ASOs for initial screening or 1, 5, 25 nM of selected ASO using Lipofectamine RNAiMax reagent (Invitrogen) according to manufacturer's instructions. Total RNA is extracted using RNeasy mini kit (Qiagen) 24 hrs post-transfection and cDNA is synthesized with ImProm-II reverse transcriptase (Promega). Total protein is extracted with RIPA buffer (Cell Signaling Technology) 48 hrs post transfection.

For another example, HEK293 cells are grown in EMEM with 10% FBS and 7×10⁵ cells are seeded in 6-well plate and reverse-transfected with 30, 60, and 120 nM of antisense oligonucleotide (ASO) using Lipofectamine RNAiMax reagent (Invitrogen) according to manufacturer's instructions. Total RNA is extracted using RNeasy mini kit (Qiagen) 24 hrs post-transfection and cDNA is synthesized with ImProm-II reverse transcriptase (Promega). Total protein is extracted with RIPA buffer (Cell Signaling Technology) 48 hrs post transfection.

For another example, Huh7 cells are grown in DMEM with 10% FBS and 1×10⁵ cells are seeded in a 12-well plate and reverse transfected with 5, 20, or 80 nM ASO using Lipofectamine RNAiMAX (Invitrogen) according to manufacturer's instructions. For RT-PCR analysis, cells are treated with 50 μg/mL of CHX (Cell Signaling Technology) in DMSO for 3 hours 21 hours post transfection. Total RNA is extracted using RNeasy mini kit (Qiagen) 24 hrs post-transfection and cDNA is synthesized with ImProm-II reverse transcriptase (Promega). Total protein is extracted with RIPA buffer (Cell Signaling Technology) 48 hrs post transfection.

For another example, ReNcell VM cells are grown in complete NSC medium containing 20 ng/mL of bFGF and EGF each on laminin coated flasks (2D culture) until reaching ˜90% confluency. The cells are then detached by accutase treatment, washed with PBS, and cultured in complete NSC medium in ultra-low attachment surface 24-well polystyrene plate with 3, 8, 20 μM ASO for gymnotic (free) uptake. Total RNA is extracted using RNeasy mini kit (Qiagen) 72 hrs post-ASO addition to media and cDNA is synthesized with ImProm-II reverse transcriptase (Promega).

### Example 16: qPCR and RT-PCR Assays

For expression analysis of the productive mRNA, TaqMan qPCR (Thermo Fisher SC) is performed for PKD2. SYBR green qPCR or probe-based qPCR is performed for human PKD2 with forward primer and reverse primer, and probe. The cycling conditions are, for example, 30 sec at 95° C. for denaturation, 30 sec at 60° C. for annealing and 60 sec at 72° C. for extension for 30 cycles. For another example, the cycling conditions were 30 sec at 95° C. for denaturation, 30 sec at 55° C. for annealing, and 30 sec at 72° C. for extension for 29 cycles. For another example, The cycling conditions were 30 sec at 95° C. for denaturation, 30 sec at 55° C. for annealing, and 75 sec at 72° C. for extension for 28 cycles. For another example, The cycling conditions were 30 sec at 95° C. for denaturation, 30 sec at 56° C. for annealing, and 75 sec at 72° C. for extension for 28 cycles. The PCR products are separated on 5% polyacrylamide gel and quantified with Multi Gauge software Version 2.3.

### Example 17: Western Blotting

Protein extracts are quantified by colorimetric assay using Pierce BCA protein assay kit (ThermoFisher).

For example, immunoblotting is carried out with 25 μg of lysate. For another example, immunoblotting is carried out with 60 μg of lysate. For another example, immunoblotting is carried out with 120 μg of lysate. For another example, immunoblotting is carried out with 30 μg of lysate. The primary antibody and the secondary antibody are purchased. Blots are scanned using Typhoon RLA 9000 imager (General Electric). Densitometric analysis is carried out using Multi Gauge software Version 2.3.

### Example 18: Flow Cytometry

Cells are lifted from culture plates in FACS buffer. Cells are stained with APC-antibody (1:250). Data from 15,000 cells are collected on a Guava Easycyte 12HT (EMD Millipore) flow cytometer. Fluorescence minus one is used to determine the positive gate.

### Example 19: PKD2 Exon Region and 5′UTR Vectorized ASO Walk

To identify vectorized ASOs that can prevent the non-productive AS events, a systematic vectorized ASO walk can be performed in 5-nt or 2-nt steps along the AS event of interest. These vectorized ASOs can be expressed from a vector as a modified U1 snRNA or U7 snRNA, which contains an ASO sequence as its targeting sequence. FIG. 5 shows a systematic vectorized ASO walk along an NMD exon AS event of PKD2 pre-mRNA. FIGS. 6A and 6B show a systematic vectorized ASO walk along an alternative 5′ ss AS event of PKD2 pre-mRNA. FIG. 10 shows a systematic vectorized ASO walk along a 5′ UTR of PKD2 mRNA. The vectorized ASO is expressed as a modified U7 snRNA. RT-PCR analysis from transfected cell lines can identify several vectorized ASOs that lead to reduced AS in the PKD2 mRNA and increase in productive mRNA. The observed increase in PKD2 productive mRNA can be confirmed by TaqMan qPCR. The fold change of AS may be plotted vs the increase in productive mRNA (qPCR) to demonstrate that the vectorized ASOs are functioning on mechanism.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Exemplary Embodiments I

Described herein, in certain embodiments, is a method of modulating expression of a target protein in a cell having a pre-mRNA that is transcribed from a target gene and that comprises a non-sense mediated RNA decay-inducing exon (NMD exon), the method comprising contacting an agent or a vector encoding the agent to the cell, whereby the agent modulates splicing of the NMD exon from the pre-mRNA, thereby modulating the level of a processed mRNA that is processed from the pre-mRNA, and modulating the expression of the target protein in the cell, wherein the target protein is a polycystin 2 and the target gene is a PKD2 gene.

Described herein, in certain embodiments, is a method of treating or reducing the likelihood of developing a disease or condition in a subject in need thereof by modulating expression of a target protein in a cell of the subject, comprising: contacting the cell of the subject with an agent or a vector encoding the agent to the cell, whereby the agent modulates splicing of a non-sense mediated mRNA decay-inducing exon (NMD exon) from a pre-mRNA that is transcribed from a target gene and that comprises the NMD exon, thereby modulating the level of a processed mRNA that is processed from the pre-mRNA, and modulating the expression of the target protein in the cell of the subject, wherein the target protein is a polycystin 2 and the target gene is a PKD2 gene.

In some embodiments, the agent: (a) binds to a targeted portion of the pre-mRNA; (b) modulates binding of a factor involved in splicing of the NMD exon; or (c) a combination of (a) and (b).

In some embodiments, the agent interferes with binding of the factor involved in splicing of the NMD exon to a region of the targeted portion.

In some embodiments, the targeted portion of the pre-mRNA is proximal to the NMD exon.

In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of 5′ end of the NMD exon.

In some embodiments, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of 5′ end of the NMD exon.

In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of 3′ end of the NMD exon.

In some embodiments, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of 3′ end of the NMD exon.

In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site of GRCh38/hg38: chr4:88031085.

In some embodiments, the targeted portion of the pre-mRNA is about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site of GRCh38/hg38: chr4:88031085.

In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site of GRCh38/hg38: chr4:88031140.

In some embodiments, the targeted portion of the pre-mRNA is about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site of GRCh38/hg38: chr4:88031140.

In some embodiments, the targeted portion of the pre-mRNA is located in an intronic region between two canonical exonic regions of the pre-mRNA, and wherein the intronic region contains the NMD exon.

In some embodiments, the targeted portion of the pre-mRNA at least partially overlaps with the NMD exon.

In some embodiments, targeted portion of the pre-mRNA at least partially overlaps with an intron upstream or downstream of the NMD exon.

In some embodiments, the targeted portion of the pre-mRNA comprises 5′ NMD exon-intron junction or 3′ NMD exon-intron junction.

In some embodiments, the targeted portion of the pre-mRNA is within the NMD exon.

In some embodiments, the targeted portion of the pre-mRNA comprises about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon.

In some embodiments, the NMD exon comprises a sequence with at least 80%, at least 90%, or 100% sequence identity to a sequence selected from the group consisting of the sequences listed in Table 2. In some embodiments, the NMD exon comprises a sequence with at least 50%, 55%, 60%, 65%, 70%, 75%, 80%, 85%, 90%, 91%, 92%, 93%, 94%, 95%, 96%, 97%, 98%, 99%, 99.5%, 99.9%, or 100% sequence identity to a sequence selected from the group consisting of the sequences listed in Table 2.

In some embodiments, the NMD exon comprises a sequence selected from the group consisting of the sequences listed in Table 2.

In some embodiments, the pre-mRNA comprises a sequence with at least about 80%, 85%, 90%, 95%, 97%, or 100% sequence identity to a sequence selected from the group consisting of the sequences listed in Table 2 or Table 3.

In some embodiments, the pre-mRNA is encoded by a genetic sequence with at least about 80%, 85%, 90%, 95%, 97%, or 100% sequence identity to a sequence selected from the group consisting of the sequences listed in Table 2 or Table 3.

In some embodiments, the targeted portion of the pre-mRNA comprises a sequence with at least 80%, 85%, 90%, 95%, 97%, or 100% sequence identity to a region comprising at least 8 contiguous nucleic acids of a sequence selected from the group consisting of the sequences listed in Table 2 or Table 3.

In some embodiments, the agent is an antisense oligomer (ASO) and wherein the ASO comprises a sequence that is at least about 80%, 85%, 90%, 95%, 97%, or 100% complementary to at least 8 contiguous nucleic acids of a sequence selected from the group consisting of the sequences listed in Table 4.

In some embodiments, the targeted portion of the pre-mRNA is within the non-sense mediated RNA decay-inducing exon GRCh38/hg38: chr4:88031085 88031140.

In some embodiments, the targeted portion of the pre-mRNA is upstream or downstream of the non-sense mediated RNA decay-inducing exon GRCh38/hg38: chr4:88031085 88031140.

In some embodiments, the targeted portion of the pre-mRNA comprises an exon-intron junction of exon GRCh38/hg38: chr4:88031085 88031140.

In some embodiments, the polycystin 2 expressed from the processed mRNA is full-length polycystin 2 or wild-type polycystin 2.

In some embodiments, the polycystin 2 expressed from the processed mRNA is at least partially functional as compared to wild-type polycystin 2.

In some embodiments, the polycystin 2 expressed from the processed mRNA is at least partially functional as compared to full-length wild-type polycystin 2.

In some embodiments, the agent promotes exclusion of the NMD exon from the pre-mRNA.

In some embodiments, the exclusion of the NMD exon from the pre-mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to exclusion of the NMD exon from the pre-mRNA in a control cell.

In some embodiments, the agent increases the level of the processed mRNA in the cell.

In some embodiments, the level of the processed mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to a level of the processed mRNA in a control cell.

In some embodiments, the agent increases the expression of the target protein in the cell.

In some embodiments, a level of the target protein expressed from the pre-mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to a level of the target protein produced in a control cell.

In some embodiments, the disease or condition is induced by a loss-of-function mutation in the target protein.

In some embodiments, the disease or condition is associated with haploinsufficiency of a gene encoding the target protein, and wherein the subject has a first allele encoding a functional target protein, and a second allele from which the target protein is not produced or produced at a reduced level, or a second allele encoding a nonfunctional target protein or a partially functional target protein.

In some embodiments, the disease or condition is selected from the group consisting of: polycystic kidney disease with or without polycystic liver disease, autosomal dominant polycystic kidney disease, and intracranial aneurysm.

In some embodiments, the disease or condition is associated with an autosomal recessive mutation of a gene encoding the target protein, wherein the subject has a first allele encoding from which: (i) the target protein is not produced or produced at a reduced level compared to a wild-type allele; or (ii) the target protein produced is nonfunctional or partially functional compared to a wild-type allele, and a second allele from which: (iii) the target protein is produced at a reduced level compared to a wild-type allele and the target protein produced is at least partially functional compared to a wild-type allele; or (iv) the target protein produced is partially functional compared to a wild-type allele.

In some embodiments, the disease or condition is selected from the group consisting of: polycystic kidney disease with or without polycystic liver disease, autosomal dominant polycystic kidney disease, and intracranial aneurysm.

In some embodiments, the agent promotes exclusion of the NMD exon from the pre-mRNA and increases the expression of the target protein in the cell.

In some embodiments, the agent inhibits exclusion of the NMD exon from the pre-mRNA encoding the target protein.

In some embodiments, the exclusion of the NMD exon from the pre-mRNA in the cell contacted with the agent is decreased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to exclusion of the NMD exon from the pre-mRNA in a control cell.

In some embodiments, the agent decreases the level of the processed mRNA in the cell.

In some embodiments, the level of the processed mRNA in the cell contacted with the agent is decreased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to a level of the processed mRNA in a control cell.

In some embodiments, the agent decreases the expression of the target protein in the cell.

In some embodiments, a level of the target protein expressed from the pre-mRNA in the cell contacted with the agent is decreased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to a level of the target protein expressed in a control cell.

In some embodiments, the disease or condition is induced by a gain-of-function mutation in the target protein.

In some embodiments, the subject has an allele from which the target protein is produced at an increased level, or an allele encoding a mutant target protein that exhibits increased activity in the cell.

In some embodiments, the agent inhibits exclusion of the NMD exon from the pre-mRNA encoding the target protein and decreases the expression of the target protein in the cell.

In some embodiments, the agent is an antisense oligomer (ASO) and wherein the antisense oligomer comprises a backbone modification comprising a phosphorothioate linkage or a phosphorodiamidate linkage.

In some embodiments, the agent is an antisense oligomer (ASO) and wherein the antisense oligomer comprises a phosphorodiamidate morpholino, a locked nucleic acid, a peptide nucleic acid, a 2′-O-methyl, a 2′-Fluoro, or a 2′-O-methoxyethyl moiety.

In some embodiments, the agent is an antisense oligomer (ASO) and wherein the antisense oligomer comprises at least one modified sugar moiety.

In some embodiments, each sugar moiety is a modified sugar moiety.

In some embodiments, the agent is an antisense oligomer (ASO) and wherein the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

In some embodiments, the agent is an antisense oligomer (ASO) and wherein the antisense oligomer is at least 80%, at least 85%, at least 90%, at least 95%, at least 98%, at least 99%, or 100%, complementary to the targeted portion of the pre-mRNA.

In some embodiments, the method further comprises assessing mRNA level or expression level of the target protein.

In some embodiments, the subject is a human.

In some embodiments, the subject is a non-human animal.

In some embodiments, the subject is a fetus, an embryo, or a child.

In some embodiments, the cells are ex vivo.

In some embodiments, the agent is administered by intrathecal injection, intracerebroventricular injection, intraperitoneal injection, intramuscular injection, subcutaneous injection, intravitreal, or intravenous injection of the subject.

In some embodiments, the method further comprises administering a second therapeutic agent to the subject.

In some embodiments, the second therapeutic agent is a small molecule.

In some embodiments, the second therapeutic agent is an antisense oligomer.

In some embodiments, the second therapeutic agent corrects intron retention.

In some embodiments, the method treats the disease or condition.

Described herein, in certain embodiments, is a composition comprising a non-sense mediated RNA decay exon (NSE)-modulating agent that interacts with a target motif within a pre-mRNA to modulate exclusion of an NSE from a processed mRNA transcript and to modulate inclusion of a canonical exon in the processed mRNA transcript, wherein the target motif is located: (i) in an intronic region between two canonical exons, (ii) in one of the two canonical exons, or (iii) in a region spanning both an intron and canonical exon; wherein the NSE comprises: (a) only a portion of a canonical exon, or (b) a canonical exon and at least a portion of an intron adjacent to the canonical exon; and wherein the NSE-modulating agent modulates exclusion of an NSE from the pre-mRNA transcript and modulates inclusion of a canonical exon in the processed mRNA transcript.

In some embodiments, the NSE-modulating agent promotes exclusion of an NSE from the pre-mRNA transcript and promotes inclusion of a canonical exon in the processed mRNA transcript.

In some embodiments, the processed mRNA transcript encodes a target protein and the NSE-modulating agent increases expression of a target protein in a cell containing the pre-mRNA.

In some embodiments, the target protein is polycystin 2.

Described herein, in certain embodiments, is a composition comprising a non-sense mediated RNA decay alternative 5′ or 3′ splice site (NSASS)-modulating agent that interacts with a target motif within a pre-mRNA to modulate splicing at an alternative 5′ or 3′ splice site of a pre-mRNA and to modulate inclusion of a canonical exon in a processed mRNA transcript that is processed from the pre-mRNA, wherein the target motif is located: (i) in an intronic region between two canonical exons, (ii) in one of the two canonical exons, or (iii) in a region spanning both an intron and canonical exon; wherein modulating splicing at the alternative 5′ or 3′ splice site of the pre-mRNA modulates exclusion of an exon from the pre-mRNA transcript, wherein the exon comprises: (a) only a portion of a canonical exon, or (b) a canonical exon and at least a portion of an intron adjacent to the canonical exon; and wherein the NSASS-modulating agent modulates exclusion of the exon from the pre-mRNA transcript and modulates inclusion of a canonical exon in the processed mRNA transcript.

In some embodiments, the NSASS-modulating agent promotes exclusion of the exon resulting from splicing at the alternative 5′ or 3′ splice site from the pre-mRNA transcript and promotes inclusion of a canonical exon in the processed mRNA transcript.

In some embodiments, the processed mRNA transcript encodes a target protein and the NSASS-modulating agent increases expression of a target protein in a cell containing the pre-mRNA.

In some embodiments, the target protein is selected from the group consisting of, PKD2.

Described herein, in certain embodiments, is a composition comprising a non-sense mediated RNA decay exon (NSE)-modulating agent that modulates expression of a target protein in a cell comprising a pre-mRNA that encodes the target protein, wherein the pre-mRNA comprises: an alternative nonsense mediated RNA decay-inducing (NMD) exon comprising an alternative 5′ splice site upstream of the canonical 5′ splice site in reference to an intron following a canonical exon and within the canonical exon, or downstream of the canonical 5′ splice site in reference to an intron following the canonical exon and within the intron; wherein the NSE-modulating agent modulates processing of an mRNA transcript from the pre-mRNA by modulating splicing of the pre-mRNA at the alternative 5′ splice site, wherein the splicing of the pre-mRNA at the alternative 5′ splice site modulates the expression of the target protein in a cell.

In some embodiments, the target protein is PKD2.

In some embodiments, the splicing of the pre-mRNA at the alternative 5′ splice site increases the expression of the target protein in a cell.

In some embodiments, the alternative nonsense mediated RNA decay-inducing (NMD) exon comprises an alternative 5′ splice site upstream of the canonical 5′ splice site in reference to an intron following a canonical exon and within the canonical exon.

In some embodiments, the alternative nonsense mediated RNA decay-inducing (NMD) exon comprises an alternative 5′ splice site downstream of the canonical 5′ splice site in reference to an intron following the canonical exon and within the intron.

Described herein, in certain embodiments, is a composition comprising a non-sense mediated RNA decay alternative 5′ or 3′ splice site (NSASS)-modulating agent that modulates expression of a target protein in a cell comprising a pre-mRNA that encodes the target protein, wherein the pre-mRNA comprises: an exon comprising an alternative 5′ splice site upstream of the canonical 5′ splice site in reference to an intron following a canonical exon and within the canonical exon, or downstream of the canonical 5′ splice site in reference to an intron following the canonical exon and within the intron; wherein the NSASS-modulating agent modulates processing of an mRNA transcript from the pre-mRNA by modulating splicing of the pre-mRNA at the alternative 5′ splice site, wherein the splicing of the pre-mRNA at the alternative 5′ splice site modulates the expression of the target protein in a cell.

In some embodiments, the target protein is polycystin 2.

In some embodiments, the splicing of the pre-mRNA at the alternative 5′ splice site increases the expression of the target protein in a cell.

In some embodiments, the pre-mRNA comprises an exon comprising an alternative 5′ splice site upstream of the canonical 5′ splice site in reference to an intron following a canonical exon and within the canonical exon.

In some embodiments, the pre-mRNA comprises an exon comprising an alternative 5′ splice site downstream of the canonical 5′ splice site in reference to an intron following the canonical exon and within the intron.

Described herein, in certain embodiments, is a composition comprising a non-sense mediated RNA decay exon (NSE)-modulating agent that modulates expression of a target protein in a cell comprising a pre-mRNA that encodes the target protein, wherein the pre-mRNA comprises an alternative nonsense mediated RNA decay-inducing (NMD) exon comprising an alternative 3′ splice site downstream of the canonical 3′ splice site in reference to an intron preceding a canonical exon and within the canonical exon, or upstream of the canonical 3′ splice site in reference to an intron preceding the canonical exon and within the intron, wherein the NSE-modulating agent modulates processing of an mRNA transcript from the pre-mRNA by modulating splicing of the pre-mRNA at the alternative 3′ splice site, and wherein the splicing of the pre-mRNA at the alternative 3′ splice site modulates the expression of the target protein in a cell.

In some embodiments, the target protein is polycystin 2.

In some embodiments, the splicing of the pre-mRNA at the alternative 3′ splice site increases the expression of the target protein in a cell.

In some embodiments, the alternative nonsense mediated RNA decay-inducing (NMD) exon comprises an alternative 3′ splice site downstream of the canonical 3′ splice site in reference to an intron preceding a canonical exon and within the canonical exon.

In some embodiments, the alternative nonsense mediated RNA decay-inducing (NMD) exon comprises an alternative 3′ splice site upstream of the canonical 3′ splice site in reference to an intron preceding the canonical exon and within the intron.

Described herein, in certain embodiments, is a composition comprising a non-sense mediated RNA decay alternative 5′ or 3′ splice site (NSASS)-modulating agent that modulates expression of a target protein in a cell comprising a pre-mRNA that encodes the target protein, wherein the pre-mRNA comprises an exon comprising an alternative 3′ splice site downstream of the canonical 3′ splice site in reference to an intron preceding a canonical exon and within the canonical exon, or upstream of the canonical 3′ splice site in reference to an intron preceding the canonical exon and within the intron, wherein the NSASS-modulating agent modulates processing of an mRNA transcript from the pre-mRNA by modulating splicing of the pre-mRNA at the alternative 3′ splice site, and wherein the splicing of the pre-mRNA at the alternative 3′ splice site modulates the expression of the target protein in a cell.

In some embodiments, the target protein is polycystin 2.

In some embodiments, the splicing of the pre-mRNA at the alternative 3′ splice site increases the expression of the target protein in a cell.

In some embodiments, the pre-mRNA comprises an exon comprising an alternative 3′ splice site downstream of the canonical 3′ splice site in reference to an intron preceding a canonical exon and within the canonical exon.

In some embodiments, the pre-mRNA comprises an exon comprising an alternative 3′ splice site upstream of the canonical 3′ splice site in reference to an intron preceding the canonical exon and within the intron.

In some embodiments, the agent is a small molecule.

In some embodiments, the agent is a polypeptide.

In some embodiments, the polypeptide is a nucleic acid binding protein.

In some embodiments, the nucleic acid binding protein contains a TAL-effector or zinc finger binding domain.

In some embodiments, the nucleic acid binding protein is a Cas family protein.

In some embodiments, the polypeptide is accompanied by or complexed with one or more nucleic acid molecules.

In some embodiments, the agent is an antisense oligomer (ASO) complementary to the targeted region of the pre-mRNA.

In some embodiments, the agent is at least 75%, at least 80%, at least 85%, at least 90%, at least 95%, at least 98%, at least 99%, or 100%, complementary to the targeted region of the pre-mRNA encoding the target protein.

In some embodiments, the agent comprises a backbone modification comprising a phosphorothioate linkage or a phosphorodiamidate linkage.

In some embodiments, the agent comprises a phosphorodiamidate morpholino.

In some embodiments, the agent comprises a locked nucleic acid.

In some embodiments, the agent comprises a peptide nucleic acid.

In some embodiments, the agent comprises a 2′-O-methyl.

In some embodiments, the agent comprises a 2′-Fluoro, or a 2′-O-methoxyethyl moiety.

In some embodiments, the agent comprises at least one modified sugar moiety.

In some embodiments, each sugar moiety is a modified sugar moiety.

In some embodiments, the agent is an antisense oligomer, and wherein the agent consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

Described herein, in certain embodiments, is a composition comprising a nucleic acid molecule that encodes for the agent according to the composition as provided herein.

In some embodiments, the nucleic acid molecule is incorporated into a viral delivery system.

In some embodiments, the viral delivery system is an adenovirus-associated vector.

Described herein, in certain embodiments, is a method of modulating protein expression, comprising: contacting a non-sense mediated RNA decay exon (NSE)-modulating agent to a target motif within a pre-mRNA, wherein the NSE comprises (i) only a portion of a canonical exon, or (ii) a canonical exon and at least a portion of an intron adjacent to the canonical exon; wherein the pre-mRNA is processed to form a processed mRNA transcript, wherein the NSE-modulating agent modulates exclusion of an NSE from the pre-mRNA transcript and modulates inclusion of the canonical exon in the processed mRNA transcript; and wherein the processed mRNA transcript is translated, wherein the exclusion of the NSE and inclusion of the canonical exon modulates target protein expression relative to the target protein expression of an equivalent mRNA transcript comprising the NSE instead of the canonical exon.

In some embodiments, the target motif is located in an intronic region between two canonical exons.

In some embodiments, the target motif is located in one of the two canonical exons.

In some embodiments, the target motif is located in a region spanning both an intron and a canonical exon.

In some embodiments, the target protein is polycystin 2.

Described herein, in certain embodiments, is a method of modulating expression of a target protein by a cell having a pre-mRNA that encodes the target protein, wherein the pre-mRNA comprises: an alternative nonsense mediated RNA decay-inducing (NMD) exon comprising an alternative 3′ splice site downstream of the canonical 3′ splice site in reference to an intron preceding a canonical exon and within the canonical exon, or upstream of the 3′ splice site in reference to an intron preceding the canonical exon and within the intron, the method comprising contacting a non-sense mediated RNA decay exon (NSE)-modulating agent to the cell, wherein the non-sense mediated RNA decay exon (NSE)-modulating agent t modulates processing of an mRNA transcript from the pre-mRNA by modulating splicing of the pre-mRNA at the alternative 3′ splice site, and wherein the splicing of the pre-mRNA at the alternative 3′ splice site modulates the expression of the target protein.

In some embodiments, the target protein is polycystin 2.

Described herein, in certain embodiments, is a method of modulating expression of a target protein by a cell having a pre-mRNA that encodes the target protein, wherein the pre-mRNA comprises: an alternative nonsense mediated RNA decay-inducing (NMD) exon comprising an alternative 5′ splice site upstream of the canonical 5′ splice site in reference to an intron following a canonical exon and within the canonical exon, or downstream of the canonical 5′ splice site in reference to an intron following the canonical exon and within the intron, the method comprising contacting a non-sense mediated RNA decay exon (NSE)-modulating agent to the cell, wherein the non-sense mediated RNA decay exon (NSE)-modulating agent modulates processing of an mRNA transcript from the pre-mRNA by modulating splicing of the pre-mRNA at the alternative 5′ splice site, and wherein the splicing of the pre-mRNA at the alternative 5′ splice site modulates the expression of the target protein.

In some embodiments, the target protein is polycystin 2.

In some embodiments, the non-sense mediated RNA decay exon (NSE)-modulating agent binds to a targeted portion of the pre-mRNA.

In some embodiments, the wherein the non-sense mediated RNA decay exon (NSE)-modulating agent binds to a factor involved in splicing of the NSE or NMD exon.

In some embodiments, the wherein the non-sense mediated RNA decay exon (NSE)-modulating agent inhibits activity of a factor involved in splicing of the NMD exon.

In some embodiments, the non-sense mediated RNA decay exon (NSE)-modulating agent interferes with binding of a factor involved in splicing of the NMD exon to a region of the targeted portion of the pre-mRNA.

In some embodiments, modulation of splicing of the pre-mRNA increases the expression of the target protein.

In some embodiments, the level the target protein in the cell is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell.

In some embodiments, modulation of splicing of the pre-mRNA in-creases production of the processed mRNA encoding the target protein.

In some embodiments, the level of processed mRNA encoding the target protein in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell.

In some embodiments, the target protein is the canonical isoform of the protein.

In some embodiments, the target protein is polycystin 2.

In some embodiments, the non-sense mediated RNA decay exon (NSE)-modulating agent is the composition as provided herein.

Described herein, in certain embodiments, is a pharmaceutical composition comprising a therapeutic agent comprising the composition as provided herein; and a pharmaceutically acceptable excipient and/or a delivery vehicle.

Described herein, in certain embodiments, is a method of treating or reducing the likelihood of developing a disease or condition in a subject in need thereof, the method comprising: administering to the subject a pharmaceutical composition as provided herein to a subject in need thereof.

Described herein, in certain embodiments, is a method of treating or reducing the likelihood of developing a disease or condition in a subject in need thereof, the method comprising: administering to the subject a pharmaceutical composition comprising: (a) a non-sense mediated RNA decay exon (NSE)-modulating agent that interacts with a target motif within a pre-mRNA to modulate exclusion of an NSE from a processed mRNA transcript and to modulate inclusion of a canonical exon in the processed mRNA transcript,
wherein the NSE comprises (i) only a portion of a canonical exon, or (ii) a canonical exon and at least a portion of an intron adjacent to the canonical exon; and (b) a pharmaceutically acceptable excipient and/or a delivery vehicle, wherein the disease or condition is treated or prevented in the subject by the administration of the NSE-modulating agent by a modulation in expression of a target protein translated from the processed mRNA transcript.

In some embodiments, the target protein is polycystin 2.

Described herein, in certain embodiments, is a method of treating or reducing the likelihood of developing a disease or condition in a subject in need thereof by modulating expression of a target protein in a cell of the subject, wherein the cell of the subject has a pre-mRNA that encodes the target protein, wherein the pre-mRNA comprises: (a) a canonical exon preceded by a canonical intron flanking a 5′ end of the canonical exon; and (b) an alternative nonsense mediated RNA decay-inducing (NMD) exon comprising an alternative 3′ splice site downstream of the canonical 3′ splice site in reference to the intron preceding the canonical exon and within the canonical exon, or upstream of the canonical 3′ splice site in reference to the intron preceding the canonical exon and within the canonical intron, the method comprising contacting a therapeutic agent to the cell, wherein the therapeutic agent modulates processing of an mRNA transcript from the pre-mRNA by modulating splicing of the pre-mRNA at the alternative 3′ splice site, and wherein the splicing of the pre-mRNA at the alternative 3′ splice site modulates the expression of the target protein in the cell of the subject.

In some embodiments, the target protein is polycystin 2.

Described herein, in certain embodiments, is a method of treating or reducing the likelihood of developing a disease or condition in a subject in need thereof by modulating expression of a target protein in a cell of the subject, wherein the cell of the subject has a pre-mRNA that encodes the target protein, wherein the pre-mRNA comprises: (a) a canonical exon followed by a canonical intron flanking a 3′ end of the canonical exon; and (b) an alternative nonsense mediated RNA decay-inducing (NMD) exon comprising an alternative 5′ splice site upstream of the canonical 5′ splice site in reference to the intron following the canonical exon and within the canonical exon, or downstream of the canonical 5′ splice site in reference to the intron following the canonical exon and within the canonical intron, the method comprising contacting a therapeutic agent to the cell, wherein the therapeutic agent modulates processing of an mRNA transcript from the pre-mRNA by modulating splicing of the pre-mRNA at the alternative 5′ splice site, and wherein the splicing of the pre-mRNA at the alternative 5′ splice site modulates the expression of the target protein in the cell of the subject.

In some embodiments, the target protein is polycystin 2.

In some embodiments, the disease is polycystic kidney disease with or without polycystic liver disease, autosomal dominant polycystic kidney disease, or intracranial aneurysm.

In some embodiments, the disease or the condition is caused by a deficient amount or activity of the target protein.

In some embodiments, the therapeutic agent increases the level of the processed mRNA encoding the target protein in the cell.

In some embodiments, the therapeutic agent increases the expression of the target protein in the cell.

In some embodiments, the level of processed mRNA encoding the target protein in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell.

In some embodiments, the level the target protein in the cell is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell.

In some embodiments, the method further comprises assessing mRNA levels or expression levels of the target protein.

In some embodiments, the method further comprises assessing the subject's genome for at least one genetic mutation associated with the disease.

In some embodiments, at least one genetic mutation is within a locus of a gene associated with the disease.

In some embodiments, at least one genetic mutation is within a locus associated with expression of a gene associated with the disease.

In some embodiments, at least one genetic mutation is within the PKD2 gene locus.

In some embodiments, at least one genetic mutation is within a locus associated with PKD2 gene expression.

In some embodiments, the subject is a human.

In some embodiments, the subject is a non-human animal.

In some embodiments, the subject is a fetus, an embryo, or a child.

In some embodiments, the cell or the cells is ex vivo, or in a tissue, or organ ex vivo.

In some embodiments, the therapeutic agent is administered to the subject by intracerebroventricular injection, intraperitoneal injection, intramuscular injection, intrathecal injection, subcutaneous injection, oral administration, synovial injection, intravitreal administration, subretinal injection, topical application, implantation, or intravenous injection.

In some embodiments, the method treats the disease or condition.

Described herein, in certain embodiments, is a therapeutic agent for use in the method as provided herein.

Described herein, in certain embodiments, is a pharmaceutical composition comprising the therapeutic agent of as provided herein, and a pharmaceutically acceptable excipient.

Described herein, in certain embodiments, is a method of treating or reducing the likelihood of developing a disease or condition in a subject in need thereof, comprising administering the pharmaceutical composition as provided herein by intracerebroventricular injection, intraperitoneal injection, intramuscular injection, intrathecal injection, subcutaneous injection, oral administration, synovial injection, intravitreal administration, subretinal injection, topical application, implantation, or intravenous injection to the subject.

In some embodiments, the method treats the subject.

Described herein, in certain embodiments, is a composition comprising a non-sense mediated RNA decay exon (NSE)-modulating agent or a viral vector encoding the agent that interacts with a target motif within a pre-mRNA that is transcribed from a target gene to modulate exclusion of an NSAE from a processed mRNA transcript and to modulate inclusion of a canonical exon in the processed mRNA transcript, wherein the target motif is located: (i) in an intronic region between two canonical exons, (ii) in one of the two canonical exons, or (iii) in a region spanning both an intron and canonical exon; wherein the NSAE comprises: (a) only a portion of a canonical exon, or (b) a canonical exon and at least a portion of an intron adjacent to the canonical exon; wherein the NSAE-modulating agent modulates exclusion of an NSAE from the processed mRNA transcript and modulates inclusion of a canonical exon in the processed mRNA transcript; and wherein the target gene is a PKD2 gene.

In some embodiments, the NSAE-modulating agent promotes exclusion of an NSAE from the processed mRNA transcript and promotes inclusion of a canonical exon in the processed mRNA transcript.

In some embodiments, the processed mRNA transcript encodes a target protein and the NSAE-modulating agent increases expression of the target protein in a cell containing the pre-mRNA, and wherein the target protein is PKD2.

### Exemplary Embodiments II

Described herein, in certain embodiments, is a method of modulating expression of a target protein in a cell having a pre-mRNA that is transcribed from a target gene and that comprises a non-sense mediated RNA decay-inducing exon (NMD exon), the method comprising: contacting an agent or a vector encoding the agent to the cell, whereby the agent modulates splicing of the NMD exon from the pre-mRNA, thereby modulating the level of a processed mRNA that is processed from the pre-mRNA, and modulating the expression of the target protein in the cell, wherein the target protein is encoded by a PKD2 gene.

Described herein, in certain embodiments, is a method of treating or reducing the likelihood of developing a disease or condition in a subject in need thereof by modulating expression of a target protein in a cell of the subject, comprising: contacting an agent or a vector encoding the agent to the cell of the subject, whereby the agent modulates splicing of a non-sense mediated mRNA decay-inducing exon (NMD exon) from a pre-mRNA that is transcribed from a target gene and that comprises the NMD exon, thereby modulating the level of a processed mRNA that is processed from the pre-mRNA, and modulating the expression of the target protein in the cell of the subject, wherein the target protein encoded by a PKD2 gene.

In some embodiments, the target protein is polycystin 2. In some embodiments, the disease or condition is a disease or condition associated with a deficiency in amount or activity of polycystin 2. In some embodiments, the disease or condition is a disease or condition associated with a deficiency in amount or activity of polycystin 1. In some embodiments, the disease or condition is a disease or condition associated with a deficiency in amount or activity of a protein that polycystin 2 functionally augments, compensates for, replaces, or functionally interacts with.

In some embodiments, the agent: (a) binds to a targeted portion of the pre-mRNA; (b) modulates binding of a factor involved in splicing of the NMD exon; or (c) a combination of (a) and (b). In some embodiments, the agent interferes with binding of the factor involved in splicing of the NMD exon.

In some embodiments, the targeted portion of the pre-mRNA is proximal to the NMD exon. In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of the 5′ end of the NMD exon. In some embodiments, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of the 5′ end of the NMD exon. In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of the 3′ end of the NMD exon. In some embodiments, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of the 3′ end of the NMD exon.

In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site of GRCh38/hg38: chr4:88031085. In some embodiments, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site of GRCh38/hg38: chr4:88031085. In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site of GRCh38/hg38: chr4:88031140. In some embodiments, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site of GRCh38/hg38: chr4:88031140.

In some embodiments, the targeted portion of the pre-mRNA is located in an intronic region between two canonical exonic regions of the pre-mRNA, and wherein the intronic region contains the NMD exon. In some embodiments, the targeted portion of the pre-mRNA at least partially overlaps with the NMD exon. In some embodiments, the targeted portion of the pre-mRNA at least partially overlaps with an intron upstream or downstream of the NMD exon. In some embodiments, the targeted portion of the pre-mRNA comprises 5′ NMD exon-intron junction or 3′ NMD exon-intron junction. In some embodiments, the targeted portion of the pre-mRNA is within the NMD exon. In some embodiments, the targeted portion of the pre-mRNA comprises about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon.

In some embodiments, the NMD exon comprises a sequence with at least 80%, at least 90%, or 100% sequence identity to a sequence selected from the group consisting of the sequences listed in Table 2. In some embodiments, the NMD exon comprises a sequence selected from the group consisting of the sequences listed in Table 2. In some embodiments, the pre-mRNA comprises a sequence with at least about 80%, 85%, 90%, 95%, 97%, or 100% sequence identity to a sequence selected from the group consisting of the sequences listed in Table 2 or Table 3. In some embodiments, the pre-mRNA is encoded by a genetic sequence with at least about 80%, 85%, 90%, 95%, 97%, or 100% sequence identity to a sequence selected from the group consisting of the sequences listed in Table 2 or Table 3. In some embodiments, the targeted portion of the pre-mRNA comprises a sequence with at least 80%, 85%, 90%, 95%, 97%, or 100% sequence identity to a region comprising at least 8 contiguous nucleic acids of a sequence selected from the group consisting of the sequences listed in Table 2 or Table 3. In some embodiments, the agent is an antisense oligomer (ASO) and wherein the ASO comprises a sequence that is at least about 80%, 85%, 90%, 95%, 97%, or 100% sequence identity to at least 8, 9, 10, 11, 12, 13, 14, 15, 16, 17 or 18 contiguous nucleic acids of a sequence selected from the group consisting of the sequences listed in Table 4. In some embodiments, the targeted portion of the pre-mRNA is within the non-sense mediated RNA decay-inducing exon GRCh38/hg38: chr4:88031085-88031140. In some embodiments, the targeted portion of the pre-mRNA is upstream or downstream of the non-sense mediated RNA decay-inducing exon GRCh38/hg38: chr4:88031085-88031140. In some embodiments, the targeted portion of the pre-mRNA comprises an exon-intron junction of the non-sense mediated RNA decay-inducing exon GRCh38/hg38: chr4:88031085 88031140.

In some embodiments, the polycystin 2 expressed from the processed mRNA is full-length polycystin 2 or wild-type polycystin 2. In some embodiments, the polycystin 2 expressed from the processed mRNA is at least partially functional as compared to wild-type polycystin 2. In some embodiments, the polycystin 2 expressed from the processed mRNA is at least partially functional as compared to full-length wild-type polycystin 2.

In some embodiments, the agent modulates splicing of the NMD exon from the pre-mRNA and promotes exclusion of the NMD exon from the pre-mRNA, thereby modulating the level of a processed mRNA that is processed from the pre-mRNA and that lacks the NMD exon. In some embodiments, the exclusion of the NMD exon from the pre-mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to exclusion of the NMD exon from the pre-mRNA in a control cell. In some embodiments, the method results in an increase in the level of the processed mRNA in the cell. In some embodiments, the level of the processed mRNA in the cell contacted with the agent is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to a level of the processed mRNA in a control cell. In some embodiments, the agent increases the expression of the target protein in the cell. In some embodiments, a level of the target protein is increased by about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to a level of the target protein produced in a control cell.

In some embodiments, the NMD exon comprises a premature termination codon (PTC). In some embodiments, the disease or condition is associated with a loss-of-function mutation in the target gene or the target protein. In some embodiments, the disease or condition is associated with haploinsufficiency of the target gene, and wherein the subject has a first allele encoding functional polycystin 2, and a second allele from which polycystin 2 is not produced or produced at a reduced level, or a second allele encoding nonfunctional polycystin 2 or partially functional polycystin 2. In some embodiments, one or both alleles are hypomorphs or partially functional. In some embodiments, the disease or condition is selected from the group consisting of: polycystic kidney disease with or without polycystic liver disease, autosomal dominant polycystic kidney disease, and intracranial aneurysm. In some embodiments, the disease or condition is associated with a mutation of a PKD1 or PKD2 gene, wherein the subject has a first allele encoding from which: (i) the target protein is not produced or produced at a reduced level compared to a wild-type allele; or (ii) the target protein produced is nonfunctional or partially functional compared to a wild-type allele, and a second allele from which: (iii) the target protein is produced at a reduced level compared to a wild-type allele and the target protein produced is at least partially functional compared to a wild-type allele; or (iv) the target protein produced is partially functional compared to a wild-type allele.

In some embodiments, the disease or condition is selected from the group consisting of: polycystic kidney disease with or without polycystic liver disease, autosomal dominant polycystic kidney disease, and intracranial aneurysm. In some embodiments, the mutation is a hypomorphic mutation. In some embodiments, the disease or condition is associated with a mutation of a PKD2 gene. In some embodiments, the disease or condition is associated with a mutation of a PKD1 gene. In some embodiments, the mutation in PKD1 comprises a mutation in a region of polycystin 1 that interacts with polycystin 2. In some embodiments, the mutation in PKD1 comprises a mutation that interferes the interaction between Polycystin 1 and Polycystin 2. In some embodiments, the mutation in PKD1 comprises a mutation that weakens the interaction between Polycystin 1 and Polycystin 2. In some embodiments, the mutation in PKD1 comprises a mutation that reduces the interaction between Polycystin 1 and Polycystin 2. In some embodiments, the mutation in PKD1 comprises a mutation that blocks the interaction between Polycystin 1 and Polycystin 2. In some embodiments, the mutation in PKD1 is a mutation in a region of polycystin 1 that interacts with polycystin 2. In some embodiments, the mutation in PKD1 is a mutation that interferes the interaction between Polycystin 1 and Polycystin 2. In some embodiments, the mutation in PKD1 is a mutation that weakens the interaction between Polycystin 1 and Polycystin 2. In some embodiments, the mutation in PKD1 is a mutation that reduces the interaction between Polycystin 1 and Polycystin 2. In some embodiments, the mutation in PKD1 is a mutation that blocks the interaction between Polycystin 1 and Polycystin 2.

In some embodiments, the agent promotes exclusion of the NMD exon from the pre-mRNA, thereby modulating the level of a processed mRNA that is processed from the pre-mRNA and that lacks the NMD exon and increases the expression of the target protein in the cell. In some embodiments, the agent is an antisense oligomer (ASO) and wherein the antisense oligomer comprises a backbone modification comprising a phosphorothioate linkage or a phosphorodiamidate linkage. In some embodiments, the agent is an antisense oligomer (ASO) and wherein the antisense oligomer comprises a phosphorodiamidate morpholino, a locked nucleic acid, a peptide nucleic acid, a 2′-O-methyl, a 2′-Fluoro, or a 2′-O-methoxyethyl moiety. In some embodiments, the agent is an antisense oligomer (ASO) and wherein the antisense oligomer comprises at least one modified sugar moiety. In some embodiments, each sugar moiety is a modified sugar moiety. In some embodiments, the agent is an antisense oligomer (ASO) and wherein the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases. In some embodiments, the agent is an antisense oligomer (ASO) and wherein the antisense oligomer is at least 80%, at least 85%, at least 90%, at least 95%, at least 98%, at least 99%, or 100%, complementary to the targeted portion of the pre-mRNA.

In some embodiments, the method further comprises assessing processed mRNA level or expression level of the target protein. In some embodiments, the subject is a human. In some embodiments, the subject is a non-human animal. In some embodiments, the subject is a fetus, an embryo, or a child. In some embodiments, the cells are ex vivo. In some embodiments, the agent is administered by intrathecal injection, intracerebroventricular injection, intraperitoneal injection, intramuscular injection, subcutaneous injection, intravitreal, or intravenous injection of the subject. In some embodiments, the method further comprises administering a second therapeutic agent to the subject. In some embodiments, the second therapeutic agent is a small molecule. In some embodiments, the second therapeutic agent is an antisense oligomer. In some embodiments, the second therapeutic agent corrects intron retention. In some embodiments, the method treats the disease or condition.

Described herein, in certain embodiments, is a composition comprising an agent or a vector encoding the agent that modulates splicing of a non-sense mediated RNA decay-inducing exon (NMD exon) from a pre-mRNA that is transcribed from a target gene and that comprises the NMD exon, thereby modulating the level of a processed mRNA that is processed from the pre-mRNA, and modulating expression of a target protein in a cell having the pre-mRNA, wherein the target protein is encoded by a PKD2 gene. Described herein, in certain embodiments, is a composition comprising an agent or a vector encoding the agent that modulates splicing of a non-sense mediated mRNA decay-inducing exon (NMD exon) from a pre-mRNA that is transcribed from a target gene and that comprises the NMD exon, thereby treating a disease or condition in a subject in need thereof by modulating the level of a processed mRNA that is processed from the pre-mRNA, and modulating expression of a target protein in a cell of the subject, wherein the target protein is encoded by a PKD2 gene. Described herein, in certain embodiments, is a pharmaceutical composition comprising the composition as described herein; and a pharmaceutically acceptable excipient and/or a delivery vehicle. Described herein, in certain embodiments, is a composition comprising a non-sense mediated RNA decay alternative splice site (NSASS) modulating agent or a viral vector encoding the agent, wherein the agent modulates expression of a target protein in a cell comprising a pre-mRNA that is transcribed from a target gene and encodes the target protein, wherein the pre-mRNA comprises an alternative 5′ splice-site downstream of a canonical 5′ splice-site, wherein a processed mRNA that is produced by splicing of the pre-mRNA at the alternative 5′ splice-site undergoes non-sense mediated RNA decay, wherein the agent modulates processing of the pre-mRNA by modulating splicing at the alternative 5′ splice-site; and wherein the target gene is PKD2.

In some embodiments, the agent modulates processing of the pre-mRNA by preventing or decreasing splicing at the alternative 5′ splice-site. In some embodiments, the agent modulates processing of the pre-mRNA by promoting or increasing splicing at the canonical 5′ splice-site. In some embodiments, modulating the splicing of the pre-mRNA at the alternative 5′ splice-site increases the expression of the target protein in the cell. In some embodiments, the processed mRNA that is produced by splicing of the pre-mRNA at the alternative 5′ splice-site comprises a premature termination codon (PTC).

In some embodiments, the agent is a small molecule. In some embodiments, the agent is a polypeptide. In some embodiments, the polypeptide is a nucleic acid binding protein. In some embodiments, the nucleic acid binding protein contains a TAL-effector or zinc finger binding domain. In some embodiments, the nucleic acid binding protein is a Cas family protein. In some embodiments, the polypeptide is accompanied by or complexed with one or more nucleic acid molecules. In some embodiments, the agent is an antisense oligomer (ASO) complementary to the targeted region of the pre-mRNA. In some embodiments, the agent is at least 75%, at least 80%, at least 85%, at least 90%, at least 95%, at least 98%, at least 99%, or 100%, complementary to the targeted region of the pre-mRNA encoding the target protein. In some embodiments, the agent comprises a backbone modification comprising a phosphorothioate linkage or a phosphorodiamidate linkage. In some embodiments, the agent comprises a phosphorodiamidate morpholino. In some embodiments, the agent comprises a locked nucleic acid. In some embodiments, the agent comprises a peptide nucleic acid. In some embodiments, the agent comprises a 2′-O-methyl. In some embodiments, the agent comprises a 2′-Fluoro, or a 2′-O-methoxyethyl moiety. In some embodiments, the agent comprises at least one modified sugar moiety. In some embodiments, each sugar moiety is a modified sugar moiety. In some embodiments, the agent is an antisense oligomer, and wherein the agent consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases.

Described herein, in certain embodiments, is a composition comprising a nucleic acid molecule that encodes for the agent according to the composition as described herein. In some embodiments, the nucleic acid molecule is incorporated into a viral delivery system. In some embodiments, the viral delivery system is an adenovirus-associated vector. In some embodiments, the viral vector is an adenovirus-associated viral vector.

Described herein, in certain embodiments, is a method of modulating expression of a target protein in a cell comprising a pre-mRNA that is transcribed from a target gene and encodes the target protein, the method comprising: contacting a non-sense mediated RNA decay alternative splice site (NSASS) modulating agent or a viral vector encoding the agent to the cell, wherein the pre-mRNA comprises an alternative 5′ splice-site downstream of a canonical 5′ splice-site, wherein a processed mRNA that is produced by splicing of the pre-mRNA at the alternative 5′ splice-site undergoes non-sense mediated RNA decay, wherein the agent modulates processing of the pre-mRNA by modulating splicing at the alternative 5′ splice-site, thereby modulating expression of the target protein; and wherein the target gene is PKD2.

In some embodiments, the agent: (a) binds to a targeted portion of the pre-mRNA; (b) modulates binding of a factor involved in splicing at the alternative 5′ splice-site; or (c) a combination of (a) and (b). In some embodiments, the agent interferes with binding of the factor involved in splicing at the alternative 5′ splice-site to a region of the targeted portion.

In some embodiments, the targeted portion of the pre-mRNA is proximal to the alternative 5′ splice-site. In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of the alternative 5′ splice-site. In some embodiments, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of the alternative 5′ splice-site. In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of the alternative 5′ splice-site. In some embodiments, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of the alternative 5′ splice-site.

In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of genomic site of GRCh38/hg38: chr4 88036480. In some embodiments, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of genomic site of GRCh38/hg38: chr4:88036480. In some embodiments, the targeted portion of the pre-mRNA is at most about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of genomic site of GRCh38/hg38: chr4:88036480. In some embodiments, the targeted portion of the pre-mRNA is at least about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of genomic site of GRCh38/hg38: chr4:88036480.

In some embodiments, the targeted portion of the pre-mRNA is located in a region between the canonical 5′ splice-site and the alternative 5′ splice-site. In some embodiments, the targeted portion of the pre-mRNA is located in an exon region extended by the splicing at the alternative 5′ splice-site. In some embodiments, the targeted portion of the pre-mRNA at least partially overlaps with the alternative 5′ splice-site. In some embodiments, the targeted portion of the pre-mRNA at least partially overlaps with a region upstream or downstream of the alternative 5′ splice-site. In some embodiments, the targeted portion of the pre-mRNA is within an exon region extended by the splicing at the alternative 5′ splice-site. In some embodiments, the targeted portion of the pre-mRNA comprises about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of an exon region extended by the splicing at the alternative 5′ splice-site. In some embodiments, the targeted portion of the pre-mRNA is located in an intronic region between two canonical exons. In some embodiments, the targeted portion of the pre-mRNA is located in one of the two canonical exons. In some embodiments, the targeted portion of the pre-mRNA is located in a region spanning both an intron and a canonical exon.

In some embodiments, the level the target protein in the cell is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell. In some embodiments, modulation of splicing of the pre-mRNA increases production of the processed mRNA encoding the target protein. In some embodiments, the level of processed mRNA encoding the target protein in the cell contacted with the agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell.

In some embodiments, the target protein is the canonical isoform of the protein. In some embodiments, the processed mRNA that is produced by splicing of the pre-mRNA at the alternative 5′ splice-site comprises a premature termination codon (PTC). In some embodiments, the NSASS modulating agent is the composition as described herein.

Described herein, in certain embodiments, is a pharmaceutical composition comprising the composition as described herein; and a pharmaceutically acceptable excipient and/or a delivery vehicle.

Described herein, in certain embodiments, is a method of treating or reducing the likelihood of developing a disease or condition in a subject in need thereof, the method comprising: administering to the subject a pharmaceutical composition to a subject in need thereof, wherein the pharmaceutical composition comprises a composition comprising a non-sense mediated RNA decay alternative splice site (NSASS) modulating agent or a viral vector encoding the agent, wherein the agent modulates expression of a target protein in a cell comprising a pre-mRNA that is transcribed from a target gene and encodes the target protein, wherein the pre-mRNA comprises an alternative 5′ splice-site downstream of a canonical 5′ splice-site, wherein splicing of the pre-mRNA at the alternative 5′ splice-site leads to non-sense mediated RNA decay of the alternatively spliced mRNA, wherein the agent modulates processing of the pre-mRNA by modulating splicing at the alternative 5′ splice-sites; and wherein the target gene is PKD2 and a pharmaceutically acceptable excipient. Described herein, in certain embodiments, is a method of treating or reducing the likelihood of developing a disease or condition in a subject in need thereof, the method comprising: administering to the subject the pharmaceutical composition as described herein.

In some embodiments, the disease is polycystic kidney disease with or without polycystic liver disease, autosomal dominant polycystic kidney disease, or intracranial aneurysm. In some embodiments, the disease or condition is a disease or condition associated with a deficiency in amount or activity of polycystin 2. In some embodiments, the disease or condition is a disease or condition associated with a deficiency in amount or activity of polycystin 1. In some embodiments, the disease or condition is a disease or condition associated with a deficiency in amount or activity of a protein that polycystin 2 functionally augments, compensates for, replaces, or functionally interacts with. In some embodiments, the disease or the condition is caused by a deficient amount or activity of the target protein.

In some embodiments, the agent increases the level of the processed mRNA encoding the target protein in the cell. In some embodiments, the level of processed mRNA encoding the target protein in the cell contacted with the agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell. In some embodiments, the agent increases the expression of the target protein in the cell. In some embodiments, the level the target protein in the cell is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell.

In some embodiments, the method further comprises assessing mRNA levels or expression levels of the target protein. In some embodiments, the method further comprises assessing the subject's genome for at least one genetic mutation associated with the disease. In some embodiments, at least one genetic mutation is within a locus of a gene associated with the disease. In some embodiments, at least one genetic mutation is within a locus associated with expression of a gene associated with the disease. In some embodiments, at least one genetic mutation is within the PKD2 gene locus. In some embodiments, at least one genetic mutation is within the PKD1 gene locus. In some embodiments, at least one genetic mutation is within a locus associated with PKD2 gene expression. In some embodiments, the mutation in PKD1 comprises a mutation in a region of polycystin 1 that interacts with polycystin 2. In some embodiments, the mutation in PKD1 comprises a mutation that interferes the interaction between Polycystin 1 and Polycystin 2. In some embodiments, the mutation in PKD1 comprises a mutation that weakens the interaction between Polycystin 1 and Polycystin 2. In some embodiments, the mutation in PKD1 comprises a mutation that reduces the interaction between Polycystin 1 and Polycystin 2. In some embodiments, the mutation in PKD1 comprises a mutation that blocks the interaction between Polycystin 1 and Polycystin 2. In some embodiments, the mutation in PKD1 is a mutation in a region of polycystin 1 that interacts with polycystin 2. In some embodiments, the mutation in PKD1 is a mutation that interferes the interaction between Polycystin 1 and Polycystin 2. In some embodiments, the mutation in PKD1 is a mutation that weakens the interaction between Polycystin 1 and Polycystin 2. In some embodiments, the mutation in PKD1 is a mutation that reduces the interaction between Polycystin 1 and Polycystin 2. In some embodiments, the mutation in PKD1 is a mutation that blocks the interaction between Polycystin 1 and Polycystin 2.

In some embodiments, the subject is a human. In some embodiments, the subject is a non-human animal. In some embodiments, the subject is a fetus, an embryo, or a child. In some embodiments, the cell or the cells is ex vivo, or in a tissue, or organ ex vivo. In some embodiments, the agent is administered to the subject by intracerebroventricular injection, intraperitoneal injection, intramuscular injection, intrathecal injection, subcutaneous injection, oral administration, synovial injection, intravitreal administration, subretinal injection, topical application, implantation, or intravenous injection. In some embodiments, the method treats the disease or condition.

Described herein, in certain embodiments, is a therapeutic agent for use in the method as described herein. Described herein, in certain embodiments, is a pharmaceutical composition comprising the therapeutic agent as described herein and a pharmaceutically acceptable excipient. Described herein, in certain embodiments, is a method of treating or reducing the likelihood of developing a disease or condition in a subject in need thereof, comprising administering the pharmaceutical composition as described herein by intracerebroventricular injection, intraperitoneal injection, intramuscular injection, intrathecal injection, subcutaneous injection, oral administration, synovial injection, intravitreal administration, subretinal injection, topical application, implantation, or intravenous injection to the subject. In some embodiments, the method treats the subject.

1-77. (canceled)

78. A composition comprising an agent or a vector encoding the agent that (a) binds to a target sequence of a pre-mRNA that is transcribed from a target gene and that comprises a non-sense mediated RNA decay-inducing exon (NMD exon), and (b) modulates splicing of the NMD exon from the pre-mRNA, thereby modulating the level of a processed mRNA that is processed from the pre-mRNA, and modulating expression of a target protein in a cell having the pre-mRNA, wherein the target protein is encoded by a PKD2 gene.

79-184. (canceled)

185. The composition of claim 78, wherein the target sequence of the pre-mRNA comprises a sequence with 8 contiguous nucleic acids of a sequence selected from the group consisting of premrna ENST00000237596.7, premrna ENST00000502363.1, premrna ENST00000506367.1, premrna ENST00000506727.1, premrna ENST00000508588.5, premrna ENST00000511337.5, premrna ENST00000512858.1, transcript ENST00000237596.7, transcript ENST00000502363.1, transcript ENST00000506367.1, transcript ENST00000506727.1, transcript ENST00000508588.5, transcript ENST00000511337.5, transcript ENST00000512858.1, and PKD2 5′ UTR sequence (ENST00000237596.7).

186. The composition of claim 78, wherein the target sequence of the pre-mRNA is within the NMD exon.

187. The composition of claim 78, wherein the target sequence of the pre-mRNA is located in an intronic region between two canonical exonic regions of the pre-mRNA, and wherein the intronic region contains the NMD exon.

188. The composition of claim 187, wherein the NMD exon is defined by genomic sites GRCh38/hg38: chr4:88031085-88031140.

189. The composition of claim 188, wherein the target sequence of the pre-mRNA is closer than 1500 nucleotides, 1000 nucleotides, 800 nucleotides, 700 nucleotides, 600 nucleotides, 500 nucleotides, 400 nucleotides, 300 nucleotides, 200 nucleotides, 100 nucleotides, 80 nucleotides, 70 nucleotides, 60 nucleotides, 50 nucleotides upstream of genomic site of GRCh38/hg38: chr4:88031085.

190. The composition of claim 188, wherein the target sequence of the pre-mRNA is closer than 1500 nucleotides, 1000 nucleotides, 800 nucleotides, 700 nucleotides, 600 nucleotides, 500 nucleotides, 400 nucleotides, 300 nucleotides, 200 nucleotides, 100 nucleotides, 80 nucleotides, 70 nucleotides, 60 nucleotides, 50 nucleotides downstream of genomic site of GRCh38/hg38: chr4:88031085.

191. The composition of claim 188, wherein the target sequence of the pre-mRNA is closer than 1500 nucleotides, 1000 nucleotides, 800 nucleotides, 700 nucleotides, 600 nucleotides, 500 nucleotides, 400 nucleotides, 300 nucleotides, 200 nucleotides, 100 nucleotides, 80 nucleotides, 70 nucleotides, 60 nucleotides, 50 nucleotides upstream of genomic site of GRCh38/hg38: chr4:88031140.

192. The composition of claim 188, wherein the target sequence of the pre-mRNA is closer than 1500 nucleotides, 1000 nucleotides, 800 nucleotides, 700 nucleotides, 600 nucleotides, 500 nucleotides, 400 nucleotides, 300 nucleotides, 200 nucleotides, 100 nucleotides, 80 nucleotides, 70 nucleotides, 60 nucleotides, 50 nucleotides downstream of genomic site of GRCh38/hg38: chr4:88031140.

193. The composition of claim 78, wherein the agent is a non-sense mediated RNA decay alternative splice site (NSASS) modulating agent and the NMD exon is defined by genomic sites GRCh38/hg38: chr4:88036354 88036480.

194. The composition of claim 193, wherein the target sequence of the pre-mRNA is closer than 1500 nucleotides, 1000 nucleotides, 800 nucleotides, 700 nucleotides, 600 nucleotides, 500 nucleotides, 400 nucleotides, 300 nucleotides, 200 nucleotides, 100 nucleotides, 80 nucleotides, 70 nucleotides, 60 nucleotides, 50 nucleotides upstream of genomic site of GRCh38/hg38: chr4 88036480.

195. The composition of claim 193, wherein the target sequence of the pre-mRNA is closer than 1500 nucleotides, 1000 nucleotides, 800 nucleotides, 700 nucleotides, 600 nucleotides, 500 nucleotides, 400 nucleotides, 300 nucleotides, 200 nucleotides, 100 nucleotides, 80 nucleotides, 70 nucleotides, about 60 nucleotides, about 50 nucleotides downstream of genomic site of GRCh38/hg38: chr4:88036480.

196. The composition of claim 78, wherein the composition comprises the agent, and wherein the agent is an antisense oligomer (ASO).

197. The composition of claim 196, wherein the ASO comprises 8, 9, 10, 11, 12, 13, 14, 15, 16, 17 or 18 contiguous nucleic acids of a sequence selected from the group consisting of SEQ ID NOs: 1-226.

198. The composition of claim 196, wherein the ASO comprises a backbone modification comprising a phosphorothioate linkage or a phosphorodiamidate linkage.

199. The composition of claim 196, wherein the ASO comprises a phosphorodiamidate morpholino, a locked nucleic acid, a peptide nucleic acid, a 2′-O-methyl, a 2′-Fluoro, or a 2′-O-methoxyethyl moiety.

200. The composition of claim 78, wherein the composition comprises the vector encoding the agent, and wherein the vector is a viral vector.

201. A pharmaceutical composition comprising the composition of claim 78; and a pharmaceutically acceptable excipient and/or a delivery vehicle.

202. A method of modulating expression of a target protein in a cell having a pre-mRNA that is transcribed from a target gene and that comprises a non-sense mediated RNA decay-inducing exon (NMD exon), the method comprising contacting an agent or a vector encoding the agent to the cell, whereby the agent modulates splicing of the NMD exon from the pre-mRNA, thereby modulating the level of a processed mRNA that is processed from the pre-mRNA, and modulating the expression of the target protein in the cell, wherein the agent binds to a target sequence of the pre-mRNA, and wherein the target protein is encoded by a PKD2 gene.

203. A method of treating or reducing the likelihood of developing a disease or condition in a subject in need thereof by modulating expression of a target protein in a cell of the subject, comprising: contacting an agent or a vector encoding the agent to the cell of the subject, whereby the agent modulates splicing of a non-sense mediated mRNA decay-inducing exon (NMD exon) from a pre-mRNA that is transcribed from a target gene and that comprises the NMD exon, thereby modulating the level of a processed mRNA that is processed from the pre-mRNA, and modulating the expression of the target protein in the cell of the subject, wherein the agent binds to a target sequence of the pre-mRNA, and wherein the target protein encoded by a PKD2 gene.


SEQUENCE LISTING (271 sequences)

SEQ ID NO: 1 - DNA, 18 bp, synthetic construct
  misc_feature 1..18: note = Description of Artificial Sequence: Synthetic oligonucleotide
  source 1..18: mol_type = other DNA
gtactttcaaagttattt

SEQ ID NO: 2 - DNA, 18 bp, synthetic construct
  misc_feature 1..18: note = Description of Artificial Sequence: Synthetic oligonucleotide
  source 1..18: mol_type = other DNA
gagaggtactttcaaagt

SEQ ID NO: 3 - DNA, 18 bp, synthetic construct
  misc_feature 1..18: note = Description of Artificial Sequence: Synthetic oligonucleotide
  source 1..18: mol_type = other DNA
tgatagagaggtactttc

SEQ ID NO: 4 - DNA, 18 bp, synthetic construct
  misc_feature 1..18: note = Description of Artificial Sequence: Synthetic oligonucleotide
  source 1..18: mol_type = other DNA
ttctgtgatagagaggta

SEQ ID NO: 5 - DNA, 18 bp, synthetic construct
  misc_feature 1..18: note = Description of Artificial Sequence: Synthetic oligonucleotide
  source 1..18: mol_type = other DNA
caattttctgtgatagag

SEQ ID NO: 6 - DNA, 18 bp, synthetic construct
  misc_feature 1..18: note = Description of Artificial Sequence: Synthetic oligonucleotide
  source 1..18: mol_type = other DNA
atgaacaattttctgtga

SEQ ID NO: 7 - DNA, 18 bp, synthetic construct
  misc_feature 1..18: note = Description of Artificial Sequence: Synthetic oligonucleotide
  source 1..18: mol_type = other DNA
gccaaatgaacaattttc

SEQ ID NO: 8 - DNA, 18 bp, synthetic construct
  misc_feature 1..18: note = Description of Artificial Sequence: Synthetic oligonucleotide
  source 1..18: mol_type = other DNA
atgaagccaaatgaacaa

SEQ ID NO: 9 - DNA, 18 bp, synthetic construct
  misc_feature 1..18: note = Description of Artificial Sequence: Synthetic oligonucleotide
  source 1..18: mol_type = other DNA
aaatgatgaagccaaatg

SEQ ID NO: 10 - DNA, 18 bp, synthetic construct
  misc_feature 1..18: note = Description of Artificial Sequence: Synthetic oligonucleotide
  source 1..18: mol_type = other DNA
cattgaaatgatgaagcc

SEQ ID NO: 11 - DNA, 18 bp, synthetic construct
  misc_feature 1..18: note = Description of Artificial Sequence: Synthetic oligonucleotide
  source 1..18: mol_type = other DNA
tcatgcattgaaatgatg

SEQ ID NO: 12 - DNA, 18 bp, synthetic construct
  misc_feature 1..18: note = Description of Artificial Sequence: Synthetic oligonucleotide
  source 1..18: mol_type = other DNA
gatactcatgcattgaaa

SEQ ID NO: 13 - DNA, 18 bp, synthetic construct
  misc_feature 1..18: note = Description of Artificial Sequence: Synthetic oligonucleotide
  source 1..18: mol_type = other DNA
ctgtcgatactcatgcat

SEQ ID NO: 14 - DNA, 18 bp, synthetic construct
  misc_feature 1..18: note = Description of Artificial Sequence: Synthetic oligonucleotide
  source 1..18: mol_type = other DNA
aggtcctgtcgatactca

SEQ ID NO: 15 - DNA, 18 bp, synthetic construct
  misc_feature 1..18: note = Description of Artificial Sequence: Synthetic oligonucleotide
  source 1..18: mol_type = other DNA
aaagcaggtcctgtcgat

SEQ ID NO: 16 - DNA, 18 bp, synthetic construct
  misc_feature 1..18: note = Description of Artificial Sequence: Synthetic oligonucleotide
  source 1..18: mol_type = other DNA
aatgcaaagcaggtcctg

SEQ ID NO: 17 - DNA, 18 bp, synthetic construct
  misc_feature 1..18: note = Description of Artificial Sequence: Synthetic oligonucleotide
  source 1..18: mol_type = other DNA
tgttaaatgcaaagcagg

SEQ ID NO: 18 - DNA, 18 bp, synthetic construct
  misc_feature 1..18: note = Description of Artificial Sequence: Synthetic oligonucleotide
  source 1..18: mol_type = other DNA
cacagtgttaaatgcaaa

SEQ ID NO: 19 - DNA, 18 bp, synthetic construct
  misc_feature 1..18: note = Description of Artificial Sequence: Synthetic oligonucleotide
  source 1..18: mol_type = other DNA
tctcacacagtgttaaat

SEQ ID NO: 20 - DNA, 18 bp, synthetic construct
  misc_feature 1..18: note = Description of Artificial Sequence: Synthetic oligonucleotide
  source 1..18: mol_type = other DNA
ttacgtctcacacagtgt

SEQ ID NO: 21 - DNA, 18 bp, synthetic construct
  misc_feature 1..18: note = Description of Artificial Sequence: Synthetic oligonucleotide
  source 1..18: mol_type = other DNA
ataacttacgtctcacac

SEQ ID NO: 22 - DNA, 18 bp, synthetic construct
  misc_feature 1..18: note = Description of Artificial Sequence: Synthetic oligonucleotide
  source 1..18: mol_type = other DNA
tcaccataacttacgtct

SEQ ID NO: 23 - DNA, 18 bp, synthetic construct
  misc_feature 1..18: note = Description of Artificial Sequence: Synthetic oligonucleotide
  source 1..18: mol_type = other DNA
acaactcaccataactta

SEQ ID NO: 24 - DNA, 18 bp, synthetic construct
  misc_feature 1..18: note = Description of Artificial Sequence: Synthetic oligonucleotide
  source 1..18: mol_type = other DNA
ttctaacaactcaccata

SEQ ID NO: 25 - DNA, 18 bp, synthetic construct
  misc_feature 1..18: note = Description of Artificial Sequence: Synthetic oligonucleotide
  source 1..18: mol_type = other DNA
gtaacttctaacaactca

SEQ ID NO: 26 - DNA, 18 bp, synthetic construct
  misc_feature 1..18: note = Description of Artificial Sequence: Synthetic oligonucleotide
  source 1..18: mol_type = other DNA
gaacagtaacttctaaca

SEQ ID NO: 27 - DNA, 18 bp, synthetic construct
  misc_feature 1..18: note = Description of Artificial Sequence: Synthetic oligonucleotide
  source 1..18: mol_type = other DNA
agtaggaacagtaacttc

SEQ ID NO: 28 - DNA, 18 bp, synthetic construct
  misc_feature 1..18: note = Description of Artificial Sequence: Synthetic oligonucleotide
  source 1..18: mol_type = other DNA
ttgagagtaggaacagta

SEQ ID NO: 29 - DNA, 18 bp, synthetic construct
  misc_feature 1..18: note = Description of Artificial Sequence: Synthetic oligonucleotide
  source 1..18: mol_type = other DNA
accccctttgagagtagg

SEQ ID NO: 30 - DNA, 18 bp, synthetic construct
  misc_feature 1..18: note = Description of Artificial Sequence: Synthetic oligonucleotide
  source 1..18: mol_type = other DNA
agtttaccccctttgaga

SEQ ID NO: 31 - DNA, 18 bp, synthetic construct
  misc_feature 1..18: note = Description of Artificial Sequence: Synthetic oligonucleotide
  source 1..18: mol_type = other DNA
atgttagtttaccccctt

SEQ ID NO: 32 - DNA, 18 bp, synthetic construct
  misc_feature 1..18: note = Description of Artificial Sequence: Synthetic oligonucleotide
  source 1..18: mol_type = other DNA
tctcaatgttagtttacc

SEQ ID NO: 33 - DNA, 18 bp, synthetic construct
  misc_feature 1..18: note = Description of Artificial Sequence: Synthetic oligonucleotide
  source 1..18: mol_type = other DNA
aaagttctcaatgttagt

SEQ ID NO: 34 - DNA, 18 bp, synthetic construct
  misc_feature 1..18: note = Description of Artificial Sequence: Synthetic oligonucleotide
  source 1..18: mol_type = other DNA
caggcaaagttctcaatg

SEQ ID NO: 35 - DNA, 18 bp, synthetic construct
  misc_feature 1..18: note = Description of Artificial Sequence: Synthetic oligonucleotide
  source 1..18: mol_type = other DNA
aggcacaggcaaagttct

SEQ ID NO: 36 - DNA, 18 bp, synthetic construct
  misc_feature 1..18: note = Description of Artificial Sequence: Synthetic oligonucleotide
  source 1..18: mol_type = other DNA
gtgcaaggcacaggcaaa

SEQ ID NO: 37 - DNA, 18 bp, synthetic construct
  misc_feature 1..18: note = Description of Artificial Sequence: Synthetic oligonucleotide
  source 1..18: mol_type = other DNA
gcacagtgcaaggcacag

SEQ ID NO: 38 - DNA, 18 bp, synthetic construct
  misc_feature 1..18: note = Description of Artificial Sequence: Synthetic oligonucleotide
  source 1..18: mol_type = other DNA
actcagcacagtgcaagg

SEQ ID NO: 39 - DNA, 18 bp, synthetic construct
  misc_feature 1..18: note = Description of Artificial Sequence: Synthetic oligonucleotide
  source 1..18: mol_type = other DNA
gaaacactcagcacagtg

SEQ ID NO: 40 - DNA, 18 bp, synthetic construct
  misc_feature 1..18: note = Description of Artificial Sequence: Synthetic oligonucleotide
  source 1..18: mol_type = other DNA
gatatgaaacactcagca

SEQ ID NO: 41 - DNA, 18 bp, synthetic construct
  misc_feature 1..18: note = Description of Artificial Sequence: Synthetic oligonucleotide
  source 1..18: mol_type = other DNA
ggtaagatatgaaacact

SEQ ID NO: 42 - DNA, 18 bp, synthetic construct
  misc_feature 1..18: note = Description of Artificial Sequence: Synthetic oligonucleotide
  source 1..18: mol_type = other DNA
aataaggtaagatatgaa

SEQ ID NO: 43 - DNA, 18 bp, synthetic construct
  misc_feature 1..18: note = Description of Artificial Sequence: Synthetic oligonucleotide
  source 1..18: mol_type = other DNA
aattaaataaggtaagat

SEQ ID NO: 44 - DNA, 18 bp, synthetic construct
  misc_feature 1..18: note = Description of Artificial Sequence: Synthetic oligonucleotide
  source 1..18: mol_type = other DNA
atagaaattaaataaggt

SEQ ID NO: 45 - DNA, 18 bp, synthetic construct
  misc_feature 1..18: note = Description of Artificial Sequence: Synthetic oligonucleotide
  source 1..18: mol_type = other DNA
agactatagaaattaaat

SEQ ID NO: 46 - DNA, 18 bp, synthetic construct
  misc_feature 1..18: note = Description of Artificial Sequence: Synthetic oligonucleotide
  source 1..18: mol_type = other DNA
gagttagactatagaaat

SEQ ID NO: 47 - DNA, 18 bp, synthetic construct
  misc_feature 1..18: note = Description of Artificial Sequence: Synthetic oligonucleotide
  source 1..18: mol_type = other DNA
ttatagagttagactata

SEQ ID NO: 48 - DNA, 18 bp, synthetic construct
  misc_feature 1..18: note = Description of Artificial Sequence: Synthetic oligonucleotide
  source 1..18: mol_type = other DNA
ttaccttatagagttaga

SEQ ID NO: 49 - DNA, 18 bp, synthetic construct
  misc_feature 1..18: note = Description of Artificial Sequence: Synthetic oligonucleotide
  source 1..18: mol_type = other DNA
agtacttaccttatagag

SEQ ID NO: 50 - DNA, 18 bp, synthetic construct
  misc_feature 1..18: note = Description of Artificial Sequence: Synthetic oligonucleotide
  source 1..18: mol_type = other DNA
ataaatcgaaccatcgta

SEQ ID NO: 51 - DNA, 18 bp, synthetic construct
  misc_feature 1..18: note = Description of Artificial Sequence: Synthetic oligonucleotide
  source 1..18: mol_type = other DNA
aaatcataaatcgaacca

SEQ ID NO: 52 - DNA, 18 bp, synthetic construct
  misc_feature 1..18: note = Description of Artificial Sequence: Synthetic oligonucleotide
  source 1..18: mol_type = other DNA
ttgaaaaatcataaatcg

SEQ ID NO: 53 - DNA, 18 bp, synthetic construct
  misc_feature 1..18: note = Description of Artificial Sequence: Synthetic oligonucleotide
  source 1..18: mol_type = other DNA
tgaagttgaaaaatcata

SEQ ID NO: 54 - DNA, 18 bp, synthetic construct
  misc_feature 1..18: note = Description of Artificial Sequence: Synthetic oligonucleotide
  source 1..18: mol_type = other DNA
caccatgaagttgaaaaa

SEQ ID NO: 55 - DNA, 18 bp, synthetic construct
  misc_feature 1..18: note = Description of Artificial Sequence: Synthetic oligonucleotide
  source 1..18: mol_type = other DNA
cacatcaccatgaagttg

SEQ ID NO: 56 - DNA, 18 bp, synthetic construct
  misc_feature 1..18: note = Description of Artificial Sequence: Synthetic oligonucleotide
  source 1..18: mol_type = other DNA
actttcacatcaccatga

SEQ ID NO: 57 - DNA, 18 bp, synthetic construct
  misc_feature 1..18: note = Description of Artificial Sequence: Synthetic oligonucleotide
  source 1..18: mol_type = other DNA
gtatcactttcacatcac

SEQ ID NO: 58 - DNA, 18 bp, synthetic construct
  misc_feature 1..18: note = Description of Artificial Sequence: Synthetic oligonucleotide
  source 1..18: mol_type = other DNA
aatgtgtatcactttcac

SEQ ID NO: 59 - DNA, 18 bp, synthetic construct
  misc_feature 1..18: note = Description of Artificial Sequence: Synthetic oligonucleotide
  source 1..18: mol_type = other DNA
tatagaatgtgtatcact

SEQ ID NO: 60 - DNA, 18 bp, synthetic construct
  misc_feature 1..18: note = Description of Artificial Sequence: Synthetic oligonucleotide
  source 1..18: mol_type = other DNA
gtttctatagaatgtgta

SEQ ID NO: 61 - DNA, 18 bp, synthetic construct
  misc_feature 1..18: note = Description of Artificial Sequence: Synthetic oligonucleotide
  source 1..18: mol_type = other DNA
gtgtggtttctatagaat

SEQ ID NO: 62 - DNA, 18 bp, synthetic construct
  misc_feature 1..18: note = Description of Artificial Sequence: Synthetic oligonucleotide
  source 1..18: mol_type = other DNA
tgaaagtgtggtttctat

SEQ ID NO: 63 - DNA, 18 bp, synthetic construct
  misc_feature 1..18: note = Description of Artificial Sequence: Synthetic oligonucleotide
  source 1..18: mol_type = other DNA
aaaattgaaagtgtggtt

SEQ ID NO: 64 - DNA, 18 bp, synthetic construct
  misc_feature 1..18: note = Description of Artificial Sequence: Synthetic oligonucleotide
  source 1..18: mol_type = other DNA
aattcaaaattgaaagtg

SEQ ID NO: 65 - DNA, 18 bp, synthetic construct
  misc_feature 1..18: note = Description of Artificial Sequence: Synthetic oligonucleotide
  source 1..18: mol_type = other DNA
accaaaattcaaaattga

SEQ ID NO: 66 - DNA, 18 bp, synthetic construct
  misc_feature 1..18: note = Description of Artificial Sequence: Synthetic oligonucleotide
  source 1..18: mol_type = other DNA
aaaagaccaaaattcaaa

SEQ ID NO: 67 - DNA, 18 bp, synthetic construct
  misc_feature 1..18: note = Description of Artificial Sequence: Synthetic oligonucleotide
  source 1..18: mol_type = other DNA
ctggaaaaagaccaaaat

SEQ ID NO: 68 - DNA, 18 bp, synthetic construct
  misc_feature 1..18: note = Description of Artificial Sequence: Synthetic oligonucleotide
  source 1..18: mol_type = other DNA
gtagcctggaaaaagacc

SEQ ID NO: 69 - DNA, 18 bp, synthetic construct
  misc_feature 1..18: note = Description of Artificial Sequence: Synthetic oligonucleotide
  source 1..18: mol_type = other DNA
gtatggtagcctggaaaa

SEQ ID NO: 70 - DNA, 18 bp, synthetic construct
  misc_feature 1..18: note = Description of Artificial Sequence: Synthetic oligonucleotide
  source 1..18: mol_type = other DNA
ttagagtatggtagcctg

SEQ ID NO: 71 - DNA, 18 bp, synthetic construct
  misc_feature 1..18: note = Description of Artificial Sequence: Synthetic oligonucleotide
  source 1..18: mol_type = other DNA
tctttagagtatggtagc

SEQ ID NO: 72 - DNA, 18 bp, synthetic construct
  misc_feature 1..18: note = Description of Artificial Sequence: Synthetic oligonucleotide
  source 1..18: mol_type = other DNA
ctctatctttagagtatg

SEQ ID NO: 73 - DNA, 18 bp, synthetic construct
  misc_feature 1..18: note = Description of Artificial Sequence: Synthetic oligonucleotide
  source 1..18: mol_type = other DNA
tgtggctctatctttaga

SEQ ID NO: 74 - DNA, 18 bp, synthetic construct
  misc_feature 1..18: note = Description of Artificial Sequence: Synthetic oligonucleotide
  source 1..18: mol_type = other DNA
ggatctgtggctctatct

SEQ ID NO: 75 - DNA, 18 bp, synthetic construct
  misc_feature 1..18: note = Description of Artificial Sequence: Synthetic oligonucleotide
  source 1..18: mol_type = other DNA
gactgggatctgtggctc

SEQ ID NO: 76 - DNA, 18 bp, synthetic construct
  misc_feature 1..18: note = Description of Artificial Sequence: Synthetic oligonucleotide
  source 1..18: mol_type = other DNA
acatggctgactgggatc

SEQ ID NO: 77 - DNA, 18 bp, synthetic construct
  misc_feature 1..18: note = Description of Artificial Sequence: Synthetic oligonucleotide
  source 1..18: mol_type = other DNA
taatcacatggctgactg

SEQ ID NO: 78 - DNA, 18 bp, synthetic construct
  misc_feature 1..18: note = Description of Artificial Sequence: Synthetic oligonucleotide
  source 1..18: mol_type = other DNA
cctcataatcacatggct

SEQ ID NO: 79 - DNA, 18 bp, synthetic construct
  misc_feature 1..18: note = Description of Artificial Sequence: Synthetic oligonucleotide
  source 1..18: mol_type = other DNA
taccctcataatcacatg

SEQ ID NO: 80 - DNA, 18 bp, synthetic construct
  misc_feature 1..18: note = Description of Artificial Sequence: Synthetic oligonucleotide
  source 1..18: mol_type = other DNA
tcgcttaccctcataatc

SEQ ID NO: 81 - DNA, 18 bp, synthetic construct
  misc_feature 1..18: note = Description of Artificial Sequence: Synthetic oligonucleotide
  source 1..18: mol_type = other DNA
attggtcgcttaccctca

SEQ ID NO: 82 - DNA, 18 bp, synthetic construct
  misc_feature 1..18: note = Description of Artificial Sequence: Synthetic oligonucleotide
  source 1..18: mol_type = other DNA
agagtattggtcgcttac

SEQ ID NO: 83 - DNA, 18 bp, synthetic construct
  misc_feature 1..18: note = Description of Artificial Sequence: Synthetic oligonucleotide
  source 1..18: mol_type = other DNA
actgtagagtattggtcg

SEQ ID NO: 84 - DNA, 18 bp, synthetic construct
  misc_feature 1..18: note = Description of Artificial Sequence: Synthetic oligonucleotide
  source 1..18: mol_type = other DNA
aatacactgtagagtatt

SEQ ID NO: 85 - DNA, 18 bp, synthetic construct
  misc_feature 1..18: note = Description of Artificial Sequence: Synthetic oligonucleotide
  source 1..18: mol_type = other DNA
aatacaatacactgtaga

SEQ ID NO: 86 - DNA, 18 bp, synthetic construct
  misc_feature 1..18: note = Description of Artificial Sequence: Synthetic oligonucleotide
  source 1..18: mol_type = other DNA
ctggcaatacaatacact

SEQ ID NO: 87 - DNA, 18 bp, synthetic construct
  misc_feature 1..18: note = Description of Artificial Sequence: Synthetic oligonucleotide
  source 1..18: mol_type = other DNA
accatctggcaatacaat

SEQ ID NO: 88 - DNA, 18 bp, synthetic construct
  misc_feature 1..18: note = Description of Artificial Sequence: Synthetic oligonucleotide
  source 1..18: mol_type = other DNA
gcaaaaccatctggcaat

SEQ ID NO: 89 - DNA, 18 bp, synthetic construct
  misc_feature 1..18: note = Description of Artificial Sequence: Synthetic oligonucleotide
  source 1..18: mol_type = other DNA
gttgggcaaaaccatctg

SEQ ID NO: 90 - DNA, 18 bp, synthetic construct
  misc_feature 1..18: note = Description of Artificial Sequence: Synthetic oligonucleotide
  source 1..18: mol_type = other DNA
ggctagttgggcaaaacc

SEQ ID NO: 91 - DNA, 18 bp, synthetic construct
  misc_feature 1..18: note = Description of Artificial Sequence: Synthetic oligonucleotide
  source 1..18: mol_type = other DNA
cattaggctagttgggca

SEQ ID NO: 92 - DNA, 18 bp, synthetic construct
  misc_feature 1..18: note = Description of Artificial Sequence: Synthetic oligonucleotide
  source 1..18: mol_type = other DNA
acttacattaggctagtt

SEQ ID NO: 93 - DNA, 18 bp, synthetic construct
  misc_feature 1..18: note = Description of Artificial Sequence: Synthetic oligonucleotide
  source 1..18: mol_type = other DNA
agaatacttacattaggc

SEQ ID NO: 94 - DNA, 18 bp, synthetic construct
  misc_feature 1..18: note = Description of Artificial Sequence: Synthetic oligonucleotide
  source 1..18: mol_type = other DNA
tgtttagaatacttacat

SEQ ID NO: 95 - DNA, 18 bp, synthetic construct
  misc_feature 1..18: note = Description of Artificial Sequence: Synthetic oligonucleotide
  source 1..18: mol_type = other DNA
aaacatgtttagaatact

SEQ ID NO: 96 - DNA, 18 bp, synthetic construct
  misc_feature 1..18: note = Description of Artificial Sequence: Synthetic oligonucleotide
  source 1..18: mol_type = other DNA
accttaaacatgtttaga

SEQ ID NO: 97 - DNA, 18 bp, synthetic construct
  misc_feature 1..18: note = Description of Artificial Sequence: Synthetic oligonucleotide
  source 1..18: mol_type = other DNA
ggcctaccttaaacatgt

SEQ ID NO: 98 - DNA, 18 bp, synthetic construct
  misc_feature 1..18: note = Description of Artificial Sequence: Synthetic oligonucleotide
  source 1..18: mol_type = other DNA
agcctggcctaccttaaa

SEQ ID NO: 99 - DNA, 18 bp, synthetic construct
  misc_feature 1..18: note = Description of Artificial Sequence: Synthetic oligonucleotide
  source 1..18: mol_type = other DNA
agcttagcctggcctacc

SEQ ID NO: 100 - DNA, 18 bp, synthetic construct
  misc_feature 1..18: note = Description of Artificial Sequence: Synthetic oligonucleotide
  source 1..18: mol_type = other DNA
tttcttcccggcgcccgc

SEQ ID NO: 101 - DNA, 18 bp, synthetic construct
  misc_feature 1..18: note = Description of Artificial Sequence: Synthetic oligonucleotide
  source 1..18: mol_type = other DNA
cctttcttcccggcgccc

SEQ ID NO: 102 - DNA, 18 bp, synthetic construct
  misc_feature 1..18: note = Description of Artificial Sequence: Synthetic oligonucleotide
  source 1..18: mol_type = other DNA
ttcctttcttcccggcgc

SEQ ID NO: 103 - DNA, 18 bp, synthetic construct
  misc_feature 1..18: note = Description of Artificial Sequence: Synthetic oligonucleotide
  source 1..18: mol_type = other DNA
tgttcctttcttcccggc

SEQ ID NO: 104 - DNA, 18 bp, synthetic construct
  misc_feature 1..18: note = Description of Artificial Sequence: Synthetic oligonucleotide
  source 1..18: mol_type = other DNA
catgttcctttcttcccg

SEQ ID NO: 105 - DNA, 18 bp, synthetic construct
  misc_feature 1..18: note = Description of Artificial Sequence: Synthetic oligonucleotide
  source 1..18: mol_type = other DNA
gccatgttcctttcttcc

SEQ ID NO: 106 - DNA, 18 bp, synthetic construct
  misc_feature 1..18: note = Description of Artificial Sequence: Synthetic oligonucleotide
  source 1..18: mol_type = other DNA
gagccatgttcctttctt

SEQ ID NO: 107 - DNA, 18 bp, synthetic construct
  misc_feature 1..18: note = Description of Artificial Sequence: Synthetic oligonucleotide
  source 1..18: mol_type = other DNA
aggagccatgttcctttc

SEQ ID NO: 108 - DNA, 18 bp, synthetic construct
  misc_feature 1..18: note = Description of Artificial Sequence: Synthetic oligonucleotide
  source 1..18: mol_type = other DNA
tcaggagccatgttcctt

SEQ ID NO: 109 - DNA, 18 bp, synthetic construct
  misc_feature 1..18: note = Description of Artificial Sequence: Synthetic oligonucleotide
  source 1..18: mol_type = other DNA
cctcaggagccatgttcc

SEQ ID NO: 110 - DNA, 18 bp, synthetic construct
  misc_feature 1..18: note = Description of Artificial Sequence: Synthetic oligonucleotide
  source 1..18: mol_type = other DNA
cgcctcaggagccatgtt

SEQ ID NO: 111 - DNA, 18 bp, synthetic construct
  misc_feature 1..18: note = Description of Artificial Sequence: Synthetic oligonucleotide
  source 1..18: mol_type = other DNA
tgcgcctcaggagccatg

SEQ ID NO: 112 - DNA, 18 bp, synthetic construct
  misc_feature 1..18: note = Description of Artificial Sequence: Synthetic oligonucleotide
  source 1..18: mol_type = other DNA
tgtgcgcctcaggagcca

SEQ ID NO: 113 - DNA, 18 bp, synthetic construct
  misc_feature 1..18: note = Description of Artificial Sequence: Synthetic oligonucleotide
  source 1..18: mol_type = other DNA
ctgtgcgcctcaggagcc

SEQ ID NO: 114 - RNA, 18 nt, synthetic construct
  misc_feature 1..18: note = Description of Artificial Sequence: Synthetic oligonucleotide
  source 1..18: mol_type = other RNA
gtactttcaaagttattt

SEQ ID NO: 115 - RNA, 18 nt, synthetic construct
  misc_feature 1..18: note = Description of Artificial Sequence: Synthetic oligonucleotide
  source 1..18: mol_type = other RNA
gagaggtactttcaaagt

SEQ ID NO: 116 - RNA, 18 nt, synthetic construct
  misc_feature 1..18: note = Description of Artificial Sequence: Synthetic oligonucleotide
  source 1..18: mol_type = other RNA
tgatagagaggtactttc

SEQ ID NO: 117 - RNA, 18 nt, synthetic construct
  misc_feature 1..18: note = Description of Artificial Sequence: Synthetic oligonucleotide
  source 1..18: mol_type = other RNA
ttctgtgatagagaggta

SEQ ID NO: 118 - RNA, 18 nt, synthetic construct
  misc_feature 1..18: note = Description of Artificial Sequence: Synthetic oligonucleotide
  source 1..18: mol_type = other RNA
caattttctgtgatagag

SEQ ID NO: 119 - RNA, 18 nt, synthetic construct
  misc_feature 1..18: note = Description of Artificial Sequence: Synthetic oligonucleotide
  source 1..18: mol_type = other RNA
atgaacaattttctgtga

SEQ ID NO: 120 - RNA, 18 nt, synthetic construct
  misc_feature 1..18: note = Description of Artificial Sequence: Synthetic oligonucleotide
  source 1..18: mol_type = other RNA
gccaaatgaacaattttc

SEQ ID NO: 121 - RNA, 18 nt, synthetic construct
  misc_feature 1..18: note = Description of Artificial Sequence: Synthetic oligonucleotide
  source 1..18: mol_type = other RNA
atgaagccaaatgaacaa

SEQ ID NO: 122 - RNA, 18 nt, synthetic construct
  misc_feature 1..18: note = Description of Artificial Sequence: Synthetic oligonucleotide
  source 1..18: mol_type = other RNA
aaatgatgaagccaaatg

SEQ ID NO: 123 - RNA, 18 nt, synthetic construct
  misc_feature 1..18: note = Description of Artificial Sequence: Synthetic oligonucleotide
  source 1..18: mol_type = other RNA
cattgaaatgatgaagcc

SEQ ID NO: 124 - RNA, 18 nt, synthetic construct
  misc_feature 1..18: note = Description of Artificial Sequence: Synthetic oligonucleotide
  source 1..18: mol_type = other RNA
tcatgcattgaaatgatg

SEQ ID NO: 125 - RNA, 18 nt, synthetic construct
  misc_feature 1..18: note = Description of Artificial Sequence: Synthetic oligonucleotide
  source 1..18: mol_type = other RNA
gatactcatgcattgaaa

SEQ ID NO: 126 - RNA, 18 nt, synthetic construct
  misc_feature 1..18: note = Description of Artificial Sequence: Synthetic oligonucleotide
  source 1..18: mol_type = other RNA
ctgtcgatactcatgcat

SEQ ID NO: 127 - RNA, 18 nt, synthetic construct
  misc_feature 1..18: note = Description of Artificial Sequence: Synthetic oligonucleotide
  source 1..18: mol_type = other RNA
aggtcctgtcgatactca

SEQ ID NO: 128 - RNA, 18 nt, synthetic construct
  misc_feature 1..18: note = Description of Artificial Sequence: Synthetic oligonucleotide
  source 1..18: mol_type = other RNA
aaagcaggtcctgtcgat

SEQ ID NO: 129 - RNA, 18 nt, synthetic construct
  misc_feature 1..18: note = Description of Artificial Sequence: Synthetic oligonucleotide
  source 1..18: mol_type = other RNA
aatgcaaagcaggtcctg

SEQ ID NO: 130 - RNA, 18 nt, synthetic construct
  misc_feature 1..18: note = Description of Artificial Sequence: Synthetic oligonucleotide
  source 1..18: mol_type = other RNA
tgttaaatgcaaagcagg

SEQ ID NO: 131 - RNA, 18 nt, synthetic construct
  misc_feature 1..18: note = Description of Artificial Sequence: Synthetic oligonucleotide
  source 1..18: mol_type = other RNA
cacagtgttaaatgcaaa

SEQ ID NO: 132 - RNA, 18 nt, synthetic construct
  misc_feature 1..18: note = Description of Artificial Sequence: Synthetic oligonucleotide
  source 1..18: mol_type = other RNA
tctcacacagtgttaaat

SEQ ID NO: 133 - RNA, 18 nt, synthetic construct
  misc_feature 1..18: note = Description of Artificial Sequence: Synthetic oligonucleotide
  source 1..18: mol_type = other RNA
ttacgtctcacacagtgt

SEQ ID NO: 134 - RNA, 18 nt, synthetic construct
  misc_feature 1..18: note = Description of Artificial Sequence: Synthetic oligonucleotide
  source 1..18: mol_type = other RNA
ataacttacgtctcacac

SEQ ID NO: 135 - RNA, 18 nt, synthetic construct
  misc_feature 1..18: note = Description of Artificial Sequence: Synthetic oligonucleotide
  source 1..18: mol_type = other RNA
tcaccataacttacgtct

SEQ ID NO: 136 - RNA, 18 nt, synthetic construct
  misc_feature 1..18: note = Description of Artificial Sequence: Synthetic oligonucleotide
  source 1..18: mol_type = other RNA
acaactcaccataactta

SEQ ID NO: 137 - RNA, 18 nt, synthetic construct
  misc_feature 1..18: note = Description of Artificial Sequence: Synthetic oligonucleotide
  source 1..18: mol_type = other RNA
ttctaacaactcaccata

SEQ ID NO: 138 - RNA, 18 nt, synthetic construct
  misc_feature 1..18: note = Description of Artificial Sequence: Synthetic oligonucleotide
  source 1..18: mol_type = other RNA
gtaacttctaacaactca

SEQ ID NO: 139 - RNA, 18 nt, synthetic construct
  misc_feature 1..18: note = Description of Artificial Sequence: Synthetic oligonucleotide
  source 1..18: mol_type = other RNA
gaacagtaacttctaaca

SEQ ID NO: 140 - RNA, 18 nt, synthetic construct
  misc_feature 1..18: note = Description of Artificial Sequence: Synthetic oligonucleotide
  source 1..18: mol_type = other RNA
agtaggaacagtaacttc

SEQ ID NO: 141 - RNA, 18 nt, synthetic construct
  misc_feature 1..18: note = Description of Artificial Sequence: Synthetic oligonucleotide
  source 1..18: mol_type = other RNA
ttgagagtaggaacagta

SEQ ID NO: 142 - RNA, 18 nt, synthetic construct
  misc_feature 1..18: note = Description of Artificial Sequence: Synthetic oligonucleotide
  source 1..18: mol_type = other RNA
accccctttgagagtagg

SEQ ID NO: 143 - RNA, 18 nt, synthetic construct
  misc_feature 1..18: note = Description of Artificial Sequence: Synthetic oligonucleotide
  source 1..18: mol_type = other RNA
agtttaccccctttgaga

SEQ ID NO: 144 - RNA, 18 nt, synthetic construct
  misc_feature 1..18: note = Description of Artificial Sequence: Synthetic oligonucleotide
  source 1..18: mol_type = other RNA
atgttagtttaccccctt

SEQ ID NO: 145 - RNA, 18 nt, synthetic construct
  misc_feature 1..18: note = Description of Artificial Sequence: Synthetic oligonucleotide
  source 1..18: mol_type = other RNA
tctcaatgttagtttacc

SEQ ID NO: 146 - RNA, 18 nt, synthetic construct
  misc_feature 1..18: note = Description of Artificial Sequence: Synthetic oligonucleotide
  source 1..18: mol_type = other RNA
aaagttctcaatgttagt

SEQ ID NO: 147 - RNA, 18 nt, synthetic construct
  misc_feature 1..18: note = Description of Artificial Sequence: Synthetic oligonucleotide
  source 1..18: mol_type = other RNA
caggcaaagttctcaatg

SEQ ID NO: 148 - RNA, 18 nt, synthetic construct
  misc_feature 1..18: note = Description of Artificial Sequence: Synthetic oligonucleotide
  source 1..18: mol_type = other RNA
aggcacaggcaaagttct

SEQ ID NO: 149 - RNA, 18 nt, synthetic construct
  misc_feature 1..18: note = Description of Artificial Sequence: Synthetic oligonucleotide
  source 1..18: mol_type = other RNA
gtgcaaggcacaggcaaa

SEQ ID NO: 150 - RNA, 18 nt, synthetic construct
  misc_feature 1..18: note = Description of Artificial Sequence: Synthetic oligonucleotide
  source 1..18: mol_type = other RNA
gcacagtgcaaggcacag

SEQ ID NO: 151 - RNA, 18 nt, synthetic construct
  misc_feature 1..18: note = Description of Artificial Sequence: Synthetic oligonucleotide
  source 1..18: mol_type = other RNA
actcagcacagtgcaagg

SEQ ID NO: 152 - RNA, 18 nt, synthetic construct
  misc_feature 1..18: note = Description of Artificial Sequence: Synthetic oligonucleotide
  source 1..18: mol_type = other RNA
gaaacactcagcacagtg

SEQ ID NO: 153 - RNA, 18 nt, synthetic construct
  misc_feature 1..18: note = Description of Artificial Sequence: Synthetic oligonucleotide
  source 1..18: mol_type = other RNA
gatatgaaacactcagca

SEQ ID NO: 154 - RNA, 18 nt, synthetic construct
  misc_feature 1..18: note = Description of Artificial Sequence: Synthetic oligonucleotide
  source 1..18: mol_type = other RNA
ggtaagatatgaaacact

SEQ ID NO: 155 - RNA, 18 nt, synthetic construct
  misc_feature 1..18: note = Description of Artificial Sequence: Synthetic oligonucleotide
  source 1..18: mol_type = other RNA
aataaggtaagatatgaa

SEQ ID NO: 156 - RNA, 18 nt, synthetic construct
  misc_feature 1..18: note = Description of Artificial Sequence: Synthetic oligonucleotide
  source 1..18: mol_type = other RNA
aattaaataaggtaagat

SEQ ID NO: 157 - RNA, 18 nt, synthetic construct
  misc_feature 1..18: note = Description of Artificial Sequence: Synthetic oligonucleotide
  source 1..18: mol_type = other RNA
atagaaattaaataaggt

SEQ ID NO: 158 - RNA, 18 nt, synthetic construct
  misc_feature 1..18: note = Description of Artificial Sequence: Synthetic oligonucleotide
  source 1..18: mol_type = other RNA
agactatagaaattaaat

SEQ ID NO: 159 - RNA, 18 nt, synthetic construct
  misc_feature 1..18: note = Description of Artificial Sequence: Synthetic oligonucleotide
  source 1..18: mol_type = other RNA
gagttagactatagaaat

SEQ ID NO: 160 - RNA, 18 nt, synthetic construct
  misc_feature 1..18: note = Description of Artificial Sequence: Synthetic oligonucleotide
  source 1..18: mol_type = other RNA
ttatagagttagactata

SEQ ID NO: 161 - RNA, 18 nt, synthetic construct
  misc_feature 1..18: note = Description of Artificial Sequence: Synthetic oligonucleotide
  source 1..18: mol_type = other RNA
ttaccttatagagttaga

SEQ ID NO: 162 - RNA, 18 nt, synthetic construct
  misc_feature 1..18: note = Description of Artificial Sequence: Synthetic oligonucleotide
  source 1..18: mol_type = other RNA
agtacttaccttatagag

SEQ ID NO: 163 - RNA, 18 nt, synthetic construct
  misc_feature 1..18: note = Description of Artificial Sequence: Synthetic oligonucleotide
  source 1..18: mol_type = other RNA
ataaatcgaaccatcgta

SEQ ID NO: 164 - RNA, 18 nt, synthetic construct
  misc_feature 1..18: note = Description of Artificial Sequence: Synthetic oligonucleotide
  source 1..18: mol_type = other RNA
aaatcataaatcgaacca

SEQ ID NO: 165 - RNA, 18 nt, synthetic construct
  misc_feature 1..18: note = Description of Artificial Sequence: Synthetic oligonucleotide
  source 1..18: mol_type = other RNA
ttgaaaaatcataaatcg

SEQ ID NO: 166 - RNA, 18 nt, synthetic construct
  misc_feature 1..18: note = Description of Artificial Sequence: Synthetic oligonucleotide
  source 1..18: mol_type = other RNA
tgaagttgaaaaatcata

SEQ ID NO: 167 - RNA, 18 nt, synthetic construct
  misc_feature 1..18: note = Description of Artificial Sequence: Synthetic oligonucleotide
  source 1..18: mol_type = other RNA
caccatgaagttgaaaaa

SEQ ID NO: 168 - RNA, 18 nt, synthetic construct
  misc_feature 1..18: note = Description of Artificial Sequence: Synthetic oligonucleotide
  source 1..18: mol_type = other RNA
cacatcaccatgaagttg

SEQ ID NO: 169 - RNA, 18 nt, synthetic construct
  misc_feature 1..18: note = Description of Artificial Sequence: Synthetic oligonucleotide
  source 1..18: mol_type = other RNA
actttcacatcaccatga

SEQ ID NO: 170 - RNA, 18 nt, synthetic construct
  misc_feature 1..18: note = Description of Artificial Sequence: Synthetic oligonucleotide
  source 1..18: mol_type = other RNA
gtatcactttcacatcac

SEQ ID NO: 171 - RNA, 18 nt, synthetic construct
  misc_feature 1..18: note = Description of Artificial Sequence: Synthetic oligonucleotide
  source 1..18: mol_type = other RNA
aatgtgtatcactttcac

SEQ ID NO: 172 - RNA, 18 nt, synthetic construct
  misc_feature 1..18: note = Description of Artificial Sequence: Synthetic oligonucleotide
  source 1..18: mol_type = other RNA
tatagaatgtgtatcact

SEQ ID NO: 173 - RNA, 18 nt, synthetic construct
  misc_feature 1..18: note = Description of Artificial Sequence: Synthetic oligonucleotide
  source 1..18: mol_type = other RNA
gtttctatagaatgtgta

SEQ ID NO: 174 - RNA, 18 nt, synthetic construct
  misc_feature 1..18: note = Description of Artificial Sequence: Synthetic oligonucleotide
  source 1..18: mol_type = other RNA
gtgtggtttctatagaat

SEQ ID NO: 175 - RNA, 18 nt, synthetic construct
  misc_feature 1..18: note = Description of Artificial Sequence: Synthetic oligonucleotide
  source 1..18: mol_type = other RNA
tgaaagtgtggtttctat

SEQ ID NO: 176 - RNA, 18 nt, synthetic construct
  misc_feature 1..18: note = Description of Artificial Sequence: Synthetic oligonucleotide
  source 1..18: mol_type = other RNA
aaaattgaaagtgtggtt

SEQ ID NO: 177 - RNA, 18 nt, synthetic construct
  misc_feature 1..18: note = Description of Artificial Sequence: Synthetic oligonucleotide
  source 1..18: mol_type = other RNA
aattcaaaattgaaagtg

SEQ ID NO: 178 - RNA, 18 nt, synthetic construct
  misc_feature 1..18: note = Description of Artificial Sequence: Synthetic oligonucleotide
  source 1..18: mol_type = other RNA
accaaaattcaaaattga

SEQ ID NO: 179 - RNA, 18 nt, synthetic construct
  misc_feature 1..18: note = Description of Artificial Sequence: Synthetic oligonucleotide
  source 1..18: mol_type = other RNA
aaaagaccaaaattcaaa

SEQ ID NO: 180 - RNA, 18 nt, synthetic construct
  misc_feature 1..18: note = Description of Artificial Sequence: Synthetic oligonucleotide
  source 1..18: mol_type = other RNA
ctggaaaaagaccaaaat

SEQ ID NO: 181 - RNA, 18 nt, synthetic construct
  misc_feature 1..18: note = Description of Artificial Sequence: Synthetic oligonucleotide
  source 1..18: mol_type = other RNA
gtagcctggaaaaagacc

SEQ ID NO: 182 - RNA, 18 nt, synthetic construct
  misc_feature 1..18: note = Description of Artificial Sequence: Synthetic oligonucleotide
  source 1..18: mol_type = other RNA
gtatggtagcctggaaaa

SEQ ID NO: 183 - RNA, 18 nt, synthetic construct
  misc_feature 1..18: note = Description of Artificial Sequence: Synthetic oligonucleotide
  source 1..18: mol_type = other RNA
ttagagtatggtagcctg

SEQ ID NO: 184 - RNA, 18 nt, synthetic construct
  misc_feature 1..18: note = Description of Artificial Sequence: Synthetic oligonucleotide
  source 1..18: mol_type = other RNA
tctttagagtatggtagc

SEQ ID NO: 185 - RNA, 18 nt, synthetic construct
  misc_feature 1..18: note = Description of Artificial Sequence: Synthetic oligonucleotide
  source 1..18: mol_type = other RNA
ctctatctttagagtatg

SEQ ID NO: 186 - RNA, 18 nt, synthetic construct
  misc_feature 1..18: note = Description of Artificial Sequence: Synthetic oligonucleotide
  source 1..18: mol_type = other RNA
tgtggctctatctttaga

SEQ ID NO: 187 - RNA, 18 nt, synthetic construct
  misc_feature 1..18: note = Description of Artificial Sequence: Synthetic oligonucleotide
  source 1..18: mol_type = other RNA
ggatctgtggctctatct

SEQ ID NO: 188 - RNA, 18 nt, synthetic construct
  misc_feature 1..18: note = Description of Artificial Sequence: Synthetic oligonucleotide
  source 1..18: mol_type = other RNA
gactgggatctgtggctc

SEQ ID NO: 189 - RNA, 18 nt, synthetic construct
  misc_feature 1..18: note = Description of Artificial Sequence: Synthetic oligonucleotide
  source 1..18: mol_type = other RNA
acatggctgactgggatc

SEQ ID NO: 190 - RNA, 18 nt, synthetic construct
  misc_feature 1..18: note = Description of Artificial Sequence: Synthetic oligonucleotide
  source 1..18: mol_type = other RNA
taatcacatggctgactg

SEQ ID NO: 191 - RNA, 18 nt, synthetic construct
  misc_feature 1..18: note = Description of Artificial Sequence: Synthetic oligonucleotide
  source 1..18: mol_type = other RNA
cctcataatcacatggct

SEQ ID NO: 192 - RNA, 18 nt, synthetic construct
  misc_feature 1..18: note = Description of Artificial Sequence: Synthetic oligonucleotide
  source 1..18: mol_type = other RNA
taccctcataatcacatg

SEQ ID NO: 193 - RNA, 18 nt, synthetic construct
  misc_feature 1..18: note = Description of Artificial Sequence: Synthetic oligonucleotide
  source 1..18: mol_type = other RNA
tcgcttaccctcataatc

SEQ ID NO: 194 - RNA, 18 nt, synthetic construct
  misc_feature 1..18: note = Description of Artificial Sequence: Synthetic oligonucleotide
  source 1..18: mol_type = other RNA
attggtcgcttaccctca

SEQ ID NO: 195 - RNA, 18 nt, synthetic construct
  misc_feature 1..18: note = Description of Artificial Sequence: Synthetic oligonucleotide
  source 1..18: mol_type = other RNA
agagtattggtcgcttac

SEQ ID NO: 196 - RNA, 18 nt, synthetic construct
  misc_feature 1..18: note = Description of Artificial Sequence: Synthetic oligonucleotide
  source 1..18: mol_type = other RNA
actgtagagtattggtcg

SEQ ID NO: 197 - RNA, 18 nt, synthetic construct
  misc_feature 1..18: note = Description of Artificial Sequence: Synthetic oligonucleotide
  source 1..18: mol_type = other RNA
aatacactgtagagtatt

SEQ ID NO: 198 - RNA, 18 nt, synthetic construct
  misc_feature 1..18: note = Description of Artificial Sequence: Synthetic oligonucleotide
  source 1..18: mol_type = other RNA
aatacaatacactgtaga

SEQ ID NO: 199 - RNA, 18 nt, synthetic construct
  misc_feature 1..18: note = Description of Artificial Sequence: Synthetic oligonucleotide
  source 1..18: mol_type = other RNA
ctggcaatacaatacact

SEQ ID NO: 200 - RNA, 18 nt, synthetic construct
  misc_feature 1..18: note = Description of Artificial Sequence: Synthetic oligonucleotide
  source 1..18: mol_type = other RNA
accatctggcaatacaat

SEQ ID NO: 201 - RNA, 18 nt, synthetic construct
  misc_feature 1..18: note = Description of Artificial Sequence: Synthetic oligonucleotide
  source 1..18: mol_type = other RNA
gcaaaaccatctggcaat

SEQ ID NO: 202 - RNA, 18 nt, synthetic construct
  misc_feature 1..18: note = Description of Artificial Sequence: Synthetic oligonucleotide
  source 1..18: mol_type = other RNA
gttgggcaaaaccatctg

SEQ ID NO: 203 - RNA, 18 nt, synthetic construct
  misc_feature 1..18: note = Description of Artificial Sequence: Synthetic oligonucleotide
  source 1..18: mol_type = other RNA
ggctagttgggcaaaacc

SEQ ID NO: 204 - RNA, 18 nt, synthetic construct
  misc_feature 1..18: note = Description of Artificial Sequence: Synthetic oligonucleotide
  source 1..18: mol_type = other RNA
cattaggctagttgggca

SEQ ID NO: 205 - RNA, 18 nt, synthetic construct
  misc_feature 1..18: note = Description of Artificial Sequence: Synthetic oligonucleotide
  source 1..18: mol_type = other RNA
acttacattaggctagtt

SEQ ID NO: 206 - RNA, 18 nt, synthetic construct
  misc_feature 1..18: note = Description of Artificial Sequence: Synthetic oligonucleotide
  source 1..18: mol_type = other RNA
agaatacttacattaggc

SEQ ID NO: 207 - RNA, 18 nt, synthetic construct
  misc_feature 1..18: note = Description of Artificial Sequence: Synthetic oligonucleotide
  source 1..18: mol_type = other RNA
tgtttagaatacttacat

SEQ ID NO: 208 - RNA, 18 nt, synthetic construct
  misc_feature 1..18: note = Description of Artificial Sequence: Synthetic oligonucleotide
  source 1..18: mol_type = other RNA
aaacatgtttagaatact

SEQ ID NO: 209 - RNA, 18 nt, synthetic construct
  misc_feature 1..18: note = Description of Artificial Sequence: Synthetic oligonucleotide
  source 1..18: mol_type = other RNA
accttaaacatgtttaga

SEQ ID NO: 210 - RNA, 18 nt, synthetic construct
  misc_feature 1..18: note = Description of Artificial Sequence: Synthetic oligonucleotide
  source 1..18: mol_type = other RNA
ggcctaccttaaacatgt

SEQ ID NO: 211 - RNA, 18 nt, synthetic construct
  misc_feature 1..18: note = Description of Artificial Sequence: Synthetic oligonucleotide
  source 1..18: mol_type = other RNA
agcctggcctaccttaaa

SEQ ID NO: 212 - RNA, 18 nt, synthetic construct
  misc_feature 1..18: note = Description of Artificial Sequence: Synthetic oligonucleotide
  source 1..18: mol_type = other RNA
agcttagcctggcctacc

SEQ ID NO: 213 - RNA, 18 nt, synthetic construct
  misc_feature 1..18: note = Description of Artificial Sequence: Synthetic oligonucleotide
  source 1..18: mol_type = other RNA
tttcttcccggcgcccgc

SEQ ID NO: 214 - RNA, 18 nt, synthetic construct
  misc_feature 1..18: note = Description of Artificial Sequence: Synthetic oligonucleotide
  source 1..18: mol_type = other RNA
cctttcttcccggcgccc

SEQ ID NO: 215 - RNA, 18 nt, synthetic construct
  misc_feature 1..18: note = Description of Artificial Sequence: Synthetic oligonucleotide
  source 1..18: mol_type = other RNA
ttcctttcttcccggcgc

SEQ ID NO: 216 - RNA, 18 nt, synthetic construct
  misc_feature 1..18: note = Description of Artificial Sequence: Synthetic oligonucleotide
  source 1..18: mol_type = other RNA
tgttcctttcttcccggc

SEQ ID NO: 217 - RNA, 18 nt, synthetic construct
  misc_feature 1..18: note = Description of Artificial Sequence: Synthetic oligonucleotide
  source 1..18: mol_type = other RNA
catgttcctttcttcccg

SEQ ID NO: 218 - RNA, 18 nt, synthetic construct
  misc_feature 1..18: note = Description of Artificial Sequence: Synthetic oligonucleotide
  source 1..18: mol_type = other RNA
gccatgttcctttcttcc

SEQ ID NO: 219 - RNA, 18 nt, synthetic construct
  misc_feature 1..18: note = Description of Artificial Sequence: Synthetic oligonucleotide
  source 1..18: mol_type = other RNA
gagccatgttcctttctt

SEQ ID NO: 220 - RNA, 18 nt, synthetic construct
  misc_feature 1..18: note = Description of Artificial Sequence: Synthetic oligonucleotide
  source 1..18: mol_type = other RNA
aggagccatgttcctttc

SEQ ID NO: 221 - RNA, 18 nt, synthetic construct
  misc_feature 1..18: note = Description of Artificial Sequence: Synthetic oligonucleotide
  source 1..18: mol_type = other RNA
tcaggagccatgttcctt

SEQ ID NO: 222 - RNA, 18 nt, synthetic construct
  misc_feature 1..18: note = Description of Artificial Sequence: Synthetic oligonucleotide
  source 1..18: mol_type = other RNA
cctcaggagccatgttcc

SEQ ID NO: 223 - RNA, 18 nt, synthetic construct
  misc_feature 1..18: note = Description of Artificial Sequence: Synthetic oligonucleotide
  source 1..18: mol_type = other RNA
cgcctcaggagccatgtt

SEQ ID NO: 224 - RNA, 18 nt, synthetic construct
  misc_feature 1..18: note = Description of Artificial Sequence: Synthetic oligonucleotide
  source 1..18: mol_type = other RNA
tgcgcctcaggagccatg

SEQ ID NO: 225 - RNA, 18 nt, synthetic construct
  misc_feature 1..18: note = Description of Artificial Sequence: Synthetic oligonucleotide
  source 1..18: mol_type = other RNA
tgtgcgcctcaggagcca

SEQ ID NO: 226 - RNA, 18 nt, synthetic construct
  misc_feature 1..18: note = Description of Artificial Sequence: Synthetic oligonucleotide
  source 1..18: mol_type = other RNA
ctgtgcgcctcaggagcc

SEQ ID NO: 227 - RNA, 33 nt, synthetic construct
  misc_feature 1..33: note = Description of Artificial Sequence: Synthetic oligonucleotide
  misc_difference 1..3: note = This region may encompass at least 3 nucleotides
  misc_difference 4..10: note = This region may encompass 1-7 nucleotides
  misc_difference 11..13: note = This region may encompass at least 3 nucleotides
  misc_difference 14..20: note = a, c, g, or u
  misc_difference 14..20: note = This region may encompass 1-7 nucleotides
  misc_difference 21..23: note = This region may encompass at least 3 nucleotides
  misc_difference 24..30: note = a, c, g, or u
  misc_feature 24..30: note = This region may encompass 1-7 nucleotides
  misc_difference 31..33: note = This region may encompass at least 3 nucleotides
  misc_feature 1..33: note = See specification as filed for detailed description of substitutions and preferred embodiments
  source 1..33: mol_type = other RNA
gggnnnnnnngggnnnnnnngggnnnnnnnggg

SEQ ID NO: 228 - RNA, 28 nt, synthetic construct
  misc_feature 1..28: note = Description of Artificial Sequence: Synthetic oligonucleotide
  source 1..28: mol_type = other RNA
gggagccgggctggggctcacacggggg

SEQ ID NO: 229 - RNA, 11 nt, synthetic construct
  misc_feature 1..11: note = Description of Artificial Sequence: Synthetic oligonucleotide
  source 1..11: mol_type = other RNA
aatttttggag

SEQ ID NO: 230 - RNA, 11 nt, Homo sapiens
  source 1..11: mol_type = other RNA
aatttgtctag

SEQ ID NO: 231 - DNA, 32765 bp, Homo sapiens
  source 1..32765: mol_type = genomic DNA
aggcggcggcgggcgccgggaagaaaggaacatggctcctgaggcgcacagcgccgagcgcggcgccgcgcacccgcgcgccggacgccagtgaccgcgatggtgaactccagtcgcgtgcagcctcagcagcccggggacgccaagcggccgcccgcgccccgcgcgccggacccgggccggctgatggctggctgcgcggccgtgggcgccagcctcgccgccccgggcggcctctgcgagcagcggggcctggagatcgagatgcagcgcatccggcaggcggccgcgcgggaccccccggccggagccgcggcctccccttctcctccgctctcgtcgtgctcccggcaggcgtggagccgcgataaccccggcttcgaggccgaggaggaggaggaggaggtggaaggggaagaaggcggaatggtggtggagatggacgtagagtggcgcccgggcagccggaggtcggccgcctcctcggccgtgagctccgtgggcgcgcggagccgggggcttgggggctaccacggcgcgggccacccgagcgggaggcggcgccggcgagaggaccagggcccgccgtgccccagcccagtcggcggcggggacccgctgcatcgccacctccccctggaagggcagccgccccgagtggcctgggcggagaggctggttcgcgggctgcgaggtaagagcgcgcgacccgcagcggcagatgcacgaaccagaacggccggcgccggccggggccatcgcccgctgcggcagctccccgggctccatctcgcatcccctctgcgttccgcctcccttggaagcgcattccccacctccgctagtgctgccctatttccggtacccagcgcggaattccactgctcttttgttggtgcatatttattggatacctccttcttcaggatatgtcaccatagtcttttttactgaaaattagtgaaagcctaattagagtgaaagagtacatctgggttttgtttttttttttcttgtagaggaaaaaatgaacattacttgtgtaactgatggtagttgcaactgcatatttgccaatgtcacaaaatctaaaggaaaatgttatagtcacccgtggtttccttcttgcctggacactccattgtcccgggctgaaaagggtagcagtacagtgcatataatgtcaagttgtgggaggagtgtggcagattgtcattggtgcatttttttggtgatgtgtgtggttgttttgaggagtgggagctgttaagaacaccacaaatagaataaaataatatcgtgaagttatttggccgtttctaattctagacatttttctaaaaacagttgcaaaggaaagattacattgttttaaaaaaatttgaagtatgtttttaaataactaaattaatgttctttgaaattccaccaaaatgatgaagtcaccagatagcagctgataaatgtgtctgagcccagtgcgcccagctctacaaaaggcagaagaggaattttcaaatttgccagtgcccagtaagaggacatgaattttctagtaccagaggaaattctctttttacaaattttgtcagaggtatccttgggaaagtatttcatttgcttttaccctccaaattattttaatctatatttttaagagtttccattctcagttgagtttttcttgttcttctccttctgtcagttttgaaagtcttgcacaaaaacaatccaggtgttgttacagcagtgtgattaaaaccaggtcaggcctacactgaaatcccagttccaccactcattagctgtacgaccttgagcaagttacttatcctctctgatacccaatttttggattttttttttttttaaagagatgatcataacgcttaggacttgactctttgggagaattaagtgagttaagacataaaatgtgcagcatgtatttgtcatgttattagcgcttcagaaatataaatgtaaacattttggtacttcgtttatggagattcttatactagttaattttattaaaagcatgatggggaacagaagatccttttcggataacctgtgtgagtaaattaataaaacactaacactttttaagattcaaaactggattaattatgttattttacaccatttaaaatgtgcatttaaaaaatattcactgaagtgagagagaagttccttttaagttaataaataatggtaagttgcatgatccttttaactcagtttaagcatttgataacacccctaaccttgtttgaataaactcaataacagaatatagagaaataaaatatatatttcatatgagtatgtatgtaaatttatttcttttaaaggaaaatttcaggaaacaaaatgaataagctcatagtcataaaacctctagccaaagtgtgtcaatggtctgatttgtaggagagcagactcaggaatcgggaattttttttttttttttaagagtcggggtttcactctgtcactgaggctggagtgcggggaagcaaacgtagctcactgcggcctcaaactcctgggctcaagctgccctcccacctcagcttcccaagtagttgggattacaggtgcaagctgctgctcctagctgaaaagttttaattatataaaatgtttaaataaattgttattcctttcttttatgaagaaatataattgatattcttgtcacttattaaataagcaacattttaaaatgtttagcacctactgtgaagatagcactgtgctagctgctatgaataaactagaatagcatatcactgttttcctatggacaaaaacccagaaatgtaaaataaatagccatgaatgggttgagattccccctgccccctttggatactgatgacagaaatcttatatgcactatgtaagcagtgccctaggatcagaacagaaagtaacttgcaatttttaaaggtgggagaaatgactgaagtttggagtggtcaggagtttccctggaagtctcttgcctttgtgactttgtatagctccctcaaactacctgagcctgagctcctcatttataaaatggaagaattgaacctagcttccaaggtcctttctagcttcaccatgctctggtctgttgtttaatgacatgaatccaagatggacaacaaatggtcgattttgctccttctacagtaaatagatctatcttcttagcagaagtaaaatagtaaagaaagaagacatgtttgaggcctgttgaggcctgtttgctgtatgcattgtatctaatcaaggaattgagaatgtagccctaaatattaggaaggagttgaaagtttctgagcaggaacagaacatgctgaaaggaaattgtgtcagcagcatggtgtacaggagagcttggcagagagaaaggaggcaagaagacctgtgggaggcagccagtaaggaggtgaagagggcctggaccaaaggaagccaaggatgaccgaaagatttaaagatgaagcccagatttaaaagtatcctcaagtatttgtttattcttttcttagcaaattctttttagtacaaagataaaatatggcactctgagtcataaaattttctgaatttcagaagttgaatgtttttctgaatgtatcaacctgttaaagtcagttcctgtttgttattttaggcgtatattcttgggcttttttttttttttcctagagaaactatgaagtactagctgtgcaagtcagggtgggctaagctgctaaaacagatacctccctccccttcaataccctgaagaacaagaagttaattttttgctcgtgtaacattttagagagggtattccaggttgaatacctggaataatgaatgaaataataagtcgactcttatttctcctaaaataattggatcttgacattgaaattggtgtgctgattgtatttaataagtgacctccaatgcagttatttcattttgccatactttatgtaatttttattttttctgccttccctttcgatgtctaaagggaagcataattagttattggaaaggttattgagacttaaaaaaagatttgaacatggctttggttgatgctaccacacagaaactgcaaacatccaactcaccgctcttagttgctgttcatgatagtctatgcacagtgactgatgaatttagcctgtcttgaggcttcagaacttagtcttatgtcatcacgagcagagctttatcctaaattacaggttttcaatgggggggggggggaggggcagttttgcccctcaggggacatttggcaatctctggagacatttttgattgtcatacctggggggatgcccctggcatctcctgggttgaggttaaggatgctgctaatctacaatgcacagtacagccaccccacacacacacactacaaagcattctcccacccaaattgccaggagtgaggactttgagaaaccctgccctagactgttaaattcaaaaggaaataatggtttattgctcacagtgtgccaggcactgtgttaaactccttatattcatagttttgtttttatcctcacaacaacctgtgaagaaagaactctcatccgtcaccactttactgttgagggcactaagccttctaaaggttaaatgacttgcccagggccgcaactattagatgttgcagtcaggatttaattccaggcactttgtgttcaaagtgtttctcatccactgtgctatatgccagtagtgcccaaacctaactttagccagcaattgtctgcatctcttcagtttatgaacatttatttattaggacatgcaggataatcataccaacacagtccgtgtatccagaattctaattgatctagggagtgggagagcctgcctctctattttttttaaattggtgtgaaatatacataacataaaattggccattttaaccattttaagtatataattgagtgacattaggtagatttattatattatgtaaatggacttaacacaatttatttccagaacttttttatcatcccaaacagaaactctgtactcattaaacagtaactccctgtttccccactcccccttgccccagacgctagtaacctccattctacttcccctttctgtgagtctccctgttgtagctacctgatgtaagtgaaatcagaccatgtttgatcttttgtgtctggcttattcacttagcatagtattttcaaggtttatccatgcgtgcaaattcccttcttttttatgacgaaatactatttaattgtgtgtgtacatgtgcacacacaatgaaatactatttcatcataaaaaggaagtttgtacacatatatacacacacaccaaattttgtttatccattcatcagttcatgggcatttgcggtttttttccacctttcgactgttgtgactttccgtttatttttaatctgaactttactccatcacttcctccctttccttttttattcgcaccataattttgaacaagcagactttgtattttcattactctggggatttttttgagggaggcttgctaccctttggggttgtggtaaggttgtgccagtaacagaattcatgcagtaaaaaacattcatgggactttcttttgtgataggtactagggatgcagagatgaataatacaaggtctcgaccttcaaggagctcagggtttagtagggaaaacagaggtataagtaagtcattgcaatgccaggtcccagatatgggaggattccagcaaagagaggataaggccaggcacagccgctcatgcctatagtcccaacactttgggaagctgagatgggaggatcacttaagcccagcagttcaaggctaccctgggcaacatagtgagtggcaaaaaatacaaaaattagccaggcagggtggcacatgcctgtggtcccagttatttgggagtttgaggtgggagaatcacctgagcctgggagattgaggctgcagtgagctgtgatcacgccactgcactccagcctgggtgacacagtgagaggtgagagcctgtatctaaaaaaaaaaaaaagagaggatcagatttacctggagaggtcagagaaggtttccagagggagtaaaacttgagcttcattttgaagaatgagaggggaatacaaggagggaaaaataataggaacaagacctgttcctgttacagcaaacctgtaactcccaccatggaatgggcacgttttcctgtagacatttggagccagtgtgggcttcaagcctagggggctgatatgatcatttgtcaatctgaagatgactcttggaacaacgtagaggatagggtgaggtgggtaggctggtggctgacagactaggtcaggagagggctgaccaagaggaggaggggtgacgagggctggaactaaggcatatgagctgggatggaaagaagctagatagaaaggaaacaaaaaccatcagaactgtggaaccaactccatgggagggataagaagagggcctattctagggtgaaacccaaatttctggcatggtaccattaacccaaacagaaagaggagatttccagagaaagaaaagcaaattttgcgggaggtgagtgtgagctgtctagagaatatgcaggtagaactacatgttaactgtaacatatacttgtctgcacctcaggagatggctgattaagaattcaggattcaggccaggcacggtggcacacgcctttaatcccagctgcttaagagactggggcgggaggattgcttgagcctgggaatccgagatcagcctgggcaacatagcaagacactgtctctaaaaaaaaaaaaaaattcaagattctggagtcaatattacttaaggtagcaactgtctattctatagaaaatggacaaatatagataaaaagccacttccctttctaaaagtgtccctgcaattcaagtgaatactaggaaggattgtgttcattcttctaaacaagactcacatgtatcttagcacaaaaagaggattcttttatgatacaaatgcactgagaatttggtcaggctatcacaatgaactgatagttcagatggatttgagtctttataccactctggaatctggaccaactgggcctcctaaggccattttgcagatctgggcttgtttctgaagctacagacaggcctcttccaagcactctaagtgctccacaaataatatttctgtttcccagaacaaccccacaaaaaggtactcttactccatttttagatgaggaagtggaggctcatgatgtcaggtaagctttctcagctcccaagtggttaagccctcagtttaatgtcatttgactccagagccctatgttgcaccatgccttgataataggccatatgggtttcatgtatttcagatggggaaggttagtgtgaggtgaaagatacacaattaaccttttaaccatggaactgaaatatttacagatgaagtgatacaatagctggaattaattccaaaataattggggtggtactggtccatggcctgttaggaactgggccgcagagcaggaggtgagcagcaggctagtgagcattgctaccccctatcatatcagcagtggcatcagatgggtgaggatgtaaatgaactaggattggcattgagttgatattgttggatctaggtgatagatttatcattataatattctctctttgtgatatttctgatattttctaaaataaaaagttgtagttatttatttatttagacggagtttcactcttttgcccaggttggagtgcagtggtgtgatctcggctcactgtagcctccgcctcccgggttcaagcgattctcctgcctcagcctcccgagtagctgggactacagcctcccgagtagctcaccaccacacccagctaacttttgtatttttagtagagaggaggtttcaccatgttagataggctggtctcaaactcctgacctcaggtgatccacccacctcggcctcccagagtgctgggattacaggcgtgagccagtgtgcccagccaataaaaagtttttgaaaggatttagataaaatagttggggaaatggcatttttgttcaaagccaattatttatgtttggaatatcttttgtgcttggagttctccattacagagttccccagtgttcctattaataagtaacattgagcagaggaatgcactgtttagatcagcagtccccaacctttttggcaccagggaccggcttcatggaagacaatttttccacagacctggggtaggaagtgtttggggatgaaactgttccacctcagatcatcaggcattagtgagattctcataaggagcaggcaatctagattcttcacatgcgcagttcacaatagggttctcactcctgtgagaatctaatgccacccctgatctgacaggaggcggagctcgggcggtaatgctcacttgcctgcggctcacctcctgctgtggggcccggttcctaacaggccatggaccagtacccgtctgcagcctggggactggggaccctgctttagatgatgtactctggctttgcattctggcattagctaagcaccctcttaaaggaaattgggtctatactctcagtccgtgttctccctaacacctggaaacattgaataccttcaatgctgggaagttaactcccaccacaactagaacagctatgggaaagacaacagttgattttgaagagtgtcaccaatttgcacatgattccatccttaaccattcttatcctatcagctctgccaaacatggagaatagttggctgcaggacagctatttttcctacttgtagatgcaactatttctcacccaccaggatgtaaaaggtccctgtaccctaagattggtcctacatacacacccaatgggaaaatgagatgaaaaatttaaagcagtaaatatttgaggaagtagatagagtaatttagaaaaagaaaatacacagggccaagcacagtggctcacatctgtaatcccagcactttgagaggccaaggtgagaggattgcttgagctcaggagtttgaggccagcctaggcaatgtagtgagaccccacctctacaaaaaattaaaaacttagttgggcttggtagtatgtacctgtagtttaagaaacttgggaggctgagctgaggcaggaggattgtttgagcccgggtggtcaaggctgcagtgggccatgattgtgccagagtactccagcctgggtgatagagtgagactctgtctcaaacaaaaaaaaaaacagagacagaaaaaaagaaagaaaatatatggatgtatatcatataaaaatataaataagggaggccaagtgcagtggcatgcctgtaatcccagcactttgggaggctgaagcaggaggatcacttgaggccgagaattcgagaccagcctgggcaacgtattgagacctcatctctgcaaaaaatcaaaaaatgaggcggaaggatggcttgagcccaggagatcaagccttcagtgagctgtgatcgtaccactacactccaggctgggtaacagagagagaccctgcctcaagataaataaattcatacatacatacatacatacgtacatacatacatacataagaagacttgtttctttccatttgcaatgtttcattcaaaggctagaattaaattgccgtaggccatcacaagtttagcttgaatattattattttttcaagatggagtctcactctgtcacccaggctggagtgcagtggtgtgattttggctcactgcaacctctgtctcccgggttcaaccgattcttctgcctcagcctcccatgtagctgggattacaggcgcccgccaccacacctggctaagttttgtatttttagtagagacagggtttcaccatgttggccagactggtcttgaactcctgacctcaagtgatctacccgcctcaccctcccaaagtgctgggattataggcttgagccactgcacccagcctagcttgaagaaaatttgataggagtttgtttttttctatttataggccaagcaataccacgtataaatattaagaatcatggctgttccttagtgcctagttgtttataaaccatgggaaagaatgaaatcattgaccaaatgagacagggtaacagtcttccctgggagtaaagagactagcagtctctattgacatatattttaggcctggcctccaaaataaatttacccaaagaagtgatgtatttgtgtccagggctaccgagcctaatctttggctgcctctgtgttattctaagttgtaattttccatgtcatctaaatttgtaataattctttaatatgatagtgtttcagtgaacaaacattctgcttgctgcatttcttctgagtgagtaattccctgacaactaccagatcttggcagaagcaaagttggtcataagttatgctccactctcagtgctggtgaaacgtatgatgcgtaacacagtgtttttgaattcagtgctgattttcctaaaggacatttggaaggaaaaaagaaaaggaaaggaaatacccaaattcaggaatagaacttacatattattataagacttaaaaaatacatgaacactaatgatgaactcatttcttcgaagtaaaaggccttatgctattttttcccatttccctatgtggcttgattgtggcgaaagtggctgtgtgagtttccattattgaaggagttaaggtctgtggaatcaaagcatgagacaacatgcaaggaccaggttggtttccatttaacagccaacctaatctaactgaaaggattgagaggtttgtcttttttggaaagtgttaaggttcttccaagtaaccagcaatgtgactttaccacactgttcattaacggggctttggatggccaccatgtctgctgctggctgagtccaaaactgcggtcactctctccagcaagctcatggagggttacgtcatcttcactcagccagcccaacgctttttccatctgctaaaatgtagaacatggttgttgattctactctttctacaaagaaagagagggaacatggatgagttgctgcttttaaagattatatgttaattgctgttttaaaaatctgctcagctaagcacgcttagtgtaatcagtcaccatggagttttttaataggacagcttctgctcttacagagcagagtttttatgccagtaggttagaagcataacatgtttctatattgaagtgattctccaacaaggacttcttttcatcaggggtgacctaaaagttattacttgaaatataattacatcatgttcttaactgggtttagtagtaggataattacaaggttgtatccacctcaaataggaagaactgataagttttgcacaattatttaactcctctgttaggccttccttatttcctgttctctttttaaaatttgactaagatattgctaatgggcttgggagcctataaaatgatcagaattgttgccttatgttttgcatgtttggggtaacattggagccagatgtactcttaaataaaaggtaggcctacaaaaccagtttctcagttgcattcaaaatgtgattaaaaaaaaaaaaaggagaatctcccttataggtgaactttttaatttgtgctttattttcccttttgccattcatgagatttcttaaataaaatgatatcttttcttttcttcattattattttaaaggtctctggggaacaagactcatggaggaaagcagcactaaccgagagaaataccttaaaagtgttttacgggaactggtcacatacctcctttttctcatagtcttgtgcatctgtaagtagaatatttccttgcactaatgggaaagttttgaaaagatttgacctatccaaatcataattaaaaggaagtgtgtatgcaccagaggggcaactgggaagttaccttcttacctttgtttttaattctaatatttttatttgggcatttgtttattgactatcttcctatggtagaatgcaagctttataagagaagggacgtgatttgttctctgctgtacccccatttcccaaaactgcagatggcaacagaaggctctgaaaaatatataagaaagaatttttctaattgtgactaaattgtgaccaaatgctaagtgactgtggacttgcgtttaacacaggacgggagaggcaaagagttcaattccaatttagaatttggtcaagttctcttctgcactctggtaaacattaattaaaaatcagcattatctgaccagccagttcatcgtcagtggtggtgattttcactatgagatacgcgtggcaacttgccagacaccaagaaaccaagttagaggatttttgtattagattccttaacaatgaatacagtatcaccattattacagtatcatcattattgtcatactattattatatcagttaacataaagtctgcataagaattgtttccagaaaaatgactttccaaatttaactttcaggaaatacaaataatgctactaatattgcttttattggcgtatacatgtaatatccccttcttttggatttggatatgttgtgtcattgcctcattttaattcattatttcttctcaatctttaataattgctggacttttactccacaagaaacttgctataggcccatctctttcgtcttctttccttctttcagttcgtcttcccatcctctggtagggggaggggagggatgcctgagcgagagactagctgtaggaaccatttgtctcaaagtccagaaagccacaggtgatggatttgtcctctgaatcaaagggcgttcgatgatggatttctgtcatgtctcatctaaagtcttcacgagaacagatgaggaagcagttttatgaccccagagcctcctaccaaactcctctgagaaaaggtttccttttttttttttttttttaaattagagacagagtcttgatctgtttcacaggccggagtgcagtggcacaatcatagctcactgcagcctcgagctcctaggcttcagtgatcctcccacctcagtcacccacgtagctgggactacagctgcacaccaccatgcccagctaacttttaaaacatttttgtagaggtggggtctcactttgctgcctatactggtctccagctcctagcttcaagtgatcctcctgccttggaagttctgggattataggcatgagccactgcacccagcctggatgtgatatttttatgttttaaattgttagagtttagaaacttgagattgagtttgctgcctgcattaaaatgatgcttaaacattaaactgcagtggccttaaatattaacaagttgattagaattactaagttcttttcaagctttacatatacagacaaatttcttatgcaaaatagaaggtaacccctgtacgtaagtctagaatttcagcagtccccaaaactgactgagcattagaatcactctgattttaaaatacatatgtggttttccgagatctactaacagagcctccataatgtagcctagagaaacgagttttcagatgtagtgataaatttggaaggtaagtcagagaagtaagctgaagacagagttttaggaaatatgcctaaagtcacataatgaattggttttcttgtttatttgagaatattgtcgctttttgttctttttcatgcaaatcacattttatttcttatgtgagtagctatatatttaaaaattttgtttttggaatattgtagaatctctacttaagaaagtatcttagcagtcatatggtctgacctcactgaatgctaaattctctttaaaacatccgtctcagatggttactcatactgcctctgattgaacaagagtcccaggttaagggacttacttctttgaaatcgttcatttcatttttctacagctatgttagaaagttcgtccttagcagtgaagccagagtctatctcttataacttctacccagttgcaccctccaagcctacctataccaagtatcttttttccacgttgcttttccaattcagctcttcgcaagtttggagacaaatacctaatctcctctaagccttctccaggttaagcctttccagttcatccagctgttgattatgtgattggagacacaagtttgagtaactcccatgatggaaagtccctctggtgaatgttctgttcatcagagtccctaagaaagcacatgagcctcaccatggtgagtggggccatgagattcctaaaccagacactacactgtggcttgtgcaacctaccatggccagactcatgaggctgtttcatcatataaataattccttagtctcttcaccaaaaactgtgaagcactgtgtcccccagctgtatgtgagctagcctgggaccatagtggaggactcctctttcaaccctgttaaatttcatcttgttaggatctgcccatttttccattctgttgaaattatcttggaactggattcattcatctcacatcagctactccccgaagcctcacagtatcagcagagttattatctctatccatatccttgtaaattattaaactaaaaaagattggtctaagaacatcagtccattaatcaaggctctttggttggagttcacttcattatattatcatccagctcagagtgttcataaacaagatgataggaaagacttttccaaatgcctgtctgagtaattcccccattcctgtgatctgtgagctgttgaccagattaaaaaggaagtgagaataaccaggcaagatttactcctagcaaaaccttactggcttctagtgactgagtcctttccctattgcttaccagctatctttttaattactagttttaaaatcttgccagcaatgtcaatatcaaatgaccaagaatatcaaactcattactctgtatgtaaatgagatagtgtacttacccctatggcttaagtattaggctgttcttgcattgccctaaatacctgagactgggtaatttataaaaaaagaggtttggccaggcactgtggatcaggcctgtaatctcagcactttgtcaggctgaagcaggtgtaatggtgagccaagagttcaaacttagcctggacaacaaggtgaaaccccctctctgcaaaaaatacaaaaattatctgggcatggtggcatgcacctgtagtcccagccacccaggaggctgaggtgggaaaattgtttgaagctgggagtcagtgattgcagtgagccatgattgcaacactgcactccatccagcctgggcgacagagcaagaccctgtctcaaaaaataaataaatgaataaataaataaaataaataaataaatagaaaagaaaaagaaagaaaagagatttatttgcctcatggttctgcaggctgtaagggaagcatagctccagcatctgcttctggggaggcctcaggaagctgttactcatggcagaaggtgaagcaggagcttgcacatcatgtggcaaaagcaggagcaagagagagagaatggggcagggaagaagccccacacttttaaatgaccagatcgcatgagaaatcactcgttacctcaaggacagtaccaagaggatggtactaaattcctgagaaatccacccccatgatctgatcacctcgtaccaggccccgccttcagcattggggattatgtttcaacatgagatttggatggggacaacatccaaactatatcaccttgcatagtaggtagggttttaaaaagcagtttggcacagtaagaaaagtacagattttttttgcatcagacagacctgagttaaaatcccagcttcactgctaacatgctaggtaaatgtgggcaagttaattaacatttctaagcctttgtttcctcactggtaaaacaagtatttggaaatatcattgtgaagattagaaataatacatgaaaagatcctaggatgctgtctgtcatacagtagtagtagtaagaagttattcttgccaaagattgttgagaatggcagaattatctcagttctaagagctatagtttctaattatttgagcctagactcagattcatttggagcagctaactgctcaccaagagcttattttccatcttaccaatgaggttatgtgccctgtgtttttaaaatcagtctacttaaccaagagaacagaaatgacatgagaattaagtaatctcactttctctgttatttaggatttattcctactcaaaacctgagagttgctatgaattcaccattaaagcacttattaatatacatgggttactgttataaatagcaatagtattgctattgtgtgagttaggtgttgaagttcaagaaaggaataaagaatatttagaagatctttgaaaacagtgtctgggtacggtggctcatgcctgtaatctcagcactttgggaggccgaggcaggcagatcacttgaggtcacgagttcaagaccagcctgggcaacttggcgagacctcgtctctacaagatatacaaaaattagccgggtatgttggcatgcacctgtaatcccagctacttaggaggctgaagcacaagaatcacttgaacctgggaagcagaggttgcagtgagccaagattgtaccactgcactccagcctgggcaatagagcaacactctgtctcgaaaaaaaaaaaaaaaaaaaaaaaaagaaagaaagaaggaaggaaggaaagaaaaaaaaaggaaaaaatgcaaggaaggtatttggtgaatctataataataaaaatgtatttgtcatttcctttttctgtgctctcattctataaaattgagtaaaaaatctatatatagtttaaacacattaatagaaatcacaaaagttagctgagtcaacattgtagaaacataatatttctgtatgtcaaagaaatagacaacattaaaaagcagaaagcaattacaaagaatgattataagaaacatcacaaagggttaatattttaacacatttgaaactcaaaaatcactgagaaaagcagtagacttccataaatattttatagagtagaaaaaataggccaagcacagtggctcatgcctgtaattccagcactttgggaggccgaggagggtggatcacgaggtcaggagttcaagaccagcccggccaagatggtgaaaccccatctctactaaaaatacaaaaactagccaggcgtggtggcaggtgcctgtaatcccagctacttgggaggctgaggcagggaattgcttaaacccttaaacccgggaggtggaggttgcagtgagccaagttcgcaccactgcattccagcctgggcgacagaacgagactctgtctcagaaaaagaaaagaaaagaatagaaaaagaatccatgggcaggcacagtggctcatgcttataatcccagtactctaggaagccaaggtgagaggatcaattgaggccaggagttcaaggccagcctgggcaacatagcaagactttgtctctattaaaaattttaaaattagccaggcatggtgacgcacacctgtagtcccaattacttgggagcctgaggcaggagaactgcttgaggctgcagtgagctatgattagaccactgcactccagcctgagctacacagtgagaccttgtgtcaaaaaagtaaaaaaataaaaattagccaggcatggtggcacatgcctgtagtcccagctactcaggaggctgaggcaagaggatgacttgagtctggaagatggagactgcagtgagctgtggtcatgccactgcactccagcctgggtgacagagcaagaccctgtctcaaaaaaaaaaaaaagaaaagaaaagaaaaataaataaaatttattcaaatacaaaagtgatgtggtttgactctgtgttgccacccagatctcatctccaattgtaatccccgtgtattgacggaggttcctggtaggagatgattggatcatggggatggtttcccctctgctgttctcatgatagtgagtgagttctcatgaaatctggttgtttggtaggtgtctgtcacttaccccttctttttctctctcctgctgccttgtgaagaaggtacttccttctcctttgccttccaccatgattataagtttcctgaggccttcccagccatttggaactgtaagtcaattaaacctctttcctttataaattaccgagtctcaggcagttttttatagaagtgtgaaaatggtctaatacagagacttggtaccaggagtggggtactgctataaaaaataacctgaagatatggaagcgactctggaactgggtaacaggcagcaattggaacagtttggagggctcagaagaagacaggaagatgtgggaaagtttggaatttcctagagacttgttgaatggctttgaccaatacactgatagtgatatggacaatgaagtccaggctgagatggtctcaggtggagatgaggaacttattgggaactggagtaaacgtcactcttacatgttttagcgaagagactggcagcatttttcccctgccctagagatctgtggaactttgaacttgagagacatgatttagagtatctggcagaagatatttctaagcaccaaagcattcgagaggtgacctggcttttcctgaaagcatacagttatatgtgctcacaaagagatggtttgaaattggaacttatgtttaaaggggaagcagagtgcaacaaaagtttagggagtttgcagcctgaccatgtggtagaaaagaaaaacccattttctggggagaaattcaagctggctggagaaatttgcataagtaacgaggagctgaatgtgagttgccaagacaatggggtaaatgtctccagggcgtttcagaaaatcttcagggcagaccctcacaacacaagcctggaggcctagaagggaaaaatggtgtgagccaggcccaggcccaggccccagctgttctgtgcagccttgggacatggcaccctgtgttccagccactccagctccagctgtggttaaaaggagccaaggtacagctggaccattgcttcagagggtacaaatcccaagcattagcagcttccatgtggtgttgggtctttgggtgcacagaagacaaaagttgagctttggaagccgctgcctagatttcagaggatgtatggaaacacctcgatgtccaggcagaagtctgctgcaggggcagagccttatggagaacctctgctagggcaatgcaggggggaaatgtggggttggagctcccacacagagtccccactggggcactgcctcatggagctgtgagaaaaggaccaccatcctccagactccagaatggtagatccaccaacagattgcactctgcgcttagaaaagctgcaggcactcaatgccagcctgtgaaagcagctgcaggggctgtaccctgcagagccacagaggtggagctgtccaaggccatgggagcccaccccttgcattagcatggagacaggggatcaaaggagattttggagatctaagatttaatgaatgccctgtcgagtttcagacttgaatggggcctgtgacccctttgttttggccaatttctcctatttggaatgggaacatatacccaatgcctgtacccccattgtatcttggaagtaactaacttgcttttgattttacagactcaggcagaagggacttgccttgtctcagatgagactttggacttgaacttttgagttaatgttggaacgaattaagacattggggttctgttgggaaggcgtatttggttttgaaatgtgagaaggacatgagattttggaggggccaggggtagaatgatatggtttgactctgtgtctccacccaaatctcatctccaattgtaatccccatgtgtcaagggagggacctgatgggaggtgactgaatcataggggcagtttcccccatgctgtttgcatgatagtgagggagttctcatgagatctggttttttggtaagtgtctgggcttcccccttttccctctctctcctactgccttgtgaagaaggtacttgcttctcctttgccttctgccatgattgtaagtttcctgaggtctccccagccattcagaactgtgagtcaattaaacctcttcctgcctattctcaggcagttctttatagcagtatgaaaatggactactacagaaagtgtgtaactttaaactcagtagtatccaaagaagtaatgaaaatggagaaacgaacaacaaaatcatagtacaatatggtgtatgtactaggacaggaagagcccttttaagaagagatctatgtatttccatttgtttatctctgaaagaaagcaactttgccttgtattctgaaaaagaaaggaatattttattttacttgtaaaaatcttacaaggatgctagtctaaatatagttttcctaatttgccagagaatccatgaagatcgagttgataacaagatcagtgaagtaaaggtcagtgagttaatctcacagcagctgcaggctaattccatttccagtgaaaaacgtcttgattgctcaccacatatcttttcaccacaaacagtttcagtcttaagatcacatgttgcaatccatgagaagtaactattaagccttcaactatgactggagggctcctcgccctttctgataaattgactggacaaaaactcaattttaaaatgacaagaaatagaagatgtataaatgtactttaaatgtgaccaaaatgggttgtgaaaacacaagacacaatatccaaaaatgctggcaacacagtacactgtagagtattggttgtttatttacccttgctattgtgtggctgagcttactgccactgcccagcattgcaagggcatcaaactgcctatcactagcctaggaaaagatcaaaattcaaaattctaagtacagtttctactgaatgcttatcacttttgcaccattttaaagtaaaaaaatcagtaagttgaaccatcatatatccaagattgtctgtatataaatattatacatctttctctcacttttaaaacaaaataatactagccaatactaccattctcaaaagcacttgtgtcaacagcctttaccccttaaagattttcctcacaattttaaaattgttacttactattttctttgaaatgttgaccaaacctggattaaaagatttgggggttttagtgactgtatttcacaaactctcttattgattctgcagcctcacttctgcctcctaaaaagccctcaccaaggtcacgggggatggctcttttcagcctcttcctggcatttggtccagtggcatttggcattctaggacttccctctttgtctttgataactccctctcttcctgtgttcctccttgctgtgttcacttgcttcgctttcttctttctgaagcatgtttacacagtgttttctctgattgggcctgtgacgttctttaggtcatcttttccacaaataatgcttcaactagtacttgcgtgccagtgactccacgtcccactcatgagctctgaacctagtaccagcttctgctggacatctacaatgggatctctcacaggcctctctcattggtaacatgccccagcctgaactcatctcccacccatctatccagccatgctctctagttcacctgaacacttgggtgtcatcctagatgctttcccttcccagtcttctgtgatcattctgcctcatcagaggctctctaatctgtcttctttcctatatcgctcttgtccctattttaatcctaatcatctatttcctgacttattcattccttaagttggtcagtaatttaattaaaaacagatttaggccctgaccttaaatgtgataagtgatatgaaaggagatgactggggaaaaggatttccctcaaggaaggcctctgtgaagcctgaagcaagaatgaaaacgagtcagacgaagagagaattgtatgaatgaaggctctgaggcaggaaaacactcagatcattccagaatcacttagaagccaagtgaagccagttcctggagagcagatcatcaaatgaagatggaaaggtgaccaggggccagacctgtagttttggtgggccttggtgagggatttacagtaggacaccccatggtttaagtatgaaagtgacaagattcctttaagttttaagaggcctcgaaatatgaaccacagattagatggaagctactctccctgtgtctggactttttagaatttccaagagctgctgtttctggaaccagattaatacaagtcagtcttccatttatttatttatgtatttatttgagacagggtctcactctgtcacccaggctggggtgcagtggcatgaacacagctcactgcagcttgggggctcaagagatcctcctgcctcagcctcccatgtagttgggaccacaggcacctaccacccagctaattttatttgttgtagaaatgaggtctcattttgctgcccaggctgttcttgaactcttgggctcaagcgatcctcctgcaacatcttcccaaagtgctgggatcactcttccatttaacatgctatctcaacgtcaagataaactttaaaatctttagataataggctggcattttacttaaacgatctttacttcttcagaactgccattccctataaatatctggttcttcaaccacatcaaaccacttgtgatctcaaaaagcctcagcgtacactgtcctttctgtcattctaattcctcctcatccttcaaaatcaactcaaggaccagatccagggagaagcttagtggtgcccacccgaaccggccccctccttcgagttgtgctgccattcgggcccacctcttcacacagggttgtcagaccagaccagctcatgcgttcaccgcccttgcagggatgggatgcagctgtgcacctctcagtgccgacacctggagagtctcacgaaatgttgacaacatggcctgtttccatttcttgttcactaggacttctcatttactaacacacagaatttcctgtagtatgtccacttaatcagttcaagcctaataattccttgatttgggtatagtgctttgcatttatatactgatggtccccaacttacgatggttcgatttatgatttttcaacttcatggtgatgtgaaagtgatacacattctatagaaaccacactttcaattttgaattttggtctttttccaggctaccatactctaaagatagagccacagatcccagtcagccatgtgattatgagggtaagcgaccaatactctacagtgtattgtattgccagatggttttgcccaactagcctaatgtaagtattctaaacatgtttaaggtaggccaggctaagctgtgttgctcattcagtaggttaggtatattaaatgcattttcaacttatgatattttcaatttacaatgagtttatcaggatgtaactctactataagtcaaggatcatcttgtatagcacttttaatttatagagtcccttcaaatgtttgtttgtattttatttccactgcatccctgtgaggataccataagttatacagctaacaaaacagttagttttcctgtgcaaagtgatggcttcatcttgtggcagattacctggaatactgtggccaaggcatcttagttctactgtctttatatatctagtacagttatatttttatggcagctctgatttcttctttggcccaagggttattaagagagggaaaaaatttaatttcttaacagatatatatatctatgtcaagtcatatatttaattcaaacccttaatattcctaggtaatttttgtctactttctctgtcaaagattgaaagatacagggttttaagtttccaactgtaattgtagttttggtaattgttttattactaacaggtattgctctgtatattttgatgttctgttattcaatacataagaattcattacagttgaatcatcatgtatagtataatttaccaatgtaaaataacctttttatcaaactgagtattcaccttataatgtcctctgtcagaggattataataccacttaaaaccttttaaaaaatattttgttcctgataattttagctttgagtggttttcttataacaattctagagatgcgtttttattttttaaccaagatttaaagctttgtgatttagtaagagtctaaaccattcacagttcatgcttttttgtcaaatttctattatttagtattttcctctcttttatattttcctgttattttccatttctattcttttgttagactggaaattttttctttgctttcttttatcctagtgatttgaaatttatgtaatatatactattctacaataccctttatttgttttcaatatttgaacctatatttttcaacattattaagaataaaatagtatttgttgctattttgaaatgatagaccatgtttttaaggcaggttggtggttgttaaggcaccagtatcggccaggcacgatggctcacacctgtaatctcagcactttcggaggccgaggtgggcagatcgtttgagcccagcactttggccgatactgtggtttactgtattttgttctagtttattttatggaaaatgggaattcagtggttaagacaaggattaaatagcagaagaaagatgtgtacatatgtacagatgtatgtgtcctttatatgttttttagtactcttgtctcctttctggtcctcatttaaggttatctatttcatgcagtaaatttttcttcacaattcatttcatttagagagtgaatgctaccttccaagtgggctttctccagttttcctttcagggacttaaaggagaagtgatgttaacagttttatatttccattgcattttacagtgtgcagatgtcttcacatatatttccccatttgagctttacaaaagcccttagtattattctcattgtctagattccaaaatcaggcttagaggagttaaatagttgtccaggatctcaagatgcaagacccacaatcatgaacagaggcagatgttcaggatggaggcaagctgaaactcaaaaccaaatcattatgactccaaattcaggagtcttttagctgccacctgcatgggctcttggtgtagctgaccaccagagtttgtagagctgtcattcaggtgtgccatggactttcctgggacctggcacaggagaaggactgagttaatgtttgctgattaaatatctgttacaggctgggcgcggtggctcacgaccgtaatcccagcactttgggaggccgagcagggaggatcacttgagctcacaagtttgagaccagcctgggcagcatggcgaaaccccgtctctacaaaaaatttgaaaattagctggccatggtgatgcatgcctgtagtcccaggtactcaggaagctgaggtgggaggatcacatgagcccatgagattgaagctgcagtgagctgagatggtgccactgcactccagccttggccatagagccagaccttatctcaaaaaaaaaaaaaaaaagttacaataatcttcccttcaaagctggaaggcattatttacctgtctgtccagcagatggtgctacataaccaagggaatctgttgcttgcccttggtgaagctattaaagccaatacagatcttgagaatttcaaaagcaaaaatcaatactggattatgagtgctctaggaaaataaagagataaattttcaatttacatacttatatatagttataccatatttgtaaataaaaatatataaataatttatcaaaattcctttttaacagcaacaaccacagtaaacccacaggttaaaaactccacaacagtctatattaatcagtcaatgcaaagtacattccaattccaagttaactgaaaataatcaacttaatcatttggttggctctgagcagccttcactgcttgctcttgtgtcatgtttctttctgtcctcgattggctatagacttacagacggcttttgcagaggacagtgtactcatgtccatcctttgcatcctttgtgggagttggctaggcagcactctccctggggacactatgaactcctcttcttgagtgcagagatcacgtcttgttcatcttcatgcccaataccttgttccataaatatgaatggattagaattctaaactcttaactctgccccaagacagttctgagaggtagtaagtcatataacacctgaagaggactgttcttgtcctaattacattaggttataagatgacaggtgagggagccaaaccagggggcctggaaattattcatacatctctagatacagtatacaagttgtgtgtattatgtgtatttactctgtaattgattgcttgagatgaacccccaaacacactcgtgtttggatcattattatctacccttctccttaaataatcttaatttcctatgatgcttgaaagggaaagaggggccaggtgtggtggttcacacctgtaatcccagcactttgggaggctgaggtgggagcatcacctgaggtctggagttcaagaccaacctgaccaacatggtgaaaccctgtctctactaaaaataaaaaatcagctgggcatggtagcacatgcctgtaatcccagctacttgggaggctgaagtgggagaatcgcttgaacctgggagggggaggttgcagtgagccgagatcactccattgcactccagactgggcaacaacagtgaaactccgtctcaaaaaaaaaaaaaaaaaaaaggcagagtggggaagagagctgcatgaaggagagatttactaaatagtacttaatcccaaaataatttctataggtttgaatatgatccctgaaatttattataggttcaggtaagtattaatcacgggtattcagaactgtggtttaaaaaatgtatagaacatgtttccttccccttgaaactttttatcagctaattataggaattatattatacctgcaatcattaaagtccagaatgagacagtacttggtaaagtgctgaaatttataataaatgcattatagcaatccagttaaggaggaagagccaccattattgaacatttgttaagtgtcagccattgtactagataaattttagttattatttttatttaggcaccaaaaaatccatgggatagttggttatccccatcttactgaagaggaaactgaagctcagaaagtttaagcaacttgcacaggtcacatagcaagtaaggagcatggccaggaatcagaccctgatctcctttggtctactaagcttgcaaaggatcttcccgcctccttccaagaccattcaatattatcagtaaatgtccatggcaaggatgtagttcgagttatagggttccattcaagatatgattggtaggtgggaagcagatatgtctgtgtcaatcagtatcctggaagaaggagatgatgaactcaagtggtgattaagggaagtttaatgaagggactatttacagagatgtggtggggttaagagaaccaacaaggggaagtgatgcactcaaaaagttactacctccaggctttaggggattgggggagggagtggcacagtgtgaacccagtgtggttgtgagaaaagggattccctcagcagccatggccaaggttagagtctccactgccaaactgcatccaggtggtgagggaatggagaataggggtggggtaacaaactctgacctcggtatccccaaagggcaaaggattccaggtggtacagttcgtaaagattagcctcagggcacagaacagggcagagaagaatggagaattgatctggaggaaacaaacaatggcttgcccatgttattgcagggagagtaggctggtgtgcacagcaggagggtggggagcccagcatatagctgtgttggggcctgtgcagatcagcctcactggcagggaggatctgagccgagaggtggtggaagatgaaatcgagtaggcatgttggtagtcctaaatatcaagtaaacgttcctgatcttacattgatactcaatagtaagccaattttgtttcccataagccaatattaatattacgtatttcttttataagccagagatatagagagataccctagaagaatgataggggaaaggaaggcaagggtgagagaagaccttgtgtgaatttgtccaaaatgtttatccacaggaacaatccctttgtgaaggctgctggtatgtgaatgtgtgccggttcccttggggcgttcatttggatctttctgtgttccagtgacctacggcatgatgagctccaatgtgtactactacacccggatgatgtcacagctcttcctagacacccccgtgtccaaaacggagaaaactaactttaaaactctgtcttccatggaagacttctggaaggtatttgcaaataactttgaaagtacctctctatcacagaaaattgttcatttggcttcatcatttcaatgcatgagtatcgacaggacctgctttgcatttaacactgtgtgagacgtaagttatggtgagttgttagaagttactgttcctactctcaaagggggtaaactaacattgagaactttgcctgtgccttgcactgtgctgagtgtttcatatcttaccttatttaatttctatagtctaactctataaggtaagtactaagactatgccctagtttgttaatgaggaaaatgagattcaggatgtttaaatgcgtatggtcacatggctagggaacaagaaaaattgatttttttctagcctgacagctacttcatcctagtttgtaattcattccatgagtcaagattcaataaatatttattgagaatctcctagaatgtaaggccaatgaagggcagtgtggttcttctgtcttgcttcgccttttgtgttttgtctctttgttgatgatggcatgtatccccagctcttagaacagtgcttgattcaaagtaagcacattctttcaaaggtctgctgttggtggggcttggtggctcacgcctgtaatcccagcactttgggaggccaaggcaggaggattgctttagcccaggattttgaaaccagctgggcacaacatagtatgactttgtctctccaaaaaagttaaagaattagcagggtgtggtggtacacacctgcagtcccagctactcaggaggctgaggtgggagaatcacttgagcgattgcttgaggtcaaggctgcagtgagccatggccatgctactgcattccagctggggcaacagagtgagaccctttctcaaaaaaaatcccccccaaaaaaaaacccaaaaacaaacaaaaaaggtctgctgttgtgaagttcaacccaatccagccccttcccaagttgtcacaaattccaacgtagttaacagtataccaatgagtgataccacaggaaaaatattaaactgatctgagggatatggggcttggaatctaagaaaattggaagggaaattgaaaaggaaattattatttctccttggggagatagtttctaaaattcttactacaccctggggtcagagctgttgattttaaggatagagacaactgagtcacaggaaactattcatatataaaagtacctggcatccaaaaccacacttgtataatatgaatctttcaccatctgagtagggcaaatcagtctatctctgttgatcatctgacaaggatagcacactgagaaatagatctgtcttccctacaggcatagctagttgtacaaactaacaagagacttttgtatacacattccatgatgataaatgccaatcactaaagggacgaggagggattggagagttcaccatacagcaaaatagtccagacaggtgaaaggtctatcaaatgccaggctggtaatcaaaactgtagccttttctctaaacaaagtttagaaccatgattgtgtgggacattattttaataagggaaagtgcagttaatcatgaccccacctttagtccaagaacaaaaatcagagctgccacgtattaagtacccactctgtgccaggtgcagtaactatgcaaaagatgggttttccagatgcaagaaccttggttcagaggaccctgctcaaggcctcatagctaacaaatgatggggcaagatgctatcccaaatctctctgacaacaaaactcattcttatcactctactattttcatagagttgccaaatgcttggttatgcaaacgatgcaggcaggggcaagacagcggctgagcttggaactttttcagagatgtttcctttgcttttagttcacagaaggctccttattggatgggctgtactggaagatgcagcccagcaaccagactgaagctgacaaccgaagtttcatcttctatgagaacctgctgttaggggttccacgaatacggcaactccgagtcagaaatggatcctgctctatcccccaggacttgagagatgaaattaaagagtgctatgatgtctactctgtcagtagtgaagatagggctccctttgggccccgaaatggaaccgcgtaagtgtctgtgactcattgccactcggtgatattcattcatttattctctgaactcccaccattcattcattcattccctgacaccttcaccaaggcaaaaataagttcagtgactcttcagtgcttatatttaaaccttggccaacttgacctttgacttcttaagttttcactacttcttagccttcttttagtttctacatgcatatttttcagaagactaaatcgttgaccatataacccctcaaaaattaattatctgagcgtttgaaaatttcatttaagatgccctgggccctgtttttacaggtgcagtaacatcatccactaagttatttaacacaagttttctggttcaggaactctttttataggtcttgcaaacaggtttttgttcagaatggagttatttaatgtgtaagcttgtgaggcaattttttgttaggtttaaagcccattttgttcaaatgtttgagattttaggtatatatttgtacacgtgcatatttacaggggctttttgtacactttggtactcctacttcaaacatcttgtgtattaagggaggtcacttactattttagaagtattgtagttattataaagaaacaagaagacctccaaggccgtttcagggtgggcctttgcggttgctgtccctgggtacgtcactggtcggagtcatcttctaagctttgctcagctaattctgtcggttcatctaggttcttttcttggaaactgagttgcccagaatccacatttgttactatacaatgggcaatcaccttttcaattagtatattcttcttgtaccttccagtatacactctatttaataccagaacccataagaaacaaatttagtaaaaatccaggttgggcacagtttctcatgcctgtaatcccagcactttggaaagccaaggcggacagatcacttgaggtcaggagttcaagaccagcctagccaacatggtgaaaccctgactctactaaaaatacaaaaattagctgggtgtggtgacatgtgcctatagtcacagctattcgggaggctgaggcaggagaattgcttgagcccgagtggtgggggttgcagtgagctgaggtctcatcattgcactccagcctgggcaacagagcaagactccccctcaaaaaaaaaaaaaaacaaatttagtgaaaatccagagctttagaacaaaggaactaaatagtctcaaaggacattatcatccaagttatgatagtgatttcgctttctttaaaaaaaaaattattacagatagagtttcttgatgttgcccaggctggcctcaaactcctgggctcaagcagtcctccagcctcagcctcccaagtagctgggactatgagaatatgccaccatgcccagctttattttgctttctaatgtgcctttttgtagttcctgcaaagcataagcatgccttcatctgtggtaccctttccaatattttatttatctcacatcactaataagataaatttatacagccactgctctgtgccagacattatttaagaagttatttcacgcattatctcatctgccttcacaaaacaactctaaaataggtatcacctccattttatagatgaaaaaactgaggctcacttgcccaaagtgtcacagctaacaaattggactgaaccaagatttaagcagcctgactccaaaacccatgtttcgcctactaaacctcttccatattaattcctcctccatattaattgcgtcgttagggtggcttgtcgacgctctcagctccccatcagtactcaagcttcctgagggcagggattctattttgttaactgctgtattctcaaagccttgaacaatgcctcatatgtaaagatactaata

SEQ ID NO: 232 - DNA, 134 bp, Homo sapiens
  source 1..134: mol_type = genomic DNA
tgacctacggcatgatgagctccaatgtgtactactacacccggatgatgtcacagctcttcctagacacccccgtgtccaaaacggagaaaactaactttaaaactctgtcttccatggaagacttctggaag

SEQ ID NO: 233 - DNA, 1897 bp, Homo sapiens
  source 1..1897: mol_type = genomic DNA
gtatttgcaaataactttgaaagtacctctctatcacagaaaattgttcatttggcttcatcatttcaatgcatgagtatcgacaggacctgctttgcatttaacactgtgtgagacgtaagttatggtgagttgttagaagttactgttcctactctcaaagggggtaaactaacattgagaactttgcctgtgccttgcactgtgctgagtgtttcatatcttaccttatttaatttctatagtctaactctataaggtaagtactaagactatgccctagtttgttaatgaggaaaatgagattcaggatgtttaaatgcgtatggtcacatggctagggaacaagaaaaattgatttttttctagcctgacagctacttcatcctagtttgtaattcattccatgagtcaagattcaataaatatttattgagaatctcctagaatgtaaggccaatgaagggcagtgtggttcttctgtcttgcttcgccttttgtgttttgtctctttgttgatgatggcatgtatccccagctcttagaacagtgcttgattcaaagtaagcacattctttcaaaggtctgctgttggtggggcttggtggctcacgcctgtaatcccagcactttgggaggccaaggcaggaggattgctttagcccaggattttgaaaccagctgggcacaacatagtatgactttgtctctccaaaaaagttaaagaattagcagggtgtggtggtacacacctgcagtcccagctactcaggaggctgaggtgggagaatcacttgagcgattgcttgaggtcaaggctgcagtgagccatggccatgctactgcattccagctggggcaacagagtgagaccctttctcaaaaaaaatcccccccaaaaaaaaacccaaaaacaaacaaaaaaggtctgctgttgtgaagttcaacccaatccagccccttcccaagttgtcacaaattccaacgtagttaacagtataccaatgagtgataccacaggaaaaatattaaactgatctgagggatatggggcttggaatctaagaaaattggaagggaaattgaaaaggaaattattatttctccttggggagatagtttctaaaattcttactacaccctggggtcagagctgttgattttaaggatagagacaactgagtcacaggaaactattcatatataaaagtacctggcatccaaaaccacacttgtataatatgaatctttcaccatctgagtagggcaaatcagtctatctctgttgatcatctgacaaggatagcacactgagaaatagatctgtcttccctacaggcatagctagttgtacaaactaacaagagacttttgtatacacattccatgatgataaatgccaatcactaaagggacgaggagggattggagagttcaccatacagcaaaatagtccagacaggtgaaaggtctatcaaatgccaggctggtaatcaaaactgtagccttttctctaaacaaagtttagaaccatgattgtgtgggacattattttaataagggaaagtgcagttaatcatgaccccacctttagtccaagaacaaaaatcagagctgccacgtattaagtacccactctgtgccaggtgcagtaactatgcaaaagatgggttttccagatgcaagaaccttggttcagaggaccctgctcaaggcctcatagctaacaaatgatggggcaagatgctatcccaaatctctctgacaacaaaactcattcttatcactctactattttcatagagttgccaaatgcttggttatgcaaacgatgcaggcaggggcaagacagcggctgagcttggaactttttcagagatgtttcctttgcttttag

SEQ ID NO: 234 - DNA, 261 bp, Homo sapiens
  source 1..261: mol_type = genomic DNA
tgacctacggcatgatgagctccaatgtgtactactacacccggatgatgtcacagctcttcctagacacccccgtgtccaaaacggagaaaactaactttaaaactctgtcttccatggaagacttctggaaggtatttgcaaataactttgaaagtacctctctatcacagaaaattgttcatttggcttcatcatttcaatgcatgagtatcgacaggacctgctttgcatttaacactgtgtgagacgtaagttatg

SEQ ID NO: 235 - DNA, 1770 bp, Homo sapiens
  source 1..1770: mol_type = genomic DNA
gtgagttgttagaagttactgttcctactctcaaagggggtaaactaacattgagaactttgcctgtgccttgcactgtgctgagtgtttcatatcttaccttatttaatttctatagtctaactctataaggtaagtactaagactatgccctagtttgttaatgaggaaaatgagattcaggatgtttaaatgcgtatggtcacatggctagggaacaagaaaaattgatttttttctagcctgacagctacttcatcctagtttgtaattcattccatgagtcaagattcaataaatatttattgagaatctcctagaatgtaaggccaatgaagggcagtgtggttcttctgtcttgcttcgccttttgtgttttgtctctttgttgatgatggcatgtatccccagctcttagaacagtgcttgattcaaagtaagcacattctttcaaaggtctgctgttggtggggcttggtggctcacgcctgtaatcccagcactttgggaggccaaggcaggaggattgctttagcccaggattttgaaaccagctgggcacaacatagtatgactttgtctctccaaaaaagttaaagaattagcagggtgtggtggtacacacctgcagtcccagctactcaggaggctgaggtgggagaatcacttgagcgattgcttgaggtcaaggctgcagtgagccatggccatgctactgcattccagctggggcaacagagtgagaccctttctcaaaaaaaatcccccccaaaaaaaaacccaaaaacaaacaaaaaaggtctgctgttgtgaagttcaacccaatccagccccttcccaagttgtcacaaattccaacgtagttaacagtataccaatgagtgataccacaggaaaaatattaaactgatctgagggatatggggcttggaatctaagaaaattggaagggaaattgaaaaggaaattattatttctccttggggagatagtttctaaaattcttactacaccctggggtcagagctgttgattttaaggatagagacaactgagtcacaggaaactattcatatataaaagtacctggcatccaaaaccacacttgtataatatgaatctttcaccatctgagtagggcaaatcagtctatctctgttgatcatctgacaaggatagcacactgagaaatagatctgtcttccctacaggcatagctagttgtacaaactaacaagagacttttgtatacacattccatgatgataaatgccaatcactaaagggacgaggagggattggagagttcaccatacagcaaaatagtccagacaggtgaaaggtctatcaaatgccaggctggtaatcaaaactgtagccttttctctaaacaaagtttagaaccatgattgtgtgggacattattttaataagggaaagtgcagttaatcatgaccccacctttagtccaagaacaaaaatcagagctgccacgtattaagtacccactctgtgccaggtgcagtaactatgcaaaagatgggttttccagatgcaagaaccttggttcagaggaccctgctcaaggcctcatagctaacaaatgatggggcaagatgctatcccaaatctctctgacaacaaaactcattcttatcactctactattttcatagagttgccaaatgcttggttatgcaaacgatgcaggcaggggcaagacagcggctgagcttggaactttttcagagatgtttcctttgcttttag

SEQ ID NO: 236 - DNA, 56 bp, Homo sapiens
  source 1..56: mol_type = genomic DNA
gctaccatactctaaagatagagccacagatcccagtcagccatgtgattatgagg

SEQ ID NO: 237 - DNA, 16648 bp, Homo sapiens
  source 1..16648: mol_type = genomic DNA
gtaagtagaatatttccttgcactaatgggaaagttttgaaaagatttgacctatccaaatcataattaaaaggaagtgtgtatgcaccagaggggcaactgggaagttaccttcttacctttgtttttaattctaatatttttatttgggcatttgtttattgactatcttcctatggtagaatgcaagctttataagagaagggacgtgatttgttctctgctgtacccccatttcccaaaactgcagatggcaacagaaggctctgaaaaatatataagaaagaatttttctaattgtgactaaattgtgaccaaatgctaagtgactgtggacttgcgtttaacacaggacgggagaggcaaagagttcaattccaatttagaatttggtcaagttctcttctgcactctggtaaacattaattaaaaatcagcattatctgaccagccagttcatcgtcagtggtggtgattttcactatgagatacgcgtggcaacttgccagacaccaagaaaccaagttagaggatttttgtattagattccttaacaatgaatacagtatcaccattattacagtatcatcattattgtcatactattattatatcagttaacataaagtctgcataagaattgtttccagaaaaatgactttccaaatttaactttcaggaaatacaaataatgctactaatattgcttttattggcgtatacatgtaatatccccttcttttggatttggatatgttgtgtcattgcctcattttaattcattatttcttctcaatctttaataattgctggacttttactccacaagaaacttgctataggcccatctctttcgtcttctttccttctttcagttcgtcttcccatcctctggtagggggaggggagggatgcctgagcgagagactagctgtaggaaccatttgtctcaaagtccagaaagccacaggtgatggatttgtcctctgaatcaaagggcgttcgatgatggatttctgtcatgtctcatctaaagtcttcacgagaacagatgaggaagcagttttatgaccccagagcctcctaccaaactcctctgagaaaaggtttccttttttttttttttttttaaattagagacagagtcttgatctgtttcacaggccggagtgcagtggcacaatcatagctcactgcagcctcgagctcctaggcttcagtgatcctcccacctcagtcacccacgtagctgggactacagctgcacaccaccatgcccagctaacttttaaaacatttttgtagaggtggggtctcactttgctgcctatactggtctccagctcctagcttcaagtgatcctcctgccttggaagttctgggattataggcatgagccactgcacccagcctggatgtgatatttttatgttttaaattgttagagtttagaaacttgagattgagtttgctgcctgcattaaaatgatgcttaaacattaaactgcagtggccttaaatattaacaagttgattagaattactaagttcttttcaagctttacatatacagacaaatttcttatgcaaaatagaaggtaacccctgtacgtaagtctagaatttcagcagtccccaaaactgactgagcattagaatcactctgattttaaaatacatatgtggttttccgagatctactaacagagcctccataatgtagcctagagaaacgagttttcagatgtagtgataaatttggaaggtaagtcagagaagtaagctgaagacagagttttaggaaatatgcctaaagtcacataatgaattggttttcttgtttatttgagaatattgtcgctttttgttctttttcatgcaaatcacattttatttcttatgtgagtagctatatatttaaaaattttgtttttggaatattgtagaatctctacttaagaaagtatcttagcagtcatatggtctgacctcactgaatgctaaattctctttaaaacatccgtctcagatggttactcatactgcctctgattgaacaagagtcccaggttaagggacttacttctttgaaatcgttcatttcatttttctacagctatgttagaaagttcgtccttagcagtgaagccagagtctatctcttataacttctacccagttgcaccctccaagcctacctataccaagtatcttttttccacgttgcttttccaattcagctcttcgcaagtttggagacaaatacctaatctcctctaagccttctccaggttaagcctttccagttcatccagctgttgattatgtgattggagacacaagtttgagtaactcccatgatggaaagtccctctggtgaatgttctgttcatcagagtccctaagaaagcacatgagcctcaccatggtgagtggggccatgagattcctaaaccagacactacactgtggcttgtgcaacctaccatggccagactcatgaggctgtttcatcatataaataattccttagtctcttcaccaaaaactgtgaagcactgtgtcccccagctgtatgtgagctagcctgggaccatagtggaggactcctctttcaaccctgttaaatttcatcttgttaggatctgcccatttttccattctgttgaaattatcttggaactggattcattcatctcacatcagctactccccgaagcctcacagtatcagcagagttattatctctatccatatccttgtaaattattaaactaaaaaagattggtctaagaacatcagtccattaatcaaggctctttggttggagttcacttcattatattatcatccagctcagagtgttcataaacaagatgataggaaagacttttccaaatgcctgtctgagtaattcccccattcctgtgatctgtgagctgttgaccagattaaaaaggaagtgagaataaccaggcaagatttactcctagcaaaaccttactggcttctagtgactgagtcctttccctattgcttaccagctatctttttaattactagttttaaaatcttgccagcaatgtcaatatcaaatgaccaagaatatcaaactcattactctgtatgtaaatgagatagtgtacttacccctatggcttaagtattaggctgttcttgcattgccctaaatacctgagactgggtaatttataaaaaaagaggtttggccaggcactgtggatcaggcctgtaatctcagcactttgtcaggctgaagcaggtgtaatggtgagccaagagttcaaacttagcctggacaacaaggtgaaaccccctctctgcaaaaaatacaaaaattatctgggcatggtggcatgcacctgtagtcccagccacccaggaggctgaggtgggaaaattgtttgaagctgggagtcagtgattgcagtgagccatgattgcaacactgcactccatccagcctgggcgacagagcaagaccctgtctcaaaaaataaataaatgaataaataaataaaataaataaataaatagaaaagaaaaagaaagaaaagagatttatttgcctcatggttctgcaggctgtaagggaagcatagctccagcatctgcttctggggaggcctcaggaagctgttactcatggcagaaggtgaagcaggagcttgcacatcatgtggcaaaagcaggagcaagagagagagaatggggcagggaagaagccccacacttttaaatgaccagatcgcatgagaaatcactcgttacctcaaggacagtaccaagaggatggtactaaattcctgagaaatccacccccatgatctgatcacctcgtaccaggccccgccttcagcattggggattatgtttcaacatgagatttggatggggacaacatccaaactatatcaccttgcatagtaggtagggttttaaaaagcagtttggcacagtaagaaaagtacagattttttttgcatcagacagacctgagttaaaatcccagcttcactgctaacatgctaggtaaatgtgggcaagttaattaacatttctaagcctttgtttcctcactggtaaaacaagtatttggaaatatcattgtgaagattagaaataatacatgaaaagatcctaggatgctgtctgtcatacagtagtagtagtaagaagttattcttgccaaagattgttgagaatggcagaattatctcagttctaagagctatagtttctaattatttgagcctagactcagattcatttggagcagctaactgctcaccaagagcttattttccatcttaccaatgaggttatgtgccctgtgtttttaaaatcagtctacttaaccaagagaacagaaatgacatgagaattaagtaatctcactttctctgttatttaggatttattcctactcaaaacctgagagttgctatgaattcaccattaaagcacttattaatatacatgggttactgttataaatagcaatagtattgctattgtgtgagttaggtgttgaagttcaagaaaggaataaagaatatttagaagatctttgaaaacagtgtctgggtacggtggctcatgcctgtaatctcagcactttgggaggccgaggcaggcagatcacttgaggtcacgagttcaagaccagcctgggcaacttggcgagacctcgtctctacaagatatacaaaaattagccgggtatgttggcatgcacctgtaatcccagctacttaggaggctgaagcacaagaatcacttgaacctgggaagcagaggttgcagtgagccaagattgtaccactgcactccagcctgggcaatagagcaacactctgtctcgaaaaaaaaaaaaaaaaaaaaaaaaagaaagaaagaaggaaggaaggaaagaaaaaaaaaggaaaaaatgcaaggaaggtatttggtgaatctataataataaaaatgtatttgtcatttcctttttctgtgctctcattctataaaattgagtaaaaaatctatatatagtttaaacacattaatagaaatcacaaaagttagctgagtcaacattgtagaaacataatatttctgtatgtcaaagaaatagacaacattaaaaagcagaaagcaattacaaagaatgattataagaaacatcacaaagggttaatattttaacacatttgaaactcaaaaatcactgagaaaagcagtagacttccataaatattttatagagtagaaaaaataggccaagcacagtggctcatgcctgtaattccagcactttgggaggccgaggagggtggatcacgaggtcaggagttcaagaccagcccggccaagatggtgaaaccccatctctactaaaaatacaaaaactagccaggcgtggtggcaggtgcctgtaatcccagctacttgggaggctgaggcagggaattgcttaaacccttaaacccgggaggtggaggttgcagtgagccaagttcgcaccactgcattccagcctgggcgacagaacgagactctgtctcagaaaaagaaaagaaaagaatagaaaaagaatccatgggcaggcacagtggctcatgcttataatcccagtactctaggaagccaaggtgagaggatcaattgaggccaggagttcaaggccagcctgggcaacatagcaagactttgtctctattaaaaattttaaaattagccaggcatggtgacgcacacctgtagtcccaattacttgggagcctgaggcaggagaactgcttgaggctgcagtgagctatgattagaccactgcactccagcctgagctacacagtgagaccttgtgtcaaaaaagtaaaaaaataaaaattagccaggcatggtggcacatgcctgtagtcccagctactcaggaggctgaggcaagaggatgacttgagtctggaagatggagactgcagtgagctgtggtcatgccactgcactccagcctgggtgacagagcaagaccctgtctcaaaaaaaaaaaaaagaaaagaaaagaaaaataaataaaatttattcaaatacaaaagtgatgtggtttgactctgtgttgccacccagatctcatctccaattgtaatccccgtgtattgacggaggttcctggtaggagatgattggatcatggggatggtttcccctctgctgttctcatgatagtgagtgagttctcatgaaatctggttgtttggtaggtgtctgtcacttaccccttctttttctctctcctgctgccttgtgaagaaggtacttccttctcctttgccttccaccatgattataagtttcctgaggccttcccagccatttggaactgtaagtcaattaaacctctttcctttataaattaccgagtctcaggcagttttttatagaagtgtgaaaatggtctaatacagagacttggtaccaggagtggggtactgctataaaaaataacctgaagatatggaagcgactctggaactgggtaacaggcagcaattggaacagtttggagggctcagaagaagacaggaagatgtgggaaagtttggaatttcctagagacttgttgaatggctttgaccaatacactgatagtgatatggacaatgaagtccaggctgagatggtctcaggtggagatgaggaacttattgggaactggagtaaacgtcactcttacatgttttagcgaagagactggcagcatttttcccctgccctagagatctgtggaactttgaacttgagagacatgatttagagtatctggcagaagatatttctaagcaccaaagcattcgagaggtgacctggcttttcctgaaagcatacagttatatgtgctcacaaagagatggtttgaaattggaacttatgtttaaaggggaagcagagtgcaacaaaagtttagggagtttgcagcctgaccatgtggtagaaaagaaaaacccattttctggggagaaattcaagctggctggagaaatttgcataagtaacgaggagctgaatgtgagttgccaagacaatggggtaaatgtctccagggcgtttcagaaaatcttcagggcagaccctcacaacacaagcctggaggcctagaagggaaaaatggtgtgagccaggcccaggcccaggccccagctgttctgtgcagccttgggacatggcaccctgtgttccagccactccagctccagctgtggttaaaaggagccaaggtacagctggaccattgcttcagagggtacaaatcccaagcattagcagcttccatgtggtgttgggtctttgggtgcacagaagacaaaagttgagctttggaagccgctgcctagatttcagaggatgtatggaaacacctcgatgtccaggcagaagtctgctgcaggggcagagccttatggagaacctctgctagggcaatgcaggggggaaatgtggggttggagctcccacacagagtccccactggggcactgcctcatggagctgtgagaaaaggaccaccatcctccagactccagaatggtagatccaccaacagattgcactctgcgcttagaaaagctgcaggcactcaatgccagcctgtgaaagcagctgcaggggctgtaccctgcagagccacagaggtggagctgtccaaggccatgggagcccaccccttgcattagcatggagacaggggatcaaaggagattttggagatctaagatttaatgaatgccctgtcgagtttcagacttgaatggggcctgtgacccctttgttttggccaatttctcctatttggaatgggaacatatacccaatgcctgtacccccattgtatcttggaagtaactaacttgcttttgattttacagactcaggcagaagggacttgccttgtctcagatgagactttggacttgaacttttgagttaatgttggaacgaattaagacattggggttctgttgggaaggcgtatttggttttgaaatgtgagaaggacatgagattttggaggggccaggggtagaatgatatggtttgactctgtgtctccacccaaatctcatctccaattgtaatccccatgtgtcaagggagggacctgatgggaggtgactgaatcataggggcagtttcccccatgctgtttgcatgatagtgagggagttctcatgagatctggttttttggtaagtgtctgggcttcccccttttccctctctctcctactgccttgtgaagaaggtacttgcttctcctttgccttctgccatgattgtaagtttcctgaggtctccccagccattcagaactgtgagtcaattaaacctcttcctgcctattctcaggcagttctttatagcagtatgaaaatggactactacagaaagtgtgtaactttaaactcagtagtatccaaagaagtaatgaaaatggagaaacgaacaacaaaatcatagtacaatatggtgtatgtactaggacaggaagagcccttttaagaagagatctatgtatttccatttgtttatctctgaaagaaagcaactttgccttgtattctgaaaaagaaaggaatattttattttacttgtaaaaatcttacaaggatgctagtctaaatatagttttcctaatttgccagagaatccatgaagatcgagttgataacaagatcagtgaagtaaaggtcagtgagttaatctcacagcagctgcaggctaattccatttccagtgaaaaacgtcttgattgctcaccacatatcttttcaccacaaacagtttcagtcttaagatcacatgttgcaatccatgagaagtaactattaagccttcaactatgactggagggctcctcgccctttctgataaattgactggacaaaaactcaattttaaaatgacaagaaatagaagatgtataaatgtactttaaatgtgaccaaaatgggttgtgaaaacacaagacacaatatccaaaaatgctggcaacacagtacactgtagagtattggttgtttatttacccttgctattgtgtggctgagcttactgccactgcccagcattgcaagggcatcaaactgcctatcactagcctaggaaaagatcaaaattcaaaattctaagtacagtttctactgaatgcttatcacttttgcaccattttaaagtaaaaaaatcagtaagttgaaccatcatatatccaagattgtctgtatataaatattatacatctttctctcacttttaaaacaaaataatactagccaatactaccattctcaaaagcacttgtgtcaacagcctttaccccttaaagattttcctcacaattttaaaattgttacttactattttctttgaaatgttgaccaaacctggattaaaagatttgggggttttagtgactgtatttcacaaactctcttattgattctgcagcctcacttctgcctcctaaaaagccctcaccaaggtcacgggggatggctcttttcagcctcttcctggcatttggtccagtggcatttggcattctaggacttccctctttgtctttgataactccctctcttcctgtgttcctccttgctgtgttcacttgcttcgctttcttctttctgaagcatgtttacacagtgttttctctgattgggcctgtgacgttctttaggtcatcttttccacaaataatgcttcaactagtacttgcgtgccagtgactccacgtcccactcatgagctctgaacctagtaccagcttctgctggacatctacaatgggatctctcacaggcctctctcattggtaacatgccccagcctgaactcatctcccacccatctatccagccatgctctctagttcacctgaacacttgggtgtcatcctagatgctttcccttcccagtcttctgtgatcattctgcctcatcagaggctctctaatctgtcttctttcctatatcgctcttgtccctattttaatcctaatcatctatttcctgacttattcattccttaagttggtcagtaatttaattaaaaacagatttaggccctgaccttaaatgtgataagtgatatgaaaggagatgactggggaaaaggatttccctcaaggaaggcctctgtgaagcctgaagcaagaatgaaaacgagtcagacgaagagagaattgtatgaatgaaggctctgaggcaggaaaacactcagatcattccagaatcacttagaagccaagtgaagccagttcctggagagcagatcatcaaatgaagatggaaaggtgaccaggggccagacctgtagttttggtgggccttggtgagggatttacagtaggacaccccatggtttaagtatgaaagtgacaagattcctttaagttttaagaggcctcgaaatatgaaccacagattagatggaagctactctccctgtgtctggactttttagaatttccaagagctgctgtttctggaaccagattaatacaagtcagtcttccatttatttatttatgtatttatttgagacagggtctcactctgtcacccaggctggggtgcagtggcatgaacacagctcactgcagcttgggggctcaagagatcctcctgcctcagcctcccatgtagttgggaccacaggcacctaccacccagctaattttatttgttgtagaaatgaggtctcattttgctgcccaggctgttcttgaactcttgggctcaagcgatcctcctgcaacatcttcccaaagtgctgggatcactcttccatttaacatgctatctcaacgtcaagataaactttaaaatctttagataataggctggcattttacttaaacgatctttacttcttcagaactgccattccctataaatatctggttcttcaaccacatcaaaccacttgtgatctcaaaaagcctcagcgtacactgtcctttctgtcattctaattcctcctcatccttcaaaatcaactcaaggaccagatccagggagaagcttagtggtgcccacccgaaccggccccctccttcgagttgtgctgccattcgggcccacctcttcacacagggttgtcagaccagaccagctcatgcgttcaccgcccttgcagggatgggatgcagctgtgcacctctcagtgccgacacctggagagtctcacgaaatgttgacaacatggcctgtttccatttcttgttcactaggacttctcatttactaacacacagaatttcctgtagtatgtccacttaatcagttcaagcctaataattccttgatttgggtatagtgctttgcatttatatactgatggtccccaacttacgatggttcgatttatgatttttcaacttcatggtgatgtgaaagtgatacacattctatagaaaccacactttcaattttgaattttggtctttttccaggctaccatactctaaagatagagccacagatcccagtcagccatgtgattatgagggtaagcgaccaatactctacagtgtattgtattgccagatggttttgcccaactagcctaatgtaagtattctaaacatgtttaaggtaggccaggctaagctgtgttgctcattcagtaggttaggtatattaaatgcattttcaacttatgatattttcaatttacaatgagtttatcaggatgtaactctactataagtcaaggatcatcttgtatagcacttttaatttatagagtcccttcaaatgtttgtttgtattttatttccactgcatccctgtgaggataccataagttatacagctaacaaaacagttagttttcctgtgcaaagtgatggcttcatcttgtggcagattacctggaatactgtggccaaggcatcttagttctactgtctttatatatctagtacagttatatttttatggcagctctgatttcttctttggcccaagggttattaagagagggaaaaaatttaatttcttaacagatatatatatctatgtcaagtcatatatttaattcaaacccttaatattcctaggtaatttttgtctactttctctgtcaaagattgaaagatacagggttttaagtttccaactgtaattgtagttttggtaattgttttattactaacaggtattgctctgtatattttgatgttctgttattcaatacataagaattcattacagttgaatcatcatgtatagtataatttaccaatgtaaaataacctttttatcaaactgagtattcaccttataatgtcctctgtcagaggattataataccacttaaaaccttttaaaaaatattttgttcctgataattttagctttgagtggttttcttataacaattctagagatgcgtttttattttttaaccaagatttaaagctttgtgatttagtaagagtctaaaccattcacagttcatgcttttttgtcaaatttctattatttagtattttcctctcttttatattttcctgttattttccatttctattcttttgttagactggaaattttttctttgctttcttttatcctagtgatttgaaatttatgtaatatatactattctacaataccctttatttgttttcaatatttgaacctatatttttcaacattattaagaataaaatagtatttgttgctattttgaaatgatagaccatgtttttaaggcaggttggtggttgttaaggcaccagtatcggccaggcacgatggctcacacctgtaatctcagcactttcggaggccgaggtgggcagatcgtttgagcccagcactttggccgatactgtggtttactgtattttgttctagtttattttatggaaaatgggaattcagtggttaagacaaggattaaatagcagaagaaagatgtgtacatatgtacagatgtatgtgtcctttatatgttttttagtactcttgtctcctttctggtcctcatttaaggttatctatttcatgcagtaaatttttcttcacaattcatttcatttagagagtgaatgctaccttccaagtgggctttctccagttttcctttcagggacttaaaggagaagtgatgttaacagttttatatttccattgcattttacagtgtgcagatgtcttcacatatatttccccatttgagctttacaaaagcccttagtattattctcattgtctagattccaaaatcaggcttagaggagttaaatagttgtccaggatctcaagatgcaagacccacaatcatgaacagaggcagatgttcaggatggaggcaagctgaaactcaaaaccaaatcattatgactccaaattcaggagtcttttagctgccacctgcatgggctcttggtgtagctgaccaccagagtttgtagagctgtcattcaggtgtgccatggactttcctgggacctggcacaggagaaggactgagttaatgtttgctgattaaatatctgttacaggctgggcgcggtggctcacgaccgtaatcccagcactttgggaggccgagcagggaggatcacttgagctcacaagtttgagaccagcctgggcagcatggcgaaaccccgtctctacaaaaaatttgaaaattagctggccatggtgatgcatgcctgtagtcccaggtactcaggaagctgaggtgggaggatcacatgagcccatgagattgaagctgcagtgagctgagatggtgccactgcactccagccttggccatagagccagaccttatctcaaaaaaaaaaaaaaaaagttacaataatcttcccttcaaagctggaaggcattatttacctgtctgtccagcagatggtgctacataaccaagggaatctgttgcttgcccttggtgaagctattaaagccaatacagatcttgagaatttcaaaagcaaaaatcaatactggattatgagtgctctaggaaaataaagagataaattttcaatttacatacttatatatagttataccatatttgtaaataaaaatatataaataatttatcaaaattcctttttaacagcaacaaccacagtaaacccacaggttaaaaactccacaacagtctatattaatcagtcaatgcaaagtacattccaattccaagttaactgaaaataatcaacttaatcatttggttggctctgagcagccttcactgcttgctcttgtgtcatgtttctttctgtcctcgattggctatagacttacagacggcttttgcagaggacagtgtactcatgtccatcctttgcatcctttgtgggagttggctaggcagcactctccctggggacactatgaactcctcttcttgagtgcagagatcacgtcttgttcatcttcatgcccaataccttgttccataaatatgaatggattagaattctaaactcttaactctgccccaagacagttctgagaggtagtaagtcatataacacctgaagaggactgttcttgtcctaattacattaggttataagatgacaggtgagggagccaaaccagggggcctggaaattattcatacatctctagatacagtatacaagttgtgtgtattatgtgtatttactctgtaattgattgcttgagatgaacccccaaacacactcgtgtttggatcattattatctacccttctccttaaataatcttaatttcctatgatgcttgaaagggaaagaggggccaggtgtggtggttcacacctgtaatcccagcactttgggaggctgaggtgggagcatcacctgaggtctggagttcaagaccaacctgaccaacatggtgaaaccctgtctctactaaaaataaaaaatcagctgggcatggtagcacatgcctgtaatcccagctacttgggaggctgaagtgggagaatcgcttgaacctgggagggggaggttgcagtgagccgagatcactccattgcactccagactgggcaacaacagtgaaactccgtctcaaaaaaaaaaaaaaaaaaaaggcagagtggggaagagagctgcatgaaggagagatttactaaatagtacttaatcccaaaataatttctataggtttgaatatgatccctgaaatttattataggttcaggtaagtattaatcacgggtattcagaactgtggtttaaaaaatgtatagaacatgtttccttccccttgaaactttttatcagctaattataggaattatattatacctgcaatcattaaagtccagaatgagacagtacttggtaaagtgctgaaatttataataaatgcattatagcaatccagttaaggaggaagagccaccattattgaacatttgttaagtgtcagccattgtactagataaattttagttattatttttatttaggcaccaaaaaatccatgggatagttggttatccccatcttactgaagaggaaactgaagctcagaaagtttaagcaacttgcacaggtcacatagcaagtaaggagcatggccaggaatcagaccctgatctcctttggtctactaagcttgcaaaggatcttcccgcctccttccaagaccattcaatattatcagtaaatgtccatggcaaggatgtagttcgagttatagggttccattcaagatatgattggtaggtgggaagcagatatgtctgtgtcaatcagtatcctggaagaaggagatgatgaactcaagtggtgattaagggaagtttaatgaagggactatttacagagatgtggtggggttaagagaaccaacaaggggaagtgatgcactcaaaaagttactacctccaggctttaggggattgggggagggagtggcacagtgtgaacccagtgtggttgtgagaaaagggattccctcagcagccatggccaaggttagagtctccactgccaaactgcatccaggtggtgagggaatggagaataggggtggggtaacaaactctgacctcggtatccccaaagggcaaaggattccaggtggtacagttcgtaaagattagcctcagggcacagaacagggcagagaagaatggagaattgatctggaggaaacaaacaatggcttgcccatgttattgcagggagagtaggctggtgtgcacagcaggagggtggggagcccagcatatagctgtgttggggcctgtgcagatcagcctcactggcagggaggatctgagccgagaggtggtggaagatgaaatcgagtaggcatgttggtagtcctaaatatcaagtaaacgttcctgatcttacattgatactcaatagtaagccaattttgtttcccataagccaatattaatattacgtatttcttttataagccagagatatagagagataccctagaagaatgataggggaaaggaaggcaagggtgagagaagaccttgtgtgaatttgtccaaaatgtttatccacaggaacaatccctttgtgaaggctgctggtatgtgaatgtgtgccggttcccttggggcgttcatttggatctttctgtgttccag

SEQ ID NO: 238 - DNA, 19640 bp, Homo sapiens
  source 1..19640: mol_type = genomic DNA
atgtcttctctctcttacagctcttcaaattcatcaattttaacaggaccatgagccagctctcgacaaccatgtctcgatgtgccaaagacctgtttggctttgctattatgttcttcattattttcctagcgtatgctcagttggcataccttgtctttggcactcaggtcgatgacttcagtactttccaagagtgtatgtaagtatatatgaaattaagaagaaaaatttaatcagagttgtcactgcttctcaagaataaatcttcatatgaggttgctatatgaccaccaattatttaaaaccagttattttaagtaagaattaattaccttttcccaaaacattgatctacccatgcaaagaagacaatgcatcctgaaatgctgatgcttaagatagcagcccaaagtagtaaaatacagttaacagacataggaaaccaacactgttctgaagactgagtttttctttgcaccaaatgcagatggtagcttctagaaggctgtttgcctatattcttactcctgttgaatattgttgccatatatttagaacttcaagttattttctaaggaaaaaaacaagatatttctaatattctaaggtaaactcagaccagtacaagaattttcagtttttttttccaaagatcccaaatgtgaaataaaacaacaaaaagcagccagtgtcagatttctatgccatttagaaaggagttagtttaaaaaggaatggaagtaatagggttttgtgcatagatatctcgaattaatattgctgttgataaaagtgattttgctaagacccagcactgacaacacttggccactttgatcccattttaagtacttgtcagaatattggatctttgaactcaaaccattttgggtttttggggtttttttgttttgtttttttttgttttgttttgtttttgaggcacggtcttgctctgttgcccaggctggagtgcagtggtgcaatcatagctcactgcagccttgaactcctaggctcaagcaatcctgctgcctcagcctgctgagtagctgggactacaagtgtatgccaccatgcctggccaatttttaactttttttatgagaagggatctcactgtgtagcccagggtggtattgaactccagggcctcacactgtcctctcacctcagcttccaaaagtactgggattacaggcatgagccaccacaccaggccctgttgttttttttttaaagaaatttttaactttagaccgagggtgactgttgtcaaggtttagggttaagatgttttacctagattatgtgttgaaatgttatagccaattgctttataagttattgaataataattgtattttctttttttttttttttttgagatggagtctcgttccatcgcccaagctagagtgcagcggtgtaatctcagctcactgcaacctctgcctcccgggttcaagcgattctcctgcctcagcctcccgaatagctgagattatgggcgcacgccaccaagcccagctaatttttgtatttttagtagagacggggtttcactatattggccaggctgttctcgaactcctgacctcgtgatccgcccgcctcggcctcccaaagtgctgggattacagacgtgatccaccgtgcccagcttgtgttttctttttaaccaaatggaaataacctctgtagcatgaaagcattttattattattgcagaaggctttaattgctgatacaagtagcaagactttgtaaatgggattgacaattttctgttattcggcagctacctatactgctaaaaggtccaaaaataatgaaatcatctttaagaaatgttgcatcaactagtggacattctttgtttttgtattgtggtgttttgttttatttttatagcttcactcaattccgtatcattttgggcgatatcaactttgcagagattgaggaagctaatcgagttttgggaccaatttatttcactacatttgtgttctttatgttcttcattcttttggtatgtacatttttatttatagtgaggttcaatttaaacttcgtaaatccttgtcttctcttttctctcacactttatgtcctatcaattttaaataaagacccaggaagtagaaaaaagtgtggatgttggaaaacttattttccttttattaattcacagttttgagactcatatcaaatgtcttttctgtggtctttcattgatccatgtatatgtgtctattcaatgcaaaaaaaattagatctcttccatggtctttcatttctctctctatatatgtatctattccatgcaaaaaagaaattagatcaagtacaaatttataaagatacctaaaatagtgctttgcctaaaaagtagaatatgcttacatgctttttaaactcatatgtcagcactttcgtagtcacttgctagcatgacttttctctctttcttcttttctttttaaaaaataagaacggaaaagcaagctagatctaagatgtcgagtaatagttgagtgaatcattgcatgtcaaaattaggatattctgttttaaattatttatatcccattcatctagagactgcctacagagaatattcaaataattaagtttaaaactaaatgtaacaatgaatggaaattgcattaaaattattttcaaaaataatttttttattctcttgatttggtacaaatgaacatttttaatgtttttgccctaagtcaattaagtttttttaaggtgttttgttctttttcttaacatttatatattcaattgtctactgagaaggtgttaagccagcttaatttaggcaatatttttcatctaaacactaacagtcatcttaagaacaattttcttaagaaaataacattttttccatttcagtaaattgtgtaaagatcccttgaggaaggttaagtgatcacattttcagtaattcagtgtaataactctaaagtcagtccaggtattactggttaagtatatggtatttattgattgggtattagatgtactgtattaatttcctgtttaaaaaaaattttttttccggggagacacagcctctggtgtaaaacaaaggtgtgttccctagctgtactttaacaggactgaaaaggtcaggaatatcattcaagttcatatgtatcttgctgtatgcatggtttatggctcatttttaaacttacacctcttaagcttcttcttcctatcatatattaaaacaatggagagaagaataagcctctgttactctaccattgatagtacttcggattctagagtacctgaatctctactaagaaggcaaaaaccaggaattgagagtcctgcacctgacccttcagttgatctcaggccacctagttttctccgtttatcaatctgccaaacaaggatggatagagtcgtggcaactggaaaggctcaaatgtggaattgtttgaatgtggtcctttagtaggcagccatcttaccagatctagagtattcagtcatcttaccagatcagtcaccagaacatgaaaagaagctcttagtttctatctttatactaaaattgtttttttgtacgactgcacaaaaaagaattgctctccttgcacctcccagagatataggtggatagatacatacatacgtacatacatacatacatacatacatacatacatacatagacacatacatagatagaagtctactttcaatacaaacctgtcttttaaggaaatgacaagctgagcatagggttggccacctttctgagccgattgcctggtattagtttattgcccctgtttagcaagaaggcacagtgttaagaagtggctcagctgaaccaggataaccccactcttcccccacatcaacaggaaagacatcctggtgcagatgtccatctgataattcagggaacctcgggagacaggatggagaggagggtgagctagcttcctcttcccacaccttcaagagcctttctcaagcactttctattttttgaaatctctttagaggtcccagactttgatctgtttcaattaaggtattggcaggcattagttaacagccacttggaagcaaaaatagaacattagatccctgagttggaagagagaaggtagaaggtgttacttggactgcaattatctgcacttggaattgagcatttagtcaaaaacttatatgtattctatattctattctcatttctgctacagaattgtaaacaatattcttccttaatacagaaattcatagcccactaaaataagagcgttctcatttgttcatttctcaatcatttaataagtatttactaagccactatatccatatatatatatatatcatatatactgtataatacacactgtagtgttttttgtggattgtgtactatgaggtagtatgttagatactgccagtactggggtaaggaaaacagcctgattaggcccttacgaagattcctcagacttgtggggaaaacagacattatcaaatagaaatacttgcaaaccacagttatgtgttaaaaaggaaaaacaaagtaaaaaaaaagttggtgggggggaacctgatctcctggatacagtgcttcgagaaagtttgttgttggaaatgcaaaccattactactgtggaagggaaaggtcagaaaaatgaactcaccattactgaatagtaatagtagctatcaattaggtggcacttacctgcatcaggacctgtcctgagcactttacatagattgtctcactaaccagcccaacaaatatgtaagggagatactattatttttcccattttattaatgtaaaacaattaaataattctttaaaattagacttagaaaagtggagcaacaatcttagcagtgctaggactgaaatccaagtttgcttgactccaaagtctatctctcttccagaaactttttctttactatctgcctagtaggcctgctgtattcctatttgcaacagccttttaaactctttaaaaatgtgtcctgtaaatttcatatatgattatacaaaaaaacttggaataagcatacaattctacttatctgtgttaactgttgaaatttgaagagctttttggaattctatacccttcagtagtgtatgtaaaagtttctaaatatagagaacatagataagcaaaaataatattaaataaaataatcgcaccattagtaggtaaatatactaatattttgttgtattttattcttgtatgttttcacaaagtatatcataaaatttttcctgtggcatgacttaacggagaaaataatcttcccaaaacatgtggcagcaaaactgttaatttattacatcaggctgggcacagtggctcacgcttgcaatcccagcactttgggaagccgaggcgggcagatcacttgaggccaggagttcgagaccagcctggccaacgtggtgaaacactgtctctactaaaaatacaacagttagccaggtgtagtggcacatgcctgtaatcccagctactcaggaggctgagactcaagaattgcttgaacccaggaggcagaggttgcagtaagctgaggtcgcgcccctgcactccagcctgggcaacacagtgagactctgtctcaaaaaaaaaaatttttttttaaataaataaataataaatttatgtcttcataaagcactcagattaggaaaaaaaggataaacaaaaaggcatgtgtcatttttttgattgataattccaaattatgtttcttcctttaatttttgccctcctttcatttacaaacagaatatgtttttggctatcatcaatgatacttactctgaagtgaaatctgacttggcacagcagaaagctgaaatggaactctcagatcttatcagaaaggtaggaaaaaccttaattctcagaattcttctgtttctgacataaaatgagcattgtttcacccagattttcaaatcaacattgatccattgaaattgtttgaaataaagaatacattgctatatttcaggaataatttaaatgttccctatcttggagtcttgatggatatactgctatcttgaattttaattctgggaatccttttatgccctggaattaaattctcaacaatcttttgacactttaagagctgagctgaaggttcatcaccttcattattttgacatctcctgtagctggctctcacttcaggatcctgagttgagaataaactagaagggaagattatataaagggatttccacctcttctgtctcaattaccattttaaaaaaataaaaagttttagaggaaaacacttagtagttcaccctttacccttgaccttccacggcagttttaaaataagcaaaggaaaagattcatgaattcaggccatagcctggggcctgagaacttttacttatgcaccttctcaggaagggtttcattgttaaatagaagggcaggacaggaaagttgggcctctttgttcttctcaatgtaacttctttatttggtttaaagtataaaatgtatacaacaacaaataaccacatttaaaatacacagtttgtttcccaacatcattttgctaagtcatagtggctccttaactgtaatttttttttttattagtccaagccttaggattatgttatctgtgatatatgttataatagaaaacttaagcctcttaaaacaaagtccttgggatgggacctaagattcacattatcttgattccgcataacagttgcttacattttagcaaatctccagtgtgtatgcaagcactcctcacttggcacaattctgatacacacaaactttggttaccccagttttgttatgtaacaccaccttcaacaacacagttcaaatttcagttatcatagtatattaactctgagtaacagcacaaagtacaaactccactgctagctcttcagtgtatagatcagttacctgagtaacagatgtgcaggctgagcaggctcactggtcagtcatgacactattttcagtctgttactaattggtcactgagcatctgctatccaattcacaaacaaagaaagcatgtagtgttgcttccttgtgttccagtaataagcccatgtgacattttacaaaaatggataattgaaaaagagaatgggtgcagtggctcacgcatgttgggaggccaaggcggatggatcacctgaggtcaggagttcgaaaccagcctggccaacatggtgaaaccccatctctactaaaaatacaaaattagctgagtgtggcgacagatgcctgtaatcccagctactggggaggctgaggcaggagaatcgcttgcacctaggaggcggaggttgcagtgagccgagatcgtgccattgcactccagcctgggtaacaagagcgaaactccgtctcaaaaaaaaaaaagaaaagaaaagaaaagaaaaagagaatgggctagcaaagaaatgaaaaatggtaacactggaagtgaaaatcaaaacagagtaatggatttatagaagaaatagctgagtgaagaagaaataggagtgttgacactgcgatcattcaagagatccagatatggagccagaagaacttagggcaggtctatcaacttaaatgaggaaaatagctgtgataaaacagatgaagatgtcttgaggaaatgatgcctgcaaaaaacttcacattaagggaagtcttattagagatatttcacaataatgaaagtacaaaagaaaaaatgttggggctggacatggtggcttactcctgtaatcccagcactttgagaggccaaggtgggtggatcacttgaggccaggaagtcgagacaagcctgatcaacatgatgaaaccccgtctctattaaaaatacaaaaattagccagacatgatggtgcacacctgtaattccagctactcaagtggctgaggcacgagaattgcttgaaccagggaggcggaggttgcagtaagctgagattgcaccactgcaatccagccaggtgacagggtaagactgtgtctcaaaaataaataaaagaaaaatatgttggaagctcatccacatttaagaaggaatatgacaattcactaatgcatagaaaagaagttcactccacattgtaaagtgtacagtgtaatattatacaatgaaaacaaggcaagtgctgtttaaactactctggatacattttttacaaagaaataaaacactttagtttttaatgtttctaatgttttacattttagtgtattaaatcaatattagttttcttcttttttaagctccctatacatttataactgacactaagggagtgtttaatgttttgattaaaagttgtaaagatcacagaacaattgtaattcttcccactgattattcagatcattttgcacaatttcagcttgcatggtcacttacagtgccgcactatgtgcaaagcaaggtcaggtctaaagttcgctaatgaaaaatcctcggccaggggcagtggctcacccctgtaatcctagcactttggaaggcgaggcaggcagatcgcttgagctcaggagttcaacaccagcctgggcaacatggtgagaccctgtctctacaaaaaaaaaaaaatagcaaggcgtggtgactcacacctgtagtcccagctacttgttgggggctgagttgggaaaatcacttgagctcaggaggtcgaggctgcagtgagccagaatcacgccactgcccttctgcctgggtgacagagtaagatcctgtctcaaaaaaagggaaaatcctcatctacatttcactgggttttttgtttgtttgtttgtttgtttatacacacttaaggaaattactgtctagaagatagataatataaaaaataaaaatgcaattcatgattcgggtttcttggtattcctaagaactgttgcacagtactttatgctctgaggcagacagctatagcatatatagtaatttttgtttctatcacataaacttgaatacacatatgagtaaaagacctttagttcttcatgacttactgaaagaccctgactttttccatgtaactgttccacaagtgttttatggaaaactggatacattaattcttcattcatccagcacgtacttgttgaatggccaatgtacaccaggtttgtagtagttactactgtgaatggaaagtaaaacagatgcaaaaggagaatacactaaaccaagtcttttattttttctctctctgatagggctaccataaagctttggtcaaactaaaactgaaaaaaaataccgtggatgacatttcagagagtctgcggcaaggaggaggcaagttaaactttgacgaacttcgacaagatctcaaagggtgagaatcatgcttcctgaggttctgaaaaattcctgcttctaaagataaattcctggtgataagagtatttctagcccaagggctcatacagatactttttttttttttttccagaggcaggtatctttctggaacatgttataagaggaaaacttgcccccatttggtgatttctcctttcctcctgcattttgatgtctctgtgttgagggtgaactgggtacaaggaatgatttttatctgtatcctctctctaatttcaggaagggccatactgatgcagagattgaggcaatattcacaaagtacgaccaagatggagaccaagaactgaccgaacatgaacatcagcagatgagagacgacttggagaaagagagggtgggtctggtttaggagaaccggatttgatttggtacctacaacaccacagatgtatcaaacactatagaagtagtgggttattgagtctcttgcccattccccaccacactctctctctctctcagtcggtttatgtgttagtaccctgtttattccagaaagaatatataacacaattatgtataaaaatgggtggttagcatgatataaaaacgtcaaaatgaaaagcaagcaaaacaaaagtaaaaataatggattattaatgaagcttaaaaatgcattcataaaaacacatatgcttattaagattgggctacaaattgggccctaagcttgctggtaatcagcttgaaaagagaagcctgattagctgcagagtccacaatgtccgtgagagtgaagaaaacaaaaaatgacttaccaagagatgtgaaattattctggttagttagtggctatttaaattgttaacttttttttcttttttttttttttttgagatggagtcttgctctgcctcccaggctggagtgcagtggcacaatcgcgactcactgcaacctccacctcccgggttcaagcgattctcttgcctcagcctcccaagtagctaggactacaggcacatgccatcatgcccggctaatttttgtatttgtagtagatatggggtttcaccatgttggtctcaaactcctgactgcaagcaatctgcccaccttggcctcccaaagtggtgggattacaggcagtagccaccgtgcctttcctaaattattaacatttataataaaattaacagccgccttccatttgaatactttttacaaaatagttaaaaataaacataagtgggcttttatagtcagaaaaaaaaattcaaagctttaccattaactttcaaaaataaatggttagacagcaacaacaaaaatctgtggtaactgaggtacagagaacacagatgaatgttattacaaaagccactttcctatgagaagtctaggacagtggtttctaaatgccactccacagacagtgctagtaggtgacagacttctccagtcacagtgaaatttaagcataaagaaaatgaggaaaatttttacaaggctctatttagacaaagttcttattctgacattacatctttcctactttggagctgttgaatgtattatcttttatgaaaagaaggcgatccaggttgagcatccctaacccaaatatgtgagtctgaaatgctccaaaacctaaaacttcttgagcacaaacatgatagtcaaaggtcatgcttaaaggaaatgctgtcattggagcagtttggattttgggttttcagattagggatgctgaaccagtaagtataatgcaaacattccaaaatatttttgaaaatcccaaatccaaaacacttctgatcccaagtatttcaaataagggatactcaacctgtaatatatttcttcatttctttatttattttattattattttaagatggctcatggcccactgcagcctcaaactcctaggctcaagtgatcttccgacctcaacctcccaggtagctcaggtagctgggactgcaggcatgcatcaccatgcctggctaattttttaaaaaattttttgtggaggcagagtctcaccttgctgcccaggccagtctcaaactcctggcttcaagcagtactcctgcctcagcctcccaaagtattaggattacaggtgtgaccactatgcctggcccatatttcttcatttagttttttctttgcctgctgtgtttttaatgttctttcttgttcaaacaaaaagttggctattccttgctgttagttaaatttgccaatctatgaaactgaaaaatgcaggagtcccagcctggtgttaaatacaaagaaatcccaggtaaatggcatgcacccagttcctgcttgcccaagtccttggtgaggcttctgtggggtctcagtgttctgctcctcactcagtgaccccttgttcttcaggaggacctggatttggatcacagttctttaccacgtcccatgagcagccgaagtttccctcgaagcctggatgactctgaggaggatgacgatgaagatagcggacatagctccagaaggaggggaagcatttctagtggcgtttcttacgaagagtttcaagtgtaagtataaaggaattggcagaatttgcgttgacaagagtccacatgagaccaggcagttccctcatctctctgaattcactcctttccattactaatcatccagcttttaaaaataacttatactggccagacgcagtggctcatgcctgtaatcccaccactttgggaggccaaagcaggcaggtcatgaggtcagaagttcgagacgagcctggccaacatagtgaaaccccatctctattaaaaatacaaaaaattagctgggcatggtggtgggcacctgtaatcctagctacttgggaggctgaggcaagagaattgcttgaacccgggaggcggaggttacagagagccgagatggctccactgcacaccagcctgggcgacagtgcaagactctgtctcaaaaaaaaaaaaacttgtcaattggtgttttgtttcttacataatatgtttactataaaaattagcaaataagagcaaaagaaaacattaacatttcacatatttctaccaatgaaaaatgtttattaatatatcagtgtttgtgtctctatttgcatgtgtttatcaatgtttctatatatttttatggcctaacatatggttcgtcctgaagaatgttccttgtgcatttgagaagaatgaatattctgctgttcaagtgttctgtagatgtttgttaggtctagtttgtttacagttttattcaggtctcccatttcctggttgatcttagatgtgcctagatgtggtattcacgtttgaaagtggggtattaaagtctccaagtattattagttggagttcatcccttcaattctgtaaggttttgctttgtgtattttggaactctgttgttgggtgcatacatatttataactagtatattttcctaatatattgaccctattttctctctcaacttaatgaggctaaagaaaaaaaagaattgaccctgttttcattacaagatgttatccactttatctctagtaaaattctttgttttaagtattttttgtttgatattactgtaaccactccagctttcttttggttgctgtttgcatgataaatctttttccatccttttactttaaacttatttatatctttcagtctgaagtatgtctctcctgtagacagcatataattggatcttatctttttatccagtttgacaatttctgtttttgattagattgcttaatccattcatttaatgttatcattgatgtagttgatttctgtctgctattttattttttgttttctagcttacttttttttgttcctctttcactgctttcttgtacattaagtgaatattttcaagtataacatttaaatttttttaatgatttttcattttttttagtcaggagttgctctaagacttagtttatacaattaagttatgaaaaattacttcagatatatattaactgaatccagtgagatatagaaatcatttctatttagcttttttcctcttccctctttttgtgctatatattcatctatctatatgtatatatagtcatctacatatgttgcaaatcacattgttagaacaatgttacatttttataacacactgtgtaatatatagtatataattttatatctcttaatgaagctgagagaagaggaagtatatatttataaatgtgtatattaacctacttttttaccatttctaattctcttcttttgctcctctggattcaagttatcatctgtcgtcattctctttctccgatacagctttgctactgcctacctcctattattgtcaaatatattacatttctattatagaccttcagatccaattatgtacatatttttacacaactgctttttaaatcagttaaggaacaaaaggagaaatgtacatatatactatattttatacctacacagttatctttaccagtgttctttgccttttcatgtggattctgattactatctggagtcacttgctttcagcataaagaatttcctttagtattttttgtaaagcaggtttgctagcaatgaattctttcattttttgtttatctgagaatgtttttctttctccttcatttcctctggcttgtattgtttctgatgagaagacaggtgctaattttactgtggtccccttgtacatgatgactcaattttctctcaccactttcaagatttttttgcttttgtctttcattatttttactgtgatatgtctgggtataaatctctgagttcatcctacttagaaagtgttttttctgcttctttcactttctcttctcctttgggacccgcattatgcatatgcttaggggtatcacatatttctcttaggctctgttcgtcgtcattttttttccctctctgttcctcagagtgcatagtctgtattgatgtatcttcaagttcactgacttgttcttctgtcagcttgcttaaatctctgttgagctcctctagttatttatttatttatttatttattttatatatatatatatatatatatatatagagagagagagagagagagagagagaaagagagagagagagagacagggagacagggtctcactccatcacccaggctggagtacagtggtgtaatcatggctctctacagcctgaacacctgggctcaaatgatcctcctgcctcaacctcccaagtagctaggactatgggcacatgctgccatgcctggctaatttttaaaaaaaaatttgtagagatggcatcttgttatgttgcccaggttggtctcaaactcccggcctcaagtgatccttccgcctcggcctcccaaagtgctggggtcacaggtatgagccaccgcacttagcctgaattttttatttattatacttttcaactccagaatttctatttggttctttttagcaatctctgtctctttattctgtatttgatgatatcctgtatttgatgagacaatgtcatcataacttccttttttttttaagagatagggtctctctctgtcacccaggctagagtgcagtggcatgatcctagctcactgcagcctcgaactcctgaactcaagcaatcctcccacctcagcctcctaagtacctgacactacaggcatgagccactgtacccagctaatttttattttttgtagagatggggtctaagttgcccaggctggtctcaaactcctgggctcaagtgatcctccctgctcaggctcccaaagtgctgggattacaggcatgaaccactggacccagcctcctttatttctttaaccatggtggggttttttattatttgtttgtttgttttaacctgtttgaatatatttataataggtaccttcaggtctttgtctgctaagtctgacatctgggccctctcaaagacagtttcagttgtctttttttttttcttcttgtgtatgggaacattttcctgtttttttgttttgttttttgttgttttgttttgttgttgttgttgttgttaaagattggacatgttagataatatattgtaggaactctggctagtgattccctttcctccccaggacttgtttttatttctctttgcttgtttactttttcatacagtctgtttccccacagtgtctgcctctgactttattccttagagggcgcagctgtggccatgtacgtagtcactctgggaagacagtggttttagcagcgtgctcattaactttctctgacctctttgttatacctcctgcctctgtggatattagacccagttattacatttcattgtttactgattggtctattgttttccagaatgccttgggatgtaatttgctccacagtctgatccacttaaattcaggcctctttgcagggctagttttagaggccagtctttttttttttttttttttttttttgagacagagtctcactctgttgcccaggccggaatgtagtggtgcagtctcagctcactgcagcctcaatctctcaggctcaagcaatcttcccacctcagcttctcaagtagctaggactacaggtgcacgccaccatgcctggttaatttttgcattttttatagagatggggttttaccatgttgccccggctggtctcaaactcctgagttcaagcaatccacccacctcagcctcacaaaaggccgggattacagatatgcaccaccacgcctggcccctgaggccagtctttaactgtcttcttagctgtctctttccctggttctctctggtgaactagctggtaatttgtttatctcataaggctaccagattccttgtaaatgcttatccccacaatctccattgttttcaagagcatcgttagtctttaatttcctcacgcttcattccaaataaagtccattcacttgagaagagttctatattcctatgacctgtgtctccccatgagcaaaactgctactgctttacagagccagggacagtggcccacctctctgtggcatcctgctttatgaacaagtcactgggctcagatggcagtctctgattttctcaccttgcttcttctggcatggaaactccaccctacaagtgggaactgagtggaagaagagagccccattccccttagccactcttaacaggattagaacctctgcaacatgcatttaagaatgggaacaggctgggcacagtggctcacgcctgtaatcctagcactttgggaggtcaaggcaggaggattgcttgagccaggagttcaggaccagcctgtgcaacatggtgagaccctcatctctacaagaaatagaaaaattaactggtgggttgtgtatacctgtaatcccagctactcgggagcctgaagtgggaggattgcttgaacctggaggcagaggttgcagtgagtcaagattacaccattgcacgccagcctgggcaacagagtgagactctgtctcaaaaaaaaaaaaaaaaaaagagtaggaacattgaggctgggcatggtggctcatgcctgtaatcctagcactatgggaggccaaggcaggagtatcacttgacgctaggagttcaagaccaacctgggtaacatagcgagactttgtctctattaaaaaaaaaaaaaaaaaaggtgtaaaaaacttttgtaacaagagtgggaaagccgggcacagtggctcacacctgtaatcccagcactttggaaggccaaggcaggcaggcggatcacctgaggtcaggagtttgagaccagcctggccaacgtggtgaaaccccatctctactaaaaaatagaaaaattatctgggcatggtggtgcacacctgtagtcccagctactcgggaggctgaggcaggagaatcacttgaacctacgaggcagaagttgcagtaagccaagatcacgccactgcactccagcctgggcgacagagcaagactctgtctcaaaaaaaaaaaaagagtggaaatgttaggatgagaaatgctggcagcctgcccctccctgggagatactgtagccctagactggaagttgggggaggagggagccctgtgctcttagctgcacccatgtgaagttgtgcttctatcacatgagctggggacaggagagaaggctcagattatggcttcagtgccacagaatctcttcatactaaaatttagtagattttcttgaataaatgctttttcatttgctgtacacccttaaaagtttctagaaatttttaatatttgagttttaaaaaataattttcaacagttacagttatttcactaaagagagagtctacagaaccctcttgccaccattgcagaggttgtctttggcttacgagtttttaaagtatttgtatacattttttaagttcaaaataatagaattgtaagtgaacatgctgttttcatactgtttttcaagctttatttaatatattgtaaatctaattctattttattaaatagtctgccacagtataatgtctgatgtctccttagaattttattgtatggatgaacaatgattatttaatttcctaccaattgttgggtgttttttgtttgtttgtttgtttttgagactgggtctcactctgtcacccaggctggagtgcaggagtgcggtggaatgatcacggctcactgcagcctcaacatcccaaggctcaggtgatccttccacctcagcctcgcaagtagctgggagtacaggcacatgccaccatgcccacctattttttagagatgaagttttgccatcctgcccaggctggctcgaactcctggcctcaagcgatctgcacacttccgcctcccaaaatgccaggattacaggcgtgagccatcatgccctacccccccatcaattgtttgatgtagccatttttcaatgatccgcgattaagaagcagcactcttttatagccaaaaattacacatatataaaattttcctttagaaaatgttctaaaaatggaatgtctaactaaagggttaggcatacattcttaagacttctgatacgcgctgacttgcaggaaagttgtttcagttaacactcctaccagcggcatccgagagttaatctgtaaagcttgagacaacttagaaagtgtttcaaatgattgtgttgcttaagaaaaaaatcttagcacttccttttgaaaagccagtggggctgaaaagacaatgacaagcactttgtccctctgtactgtgttttccttgcagcctggtgagacgagtggaccggatggagcattccatcggcagcatagtgtccaagattgacgccgtgatcgtgaagctagagattatggagcgagccaaactgaagaggagggaggtgctgggaaggctgttggatggggtggccgaggtcagtagtcatgagctgaaaacaccgctgctgagcatggtgttattaatgaaaatatatgttgctgacagttgtatttgaagtattgaagaagagtaaaaaaaatttacgtttatagaaattcacaatgatgtttccatttactctcattttcagatttttttctctgaaacagaaacactctttctataaaatctcttgctataaaacatcaatgtagtcatattgtctaacccttaggctgagatgtttatctttctccataactacagataaaattataatctggaggtgttactttcttaatactccatatgctaatggtcctgccttcactgcagggtagaattaagtgaaaaattactccagcaactctgagatttgctattatatgctgtaaatctccagccttaccaaactacagattatttggtccctggacttcctaaggcatttccttctactgcccccaacaccagtttctttttccctttttaggatgaaaggctgggtcgtgacagtgaaatccatagggaacagatggaacggctagtacgtgaagagttggaacgctgggaatccgatgatgcagcttcccagatcagtcatggtttaggcacgccagtgggactaaatggtcaacctcgccccagaagctcccgcccatcttcctcccaatctacagaaggcatggaaggtgcaggtggaaatgggagttctaatgtccacgtatgatatgtgtgttt

SEQ ID NO: 239 - DNA, 7321 bp, Homo sapiens
  source 1..7321: mol_type = genomic DNA
gaatgataggggaaaggaaggcaagggtgagagaagaccttgtgtgaatttgtccaaaatgtttatccacaggaacaatccctttgtgaaggctgctggtatgtgaatgtgtgccggttcccttggggcgttcatttggatctttctgtgttccagtgacctacggcatgatgagctccaatgtgtactactacacccggatgatgtcacagctcttcctagacacccccgtgtccaaaacggagaaaactaactttaaaactctgtcttccatggaagacttctggaaggtatttgcaaataactttgaaagtacctctctatcacagaaaattgttcatttggcttcatcatttcaatgcatgagtatcgacaggacctgctttgcatttaacactgtgtgagacgtaagttatggtgagttgttagaagttactgttcctactctcaaagggggtaaactaacattgagaactttgcctgtgccttgcactgtgctgagtgtttcatatcttaccttatttaatttctatagtctaactctataaggtaagtactaagactatgccctagtttgttaatgaggaaaatgagattcaggatgtttaaatgcgtatggtcacatggctagggaacaagaaaaattgatttttttctagcctgacagctacttcatcctagtttgtaattcattccatgagtcaagattcaataaatatttattgagaatctcctagaatgtaaggccaatgaagggcagtgtggttcttctgtcttgcttcgccttttgtgttttgtctctttgttgatgatggcatgtatccccagctcttagaacagtgcttgattcaaagtaagcacattctttcaaaggtctgctgttggtggggcttggtggctcacgcctgtaatcccagcactttgggaggccaaggcaggaggattgctttagcccaggattttgaaaccagctgggcacaacatagtatgactttgtctctccaaaaaagttaaagaattagcagggtgtggtggtacacacctgcagtcccagctactcaggaggctgaggtgggagaatcacttgagcgattgcttgaggtcaaggctgcagtgagccatggccatgctactgcattccagctggggcaacagagtgagaccctttctcaaaaaaaatcccccccaaaaaaaaacccaaaaacaaacaaaaaaggtctgctgttgtgaagttcaacccaatccagccccttcccaagttgtcacaaattccaacgtagttaacagtataccaatgagtgataccacaggaaaaatattaaactgatctgagggatatggggcttggaatctaagaaaattggaagggaaattgaaaaggaaattattatttctccttggggagatagtttctaaaattcttactacaccctggggtcagagctgttgattttaaggatagagacaactgagtcacaggaaactattcatatataaaagtacctggcatccaaaaccacacttgtataatatgaatctttcaccatctgagtagggcaaatcagtctatctctgttgatcatctgacaaggatagcacactgagaaatagatctgtcttccctacaggcatagctagttgtacaaactaacaagagacttttgtatacacattccatgatgataaatgccaatcactaaagggacgaggagggattggagagttcaccatacagcaaaatagtccagacaggtgaaaggtctatcaaatgccaggctggtaatcaaaactgtagccttttctctaaacaaagtttagaaccatgattgtgtgggacattattttaataagggaaagtgcagttaatcatgaccccacctttagtccaagaacaaaaatcagagctgccacgtattaagtacccactctgtgccaggtgcagtaactatgcaaaagatgggttttccagatgcaagaaccttggttcagaggaccctgctcaaggcctcatagctaacaaatgatggggcaagatgctatcccaaatctctctgacaacaaaactcattcttatcactctactattttcatagagttgccaaatgcttggttatgcaaacgatgcaggcaggggcaagacagcggctgagcttggaactttttcagagatgtttcctttgcttttagttcacagaaggctccttattggatgggctgtactggaagatgcagcccagcaaccagactgaagctgacaaccgaagtttcatcttctatgagaacctgctgttaggggttccacgaatacggcaactccgagtcagaaatggatcctgctctatcccccaggacttgagagatgaaattaaagagtgctatgatgtctactctgtcagtagtgaagatagggctccctttgggccccgaaatggaaccgcgtaagtgtctgtgactcattgccactcggtgatattcattcatttattctctgaactcccaccattcattcattcattccctgacaccttcaccaaggcaaaaataagttcagtgactcttcagtgcttatatttaaaccttggccaacttgacctttgacttcttaagttttcactacttcttagccttcttttagtttctacatgcatatttttcagaagactaaatcgttgaccatataacccctcaaaaattaattatctgagcgtttgaaaatttcatttaagatgccctgggccctgtttttacaggtgcagtaacatcatccactaagttatttaacacaagttttctggttcaggaactctttttataggtcttgcaaacaggtttttgttcagaatggagttatttaatgtgtaagcttgtgaggcaattttttgttaggtttaaagcccattttgttcaaatgtttgagattttaggtatatatttgtacacgtgcatatttacaggggctttttgtacactttggtactcctacttcaaacatcttgtgtattaagggaggtcacttactattttagaagtattgtagttattataaagaaacaagaagacctccaaggccgtttcagggtgggcctttgcggttgctgtccctgggtacgtcactggtcggagtcatcttctaagctttgctcagctaattctgtcggttcatctaggttcttttcttggaaactgagttgcccagaatccacatttgttactatacaatgggcaatcaccttttcaattagtatattcttcttgtaccttccagtatacactctatttaataccagaacccataagaaacaaatttagtaaaaatccaggttgggcacagtttctcatgcctgtaatcccagcactttggaaagccaaggcggacagatcacttgaggtcaggagttcaagaccagcctagccaacatggtgaaaccctgactctactaaaaatacaaaaattagctgggtgtggtgacatgtgcctatagtcacagctattcgggaggctgaggcaggagaattgcttgagcccgagtggtgggggttgcagtgagctgaggtctcatcattgcactccagcctgggcaacagagcaagactccccctcaaaaaaaaaaaaaaacaaatttagtgaaaatccagagctttagaacaaaggaactaaatagtctcaaaggacattatcatccaagttatgatagtgatttcgctttctttaaaaaaaaaattattacagatagagtttcttgatgttgcccaggctggcctcaaactcctgggctcaagcagtcctccagcctcagcctcccaagtagctgggactatgagaatatgccaccatgcccagctttattttgctttctaatgtgcctttttgtagttcctgcaaagcataagcatgccttcatctgtggtaccctttccaatattttatttatctcacatcactaataagataaatttatacagccactgctctgtgccagacattatttaagaagttatttcacgcattatctcatctgccttcacaaaacaactctaaaataggtatcacctccattttatagatgaaaaaactgaggctcacttgcccaaagtgtcacagctaacaaattggactgaaccaagatttaagcagcctgactccaaaacccatgtttcgcctactaaacctcttccatattaattcctcctccatattaattgcgtcgttagggtggcttgtcgacgctctcagctccccatcagtactcaagcttcctgagggcagggattctattttgttaactgctgtattctcaaagccttgaacaatgcctcatatgtaaagatactaataaatatgtgctggatgcattcgagtgatttctactccagggtggattgccaaaggggaccttccctgtcatatctaaactatccttttattcttctacattctcaagtcactcccatttcttcttcccttcaactccaaacttcttggaagaatagtcttcatttgccacttccatttccttaccatcaattcacgtcttaaagcttaggttcttgcttttgacatctcaccaggaagctactagggacctctagttagcaaatccactgaacaaatctcagtttttatccccttcatactgtctgcagaaagtggcagtgttgaccgctcattcttttgacttccatgaagaactctacactttgggttcttttaactctgaaacccctttctctcacttctcttattcccaggttctatattcatatctccgtcttttctttcttttctctctttcccatgacattcagatggcttccactgacatctttatttggtgactcatcatttcttcaacaatgtgccaggcatgttcccattgctagaagtcccccctcttctagtagtgcccttgatgctggagaaacaaagcagcaggaaacagaagaaacgtcctttcttcacagagcttgcattctagtgatacaagacacatctctattccacctggatgtcccacacctcttgtgatttaatatgccccaaactaaactcatcgttttcttatgagatctgctctttgttctgttaatggcactgccgtcttcctattgccctgaaagagactccagagtcatttttgagccatgtttcctccttgccccaacttccaaatcagttaccaagttctgctggcagtcactgtggttacgacactctctcactccttcctttcccttcccatcttcactgagtgacttcaggcctcattacctcttggttattgcagactttgctaagggagctgtgtcaggggtccctaagaccacccccaggttcagtgattcactagaaggactcagcatagaggcatccttacagctaaggtttattatggtgaaaggatacaaaagaaaaggcacatggcaatatctggggaaaacctggtgtaggcttccaggagccctctcccagtgaggtcacataggatgtgctgaatttctccaggaacgagttgtagcaacatgtgtgaaatgtctatcagagagattaattagagactcagtgcctggggtgttcactgggtactggtactggtttatgactagcatgtgccaaaattcccgactctcagaaggaaagcaggtgttgagtgtaagccacattgcttatacaaatagcacaggcacagcaagcctctcttaccggtcagggaaagttgaatactggtgcagggagctgtttatcagtcaagttcccagatgccagccaaagtccaaccttgcaaacaggccttcctaaagaaaggccatctcaggcctgctgtgttaactcttttctgcacatgagccctctgttcctccccactccagtcttccctgctgccaccagaccagccctgttgtgtgcctcttagaactttcccttcacagactccttcatgtttgcccttcacaagctgaccacagaccacctttcaatctgccattctttatcttatgggagccaaactgaactacttcttctcttgaccaatggctcacggttgaaaatgcccttcccatttgtattagtctggtctcatgctgctgataaagacatacctgagactgggcaatttaccaaagaaagaggtttaatggacttacagttccacatggctggggaggcctcacaatcatggtggaaggcgaggagcaagtcacgtcttacgtggatggcagcaggcaaagagagagagcttgtgcagggaaaatcccacttataaaaccatcagatctcgtgagacttactcactatcatgagaccagcatgggaaagacctgcccccatgattcaattacctcctaccgggtttctccaacaaaacatgggaattgtgggagttacaattcaagatgagtttggttggggacacagccaacccatatgaccattcacctgtgcaagtccagatcctactcacctttcatggttcaggtccaacacagcttcttcctaaagccttttctaatccatccctccactgccagatccccaaataaggacagctttcctctgatctgccagccttatttcttttctcttttgcttcttattattatccaccttgtattttacttgtttaattataactcttatttccctacttgttataaatttgggagggaaaggacctttatatccccattagtgctgaagaagcatctgttttctaagagatcctctgaatatttgtggatgaaattcccaaatgctgacaatgccacttgatgaacttgtccctcttttggctgtaattttctctggggacctcaagtcgtctagtccctgagcacagttacccagagaaggcagccgtataaactgagcagaggctctagagcagagcagaggctctagggacaaacagccctggcattggttcccgagtctgccctcaaaagctttgggactacattgacctcactaagcctcagggccctcattcctaaagaggctaataacagtatgagaattaaccaagaaaacatgaactgccaggtcaggcacagtacccagcttgataggccttaatacatactttattttacaaggaaccagctgtccttgtaattgcctcaagtgttccactgattgtaactgtttgttttttggttttgtttttaatcagttggatctacacaagtgaaaaagacttgaatggtagtagccactggggaatcattgcaacttatagtggagctggctattatctggatttgtcaagaacaagagaggaaacagctgcacaagttgctagcctcaagaaaaatgtctggctgg

SEQ ID NO: 240 - DNA, 30140 bp, Homo sapiens
  source 1..30140: mol_type = genomic DNA
ccagtcggcggcggggacccgctgcatcgccacctccccctggaagggcagccgccccgagtggcctgggcggagaggctggttcgcgggctgcgaggtaagagcgcgcgacccgcagcggcagatgcacgaaccagaacggccggcgccggccggggccatcgcccgctgcggcagctccccgggctccatctcgcatcccctctgcgttccgcctcccttggaagcgcattccccacctccgctagtgctgccctatttccggtacccagcgcggaattccactgctcttttgttggtgcatatttattggatacctccttcttcaggatatgtcaccatagtcttttttactgaaaattagtgaaagcctaattagagtgaaagagtacatctgggttttgtttttttttttcttgtagaggaaaaaatgaacattacttgtgtaactgatggtagttgcaactgcatatttgccaatgtcacaaaatctaaaggaaaatgttatagtcacccgtggtttccttcttgcctggacactccattgtcccgggctgaaaagggtagcagtacagtgcatataatgtcaagttgtgggaggagtgtggcagattgtcattggtgcatttttttggtgatgtgtgtggttgttttgaggagtgggagctgttaagaacaccacaaatagaataaaataatatcgtgaagttatttggccgtttctaattctagacatttttctaaaaacagttgcaaaggaaagattacattgttttaaaaaaatttgaagtatgtttttaaataactaaattaatgttctttgaaattccaccaaaatgatgaagtcaccagatagcagctgataaatgtgtctgagcccagtgcgcccagctctacaaaaggcagaagaggaattttcaaatttgccagtgcccagtaagaggacatgaattttctagtaccagaggaaattctctttttacaaattttgtcagaggtatccttgggaaagtatttcatttgcttttaccctccaaattattttaatctatatttttaagagtttccattctcagttgagtttttcttgttcttctccttctgtcagttttgaaagtcttgcacaaaaacaatccaggtgttgttacagcagtgtgattaaaaccaggtcaggcctacactgaaatcccagttccaccactcattagctgtacgaccttgagcaagttacttatcctctctgatacccaatttttggattttttttttttttaaagagatgatcataacgcttaggacttgactctttgggagaattaagtgagttaagacataaaatgtgcagcatgtatttgtcatgttattagcgcttcagaaatataaatgtaaacattttggtacttcgtttatggagattcttatactagttaattttattaaaagcatgatggggaacagaagatccttttcggataacctgtgtgagtaaattaataaaacactaacactttttaagattcaaaactggattaattatgttattttacaccatttaaaatgtgcatttaaaaaatattcactgaagtgagagagaagttccttttaagttaataaataatggtaagttgcatgatccttttaactcagtttaagcatttgataacacccctaaccttgtttgaataaactcaataacagaatatagagaaataaaatatatatttcatatgagtatgtatgtaaatttatttcttttaaaggaaaatttcaggaaacaaaatgaataagctcatagtcataaaacctctagccaaagtgtgtcaatggtctgatttgtaggagagcagactcaggaatcgggaattttttttttttttttaagagtcggggtttcactctgtcactgaggctggagtgcggggaagcaaacgtagctcactgcggcctcaaactcctgggctcaagctgccctcccacctcagcttcccaagtagttgggattacaggtgcaagctgctgctcctagctgaaaagttttaattatataaaatgtttaaataaattgttattcctttcttttatgaagaaatataattgatattcttgtcacttattaaataagcaacattttaaaatgtttagcacctactgtgaagatagcactgtgctagctgctatgaataaactagaatagcatatcactgttttcctatggacaaaaacccagaaatgtaaaataaatagccatgaatgggttgagattccccctgccccctttggatactgatgacagaaatcttatatgcactatgtaagcagtgccctaggatcagaacagaaagtaacttgcaatttttaaaggtgggagaaatgactgaagtttggagtggtcaggagtttccctggaagtctcttgcctttgtgactttgtatagctccctcaaactacctgagcctgagctcctcatttataaaatggaagaattgaacctagcttccaaggtcctttctagcttcaccatgctctggtctgttgtttaatgacatgaatccaagatggacaacaaatggtcgattttgctccttctacagtaaatagatctatcttcttagcagaagtaaaatagtaaagaaagaagacatgtttgaggcctgttgaggcctgtttgctgtatgcattgtatctaatcaaggaattgagaatgtagccctaaatattaggaaggagttgaaagtttctgagcaggaacagaacatgctgaaaggaaattgtgtcagcagcatggtgtacaggagagcttggcagagagaaaggaggcaagaagacctgtgggaggcagccagtaaggaggtgaagagggcctggaccaaaggaagccaaggatgaccgaaagatttaaagatgaagcccagatttaaaagtatcctcaagtatttgtttattcttttcttagcaaattctttttagtacaaagataaaatatggcactctgagtcataaaattttctgaatttcagaagttgaatgtttttctgaatgtatcaacctgttaaagtcagttcctgtttgttattttaggcgtatattcttgggcttttttttttttttcctagagaaactatgaagtactagctgtgcaagtcagggtgggctaagctgctaaaacagatacctccctccccttcaataccctgaagaacaagaagttaattttttgctcgtgtaacattttagagagggtattccaggttgaatacctggaataatgaatgaaataataagtcgactcttatttctcctaaaataattggatcttgacattgaaattggtgtgctgattgtatttaataagtgacctccaatgcagttatttcattttgccatactttatgtaatttttattttttctgccttccctttcgatgtctaaagggaagcataattagttattggaaaggttattgagacttaaaaaaagatttgaacatggctttggttgatgctaccacacagaaactgcaaacatccaactcaccgctcttagttgctgttcatgatagtctatgcacagtgactgatgaatttagcctgtcttgaggcttcagaacttagtcttatgtcatcacgagcagagctttatcctaaattacaggttttcaatgggggggggggggaggggcagttttgcccctcaggggacatttggcaatctctggagacatttttgattgtcatacctggggggatgcccctggcatctcctgggttgaggttaaggatgctgctaatctacaatgcacagtacagccaccccacacacacacactacaaagcattctcccacccaaattgccaggagtgaggactttgagaaaccctgccctagactgttaaattcaaaaggaaataatggtttattgctcacagtgtgccaggcactgtgttaaactccttatattcatagttttgtttttatcctcacaacaacctgtgaagaaagaactctcatccgtcaccactttactgttgagggcactaagccttctaaaggttaaatgacttgcccagggccgcaactattagatgttgcagtcaggatttaattccaggcactttgtgttcaaagtgtttctcatccactgtgctatatgccagtagtgcccaaacctaactttagccagcaattgtctgcatctcttcagtttatgaacatttatttattaggacatgcaggataatcataccaacacagtccgtgtatccagaattctaattgatctagggagtgggagagcctgcctctctattttttttaaattggtgtgaaatatacataacataaaattggccattttaaccattttaagtatataattgagtgacattaggtagatttattatattatgtaaatggacttaacacaatttatttccagaacttttttatcatcccaaacagaaactctgtactcattaaacagtaactccctgtttccccactcccccttgccccagacgctagtaacctccattctacttcccctttctgtgagtctccctgttgtagctacctgatgtaagtgaaatcagaccatgtttgatcttttgtgtctggcttattcacttagcatagtattttcaaggtttatccatgcgtgcaaattcccttcttttttatgacgaaatactatttaattgtgtgtgtacatgtgcacacacaatgaaatactatttcatcataaaaaggaagtttgtacacatatatacacacacaccaaattttgtttatccattcatcagttcatgggcatttgcggtttttttccacctttcgactgttgtgactttccgtttatttttaatctgaactttactccatcacttcctccctttccttttttattcgcaccataattttgaacaagcagactttgtattttcattactctggggatttttttgagggaggcttgctaccctttggggttgtggtaaggttgtgccagtaacagaattcatgcagtaaaaaacattcatgggactttcttttgtgataggtactagggatgcagagatgaataatacaaggtctcgaccttcaaggagctcagggtttagtagggaaaacagaggtataagtaagtcattgcaatgccaggtcccagatatgggaggattccagcaaagagaggataaggccaggcacagccgctcatgcctatagtcccaacactttgggaagctgagatgggaggatcacttaagcccagcagttcaaggctaccctgggcaacatagtgagtggcaaaaaatacaaaaattagccaggcagggtggcacatgcctgtggtcccagttatttgggagtttgaggtgggagaatcacctgagcctgggagattgaggctgcagtgagctgtgatcacgccactgcactccagcctgggtgacacagtgagaggtgagagcctgtatctaaaaaaaaaaaaaagagaggatcagatttacctggagaggtcagagaaggtttccagagggagtaaaacttgagcttcattttgaagaatgagaggggaatacaaggagggaaaaataataggaacaagacctgttcctgttacagcaaacctgtaactcccaccatggaatgggcacgttttcctgtagacatttggagccagtgtgggcttcaagcctagggggctgatatgatcatttgtcaatctgaagatgactcttggaacaacgtagaggatagggtgaggtgggtaggctggtggctgacagactaggtcaggagagggctgaccaagaggaggaggggtgacgagggctggaactaaggcatatgagctgggatggaaagaagctagatagaaaggaaacaaaaaccatcagaactgtggaaccaactccatgggagggataagaagagggcctattctagggtgaaacccaaatttctggcatggtaccattaacccaaacagaaagaggagatttccagagaaagaaaagcaaattttgcgggaggtgagtgtgagctgtctagagaatatgcaggtagaactacatgttaactgtaacatatacttgtctgcacctcaggagatggctgattaagaattcaggattcaggccaggcacggtggcacacgcctttaatcccagctgcttaagagactggggcgggaggattgcttgagcctgggaatccgagatcagcctgggcaacatagcaagacactgtctctaaaaaaaaaaaaaaattcaagattctggagtcaatattacttaaggtagcaactgtctattctatagaaaatggacaaatatagataaaaagccacttccctttctaaaagtgtccctgcaattcaagtgaatactaggaaggattgtgttcattcttctaaacaagactcacatgtatcttagcacaaaaagaggattcttttatgatacaaatgcactgagaatttggtcaggctatcacaatgaactgatagttcagatggatttgagtctttataccactctggaatctggaccaactgggcctcctaaggccattttgcagatctgggcttgtttctgaagctacagacaggcctcttccaagcactctaagtgctccacaaataatatttctgtttcccagaacaaccccacaaaaaggtactcttactccatttttagatgaggaagtggaggctcatgatgtcaggtaagctttctcagctcccaagtggttaagccctcagtttaatgtcatttgactccagagccctatgttgcaccatgccttgataataggccatatgggtttcatgtatttcagatggggaaggttagtgtgaggtgaaagatacacaattaaccttttaaccatggaactgaaatatttacagatgaagtgatacaatagctggaattaattccaaaataattggggtggtactggtccatggcctgttaggaactgggccgcagagcaggaggtgagcagcaggctagtgagcattgctaccccctatcatatcagcagtggcatcagatgggtgaggatgtaaatgaactaggattggcattgagttgatattgttggatctaggtgatagatttatcattataatattctctctttgtgatatttctgatattttctaaaataaaaagttgtagttatttatttatttagacggagtttcactcttttgcccaggttggagtgcagtggtgtgatctcggctcactgtagcctccgcctcccgggttcaagcgattctcctgcctcagcctcccgagtagctgggactacagcctcccgagtagctcaccaccacacccagctaacttttgtatttttagtagagaggaggtttcaccatgttagataggctggtctcaaactcctgacctcaggtgatccacccacctcggcctcccagagtgctgggattacaggcgtgagccagtgtgcccagccaataaaaagtttttgaaaggatttagataaaatagttggggaaatggcatttttgttcaaagccaattatttatgtttggaatatcttttgtgcttggagttctccattacagagttccccagtgttcctattaataagtaacattgagcagaggaatgcactgtttagatcagcagtccccaacctttttggcaccagggaccggcttcatggaagacaatttttccacagacctggggtaggaagtgtttggggatgaaactgttccacctcagatcatcaggcattagtgagattctcataaggagcaggcaatctagattcttcacatgcgcagttcacaatagggttctcactcctgtgagaatctaatgccacccctgatctgacaggaggcggagctcgggcggtaatgctcacttgcctgcggctcacctcctgctgtggggcccggttcctaacaggccatggaccagtacccgtctgcagcctggggactggggaccctgctttagatgatgtactctggctttgcattctggcattagctaagcaccctcttaaaggaaattgggtctatactctcagtccgtgttctccctaacacctggaaacattgaataccttcaatgctgggaagttaactcccaccacaactagaacagctatgggaaagacaacagttgattttgaagagtgtcaccaatttgcacatgattccatccttaaccattcttatcctatcagctctgccaaacatggagaatagttggctgcaggacagctatttttcctacttgtagatgcaactatttctcacccaccaggatgtaaaaggtccctgtaccctaagattggtcctacatacacacccaatgggaaaatgagatgaaaaatttaaagcagtaaatatttgaggaagtagatagagtaatttagaaaaagaaaatacacagggccaagcacagtggctcacatctgtaatcccagcactttgagaggccaaggtgagaggattgcttgagctcaggagtttgaggccagcctaggcaatgtagtgagaccccacctctacaaaaaattaaaaacttagttgggcttggtagtatgtacctgtagtttaagaaacttgggaggctgagctgaggcaggaggattgtttgagcccgggtggtcaaggctgcagtgggccatgattgtgccagagtactccagcctgggtgatagagtgagactctgtctcaaacaaaaaaaaaaacagagacagaaaaaaagaaagaaaatatatggatgtatatcatataaaaatataaataagggaggccaagtgcagtggcatgcctgtaatcccagcactttgggaggctgaagcaggaggatcacttgaggccgagaattcgagaccagcctgggcaacgtattgagacctcatctctgcaaaaaatcaaaaaatgaggcggaaggatggcttgagcccaggagatcaagccttcagtgagctgtgatcgtaccactacactccaggctgggtaacagagagagaccctgcctcaagataaataaattcatacatacatacatacatacgtacatacatacatacataagaagacttgtttctttccatttgcaatgtttcattcaaaggctagaattaaattgccgtaggccatcacaagtttagcttgaatattattattttttcaagatggagtctcactctgtcacccaggctggagtgcagtggtgtgattttggctcactgcaacctctgtctcccgggttcaaccgattcttctgcctcagcctcccatgtagctgggattacaggcgcccgccaccacacctggctaagttttgtatttttagtagagacagggtttcaccatgttggccagactggtcttgaactcctgacctcaagtgatctacccgcctcaccctcccaaagtgctgggattataggcttgagccactgcacccagcctagcttgaagaaaatttgataggagtttgtttttttctatttataggccaagcaataccacgtataaatattaagaatcatggctgttccttagtgcctagttgtttataaaccatgggaaagaatgaaatcattgaccaaatgagacagggtaacagtcttccctgggagtaaagagactagcagtctctattgacatatattttaggcctggcctccaaaataaatttacccaaagaagtgatgtatttgtgtccagggctaccgagcctaatctttggctgcctctgtgttattctaagttgtaattttccatgtcatctaaatttgtaataattctttaatatgatagtgtttcagtgaacaaacattctgcttgctgcatttcttctgagtgagtaattccctgacaactaccagatcttggcagaagcaaagttggtcataagttatgctccactctcagtgctggtgaaacgtatgatgcgtaacacagtgtttttgaattcagtgctgattttcctaaaggacatttggaaggaaaaaagaaaaggaaaggaaatacccaaattcaggaatagaacttacatattattataagacttaaaaaatacatgaacactaatgatgaactcatttcttcgaagtaaaaggccttatgctattttttcccatttccctatgtggcttgattgtggcgaaagtggctgtgtgagtttccattattgaaggagttaaggtctgtggaatcaaagcatgagacaacatgcaaggaccaggttggtttccatttaacagccaacctaatctaactgaaaggattgagaggtttgtcttttttggaaagtgttaaggttcttccaagtaaccagcaatgtgactttaccacactgttcattaacggggctttggatggccaccatgtctgctgctggctgagtccaaaactgcggtcactctctccagcaagctcatggagggttacgtcatcttcactcagccagcccaacgctttttccatctgctaaaatgtagaacatggttgttgattctactctttctacaaagaaagagagggaacatggatgagttgctgcttttaaagattatatgttaattgctgttttaaaaatctgctcagctaagcacgcttagtgtaatcagtcaccatggagttttttaataggacagcttctgctcttacagagcagagtttttatgccagtaggttagaagcataacatgtttctatattgaagtgattctccaacaaggacttcttttcatcaggggtgacctaaaagttattacttgaaatataattacatcatgttcttaactgggtttagtagtaggataattacaaggttgtatccacctcaaataggaagaactgataagttttgcacaattatttaactcctctgttaggccttccttatttcctgttctctttttaaaatttgactaagatattgctaatgggcttgggagcctataaaatgatcagaattgttgccttatgttttgcatgtttggggtaacattggagccagatgtactcttaaataaaaggtaggcctacaaaaccagtttctcagttgcattcaaaatgtgattaaaaaaaaaaaaaggagaatctcccttataggtgaactttttaatttgtgctttattttcccttttgccattcatgagatttcttaaataaaatgatatcttttcttttcttcattattattttaaaggtctctggggaacaagactcatggaggaaagcagcactaaccgagagaaataccttaaaagtgttttacgggaactggtcacatacctcctttttctcatagtcttgtgcatctgtaagtagaatatttccttgcactaatgggaaagttttgaaaagatttgacctatccaaatcataattaaaaggaagtgtgtatgcaccagaggggcaactgggaagttaccttcttacctttgtttttaattctaatatttttatttgggcatttgtttattgactatcttcctatggtagaatgcaagctttataagagaagggacgtgatttgttctctgctgtacccccatttcccaaaactgcagatggcaacagaaggctctgaaaaatatataagaaagaatttttctaattgtgactaaattgtgaccaaatgctaagtgactgtggacttgcgtttaacacaggacgggagaggcaaagagttcaattccaatttagaatttggtcaagttctcttctgcactctggtaaacattaattaaaaatcagcattatctgaccagccagttcatcgtcagtggtggtgattttcactatgagatacgcgtggcaacttgccagacaccaagaaaccaagttagaggatttttgtattagattccttaacaatgaatacagtatcaccattattacagtatcatcattattgtcatactattattatatcagttaacataaagtctgcataagaattgtttccagaaaaatgactttccaaatttaactttcaggaaatacaaataatgctactaatattgcttttattggcgtatacatgtaatatccccttcttttggatttggatatgttgtgtcattgcctcattttaattcattatttcttctcaatctttaataattgctggacttttactccacaagaaacttgctataggcccatctctttcgtcttctttccttctttcagttcgtcttcccatcctctggtagggggaggggagggatgcctgagcgagagactagctgtaggaaccatttgtctcaaagtccagaaagccacaggtgatggatttgtcctctgaatcaaagggcgttcgatgatggatttctgtcatgtctcatctaaagtcttcacgagaacagatgaggaagcagttttatgaccccagagcctcctaccaaactcctctgagaaaaggtttccttttttttttttttttttaaattagagacagagtcttgatctgtttcacaggccggagtgcagtggcacaatcatagctcactgcagcctcgagctcctaggcttcagtgatcctcccacctcagtcacccacgtagctgggactacagctgcacaccaccatgcccagctaacttttaaaacatttttgtagaggtggggtctcactttgctgcctatactggtctccagctcctagcttcaagtgatcctcctgccttggaagttctgggattataggcatgagccactgcacccagcctggatgtgatatttttatgttttaaattgttagagtttagaaacttgagattgagtttgctgcctgcattaaaatgatgcttaaacattaaactgcagtggccttaaatattaacaagttgattagaattactaagttcttttcaagctttacatatacagacaaatttcttatgcaaaatagaaggtaacccctgtacgtaagtctagaatttcagcagtccccaaaactgactgagcattagaatcactctgattttaaaatacatatgtggttttccgagatctactaacagagcctccataatgtagcctagagaaacgagttttcagatgtagtgataaatttggaaggtaagtcagagaagtaagctgaagacagagttttaggaaatatgcctaaagtcacataatgaattggttttcttgtttatttgagaatattgtcgctttttgttctttttcatgcaaatcacattttatttcttatgtgagtagctatatatttaaaaattttgtttttggaatattgtagaatctctacttaagaaagtatcttagcagtcatatggtctgacctcactgaatgctaaattctctttaaaacatccgtctcagatggttactcatactgcctctgattgaacaagagtcccaggttaagggacttacttctttgaaatcgttcatttcatttttctacagctatgttagaaagttcgtccttagcagtgaagccagagtctatctcttataacttctacccagttgcaccctccaagcctacctataccaagtatcttttttccacgttgcttttccaattcagctcttcgcaagtttggagacaaatacctaatctcctctaagccttctccaggttaagcctttccagttcatccagctgttgattatgtgattggagacacaagtttgagtaactcccatgatggaaagtccctctggtgaatgttctgttcatcagagtccctaagaaagcacatgagcctcaccatggtgagtggggccatgagattcctaaaccagacactacactgtggcttgtgcaacctaccatggccagactcatgaggctgtttcatcatataaataattccttagtctcttcaccaaaaactgtgaagcactgtgtcccccagctgtatgtgagctagcctgggaccatagtggaggactcctctttcaaccctgttaaatttcatcttgttaggatctgcccatttttccattctgttgaaattatcttggaactggattcattcatctcacatcagctactccccgaagcctcacagtatcagcagagttattatctctatccatatccttgtaaattattaaactaaaaaagattggtctaagaacatcagtccattaatcaaggctctttggttggagttcacttcattatattatcatccagctcagagtgttcataaacaagatgataggaaagacttttccaaatgcctgtctgagtaattcccccattcctgtgatctgtgagctgttgaccagattaaaaaggaagtgagaataaccaggcaagatttactcctagcaaaaccttactggcttctagtgactgagtcctttccctattgcttaccagctatctttttaattactagttttaaaatcttgccagcaatgtcaatatcaaatgaccaagaatatcaaactcattactctgtatgtaaatgagatagtgtacttacccctatggcttaagtattaggctgttcttgcattgccctaaatacctgagactgggtaatttataaaaaaagaggtttggccaggcactgtggatcaggcctgtaatctcagcactttgtcaggctgaagcaggtgtaatggtgagccaagagttcaaacttagcctggacaacaaggtgaaaccccctctctgcaaaaaatacaaaaattatctgggcatggtggcatgcacctgtagtcccagccacccaggaggctgaggtgggaaaattgtttgaagctgggagtcagtgattgcagtgagccatgattgcaacactgcactccatccagcctgggcgacagagcaagaccctgtctcaaaaaataaataaatgaataaataaataaaataaataaataaatagaaaagaaaaagaaagaaaagagatttatttgcctcatggttctgcaggctgtaagggaagcatagctccagcatctgcttctggggaggcctcaggaagctgttactcatggcagaaggtgaagcaggagcttgcacatcatgtggcaaaagcaggagcaagagagagagaatggggcagggaagaagccccacacttttaaatgaccagatcgcatgagaaatcactcgttacctcaaggacagtaccaagaggatggtactaaattcctgagaaatccacccccatgatctgatcacctcgtaccaggccccgccttcagcattggggattatgtttcaacatgagatttggatggggacaacatccaaactatatcaccttgcatagtaggtagggttttaaaaagcagtttggcacagtaagaaaagtacagattttttttgcatcagacagacctgagttaaaatcccagcttcactgctaacatgctaggtaaatgtgggcaagttaattaacatttctaagcctttgtttcctcactggtaaaacaagtatttggaaatatcattgtgaagattagaaataatacatgaaaagatcctaggatgctgtctgtcatacagtagtagtagtaagaagttattcttgccaaagattgttgagaatggcagaattatctcagttctaagagctatagtttctaattatttgagcctagactcagattcatttggagcagctaactgctcaccaagagcttattttccatcttaccaatgaggttatgtgccctgtgtttttaaaatcagtctacttaaccaagagaacagaaatgacatgagaattaagtaatctcactttctctgttatttaggatttattcctactcaaaacctgagagttgctatgaattcaccattaaagcacttattaatatacatgggttactgttataaatagcaatagtattgctattgtgtgagttaggtgttgaagttcaagaaaggaataaagaatatttagaagatctttgaaaacagtgtctgggtacggtggctcatgcctgtaatctcagcactttgggaggccgaggcaggcagatcacttgaggtcacgagttcaagaccagcctgggcaacttggcgagacctcgtctctacaagatatacaaaaattagccgggtatgttggcatgcacctgtaatcccagctacttaggaggctgaagcacaagaatcacttgaacctgggaagcagaggttgcagtgagccaagattgtaccactgcactccagcctgggcaatagagcaacactctgtctcgaaaaaaaaaaaaaaaaaaaaaaaaagaaagaaagaaggaaggaaggaaagaaaaaaaaaggaaaaaatgcaaggaaggtatttggtgaatctataataataaaaatgtatttgtcatttcctttttctgtgctctcattctataaaattgagtaaaaaatctatatatagtttaaacacattaatagaaatcacaaaagttagctgagtcaacattgtagaaacataatatttctgtatgtcaaagaaatagacaacattaaaaagcagaaagcaattacaaagaatgattataagaaacatcacaaagggttaatattttaacacatttgaaactcaaaaatcactgagaaaagcagtagacttccataaatattttatagagtagaaaaaataggccaagcacagtggctcatgcctgtaattccagcactttgggaggccgaggagggtggatcacgaggtcaggagttcaagaccagcccggccaagatggtgaaaccccatctctactaaaaatacaaaaactagccaggcgtggtggcaggtgcctgtaatcccagctacttgggaggctgaggcagggaattgcttaaacccttaaacccgggaggtggaggttgcagtgagccaagttcgcaccactgcattccagcctgggcgacagaacgagactctgtctcagaaaaagaaaagaaaagaatagaaaaagaatccatgggcaggcacagtggctcatgcttataatcccagtactctaggaagccaaggtgagaggatcaattgaggccaggagttcaaggccagcctgggcaacatagcaagactttgtctctattaaaaattttaaaattagccaggcatggtgacgcacacctgtagtcccaattacttgggagcctgaggcaggagaactgcttgaggctgcagtgagctatgattagaccactgcactccagcctgagctacacagtgagaccttgtgtcaaaaaagtaaaaaaataaaaattagccaggcatggtggcacatgcctgtagtcccagctactcaggaggctgaggcaagaggatgacttgagtctggaagatggagactgcagtgagctgtggtcatgccactgcactccagcctgggtgacagagcaagaccctgtctcaaaaaaaaaaaaaagaaaagaaaagaaaaataaataaaatttattcaaatacaaaagtgatgtggtttgactctgtgttgccacccagatctcatctccaattgtaatccccgtgtattgacggaggttcctggtaggagatgattggatcatggggatggtttcccctctgctgttctcatgatagtgagtgagttctcatgaaatctggttgtttggtaggtgtctgtcacttaccccttctttttctctctcctgctgccttgtgaagaaggtacttccttctcctttgccttccaccatgattataagtttcctgaggccttcccagccatttggaactgtaagtcaattaaacctctttcctttataaattaccgagtctcaggcagttttttatagaagtgtgaaaatggtctaatacagagacttggtaccaggagtggggtactgctataaaaaataacctgaagatatggaagcgactctggaactgggtaacaggcagcaattggaacagtttggagggctcagaagaagacaggaagatgtgggaaagtttggaatttcctagagacttgttgaatggctttgaccaatacactgatagtgatatggacaatgaagtccaggctgagatggtctcaggtggagatgaggaacttattgggaactggagtaaacgtcactcttacatgttttagcgaagagactggcagcatttttcccctgccctagagatctgtggaactttgaacttgagagacatgatttagagtatctggcagaagatatttctaagcaccaaagcattcgagaggtgacctggcttttcctgaaagcatacagttatatgtgctcacaaagagatggtttgaaattggaacttatgtttaaaggggaagcagagtgcaacaaaagtttagggagtttgcagcctgaccatgtggtagaaaagaaaaacccattttctggggagaaattcaagctggctggagaaatttgcataagtaacgaggagctgaatgtgagttgccaagacaatggggtaaatgtctccagggcgtttcagaaaatcttcagggcagaccctcacaacacaagcctggaggcctagaagggaaaaatggtgtgagccaggcccaggcccaggccccagctgttctgtgcagccttgggacatggcaccctgtgttccagccactccagctccagctgtggttaaaaggagccaaggtacagctggaccattgcttcagagggtacaaatcccaagcattagcagcttccatgtggtgttgggtctttgggtgcacagaagacaaaagttgagctttggaagccgctgcctagatttcagaggatgtatggaaacacctcgatgtccaggcagaagtctgctgcaggggcagagccttatggagaacctctgctagggcaatgcaggggggaaatgtggggttggagctcccacacagagtccccactggggcactgcctcatggagctgtgagaaaaggaccaccatcctccagactccagaatggtagatccaccaacagattgcactctgcgcttagaaaagctgcaggcactcaatgccagcctgtgaaagcagctgcaggggctgtaccctgcagagccacagaggtggagctgtccaaggccatgggagcccaccccttgcattagcatggagacaggggatcaaaggagattttggagatctaagatttaatgaatgccctgtcgagtttcagacttgaatggggcctgtgacccctttgttttggccaatttctcctatttggaatgggaacatatacccaatgcctgtacccccattgtatcttggaagtaactaacttgcttttgattttacagactcaggcagaagggacttgccttgtctcagatgagactttggacttgaacttttgagttaatgttggaacgaattaagacattggggttctgttgggaaggcgtatttggttttgaaatgtgagaaggacatgagattttggaggggccaggggtagaatgatatggtttgactctgtgtctccacccaaatctcatctccaattgtaatccccatgtgtcaagggagggacctgatgggaggtgactgaatcataggggcagtttcccccatgctgtttgcatgatagtgagggagttctcatgagatctggttttttggtaagtgtctgggcttcccccttttccctctctctcctactgccttgtgaagaaggtacttgcttctcctttgccttctgccatgattgtaagtttcctgaggtctccccagccattcagaactgtgagtcaattaaacctcttcctgcctattctcaggcagttctttatagcagtatgaaaatggactactacagaaagtgtgtaactttaaactcagtagtatccaaagaagtaatgaaaatggagaaacgaacaacaaaatcatagtacaatatggtgtatgtactaggacaggaagagcccttttaagaagagatctatgtatttccatttgtttatctctgaaagaaagcaactttgccttgtattctgaaaaagaaaggaatattttattttacttgtaaaaatcttacaaggatgctagtctaaatatagttttcctaatttgccagagaatccatgaagatcgagttgataacaagatcagtgaagtaaaggtcagtgagttaatctcacagcagctgcaggctaattccatttccagtgaaaaacgtcttgattgctcaccacatatcttttcaccacaaacagtttcagtcttaagatcacatgttgcaatccatgagaagtaactattaagccttcaactatgactggagggctcctcgccctttctgataaattgactggacaaaaactcaattttaaaatgacaagaaatagaagatgtataaatgtactttaaatgtgaccaaaatgggttgtgaaaacacaagacacaatatccaaaaatgctggcaacacagtacactgtagagtattggttgtttatttacccttgctattgtgtggctgagcttactgccactgcccagcattgcaagggcatcaaactgcctatcactagcctaggaaaagatcaaaattcaaaattctaagtacagtttctactgaatgcttatcacttttgcaccattttaaagtaaaaaaatcagtaagttgaaccatcatatatccaagattgtctgtatataaatattatacatctttctctcacttttaaaacaaaataatactagccaatactaccattctcaaaagcacttgtgtcaacagcctttaccccttaaagattttcctcacaattttaaaattgttacttactattttctttgaaatgttgaccaaacctggattaaaagatttgggggttttagtgactgtatttcacaaactctcttattgattctgcagcctcacttctgcctcctaaaaagccctcaccaaggtcacgggggatggctcttttcagcctcttcctggcatttggtccagtggcatttggcattctaggacttccctctttgtctttgataactccctctcttcctgtgttcctccttgctgtgttcacttgcttcgctttcttctttctgaagcatgtttacacagtgttttctctgattgggcctgtgacgttctttaggtcatcttttccacaaataatgcttcaactagtacttgcgtgccagtgactccacgtcccactcatgagctctgaacctagtaccagcttctgctggacatctacaatgggatctctcacaggcctctctcattggtaacatgccccagcctgaactcatctcccacccatctatccagccatgctctctagttcacctgaacacttgggtgtcatcctagatgctttcccttcccagtcttctgtgatcattctgcctcatcagaggctctctaatctgtcttctttcctatatcgctcttgtccctattttaatcctaatcatctatttcctgacttattcattccttaagttggtcagtaatttaattaaaaacagatttaggccctgaccttaaatgtgataagtgatatgaaaggagatgactggggaaaaggatttccctcaaggaaggcctctgtgaagcctgaagcaagaatgaaaacgagtcagacgaagagagaattgtatgaatgaaggctctgaggcaggaaaacactcagatcattccagaatcacttagaagccaagtgaagccagttcctggagagcagatcatcaaatgaagatggaaaggtgaccaggggccagacctgtagttttggtgggccttggtgagggatttacagtaggacaccccatggtttaagtatgaaagtgacaagattcctttaagttttaagaggcctcgaaatatgaaccacagattagatggaagctactctccctgtgtctggactttttagaatttccaagagctgctgtttctggaaccagattaatacaagtcagtcttccatttatttatttatgtatttatttgagacagggtctcactctgtcacccaggctggggtgcagtggcatgaacacagctcactgcagcttgggggctcaagagatcctcctgcctcagcctcccatgtagttgggaccacaggcacctaccacccagctaattttatttgttgtagaaatgaggtctcattttgctgcccaggctgttcttgaactcttgggctcaagcgatcctcctgcaacatcttcccaaagtgctgggatcactcttccatttaacatgctatctcaacgtcaagataaactttaaaatctttagataataggctggcattttacttaaacgatctttacttcttcagaactgccattccctataaatatctggttcttcaaccacatcaaaccacttgtgatctcaaaaagcctcagcgtacactgtcctttctgtcattctaattcctcctcatccttcaaaatcaactcaaggaccagatccagggagaagcttagtggtgcccacccgaaccggccccctccttcgagttgtgctgccattcgggcccacctcttcacacagggttgtcagaccagaccagctcatgcgttcaccgcccttgcagggatgggatgcagctgtgcacctctcagtgccgacacctggagagtctcacgaaatgttgacaacatggcctgtttccatttcttgttcactaggacttctcatttactaacacacagaatttcctgtagtatgtccacttaatcagttcaagcctaataattccttgatttgggtatagtgctttgcatttatatactgatggtccccaacttacgatggttcgatttatgatttttcaacttcatggtgatgtgaaagtgatacacattctatagaaaccacactttcaattttgaattttggtctttttccaggctaccatactctaaagatagagccacagatcccagtcagccatgtgattatgagggtaagcgaccaatactctacagtgtattgtattgccagatggttttgcccaactagcctaatgtaagtattctaaacatgtttaaggtaggccaggctaagctgtgttgctcattcagtaggttaggtatattaaatgcattttcaacttatgatattttcaatttacaatgagtttatcaggatgtaactctactataagtcaaggatcatcttgtatagcacttttaatttatagagtcccttcaaatgtttgtttgtattttatttccactgcatccctgtgaggataccataagttatacagctaacaaaacagttagttttcctgtgcaaagtgatggcttcatcttgtggcagattacctggaatactgtggccaaggcatcttagttctactgtctttatatatctagtacagttatatttttatggcagctctgatttcttctttggcccaagggttattaagagagggaaaaaatttaatttcttaacagatatatatatctatgtcaagtcatatatttaattcaaacccttaatattcctaggtaatttttgtctactttctctgtcaaagattgaaagatacagggttttaagtttccaactgtaattgtagttttggtaattgttttattactaacaggtattgctctgtatattttgatgttctgttattcaatacataagaattcattacagttgaatcatcatgtatagtataatttaccaatgtaaaataacctttttatcaaactgagtattcaccttataatgtcctctgtcagaggattataataccacttaaaaccttttaaaaaatattttgttcctgataattttagctttgagtggttttcttataacaattctagagatgcgtttttattttttaaccaagatttaaagctttgtgatttagtaagagtctaaaccattcacagttcatgcttttttgtcaaatttctattatttagtattttcctctcttttatattttcctgttattttccatttctattcttttgttagactggaaattttttctttgctttcttttatcctagtgatttgaaatttatgtaatatatactattctacaataccctttatttgttttcaatatttgaacctatatttttcaacattattaagaataaaatagtatttgttgctattttgaaatgatagaccatgtttttaaggcaggttggtggttgttaaggcaccagtatcggccaggcacgatggctcacacctgtaatctcagcactttcggaggccgaggtgggcagatcgtttgagcccagcactttggccgatactgtggtttactgtattttgttctagtttattttatggaaaatgggaattcagtggttaagacaaggattaaatagcagaagaaagatgtgtacatatgtacagatgtatgtgtcctttatatgttttttagtactcttgtctcctttctggtcctcatttaaggttatctatttcatgcagtaaatttttcttcacaattcatttcatttagagagtgaatgctaccttccaagtgggctttctccagttttcctttcagggacttaaaggagaagtgatgttaacagttttatatttccattgcattttacagtgtgcagatgtcttcacatatatttccccatttgagctttacaaaagcccttagtattattctcattgtctagattccaaaatcaggcttagaggagttaaatagttgtccaggatctcaagatgcaagacccacaatcatgaacagaggcagatgttcaggatggaggcaagctgaaactcaaaaccaaatcattatgactccaaattcaggagtcttttagctgccacctgcatgggctcttggtgtagctgaccaccagagtttgtagagctgtcattcaggtgtgccatggactttcctgggacctggcacaggagaaggactgagttaatgtttgctgattaaatatctgttacaggctgggcgcggtggctcacgaccgtaatcccagcactttgggaggccgagcagggaggatcacttgagctcacaagtttgagaccagcctgggcagcatggcgaaaccccgtctctacaaaaaatttgaaaattagctggccatggtgatgcatgcctgtagtcccaggtactcaggaagctgaggtgggaggatcacatgagcccatgagattgaagctgcagtgagctgagatggtgccactgcactccagccttggccatagagccagaccttatctcaaaaaaaaaaaaaaaaagttacaataatcttcccttcaaagctggaaggcattatttacctgtctgtccagcagatggtgctacataaccaagggaatctgttgcttgcccttggtgaagctattaaagccaatacagatcttgagaatttcaaaagcaaaaatcaatactggattatgagtgctctaggaaaataaagagataaattttcaatttacatacttatatatagttataccatatttgtaaataaaaatatataaataatttatcaaaattcctttttaacagcaacaaccacagtaaacccacaggttaaaaactccacaacagtctatattaatcagtcaatgcaaagtacattccaattccaagttaactgaaaataatcaacttaatcatttggttggctctgagcagccttcactgcttgctcttgtgtcatgtttctttctgtcctcgattggctatagacttacagacggcttttgcagaggacagtgtactcatgtccatcctttgcatcctttgtgggagttggctaggcagcactctccctggggacactatgaactcctcttcttgagtgcagagatcacgtcttgttcatcttcatgcccaataccttgttccataaatatgaatggattagaattctaaactcttaactctgccccaagacagttctgagaggtagtaagtcatataacacctgaagaggactgttcttgtcctaattacattaggttataagatgacaggtgagggagccaaaccagggggcctggaaattattcatacatctctagatacagtatacaagttgtgtgtattatgtgtatttactctgtaattgattgcttgagatgaacccccaaacacactcgtgtttggatcattattatctacccttctccttaaataatcttaatttcctatgatgcttgaaagggaaagaggggccaggtgtggtggttcacacctgtaatcccagcactttgggaggctgaggtgggagcatcacctgaggtctggagttcaagaccaacctgaccaacatggtgaaaccctgtctctactaaaaataaaaaatcagctgggcatggtagcacatgcctgtaatcccagctacttgggaggctgaagtgggagaatcgcttgaacctgggagggggaggttgcagtgagccgagatcactccattgcactccagactgggcaacaacagtgaaactccgtctcaaaaaaaaaaaaaaaaaaaaggcagagtggggaagagagctgcatgaaggagagatttactaaatagtacttaatcccaaaataatttctataggtttgaatatgatccctgaaatttattataggttcaggtaagtattaatcacgggtattcagaactgtggtttaaaaaatgtatagaacatgtttccttccccttgaaactttttatcagctaattataggaattatattatacctgcaatcattaaagtccagaatgagacagtacttggtaaagtgctgaaatttataataaatgcattatagcaatccagttaaggaggaagagccaccattattgaacatttgttaagtgtcagccattgtactagataaattttagttattatttttatttaggcaccaaaaaatccatgggatagttggttatccccatcttactgaagaggaaactgaagctcagaaagtttaagcaacttgcacaggtcacatagcaagtaaggagcatggccaggaatcagaccctgatctcctttggtctactaagcttgcaaaggatcttcccgcctccttccaagaccattcaatattatcagtaaatgtccatggcaaggatgtagttcgagttatagggttccattcaagatatgattggtaggtgggaagcagatatgtctgtgtcaatcagtatcctggaagaaggagatgatgaactcaagtggtgattaagggaagtttaatgaagggactatttacagagatgtggtggggttaagagaaccaacaaggggaagtgatgcactcaaaaagttactacctccaggctttaggggattgggggagggagtggcacagtgtgaacccagtgtggttgtgagaaaagggattccctcagcagccatggccaaggttagagtctccactgccaaactgcatccaggtggtgagggaatggagaataggggtggggtaacaaactctgacctcggtatccccaaagggcaaaggattccaggtggtacagttcgtaaagattagcctcagggcacagaacagggcagagaagaatggagaattgatctggaggaaacaaacaatggcttgcccatgttattgcagggagagtaggctggtgtgcacagcaggagggtggggagcccagcatatagctgtgttggggcctgtgcagatcagcctcactggcagggaggatctgagccgagaggtggtggaagatgaaatcgagtaggcatgttggtagtcctaaatatcaagtaaacgttcctgatcttacattgatactcaatagtaagccaattttgtttcccataagccaatattaatattacgtatttcttttataagccagagatatagagagataccctagaagaatgataggggaaaggaaggcaagggtgagagaagaccttgtgtgaatttgtccaaaatgtttatccacaggaacaatccctttgtgaaggctgctggtatgtgaatgtgtgccggttcccttggggcgttcatttggatctttctgtgttccagtgacctacggcatgatgagctccaatgtgtactactacacccggatgatgtcacagctcttcctagacacccccgtgtccaaaacggagaaaactaactttaaaactctgtcttccatggaagacttctggaaggtatttgcaaataactttgaaagtacctctctatcacagaaaattgttcatttggcttcatcatttcaatgcatgagtatcgacaggacctgctttgcatttaacactgtgtgagacgtaagttatggtgagttgttagaagttactgttcctactctcaaagggggtaaactaacattgagaactttgcctgtgccttgcactgtgctgagtgtttcatatcttaccttatttaatttctatagtctaactctataaggtaagtactaagactatgccctagtttgttaatgaggaaaatgagattcaggatgtttaaatgcgtatggtcacatggctagggaacaagaaaaattgatttttttctagcctgacagctacttcatcctagtttgtaattcattccatgagtcaagattcaataaatatttattgagaatctcctagaatgtaaggccaatgaagggcagtgtggttcttctgtcttgcttcgccttttgtgttttgtctctttgttgatgatggcatgtatccccagctcttagaacagtgcttgattcaaagtaagcacattctttcaaaggtctgctgttggtggggcttggtggctcacgcctgtaatcccagcactttgggaggccaaggcaggaggattgctttagcccaggattttgaaaccagctgggcacaacatagtatgactttgtctctccaaaaaagttaaagaattagcagggtgtggtggtacacacctgcagtcccagctactcaggaggctgaggtgggagaatcacttgagcgattgcttgaggtcaaggctgcagtgagccatggccatgctactgcattccagctggggcaacagagtgagaccctttctcaaaaaaaatcccccccaaaaaaaaacccaaaaacaaacaaaaaaggtctgctgttgtgaagttcaacccaatccagccccttcccaagttgtcacaaattccaacgtagttaacagtataccaatgagtgataccacaggaaaaatattaaactgatctgagggatatggggcttggaatctaagaaaattggaagggaaattgaaaaggaaattattatttctccttggggagatagtttctaaaattcttactacaccctggggtcagagctgttgattttaaggatagagacaactgagtcacaggaaactattcatatataaaagtacctggcatccaaaaccacacttgtataatatgaatctttcaccatctgagtagggcaaatcagtctatctctgttgatcatctgacaaggatagcacactgagaaatagatctgtcttccctacaggcatagctagttgtacaaactaacaagagacttttgtatacacattccatgatgataaatgccaatcactaaagggacgaggagggattggagagttcaccatacagcaaaatagtccagacaggtgaaaggtctatcaaatgccaggctggtaatcaaaactgtagccttttctctaaacaaagtttagaaccatgattgtgtgggacattattttaataagggaaagtgcagttaatcatgaccccacctttagtccaagaacaaaaatcagagctgccacgtattaagtacccactctgtgccaggtgcagtaactatgcaaaagatgggttttccagatgcaagaaccttggttcagaggaccctgctcaaggcctcatagctaacaaatgatggggcaagatgctatcccaaatctctctgacaacaaaactcattcttatcactctactattttcatagagttgccaaatgcttggttatgcaaacgatgcaggcaggggcaagacagcggctgagcttggaactttttcagagatgtttcctttgcttttagttcacagaaggctccttattggatgggctgtactggaagatgcagcccagcaaccagactgaagctgacaaccgaagtttcatcttctatgagaacctgctgttaggggttccacgaatac

SEQ ID NO: 241 - DNA, 32598 bp, Homo sapiens
  source 1..32598: mol_type = genomic DNA
aatggtagtagccactggggaatcattgcaacttatagtggagctggctattatctggatttgtcaagaacaagagaggaaacagctgcacaagttgctagcctcaagaaaaatgtctggctggaccgaggaaccagggcaacttttattgacttctcagtgtacaacgccaacattaacctgttctgtgtggtcaggtgtgtactgaggacatgcatccctcctatttctgtgtggttgtacatacatcctattctggggttagccagaaaaacctttgcctgcagttagctacatgaggatgccaaggacccagacggatagcaagggaggggtaaaaactgaaggcttaccgaaataaaggatatttgaggaagggagttgggatcctagaatattacgagttggaaagaaccataactctggtccaagttcatctcaatgctggaacctttccagaaaaagtattgtgtttttctaacatctgtctttacccattataaggatggttagtgccacatgttccatcaccaagtcccccggccatcaaatcttgactcatttcctggagtttctcactctcagatgagcctctgctattagcacacaagcacagtaaccggagtgcttgtaggatgctcagtaggatacccaggttacctgctcgtgctcagggctaccaaaggcacgtaaagttccttccacagatcctgggatgttgccatgatgacccctctgtgagatagtaacaaaaatgacaaagattccactggcttgtctgggactcttcttcattcatttattcagcaaacattcattggacacttaatatgggctaggcattgttctcggctcttgggacatgtcagcaaacaaaataaagatccgcaccttggcagagcttgcatccaaccaggaggagactagagaataaacattaaacaatacaaataaatagtatagtatattagaaggtaataggtactattaaaaagaaagaaaaagcagagcaagaggaagtcagagttcacccactttaatcttcctggtgagcatgtcagcaacacccaaacatcactaacatggattattgcatgtatatttacacataagataagaagtgtttattctcataatagtctttgtcatcattcttgaggttaagttcaattctgctttatgtggcttgttggattgtcccagtccttgtatttaacaacatttgcagaaaatagtaccacattaaatcaattatagattatcccttatccaaaatgcttaagaccagaatgtttgagatttaagaaatttttcaggttttggaatgtctgcatatatataatgagatatcttggggatgggacccaagtctaaacacaaaattcatttatgtttcataaatgaaacttaatgcacatagcctgaaggaaattttatttttcccttagggatgttgaatcaactgttgtgtgccagcatgttgacaatgacctgtcacatgaagtcgggtgtggaattttccacttgtgcgttcatggcggtgctcagaaagtttgggattttgaagcattttatatttcagattttcacattagaaatactcaagctgtccttgctcacagtggccaaaaaaaaagaaagaaagaaaagaaatactcaaccagtagtccagtagtagttatcactagaaatgaatgaaaatctattgcagtattattgagtttttcctaattattccagtgcagataaaaagaaaagaataaaaaggaagagaataaaaacagagaggcaactctgatattttagtaaattctatttatagaaggtcttgagtatttcttctgcttcctcccttactttaaggatgaacattgttaagacaactgcttcatttctctatactgtttttctaagtttctggaagtggttgactactgcagggccagaatgggccagagaaatgacttgacacttgaaggccacttccttcccttttgagttcccaatgaagctgtcacatacaaggctcttggcttcagagttgctctcctgagtttttgattctcacccctactctctaacacatcaaataggaaagaaagaacaggagaactgacaatgaaaaggaaggaaaattttcacacttctctgaccagttctaatttaccatagtcctgtttttacttgattattgctcatgcatgtgtcctgtatgctcaggttccaggtgcggctacctgtcctgtaatggcagagatagtgatggctagtagctgactaaagggcttttaaatgtctcaaaatgaagcagctagagattctatttctagttagaaaagaagtctgtatcattaactgaatcacccagctttctcagtgtgacacctacaaaatgggcatttgacaagaaaaaaaccctcagtccagttatggtaaagcagtaaagatcagagccatcagtatggatgtaaaatagtgtatgttttagacaatcagacatctattgagtacccacctattaactataaggctctgggaagaagagagaaaactaccctggaggacaaactatttgatgctatttaggtgttacataatgaatgaatgactcagttcctatctttatatatgtacaaaatatatcctacttctcaaccagattacacatgttttgagtggatggtttatatttctttatattcttcatgttgcctagtagaaggacttgaatttaatagaagtcctagggccaggcatggtggctccttcctgtaattccagcactctgaaaggcccaggcaggaggatcatttgagcccaggagtttgagaccaacctgggcaaaagggcaagactcagtctctgccaaaaaaaaaaaaattagttgggcatggtgctgcacacttacattcccagctactcaggaggctaaggcaggagaatcccttgagccctggaatttgaggcagcagtgagctatgattgcaacactgcactccagcctgggcaacaaagcgagtccctgtctcttaaaaaaataataacagaagtcctagaaaagtttgtgtgttgatttacttttacattaaaagtatatggcatgttgagcagcgtaaatatagaaaagtgtagggaagactgagcaggaagtactcctttgggactgaaagacctcaggaagtcttattcctttgatggcacaaaattctccaagtatggaattattagctatgataaaaatgttttgccgctagtttggggggactcatggtagcagtttcattaccttgtaatgcatgaacagaacagatggacatccattcctggctgtattcatgtgttgttgttgttattgttttaattgttcttatttacatgcaggttattggttgaattcccagcaacaggtggtgtgattccatcttggcaatttcagcctttaaagctgatccgatatgtcacaacttttgatttcttcctggcagcctgtgagattatcttttgtttctttatcttttactatgtggtggaagagatattggaaattcgcattcacaaactacactatttcaggagtttctggaattgtctggatgttgtgatcgttgtggtaggtttgagaacaacaccaaatttcctattctattctacaagcatgttaactagagtctttgatctcctcagcattgtggatcttgatattcccaaaaaagaatctaaaagtccccctcaattatatcaacttctgttactaattattttctcattttgcatgagtaactttgctgagtatgaagtggagaggtatttacagtatgctctcagccacgctaataacaagagtatctcagtaattcatatttggctttagtatgccgtatgagatgtggaggagaaaacagttttttttctttgtttttttttccactaatgatatttttcttcaactgctggtaaaaatcaatttatattttcctgcacatgtgtgaagttacagcaataaaaaaacttgtcggccaggtgtggtagctcatgcctgtaatcccagcacttggggaggccaaggcaggaggatcacttaagccccagagttcaagcccagccagggtaacatagtgagaccctgtctttacaaaaaaaaaaaaatttaattagtcaggcatggtggcacacacctgtagtctcagctattccagaggccgaagtgggaggatcatttgagcccaggaggctgaggctgcagtgagctataaatgcaccactgcactgcagcctgggtgacagagtgagaccttgcctcaaaaagaaaagaaaaaagaaaaatcatcctgaaaatattttgtgcggcagagaaaactttctgcagtttaaaattttctgcaaatagtctgcagagtacaaatgtaagttatattcatcaaagttttctgtatgaggtataagaaatcaaaggcaggccatgcacagtggctcatacctataatccctgcactttagggaactgaggtgggaggatcacttgagatcaggagtttgataccagcctgggcaacatagtgagaccccatctctaaaaaataaaaataaaaaataaatcaaaggcagagtcataatcaagaccatgacaccatgtaaattctgtgtctgctcttgactctattataacttctaagatttttttcaagatgttttcccttcatccttatcacttaattaagcatccgtcacttccttcctgtggtttcagtgtataaaagaatttttacaagcttttctcccttcagcaataacaggtaacatttcgctaagtccagttgtacatttaagcatataacaacatgcttaattattagatgcttacaagctttgcttggcataggtgtaccatgtattattctatgtctttccttcccactgtcctatgatagccattaccttctgaaatctcagtaaatgatgcactaccctattagcattctctcttctgttagccctccttatgagagttattctttccctcatcccactcctaaaaattcatttggcctttgtggagtatttagatcaagtcattattaaactattccccactagaattattaatagttgataaaatatggaaaatatattattcatatgtgagtgaaagaacacttaaagcataaaaaagaactacatggccatatattgcatggcaataactatattagcagcaataataattgtaatagtaataataataatggctaacactttagaagcttctgtgttaggcatttccgggtgcttttcatggtacagaagctcatttgttcctcatactaaccctcttcactactctgccactgcctctcaagccatggatatgtgcactttagagtttttacttaaagtaaaaattcctgtagaatagatggtgttgaccccatcagaccgtgatgcagttagaagtgcatcccgtcttttacgatggccatacaacatcacacaaatcaaggtaaacccttgatccccaaattctcaatgtattaataatgaacagagttacacaagaattttagcatttaaggaaacaggagagacgataatactggaaataatttttcagaatatttctgttcggattgatggcagagtgcaggccatatacattgacaattattccagaacacaattattgtttggagctaaaaggatgcaaaccctgcctcttggcttatatggatttatttatgtttaggccattgaaataatggtagaaaggtaagtatgatatgctaattaagaacagacttcctttttatattttaaccaaagaactcaatatcaacaaaagactagtcagtggtattcaccctttttgatcatacacacctatcagtgaaagctttgactactacacaccctaatttattatctttatttgtaaattgtgtgcctactttggtgtaacaggtacatccgtaaaacatacacatacatagaattttaaagattggaataaaagttaatacaaatagaattccaaaactttcttaccacctacctagaagttgtaacattttcttcccatagcccagtgaattgccttgcacctgctttggcgaccaataggaagaaaggcaatgatatcatggaaattgtctctttggctcaacagccacagcattttgcaagtgtttctttgaaaacttgcttcactgtgtttttcaattttttttttaaacaacacttaatactgccagacaacaattcagacagtgtgctttttgtttataaagaacaaggaagggctactggaatctcacttgtcctttgaaacttttgctgaccaagtgtagaagtgagggcatgccttctgctcacccacaaaacacaccctgtgctccactggaccttcaaaccagtggaaagacccaacgctttttgttttatctagccaaaatttgcttatgcttacccaaaacctgaaaagaaattatattcttaatataaatacaatcacatcttgaaatcactttgaaattttctttattttccttttttctttccccaacatatgttctgaagtacacaggctgcatcagtcagccatttgtcctgagcaatagtctttcaaaactagaagaattacttgaaaaaaagaagactattaaggaatttaaactcaaataatttattgaccacttgctaggttgtacagggtaaatttttttttttttttttttttttgagatggagtcttgctctgtcacccagactggagtacagtggcaccatctcggctcactgcaagctctgcctcccgggttcacgccattctccctcctcagcctcctgagtagctgggactacaggtgcctgccaccatgcccagctaattttttgtatttttagtagagacagggtttcaccgagttagccaggatggtctcgatctcctgacctcgtgatccgcccacctcagcctcccaaagtgctgggattacaggcgtgaaccaccgcgcctggcccgactaattcatttattcagcgagtgttttctgagcacctactatgtacttgccactgttctaggcactggggatacaggaatgaacaaaatcacccaaatctctgccctcccaattttctggtatggagagaaagttcttttgtagatagggatgagaatcccacagaaaactcaggagtgatgtaacaatgcaaatgctcacaaaatctctttcctcatctttcattccctatttggaaggaaaggtttcaaagacattgatgttgtataaatgaccatcttcttcattattttataaacatttgtcctgtgcagagtaaaaacaactggactgcataacaaattatacctattgagagttgggtaaagagttaccattggatccagtccaccaacccaaatgtttttctacatgtattagctgagatgagctcctcatctcaggagaagcctattcccactgcaggccttcttgagtctgctgtgttcatcattcccaccaccaacacaaaaatacaagattggctctggaaatccttcacagagaaggaaggaaaggaagatggtgaggttggctttttagctgtgatcagcaaccaagctggtctttgctatgagaatcagtgggaccatgatctctatggtcatctcaggaagggagggctaatgaagtggtctctggccatgattcctaagaagagagaatggacagcaaagatcgcagcacctgccacagcccacctgctgcagattcagaccccctggtggggccaggttgttatgactaacccctagcactgtctacatttagtggtgatggatgccaaggagggggcagtgtccccttggatctgattgtaaagcttagaaccaaagcatatgtggaaagttgtagggtcatgagttaagggacagaaatgagcaagagagaagcccttggctcatatattcccactgcaggccttcttgagccctctgtattcatcattcccactgtctttcaagcccaagctcatgctttgttctcatgggcactcatttttaggatatcttttgtccctcttgactttatgttgtgtgataccaactctttagtagttttttgtattatttaacttcatgtgtttatctgctcttgagtctcgagggcaggatttatgcagtttacttctgtttattcctcttagcccttgcacagtgctgtatacatagacatctactatatttttgttaaacagagcaaagaatggctggcaattggagaatgcagagaaaccgaaaaattttaaatttaaaaatcacaaataaaaacagcaggatgaaggcaagaaagcaaaagggtggaaagtgattaaaatgaaggtggcagaaaaaacagaaagcattcctctttgagtttgagtctgttatagtgtgatctcttctgtgtatgcatgtatgtgtgtgtaatgtatatgtgcacacacatgcatgcatgccttcgttgagtttctattccgaactaaggaaatgcaagcaatatactgttttacttattttatggcagggcttaacactttccatttgagtgagtgacttttaagaatgacatcgggtaagtataatggtgagcccttataattaatacattggtgaagaaaaatatactagtcatattaaggtaagtttcatatttctaaaacactgtaataaaatataaatattttgcttttcagctgtcagtggtagctataggaattaacatatacagaacatcaaatgtggaggtgctactacagtttctggaagatcaaaatactttccccaactttgagcatctggcatattggcagatacagttcaacaatatagctgctgtcacagtattttttgtctggattaaggtaatttataaatttcatgttctacattttaaataatattttctttaaaaaaaatgagttccacaaaatcatggaatacttgaatttgaaattcaagtgaccagccaaagctgctcaatatttactttgagacagggtctcactctgtcacccaggctggagtgcagtggtatgattacagctcattgcagcctcgacttcccaggcccaagcgatcctcccaccttatcctccgaagtcactgggactacaggcatgtgccaccatacccggctaatttttaaattttttcgtagagacaaggtctcattatgttgcccaggctggttttgaactcctgggttcaagcaatcctcccacctcagcctcccaaagtgctgggattacaggcatgagccaccgtgccaggcctcatattttacatataaagtaaactattgagactcatgtgatcattcctctcactgtcaatgacatacttctgctatctgaattagtgcaagatcagtccctataggttttgtttaacaaatgcagtaagaggcctttcagtgtgttagctgggcctggggccccaggctgctaacagatgagatgaacaggtgaaggaaaaggaacttagagaaagagagggaaggagcaggtggagggaaggggagagttgctgcacttggaaatgcttgctagaagggatcgcctcttttccaggtagaggctgtaagggaagctttacctagaattaaggttggaacagacactgcttccaaatagttccttgctcactattttccttattgtcccaagatataatgtgcatttccatgtgtgtgaaaggttatgacatttcatatacaacaagcctcaattctggagatgcaggaaatttcaataattctcaggcagcagctgccattcggtcaccagcacaggctctgattgtgctgtccagacagtaagtactagccacatgtgcctatttaaattcaaatttaaattagttaagcttaaatacaattaaaaacgcagttccttggtcctactggccacacattaagtgttcaatggctactgtctaggacagtggaaatgtagaacatttccatcatcacagaacgttctcttgaaaagcactgttctggaaggtacttacccgttatgtacttttctgagttggtattcatacctagaagacctgaggtttatcacaagacatagacttggaccaggcgcagtggctcatgcctgtaattccagcattttgggaggccgagataggtcccctgagcccaggagtctgataccagcctgggcaacatggcaaaacctcatctctactaaaaatacaaaaattagctgggggtggtggcacgtgcctgtagtcctagctacttaggaggcttaggcgggaggattgcttgaatccagaaggcggagggtgcagtgagccaagatcgcaccgctgcactccagcctgggcaacagagtgagaccctgtctcaaaaaaaaaaaaaaaatgcatagactttatcctgtatttctcatgctatttatttattgacatgcttgttcaagagaaaccatcactaaagcacaaaaccttgatcataacatagtaataataatcaaacagcaaaaataataatagtaataagaatgttctgtggtgatggaaatgttctatattttcattgtcctagacagtagccactaaccatgtataggcatggaacacttaacatgtggctagtaggaccaagggactgaatttttaattgtatttaatcttacttaatttaaatttgaatttagatatccacacatgtttggatagcacagtcagagcctgtgctggtgaaaggatggcaggtgctgcctgagaattactgaagtttccttgattattattagtttaataataataatcaagatagtaataataatcaagatagtaataataatcaagatctcagctgggcacagtggctcacgcactttgggaggctgaggcgggcagatcacctgacgtcgggtttgagaccagcctggccaacatggtgaaaccctgtccctactaaaaatacaaaaaaaaaaaattagctgggtgtggtggcacgtgcctatgatcccagctacttgggaatctgaggcaggagaattgcttgaacccaggaagcagaggttgcggtgagctgaatcatgccactgcactccagcctgggcaacagagcagcacttcgtctcaaaaaaaaaaaaaaaagatctcaaatgaattgggattgtattaagtaatgattaagtaatgtgattacagcaatcctcaagaaatatttcactgtggccagtaacaatgtgtaacagacctttaaacttctagagattttcctacaacatgtgtctcaggctgatgtgttttatttagtgcttctcttggaaatgtcttgcccctcgatactttatcattaaggtctttaaggcagggatcatgactctacttttttttttttttttttttttgggacggagtcttgctctgtcgcccaggctggagtgcagtggcacaatcttagctcactgcaacctccgtctcctgggttcacgccattctcctgcctcagcctcccgagtagctgggactacaggcggctgccaccacgcccggctaattttttatatttttagtagagacggggtttcaccgtgttagccaggatggtctcgatctcctgacctcgtgatccacccacctcggcctcccaaagtgctgggattacaggcttgagccaccacgcccggcctcatgactctacttctaatatctcatcatgtgctcttccactgaggcttctacttagagctacacaatctgggcagccatcctcagtgccttatctaccaacatgctcaatatggctttgcagggttcactgtctaccagcagggttcactatctaccaacatgctcaatatttctttgcagtcaggcagagcaggctttgcagttcaggcagggcagctggctgcaggccccagctgactcctggggatagaatgccaatatttcagacattgcagagatttgaggcaatgtacataaagccctccacatataactgatgcacaataaatgacagttaatattatgcaacaagaatttcctggggggttttataattaatttttatttgtgtgaagttttttccctcccttttactttaatcctttttggggggaaagcatcactagtcacagttcacggcagcctcgacctcccaggctcaagcaaccctcccacctcagcctcctgagtagctgaaaccacaggtgtgtgccaccacacctgactaatttatttttattttctaatgaaacagaatcttgccatattgcccaggctgatcttaaactcatgggctcaagcgatcctcctgcctcagtcttccaaagtgccgggattatagatgtgagccactgcactcagcctttttttttttttttttaattgtagatagcataaaacctactgttttaaccatgcttaagtgtacaattcagtggcattaagtacattcacagtgttgtgcagccatcgccattatgctgcattattttcagaactttttcattattctaaactgaaactttgtatccattgaacactaactcccaattcccccagtccctggtaacctccattctactttctgtcactgtgagtttgactattctaagtacctaatttaagtggaatcatacagtatttgtccttttgtgtcaggcttatttcactttgcatgatgttttcaaggttcatccatgttgtaacctgtcagaatttaatttcttttcaggatgaaataatgttttattatatacagtcacaccattttgtttatccattcatctattgatgtcttctctctcttacagctcttcaaattcatcaattttaacaggaccatgagccagctctcgacaaccatgtctcgatgtgccaaagacctgtttggctttgctattatgttcttcattattttcctagcgtatgctcagttggcataccttgtctttggcactcaggtcgatgacttcagtactttccaagagtgtatgtaagtatatatgaaattaagaagaaaaatttaatcagagttgtcactgcttctcaagaataaatcttcatatgaggttgctatatgaccaccaattatttaaaaccagttattttaagtaagaattaattaccttttcccaaaacattgatctacccatgcaaagaagacaatgcatcctgaaatgctgatgcttaagatagcagcccaaagtagtaaaatacagttaacagacataggaaaccaacactgttctgaagactgagtttttctttgcaccaaatgcagatggtagcttctagaaggctgtttgcctatattcttactcctgttgaatattgttgccatatatttagaacttcaagttattttctaaggaaaaaaacaagatatttctaatattctaaggtaaactcagaccagtacaagaattttcagtttttttttccaaagatcccaaatgtgaaataaaacaacaaaaagcagccagtgtcagatttctatgccatttagaaaggagttagtttaaaaaggaatggaagtaatagggttttgtgcatagatatctcgaattaatattgctgttgataaaagtgattttgctaagacccagcactgacaacacttggccactttgatcccattttaagtacttgtcagaatattggatctttgaactcaaaccattttgggtttttggggtttttttgttttgtttttttttgttttgttttgtttttgaggcacggtcttgctctgttgcccaggctggagtgcagtggtgcaatcatagctcactgcagccttgaactcctaggctcaagcaatcctgctgcctcagcctgctgagtagctgggactacaagtgtatgccaccatgcctggccaatttttaactttttttatgagaagggatctcactgtgtagcccagggtggtattgaactccagggcctcacactgtcctctcacctcagcttccaaaagtactgggattacaggcatgagccaccacaccaggccctgttgttttttttttaaagaaatttttaactttagaccgagggtgactgttgtcaaggtttagggttaagatgttttacctagattatgtgttgaaatgttatagccaattgctttataagttattgaataataattgtattttctttttttttttttttttgagatggagtctcgttccatcgcccaagctagagtgcagcggtgtaatctcagctcactgcaacctctgcctcccgggttcaagcgattctcctgcctcagcctcccgaatagctgagattatgggcgcacgccaccaagcccagctaatttttgtatttttagtagagacggggtttcactatattggccaggctgttctcgaactcctgacctcgtgatccgcccgcctcggcctcccaaagtgctgggattacagacgtgatccaccgtgcccagcttgtgttttctttttaaccaaatggaaataacctctgtagcatgaaagcattttattattattgcagaaggctttaattgctgatacaagtagcaagactttgtaaatgggattgacaattttctgttattcggcagctacctatactgctaaaaggtccaaaaataatgaaatcatctttaagaaatgttgcatcaactagtggacattctttgtttttgtattgtggtgttttgttttatttttatagcttcactcaattccgtatcattttgggcgatatcaactttgcagagattgaggaagctaatcgagttttgggaccaatttatttcactacatttgtgttctttatgttcttcattcttttggtatgtacatttttatttatagtgaggttcaatttaaacttcgtaaatccttgtcttctcttttctctcacactttatgtcctatcaattttaaataaagacccaggaagtagaaaaaagtgtggatgttggaaaacttattttccttttattaattcacagttttgagactcatatcaaatgtcttttctgtggtctttcattgatccatgtatatgtgtctattcaatgcaaaaaaaattagatctcttccatggtctttcatttctctctctatatatgtatctattccatgcaaaaaagaaattagatcaagtacaaatttataaagatacctaaaatagtgctttgcctaaaaagtagaatatgcttacatgctttttaaactcatatgtcagcactttcgtagtcacttgctagcatgacttttctctctttcttcttttctttttaaaaaataagaacggaaaagcaagctagatctaagatgtcgagtaatagttgagtgaatcattgcatgtcaaaattaggatattctgttttaaattatttatatcccattcatctagagactgcctacagagaatattcaaataattaagtttaaaactaaatgtaacaatgaatggaaattgcattaaaattattttcaaaaataatttttttattctcttgatttggtacaaatgaacatttttaatgtttttgccctaagtcaattaagtttttttaaggtgttttgttctttttcttaacatttatatattcaattgtctactgagaaggtgttaagccagcttaatttaggcaatatttttcatctaaacactaacagtcatcttaagaacaattttcttaagaaaataacattttttccatttcagtaaattgtgtaaagatcccttgaggaaggttaagtgatcacattttcagtaattcagtgtaataactctaaagtcagtccaggtattactggttaagtatatggtatttattgattgggtattagatgtactgtattaatttcctgtttaaaaaaaattttttttccggggagacacagcctctggtgtaaaacaaaggtgtgttccctagctgtactttaacaggactgaaaaggtcaggaatatcattcaagttcatatgtatcttgctgtatgcatggtttatggctcatttttaaacttacacctcttaagcttcttcttcctatcatatattaaaacaatggagagaagaataagcctctgttactctaccattgatagtacttcggattctagagtacctgaatctctactaagaaggcaaaaaccaggaattgagagtcctgcacctgacccttcagttgatctcaggccacctagttttctccgtttatcaatctgccaaacaaggatggatagagtcgtggcaactggaaaggctcaaatgtggaattgtttgaatgtggtcctttagtaggcagccatcttaccagatctagagtattcagtcatcttaccagatcagtcaccagaacatgaaaagaagctcttagtttctatctttatactaaaattgtttttttgtacgactgcacaaaaaagaattgctctccttgcacctcccagagatataggtggatagatacatacatacgtacatacatacatacatacatacatacatacatacatagacacatacatagatagaagtctactttcaatacaaacctgtcttttaaggaaatgacaagctgagcatagggttggccacctttctgagccgattgcctggtattagtttattgcccctgtttagcaagaaggcacagtgttaagaagtggctcagctgaaccaggataaccccactcttcccccacatcaacaggaaagacatcctggtgcagatgtccatctgataattcagggaacctcgggagacaggatggagaggagggtgagctagcttcctcttcccacaccttcaagagcctttctcaagcactttctattttttgaaatctctttagaggtcccagactttgatctgtttcaattaaggtattggcaggcattagttaacagccacttggaagcaaaaatagaacattagatccctgagttggaagagagaaggtagaaggtgttacttggactgcaattatctgcacttggaattgagcatttagtcaaaaacttatatgtattctatattctattctcatttctgctacagaattgtaaacaatattcttccttaatacagaaattcatagcccactaaaataagagcgttctcatttgttcatttctcaatcatttaataagtatttactaagccactatatccatatatatatatatatcatatatactgtataatacacactgtagtgttttttgtggattgtgtactatgaggtagtatgttagatactgccagtactggggtaaggaaaacagcctgattaggcccttacgaagattcctcagacttgtggggaaaacagacattatcaaatagaaatacttgcaaaccacagttatgtgttaaaaaggaaaaacaaagtaaaaaaaaagttggtgggggggaacctgatctcctggatacagtgcttcgagaaagtttgttgttggaaatgcaaaccattactactgtggaagggaaaggtcagaaaaatgaactcaccattactgaatagtaatagtagctatcaattaggtggcacttacctgcatcaggacctgtcctgagcactttacatagattgtctcactaaccagcccaacaaatatgtaagggagatactattatttttcccattttattaatgtaaaacaattaaataattctttaaaattagacttagaaaagtggagcaacaatcttagcagtgctaggactgaaatccaagtttgcttgactccaaagtctatctctcttccagaaactttttctttactatctgcctagtaggcctgctgtattcctatttgcaacagccttttaaactctttaaaaatgtgtcctgtaaatttcatatatgattatacaaaaaaacttggaataagcatacaattctacttatctgtgttaactgttgaaatttgaagagctttttggaattctatacccttcagtagtgtatgtaaaagtttctaaatatagagaacatagataagcaaaaataatattaaataaaataatcgcaccattagtaggtaaatatactaatattttgttgtattttattcttgtatgttttcacaaagtatatcataaaatttttcctgtggcatgacttaacggagaaaataatcttcccaaaacatgtggcagcaaaactgttaatttattacatcaggctgggcacagtggctcacgcttgcaatcccagcactttgggaagccgaggcgggcagatcacttgaggccaggagttcgagaccagcctggccaacgtggtgaaacactgtctctactaaaaatacaacagttagccaggtgtagtggcacatgcctgtaatcccagctactcaggaggctgagactcaagaattgcttgaacccaggaggcagaggttgcagtaagctgaggtcgcgcccctgcactccagcctgggcaacacagtgagactctgtctcaaaaaaaaaaatttttttttaaataaataaataataaatttatgtcttcataaagcactcagattaggaaaaaaaggataaacaaaaaggcatgtgtcatttttttgattgataattccaaattatgtttcttcctttaatttttgccctcctttcatttacaaacagaatatgtttttggctatcatcaatgatacttactctgaagtgaaatctgacttggcacagcagaaagctgaaatggaactctcagatcttatcagaaaggtaggaaaaaccttaattctcagaattcttctgtttctgacataaaatgagcattgtttcacccagattttcaaatcaacattgatccattgaaattgtttgaaataaagaatacattgctatatttcaggaataatttaaatgttccctatcttggagtcttgatggatatactgctatcttgaattttaattctgggaatccttttatgccctggaattaaattctcaacaatcttttgacactttaagagctgagctgaaggttcatcaccttcattattttgacatctcctgtagctggctctcacttcaggatcctgagttgagaataaactagaagggaagattatataaagggatttccacctcttctgtctcaattaccattttaaaaaaataaaaagttttagaggaaaacacttagtagttcaccctttacccttgaccttccacggcagttttaaaataagcaaaggaaaagattcatgaattcaggccatagcctggggcctgagaacttttacttatgcaccttctcaggaagggtttcattgttaaatagaagggcaggacaggaaagttgggcctctttgttcttctcaatgtaacttctttatttggtttaaagtataaaatgtatacaacaacaaataaccacatttaaaatacacagtttgtttcccaacatcattttgctaagtcatagtggctccttaactgtaatttttttttttattagtccaagccttaggattatgttatctgtgatatatgttataatagaaaacttaagcctcttaaaacaaagtccttgggatgggacctaagattcacattatcttgattccgcataacagttgcttacattttagcaaatctccagtgtgtatgcaagcactcctcacttggcacaattctgatacacacaaactttggttaccccagttttgttatgtaacaccaccttcaacaacacagttcaaatttcagttatcatagtatattaactctgagtaacagcacaaagtacaaactccactgctagctcttcagtgtatagatcagttacctgagtaacagatgtgcaggctgagcaggctcactggtcagtcatgacactattttcagtctgttactaattggtcactgagcatctgctatccaattcacaaacaaagaaagcatgtagtgttgcttccttgtgttccagtaataagcccatgtgacattttacaaaaatggataattgaaaaagagaatgggtgcagtggctcacgcatgttgggaggccaaggcggatggatcacctgaggtcaggagttcgaaaccagcctggccaacatggtgaaaccccatctctactaaaaatacaaaattagctgagtgtggcgacagatgcctgtaatcccagctactggggaggctgaggcaggagaatcgcttgcacctaggaggcggaggttgcagtgagccgagatcgtgccattgcactccagcctgggtaacaagagcgaaactccgtctcaaaaaaaaaaaagaaaagaaaagaaaagaaaaagagaatgggctagcaaagaaatgaaaaatggtaacactggaagtgaaaatcaaaacagagtaatggatttatagaagaaatagctgagtgaagaagaaataggagtgttgacactgcgatcattcaagagatccagatatggagccagaagaacttagggcaggtctatcaacttaaatgaggaaaatagctgtgataaaacagatgaagatgtcttgaggaaatgatgcctgcaaaaaacttcacattaagggaagtcttattagagatatttcacaataatgaaagtacaaaagaaaaaatgttggggctggacatggtggcttactcctgtaatcccagcactttgagaggccaaggtgggtggatcacttgaggccaggaagtcgagacaagcctgatcaacatgatgaaaccccgtctctattaaaaatacaaaaattagccagacatgatggtgcacacctgtaattccagctactcaagtggctgaggcacgagaattgcttgaaccagggaggcggaggttgcagtaagctgagattgcaccactgcaatccagccaggtgacagggtaagactgtgtctcaaaaataaataaaagaaaaatatgttggaagctcatccacatttaagaaggaatatgacaattcactaatgcatagaaaagaagttcactccacattgtaaagtgtacagtgtaatattatacaatgaaaacaaggcaagtgctgtttaaactactctggatacattttttacaaagaaataaaacactttagtttttaatgtttctaatgttttacattttagtgtattaaatcaatattagttttcttcttttttaagctccctatacatttataactgacactaagggagtgtttaatgttttgattaaaagttgtaaagatcacagaacaattgtaattcttcccactgattattcagatcattttgcacaatttcagcttgcatggtcacttacagtgccgcactatgtgcaaagcaaggtcaggtctaaagttcgctaatgaaaaatcctcggccaggggcagtggctcacccctgtaatcctagcactttggaaggcgaggcaggcagatcgcttgagctcaggagttcaacaccagcctgggcaacatggtgagaccctgtctctacaaaaaaaaaaaaatagcaaggcgtggtgactcacacctgtagtcccagctacttgttgggggctgagttgggaaaatcacttgagctcaggaggtcgaggctgcagtgagccagaatcacgccactgcccttctgcctgggtgacagagtaagatcctgtctcaaaaaaagggaaaatcctcatctacatttcactgggttttttgtttgtttgtttgtttgtttatacacacttaaggaaattactgtctagaagatagataatataaaaaataaaaatgcaattcatgattcgggtttcttggtattcctaagaactgttgcacagtactttatgctctgaggcagacagctatagcatatatagtaatttttgtttctatcacataaacttgaatacacatatgagtaaaagacctttagttcttcatgacttactgaaagaccctgactttttccatgtaactgttccacaagtgttttatggaaaactggatacattaattcttcattcatccagcacgtacttgttgaatggccaatgtacaccaggtttgtagtagttactactgtgaatggaaagtaaaacagatgcaaaaggagaatacactaaaccaagtcttttattttttctctctctgatagggctaccataaagctttggtcaaactaaaactgaaaaaaaataccgtggatgacatttcagagagtctgcggcaaggaggaggcaagttaaactttgacgaacttcgacaagatctcaaagggtgagaatcatgcttcctgaggttctgaaaaattcctgcttctaaagataaattcctggtgataagagtatttctagcccaagggctcatacagatactttttttttttttttccagaggcaggtatctttctggaacatgttataagaggaaaacttgcccccatttggtgatttctcctttcctcctgcattttgatgtctctgtgttgagggtgaactgggtacaaggaatgatttttatctgtatcctctctctaatttcaggaagggccatactgatgcagagattgaggcaatattcacaaagtacgaccaagatggagaccaagaactgaccgaacatgaacatcagcagatgagagacgacttggagaaagagagggtgggtctggtttaggagaaccggatttgatttggtacctacaacaccacagatgtatcaaacactatagaagtagtgggttattgagtctcttgcccattccccaccacactctctctctctctcagtcggtttatgtgttagtaccctgtttattccagaaagaatatataacacaattatgtataaaaatgggtggttagcatgatataaaaacgtcaaaatgaaaagcaagcaaaacaaaagtaaaaataatggattattaatgaagcttaaaaatgcattcataaaaacacatatgcttattaagattgggctacaaattgggccctaagcttgctggtaatcagcttgaaaagagaagcctgattagctgcagagtccacaatgtccgtgagagtgaagaaaacaaaaaatgacttaccaagagatgtgaaattattctggttagttagtggctatttaaattgttaacttttttttcttttttttttttttttgagatggagtcttgctctgcctcccaggctggagtgcagtggcacaatcgcgactcactgcaacctccacctcccgggttcaagcgattctcttgcctcagcctcccaagtagctaggactacaggcacatgccatcatgcccggctaatttttgtatttgtagtagatatggggtttcaccatgttggtctcaaactcctgactgcaagcaatctgcccaccttggcctcccaaagtggtgggattacaggcagtagccaccgtgcctttcctaaattattaacatttataataaaattaacagccgccttccatttgaatactttttacaaaatagttaaaaataaacataagtgggcttttatagtcagaaaaaaaaattcaaagctttaccattaactttcaaaaataaatggttagacagcaacaacaaaaatctgtggtaactgaggtacagagaacacagatgaatgttattacaaaagccactttcctatgagaagtctaggacagtggtttctaaatgccactccacagacagtgctagtaggtgacagacttctccagtcacagtgaaatttaagcataaagaaaatgaggaaaatttttacaaggctctatttagacaaagttcttattctgacattacatctttcctactttggagctgttgaatgtattatcttttatgaaaagaaggcgatccaggttgagcatccctaacccaaatatgtgagtctgaaatgctccaaaacctaaaacttcttgagcacaaacatgatagtcaaaggtcatgcttaaaggaaatgctgtcattggagcagtttggattttgggttttcagattagggatgctgaaccagtaagtataatgcaaacattccaaaatatttttgaaaatcccaaatccaaaacacttctgatcccaagtatttcaaataagggatactcaacctgtaatatatttcttcatttctttatttattttattattattttaagatggctcatggcccactgcagcctcaaactcctaggctcaagtgatcttccgacctcaacctcccaggtagctcaggtagctgggactgcaggcatgcatcaccatgcctggctaattttttaaaaaattttttgtggaggcagagtctcaccttgctgcccaggccagtctcaaactcctggcttcaagcagtactcctgcctcagcctcccaaagtattaggattacaggtgtgaccactatgcctggcccatatttcttcatttagttttttctttgcctgctgtgtttttaatgttctttcttgttcaaacaaaaagttggctattccttgctgttagttaaatttgccaatctatgaaactgaaaaatgcaggagtcccagcctggtgttaaatacaaagaaatcccaggtaaatggcatgcacccagttcctgcttgcccaagtccttggtgaggcttctgtggggtctcagtgttctgctcctcactcagtgaccccttgttcttcaggaggacctggatttggatcacagttctttaccacgtcccatgagcagccgaagtttccctcgaagcctggatgactctgaggaggatgacgatgaagatagcggacatagctccagaaggaggggaagcatttctagtggcgtttcttacgaagagtttcaagtgtaagtataaaggaattggcagaatttgcgttgacaagagtccacatgagaccaggcagttccctcatctctctgaattcactcctttccattactaatcatccagcttttaaaaataacttatactggccagacgcagtggctcatgcctgtaatcccaccactttgggaggccaaagcaggcaggtcatgaggtcagaagttcgagacgagcctggccaacatagtgaaaccccatctctattaaaaatacaaaaaattagctgggcatggtggtgggcacctgtaatcctagctacttgggaggctgaggcaagagaattgcttgaacccgggaggcggaggttacagagagccgagatggctccactgcacaccagcctgggcgacagtgcaagactctgtctcaaaaaaaaaaaaacttgtcaattggtgttttgtttcttacataatatgtttactataaaaattagcaaataagagcaaaagaaaacattaacatttcacatatttctaccaatgaaaaatgtttattaatatatcagtgtttgtgtctctatttgcatgtgtttatcaatgtttctatatatttttatggcctaacatatggttcgtcctgaagaatgttccttgtgcatttgagaagaatgaatattctgctgttcaagtgttctgtagatgtttgttaggtctagtttgtttacagttttattcaggtctcccatttcctggttgatcttagatgtgcctagatgtggtattcacgtttgaaagtggggtattaaagtctccaagtattattagttggagttcatcccttcaattctgtaaggttttgctttgtgtattttggaactctgttgttgggtgcatacatatttataactagtatattttcctaatatattgaccctattttctctctcaacttaatgaggctaaagaaaaaaaagaattgaccctgttttcattacaagatgttatccactttatctctagtaaaattctttgttttaagtattttttgtttgatattactgtaaccactccagctttcttttggttgctgtttgcatgataaatctttttccatccttttactttaaacttatttatatctttcagtctgaagtatgtctctcctgtagacagcatataattggatcttatctttttatccagtttgacaatttctgtttttgattagattgcttaatccattcatttaatgttatcattgatgtagttgatttctgtctgctattttattttttgttttctagcttacttttttttgttcctctttcactgctttcttgtacattaagtgaatattttcaagtataacatttaaatttttttaatgatttttcattttttttagtcaggagttgctctaagacttagtttatacaattaagttatgaaaaattacttcagatatatattaactgaatccagtgagatatagaaatcatttctatttagcttttttcctcttccctctttttgtgctatatattcatctatctatatgtatatatagtcatctacatatgttgcaaatcacattgttagaacaatgttacatttttataacacactgtgtaatatatagtatataattttatatctcttaatgaagctgagagaagaggaagtatatatttataaatgtgtatattaacctacttttttaccatttctaattctcttcttttgctcctctggattcaagttatcatctgtcgtcattctctttctccgatacagctttgctactgcctacctcctattattgtcaaatatattacatttctattatagaccttcagatccaattatgtacatatttttacacaactgctttttaaatcagttaaggaacaaaaggagaaatgtacatatatactatattttatacctacacagttatctttaccagtgttctttgccttttcatgtggattctgattactatctggagtcacttgctttcagcataaagaatttcctttagtattttttgtaaagcaggtttgctagcaatgaattctttcattttttgtttatctgagaatgtttttctttctccttcatttcctctggcttgtattgtttctgatgagaagacaggtgctaattttactgtggtccccttgtacatgatgactcaattttctctcaccactttcaagatttttttgcttttgtctttcattatttttactgtgatatgtctgggtataaatctctgagttcatcctacttagaaagtgttttttctgcttctttcactttctcttctcctttgggacccgcattatgcatatgcttaggggtatcacatatttctcttaggctctgttcgtcgtcattttttttccctctctgttcctcagagtgcatagtctgtattgatgtatcttcaagttcactgacttgttcttctgtcagcttgcttaaatctctgttgagctcctctagttatttatttatttatttatttattttatatatatatatatatatatatatatagagagagagagagagagagagagagaaagagagagagagagagacagggagacagggtctcactccatcacccaggctggagtacagtggtgtaatcatggctctctacagcctgaacacctgggctcaaatgatcctcctgcctcaacctcccaagtagctaggactatgggcacatgctgccatgcctggctaatttttaaaaaaaaatttgtagagatggcatcttgttatgttgcccaggttggtctcaaactcccggcctcaagtgatccttccgcctcggcctcccaaagtgctggggtcacaggtatgagccaccgcacttagcctgaattttttatttattatacttttcaactccagaatttctatttggttctttttagcaatctctgtctctttattctgtatttgatgatatcctgtatttgatgagacaatgtcatcataacttccttttttttttaagagatagggtctctctctgtcacccaggctagagtgcagtggcatgatcctagctcactgcagcctcgaactcctgaactcaagcaatcctcccacctcagcctcctaagtacctgacactacaggcatgagccactgtacccagctaatttttattttttgtagagatggggtctaagttgcccaggctggtctcaaactcctgggctcaagtgatcctccctgctcaggctcccaaagtgctgggattacaggcatgaaccactggacccagcctcctttatttctttaaccatggtggggttttttattatttgtttgtttgttttaacctgtttgaatatatttataataggtaccttcaggtctttgtctgctaagtctgacatctgggccctctcaaagacagtttcagttgtctttttttttttcttcttgtgtatgggaacattttcctgtttttttgttttgttttttgttgttttgttttgttgttgttgttgttgttaaagattggacatgttagataatatattgtaggaactctggctagtgattccctttcctccccaggacttgtttttatttctctttgcttgtttactttttcatacagtctgtttccccacagtgtctgcctctgactttattccttagagggcgcagctgtggccatgtacgtagtcactctgggaagacagtggttttagcagcgtgctcattaactttctctgacctctttgttatacctcctgcctctgtggatattagacccagttattacatttcattgtttactgattggtctattgttttccagaatgccttgggatgtaatttgctccacagtctgatccacttaaattcaggcctctttgcagggctagttttagaggccagtctttttttttttttttttttttttttgagacagagtctcactctgttgcccaggccggaatgtagtggtgcagtctcagctcactgcagcctcaatctctcaggctcaagcaatcttcccacctcagcttctcaagtagctaggactacaggtgcacgccaccatgcctggttaatttttgcattttttatagagatggggttttaccatgttgccccggctggtctcaaactcctgagttcaagcaatccacccacctcagcctcacaaaaggccgggattacagatatgcaccaccacgcctggcccctgaggccagtctttaactgtcttcttagctgtctctttccctggttctctctggtgaactagctggtaatttgtttatctcataaggctaccagattccttgtaaatgcttatccccacaatctccattgttttcaagagcatcgttagtctttaatttcctcacgcttcattccaaataaagtccattcacttgagaagagttctatattcctatgacctgtgtctccccatgagcaaaactgctactgctttacagagccagggacagtggcccacctctctgtggcatcctgctttatgaacaagtcactgggctcagatggcagtctctgattttctcaccttgcttcttctggcatggaaactccaccctacaagtgggaactgagtggaagaagagagccccattccccttagccactcttaacaggattagaacctctgcaacatgcatttaagaatgggaacaggctgggcacagtggctcacgcctgtaatcctagcactttgggaggtcaaggcaggaggattgcttgagccaggagttcaggaccagcctgtgcaacatggtgagaccctcatctctacaagaaatagaaaaattaactggtgggttgtgtatacctgtaatcccagctactcgggagcctgaagtgggaggattgcttgaacctggaggcagaggttgcagtgagtcaagattacaccattgcacgccagcctgggcaacagagtgagactctgtctcaaaaaaaaaaaaaaaaaaagagtaggaacattgaggctgggcatggtggctcatgcctgtaatcctagcactatgggaggccaaggcaggagtatcacttgacgctaggagttcaagaccaacctgggtaacatagcgagactttgtctctattaaaaaaaaaaaaaaaaaaggtgtaaaaaacttttgtaacaagagtgggaaagccgggcacagtggctcacacctgtaatcccagcactttggaaggccaaggcaggcaggcggatcacctgaggtcaggagtttgagaccagcctggccaacgtggtgaaaccccatctctactaaaaaatagaaaaattatctgggcatggtggtgcacacctgtagtcccagctactcgggaggctgaggcaggagaatcacttgaacctacgaggcagaagttgcagtaagccaagatcacgccactgcactccagcctgggcgacagagcaagactctgtctcaaaaaaaaaaaaagagtggaaatgttaggatgagaaatgctggcagcctgcccctccctgggagatactgtagccctagactggaagttgggggaggagggagccctgtgctcttagctgcacccatgtgaagttgtgcttctatcacatgagctggggacaggagagaaggctcagattatggcttcagtgccacagaatctcttcatactaaaatttagtagattttcttgaataaatgctttttcatttgctgtacacccttaaaagtttctagaaatttttaatatttgagttttaaaaaataattttcaacagttacagttatttcactaaagagagagtctacagaaccctcttgccaccattgcagaggttgtctttggcttacgagtttttaaagtatttgtatacattttttaagttcaaaataatagaattgtaagtgaacatgctgttttcatactgtttttcaagctttatttaatatattgtaaatctaattctattttattaaatagtctgccacagtataatgtctgatgtctccttagaattttattgtatggatgaacaatgattatttaatttcctaccaattgttgggtgttttttgtttgtttgtttgtttttgagactgggtctcactctgtcacccaggctggagtgcaggagtgcggtggaatgatcacggctcactgcagcctcaacatcccaaggctcaggtgatccttccacctcagcctcgcaagtagctgggagtacaggcacatgccaccatgcccacctattttttagagatgaagttttgccatcctgcccaggctggctcgaactcctggcctcaagcgatctgcacacttccgcctcccaaaatgccaggattacaggcgtgagccatcatgccctacccccccatcaattgtttgatgtagccatttttcaatgatccgcgattaagaagcagcactcttttatagccaaaaattacacatatataaaattttcctttagaaaatgttctaaaaatggaatgtctaactaaagggttaggcatacattcttaagacttctgatacgcgctgacttgcaggaaagttgtttcagttaacactcctaccagcggcatccgagagttaatctgtaaagcttgagacaacttagaaagtgtttcaaatgattgtgttgcttaagaaaaaaatcttagcacttccttttgaaaagccagtggggctgaaaagacaatgacaagcactttgtccctctgtactgtgttttccttgcagcctggtgagacgagtggaccggatggagcattccatcggcagcatagtgtccaagattgacgccgtgatcgtgaagctagagattatggagcgagccaaactgaagaggagggaggtgctgggaaggctgttggatggggtggccgaggtcagtagtcatgagctgaaaacaccgctgctgagcatggtgttattaatgaaaatatatgttgctgacagttgtatttgaagtattgaagaagagtaaaaaaaatttacgtttatagaaattcacaatgatgtttccatttactctcattttcagatttttttctctgaaacagaaacactctttctataaaatctcttgctataaaacatcaatgtagtcatattgtctaacccttaggctgagatgtttatctttctccataactacagataaaattataatctggaggtgttactttcttaatactccatatgctaatggtcctgccttcactgcagggtagaattaagtgaaaaattactccagcaactctgagatttgctattatatgctgtaaatctccagccttaccaaactacagattatttggtccctggacttcctaaggcatttccttctactgcccccaacaccagtttctttttccctttttaggatgaaaggctgggtcgtgacagtgaaatccatagggaacagatggaacggctagtacgtgaagagttggaacgctgggaatccgatgatgcagcttcccagatcagtcatggtttaggcacgccagtgggactaaatggtcaacctcgccccagaagctcccgcccatcttcctcccaatctacagaaggcatggaaggtgcaggtggaaatgggagttctaatgtccacgtatgatatgtgtgtttcagtatgtgtgtttctaataagtgaggaagtggctgtcctgaattgctgtaacaagcacactatttatatgccctgaccaccataggatgctagtctttgtgaccgattgctaatcttctgcactttaatttattttatataaactttaccca

SEQ ID NO: 242 - DNA, 23828 bp, Homo sapiens
  source 1..23828: mol_type = genomic DNA
atactttccccaactttgagcatctggcatattggcagatacagttcaacaatatagctgctgtcacagtattttttgtctggattaaggtaatttataaatttcatgttctacattttaaataatattttctttaaaaaaaatgagttccacaaaatcatggaatacttgaatttgaaattcaagtgaccagccaaagctgctcaatatttactttgagacagggtctcactctgtcacccaggctggagtgcagtggtatgattacagctcattgcagcctcgacttcccaggcccaagcgatcctcccaccttatcctccgaagtcactgggactacaggcatgtgccaccatacccggctaatttttaaattttttcgtagagacaaggtctcattatgttgcccaggctggttttgaactcctgggttcaagcaatcctcccacctcagcctcccaaagtgctgggattacaggcatgagccaccgtgccaggcctcatattttacatataaagtaaactattgagactcatgtgatcattcctctcactgtcaatgacatacttctgctatctgaattagtgcaagatcagtccctataggttttgtttaacaaatgcagtaagaggcctttcagtgtgttagctgggcctggggccccaggctgctaacagatgagatgaacaggtgaaggaaaaggaacttagagaaagagagggaaggagcaggtggagggaaggggagagttgctgcacttggaaatgcttgctagaagggatcgcctcttttccaggtagaggctgtaagggaagctttacctagaattaaggttggaacagacactgcttccaaatagttccttgctcactattttccttattgtcccaagatataatgtgcatttccatgtgtgtgaaaggttatgacatttcatatacaacaagcctcaattctggagatgcaggaaatttcaataattctcaggcagcagctgccattcggtcaccagcacaggctctgattgtgctgtccagacagtaagtactagccacatgtgcctatttaaattcaaatttaaattagttaagcttaaatacaattaaaaacgcagttccttggtcctactggccacacattaagtgttcaatggctactgtctaggacagtggaaatgtagaacatttccatcatcacagaacgttctcttgaaaagcactgttctggaaggtacttacccgttatgtacttttctgagttggtattcatacctagaagacctgaggtttatcacaagacatagacttggaccaggcgcagtggctcatgcctgtaattccagcattttgggaggccgagataggtcccctgagcccaggagtctgataccagcctgggcaacatggcaaaacctcatctctactaaaaatacaaaaattagctgggggtggtggcacgtgcctgtagtcctagctacttaggaggcttaggcgggaggattgcttgaatccagaaggcggagggtgcagtgagccaagatcgcaccgctgcactccagcctgggcaacagagtgagaccctgtctcaaaaaaaaaaaaaaaatgcatagactttatcctgtatttctcatgctatttatttattgacatgcttgttcaagagaaaccatcactaaagcacaaaaccttgatcataacatagtaataataatcaaacagcaaaaataataatagtaataagaatgttctgtggtgatggaaatgttctatattttcattgtcctagacagtagccactaaccatgtataggcatggaacacttaacatgtggctagtaggaccaagggactgaatttttaattgtatttaatcttacttaatttaaatttgaatttagatatccacacatgtttggatagcacagtcagagcctgtgctggtgaaaggatggcaggtgctgcctgagaattactgaagtttccttgattattattagtttaataataataatcaagatagtaataataatcaagatagtaataataatcaagatctcagctgggcacagtggctcacgcactttgggaggctgaggcgggcagatcacctgacgtcgggtttgagaccagcctggccaacatggtgaaaccctgtccctactaaaaatacaaaaaaaaaaaattagctgggtgtggtggcacgtgcctatgatcccagctacttgggaatctgaggcaggagaattgcttgaacccaggaagcagaggttgcggtgagctgaatcatgccactgcactccagcctgggcaacagagcagcacttcgtctcaaaaaaaaaaaaaaaagatctcaaatgaattgggattgtattaagtaatgattaagtaatgtgattacagcaatcctcaagaaatatttcactgtggccagtaacaatgtgtaacagacctttaaacttctagagattttcctacaacatgtgtctcaggctgatgtgttttatttagtgcttctcttggaaatgtcttgcccctcgatactttatcattaaggtctttaaggcagggatcatgactctacttttttttttttttttttttttgggacggagtcttgctctgtcgcccaggctggagtgcagtggcacaatcttagctcactgcaacctccgtctcctgggttcacgccattctcctgcctcagcctcccgagtagctgggactacaggcggctgccaccacgcccggctaattttttatatttttagtagagacggggtttcaccgtgttagccaggatggtctcgatctcctgacctcgtgatccacccacctcggcctcccaaagtgctgggattacaggcttgagccaccacgcccggcctcatgactctacttctaatatctcatcatgtgctcttccactgaggcttctacttagagctacacaatctgggcagccatcctcagtgccttatctaccaacatgctcaatatggctttgcagggttcactgtctaccagcagggttcactatctaccaacatgctcaatatttctttgcagtcaggcagagcaggctttgcagttcaggcagggcagctggctgcaggccccagctgactcctggggatagaatgccaatatttcagacattgcagagatttgaggcaatgtacataaagccctccacatataactgatgcacaataaatgacagttaatattatgcaacaagaatttcctggggggttttataattaatttttatttgtgtgaagttttttccctcccttttactttaatcctttttggggggaaagcatcactagtcacagttcacggcagcctcgacctcccaggctcaagcaaccctcccacctcagcctcctgagtagctgaaaccacaggtgtgtgccaccacacctgactaatttatttttattttctaatgaaacagaatcttgccatattgcccaggctgatcttaaactcatgggctcaagcgatcctcctgcctcagtcttccaaagtgccgggattatagatgtgagccactgcactcagcctttttttttttttttttaattgtagatagcataaaacctactgttttaaccatgcttaagtgtacaattcagtggcattaagtacattcacagtgttgtgcagccatcgccattatgctgcattattttcagaactttttcattattctaaactgaaactttgtatccattgaacactaactcccaattcccccagtccctggtaacctccattctactttctgtcactgtgagtttgactattctaagtacctaatttaagtggaatcatacagtatttgtccttttgtgtcaggcttatttcactttgcatgatgttttcaaggttcatccatgttgtaacctgtcagaatttaatttcttttcaggatgaaataatgttttattatatacagtcacaccattttgtttatccattcatctattgatgtcttctctctcttacagctcttcaaattcatcaattttaacaggaccatgagccagctctcgacaaccatgtctcgatgtgccaaagacctgtttggctttgctattatgttcttcattattttcctagcgtatgctcagttggcataccttgtctttggcactcaggtcgatgacttcagtactttccaagagtgtatgtaagtatatatgaaattaagaagaaaaatttaatcagagttgtcactgcttctcaagaataaatcttcatatgaggttgctatatgaccaccaattatttaaaaccagttattttaagtaagaattaattaccttttcccaaaacattgatctacccatgcaaagaagacaatgcatcctgaaatgctgatgcttaagatagcagcccaaagtagtaaaatacagttaacagacataggaaaccaacactgttctgaagactgagtttttctttgcaccaaatgcagatggtagcttctagaaggctgtttgcctatattcttactcctgttgaatattgttgccatatatttagaacttcaagttattttctaaggaaaaaaacaagatatttctaatattctaaggtaaactcagaccagtacaagaattttcagtttttttttccaaagatcccaaatgtgaaataaaacaacaaaaagcagccagtgtcagatttctatgccatttagaaaggagttagtttaaaaaggaatggaagtaatagggttttgtgcatagatatctcgaattaatattgctgttgataaaagtgattttgctaagacccagcactgacaacacttggccactttgatcccattttaagtacttgtcagaatattggatctttgaactcaaaccattttgggtttttggggtttttttgttttgtttttttttgttttgttttgtttttgaggcacggtcttgctctgttgcccaggctggagtgcagtggtgcaatcatagctcactgcagccttgaactcctaggctcaagcaatcctgctgcctcagcctgctgagtagctgggactacaagtgtatgccaccatgcctggccaatttttaactttttttatgagaagggatctcactgtgtagcccagggtggtattgaactccagggcctcacactgtcctctcacctcagcttccaaaagtactgggattacaggcatgagccaccacaccaggccctgttgttttttttttaaagaaatttttaactttagaccgagggtgactgttgtcaaggtttagggttaagatgttttacctagattatgtgttgaaatgttatagccaattgctttataagttattgaataataattgtattttctttttttttttttttttgagatggagtctcgttccatcgcccaagctagagtgcagcggtgtaatctcagctcactgcaacctctgcctcccgggttcaagcgattctcctgcctcagcctcccgaatagctgagattatgggcgcacgccaccaagcccagctaatttttgtatttttagtagagacggggtttcactatattggccaggctgttctcgaactcctgacctcgtgatccgcccgcctcggcctcccaaagtgctgggattacagacgtgatccaccgtgcccagcttgtgttttctttttaaccaaatggaaataacctctgtagcatgaaagcattttattattattgcagaaggctttaattgctgatacaagtagcaagactttgtaaatgggattgacaattttctgttattcggcagctacctatactgctaaaaggtccaaaaataatgaaatcatctttaagaaatgttgcatcaactagtggacattctttgtttttgtattgtggtgttttgttttatttttatagcttcactcaattccgtatcattttgggcgatatcaactttgcagagattgaggaagctaatcgagttttgggaccaatttatttcactacatttgtgttctttatgttcttcattcttttggtatgtacatttttatttatagtgaggttcaatttaaacttcgtaaatccttgtcttctcttttctctcacactttatgtcctatcaattttaaataaagacccaggaagtagaaaaaagtgtggatgttggaaaacttattttccttttattaattcacagttttgagactcatatcaaatgtcttttctgtggtctttcattgatccatgtatatgtgtctattcaatgcaaaaaaaattagatctcttccatggtctttcatttctctctctatatatgtatctattccatgcaaaaaagaaattagatcaagtacaaatttataaagatacctaaaatagtgctttgcctaaaaagtagaatatgcttacatgctttttaaactcatatgtcagcactttcgtagtcacttgctagcatgacttttctctctttcttcttttctttttaaaaaataagaacggaaaagcaagctagatctaagatgtcgagtaatagttgagtgaatcattgcatgtcaaaattaggatattctgttttaaattatttatatcccattcatctagagactgcctacagagaatattcaaataattaagtttaaaactaaatgtaacaatgaatggaaattgcattaaaattattttcaaaaataatttttttattctcttgatttggtacaaatgaacatttttaatgtttttgccctaagtcaattaagtttttttaaggtgttttgttctttttcttaacatttatatattcaattgtctactgagaaggtgttaagccagcttaatttaggcaatatttttcatctaaacactaacagtcatcttaagaacaattttcttaagaaaataacattttttccatttcagtaaattgtgtaaagatcccttgaggaaggttaagtgatcacattttcagtaattcagtgtaataactctaaagtcagtccaggtattactggttaagtatatggtatttattgattgggtattagatgtactgtattaatttcctgtttaaaaaaaattttttttccggggagacacagcctctggtgtaaaacaaaggtgtgttccctagctgtactttaacaggactgaaaaggtcaggaatatcattcaagttcatatgtatcttgctgtatgcatggtttatggctcatttttaaacttacacctcttaagcttcttcttcctatcatatattaaaacaatggagagaagaataagcctctgttactctaccattgatagtacttcggattctagagtacctgaatctctactaagaaggcaaaaaccaggaattgagagtcctgcacctgacccttcagttgatctcaggccacctagttttctccgtttatcaatctgccaaacaaggatggatagagtcgtggcaactggaaaggctcaaatgtggaattgtttgaatgtggtcctttagtaggcagccatcttaccagatctagagtattcagtcatcttaccagatcagtcaccagaacatgaaaagaagctcttagtttctatctttatactaaaattgtttttttgtacgactgcacaaaaaagaattgctctccttgcacctcccagagatataggtggatagatacatacatacgtacatacatacatacatacatacatacatacatacatagacacatacatagatagaagtctactttcaatacaaacctgtcttttaaggaaatgacaagctgagcatagggttggccacctttctgagccgattgcctggtattagtttattgcccctgtttagcaagaaggcacagtgttaagaagtggctcagctgaaccaggataaccccactcttcccccacatcaacaggaaagacatcctggtgcagatgtccatctgataattcagggaacctcgggagacaggatggagaggagggtgagctagcttcctcttcccacaccttcaagagcctttctcaagcactttctattttttgaaatctctttagaggtcccagactttgatctgtttcaattaaggtattggcaggcattagttaacagccacttggaagcaaaaatagaacattagatccctgagttggaagagagaaggtagaaggtgttacttggactgcaattatctgcacttggaattgagcatttagtcaaaaacttatatgtattctatattctattctcatttctgctacagaattgtaaacaatattcttccttaatacagaaattcatagcccactaaaataagagcgttctcatttgttcatttctcaatcatttaataagtatttactaagccactatatccatatatatatatatatcatatatactgtataatacacactgtagtgttttttgtggattgtgtactatgaggtagtatgttagatactgccagtactggggtaaggaaaacagcctgattaggcccttacgaagattcctcagacttgtggggaaaacagacattatcaaatagaaatacttgcaaaccacagttatgtgttaaaaaggaaaaacaaagtaaaaaaaaagttggtgggggggaacctgatctcctggatacagtgcttcgagaaagtttgttgttggaaatgcaaaccattactactgtggaagggaaaggtcagaaaaatgaactcaccattactgaatagtaatagtagctatcaattaggtggcacttacctgcatcaggacctgtcctgagcactttacatagattgtctcactaaccagcccaacaaatatgtaagggagatactattatttttcccattttattaatgtaaaacaattaaataattctttaaaattagacttagaaaagtggagcaacaatcttagcagtgctaggactgaaatccaagtttgcttgactccaaagtctatctctcttccagaaactttttctttactatctgcctagtaggcctgctgtattcctatttgcaacagccttttaaactctttaaaaatgtgtcctgtaaatttcatatatgattatacaaaaaaacttggaataagcatacaattctacttatctgtgttaactgttgaaatttgaagagctttttggaattctatacccttcagtagtgtatgtaaaagtttctaaatatagagaacatagataagcaaaaataatattaaataaaataatcgcaccattagtaggtaaatatactaatattttgttgtattttattcttgtatgttttcacaaagtatatcataaaatttttcctgtggcatgacttaacggagaaaataatcttcccaaaacatgtggcagcaaaactgttaatttattacatcaggctgggcacagtggctcacgcttgcaatcccagcactttgggaagccgaggcgggcagatcacttgaggccaggagttcgagaccagcctggccaacgtggtgaaacactgtctctactaaaaatacaacagttagccaggtgtagtggcacatgcctgtaatcccagctactcaggaggctgagactcaagaattgcttgaacccaggaggcagaggttgcagtaagctgaggtcgcgcccctgcactccagcctgggcaacacagtgagactctgtctcaaaaaaaaaaatttttttttaaataaataaataataaatttatgtcttcataaagcactcagattaggaaaaaaaggataaacaaaaaggcatgtgtcatttttttgattgataattccaaattatgtttcttcctttaatttttgccctcctttcatttacaaacagaatatgtttttggctatcatcaatgatacttactctgaagtgaaatctgacttggcacagcagaaagctgaaatggaactctcagatcttatcagaaaggtaggaaaaaccttaattctcagaattcttctgtttctgacataaaatgagcattgtttcacccagattttcaaatcaacattgatccattgaaattgtttgaaataaagaatacattgctatatttcaggaataatttaaatgttccctatcttggagtcttgatggatatactgctatcttgaattttaattctgggaatccttttatgccctggaattaaattctcaacaatcttttgacactttaagagctgagctgaaggttcatcaccttcattattttgacatctcctgtagctggctctcacttcaggatcctgagttgagaataaactagaagggaagattatataaagggatttccacctcttctgtctcaattaccattttaaaaaaataaaaagttttagaggaaaacacttagtagttcaccctttacccttgaccttccacggcagttttaaaataagcaaaggaaaagattcatgaattcaggccatagcctggggcctgagaacttttacttatgcaccttctcaggaagggtttcattgttaaatagaagggcaggacaggaaagttgggcctctttgttcttctcaatgtaacttctttatttggtttaaagtataaaatgtatacaacaacaaataaccacatttaaaatacacagtttgtttcccaacatcattttgctaagtcatagtggctccttaactgtaatttttttttttattagtccaagccttaggattatgttatctgtgatatatgttataatagaaaacttaagcctcttaaaacaaagtccttgggatgggacctaagattcacattatcttgattccgcataacagttgcttacattttagcaaatctccagtgtgtatgcaagcactcctcacttggcacaattctgatacacacaaactttggttaccccagttttgttatgtaacaccaccttcaacaacacagttcaaatttcagttatcatagtatattaactctgagtaacagcacaaagtacaaactccactgctagctcttcagtgtatagatcagttacctgagtaacagatgtgcaggctgagcaggctcactggtcagtcatgacactattttcagtctgttactaattggtcactgagcatctgctatccaattcacaaacaaagaaagcatgtagtgttgcttccttgtgttccagtaataagcccatgtgacattttacaaaaatggataattgaaaaagagaatgggtgcagtggctcacgcatgttgggaggccaaggcggatggatcacctgaggtcaggagttcgaaaccagcctggccaacatggtgaaaccccatctctactaaaaatacaaaattagctgagtgtggcgacagatgcctgtaatcccagctactggggaggctgaggcaggagaatcgcttgcacctaggaggcggaggttgcagtgagccgagatcgtgccattgcactccagcctgggtaacaagagcgaaactccgtctcaaaaaaaaaaaagaaaagaaaagaaaagaaaaagagaatgggctagcaaagaaatgaaaaatggtaacactggaagtgaaaatcaaaacagagtaatggatttatagaagaaatagctgagtgaagaagaaataggagtgttgacactgcgatcattcaagagatccagatatggagccagaagaacttagggcaggtctatcaacttaaatgaggaaaatagctgtgataaaacagatgaagatgtcttgaggaaatgatgcctgcaaaaaacttcacattaagggaagtcttattagagatatttcacaataatgaaagtacaaaagaaaaaatgttggggctggacatggtggcttactcctgtaatcccagcactttgagaggccaaggtgggtggatcacttgaggccaggaagtcgagacaagcctgatcaacatgatgaaaccccgtctctattaaaaatacaaaaattagccagacatgatggtgcacacctgtaattccagctactcaagtggctgaggcacgagaattgcttgaaccagggaggcggaggttgcagtaagctgagattgcaccactgcaatccagccaggtgacagggtaagactgtgtctcaaaaataaataaaagaaaaatatgttggaagctcatccacatttaagaaggaatatgacaattcactaatgcatagaaaagaagttcactccacattgtaaagtgtacagtgtaatattatacaatgaaaacaaggcaagtgctgtttaaactactctggatacattttttacaaagaaataaaacactttagtttttaatgtttctaatgttttacattttagtgtattaaatcaatattagttttcttcttttttaagctccctatacatttataactgacactaagggagtgtttaatgttttgattaaaagttgtaaagatcacagaacaattgtaattcttcccactgattattcagatcattttgcacaatttcagcttgcatggtcacttacagtgccgcactatgtgcaaagcaaggtcaggtctaaagttcgctaatgaaaaatcctcggccaggggcagtggctcacccctgtaatcctagcactttggaaggcgaggcaggcagatcgcttgagctcaggagttcaacaccagcctgggcaacatggtgagaccctgtctctacaaaaaaaaaaaaatagcaaggcgtggtgactcacacctgtagtcccagctacttgttgggggctgagttgggaaaatcacttgagctcaggaggtcgaggctgcagtgagccagaatcacgccactgcccttctgcctgggtgacagagtaagatcctgtctcaaaaaaagggaaaatcctcatctacatttcactgggttttttgtttgtttgtttgtttgtttatacacacttaaggaaattactgtctagaagatagataatataaaaaataaaaatgcaattcatgattcgggtttcttggtattcctaagaactgttgcacagtactttatgctctgaggcagacagctatagcatatatagtaatttttgtttctatcacataaacttgaatacacatatgagtaaaagacctttagttcttcatgacttactgaaagaccctgactttttccatgtaactgttccacaagtgttttatggaaaactggatacattaattcttcattcatccagcacgtacttgttgaatggccaatgtacaccaggtttgtagtagttactactgtgaatggaaagtaaaacagatgcaaaaggagaatacactaaaccaagtcttttattttttctctctctgatagggctaccataaagctttggtcaaactaaaactgaaaaaaaataccgtggatgacatttcagagagtctgcggcaaggaggaggcaagttaaactttgacgaacttcgacaagatctcaaagggtgagaatcatgcttcctgaggttctgaaaaattcctgcttctaaagataaattcctggtgataagagtatttctagcccaagggctcatacagatactttttttttttttttccagaggcaggtatctttctggaacatgttataagaggaaaacttgcccccatttggtgatttctcctttcctcctgcattttgatgtctctgtgttgagggtgaactgggtacaaggaatgatttttatctgtatcctctctctaatttcaggaagggccatactgatgcagagattgaggcaatattcacaaagtacgaccaagatggagaccaagaactgaccgaacatgaacatcagcagatgagagacgacttggagaaagagagggtgggtctggtttaggagaaccggatttgatttggtacctacaacaccacagatgtatcaaacactatagaagtagtgggttattgagtctcttgcccattccccaccacactctctctctctctcagtcggtttatgtgttagtaccctgtttattccagaaagaatatataacacaattatgtataaaaatgggtggttagcatgatataaaaacgtcaaaatgaaaagcaagcaaaacaaaagtaaaaataatggattattaatgaagcttaaaaatgcattcataaaaacacatatgcttattaagattgggctacaaattgggccctaagcttgctggtaatcagcttgaaaagagaagcctgattagctgcagagtccacaatgtccgtgagagtgaagaaaacaaaaaatgacttaccaagagatgtgaaattattctggttagttagtggctatttaaattgttaacttttttttcttttttttttttttttgagatggagtcttgctctgcctcccaggctggagtgcagtggcacaatcgcgactcactgcaacctccacctcccgggttcaagcgattctcttgcctcagcctcccaagtagctaggactacaggcacatgccatcatgcccggctaatttttgtatttgtagtagatatggggtttcaccatgttggtctcaaactcctgactgcaagcaatctgcccaccttggcctcccaaagtggtgggattacaggcagtagccaccgtgcctttcctaaattattaacatttataataaaattaacagccgccttccatttgaatactttttacaaaatagttaaaaataaacataagtgggcttttatagtcagaaaaaaaaattcaaagctttaccattaactttcaaaaataaatggttagacagcaacaacaaaaatctgtggtaactgaggtacagagaacacagatgaatgttattacaaaagccactttcctatgagaagtctaggacagtggtttctaaatgccactccacagacagtgctagtaggtgacagacttctccagtcacagtgaaatttaagcataaagaaaatgaggaaaatttttacaaggctctatttagacaaagttcttattctgacattacatctttcctactttggagctgttgaatgtattatcttttatgaaaagaaggcgatccaggttgagcatccctaacccaaatatgtgagtctgaaatgctccaaaacctaaaacttcttgagcacaaacatgatagtcaaaggtcatgcttaaaggaaatgctgtcattggagcagtttggattttgggttttcagattagggatgctgaaccagtaagtataatgcaaacattccaaaatatttttgaaaatcccaaatccaaaacacttctgatcccaagtatttcaaataagggatactcaacctgtaatatatttcttcatttctttatttattttattattattttaagatggctcatggcccactgcagcctcaaactcctaggctcaagtgatcttccgacctcaacctcccaggtagctcaggtagctgggactgcaggcatgcatcaccatgcctggctaattttttaaaaaattttttgtggaggcagagtctcaccttgctgcccaggccagtctcaaactcctggcttcaagcagtactcctgcctcagcctcccaaagtattaggattacaggtgtgaccactatgcctggcccatatttcttcatttagttttttctttgcctgctgtgtttttaatgttctttcttgttcaaacaaaaagttggctattccttgctgttagttaaatttgccaatctatgaaactgaaaaatgcaggagtcccagcctggtgttaaatacaaagaaatcccaggtaaatggcatgcacccagttcctgcttgcccaagtccttggtgaggcttctgtggggtctcagtgttctgctcctcactcagtgaccccttgttcttcaggaggacctggatttggatcacagttctttaccacgtcccatgagcagccgaagtttccctcgaagcctggatgactctgaggaggatgacgatgaagatagcggacatagctccagaaggaggggaagcatttctagtggcgtttcttacgaagagtttcaagtgtaagtataaaggaattggcagaatttgcgttgacaagagtccacatgagaccaggcagttccctcatctctctgaattcactcctttccattactaatcatccagcttttaaaaataacttatactggccagacgcagtggctcatgcctgtaatcccaccactttgggaggccaaagcaggcaggtcatgaggtcagaagttcgagacgagcctggccaacatagtgaaaccccatctctattaaaaatacaaaaaattagctgggcatggtggtgggcacctgtaatcctagctacttgggaggctgaggcaagagaattgcttgaacccgggaggcggaggttacagagagccgagatggctccactgcacaccagcctgggcgacagtgcaagactctgtctcaaaaaaaaaaaaacttgtcaattggtgttttgtttcttacataatatgtttactataaaaattagcaaataagagcaaaagaaaacattaacatttcacatatttctaccaatgaaaaatgtttattaatatatcagtgtttgtgtctctatttgcatgtgtttatcaatgtttctatatatttttatggcctaacatatggttcgtcctgaagaatgttccttgtgcatttgagaagaatgaatattctgctgttcaagtgttctgtagatgtttgttaggtctagtttgtttacagttttattcaggtctcccatttcctggttgatcttagatgtgcctagatgtggtattcacgtttgaaagtggggtattaaagtctccaagtattattagttggagttcatcccttcaattctgtaaggttttgctttgtgtattttggaactctgttgttgggtgcatacatatttataactagtatattttcctaatatattgaccctattttctctctcaacttaatgaggctaaagaaaaaaaagaattgaccctgttttcattacaagatgttatccactttatctctagtaaaattctttgttttaagtattttttgtttgatattactgtaaccactccagctttcttttggttgctgtttgcatgataaatctttttccatccttttactttaaacttatttatatctttcagtctgaagtatgtctctcctgtagacagcatataattggatcttatctttttatccagtttgacaatttctgtttttgattagattgcttaatccattcatttaatgttatcattgatgtagttgatttctgtctgctattttattttttgttttctagcttacttttttttgttcctctttcactgctttcttgtacattaagtgaatattttcaagtataacatttaaatttttttaatgatttttcattttttttagtcaggagttgctctaagacttagtttatacaattaagttatgaaaaattacttcagatatatattaactgaatccagtgagatatagaaatcatttctatttagcttttttcctcttccctctttttgtgctatatattcatctatctatatgtatatatagtcatctacatatgttgcaaatcacattgttagaacaatgttacatttttataacacactgtgtaatatatagtatataattttatatctcttaatgaagctgagagaagaggaagtatatatttataaatgtgtatattaacctacttttttaccatttctaattctcttcttttgctcctctggattcaagttatcatctgtcgtcattctctttctccgatacagctttgctactgcctacctcctattattgtcaaatatattacatttctattatagaccttcagatccaattatgtacatatttttacacaactgctttttaaatcagttaaggaacaaaaggagaaatgtacatatatactatattttatacctacacagttatctttaccagtgttctttgccttttcatgtggattctgattactatctggagtcacttgctttcagcataaagaatttcctttagtattttttgtaaagcaggtttgctagcaatgaattctttcattttttgtttatctgagaatgtttttctttctccttcatttcctctggcttgtattgtttctgatgagaagacaggtgctaattttactgtggtccccttgtacatgatgactcaattttctctcaccactttcaagatttttttgcttttgtctttcattatttttactgtgatatgtctgggtataaatctctgagttcatcctacttagaaagtgttttttctgcttctttcactttctcttctcctttgggacccgcattatgcatatgcttaggggtatcacatatttctcttaggctctgttcgtcgtcattttttttccctctctgttcctcagagtgcatagtctgtattgatgtatcttcaagttcactgacttgttcttctgtcagcttgcttaaatctctgttgagctcctctagttatttatttatttatttatttattttatatatatatatatatatatatatatagagagagagagagagagagagagagaaagagagagagagagagacagggagacagggtctcactccatcacccaggctggagtacagtggtgtaatcatggctctctacagcctgaacacctgggctcaaatgatcctcctgcctcaacctcccaagtagctaggactatgggcacatgctgccatgcctggctaatttttaaaaaaaaatttgtagagatggcatcttgttatgttgcccaggttggtctcaaactcccggcctcaagtgatccttccgcctcggcctcccaaagtgctggggtcacaggtatgagccaccgcacttagcctgaattttttatttattatacttttcaactccagaatttctatttggttctttttagcaatctctgtctctttattctgtatttgatgatatcctgtatttgatgagacaatgtcatcataacttccttttttttttaagagatagggtctctctctgtcacccaggctagagtgcagtggcatgatcctagctcactgcagcctcgaactcctgaactcaagcaatcctcccacctcagcctcctaagtacctgacactacaggcatgagccactgtacccagctaatttttattttttgtagagatggggtctaagttgcccaggctggtctcaaactcctgggctcaagtgatcctccctgctcaggctcccaaagtgctgggattacaggcatgaaccactggacccagcctcctttatttctttaaccatggtggggttttttattatttgtttgtttgttttaacctgtttgaatatatttataataggtaccttcaggtctttgtctgctaagtctgacatctgggccctctcaaagacagtttcagttgtctttttttttttcttcttgtgtatgggaacattttcctgtttttttgttttgttttttgttgttttgttttgttgttgttgttgttgttaaagattggacatgttagataatatattgtaggaactctggctagtgattccctttcctccccaggacttgtttttatttctctttgcttgtttactttttcatacagtctgtttccccacagtgtctgcctctgactttattccttagagggcgcagctgtggccatgtacgtagtcactctgggaagacagtggttttagcagcgtgctcattaactttctctgacctctttgttatacctcctgcctctgtggatattagacccagttattacatttcattgtttactgattggtctattgttttccagaatgccttgggatgtaatttgctccacagtctgatccacttaaattcaggcctctttgcagggctagttttagaggccagtctttttttttttttttttttttttttgagacagagtctcactctgttgcccaggccggaatgtagtggtgcagtctcagctcactgcagcctcaatctctcaggctcaagcaatcttcccacctcagcttctcaagtagctaggactacaggtgcacgccaccatgcctggttaatttttgcattttttatagagatggggttttaccatgttgccccggctggtctcaaactcctgagttcaagcaatccacccacctcagcctcacaaaaggccgggattacagatatgcaccaccacgcctggcccctgaggccagtctttaactgtcttcttagctgtctctttccctggttctctctggtgaactagctggtaatttgtttatctcataaggctaccagattccttgtaaatgcttatccccacaatctccattgttttcaagagcatcgttagtctttaatttcctcacgcttcattccaaataaagtccattcacttgagaagagttctatattcctatgacctgtgtctccccatgagcaaaactgctactgctttacagagccagggacagtggcccacctctctgtggcatcctgctttatgaacaagtcactgggctcagatggcagtctctgattttctcaccttgcttcttctggcatggaaactccaccctacaagtgggaactgagtggaagaagagagccccattccccttagccactcttaacaggattagaacctctgcaacatgcatttaagaatgggaacaggctgggcacagtggctcacgcctgtaatcctagcactttgggaggtcaaggcaggaggattgcttgagccaggagttcaggaccagcctgtgcaacatggtgagaccctcatctctacaagaaatagaaaaattaactggtgggttgtgtatacctgtaatcccagctactcgggagcctgaagtgggaggattgcttgaacctggaggcagaggttgcagtgagtcaagattacaccattgcacgccagcctgggcaacagagtgagactctgtctcaaaaaaaaaaaaaaaaaaagagtaggaacattgaggctgggcatggtggctcatgcctgtaatcctagcactatgggaggccaaggcaggagtatcacttgacgctaggagttcaagaccaacctgggtaacatagcgagactttgtctctattaaaaaaaaaaaaaaaaaaggtgtaaaaaacttttgtaacaagagtgggaaagccgggcacagtggctcacacctgtaatcccagcactttggaaggccaaggcaggcaggcggatcacctgaggtcaggagtttgagaccagcctggccaacgtggtgaaaccccatctctactaaaaaatagaaaaattatctgggcatggtggtgcacacctgtagtcccagctactcgggaggctgaggcaggagaatcacttgaacctacgaggcagaagttgcagtaagccaagatcacgccactgcactccagcctgggcgacagagcaagactctgtctcaaaaaaaaaaaaagagtggaaatgttaggatgagaaatgctggcagcctgcccctccctgggagatactgtagccctagactggaagttgggggaggagggagccctgtgctcttagctgcacccatgtgaagttgtgcttctatcacatgagctggggacaggagagaaggctcagattatggcttcagtgccacagaatctcttcatactaaaatttagtagattttcttgaataaatgctttttcatttgctgtacacccttaaaagtttctagaaatttttaatatttgagttttaaaaaataattttcaacagttacagttatttcactaaagagagagtctacagaaccctcttgccaccattgcagaggttgtctttggcttacgagtttttaaagtatttgtatacattttttaagttcaaaataatagaattgtaagtgaacatgctgttttcatactgtttttcaagctttatttaatatattgtaaatctaattctattttattaaatagtctgccacagtataatgtctgatgtctccttagaattttattgtatggatgaacaatgattatttaatttcctaccaattgttgggtgttttttgtttgtttgtttgtttttgagactgggtctcactctgtcacccaggctggagtgcaggagtgcggtggaatgatcacggctcactgcagcctcaacatcccaaggctcaggtgatccttccacctcagcctcgcaagtagctgggagtacaggcacatgccaccatgcccacctattttttagagatgaagttttgccatcctgcccaggctggctcgaactcctggcctcaagcgatctgcacacttccgcctcccaaaatgccaggattacaggcgtgagccatcatgccctacccccccatcaattgtttgatgtagccatttttcaatgatccgcgattaagaagcagcactcttttatagccaaaaattacacatatataaaattttcctttagaaaatgttctaaaaatggaatgtctaactaaagggttaggcatacattcttaagacttctgatacgcgctgacttgcaggaaagttgtttcagttaacactcctaccagcggcatccgagagttaatctgtaaagcttgagacaacttagaaagtgtttcaaatgattgtgttgcttaagaaaaaaatcttagcacttccttttgaaaagccagtggggctgaaaagacaatgacaagcactttgtccctctgtactgtgttttccttgcagcctggtgagacgagtggaccggatggagcattccatcggcagcatagtgtccaagattgacgccgtgatcgtgaagctagagattatggagcgagccaaactgaagaggagggaggtgctgggaaggctgttggatggggtggccgaggtcagtagtcatgagctgaaaacaccgctgctgagcatggtgttattaatgaaaatatatgttgctgacagttgtatttgaagtattgaagaagagtaaaaaaaatttacgtttatagaaattcacaatgatgtttccatttactctcattttcagatttttttctctgaaacagaaacactctttctataaaatctcttgctataaaacatcaatgtagtcatattgtctaacccttaggctgagatgtttatctttctccataactacagataaaattataatctggaggtgttactttcttaatactccatatgctaatggtcctgccttcactgcagggtagaattaagtgaaaaattactccagcaactctgagatttgctattatatgctgtaaatctccagccttaccaaactacagattatttggtccctggacttcctaaggcatttccttctactgcccccaacaccagtttctttttccctttttaggatgaaaggctgggtcgtgacagtgaaatccatagggaacagatggaacggctagtacgtgaagagttggaacgctgggaatccgatgatgcagcttcccagatcagtcatggtttaggcacgccagtgggactaaatggtcaacctcgccccagaagctcccgcccatcttcctcccaatctacagaaggcatggaaggtgcaggtggaaatgggagttctaatgtccacgtatgatatgtgtgtttcagtatgtgtgtttctaataagtgaggaagtggctgtcctgaattgctgtaacaagcacactatttatatgccctgaccaccataggatgctagtctttgtgaccgattgctaatcttctgcactttaatttattttatataaactttacccatggttcaaagatttttttttctttttctcatataagaaa

SEQ ID NO: 243 - DNA, 23754 bp, Homo sapiens
  source 1..23754: mol_type = genomic DNA
acagttcaacaatatagctgctgtcacagtattttttgtctggattaaggtaatttataaatttcatgttctacattttaaataatattttctttaaaaaaaatgagttccacaaaatcatggaatacttgaatttgaaattcaagtgaccagccaaagctgctcaatatttactttgagacagggtctcactctgtcacccaggctggagtgcagtggtatgattacagctcattgcagcctcgacttcccaggcccaagcgatcctcccaccttatcctccgaagtcactgggactacaggcatgtgccaccatacccggctaatttttaaattttttcgtagagacaaggtctcattatgttgcccaggctggttttgaactcctgggttcaagcaatcctcccacctcagcctcccaaagtgctgggattacaggcatgagccaccgtgccaggcctcatattttacatataaagtaaactattgagactcatgtgatcattcctctcactgtcaatgacatacttctgctatctgaattagtgcaagatcagtccctataggttttgtttaacaaatgcagtaagaggcctttcagtgtgttagctgggcctggggccccaggctgctaacagatgagatgaacaggtgaaggaaaaggaacttagagaaagagagggaaggagcaggtggagggaaggggagagttgctgcacttggaaatgcttgctagaagggatcgcctcttttccaggtagaggctgtaagggaagctttacctagaattaaggttggaacagacactgcttccaaatagttccttgctcactattttccttattgtcccaagatataatgtgcatttccatgtgtgtgaaaggttatgacatttcatatacaacaagcctcaattctggagatgcaggaaatttcaataattctcaggcagcagctgccattcggtcaccagcacaggctctgattgtgctgtccagacagtaagtactagccacatgtgcctatttaaattcaaatttaaattagttaagcttaaatacaattaaaaacgcagttccttggtcctactggccacacattaagtgttcaatggctactgtctaggacagtggaaatgtagaacatttccatcatcacagaacgttctcttgaaaagcactgttctggaaggtacttacccgttatgtacttttctgagttggtattcatacctagaagacctgaggtttatcacaagacatagacttggaccaggcgcagtggctcatgcctgtaattccagcattttgggaggccgagataggtcccctgagcccaggagtctgataccagcctgggcaacatggcaaaacctcatctctactaaaaatacaaaaattagctgggggtggtggcacgtgcctgtagtcctagctacttaggaggcttaggcgggaggattgcttgaatccagaaggcggagggtgcagtgagccaagatcgcaccgctgcactccagcctgggcaacagagtgagaccctgtctcaaaaaaaaaaaaaaaatgcatagactttatcctgtatttctcatgctatttatttattgacatgcttgttcaagagaaaccatcactaaagcacaaaaccttgatcataacatagtaataataatcaaacagcaaaaataataatagtaataagaatgttctgtggtgatggaaatgttctatattttcattgtcctagacagtagccactaaccatgtataggcatggaacacttaacatgtggctagtaggaccaagggactgaatttttaattgtatttaatcttacttaatttaaatttgaatttagatatccacacatgtttggatagcacagtcagagcctgtgctggtgaaaggatggcaggtgctgcctgagaattactgaagtttccttgattattattagtttaataataataatcaagatagtaataataatcaagatagtaataataatcaagatctcagctgggcacagtggctcacgcactttgggaggctgaggcgggcagatcacctgacgtcgggtttgagaccagcctggccaacatggtgaaaccctgtccctactaaaaatacaaaaaaaaaaaattagctgggtgtggtggcacgtgcctatgatcccagctacttgggaatctgaggcaggagaattgcttgaacccaggaagcagaggttgcggtgagctgaatcatgccactgcactccagcctgggcaacagagcagcacttcgtctcaaaaaaaaaaaaaaaagatctcaaatgaattgggattgtattaagtaatgattaagtaatgtgattacagcaatcctcaagaaatatttcactgtggccagtaacaatgtgtaacagacctttaaacttctagagattttcctacaacatgtgtctcaggctgatgtgttttatttagtgcttctcttggaaatgtcttgcccctcgatactttatcattaaggtctttaaggcagggatcatgactctacttttttttttttttttttttttgggacggagtcttgctctgtcgcccaggctggagtgcagtggcacaatcttagctcactgcaacctccgtctcctgggttcacgccattctcctgcctcagcctcccgagtagctgggactacaggcggctgccaccacgcccggctaattttttatatttttagtagagacggggtttcaccgtgttagccaggatggtctcgatctcctgacctcgtgatccacccacctcggcctcccaaagtgctgggattacaggcttgagccaccacgcccggcctcatgactctacttctaatatctcatcatgtgctcttccactgaggcttctacttagagctacacaatctgggcagccatcctcagtgccttatctaccaacatgctcaatatggctttgcagggttcactgtctaccagcagggttcactatctaccaacatgctcaatatttctttgcagtcaggcagagcaggctttgcagttcaggcagggcagctggctgcaggccccagctgactcctggggatagaatgccaatatttcagacattgcagagatttgaggcaatgtacataaagccctccacatataactgatgcacaataaatgacagttaatattatgcaacaagaatttcctggggggttttataattaatttttatttgtgtgaagttttttccctcccttttactttaatcctttttggggggaaagcatcactagtcacagttcacggcagcctcgacctcccaggctcaagcaaccctcccacctcagcctcctgagtagctgaaaccacaggtgtgtgccaccacacctgactaatttatttttattttctaatgaaacagaatcttgccatattgcccaggctgatcttaaactcatgggctcaagcgatcctcctgcctcagtcttccaaagtgccgggattatagatgtgagccactgcactcagcctttttttttttttttttaattgtagatagcataaaacctactgttttaaccatgcttaagtgtacaattcagtggcattaagtacattcacagtgttgtgcagccatcgccattatgctgcattattttcagaactttttcattattctaaactgaaactttgtatccattgaacactaactcccaattcccccagtccctggtaacctccattctactttctgtcactgtgagtttgactattctaagtacctaatttaagtggaatcatacagtatttgtccttttgtgtcaggcttatttcactttgcatgatgttttcaaggttcatccatgttgtaacctgtcagaatttaatttcttttcaggatgaaataatgttttattatatacagtcacaccattttgtttatccattcatctattgatgtcttctctctcttacagctcttcaaattcatcaattttaacaggaccatgagccagctctcgacaaccatgtctcgatgtgccaaagacctgtttggctttgctattatgttcttcattattttcctagcgtatgctcagttggcataccttgtctttggcactcaggtcgatgacttcagtactttccaagagtgtatgtaagtatatatgaaattaagaagaaaaatttaatcagagttgtcactgcttctcaagaataaatcttcatatgaggttgctatatgaccaccaattatttaaaaccagttattttaagtaagaattaattaccttttcccaaaacattgatctacccatgcaaagaagacaatgcatcctgaaatgctgatgcttaagatagcagcccaaagtagtaaaatacagttaacagacataggaaaccaacactgttctgaagactgagtttttctttgcaccaaatgcagatggtagcttctagaaggctgtttgcctatattcttactcctgttgaatattgttgccatatatttagaacttcaagttattttctaaggaaaaaaacaagatatttctaatattctaaggtaaactcagaccagtacaagaattttcagtttttttttccaaagatcccaaatgtgaaataaaacaacaaaaagcagccagtgtcagatttctatgccatttagaaaggagttagtttaaaaaggaatggaagtaatagggttttgtgcatagatatctcgaattaatattgctgttgataaaagtgattttgctaagacccagcactgacaacacttggccactttgatcccattttaagtacttgtcagaatattggatctttgaactcaaaccattttgggtttttggggtttttttgttttgtttttttttgttttgttttgtttttgaggcacggtcttgctctgttgcccaggctggagtgcagtggtgcaatcatagctcactgcagccttgaactcctaggctcaagcaatcctgctgcctcagcctgctgagtagctgggactacaagtgtatgccaccatgcctggccaatttttaactttttttatgagaagggatctcactgtgtagcccagggtggtattgaactccagggcctcacactgtcctctcacctcagcttccaaaagtactgggattacaggcatgagccaccacaccaggccctgttgttttttttttaaagaaatttttaactttagaccgagggtgactgttgtcaaggtttagggttaagatgttttacctagattatgtgttgaaatgttatagccaattgctttataagttattgaataataattgtattttctttttttttttttttttgagatggagtctcgttccatcgcccaagctagagtgcagcggtgtaatctcagctcactgcaacctctgcctcccgggttcaagcgattctcctgcctcagcctcccgaatagctgagattatgggcgcacgccaccaagcccagctaatttttgtatttttagtagagacggggtttcactatattggccaggctgttctcgaactcctgacctcgtgatccgcccgcctcggcctcccaaagtgctgggattacagacgtgatccaccgtgcccagcttgtgttttctttttaaccaaatggaaataacctctgtagcatgaaagcattttattattattgcagaaggctttaattgctgatacaagtagcaagactttgtaaatgggattgacaattttctgttattcggcagctacctatactgctaaaaggtccaaaaataatgaaatcatctttaagaaatgttgcatcaactagtggacattctttgtttttgtattgtggtgttttgttttatttttatagcttcactcaattccgtatcattttgggcgatatcaactttgcagagattgaggaagctaatcgagttttgggaccaatttatttcactacatttgtgttctttatgttcttcattcttttggtatgtacatttttatttatagtgaggttcaatttaaacttcgtaaatccttgtcttctcttttctctcacactttatgtcctatcaattttaaataaagacccaggaagtagaaaaaagtgtggatgttggaaaacttattttccttttattaattcacagttttgagactcatatcaaatgtcttttctgtggtctttcattgatccatgtatatgtgtctattcaatgcaaaaaaaattagatctcttccatggtctttcatttctctctctatatatgtatctattccatgcaaaaaagaaattagatcaagtacaaatttataaagatacctaaaatagtgctttgcctaaaaagtagaatatgcttacatgctttttaaactcatatgtcagcactttcgtagtcacttgctagcatgacttttctctctttcttcttttctttttaaaaaataagaacggaaaagcaagctagatctaagatgtcgagtaatagttgagtgaatcattgcatgtcaaaattaggatattctgttttaaattatttatatcccattcatctagagactgcctacagagaatattcaaataattaagtttaaaactaaatgtaacaatgaatggaaattgcattaaaattattttcaaaaataatttttttattctcttgatttggtacaaatgaacatttttaatgtttttgccctaagtcaattaagtttttttaaggtgttttgttctttttcttaacatttatatattcaattgtctactgagaaggtgttaagccagcttaatttaggcaatatttttcatctaaacactaacagtcatcttaagaacaattttcttaagaaaataacattttttccatttcagtaaattgtgtaaagatcccttgaggaaggttaagtgatcacattttcagtaattcagtgtaataactctaaagtcagtccaggtattactggttaagtatatggtatttattgattgggtattagatgtactgtattaatttcctgtttaaaaaaaattttttttccggggagacacagcctctggtgtaaaacaaaggtgtgttccctagctgtactttaacaggactgaaaaggtcaggaatatcattcaagttcatatgtatcttgctgtatgcatggtttatggctcatttttaaacttacacctcttaagcttcttcttcctatcatatattaaaacaatggagagaagaataagcctctgttactctaccattgatagtacttcggattctagagtacctgaatctctactaagaaggcaaaaaccaggaattgagagtcctgcacctgacccttcagttgatctcaggccacctagttttctccgtttatcaatctgccaaacaaggatggatagagtcgtggcaactggaaaggctcaaatgtggaattgtttgaatgtggtcctttagtaggcagccatcttaccagatctagagtattcagtcatcttaccagatcagtcaccagaacatgaaaagaagctcttagtttctatctttatactaaaattgtttttttgtacgactgcacaaaaaagaattgctctccttgcacctcccagagatataggtggatagatacatacatacgtacatacatacatacatacatacatacatacatacatagacacatacatagatagaagtctactttcaatacaaacctgtcttttaaggaaatgacaagctgagcatagggttggccacctttctgagccgattgcctggtattagtttattgcccctgtttagcaagaaggcacagtgttaagaagtggctcagctgaaccaggataaccccactcttcccccacatcaacaggaaagacatcctggtgcagatgtccatctgataattcagggaacctcgggagacaggatggagaggagggtgagctagcttcctcttcccacaccttcaagagcctttctcaagcactttctattttttgaaatctctttagaggtcccagactttgatctgtttcaattaaggtattggcaggcattagttaacagccacttggaagcaaaaatagaacattagatccctgagttggaagagagaaggtagaaggtgttacttggactgcaattatctgcacttggaattgagcatttagtcaaaaacttatatgtattctatattctattctcatttctgctacagaattgtaaacaatattcttccttaatacagaaattcatagcccactaaaataagagcgttctcatttgttcatttctcaatcatttaataagtatttactaagccactatatccatatatatatatatatcatatatactgtataatacacactgtagtgttttttgtggattgtgtactatgaggtagtatgttagatactgccagtactggggtaaggaaaacagcctgattaggcccttacgaagattcctcagacttgtggggaaaacagacattatcaaatagaaatacttgcaaaccacagttatgtgttaaaaaggaaaaacaaagtaaaaaaaaagttggtgggggggaacctgatctcctggatacagtgcttcgagaaagtttgttgttggaaatgcaaaccattactactgtggaagggaaaggtcagaaaaatgaactcaccattactgaatagtaatagtagctatcaattaggtggcacttacctgcatcaggacctgtcctgagcactttacatagattgtctcactaaccagcccaacaaatatgtaagggagatactattatttttcccattttattaatgtaaaacaattaaataattctttaaaattagacttagaaaagtggagcaacaatcttagcagtgctaggactgaaatccaagtttgcttgactccaaagtctatctctcttccagaaactttttctttactatctgcctagtaggcctgctgtattcctatttgcaacagccttttaaactctttaaaaatgtgtcctgtaaatttcatatatgattatacaaaaaaacttggaataagcatacaattctacttatctgtgttaactgttgaaatttgaagagctttttggaattctatacccttcagtagtgtatgtaaaagtttctaaatatagagaacatagataagcaaaaataatattaaataaaataatcgcaccattagtaggtaaatatactaatattttgttgtattttattcttgtatgttttcacaaagtatatcataaaatttttcctgtggcatgacttaacggagaaaataatcttcccaaaacatgtggcagcaaaactgttaatttattacatcaggctgggcacagtggctcacgcttgcaatcccagcactttgggaagccgaggcgggcagatcacttgaggccaggagttcgagaccagcctggccaacgtggtgaaacactgtctctactaaaaatacaacagttagccaggtgtagtggcacatgcctgtaatcccagctactcaggaggctgagactcaagaattgcttgaacccaggaggcagaggttgcagtaagctgaggtcgcgcccctgcactccagcctgggcaacacagtgagactctgtctcaaaaaaaaaaatttttttttaaataaataaataataaatttatgtcttcataaagcactcagattaggaaaaaaaggataaacaaaaaggcatgtgtcatttttttgattgataattccaaattatgtttcttcctttaatttttgccctcctttcatttacaaacagaatatgtttttggctatcatcaatgatacttactctgaagtgaaatctgacttggcacagcagaaagctgaaatggaactctcagatcttatcagaaaggtaggaaaaaccttaattctcagaattcttctgtttctgacataaaatgagcattgtttcacccagattttcaaatcaacattgatccattgaaattgtttgaaataaagaatacattgctatatttcaggaataatttaaatgttccctatcttggagtcttgatggatatactgctatcttgaattttaattctgggaatccttttatgccctggaattaaattctcaacaatcttttgacactttaagagctgagctgaaggttcatcaccttcattattttgacatctcctgtagctggctctcacttcaggatcctgagttgagaataaactagaagggaagattatataaagggatttccacctcttctgtctcaattaccattttaaaaaaataaaaagttttagaggaaaacacttagtagttcaccctttacccttgaccttccacggcagttttaaaataagcaaaggaaaagattcatgaattcaggccatagcctggggcctgagaacttttacttatgcaccttctcaggaagggtttcattgttaaatagaagggcaggacaggaaagttgggcctctttgttcttctcaatgtaacttctttatttggtttaaagtataaaatgtatacaacaacaaataaccacatttaaaatacacagtttgtttcccaacatcattttgctaagtcatagtggctccttaactgtaatttttttttttattagtccaagccttaggattatgttatctgtgatatatgttataatagaaaacttaagcctcttaaaacaaagtccttgggatgggacctaagattcacattatcttgattccgcataacagttgcttacattttagcaaatctccagtgtgtatgcaagcactcctcacttggcacaattctgatacacacaaactttggttaccccagttttgttatgtaacaccaccttcaacaacacagttcaaatttcagttatcatagtatattaactctgagtaacagcacaaagtacaaactccactgctagctcttcagtgtatagatcagttacctgagtaacagatgtgcaggctgagcaggctcactggtcagtcatgacactattttcagtctgttactaattggtcactgagcatctgctatccaattcacaaacaaagaaagcatgtagtgttgcttccttgtgttccagtaataagcccatgtgacattttacaaaaatggataattgaaaaagagaatgggtgcagtggctcacgcatgttgggaggccaaggcggatggatcacctgaggtcaggagttcgaaaccagcctggccaacatggtgaaaccccatctctactaaaaatacaaaattagctgagtgtggcgacagatgcctgtaatcccagctactggggaggctgaggcaggagaatcgcttgcacctaggaggcggaggttgcagtgagccgagatcgtgccattgcactccagcctgggtaacaagagcgaaactccgtctcaaaaaaaaaaaagaaaagaaaagaaaagaaaaagagaatgggctagcaaagaaatgaaaaatggtaacactggaagtgaaaatcaaaacagagtaatggatttatagaagaaatagctgagtgaagaagaaataggagtgttgacactgcgatcattcaagagatccagatatggagccagaagaacttagggcaggtctatcaacttaaatgaggaaaatagctgtgataaaacagatgaagatgtcttgaggaaatgatgcctgcaaaaaacttcacattaagggaagtcttattagagatatttcacaataatgaaagtacaaaagaaaaaatgttggggctggacatggtggcttactcctgtaatcccagcactttgagaggccaaggtgggtggatcacttgaggccaggaagtcgagacaagcctgatcaacatgatgaaaccccgtctctattaaaaatacaaaaattagccagacatgatggtgcacacctgtaattccagctactcaagtggctgaggcacgagaattgcttgaaccagggaggcggaggttgcagtaagctgagattgcaccactgcaatccagccaggtgacagggtaagactgtgtctcaaaaataaataaaagaaaaatatgttggaagctcatccacatttaagaaggaatatgacaattcactaatgcatagaaaagaagttcactccacattgtaaagtgtacagtgtaatattatacaatgaaaacaaggcaagtgctgtttaaactactctggatacattttttacaaagaaataaaacactttagtttttaatgtttctaatgttttacattttagtgtattaaatcaatattagttttcttcttttttaagctccctatacatttataactgacactaagggagtgtttaatgttttgattaaaagttgtaaagatcacagaacaattgtaattcttcccactgattattcagatcattttgcacaatttcagcttgcatggtcacttacagtgccgcactatgtgcaaagcaaggtcaggtctaaagttcgctaatgaaaaatcctcggccaggggcagtggctcacccctgtaatcctagcactttggaaggcgaggcaggcagatcgcttgagctcaggagttcaacaccagcctgggcaacatggtgagaccctgtctctacaaaaaaaaaaaaatagcaaggcgtggtgactcacacctgtagtcccagctacttgttgggggctgagttgggaaaatcacttgagctcaggaggtcgaggctgcagtgagccagaatcacgccactgcccttctgcctgggtgacagagtaagatcctgtctcaaaaaaagggaaaatcctcatctacatttcactgggttttttgtttgtttgtttgtttgtttatacacacttaaggaaattactgtctagaagatagataatataaaaaataaaaatgcaattcatgattcgggtttcttggtattcctaagaactgttgcacagtactttatgctctgaggcagacagctatagcatatatagtaatttttgtttctatcacataaacttgaatacacatatgagtaaaagacctttagttcttcatgacttactgaaagaccctgactttttccatgtaactgttccacaagtgttttatggaaaactggatacattaattcttcattcatccagcacgtacttgttgaatggccaatgtacaccaggtttgtagtagttactactgtgaatggaaagtaaaacagatgcaaaaggagaatacactaaaccaagtcttttattttttctctctctgatagggctaccataaagctttggtcaaactaaaactgaaaaaaaataccgtggatgacatttcagagagtctgcggcaaggaggaggcaagttaaactttgacgaacttcgacaagatctcaaagggtgagaatcatgcttcctgaggttctgaaaaattcctgcttctaaagataaattcctggtgataagagtatttctagcccaagggctcatacagatactttttttttttttttccagaggcaggtatctttctggaacatgttataagaggaaaacttgcccccatttggtgatttctcctttcctcctgcattttgatgtctctgtgttgagggtgaactgggtacaaggaatgatttttatctgtatcctctctctaatttcaggaagggccatactgatgcagagattgaggcaatattcacaaagtacgaccaagatggagaccaagaactgaccgaacatgaacatcagcagatgagagacgacttggagaaagagagggtgggtctggtttaggagaaccggatttgatttggtacctacaacaccacagatgtatcaaacactatagaagtagtgggttattgagtctcttgcccattccccaccacactctctctctctctcagtcggtttatgtgttagtaccctgtttattccagaaagaatatataacacaattatgtataaaaatgggtggttagcatgatataaaaacgtcaaaatgaaaagcaagcaaaacaaaagtaaaaataatggattattaatgaagcttaaaaatgcattcataaaaacacatatgcttattaagattgggctacaaattgggccctaagcttgctggtaatcagcttgaaaagagaagcctgattagctgcagagtccacaatgtccgtgagagtgaagaaaacaaaaaatgacttaccaagagatgtgaaattattctggttagttagtggctatttaaattgttaacttttttttcttttttttttttttttgagatggagtcttgctctgcctcccaggctggagtgcagtggcacaatcgcgactcactgcaacctccacctcccgggttcaagcgattctcttgcctcagcctcccaagtagctaggactacaggcacatgccatcatgcccggctaatttttgtatttgtagtagatatggggtttcaccatgttggtctcaaactcctgactgcaagcaatctgcccaccttggcctcccaaagtggtgggattacaggcagtagccaccgtgcctttcctaaattattaacatttataataaaattaacagccgccttccatttgaatactttttacaaaatagttaaaaataaacataagtgggcttttatagtcagaaaaaaaaattcaaagctttaccattaactttcaaaaataaatggttagacagcaacaacaaaaatctgtggtaactgaggtacagagaacacagatgaatgttattacaaaagccactttcctatgagaagtctaggacagtggtttctaaatgccactccacagacagtgctagtaggtgacagacttctccagtcacagtgaaatttaagcataaagaaaatgaggaaaatttttacaaggctctatttagacaaagttcttattctgacattacatctttcctactttggagctgttgaatgtattatcttttatgaaaagaaggcgatccaggttgagcatccctaacccaaatatgtgagtctgaaatgctccaaaacctaaaacttcttgagcacaaacatgatagtcaaaggtcatgcttaaaggaaatgctgtcattggagcagtttggattttgggttttcagattagggatgctgaaccagtaagtataatgcaaacattccaaaatatttttgaaaatcccaaatccaaaacacttctgatcccaagtatttcaaataagggatactcaacctgtaatatatttcttcatttctttatttattttattattattttaagatggctcatggcccactgcagcctcaaactcctaggctcaagtgatcttccgacctcaacctcccaggtagctcaggtagctgggactgcaggcatgcatcaccatgcctggctaattttttaaaaaattttttgtggaggcagagtctcaccttgctgcccaggccagtctcaaactcctggcttcaagcagtactcctgcctcagcctcccaaagtattaggattacaggtgtgaccactatgcctggcccatatttcttcatttagttttttctttgcctgctgtgtttttaatgttctttcttgttcaaacaaaaagttggctattccttgctgttagttaaatttgccaatctatgaaactgaaaaatgcaggagtcccagcctggtgttaaatacaaagaaatcccaggtaaatggcatgcacccagttcctgcttgcccaagtccttggtgaggcttctgtggggtctcagtgttctgctcctcactcagtgaccccttgttcttcaggaggacctggatttggatcacagttctttaccacgtcccatgagcagccgaagtttccctcgaagcctggatgactctgaggaggatgacgatgaagatagcggacatagctccagaaggaggggaagcatttctagtggcgtttcttacgaagagtttcaagtgtaagtataaaggaattggcagaatttgcgttgacaagagtccacatgagaccaggcagttccctcatctctctgaattcactcctttccattactaatcatccagcttttaaaaataacttatactggccagacgcagtggctcatgcctgtaatcccaccactttgggaggccaaagcaggcaggtcatgaggtcagaagttcgagacgagcctggccaacatagtgaaaccccatctctattaaaaatacaaaaaattagctgggcatggtggtgggcacctgtaatcctagctacttgggaggctgaggcaagagaattgcttgaacccgggaggcggaggttacagagagccgagatggctccactgcacaccagcctgggcgacagtgcaagactctgtctcaaaaaaaaaaaaacttgtcaattggtgttttgtttcttacataatatgtttactataaaaattagcaaataagagcaaaagaaaacattaacatttcacatatttctaccaatgaaaaatgtttattaatatatcagtgtttgtgtctctatttgcatgtgtttatcaatgtttctatatatttttatggcctaacatatggttcgtcctgaagaatgttccttgtgcatttgagaagaatgaatattctgctgttcaagtgttctgtagatgtttgttaggtctagtttgtttacagttttattcaggtctcccatttcctggttgatcttagatgtgcctagatgtggtattcacgtttgaaagtggggtattaaagtctccaagtattattagttggagttcatcccttcaattctgtaaggttttgctttgtgtattttggaactctgttgttgggtgcatacatatttataactagtatattttcctaatatattgaccctattttctctctcaacttaatgaggctaaagaaaaaaaagaattgaccctgttttcattacaagatgttatccactttatctctagtaaaattctttgttttaagtattttttgtttgatattactgtaaccactccagctttcttttggttgctgtttgcatgataaatctttttccatccttttactttaaacttatttatatctttcagtctgaagtatgtctctcctgtagacagcatataattggatcttatctttttatccagtttgacaatttctgtttttgattagattgcttaatccattcatttaatgttatcattgatgtagttgatttctgtctgctattttattttttgttttctagcttacttttttttgttcctctttcactgctttcttgtacattaagtgaatattttcaagtataacatttaaatttttttaatgatttttcattttttttagtcaggagttgctctaagacttagtttatacaattaagttatgaaaaattacttcagatatatattaactgaatccagtgagatatagaaatcatttctatttagcttttttcctcttccctctttttgtgctatatattcatctatctatatgtatatatagtcatctacatatgttgcaaatcacattgttagaacaatgttacatttttataacacactgtgtaatatatagtatataattttatatctcttaatgaagctgagagaagaggaagtatatatttataaatgtgtatattaacctacttttttaccatttctaattctcttcttttgctcctctggattcaagttatcatctgtcgtcattctctttctccgatacagctttgctactgcctacctcctattattgtcaaatatattacatttctattatagaccttcagatccaattatgtacatatttttacacaactgctttttaaatcagttaaggaacaaaaggagaaatgtacatatatactatattttatacctacacagttatctttaccagtgttctttgccttttcatgtggattctgattactatctggagtcacttgctttcagcataaagaatttcctttagtattttttgtaaagcaggtttgctagcaatgaattctttcattttttgtttatctgagaatgtttttctttctccttcatttcctctggcttgtattgtttctgatgagaagacaggtgctaattttactgtggtccccttgtacatgatgactcaattttctctcaccactttcaagatttttttgcttttgtctttcattatttttactgtgatatgtctgggtataaatctctgagttcatcctacttagaaagtgttttttctgcttctttcactttctcttctcctttgggacccgcattatgcatatgcttaggggtatcacatatttctcttaggctctgttcgtcgtcattttttttccctctctgttcctcagagtgcatagtctgtattgatgtatcttcaagttcactgacttgttcttctgtcagcttgcttaaatctctgttgagctcctctagttatttatttatttatttatttattttatatatatatatatatatatatatatagagagagagagagagagagagagagaaagagagagagagagagacagggagacagggtctcactccatcacccaggctggagtacagtggtgtaatcatggctctctacagcctgaacacctgggctcaaatgatcctcctgcctcaacctcccaagtagctaggactatgggcacatgctgccatgcctggctaatttttaaaaaaaaatttgtagagatggcatcttgttatgttgcccaggttggtctcaaactcccggcctcaagtgatccttccgcctcggcctcccaaagtgctggggtcacaggtatgagccaccgcacttagcctgaattttttatttattatacttttcaactccagaatttctatttggttctttttagcaatctctgtctctttattctgtatttgatgatatcctgtatttgatgagacaatgtcatcataacttccttttttttttaagagatagggtctctctctgtcacccaggctagagtgcagtggcatgatcctagctcactgcagcctcgaactcctgaactcaagcaatcctcccacctcagcctcctaagtacctgacactacaggcatgagccactgtacccagctaatttttattttttgtagagatggggtctaagttgcccaggctggtctcaaactcctgggctcaagtgatcctccctgctcaggctcccaaagtgctgggattacaggcatgaaccactggacccagcctcctttatttctttaaccatggtggggttttttattatttgtttgtttgttttaacctgtttgaatatatttataataggtaccttcaggtctttgtctgctaagtctgacatctgggccctctcaaagacagtttcagttgtctttttttttttcttcttgtgtatgggaacattttcctgtttttttgttttgttttttgttgttttgttttgttgttgttgttgttgttaaagattggacatgttagataatatattgtaggaactctggctagtgattccctttcctccccaggacttgtttttatttctctttgcttgtttactttttcatacagtctgtttccccacagtgtctgcctctgactttattccttagagggcgcagctgtggccatgtacgtagtcactctgggaagacagtggttttagcagcgtgctcattaactttctctgacctctttgttatacctcctgcctctgtggatattagacccagttattacatttcattgtttactgattggtctattgttttccagaatgccttgggatgtaatttgctccacagtctgatccacttaaattcaggcctctttgcagggctagttttagaggccagtctttttttttttttttttttttttttgagacagagtctcactctgttgcccaggccggaatgtagtggtgcagtctcagctcactgcagcctcaatctctcaggctcaagcaatcttcccacctcagcttctcaagtagctaggactacaggtgcacgccaccatgcctggttaatttttgcattttttatagagatggggttttaccatgttgccccggctggtctcaaactcctgagttcaagcaatccacccacctcagcctcacaaaaggccgggattacagatatgcaccaccacgcctggcccctgaggccagtctttaactgtcttcttagctgtctctttccctggttctctctggtgaactagctggtaatttgtttatctcataaggctaccagattccttgtaaatgcttatccccacaatctccattgttttcaagagcatcgttagtctttaatttcctcacgcttcattccaaataaagtccattcacttgagaagagttctatattcctatgacctgtgtctccccatgagcaaaactgctactgctttacagagccagggacagtggcccacctctctgtggcatcctgctttatgaacaagtcactgggctcagatggcagtctctgattttctcaccttgcttcttctggcatggaaactccaccctacaagtgggaactgagtggaagaagagagccccattccccttagccactcttaacaggattagaacctctgcaacatgcatttaagaatgggaacaggctgggcacagtggctcacgcctgtaatcctagcactttgggaggtcaaggcaggaggattgcttgagccaggagttcaggaccagcctgtgcaacatggtgagaccctcatctctacaagaaatagaaaaattaactggtgggttgtgtatacctgtaatcccagctactcgggagcctgaagtgggaggattgcttgaacctggaggcagaggttgcagtgagtcaagattacaccattgcacgccagcctgggcaacagagtgagactctgtctcaaaaaaaaaaaaaaaaaaagagtaggaacattgaggctgggcatggtggctcatgcctgtaatcctagcactatgggaggccaaggcaggagtatcacttgacgctaggagttcaagaccaacctgggtaacatagcgagactttgtctctattaaaaaaaaaaaaaaaaaaggtgtaaaaaacttttgtaacaagagtgggaaagccgggcacagtggctcacacctgtaatcccagcactttggaaggccaaggcaggcaggcggatcacctgaggtcaggagtttgagaccagcctggccaacgtggtgaaaccccatctctactaaaaaatagaaaaattatctgggcatggtggtgcacacctgtagtcccagctactcgggaggctgaggcaggagaatcacttgaacctacgaggcagaagttgcagtaagccaagatcacgccactgcactccagcctgggcgacagagcaagactctgtctcaaaaaaaaaaaaagagtggaaatgttaggatgagaaatgctggcagcctgcccctccctgggagatactgtagccctagactggaagttgggggaggagggagccctgtgctcttagctgcacccatgtgaagttgtgcttctatcacatgagctggggacaggagagaaggctcagattatggcttcagtgccacagaatctcttcatactaaaatttagtagattttcttgaataaatgctttttcatttgctgtacacccttaaaagtttctagaaatttttaatatttgagttttaaaaaataattttcaacagttacagttatttcactaaagagagagtctacagaaccctcttgccaccattgcagaggttgtctttggcttacgagtttttaaagtatttgtatacattttttaagttcaaaataatagaattgtaagtgaacatgctgttttcatactgtttttcaagctttatttaatatattgtaaatctaattctattttattaaatagtctgccacagtataatgtctgatgtctccttagaattttattgtatggatgaacaatgattatttaatttcctaccaattgttgggtgttttttgtttgtttgtttgtttttgagactgggtctcactctgtcacccaggctggagtgcaggagtgcggtggaatgatcacggctcactgcagcctcaacatcccaaggctcaggtgatccttccacctcagcctcgcaagtagctgggagtacaggcacatgccaccatgcccacctattttttagagatgaagttttgccatcctgcccaggctggctcgaactcctggcctcaagcgatctgcacacttccgcctcccaaaatgccaggattacaggcgtgagccatcatgccctacccccccatcaattgtttgatgtagccatttttcaatgatccgcgattaagaagcagcactcttttatagccaaaaattacacatatataaaattttcctttagaaaatgttctaaaaatggaatgtctaactaaagggttaggcatacattcttaagacttctgatacgcgctgacttgcaggaaagttgtttcagttaacactcctaccagcggcatccgagagttaatctgtaaagcttgagacaacttagaaagtgtttcaaatgattgtgttgcttaagaaaaaaatcttagcacttccttttgaaaagccagtggggctgaaaagacaatgacaagcactttgtccctctgtactgtgttttccttgcagcctggtgagacgagtggaccggatggagcattccatcggcagcatagtgtccaagattgacgccgtgatcgtgaagctagagattatggagcgagccaaactgaagaggagggaggtgctgggaaggctgttggatggggtggccgaggtcagtagtcatgagctgaaaacaccgctgctgagcatggtgttattaatgaaaatatatgttgctgacagttgtatttgaagtattgaagaagagtaaaaaaaatttacgtttatagaaattcacaatgatgtttccatttactctcattttcagatttttttctctgaaacagaaacactctttctataaaatctcttgctataaaacatcaatgtagtcatattgtctaacccttaggctgagatgtttatctttctccataactacagataaaattataatctggaggtgttactttcttaatactccatatgctaatggtcctgccttcactgcagggtagaattaagtgaaaaattactccagcaactctgagatttgctattatatgctgtaaatctccagccttaccaaactacagattatttggtccctggacttcctaaggcatttccttctactgcccccaacaccagtttctttttccctttttaggatgaaaggctgggtcgtgacagtgaaatccatagggaacagatggaacggctagtacgtgaagagttggaacgctgggaatccgatgatgcagcttcccagatcagtcatggtttaggcacgccagtgggactaaatggtcaacctcgccccagaagctcccgcccatcttcctcccaatctacagaaggcatggaaggtgcaggtggaaatgggagttctaatgtccacgtatgatatgtgtgtttcagtatgtgtgtttctaataagtgaggaagtggctgtcctgaattgctgtaacaagcacactatttatatgccctgaccaccataggatgctagtctttgtgaccgattgctaatcttctgcactttaatttattttatataaactttacccatggtt

SEQ ID NO: 244 - DNA, 5089 bp, Homo sapiens
  source 1..5089: mol_type = genomic DNA
aggcggcggcgggcgccgggaagaaaggaacatggctcctgaggcgcacagcgccgagcgcggcgccgcgcacccgcgcgccggacgccagtgaccgcgatggtgaactccagtcgcgtgcagcctcagcagcccggggacgccaagcggccgcccgcgccccgcgcgccggacccgggccggctgatggctggctgcgcggccgtgggcgccagcctcgccgccccgggcggcctctgcgagcagcggggcctggagatcgagatgcagcgcatccggcaggcggccgcgcgggaccccccggccggagccgcggcctccccttctcctccgctctcgtcgtgctcccggcaggcgtggagccgcgataaccccggcttcgaggccgaggaggaggaggaggaggtggaaggggaagaaggcggaatggtggtggagatggacgtagagtggcgcccgggcagccggaggtcggccgcctcctcggccgtgagctccgtgggcgcgcggagccgggggcttgggggctaccacggcgcgggccacccgagcgggaggcggcgccggcgagaggaccagggcccgccgtgccccagcccagtcggcggcggggacccgctgcatcgccacctccccctggaagggcagccgccccgagtggcctgggcggagaggctggttcgcgggctgcgaggtctctggggaacaagactcatggaggaaagcagcactaaccgagagaaataccttaaaagtgttttacgggaactggtcacatacctcctttttctcatagtcttgtgcatcttgacctacggcatgatgagctccaatgtgtactactacacccggatgatgtcacagctcttcctagacacccccgtgtccaaaacggagaaaactaactttaaaactctgtcttccatggaagacttctggaagttcacagaaggctccttattggatgggctgtactggaagatgcagcccagcaaccagactgaagctgacaaccgaagtttcatcttctatgagaacctgctgttaggggttccacgaatacggcaactccgagtcagaaatggatcctgctctatcccccaggacttgagagatgaaattaaagagtgctatgatgtctactctgtcagtagtgaagatagggctccctttgggccccgaaatggaaccgcttggatctacacaagtgaaaaagacttgaatggtagtagccactggggaatcattgcaacttatagtggagctggctattatctggatttgtcaagaacaagagaggaaacagctgcacaagttgctagcctcaagaaaaatgtctggctggaccgaggaaccagggcaacttttattgacttctcagtgtacaacgccaacattaacctgttctgtgtggtcaggttattggttgaattcccagcaacaggtggtgtgattccatcttggcaatttcagcctttaaagctgatccgatatgtcacaacttttgatttcttcctggcagcctgtgagattatcttttgtttctttatcttttactatgtggtggaagagatattggaaattcgcattcacaaactacactatttcaggagtttctggaattgtctggatgttgtgatcgttgtgctgtcagtggtagctataggaattaacatatacagaacatcaaatgtggaggtgctactacagtttctggaagatcaaaatactttccccaactttgagcatctggcatattggcagatacagttcaacaatatagctgctgtcacagtattttttgtctggattaagctcttcaaattcatcaattttaacaggaccatgagccagctctcgacaaccatgtctcgatgtgccaaagacctgtttggctttgctattatgttcttcattattttcctagcgtatgctcagttggcataccttgtctttggcactcaggtcgatgacttcagtactttccaagagtgtatcttcactcaattccgtatcattttgggcgatatcaactttgcagagattgaggaagctaatcgagttttgggaccaatttatttcactacatttgtgttctttatgttcttcattcttttgaatatgtttttggctatcatcaatgatacttactctgaagtgaaatctgacttggcacagcagaaagctgaaatggaactctcagatcttatcagaaagggctaccataaagctttggtcaaactaaaactgaaaaaaaataccgtggatgacatttcagagagtctgcggcaaggaggaggcaagttaaactttgacgaacttcgacaagatctcaaagggaagggccatactgatgcagagattgaggcaatattcacaaagtacgaccaagatggagaccaagaactgaccgaacatgaacatcagcagatgagagacgacttggagaaagagagggaggacctggatttggatcacagttctttaccacgtcccatgagcagccgaagtttccctcgaagcctggatgactctgaggaggatgacgatgaagatagcggacatagctccagaaggaggggaagcatttctagtggcgtttcttacgaagagtttcaagtcctggtgagacgagtggaccggatggagcattccatcggcagcatagtgtccaagattgacgccgtgatcgtgaagctagagattatggagcgagccaaactgaagaggagggaggtgctgggaaggctgttggatggggtggccgaggatgaaaggctgggtcgtgacagtgaaatccatagggaacagatggaacggctagtacgtgaagagttggaacgctgggaatccgatgatgcagcttcccagatcagtcatggtttaggcacgccagtgggactaaatggtcaacctcgccccagaagctcccgcccatcttcctcccaatctacagaaggcatggaaggtgcaggtggaaatgggagttctaatgtccacgtatgatatgtgtgtttcagtatgtgtgtttctaataagtgaggaagtggctgtcctgaattgctgtaacaagcacactatttatatgccctgaccaccataggatgctagtctttgtgaccgattgctaatcttctgcactttaatttattttatataaactttacccatggttcaaagatttttttttctttttctcatataagaaatctaggtgtaaatattgagtacagaaaaaaaatcttcatgatgtgtattgagcggtacgcccagttgccaccatgactgagtcttctcagttgacaatgaagtagccttttaaagctagaaaactgtcaaagggcttctgagtttcatttccagtcacaaaaatcagtattgttatttttttccaagagtgtgaaggaaaatggggcattcctttccactctggcatagttcatgagcttaatacatagctttcttttaagaaaggagccttttttttcaactagcttcctggggtaaacttttctaaaagataaaatggaaaggaactccaaactatgatagaatctgtgtgaatggttaagatgaatgttaaatactatgcttttttgtaagttgatcgtatctgatgtctgtgggactaactgtatcacttaatttttaccttattttggctctaatttgaataagctgagtaaaaccaccaaagatcagttataggataaaatggcatctctaaccataacacaggagaattggaaggagccctaagttgtcactcagtttaatttcttttaatggttagtttagcctaaagatttatctgcatattctttttcccatgtggctctactcatttgcaactgaatttaatgttataactcatctagtgagaccaacttactaaatttttagtatgcactgaaagtttttatccaacaattatgttcattttaagcaaaattttaagaaagttttgaaattcataaagcatttggttttaaactattttaagaatatagtactcggtcaggtatgacggctcacgcctgtaatcccagcactttgggaggccgaaacaggcgaatcacttgagcccaggagttcaagaccaacatgggcaatgtggcgaaactccatctctacaaaaaatgcaaaaataaaaaatatagtactcaagtattcttgatcctgtgtttcaaaactagaatttgtaatgcaaatggagctcagtctaataaaaaagaggttttggtattaaaagttcatacattagacagtatcagccaaaatttgagttagcaacactgttttctttacgagagggtctcacccaaatttatggggagaaatctatttctcaaaaaaaaaaatcttcttttacagaaatgttgagtaaggtgacattttgagcgctaataagcaaaagagcatgcagtgctgttgaataaccctcacttggagaaccaagagaatcctgtcgtttaatgctatattttaatttcacaagttgttcatttaactggtagaatgtcagtccaatctccaatgagaacatgagcaaatagacctttccaggttgaaagtgaaacatactgggtttctgtaagtttttcctcatggcttcatctctatctttactttctcttgaatatgctacacaaagttctttattactacatactaaagtttgcattccagggatattgactgtacatatttatgtatatgtaccatgttgttacatgtaaacaaacttcaatttgaagtgcagctattatgtggtatccatgtgtatcgaccatgtgccatatatcaattatggtcactagaaagtctctttatgatactttttattgtactgtttttcatttcacttgcaaaattttgcagaattcctcctttctacccataaattacatatatttttcttctttagtcatggagaactccccccctcatctcttccctattatctttccctgtgtactggtattattaaaaagacattacatacgcaagtctttctcgacaatcaagaatgttattaatgtgtaatactgagcactttacttcttaataaaaacttgatatagtagca

SEQ ID NO: 245 - DNA, 1222 bp, Homo sapiens
  source 1..1222: mol_type = genomic DNA
atgtcttctctctcttacagctcttcaaattcatcaattttaacaggaccatgagccagctctcgacaaccatgtctcgatgtgccaaagacctgtttggctttgctattatgttcttcattattttcctagcgtatgctcagttggcataccttgtctttggcactcaggtcgatgacttcagtactttccaagagtgtatcttcactcaattccgtatcattttgggcgatatcaactttgcagagattgaggaagctaatcgagttttgggaccaatttatttcactacatttgtgttctttatgttcttcattcttttgaatatgtttttggctatcatcaatgatacttactctgaagtgaaatctgacttggcacagcagaaagctgaaatggaactctcagatcttatcagaaagggctaccataaagctttggtcaaactaaaactgaaaaaaaataccgtggatgacatttcagagagtctgcggcaaggaggaggcaagttaaactttgacgaacttcgacaagatctcaaagggaagggccatactgatgcagagattgaggcaatattcacaaagtacgaccaagatggagaccaagaactgaccgaacatgaacatcagcagatgagagacgacttggagaaagagagggaggacctggatttggatcacagttctttaccacgtcccatgagcagccgaagtttccctcgaagcctggatgactctgaggaggatgacgatgaagatagcggacatagctccagaaggaggggaagcatttctagtggcgtttcttacgaagagtttcaagtcctggtgagacgagtggaccggatggagcattccatcggcagcatagtgtccaagattgacgccgtgatcgtgaagctagagattatggagcgagccaaactgaagaggagggaggtgctgggaaggctgttggatggggtggccgaggatgaaaggctgggtcgtgacagtgaaatccatagggaacagatggaacggctagtacgtgaagagttggaacgctgggaatccgatgatgcagcttcccagatcagtcatggtttaggcacgccagtgggactaaatggtcaacctcgccccagaagctcccgcccatcttcctcccaatctacagaaggcatggaaggtgcaggtggaaatgggagttctaatgtccacgtatgatatgtgtgttt

SEQ ID NO: 246 - DNA, 693 bp, Homo sapiens
  source 1..693: mol_type = genomic DNA
gaatgataggggaaaggaaggcaagggtgagagaagaccttgtgtgaatttgtccaaaatgtttatccacaggaacaatccctttgtgaaggctgctggtatgtgaatgtgtgccggttcccttggggcgttcatttggatctttctgtgttccagtgacctacggcatgatgagctccaatgtgtactactacacccggatgatgtcacagctcttcctagacacccccgtgtccaaaacggagaaaactaactttaaaactctgtcttccatggaagacttctggaagttcacagaaggctccttattggatgggctgtactggaagatgcagcccagcaaccagactgaagctgacaaccgaagtttcatcttctatgagaacctgctgttaggggttccacgaatacggcaactccgagtcagaaatggatcctgctctatcccccaggacttgagagatgaaattaaagagtgctatgatgtctactctgtcagtagtgaagatagggctccctttgggccccgaaatggaaccgcttggatctacacaagtgaaaaagacttgaatggtagtagccactggggaatcattgcaacttatagtggagctggctattatctggatttgtcaagaacaagagaggaaacagctgcacaagttgctagcctcaagaaaaatgtctggctgg

SEQ ID NO: 247 - DNA, 550 bp, Homo sapiens
  source 1..550: mol_type = genomic DNA
ccagtcggcggcggggacccgctgcatcgccacctccccctggaagggcagccgccccgagtggcctgggcggagaggctggttcgcgggctgcgaggtctctggggaacaagactcatggaggaaagcagcactaaccgagagaaataccttaaaagtgttttacgggaactggtcacatacctcctttttctcatagtcttgtgcatctgaacaatccctttgtgaaggctgctggtatgtgaatgtgtgccggttcccttggggcgttcatttggatctttctgtgttccagtgacctacggcatgatgagctccaatgtgtactactacacccggatgatgtcacagctcttcctagacacccccgtgtccaaaacggagaaaactaactttaaaactctgtcttccatggaagacttctggaagttcacagaaggctccttattggatgggctgtactggaagatgcagcccagcaaccagactgaagctgacaaccgaagtttcatcttctatgagaacctgctgttaggggttccacgaatac

SEQ ID NO: 248 - DNA, 1720 bp, Homo sapiens
  source 1..1720: mol_type = genomic DNA
aatggtagtagccactggggaatcattgcaacttatagtggagctggctattatctggatttgtcaagaacaagagaggaaacagctgcacaagttgctagcctcaagaaaaatgtctggctggaccgaggaaccagggcaacttttattgacttctcagtgtacaacgccaacattaacctgttctgtgtggtcagctgtcagtggtagctataggaattaacatatacagaacatcaaatgtggaggtgctactacagtttctggaagatcaaaatactttccccaactttgagcatctggcatattggcagatacagttcaacaatatagctgctgtcacagtattttttgtctggattaagctcttcaaattcatcaattttaacaggaccatgagccagctctcgacaaccatgtctcgatgtgccaaagacctgtttggctttgctattatgttcttcattattttcctagcgtatgctcagttggcataccttgtctttggcactcaggtcgatgacttcagtactttccaagagtgtatcttcactcaattccgtatcattttgggcgatatcaactttgcagagattgaggaagctaatcgagttttgggaccaatttatttcactacatttgtgttctttatgttcttcattcttttgaatatgtttttggctatcatcaatgatacttactctgaagtgaaatctgacttggcacagcagaaagctgaaatggaactctcagatcttatcagaaagggctaccataaagctttggtcaaactaaaactgaaaaaaaataccgtggatgacatttcagagagtctgcggcaaggaggaggcaagttaaactttgacgaacttcgacaagatctcaaagggaagggccatactgatgcagagattgaggcaatattcacaaagtacgaccaagatggagaccaagaactgaccgaacatgaacatcagcagatgagagacgacttggagaaagagagggaggacctggatttggatcacagttctttaccacgtcccatgagcagccgaagtttccctcgaagcctggatgactctgaggaggatgacgatgaagatagcggacatagctccagaaggaggggaagcatttctagtggcgtttcttacgaagagtttcaagtcctggtgagacgagtggaccggatggagcattccatcggcagcatagtgtccaagattgacgccgtgatcgtgaagctagagattatggagcgagccaaactgaagaggagggaggtgctgggaaggctgttggatggggtggccgaggatgaaaggctgggtcgtgacagtgaaatccatagggaacagatggaacggctagtacgtgaagagttggaacgctgggaatccgatgatgcagcttcccagatcagtcatggtttaggcacgccagtgggactaaatggtcaacctcgccccagaagctcccgcccatcttcctcccaatctacagaaggcatggaaggtgcaggtggaaatgggagttctaatgtccacgtatgatatgtgtgtttcagtatgtgtgtttctaataagtgaggaagtggctgtcctgaattgctgtaacaagcacactatttatatgccctgaccaccataggatgctagtctttgtgaccgattgctaatcttctgcactttaatttattttatataaactttaccca

SEQ ID NO: 249 - DNA, 1362 bp, Homo sapiens
  source 1..1362: mol_type = genomic DNA
atactttccccaactttgagcatctggcatattggcagatacagttcaacaatatagctgctgtcacagtattttttgtctggattaagctcttcaaattcatcaattttaacaggaccatgagccagctctcgacaaccatgtctcgatgtgccaaagacctgtttggctttgctattatgttcttcattattttcctagcgtatgctcagttggcataccttgtctttggcactcaggtcgatgacttcagtactttccaagagtgtataatatgtttttggctatcatcaatgatacttactctgaagtgaaatctgacttggcacagcagaaagctgaaatggaactctcagatcttatcagaaagggctaccataaagctttggtcaaactaaaactgaaaaaaaataccgtggatgacatttcagagagtctgcggcaaggaggaggcaagttaaactttgacgaacttcgacaagatctcaaagggaagggccatactgatgcagagattgaggcaatattcacaaagtacgaccaagatggagaccaagaactgaccgaacatgaacatcagcagatgagagacgacttggagaaagagagggaggacctggatttggatcacagttctttaccacgtcccatgagcagccgaagtttccctcgaagcctggatgactctgaggaggatgacgatgaagatagcggacatagctccagaaggaggggaagcatttctagtggcgtttcttacgaagagtttcaagtcctggtgagacgagtggaccggatggagcattccatcggcagcatagtgtccaagattgacgccgtgatcgtgaagctagagattatggagcgagccaaactgaagaggagggaggtgctgggaaggctgttggatggggtggccgaggatgaaaggctgggtcgtgacagtgaaatccatagggaacagatggaacggctagtacgtgaagagttggaacgctgggaatccgatgatgcagcttcccagatcagtcatggtttaggcacgccagtgggactaaatggtcaacctcgccccagaagctcccgcccatcttcctcccaatctacagaaggcatggaaggtgcaggtggaaatgggagttctaatgtccacgtatgatatgtgtgtttcagtatgtgtgtttctaataagtgaggaagtggctgtcctgaattgctgtaacaagcacactatttatatgccctgaccaccataggatgctagtctttgtgaccgattgctaatcttctgcactttaatttattttatataaactttacccatggttcaaagatttttttttctttttctcatataagaaa

SEQ ID NO: 250 - DNA, 1554 bp, Homo sapiens
  source 1..1554: mol_type = genomic DNA
acagttcaacaatatagctgctgtcacagtattttttgtctggattaagctcttcaaattcatcaattttaacaggaccatgagccagctctcgacaaccatgtctcgatgtgccaaagacctgtttggctttgctattatgttcttcattattttcctagcgtatgctcagttggcataccttgtctttggcactcaggtcgatgacttcagtactttccaagagtgtataatatgtttttggctatcatcaatgatacttactctgaagtgaaatctgacttggcacagcagaaagctgaaatggaactctcagatcttatcagaaagggctaccataaagctttggtcaaactaaaactgaaaaaaaataccgtggatgacatttcagagagtctgcggcaaggaggaggcaagttaaactttgacgaacttcgacaagatctcaaagggtgagaatcatgcttcctgaggttctgaaaaattcctgcttctaaagataaattcctggtgataagagtatttctagcccaagggctcatacagatactttttttttttttttccagaggcaggtatctttctggaacatgttataagaggaaaacttgcccccatttggtgatttctcctttcctcctgcattttgatgtctctgtgttgagggtgaactgggtacaaggaatgatttttatctgtatcctctctctaatttcaggaagggccatactgatgcagagattgaggcaatattcacaaagtacgaccaagatggagaccaagaactgaccgaacatgaacatcagcagatgagagacgacttggagaaagagagggaggacctggatttggatcacagttctttaccacgtcccatgagcagccgaagtttccctcgaagcctggatgactctgaggaggatgacgatgaagatagcggacatagctccagaaggaggggaagcatttctagtggcgtttcttacgaagagtttcaagtcctggtgagacgagtggaccggatggagcattccatcggcagcatagtgtccaagattgacgccgtgatcgtgaagctagagattatggagcgagccaaactgaagaggagggaggtgctgggaaggctgttggatggggtggccgaggatgaaaggctgggtcgtgacagtgaaatccatagggaacagatggaacggctagtacgtgaagagttggaacgctgggaatccgatgatgcagcttcccagatcagtcatggtttaggcacgccagtgggactaaatggtcaacctcgccccagaagctcccgcccatcttcctcccaatctacagaaggcatggaaggtgcaggtggaaatgggagttctaatgtccacgtatgatatgtgtgtttcagtatgtgtgtttctaataagtgaggaagtggctgtcctgaattgctgtaacaagcacactatttatatgccctgaccaccataggatgctagtctttgtgaccgattgctaatcttctgcactttaatttattttatataaactttacccatggtt

SEQ ID NO: 251 - DNA, 99 bp, Homo sapiens
  source 1..99: mol_type = genomic DNA
aggcggcggcgggcgccgggaagaaaggaacatggctcctgaggcgcacagcgccgagcgcggcgccgcgcacccgcgcgccggacgccagtgaccgcg

SEQ ID NO: 252 - RNA, 341 nt, synthetic construct
  misc_feature 1..341: note = Description of Artificial Sequence: Synthetic polynucleotide
  source 1..341: mol_type = other RNA
cccacatcgcctgccactacttaagtccgattcacttcggctttagctccaagcctttaatctcgcgaagctctttttttttttttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgc

SEQ ID NO: 253 - RNA, 20 nt, synthetic construct
  misc_feature 1..20: note = Description of Artificial Sequence: Synthetic oligonucleotide
  source 1..20: mol_type = other RNA
aagtgttacagctcttttag

SEQ ID NO: 254 - RNA, 42 nt, synthetic construct
  misc_feature 1..42: note = Description of Artificial Sequence: Synthetic oligonucleotide
  source 1..42: mol_type = other RNA
aatttgtctagcaggttttctgacttcggtcggaaaacccct

SEQ ID NO: 255 - RNA, 42 nt, synthetic construct
  misc_feature 1..42: note = Description of Artificial Sequence: Synthetic oligonucleotide
  source 1..42: mol_type = other RNA
aatttttggagcaggttttctgacttcggtcggaaaacccct

SEQ ID NO: 256 - RNA, 168 nt, synthetic construct
  misc_feature 1..168: note = Description of Artificial Sequence: Synthetic polynucleotide
  source 1..168: mol_type = other RNA
cccaatttcactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctgg

SEQ ID NO: 257 - RNA, 571 nt, synthetic construct
  misc_feature 1..571: note = Description of Artificial Sequence: Synthetic polynucleotide
  source 1..571: mol_type = other RNA
cccacatcgcctgccactacttaagtccgattcacttcggctttagctccaagcctttaatctcgcgaagctctttttttttttttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaagtgttacagctcttttagaatttgtctagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctgg

SEQ ID NO: 258 - RNA, 571 nt, synthetic construct
  misc_feature 1..571: note = Description of Artificial Sequence: Synthetic polynucleotide
  source 1..571: mol_type = other RNA
cccacatcgcctgccactacttaagtccgattcacttcggctttagctccaagcctttaatctcgcgaagctctttttttttttttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaagtgttacagctcttttagaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctgg

SEQ ID NO: 259 - RNA, 210 nt, synthetic construct
  misc_feature 1..210: note = Description of Artificial Sequence: Synthetic polynucleotide
  source 1..210: mol_type = other RNA
aatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctgg

SEQ ID NO: 260 - RNA, 569 nt, synthetic construct
  misc_feature 1..569: note = Description of Artificial Sequence: Synthetic polynucleotide
  source 1..569: mol_type = other RNA
cccacatcgcctgccactacttaagtccgattcacttcggctttagctccaagcctttaatctcgcgaagctctttttttttttttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcgtactttcaaagttatttaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctgg

SEQ ID NO: 261 - RNA, 569 nt, synthetic construct
  misc_feature 1..569: note = Description of Artificial Sequence: Synthetic polynucleotide
  source 1..569: mol_type = other RNA
cccacatcgcctgccactacttaagtccgattcacttcggctttagctccaagcctttaatctcgcgaagctctttttttttttttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcataaatcgaaccatcgtaaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctgg

SEQ ID NO: 262 - RNA, 569 nt, synthetic construct
  misc_feature 1..569: note = Description of Artificial Sequence: Synthetic polynucleotide
  source 1..569: mol_type = other RNA
cccacatcgcctgccactacttaagtccgattcacttcggctttagctccaagcctttaatctcgcgaagctctttttttttttttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgctttcttcccggcgcccgcaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctgg

SEQ ID NO: 263 - DNA, 121 bp, Homo sapiens
  source 1..121: mol_type = genomic DNA
ttacgatggttcgatttatgatttttcaacttcatggtgatgtgaaagtgatacacattctatagaaaccacactttcaattttgaattttggtctttttccaggctaccatactctaaag

SEQ ID NO: 264 - DNA, 94 bp, Homo sapiens
  source 1..94: mol_type = genomic DNA
tggtctttttccaggctaccatactctaaagatagagccacagatcccagtcagccatgtgattatgagggtaagcgaccaatactctacagtg

SEQ ID NO: 265 - DNA, 116 bp, Homo sapiens
  source 1..116: mol_type = genomic DNA
gattatgagggtaagcgaccaatactctacagtgtattgtattgccagatggttttgcccaactagcctaatgtaagtattctaaacatgtttaaggtaggccaggctaagctgtg

SEQ ID NO: 266 - DNA, 108 bp, Homo sapiens
  source 1..108: mol_type = genomic DNA
ggaaggtatttgcaaataactttgaaagtacctctctatcacagaaaattgttcatttggcttcatcatttcaatgcatgagtatcgacaggacctgctttgcattta

SEQ ID NO: 267 - DNA, 97 bp, Homo sapiens
  source 1..97: mol_type = genomic DNA
acctgctttgcatttaacactgtgtgagacgtaagttatggtgagttgttagaagttactgttcctactctcaaagggggtaaactaacattgagaa

SEQ ID NO: 268 - DNA, 102 bp, Homo sapiens
  source 1..102: mol_type = genomic DNA
aaactaacattgagaactttgcctgtgccttgcactgtgctgagtgtttcatatcttaccttatttaatttctatagtctaactctataaggtaagtactaa

SEQ ID NO: 269 - DNA, 150 bp, Homo sapiens
  source 1..150: mol_type = genomic DNA
aaggtatttgcaaataactttgaaagtacctctctatcacagaaaattgttcatttggcttcatcatttcaatgcatgagtatcgacaggacctgctttgcatttaacactgtgtgagacgtaagttatggtgagttgttagaagttact

SEQ ID NO: 270 - DNA, 146 bp, Homo sapiens
  source 1..146: mol_type = genomic DNA
tatggtgagttgttagaagttactgttcctactctcaaagggggtaaactaacattgagaactttgcctgtgccttgcactgtgctgagtgtttcatatcttaccttatttaatttctatagtctaactctataaggtaagtacta

SEQ ID NO: 271 - DNA, 98 bp, Homo sapiens
  source 1..98: mol_type = genomic DNA
ggcggcgggcgccgggaagaaaggaacatggctcctgaggcgcacagcgccgagcgcggcgccgcgcacccgcgcgccggacgccagtgaccgcgatg